(19) United States
(12) Patent Application Publication
(10) Pub. No.: US 2026/0062396 A1
(43) Pub. Date: Mar. 5, 2026

(54) ORGANIC COMPOUNDS AND ORGANIC LIGHT EMITTING DIODE COMPRISING THE SAME

(71) Applicant: Material Science Co., Ltd., Hwaseong-si (KR)

(72) Inventors: Tae Wan LEE, Hwaseong-si (KR); Ha Ni JEON, Hwaseong-si (KR); Da Hee PARK, Hwaseong-si (KR); Sang Mee KIM, Hwaseong-si (KR); Seung Uk CHO, Hwaseong-si (KR)

(21) Appl. No.: 19/311,639

(22) Filed: Aug. 27, 2025

(30) Foreign Application Priority Data
Sep. 3, 2024 (KR) 10-2024-0119102
Aug. 11, 2025 (KR) 10-2025-0110838

(51) Int. Cl. C07D 307/81; H10K 50/15; H10K 85/60

(52) U.S. Cl. CPC C07D 307/81; H10K 50/15; H10K 85/626; H10K 85/633; H10K 85/636; H10K 85/6572; H10K 85/6574; H10K 85/6576

(57) ABSTRACT

The organic compound represented by Chemical Formula 1 according to the present disclosure may exhibit excellent hole transport properties. Further, the hole transport layer or hole transport auxiliary layer of the organic light emitting diode according to the present disclosure includes the organic compound represented by Formula 1 according to the present disclosure, thereby improving the driving voltage, efficiency, and lifetime characteristics of the organic light emitting diode.

### CROSS-REFERENCE TO RELATED APPLICATION(S)

This application claims benefit of priority to Korean Patent Application No. 10-2024-0119102, filed on Sep. 3, 2024, and Korean Patent Application No. 10-2025-0110838, filed on Aug. 11, 2025, in the Korean Intellectual Property Office, the disclosures of which are incorporated herein by reference in their entireties.

### TECHNICAL FIELD

The present disclosure relates to an organic compound and an organic light emitting diode comprising the same.

### BACKGROUND

Organic light emitting diodes (OLEDs) have been actively developed and commercialized as light sources for flat panel displays such as wall-mountable televisions, backlights for displays, lighting devices, and signboards, because they have simplified structure, and various advantages in manufacturing processes, high luminance, excellent viewing angle characteristics, a fast response speed, and a low driving voltage, compared to other flat panel display devices such as conventional liquid crystal displays (LCDs), plasma display panels (PDPs), and field emission displays (FEDs).

The OLED comprises two electrodes and an organic layer disposed between the two electrodes. The OLED is an element that uses the principle that electrons and holes are injected into an emitting layer from the two electrodes, respectively, and are combined with each other in the emitting layer to generate excitons and light is generated when the generated excitons drop from an excited state to a ground state.

The OLED may comprise at least one emitting layer. In general, an OLED comprising a plurality of emitting layers may include emitting layers that emit light with different peak wavelengths, and implement a specific color may be implemented by a combination of light having the different peak wavelengths.

Such an OLED may be categorized into a top-emission structure and a bottom-emission structure. The top-emission OLED emits light generated in an emitting layer toward a translucent first electrode (anode) using a reflective second electrode (cathode). In contrast, the bottom-emission OLED emits light generated in an emitting layer and reflected by a reflective first electrode toward a transparent second electrode, which is a direction toward a driving thin film transistor, using the reflective first electrode.

### RELATED ART DOCUMENT

### [Patent Document]

(Patent Document 1) WO 2020-111253 A1 (Published: Jun. 4, 2020)
(Patent Document 2) KR 2017-0094665 A (Published: Aug. 21, 2017)

### SUMMARY

An object of the present disclosure is to provide a novel organic compound and an organic light emitting diode comprising the same.

Embodiments according to the present disclosure may be used to achieve other problems not specifically mentioned, in addition to the above problems.

The object of the present disclosure is not limited to those described above, and other objects and advantages of the present disclosure not mentioned, can be understood from the following description and will be more clearly understood from the embodiments of the present disclosure.

Furthermore, it will be readily apparent that the objects and advantages of the present disclosure may be realized by means and combinations thereof set forth in the claims.

To solve the above problems, according to an embodiment of the present disclosure, there is provided an organic compound represented by the following Chemical Formula 1:

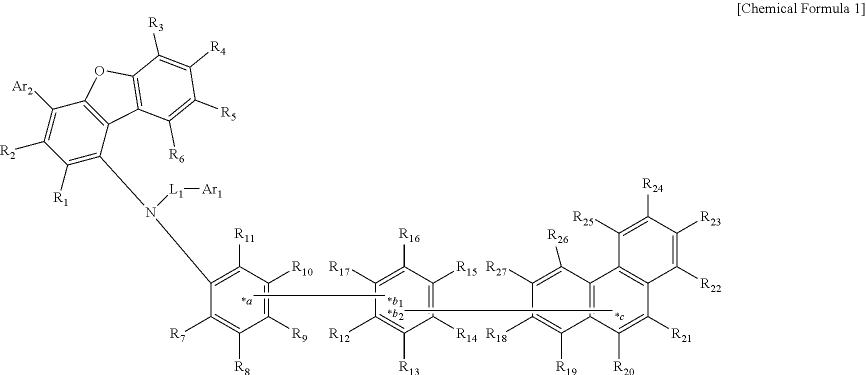

wherein in the chemical formula 1,
L₁ is selected from the group consisting of a single bond, a substituted or unsubstituted arylene group having 6 to 30 carbon atoms, and a substituted or unsubstituted heteroarylene group having 1 to 30 carbon atoms;
Ar₁ and Ar₂ are identical to or different from each other, and are each independently selected from the group consisting of a substituted or unsubstituted aryl group having 6 to 30 carbon atoms, and a substituted or unsubstituted heteroaryl group having 1 to 30 carbon atoms;
R₁ to R₂₇ are identical to or different from each other, and are each independently selected from hydrogen, deuterium, a substituted or unsubstituted alkyl group having 1 to 10 carbon atoms, a substituted or unsubstituted aryl group having 6 to 30 carbon atoms, and a substituted or unsubstituted heteroaryl group having 1 to 30 carbon atoms;
when L₁, Ar₁, Ar₂, and R₁ to R₂₇ are substituted, the substituents are identical to or different from each other, and may be one or more selected from the group consisting of deuterium, a cyano group, a nitro group, a halogen group, a hydroxy group, an alkyl group having 1 to 10 carbon atoms, a cycloalkyl group having 3 to 10 carbon atoms, an aryl group having 6 to 30 carbon atoms, a heteroaryl group having 1 to 30 carbon atoms, an alkylamino group having 1 to 10 carbon atoms, an arylamino group having 6 to 30 carbon atoms, a heteroarylamino group having 1 to 30 carbon atoms, an alkylsilyl group having 1 to 10 carbon atoms, an arylsilyl group having 6 to 30 carbon atoms, an alkoxy group having 1 to 10 carbon atoms, an aryloxy group having 6 to 30 carbon atoms, an alkylthio group having 1 to 10 carbon atoms, and an arylthio group having 6 to 30 carbon atoms;
*a, *b₁, and *b₂ are different from each other, and represent bonding positions on a phenylene moiety, and *c represents a bonding position on a phenanthryl moiety;
one of R₇ to Rn represents a single bond that is bonded to *a;
one of R₁₂ to R₁₇ represents a single bond that is bonded to *b₁;
one of R₁₂ to R₁₇ represents a single bond that is bonded to *b₂; and
one of R₁₈ to R₂₇ represents a single bond that is bonded to *c.

According to another embodiment of the present disclosure, there is provided an organic light emitting diode comprising: an anode; a cathode facing the anode; and one or more organic layers disposed between the anode and the cathode, wherein at least one of the organic layers includes the organic compound represented by Chemical Formula 1, and wherein the organic layer including the organic compound represented by Chemical Formula 1 is a hole transport layer or a hole transport auxiliary layer.

The organic compound represented by Chemical Formula 1 according to the present disclosure may exhibit excellent hole transport properties.

In addition, when the hole transport layer and/or a hole transport auxiliary layer incudes the organic compound represented by Chemical Formula 1 according to the present disclosure, the driving voltage, efficiency, and lifetime characteristics of the organic light emitting diode according to the present disclosure may be improved.

Furthermore, when the organic compound represented by Chemical Formula 1 according to the present disclosure is used as a material for the hole transport auxiliary layer, it may have an energy level suitable for serving as a hole transport auxiliary layer that transfers holes from the hole transport layer to the emitting layer and blocks electrons coming from the emitting layer.

Moreover, the organic light emitting diode according to the present disclosure may excellently realize a target color coordinates of the emitting layer, even when a hole transport layer and/or a hole transport auxiliary layer including the organic compound represented by Chemical Formula 1 according to the present disclosure is combined with an emitting layer of any color.

The effects of the present disclosure are not limited to those described above, and other effects not mentioned will be clearly understood by those skilled in the art from the following description.

### DETAILED DESCRIPTION

The aforementioned objects, features, and advantages will be described in detail below, and accordingly, those skilled in the art to which the present disclosure pertains will be able to readily practice the technical spirit of the present disclosure.

In the description of the present disclosure, detailed descriptions of known technologies related to the present disclosure will be omitted if they are deemed to unnecessarily obscure the gist of the present disclosure.

As used herein, it is to be understood that when expressions “comprising,” “having,” “consisting of,” “disposed,” “provided with,” etc. are used to describe components, additional components may be added, unless the term “only” is used. Also, it is to be understood that, unless expressly stated otherwise, a component referred to in the singular form may encompass the plural form as well.

In interpreting components in this specification, it is to be understood that the ranges include allowable tolerances even if not explicitly stated otherwise.

As used herein, it is to be understood that when any configuration is described as being disposed “on (or under)” a component or “on an upper portion (or lower portion)” of a component, any configuration may be disposed not only in contact with the top (or bottom) surface of the component, but also that other components may intervene between the component and any component disposed on (or under) the component.

As used herein, the term “halogen group” includes fluorine, chlorine, bromine, and iodine.

As used herein, the term “alkyl group” refers to both straight-chain alkyl radical and branched-chain alkyl radical. Unless otherwise specified, the alkyl group contains 1 to 10 carbon atoms and may include, but is not limited to, methyl, ethyl, propyl, isopropyl, butyl, sec-butyl, isobutyl, tert-butyl, pentyl, isoamyl, hexyl, etc. Additionally, the alkyl group may be optionally substituted.

As used herein, the term “cycloalkyl group” refers to a cyclic alkyl radical. Unless otherwise specified, the cycloalkyl group contains 3 to 10 carbon atoms and may include, but is not limited to, cyclopropyl, cyclobutyl, cyclopentyl, cyclohexyl, norbornyl, adamantyl, etc. Additionally, the cycloalkyl group may be optionally substituted.

As used herein, the term “alkenyl group” refers to both straight-chain alkenyl radical and branched-chain alkenyl radical having one or more carbon-carbon double bonds. Unless otherwise specified, the alkenyl group contains 2 to 30 carbon atoms and may include, but is not limited to, vinyl, allyl, isopropenyl, 2-butenyl, etc. Additionally, the alkenyl group may be optionally substituted.

As used herein, the term “cycloalkenyl group” refers to a cyclic alkenyl radical. Unless otherwise specified, the cycloalkenyl group contains 3 to 20 carbon atoms. Additionally, the cycloalkenyl group may be optionally substituted.

As used herein, the term “alkynyl group” refers to both straight-chain alkynyl radical and branched-chain alkynyl radical having one or more carbon-carbon triple bond. Unless otherwise specified, the alkynyl group contains 2 to 30 carbon atoms and may include, but is not limited to, ethynyl, 2-propynyl, etc. Additionally, the alkynyl group may be optionally substituted.

As used herein, the term “cycloalkynyl group” refers to a cyclic alkynyl radical. Unless otherwise specified, the cycloalkynyl group contains 3 to 20 carbon atoms. Additionally, the cycloalkynyl group may be optionally substituted.

As used herein, the term “aralkyl group” or “arylalkyl group” is used interchangeably, and refers to an alkyl group having an aromatic group as a substituent. Additionally, the aralkyl (arylalkyl) group may be optionally substituted.

As used herein, the term “aryl group” or “aromatic group” is used interchangeably, and the aryl group includes both monocyclic and fused ring groups. The fused ring may include two or more rings, wherein two carbon atoms are shared between two adjacent rings. It may also include structures in which two or more rings are simply linked or fused together. Unless otherwise specified, the aryl group contains 6 to 30 carbon atoms, and may include, but is not limited to, phenyl, naphthyl, anthracenyl, phenanthryl, fluorenyl, dimethylfluorenyl, diphenylfluorenyl, spirofluorenyl, etc. Additionally, the aryl group may be optionally substituted.

As used herein, the term “heteroaryl group” or “heteroaromatic group” is used interchangeably, and the heteroaryl group includes both monocyclic and fused ring groups. The fused ring may include two or more rings, wherein two carbon or heteroatom atoms are shared between two adjacent rings. It may also include structures in which two or more rings are simply linked or fused together. Unless otherwise specified, the heteroaryl group may contain 1 to 30 carbon atoms, and when the number of carbon atoms is one or two, ring may be formed by including additional heteroatoms. In addition, the heteroaryl group may contain 1 to 30 carbon atoms, wherein one or more carbon atoms in the ring are substituted with heteroatoms such as oxygen (O), nitrogen (N), sulfur (S), or selenium (Se). The heteroaryl group may include, but is not limited to, a 6-membered monocyclic ring such as pyridyl, pyrazinyl, pyrimidinyl, pyridazinyl, or triazinyl, a polycyclic ring such as phenoxathinyl, indolizinyl, indolyl, purinyl, quinolyl, isoquinolyl, benzoxyzolyl, benzothiazolyl, benzimidazolyl, benzofuranyl, dibenzofuranyl, benzothiophenyl, dibenzothiophenyl, phenylcarbazolyl, 9-phenylcarbazolyl, or carbazolyl, and 2-furanyl, N-imidazolyl, 2-isoxazolyl, 2-pyridyl, or 2-pyrimidyl. Additionally, the heteroaryl group may be optionally substituted.

As used herein, the term “heterocyclic group” means that one or more carbon atoms of an aryl group, a cycloalkyl group, a cycloalkenyl group, a cycloalkynyl group, an arylalkyl group, or an arylamino group are replaced with a heteroatom such as oxygen (O), nitrogen (N), sulfur (S), or selenium (Se). Based on the above definition, the heterocyclic group includes a heteroaryl group, a heterocycloalkyl group, a heterocycloalkenyl group, a heterocycloalkynyl group, a heteroarylalkyl group, and a heteroarylamino group. Additionally, the heterocyclic group may be optionally substituted.

As used herein, the term “carbon ring” may be used as a term including both “cycloalkyl group,” which is an alicyclic ring group, and “aryl group (aromatic group),” which is an aromatic ring group, unless otherwise specified.

As used herein, the terms “heteroalkyl group” and “heteroarylalkyl group” mean that one or more carbon atoms of an alkyl group or an arylalkyl group are replaced with a heteroatom such as oxygen (O), nitrogen (N), sulfur (S), or selenium (Se). Additionally, the heteroalkyl group and the heteroarylalkyl group may be optionally substituted.

As used herein, the terms “alkylamino group,” “arylalkylamino group,” “arylamino group,” and “heteroarylamino group” mean that at least one hydrogen atom of amino groups (or amine groups) is replaced with an alkyl group, an arylalkyl group, an aryl group, or an heteroaryl group, and the terms include all primary, secondary, and tertiary amino groups (or amine groups). Additionally, the alkylamino group, arylalkylamino group, arylamino group, and heteroarylamino group may be optionally substituted.

As used herein, the terms “alkylsilyl group,” “arylsilyl group,” “alkoxy group,” “aryloxy group,” “alkylthio group,” and “arylthio group” mean that a silyl group, an oxy group, and a thio group are respectively substituted with the alkyl group and aryl group as described above. Additionally, the alkylsilyl group, the arylsilyl group, the alkoxy group, the aryloxy group, the alkylthio group, and arylthio group may be optionally substituted.

As used herein, the terms “arylene group,” “arylalkylene group,” “heteroarylene group,” and “heteroarylalkylene group” refer to a divalent substituent derived from an aryl group, an arylalkyl group, an heteroaryl group, and a heteroarylalkyl group, respectively, each of which includes one additional substitution. Additionally, the arylene group, arylalkylene group, heteroarylene group, and heteroarylalkylene group may be optionally substituted.

As used herein, the term “substitution” means that a hydrogen (H) atom bonded to a carbon atom in the compound of the present disclosure is replaced with a substituent other than hydrogen. When multiple substituents are present, each substituent may be identical to or different from each other.

The substituents may each independently be selected from the group consisting of deuterium, a cyano group, a nitro group, a halogen group, a hydroxy group, an alkyl group having 1 to 10 carbon atoms, a cycloalkyl group having 3 to 10 carbon atoms, an aryl group having 6 to 30 carbon atoms, a heteroaryl group having 1 to 30 carbon atoms, an alkylamino group having 1 to 10 carbon atoms, an arylamino group having 6 to 30 carbon atoms, a heteroarylamino group having 1 to 30 carbon atoms, an alkylsilyl group having 1 to 10 carbon atoms, an arylsilyl group having 6 to 30 carbon atoms, an alkoxy group having 1 to 10 carbon atoms, an aryloxy group having 6 to 30 carbon atoms, an alkylthio group having 1 to 10 carbon atoms, and an arylthio group having 6 to 30 carbon atoms.

Each target and substituent defined herein may be identical to or different from each other, unless otherwise specified.

The unit used herein are based on weight (wt), unless otherwise specified. For example, when “%” is indicated, it shall be interpreted as weight percent (wt %).

Hereinafter, an organic compound according to the present disclosure and an organic light emitting diode comprising the same will be described in detail.

According to an embodiment of the present disclosure, L₁ in Chemical Formula 1 may be a single bond, or a substituted or unsubstituted arylene group having 6 to 15 carbon atoms. For example, L₁ may be a single bond, or a substituted or unsubstituted phenyl.

According to an embodiment of the present disclosure, Ar₁ in Chemical Formula 1 may be a substituted or unsubstituted aryl group having 6 to 30 carbon atoms, or a substituted or unsubstituted heteroaryl group having 1 to 30 carbon atoms, wherein the substituents, if any, contain at least one heteroatom selected from oxygen (O), sulfur (S), and nitrogen (N). For example, Ar₁ may be selected from the group consisting of substituted or unsubstituted phenyl, substituted or unsubstituted naphthyl, substituted or unsubstituted phenanthryl, substituted or unsubstituted dibenzofuranyl, substituted or unsubstituted dibenzothiophenyl, substituted or unsubstituted carbazolyl, and substituted or unsubstituted dimethylfluorenyl.

According to an embodiment of the present disclosure, Ar₂ in Chemical Formula 1 may be a substituted or unsubstituted aryl group having 6 to 15 carbon atoms. For example, Ar₂ may be selected from the group consisting of substituted or unsubstituted phenyl, substituted or unsubstituted naphthyl, and substituted or unsubstituted phenanthryl.

According to an embodiment of the present disclosure, the substituents of L₁, Ar₁, Ar₂, and R₁ to R₂₇ in Chemical Formula 1 are identical to or different from each other, and may each independently be one or more selected from the group consisting of deuterium, methyl, ethyl, propyl, butyl, pentyl, hexyl, cyclopropyl, cyclobutyl, cyclopentyl, cyclohexyl, adamantyl, phenyl, biphenyl, naphthyl, phenyl-naphthyl, anthracenyl, phenanthryl, dimethylfluorenyl, diphenylfluorenyl, spirofluorenyl, benzofuranyl, benzothiophenyl, dibenzofuranyl, dibenzothiophenyl, carbazolyl, phenylcarbazolyl, and 9-phenylcarbazolyl.

According to an embodiment of the present disclosure, Ar₂ in Chemical Formula 1 may be any one of the following Chemical Formulas 2 to 5:

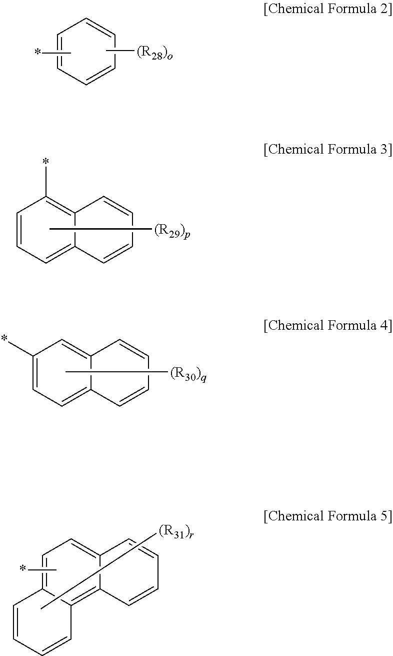

wherein in the chemical formulas 2 to 5,
R₂₈ to R₃₁ are identical to or different from each other, and are each independently one selected from the group consisting of hydrogen, deuterium, a substituted or unsubstituted alkyl group having 1 to 10 carbon atoms, a substituted or unsubstituted aryl group having 6 to 30 carbon atoms, and a substituted or unsubstituted heteroaryl group having 1 to 30 carbon atoms;
o is an integer from 1 to 5; each of p and q is an integer from 1 to 7; and r is an integer from 1 to 9; and
* denotes a bonding position.

According to an embodiment of the present disclosure, Chemical Formula 1 may be represented by any one of the following Chemical Formulas 6 to 8:

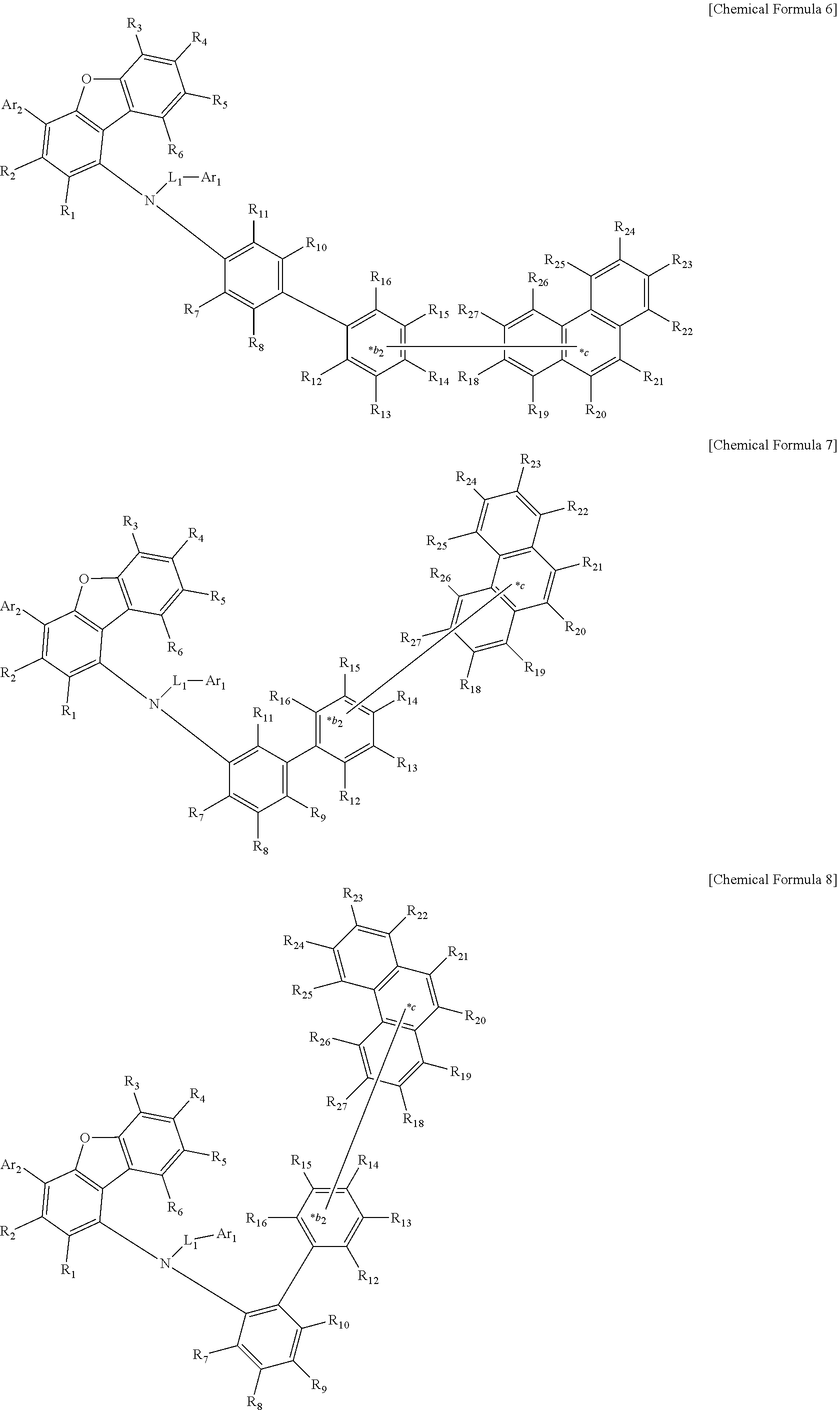

wherein in the chemical formulas 6 to 8,
L₁, Ar₁, Ar₂, and R₁ to R₂₇, and the definitions of the substituents thereof are as defined in Chemical Formula 1.
*b₂ represents a bonding position on a phenylene moiety, and *c represents a bonding position on a phenanthryl moiety; and
one of R₁₂ to R₁₆ represents a single bond that is boned to *b₂, and one of R₁₈ to R₂₇ represents a single bond that is boned to *c.

For example, R₁₄ present at the position denoted by *b₂ may be bonded via a single bond to R₂₁ present at the position denoted by *c.

According to an embodiment of the present disclosure, Chemical Formula 1 may be represented by any one of the following Chemical Formulas 9 to 17:

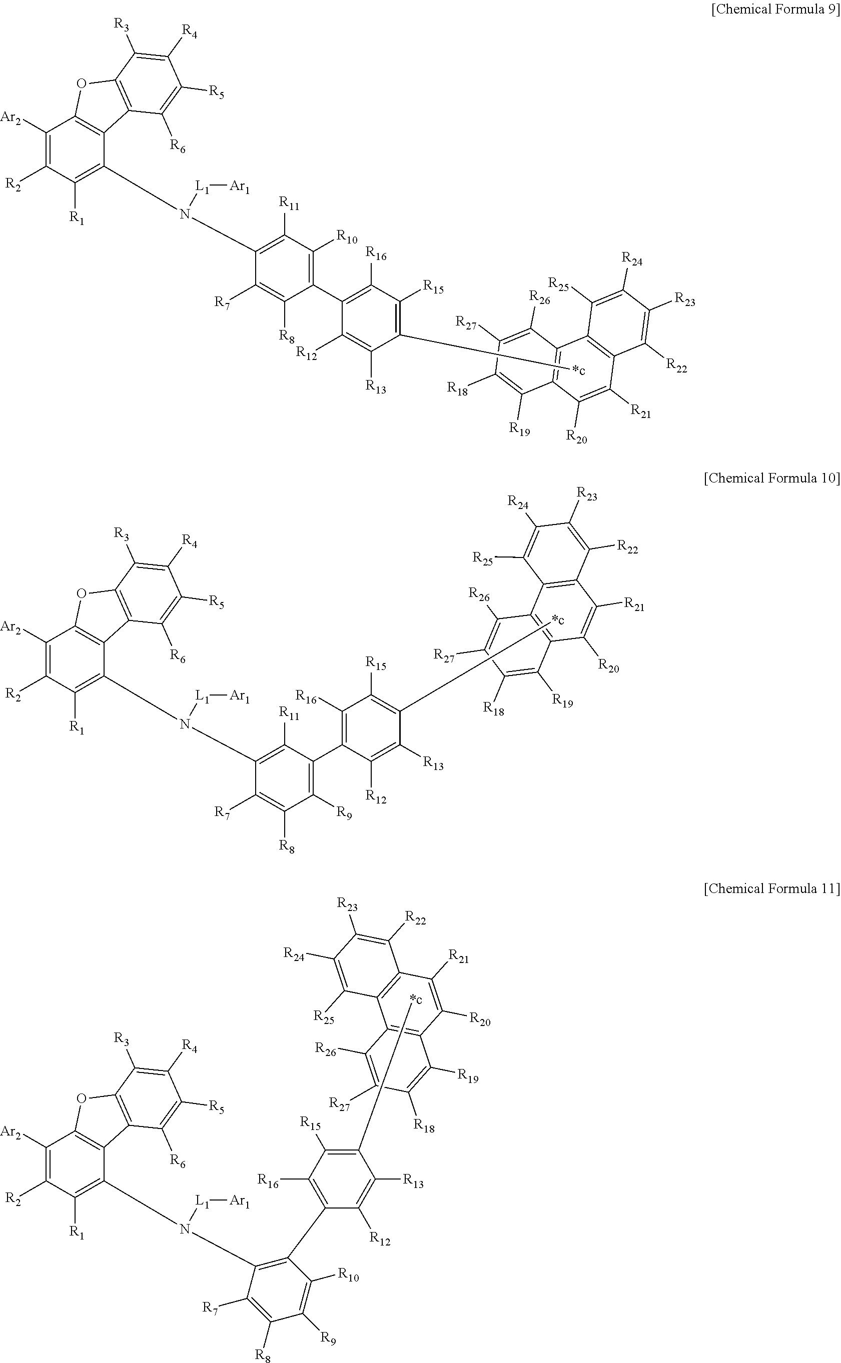

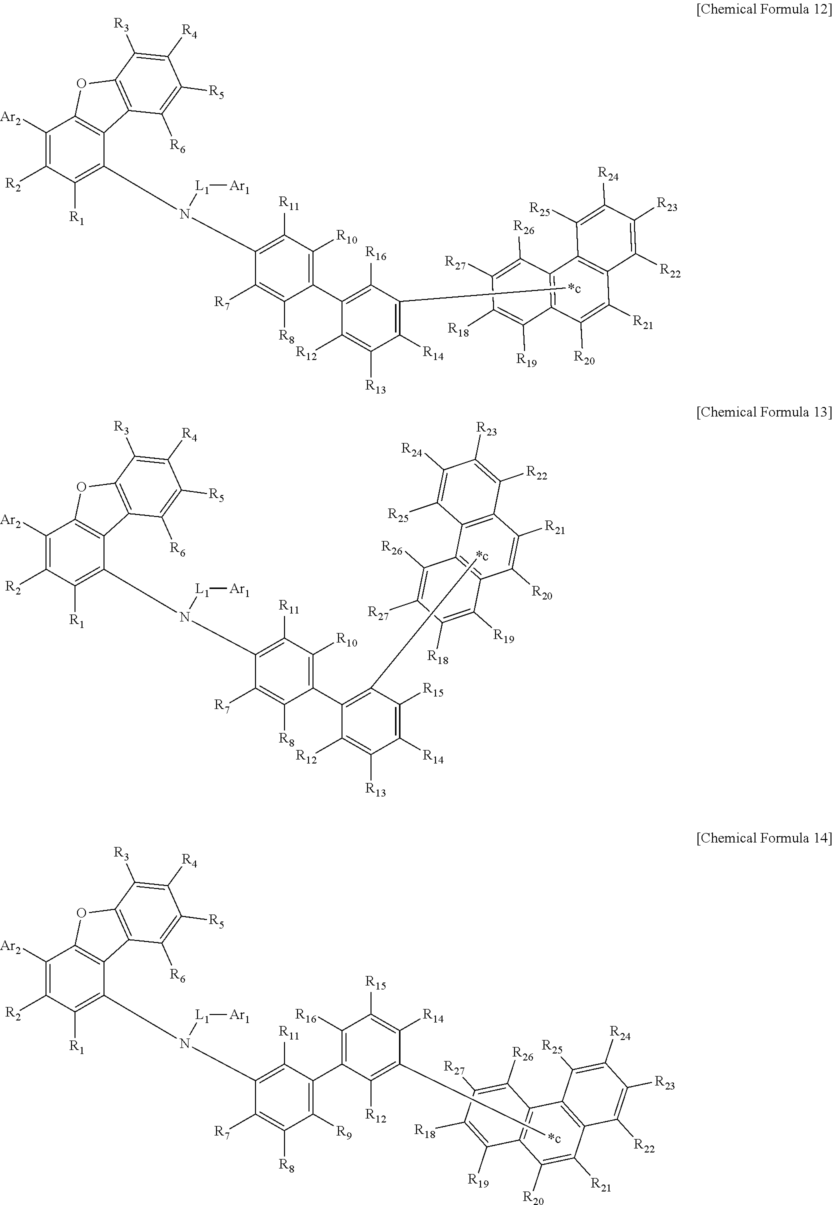

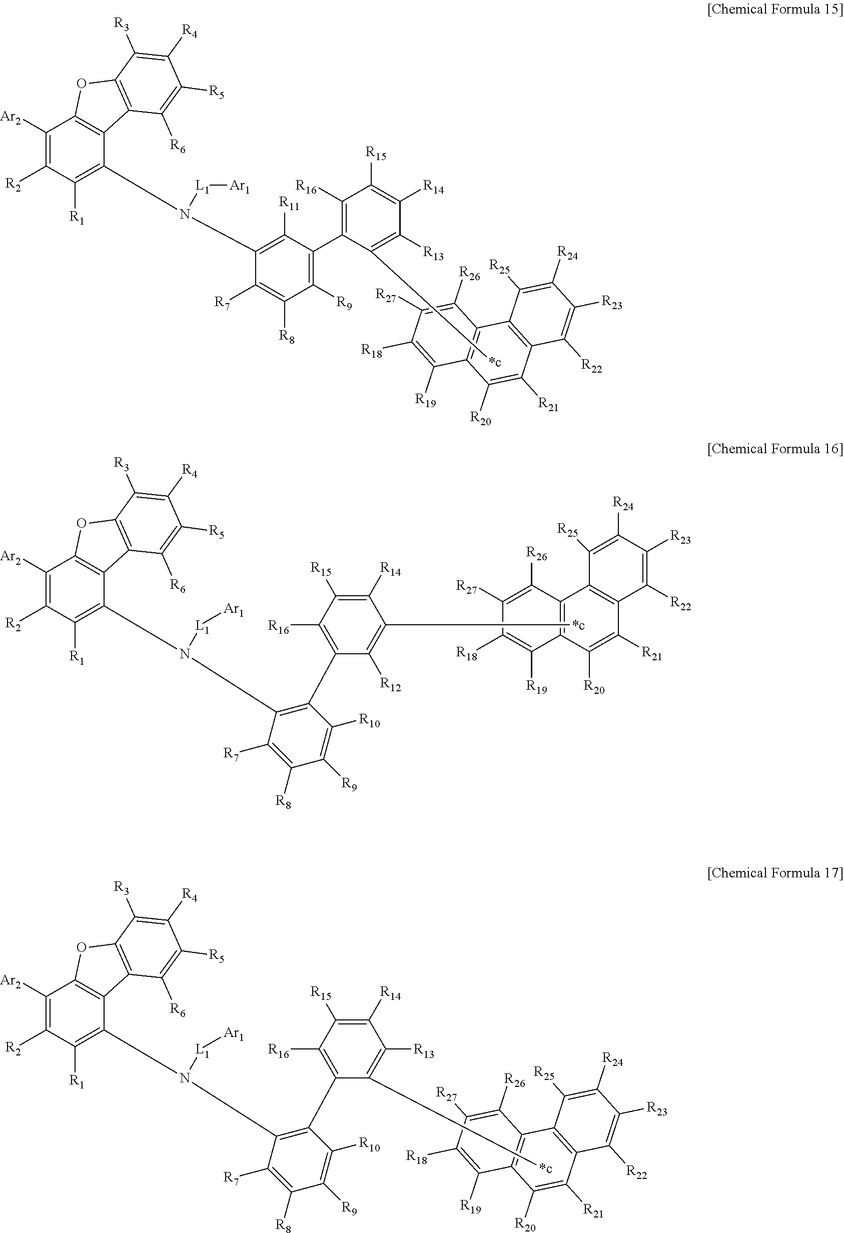

wherein in the chemical formulas 9 to 17,
L₁, Ar₁, Ar₂, and R₁ to R₂₇, and the definitions of the substituents thereof are as defined in Chemical Formula 1.
*c represents a bonding position on a phenylene moiety, and one of R₁₈ to R₂₇ represents a single bond that is bonded to the *c.

According to an embodiment of the present disclosure, Ar₁ may be selected from any one of the following Chemical Formulas 18 to 21:

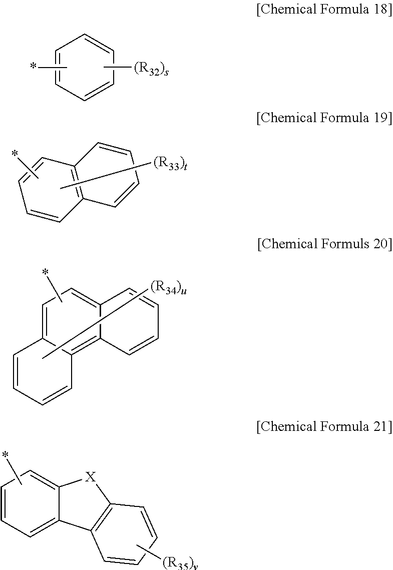

wherein in the chemical formulas 18 to 21,
R₃₂ to R₃₅ are identical to or different from each other, and are each independently one selected from the group consisting of hydrogen, deuterium, a substituted or unsubstituted alkyl group having 1 to 10 carbon atoms, a substituted or unsubstituted aryl group having 6 to 30 carbon atoms, and a substituted or unsubstituted heteroaryl group having 1 to 30 carbon atoms;
s is an integer from 1 to 5; each of t and v is integer from 1 to 7; and u is an integer from 1 to 9.

In Chemical Formula 21, x may be any one selected from the group consisting of O, S, NR₃₆, and CR₃₇R₃₈.

R₃₆ to R₃₈ are identical to or different from each other, and are each independently one selected from the group consisting of hydrogen, deuterium, a substituted or unsubstituted alkyl group having 1 to 10 carbon atoms, a substituted or unsubstituted aryl group having 6 to 30 carbon atoms, and a substituted or unsubstituted heteroaryl group having 1 to 30 carbon atoms.
* denotes a bonding position.

According to an embodiment of the present disclosure, the compound of the present disclosure may include, for example, a phenyl group, a naphthyl group, or a phenanthryl group at the 4-position of a dibenzofuran moiety, and may further include a phenanthryl group bonded to the nitrogen atom of an amine compound via a p-biphenylene linker. Accordingly, such compounds may exhibit a more extended conjugated system (or delocalized bond) compared to compounds not comprising such structural features, thereby achieving improved stability and higher efficiency.

However, a compound comprising a low-molecular-weight structure such as a phenyl or naphthyl group at the 4-position of the dibenzofuran moiety may exhibit higher thermal stability than a compound comprising a relatively high-molecular-weight phenanthrene structures at the same position.

By having an asymmetric structure centered on the arylamine moiety, the HOMO-LUMO bandgap may be readily adjusted, and the crystallinity of the molecule may be reduced due to the asymmetry. Low crystallinity not only facilitates the purification of the compound to obtain a high-purity product, but also reduces the risk of clogging due to material condensation at the inlet of the container during OLED manufacturing processes such as deposition.

According to an embodiment of the present disclosure, the organic compound represented by Chemical Formula 1 may be selected from the group consisting of, but is not limited to, the following compounds.

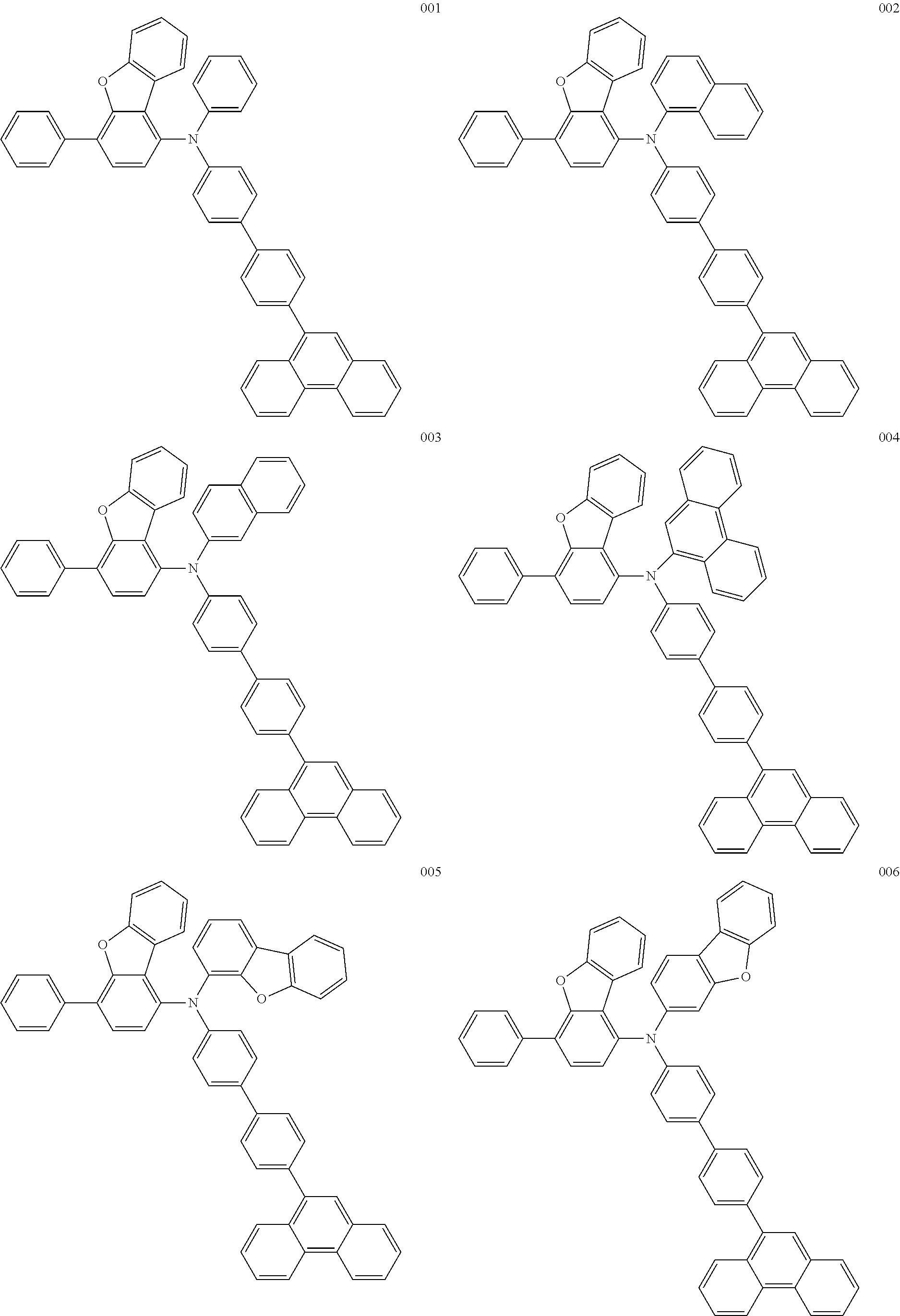

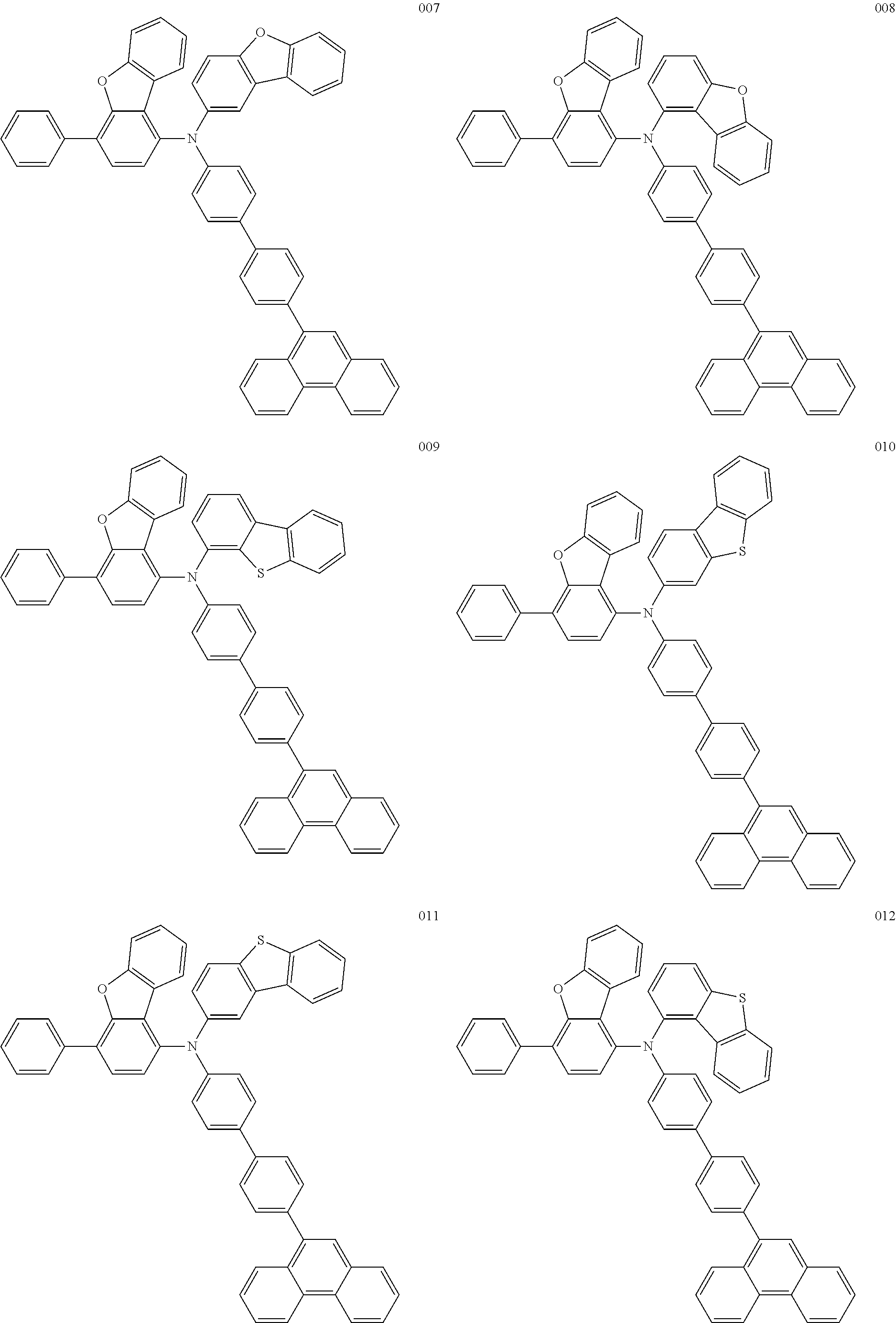

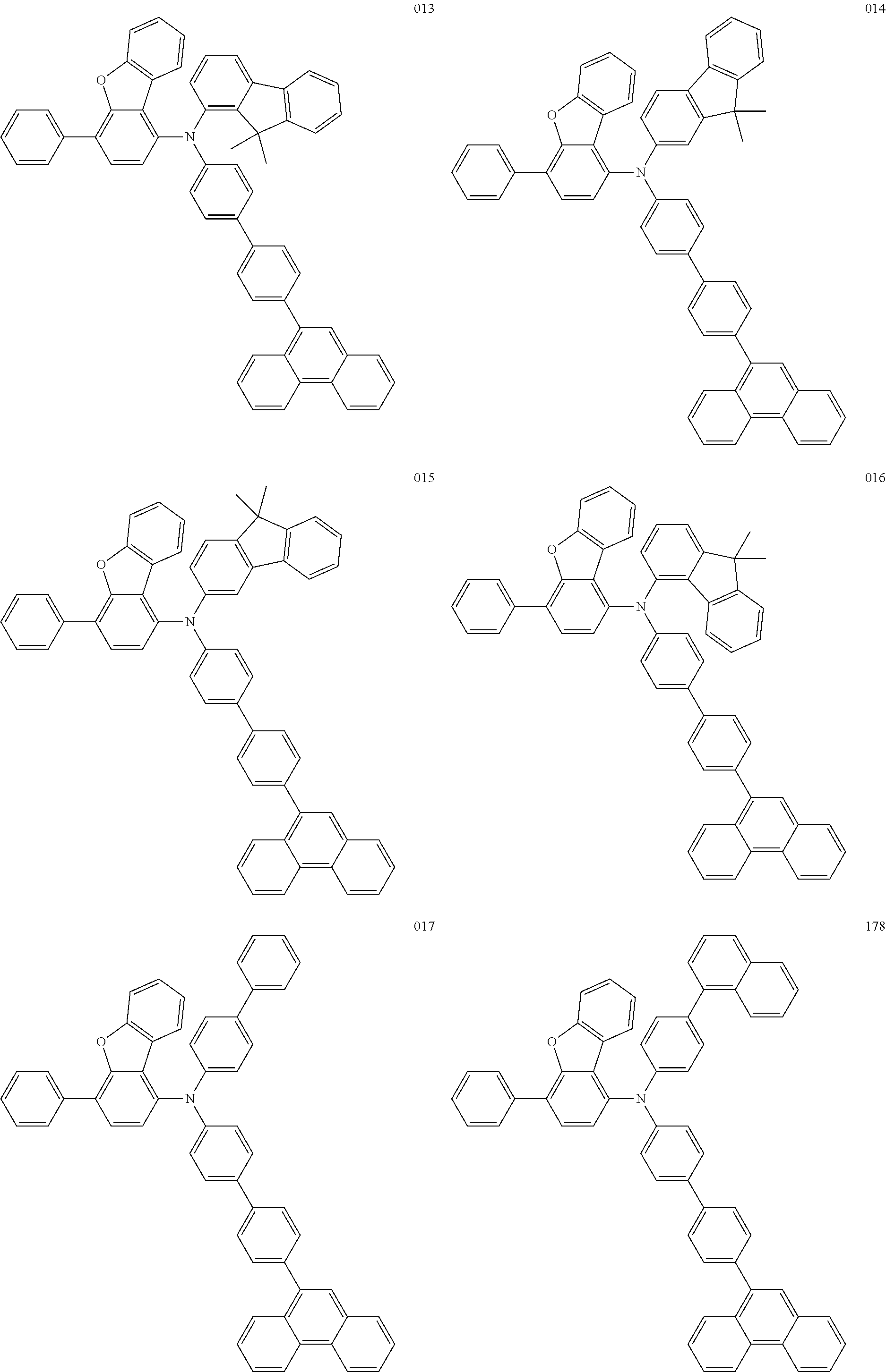

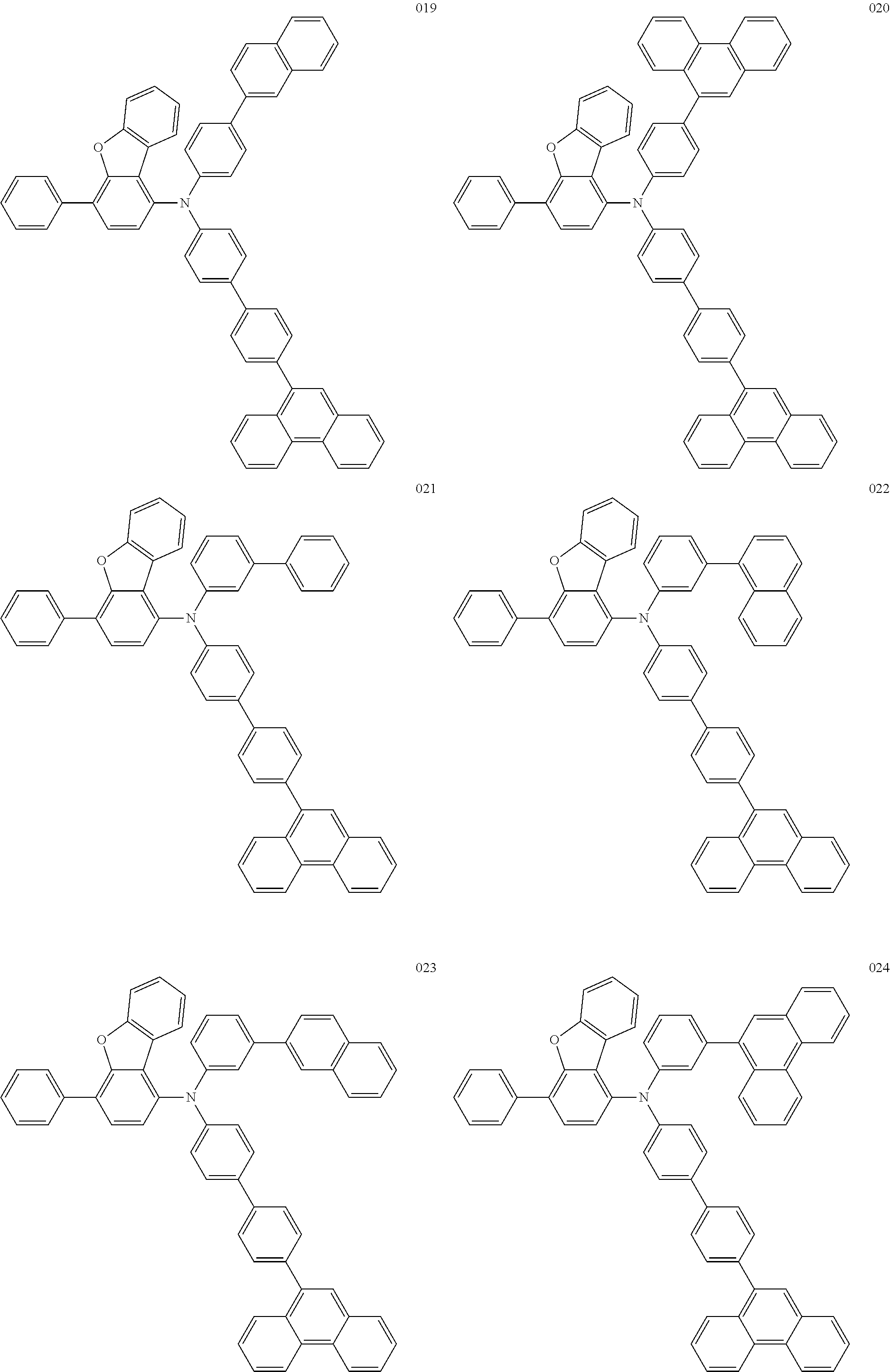

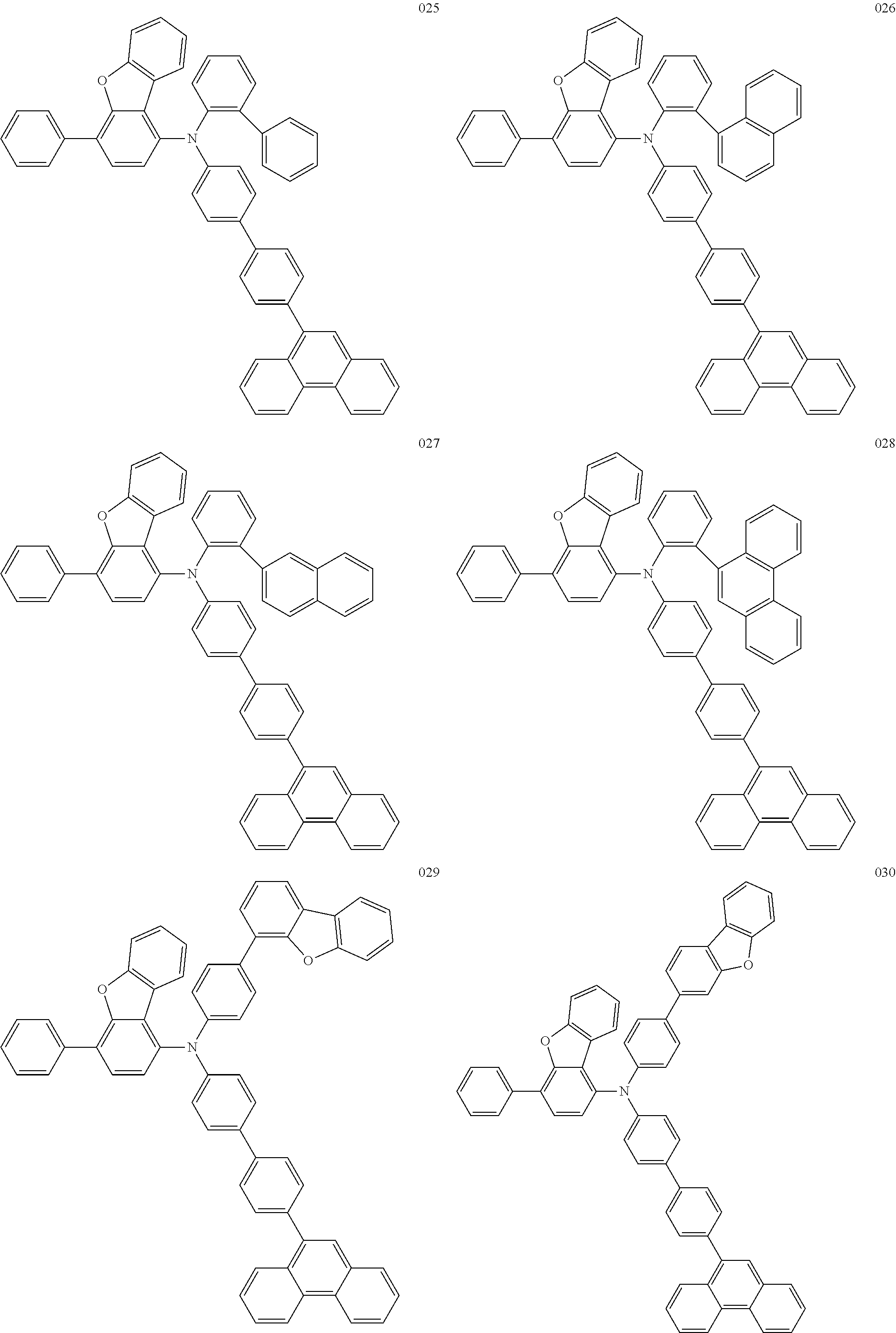

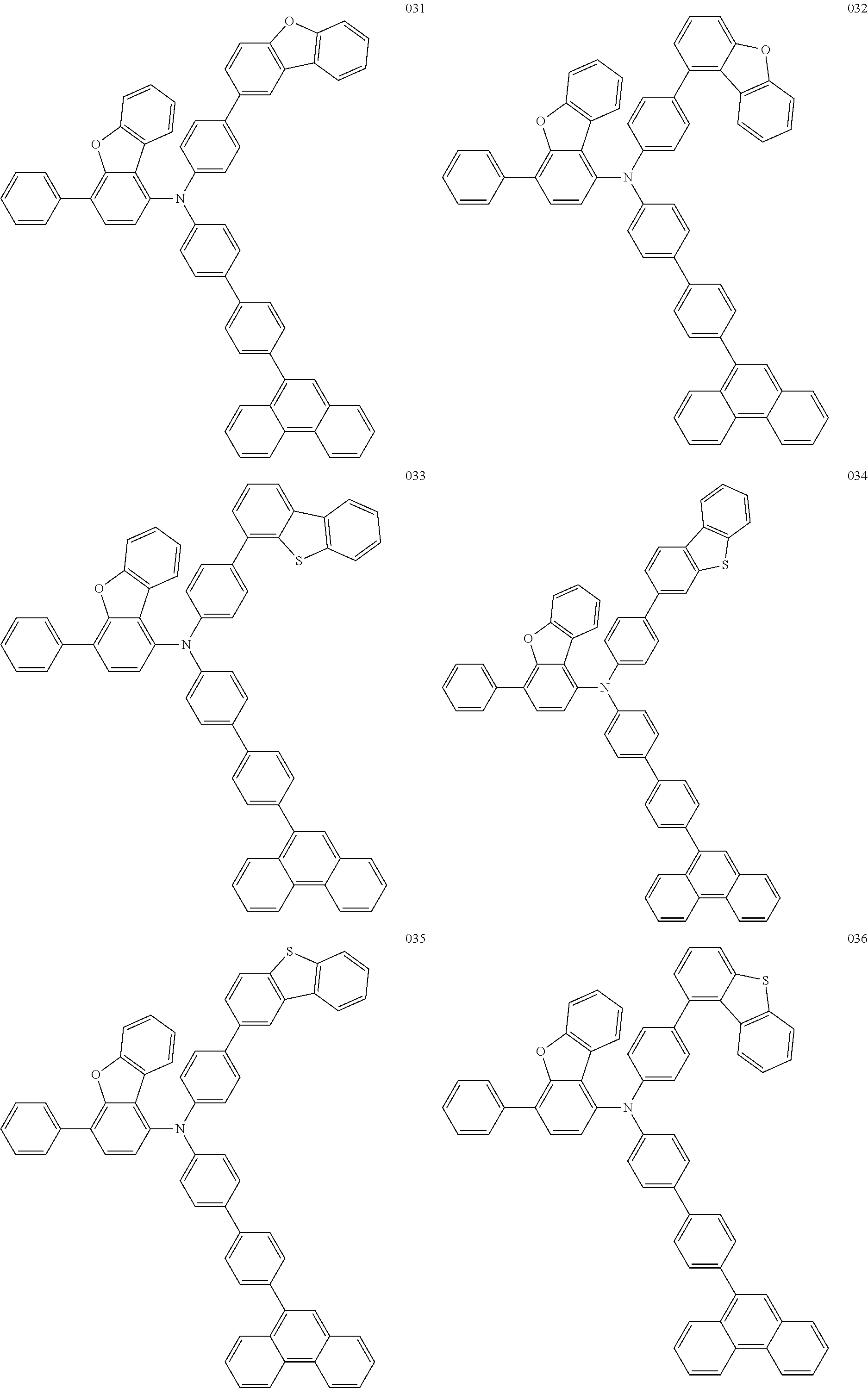

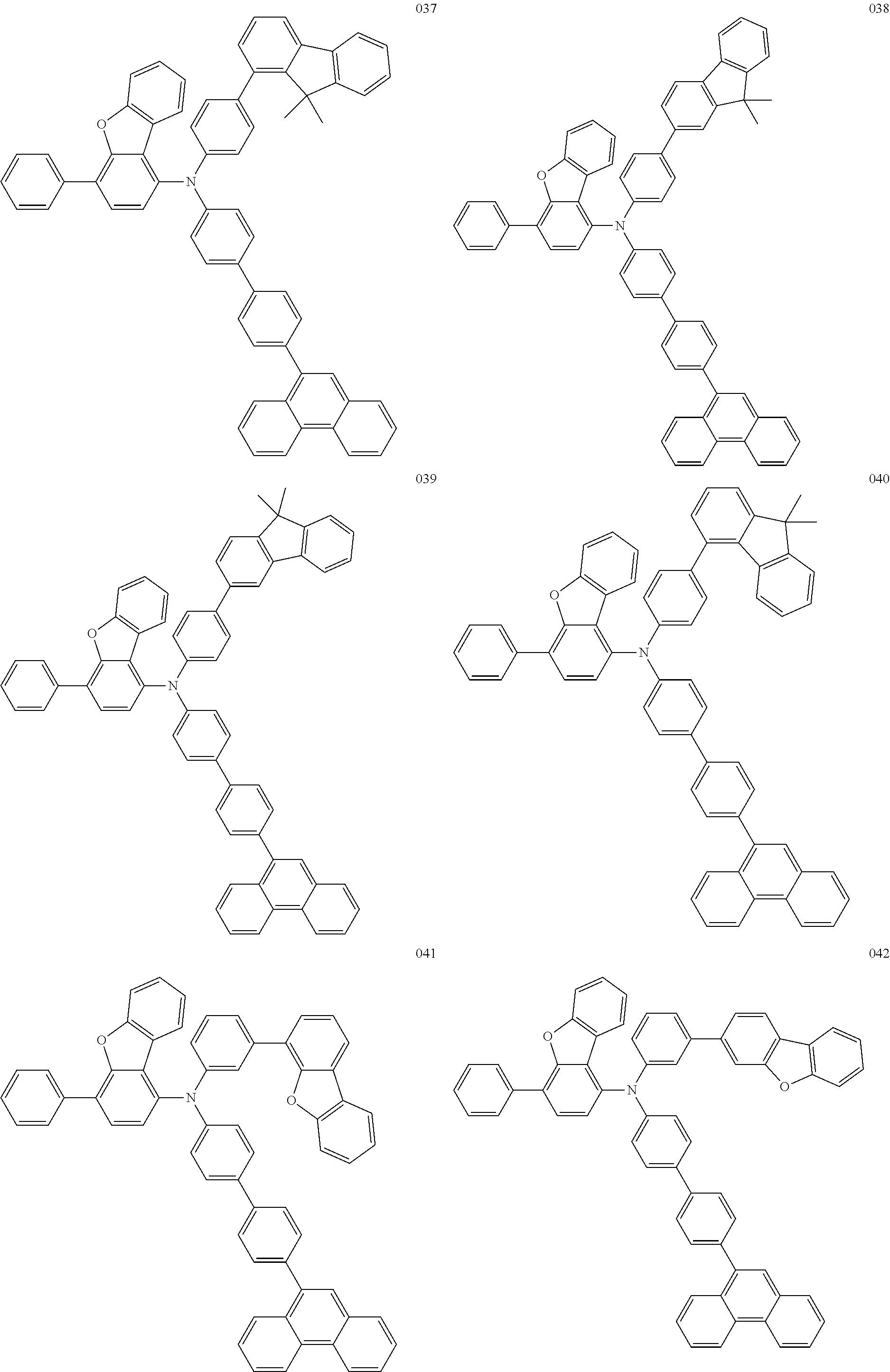

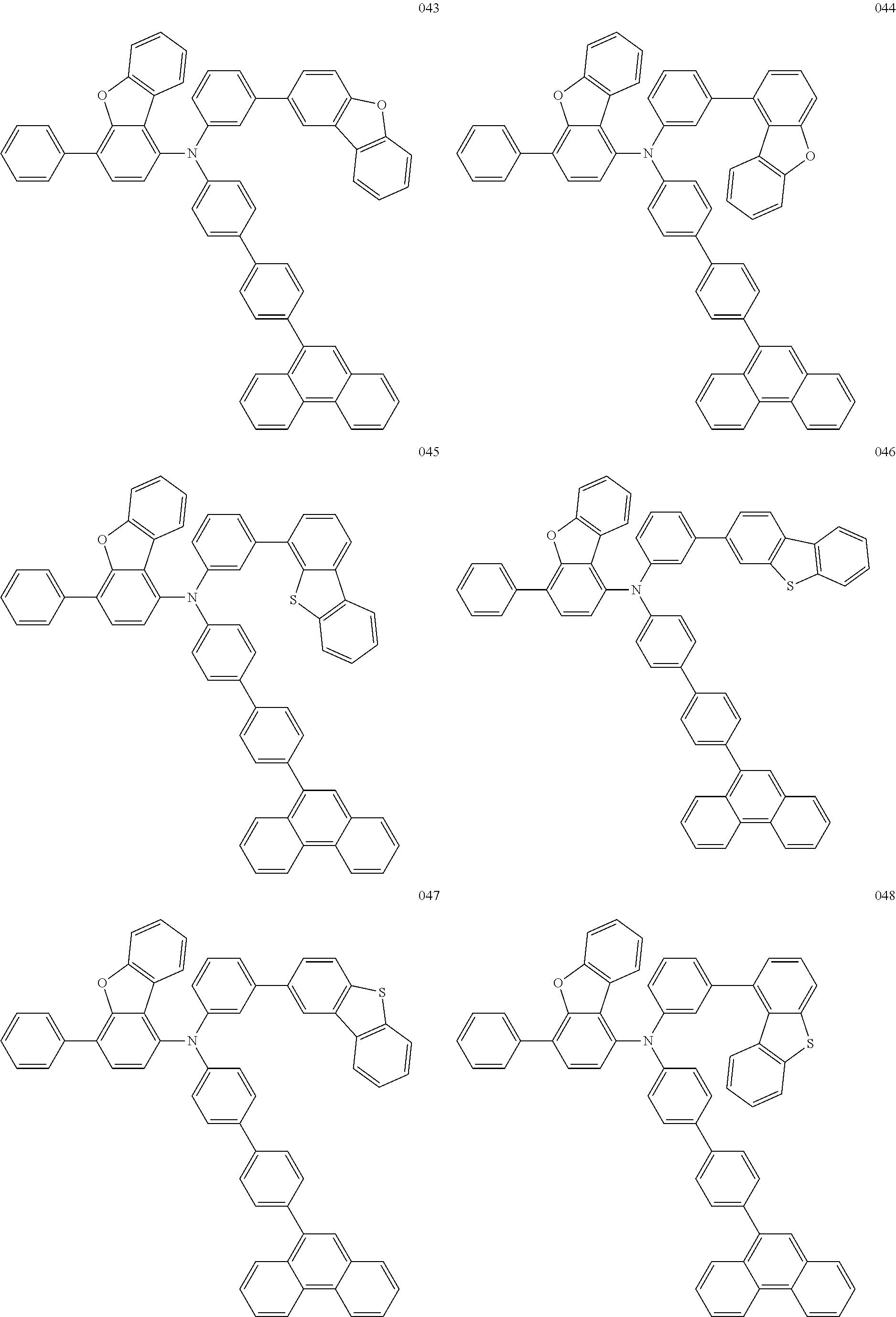

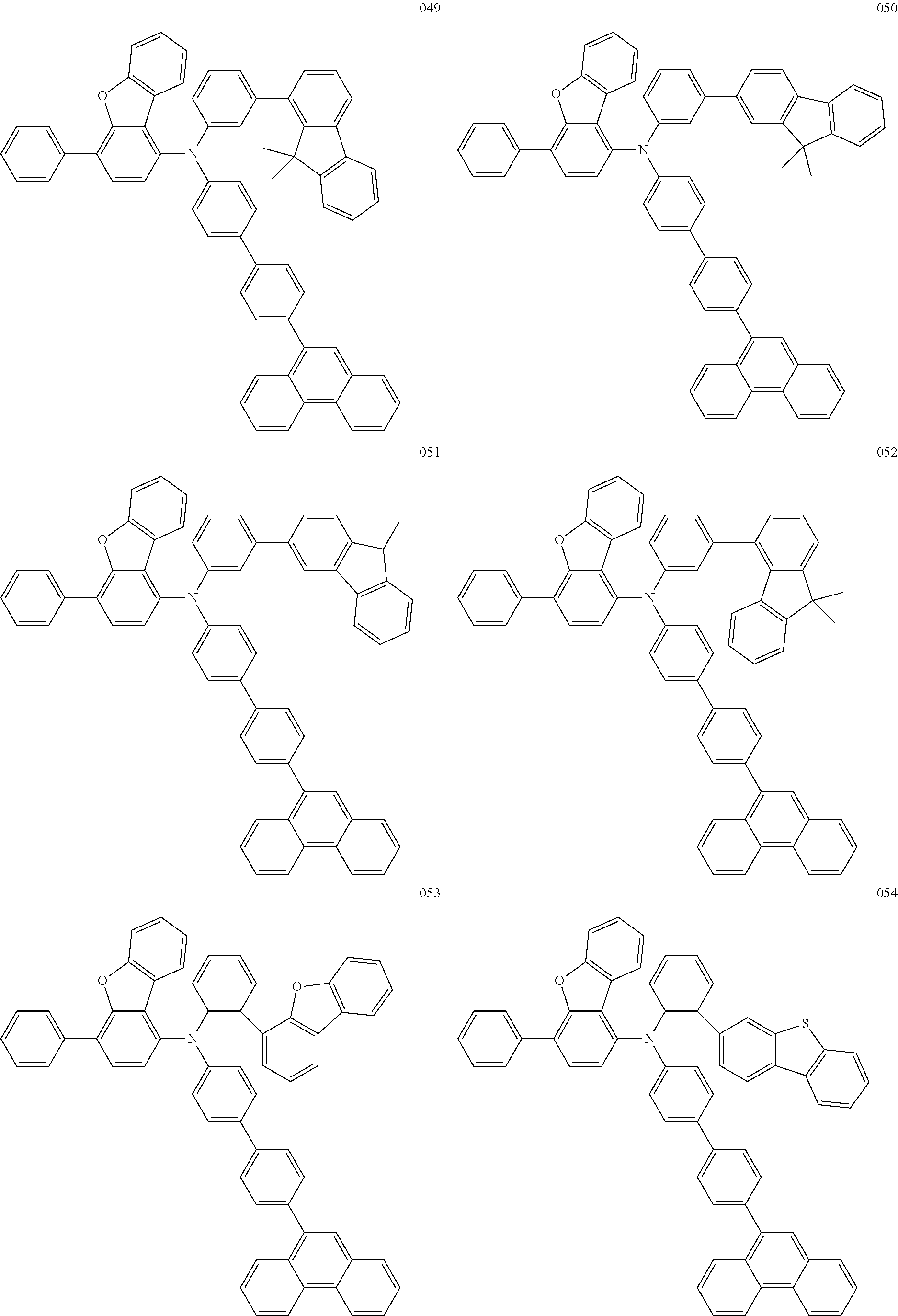

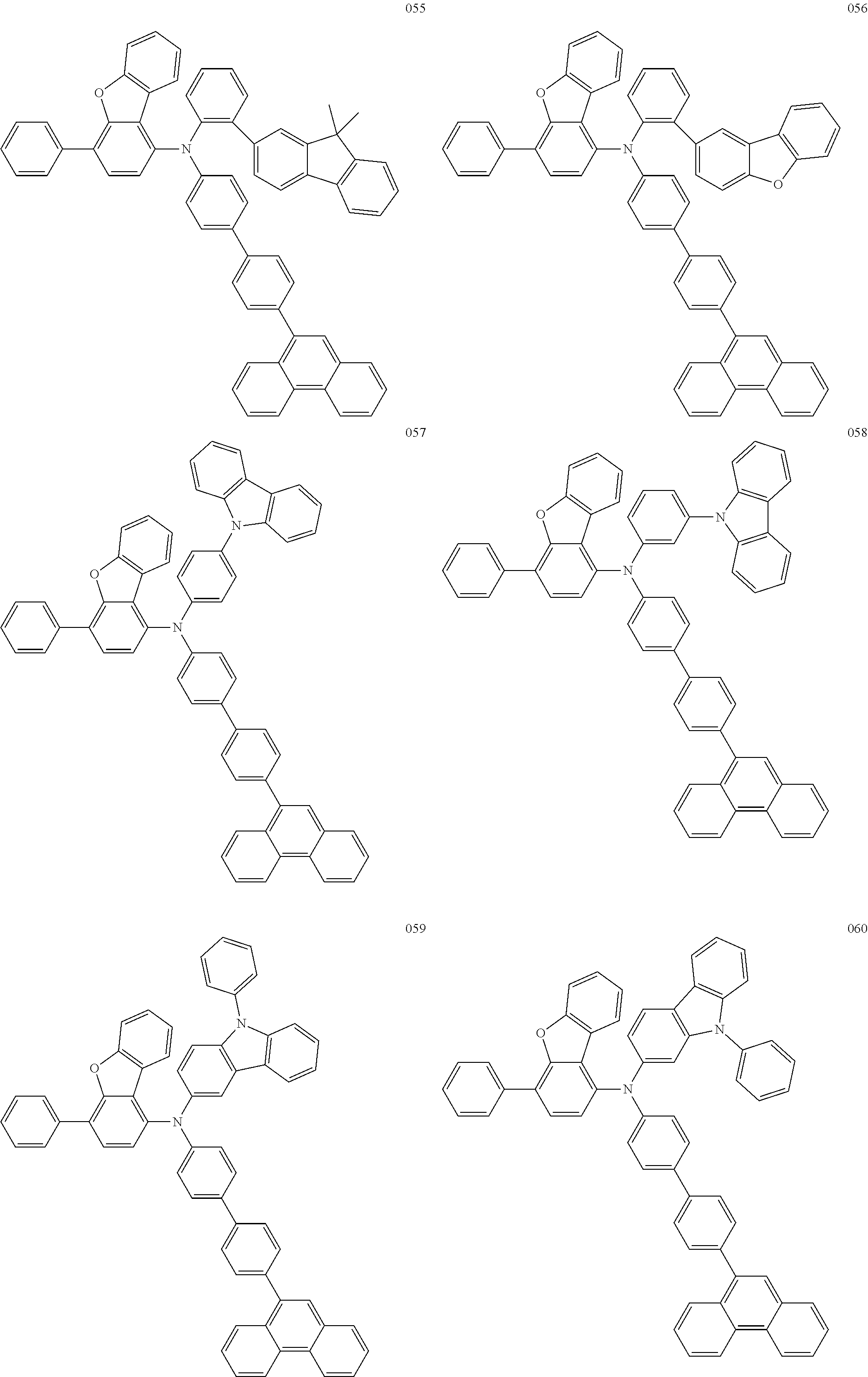

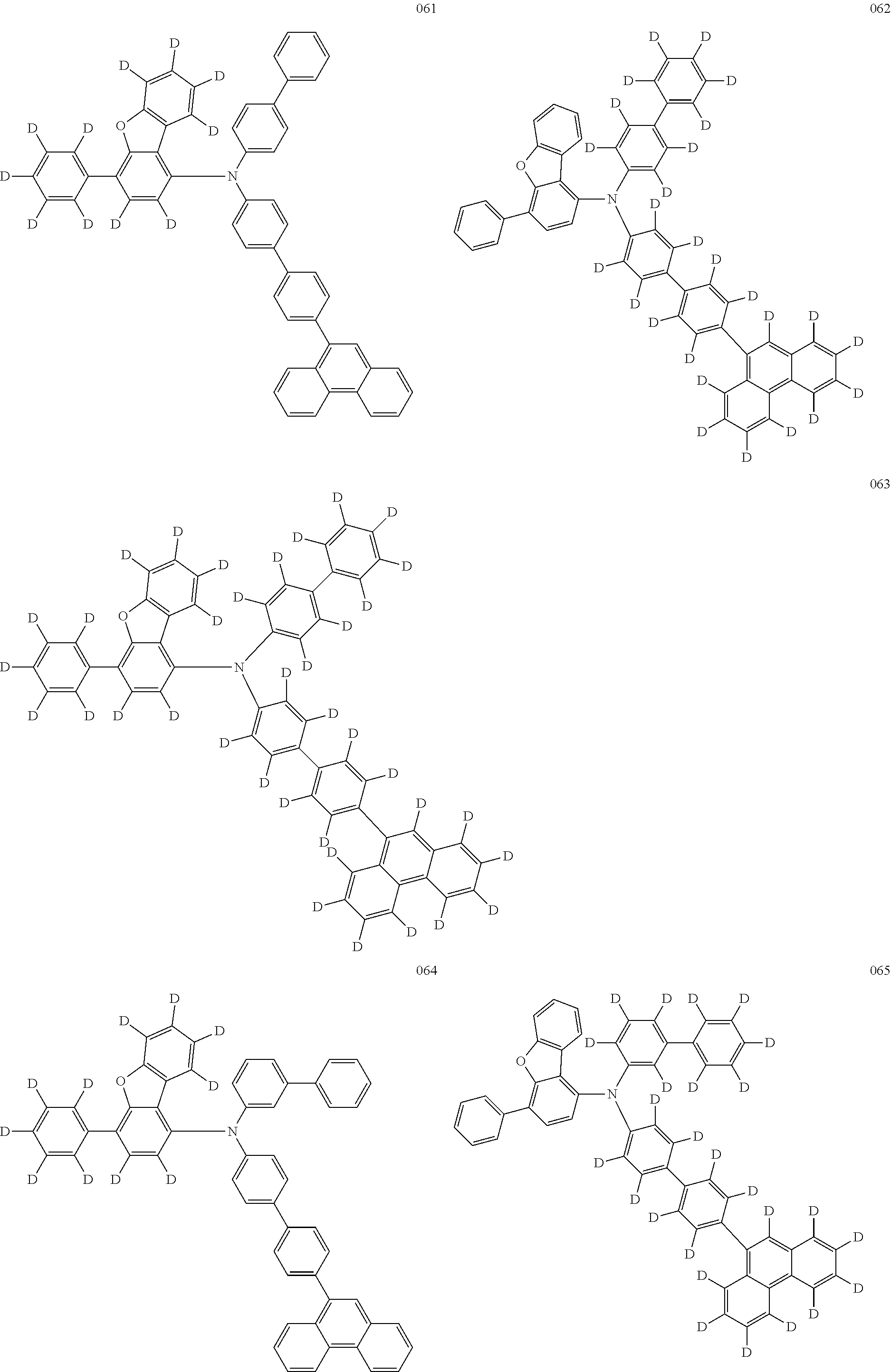

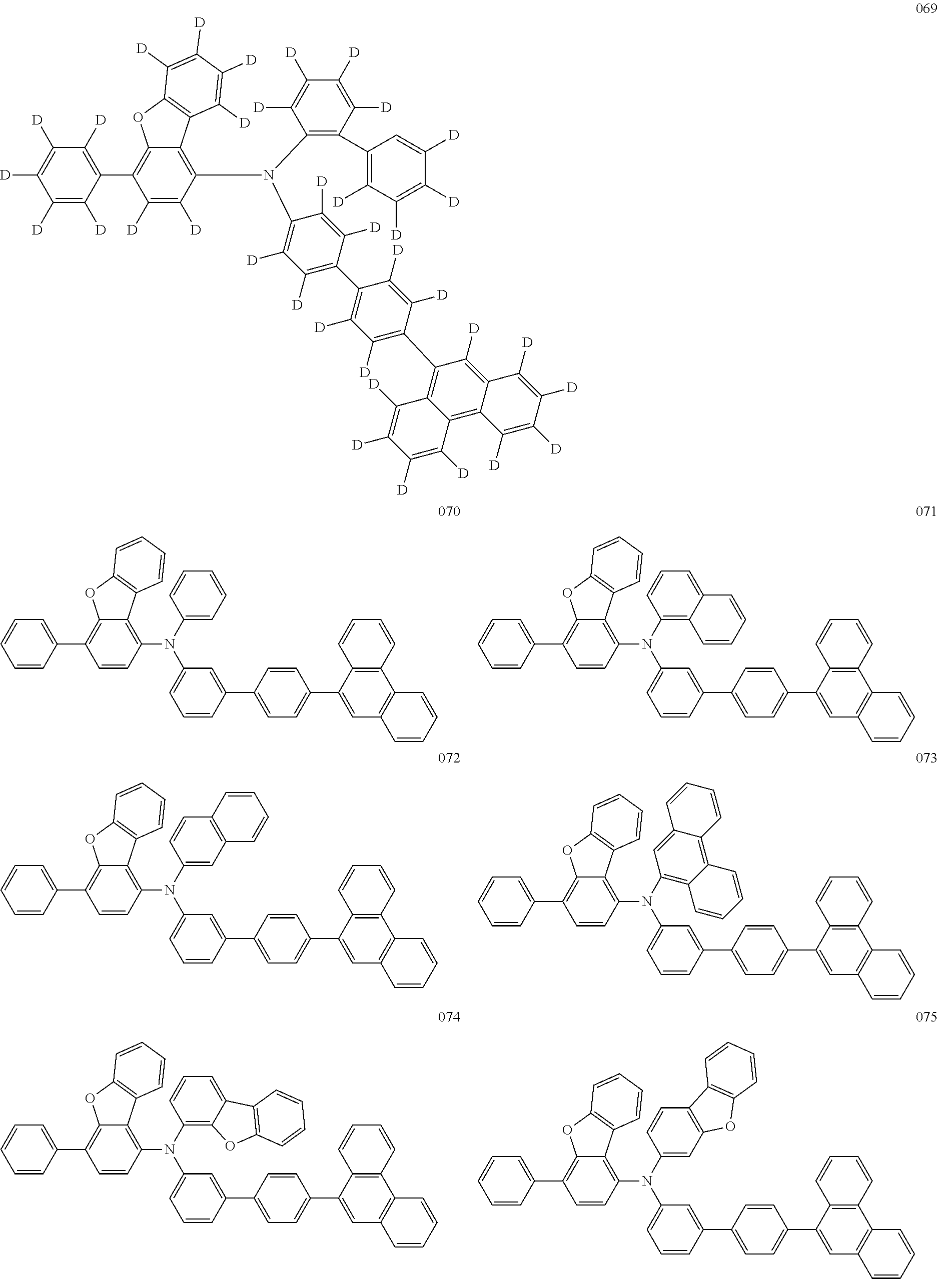

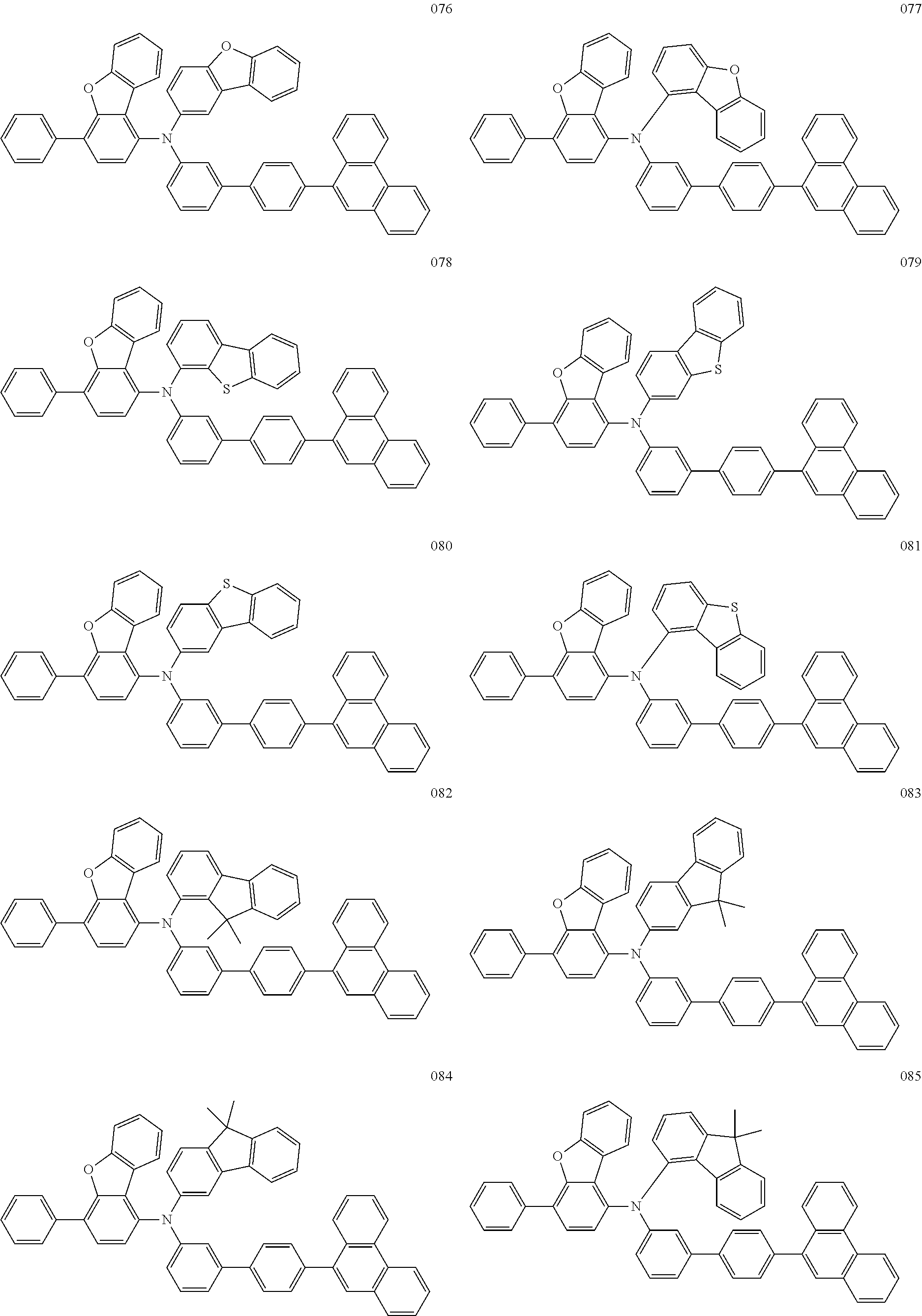

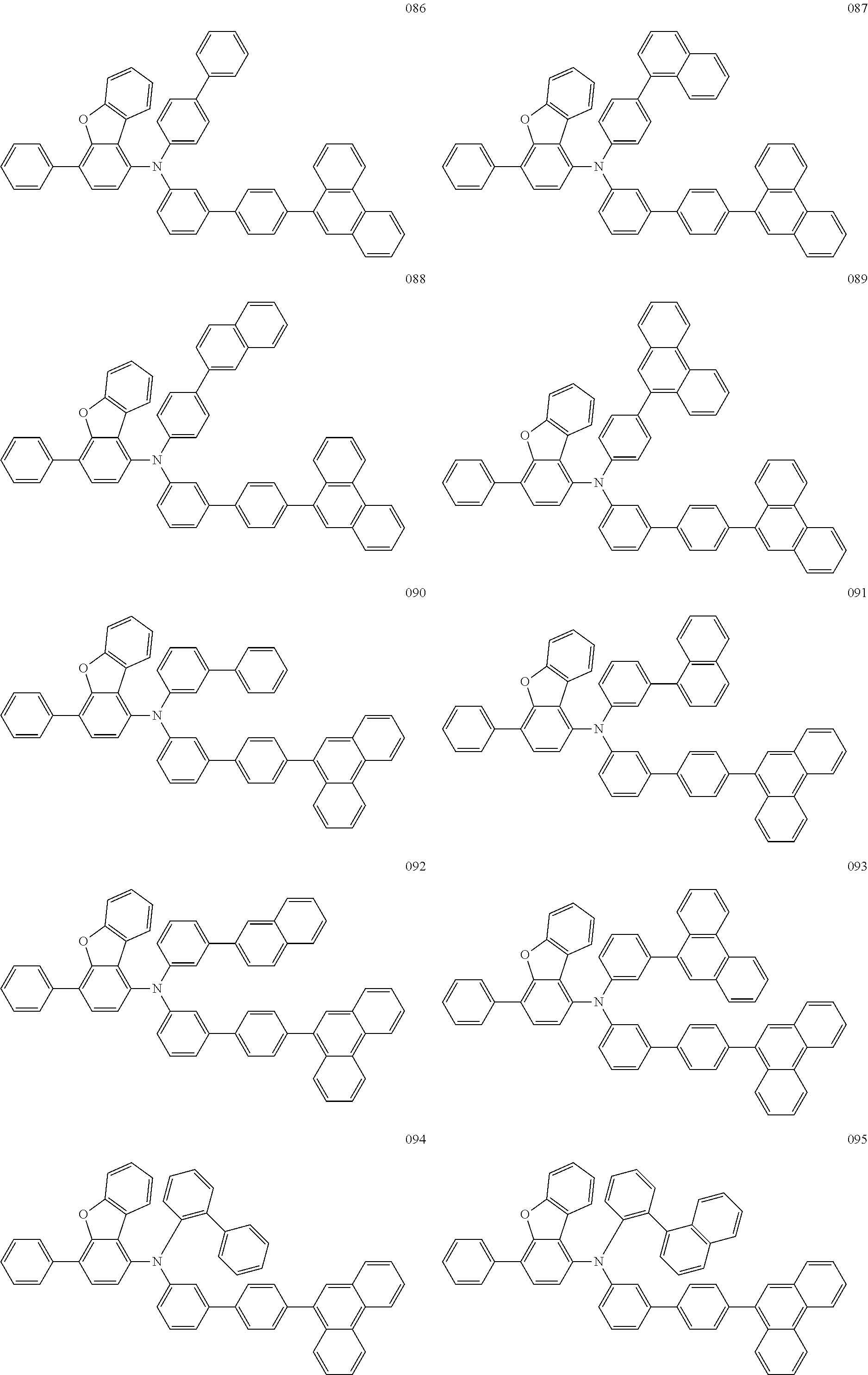

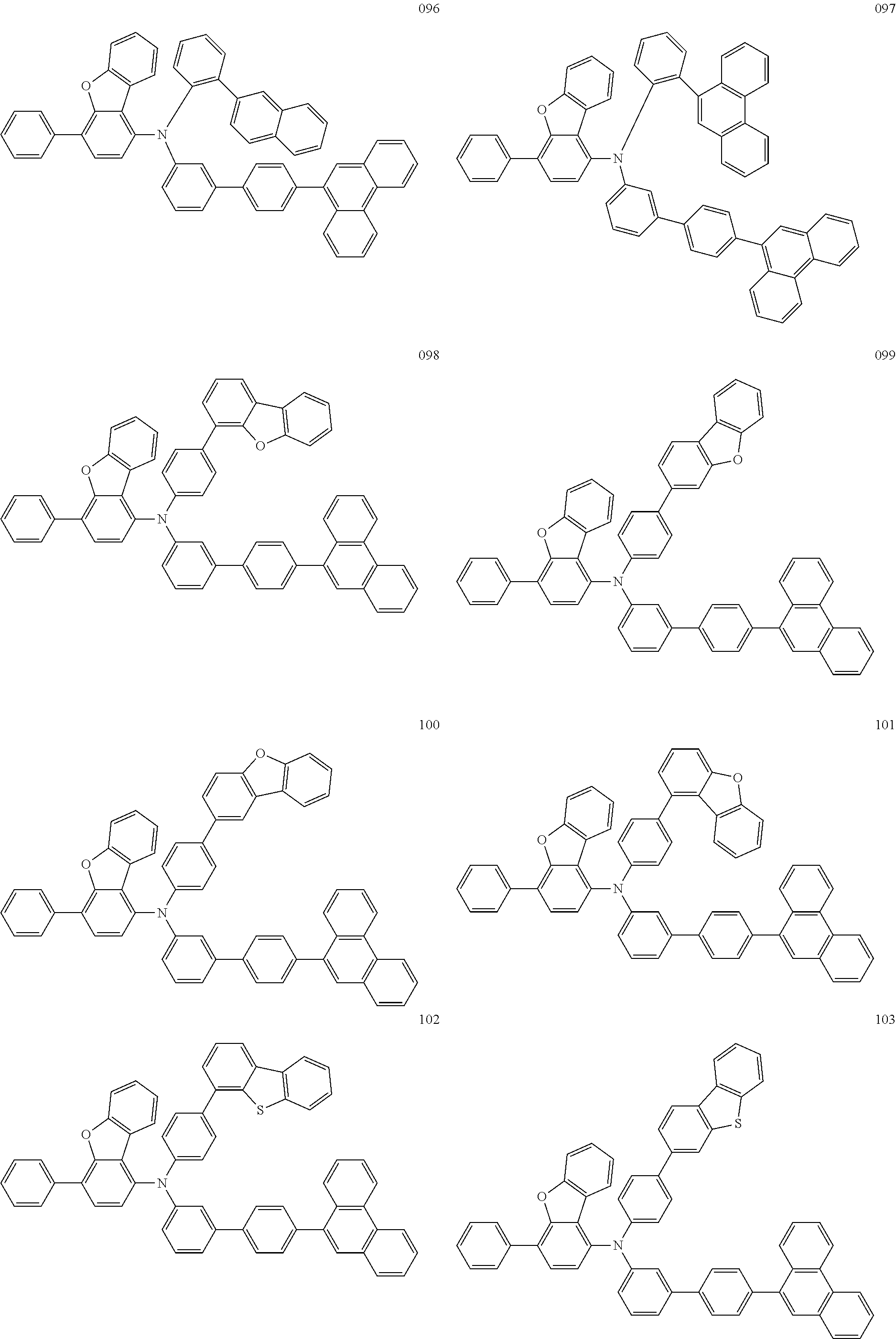

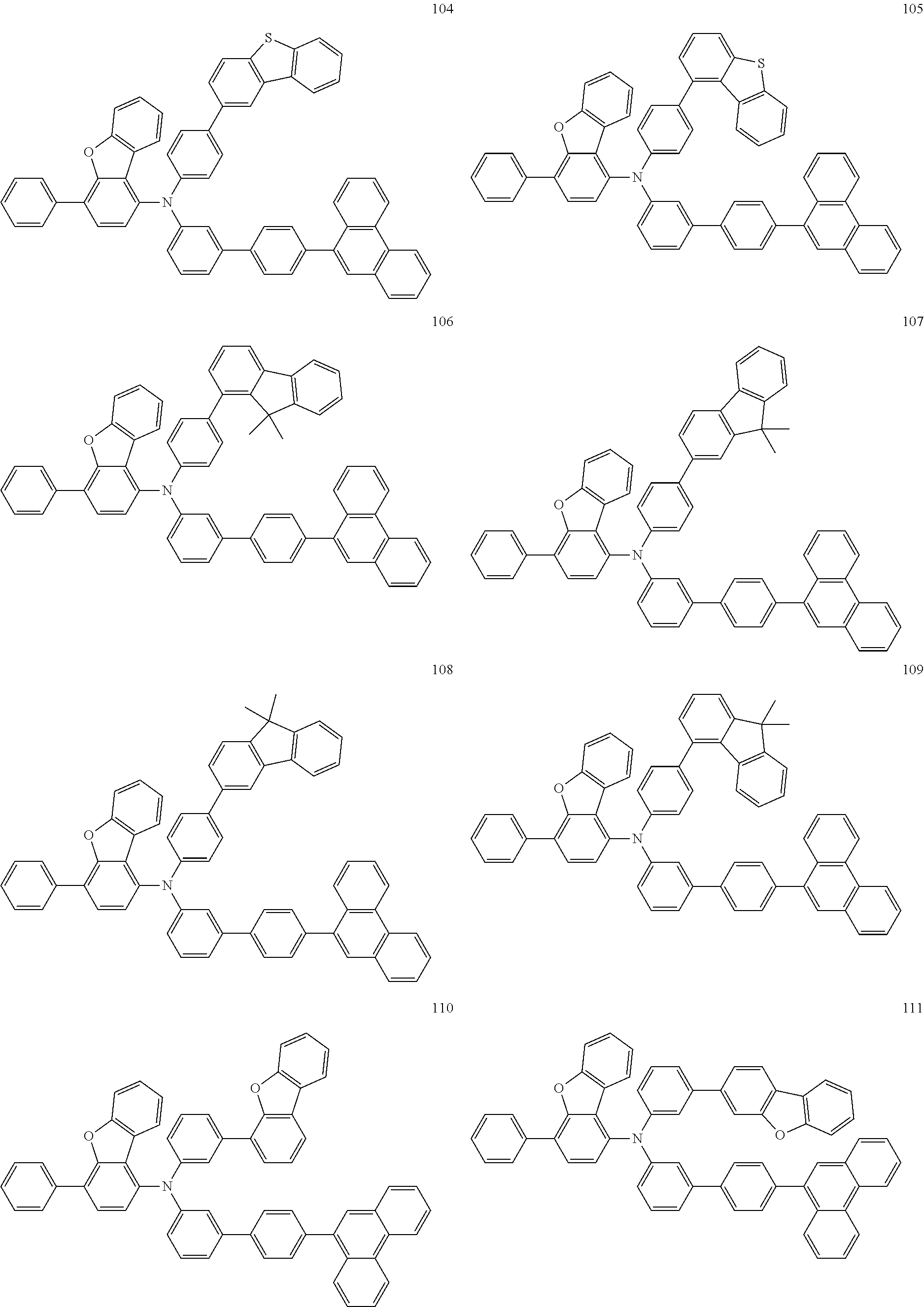

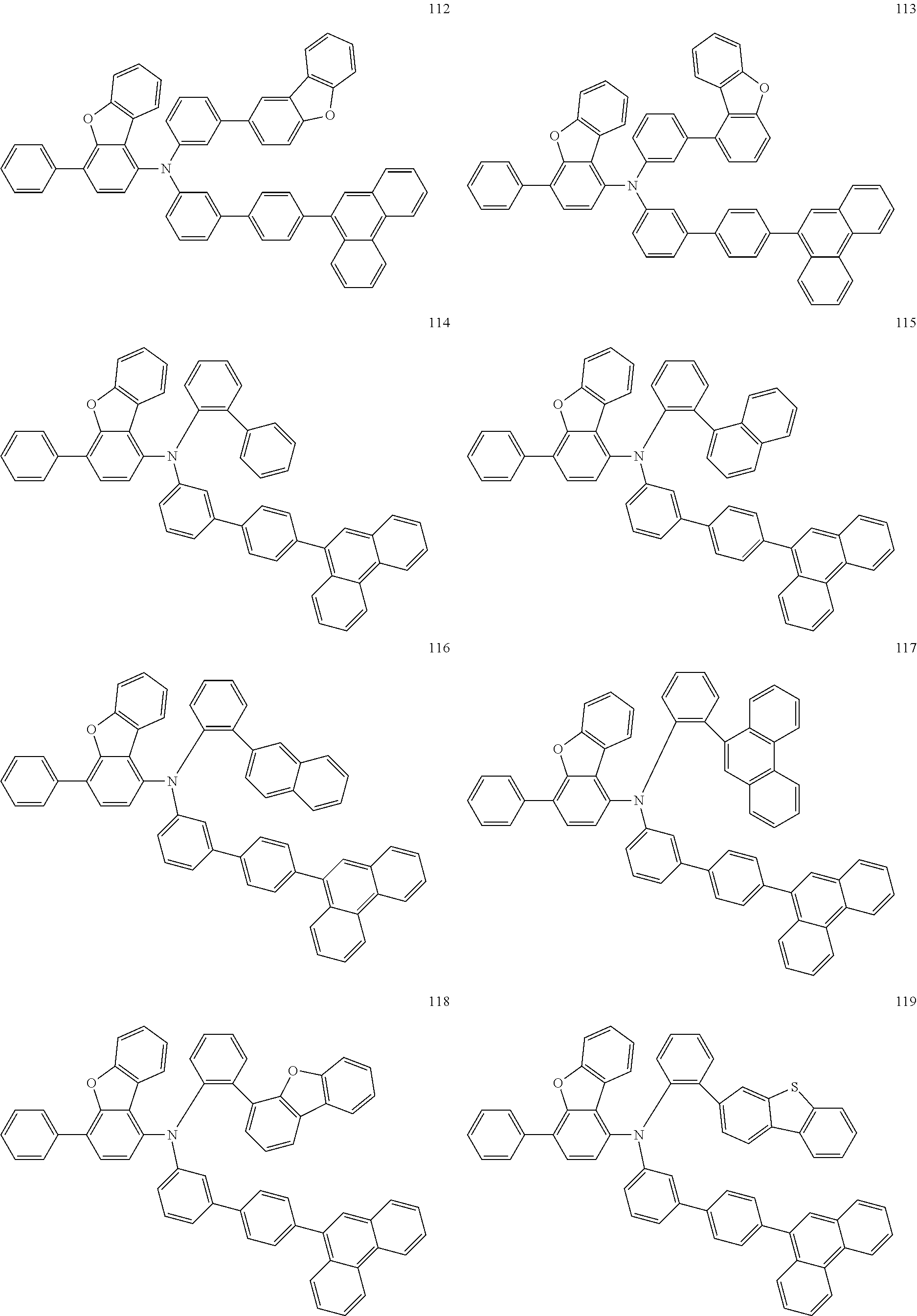

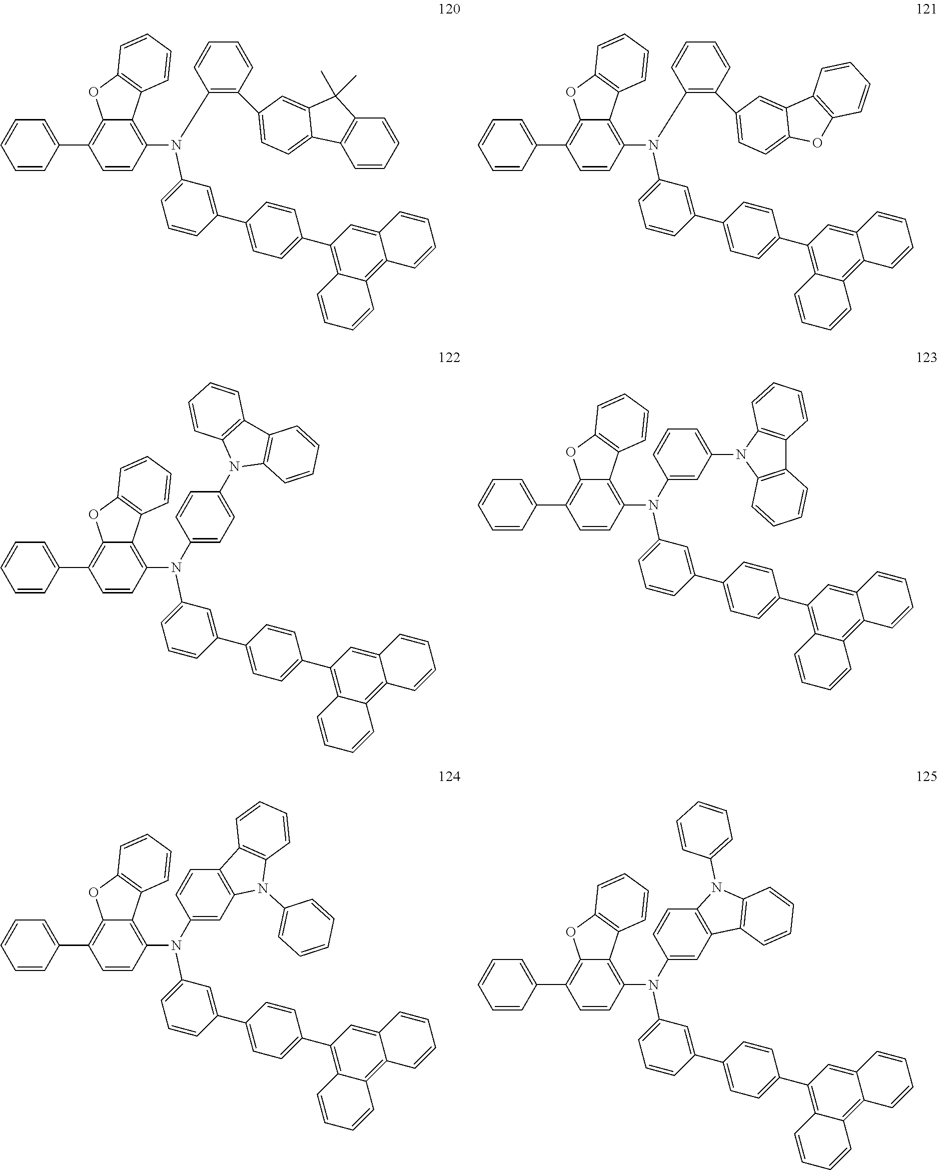

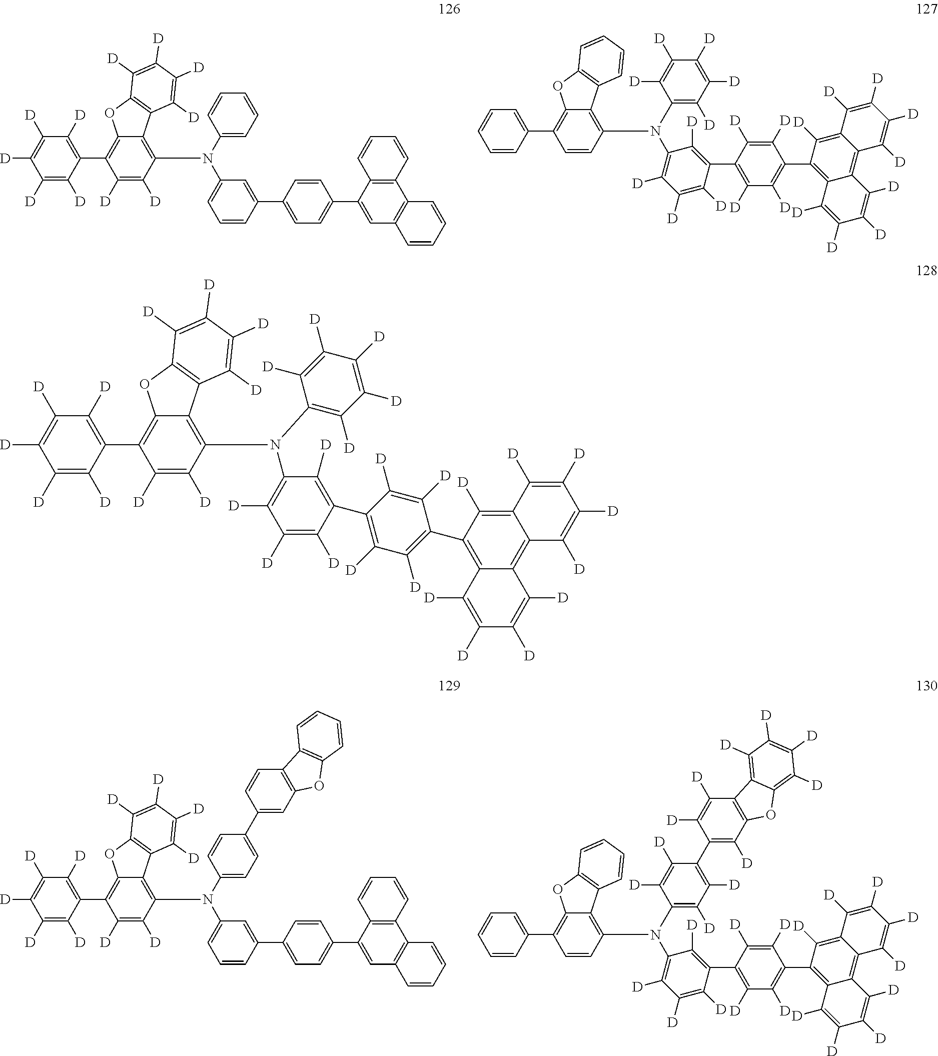

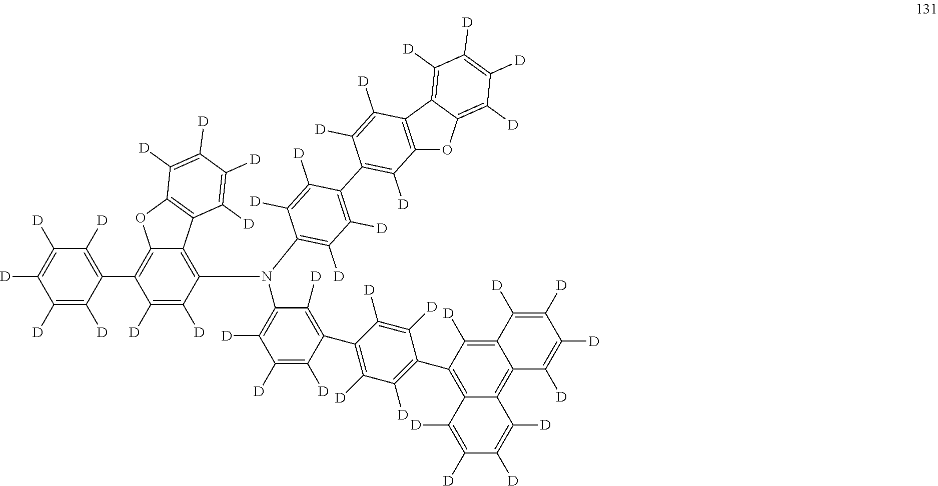 [SMILES: [2H]c1c([2H])c([2H])c(-c2c([2H])c([2H])c(N(c3c([2H])c([2H])c(-c4c([2H])c([2H])c5c(oc6c([2H])c([2H])c([2H])c([2H])c65)c4[2H])c([2H])c3[2H])c3c([2H])c([2H])c([2H])c(-c4c([2H])c([2H])c(-c5c([2H])c6c([2H])c([2H])c([2H])c([2H])c6c6c([2H])c([2H])c([2H])c([2H])c56)c([2H])c4[2H])c3[2H])c3c2oc2c([2H])c([2H])c([2H])c([2H])c23)c([2H])c1[2H]]

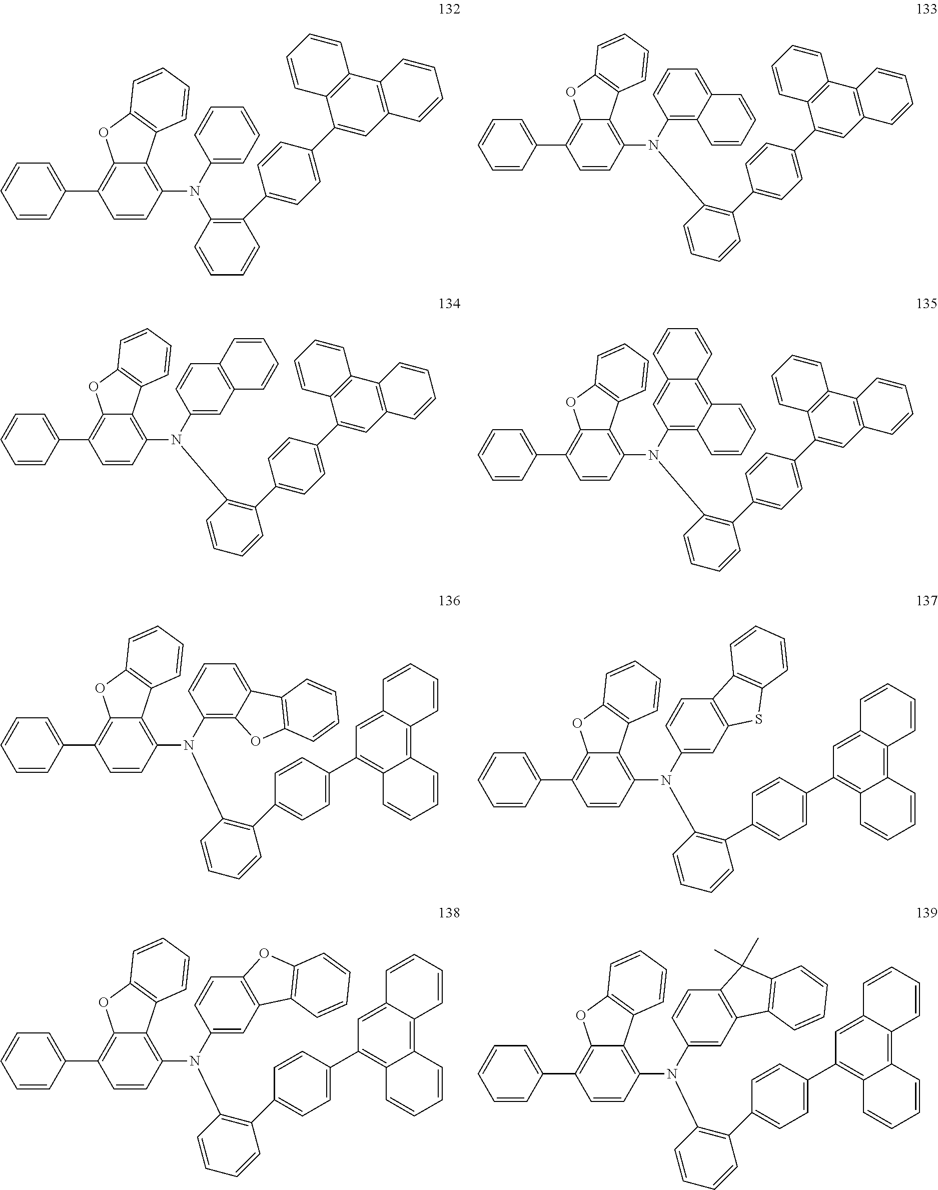

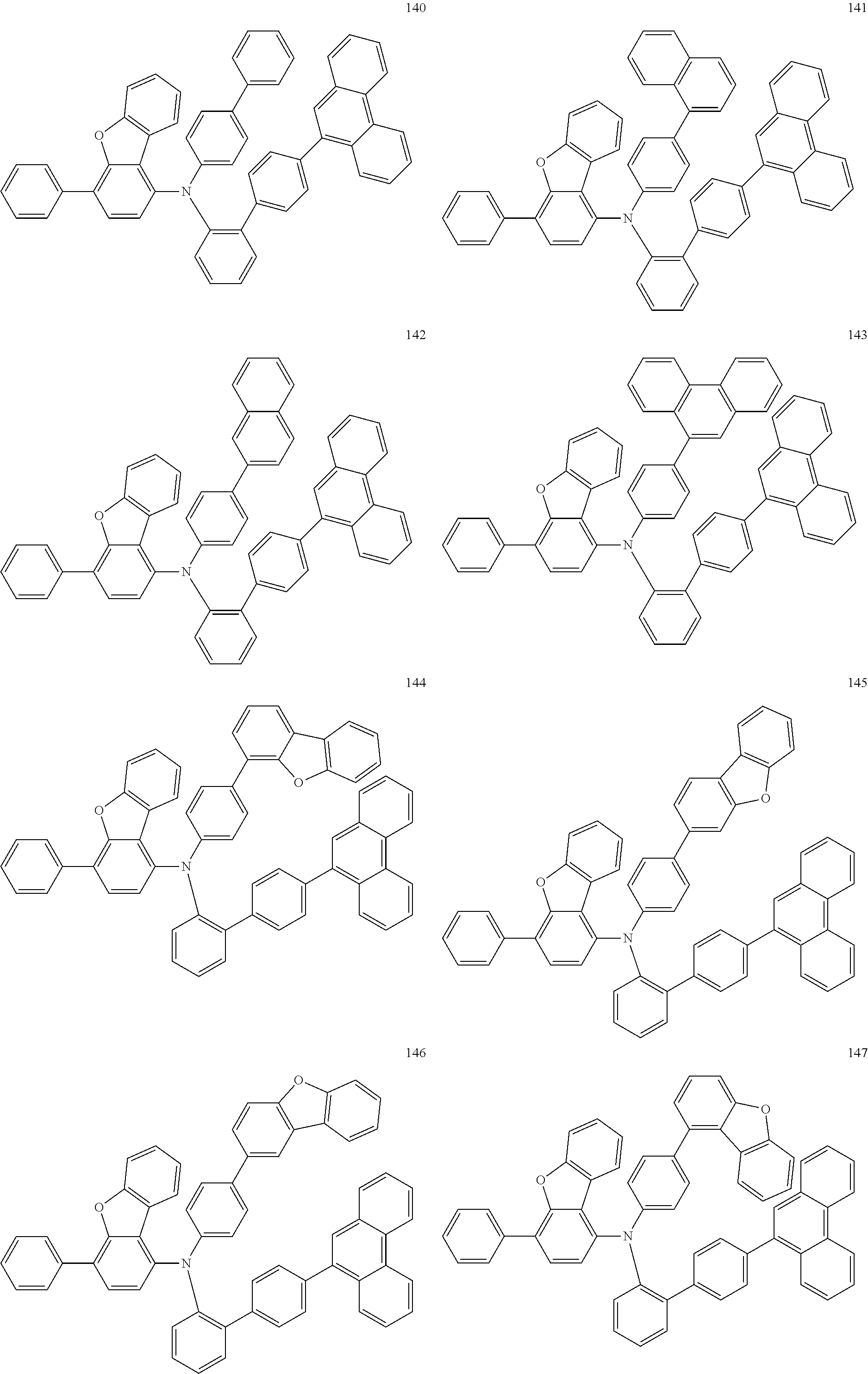

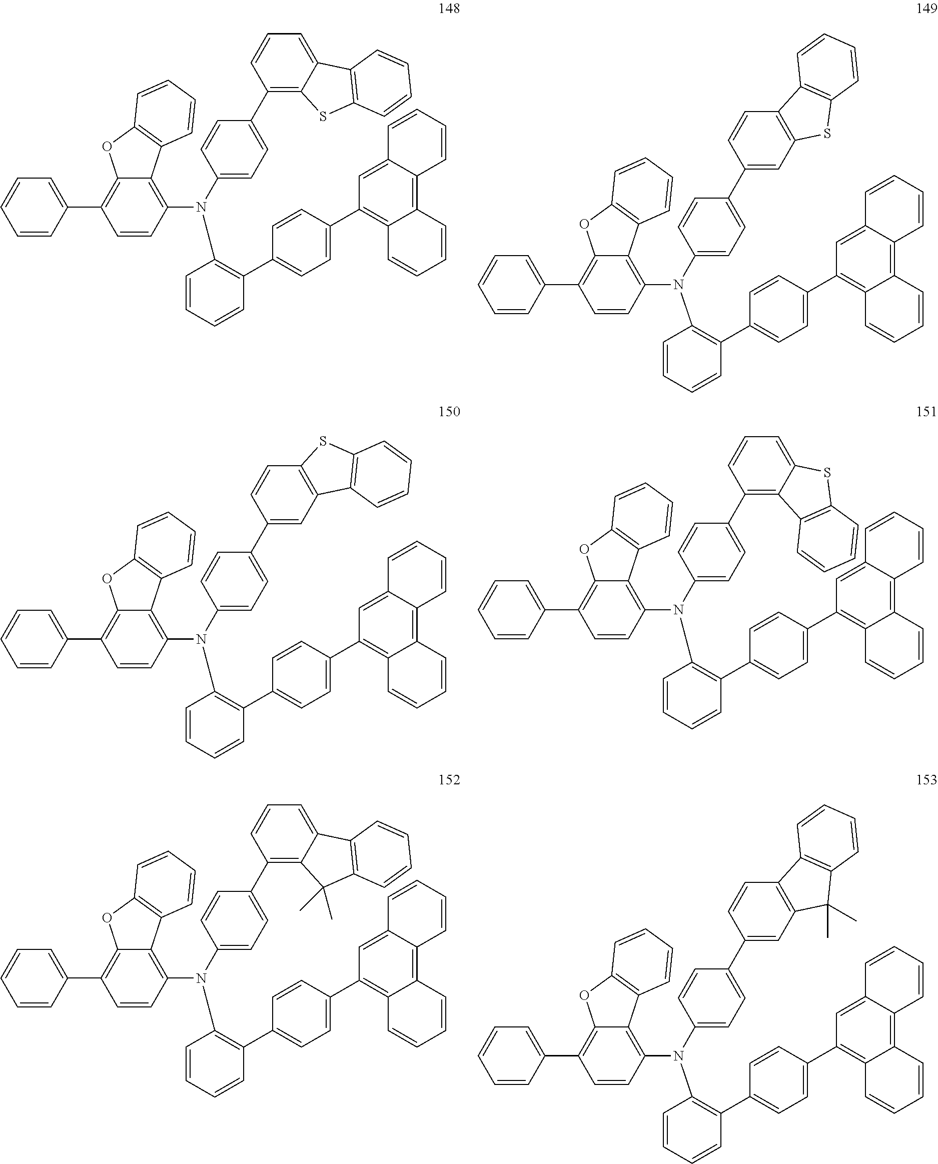

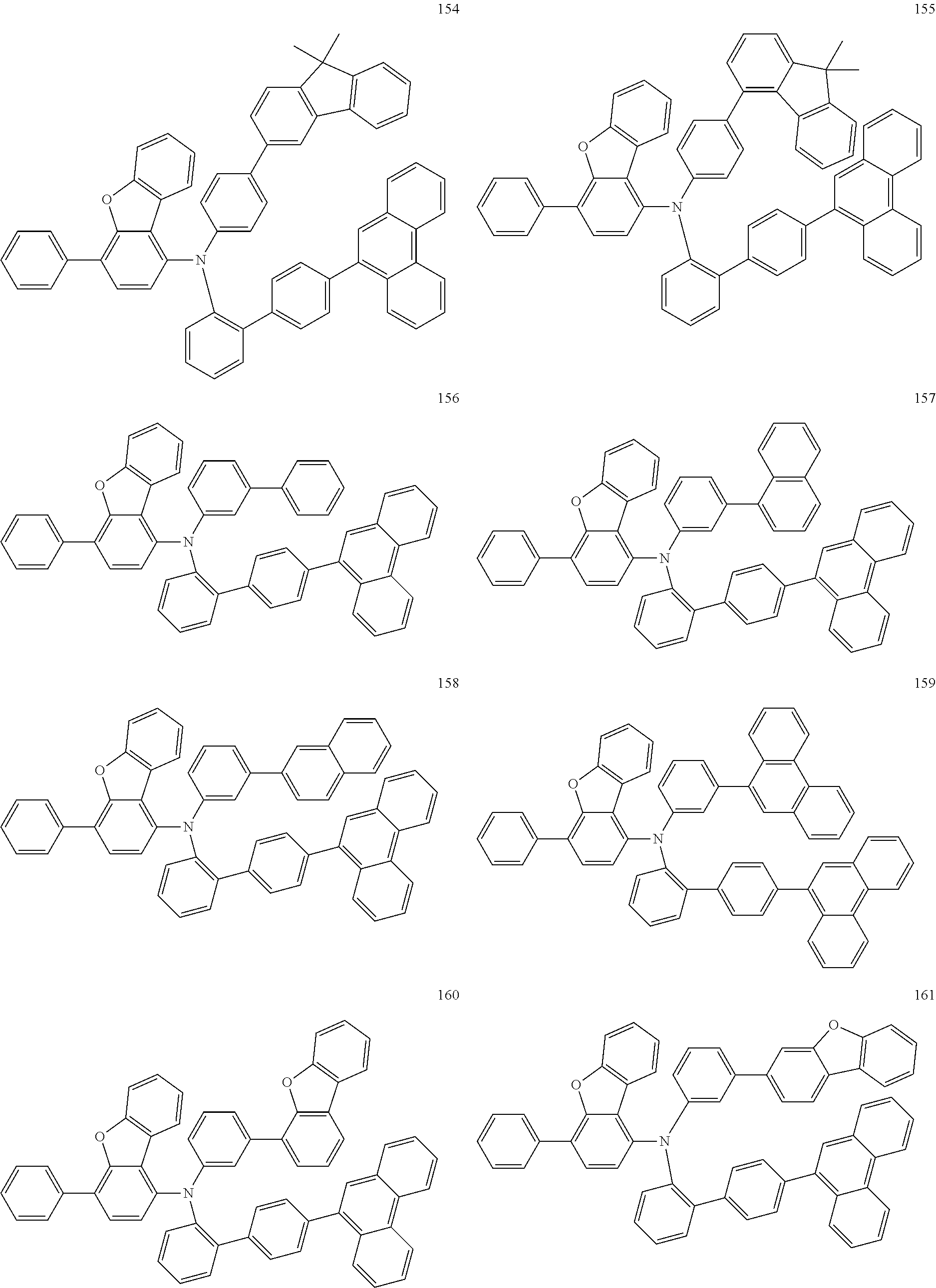

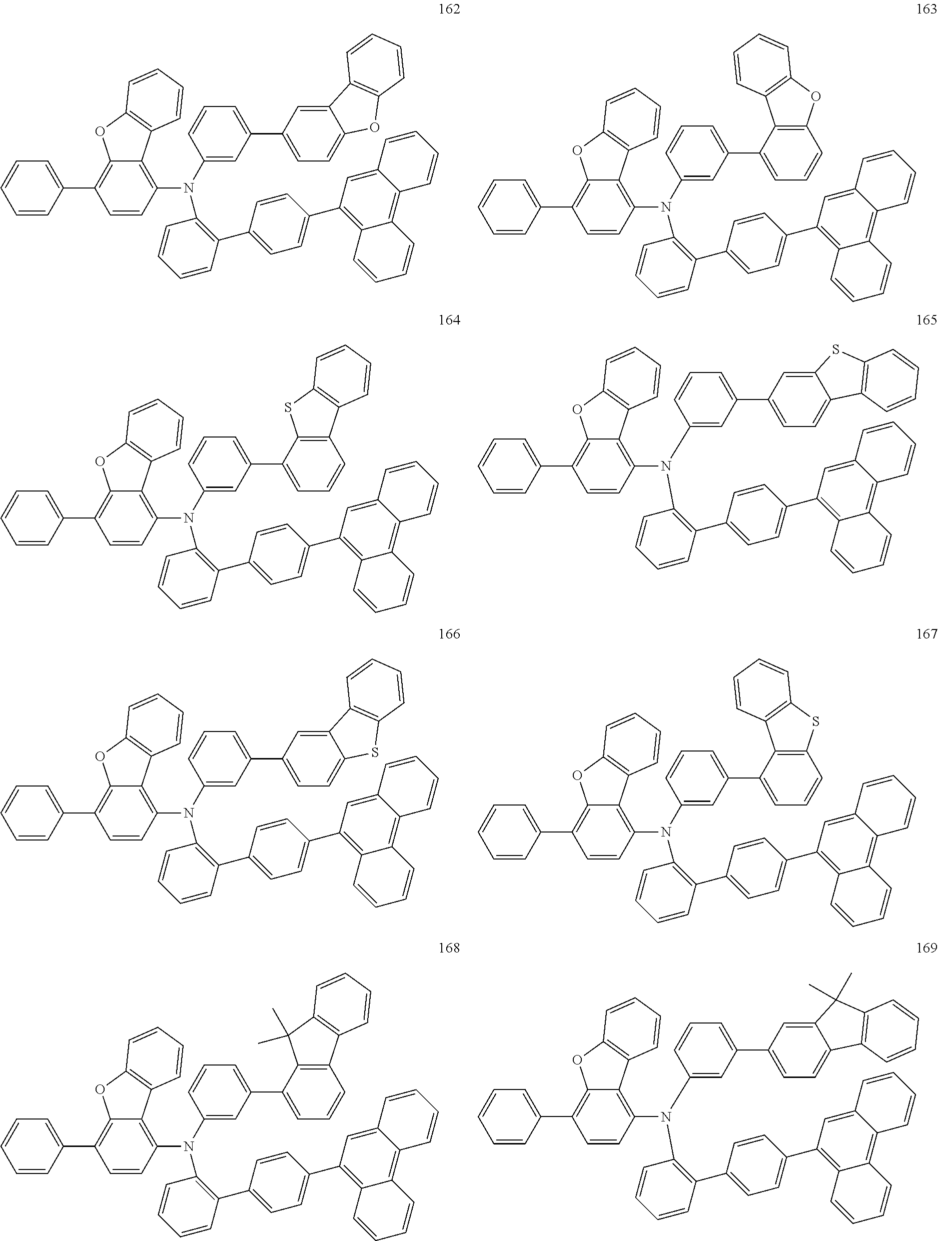

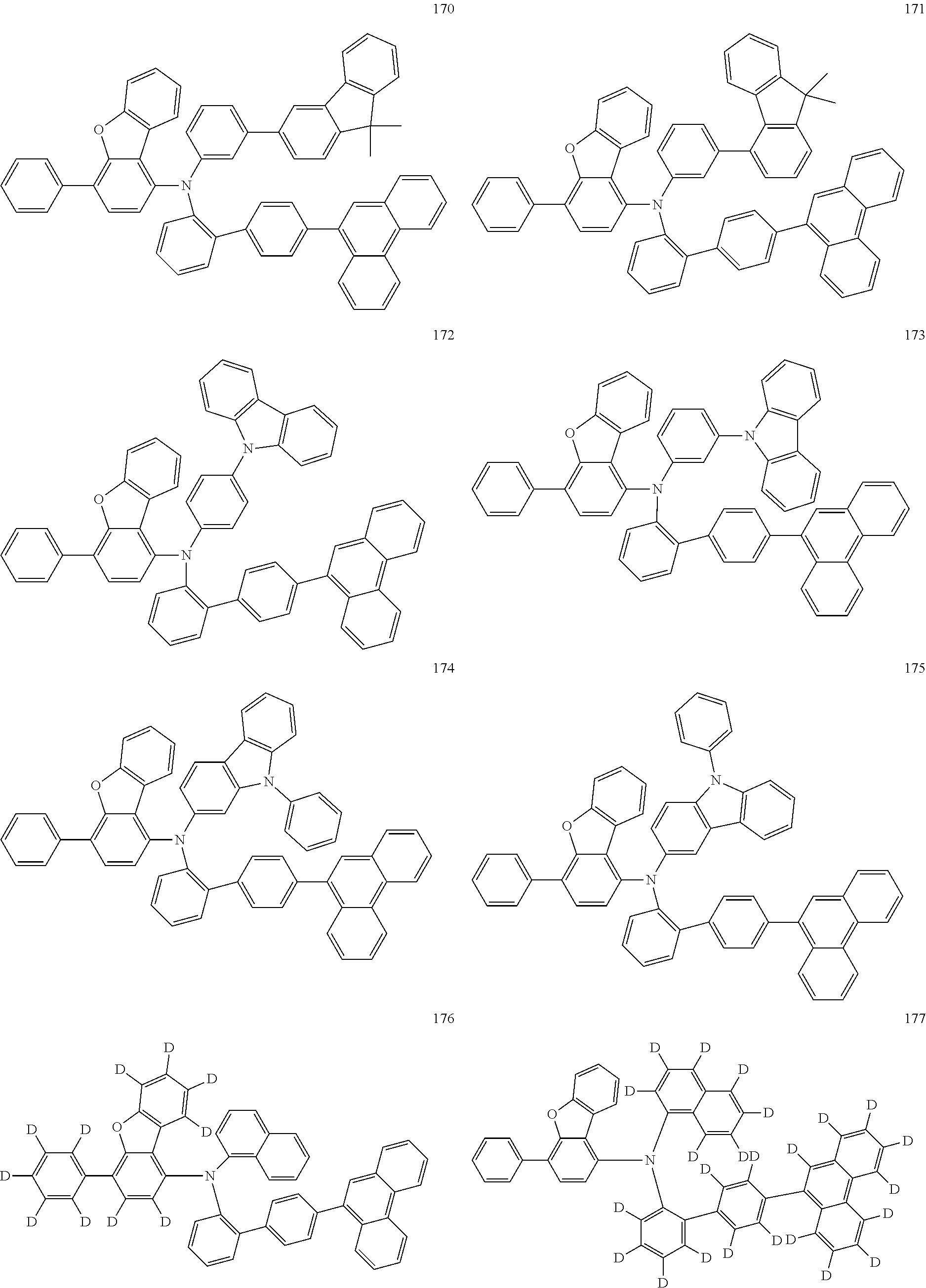

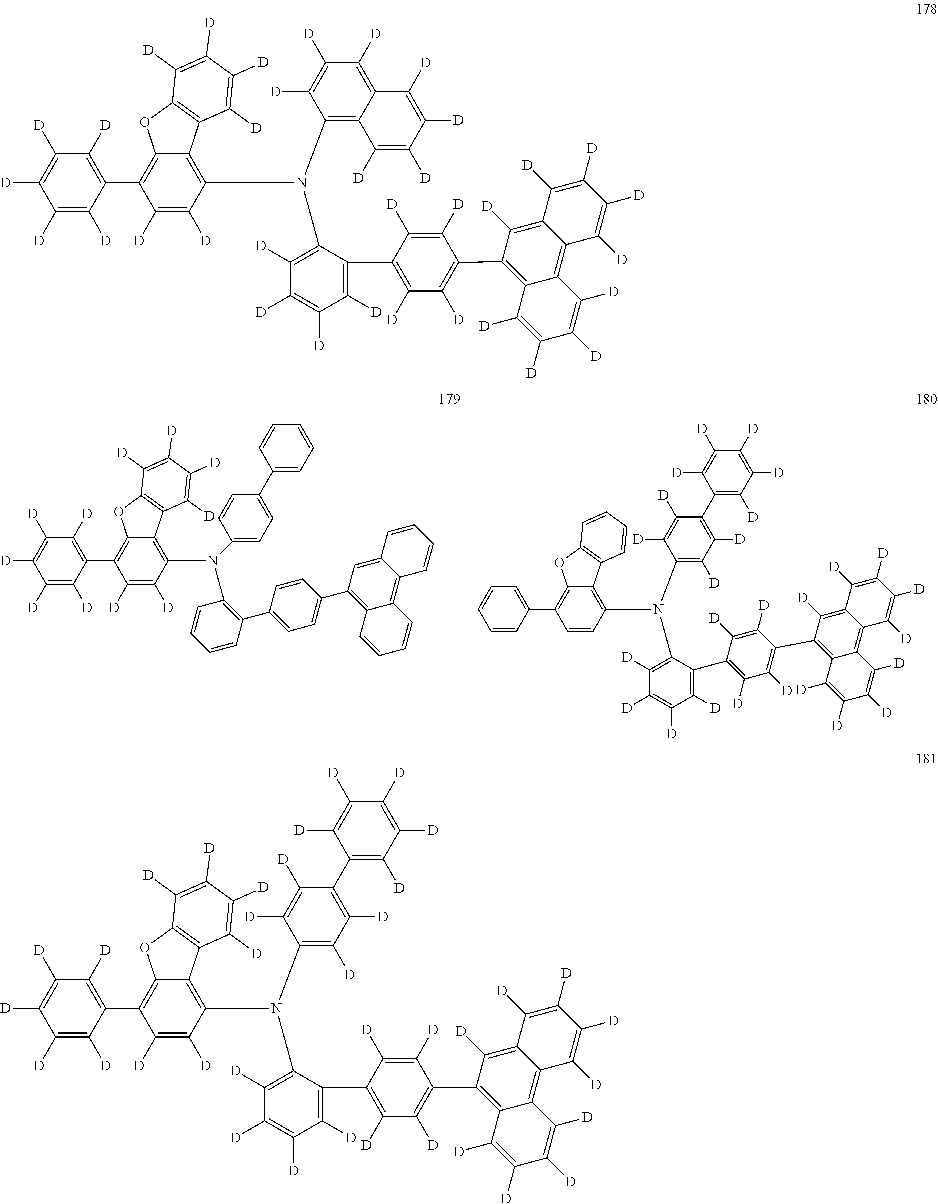

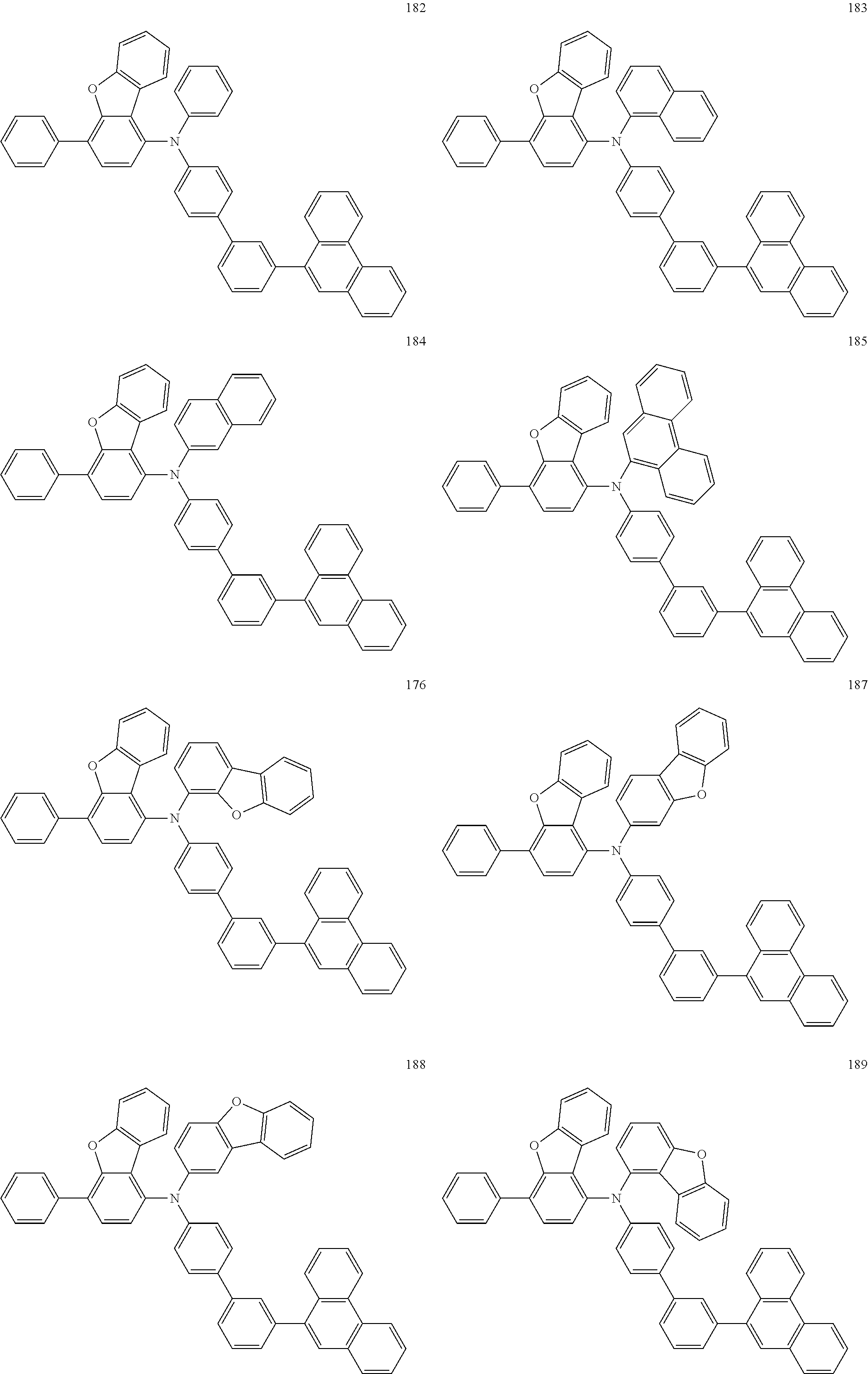

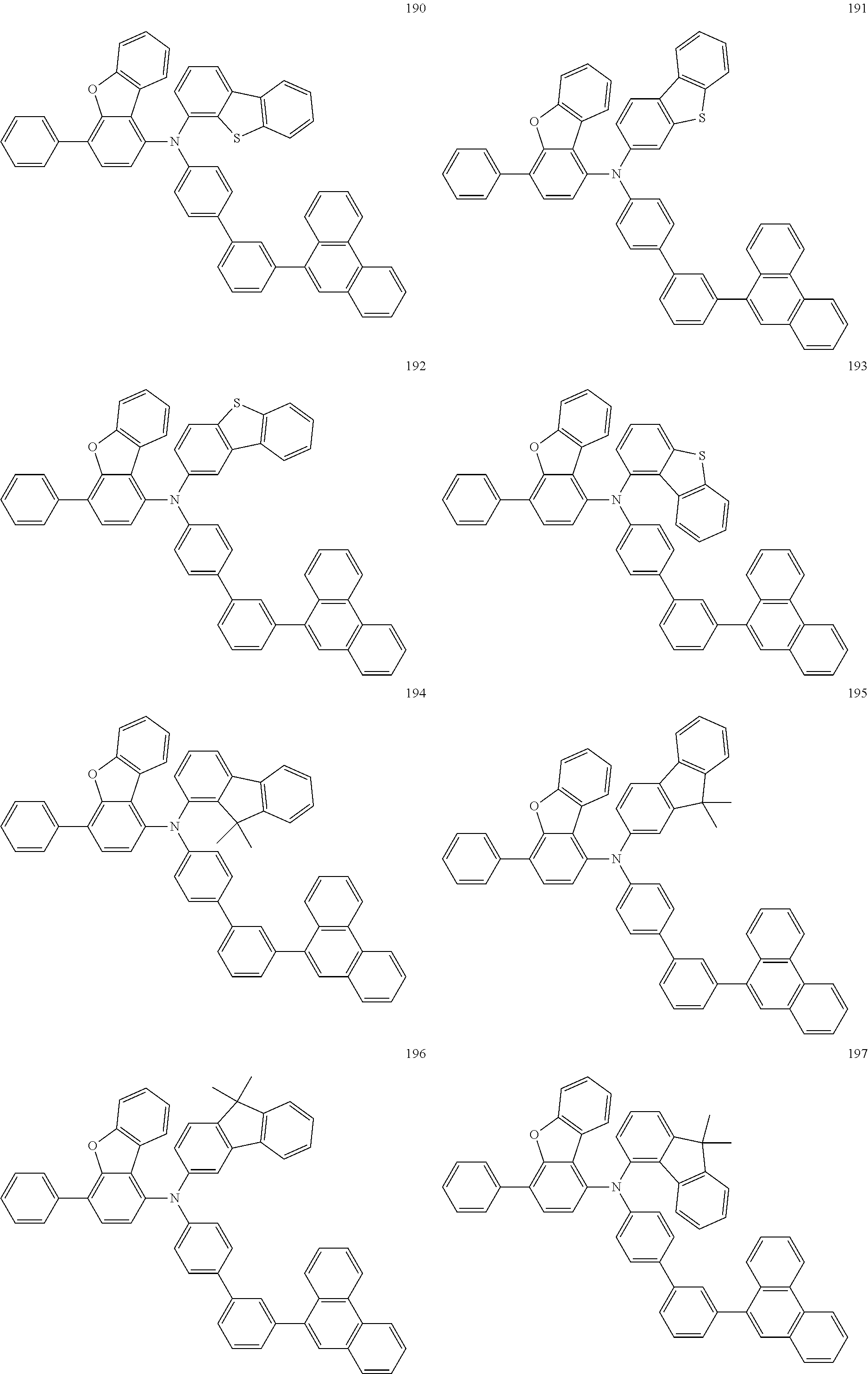

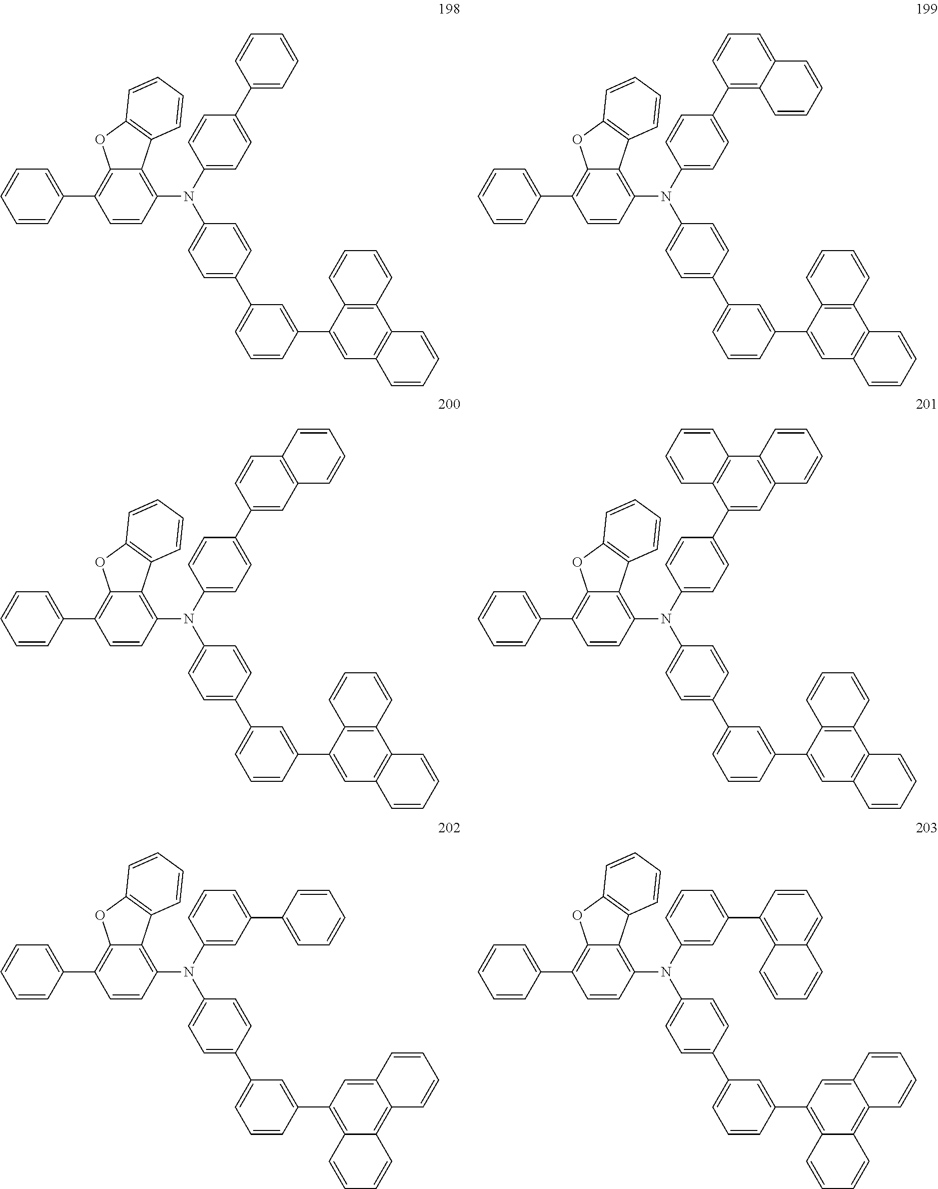

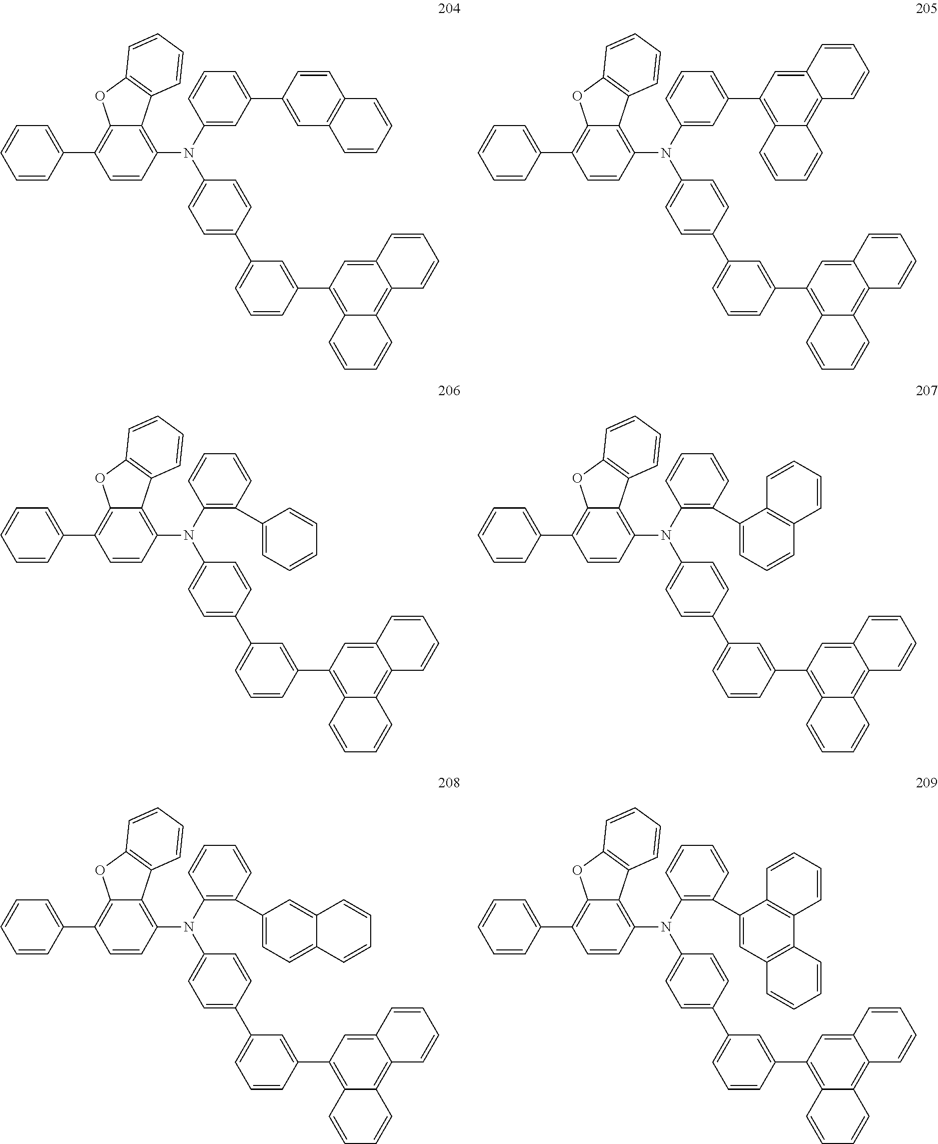

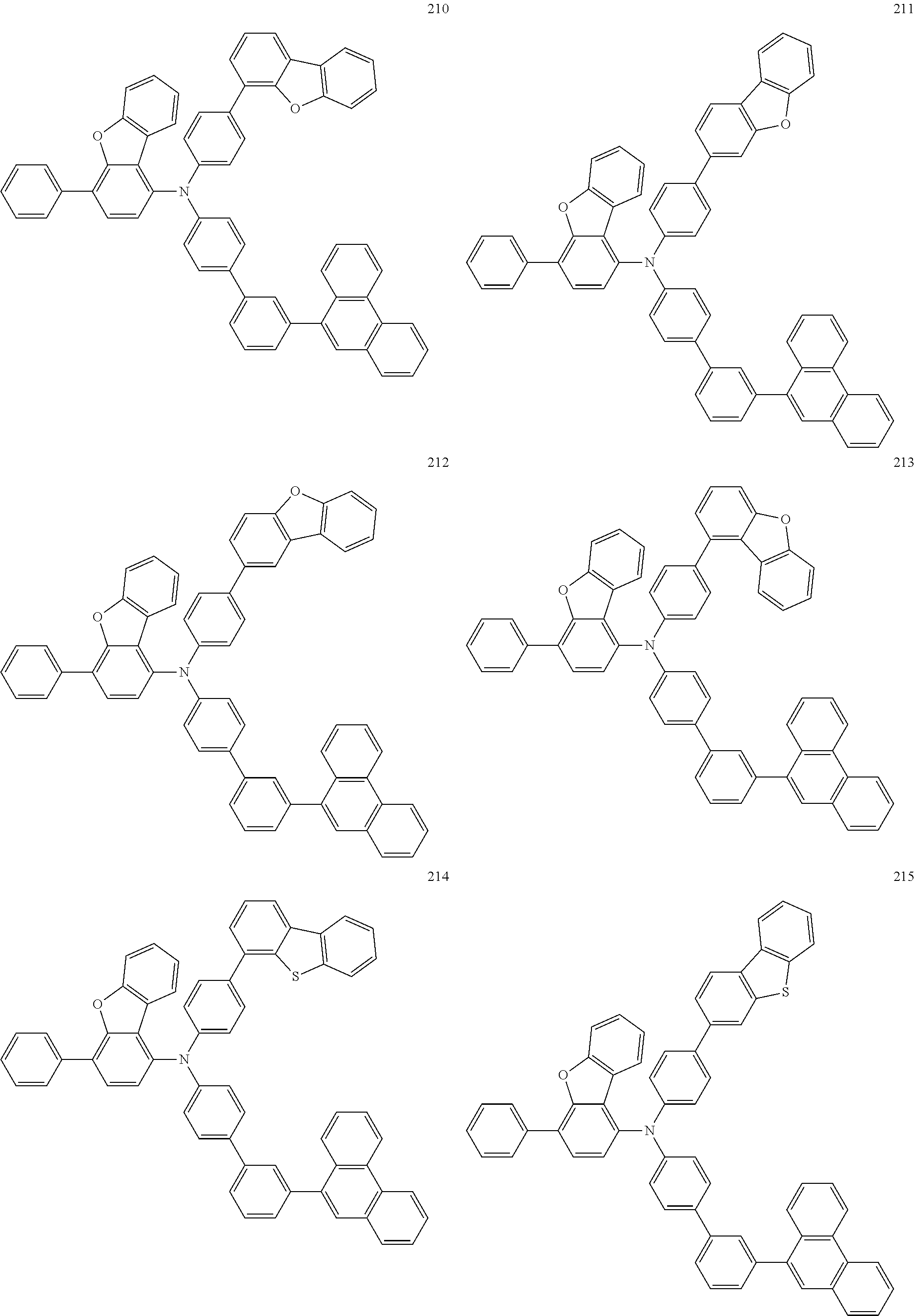

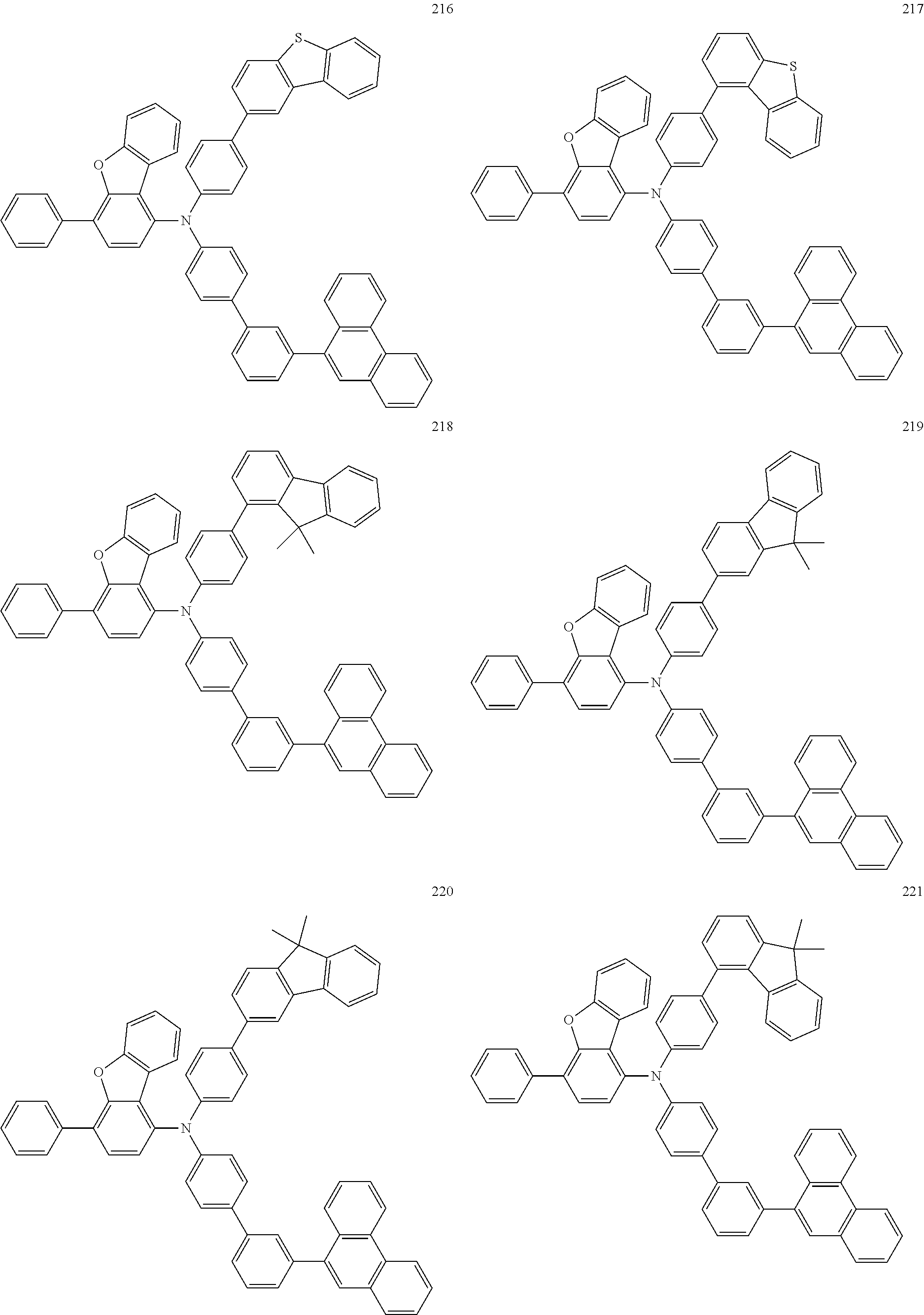

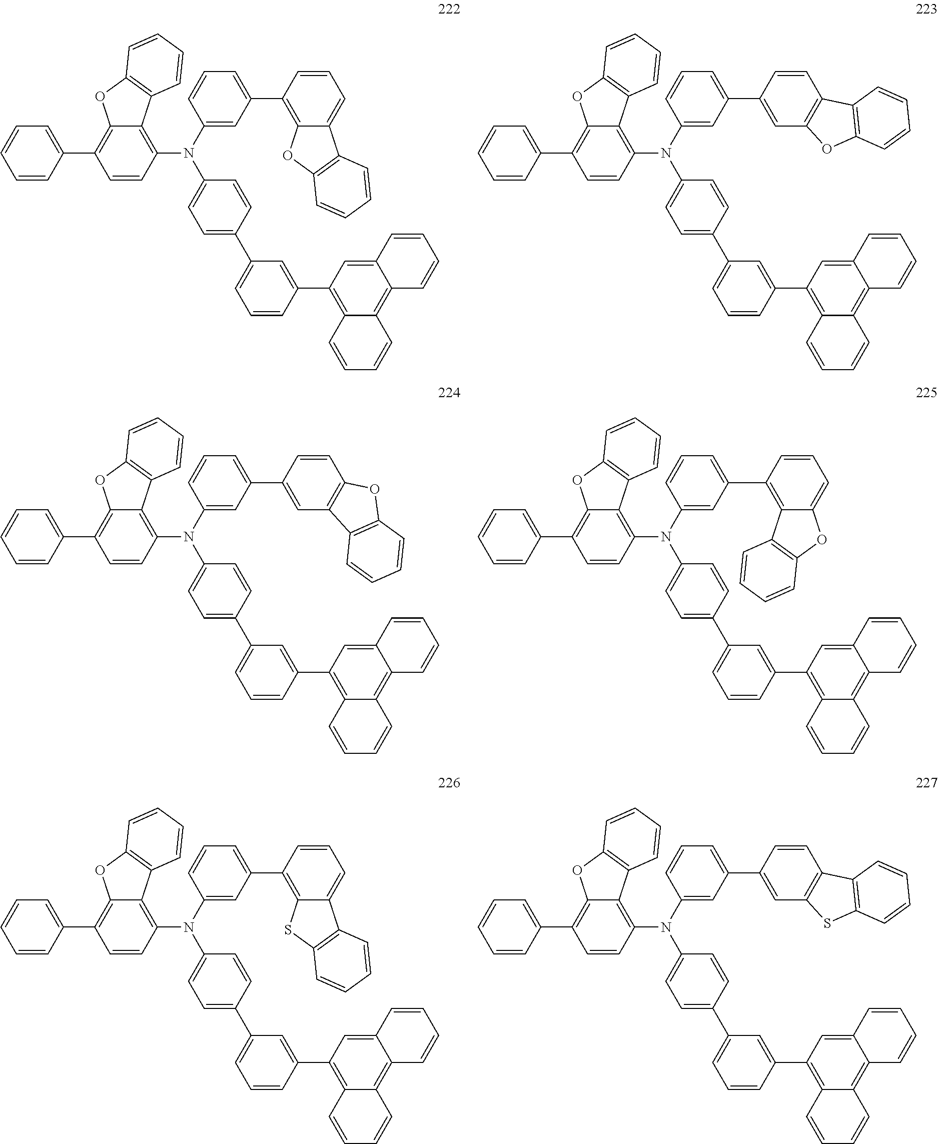

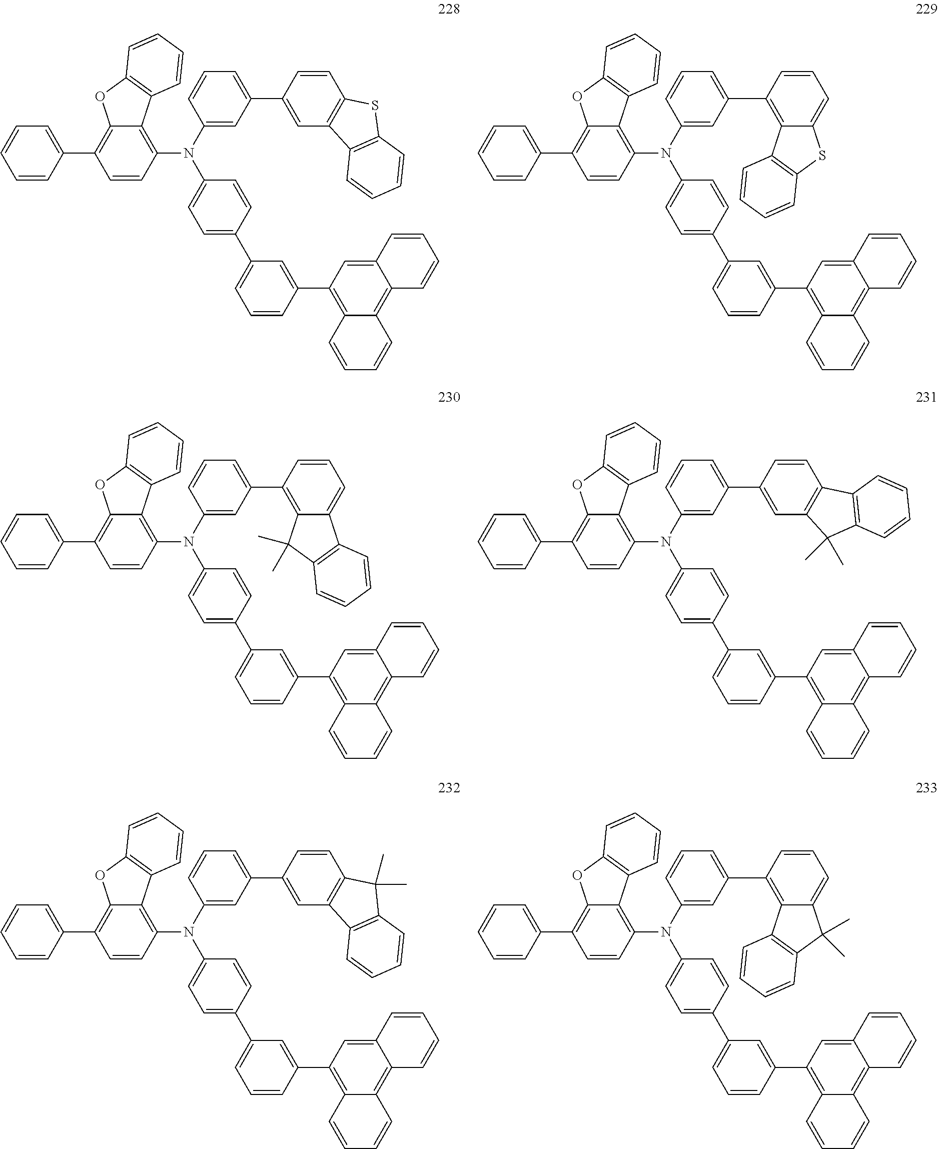

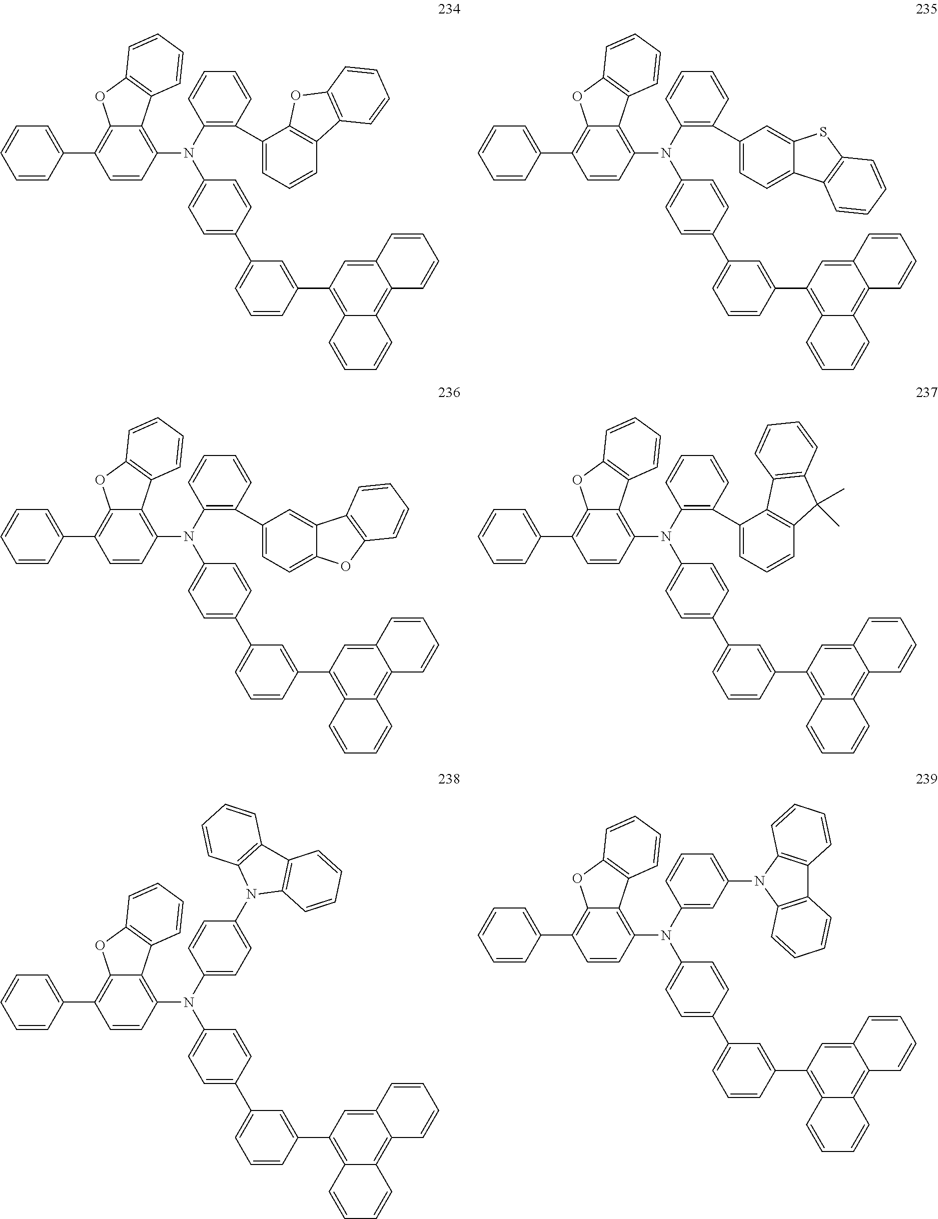

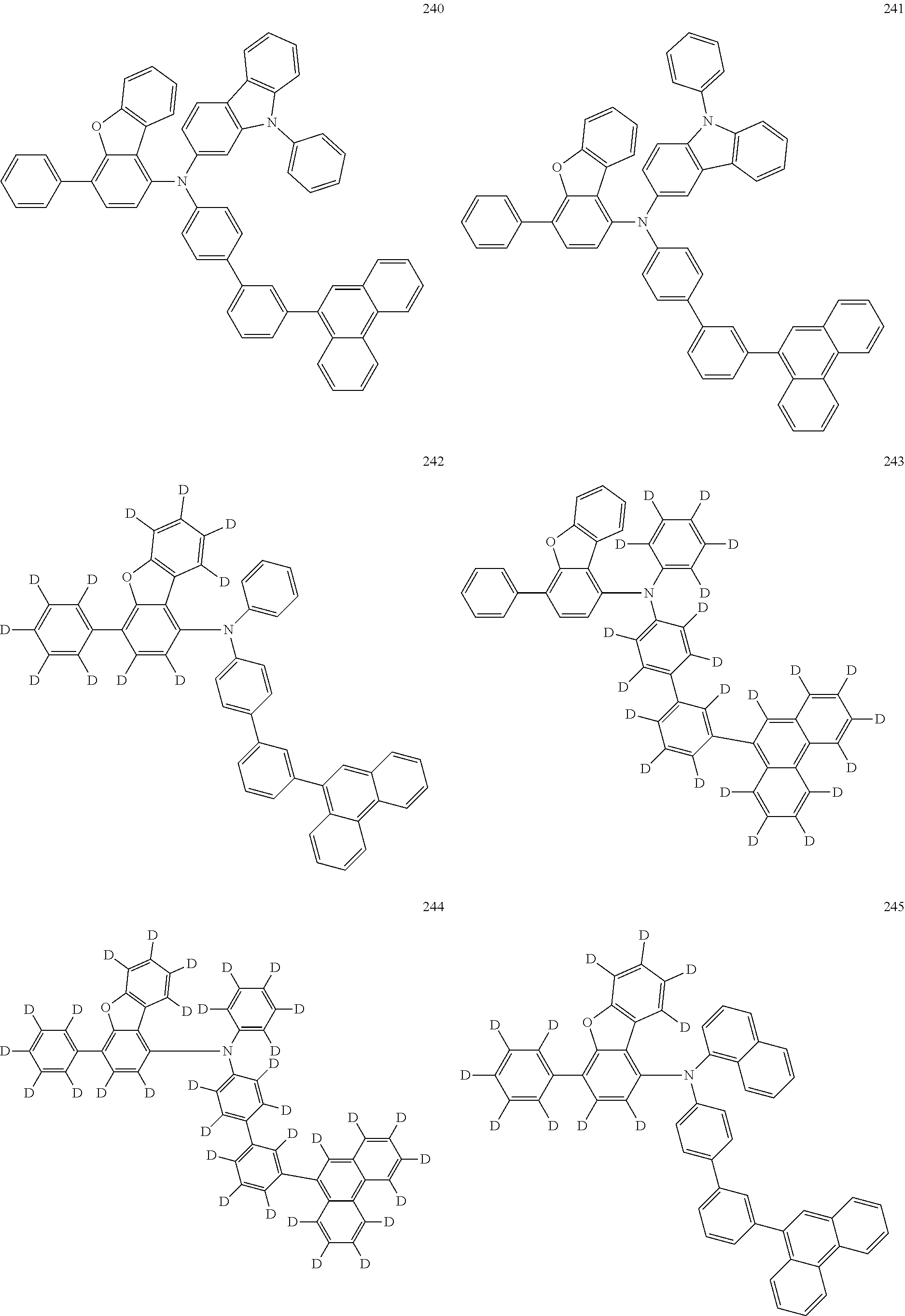

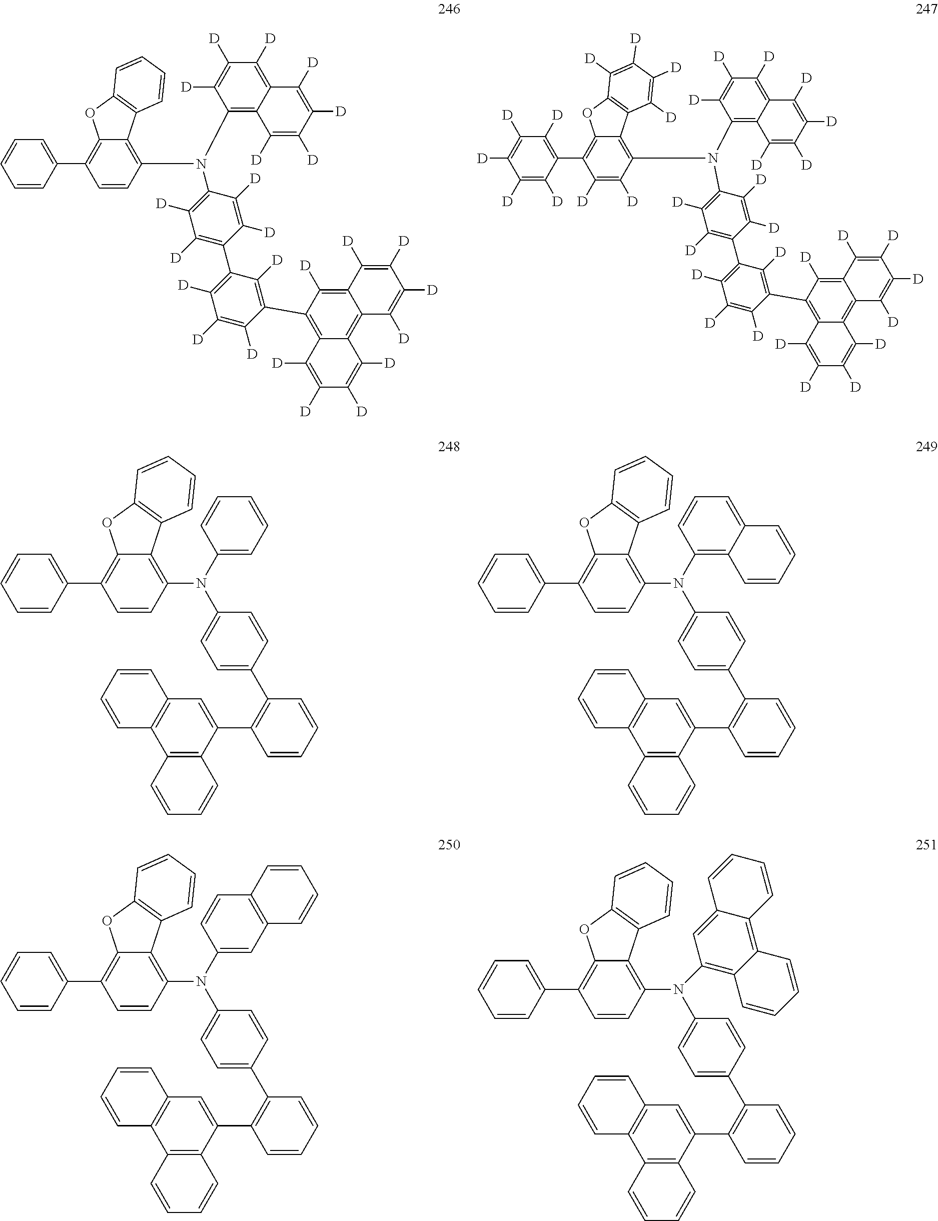

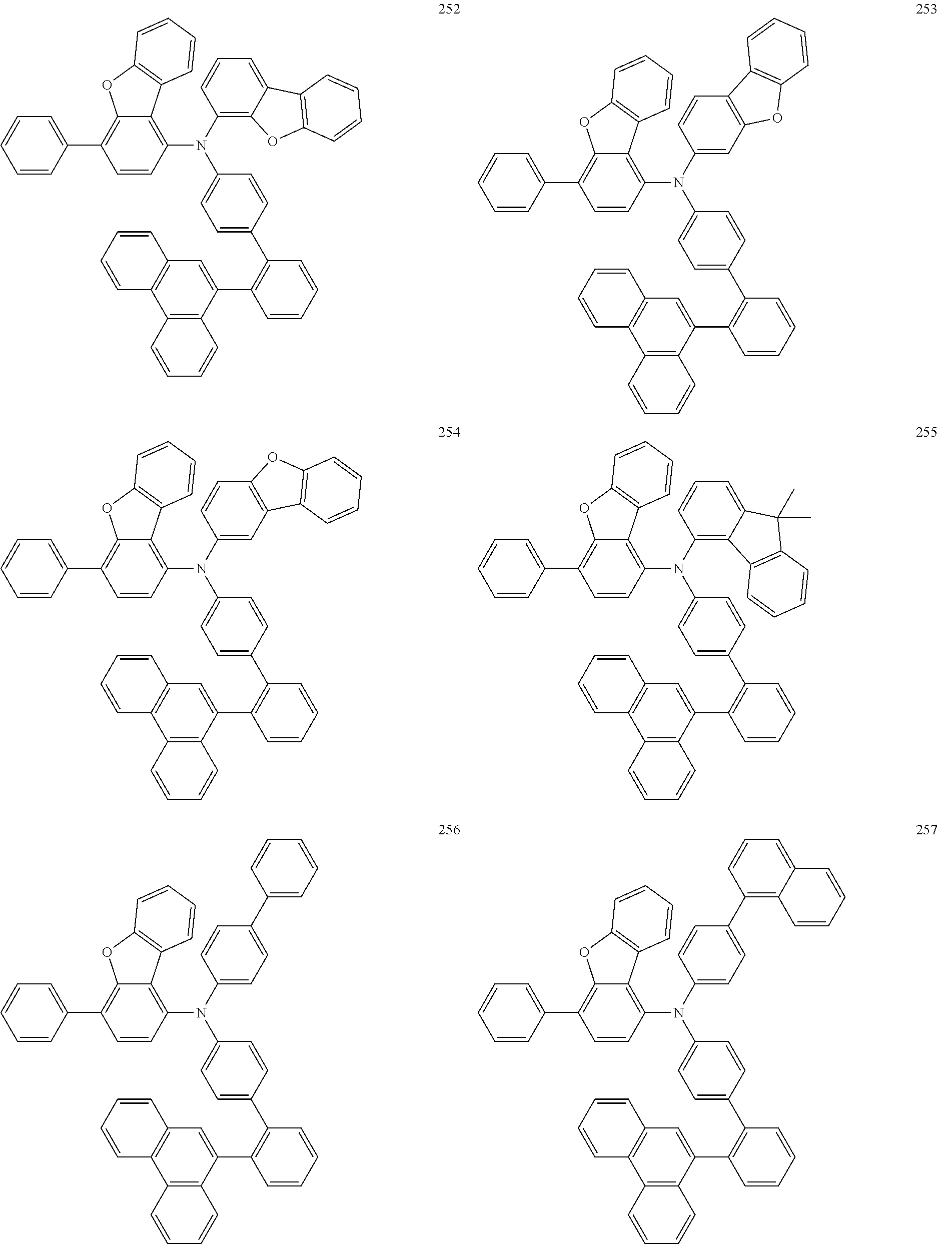

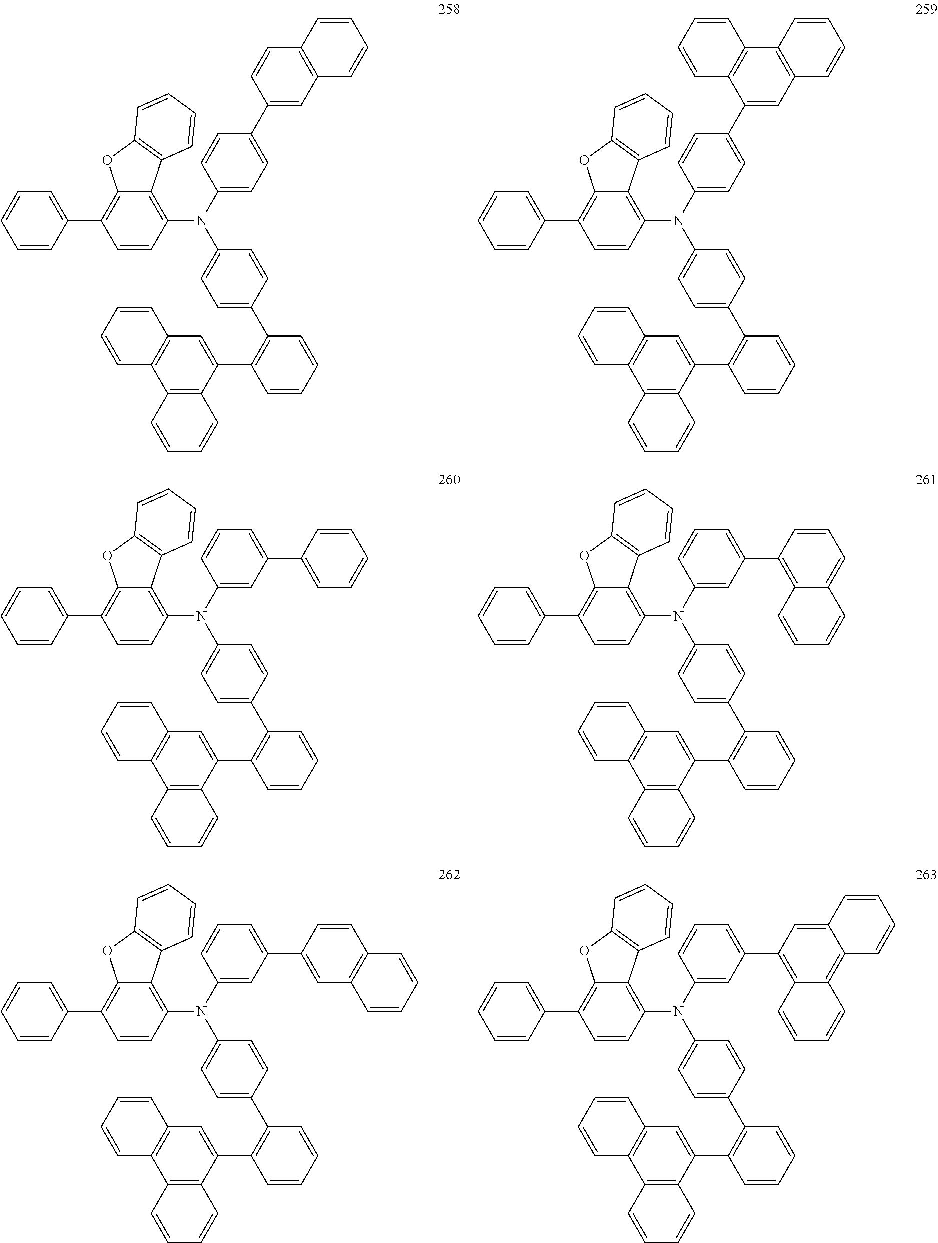

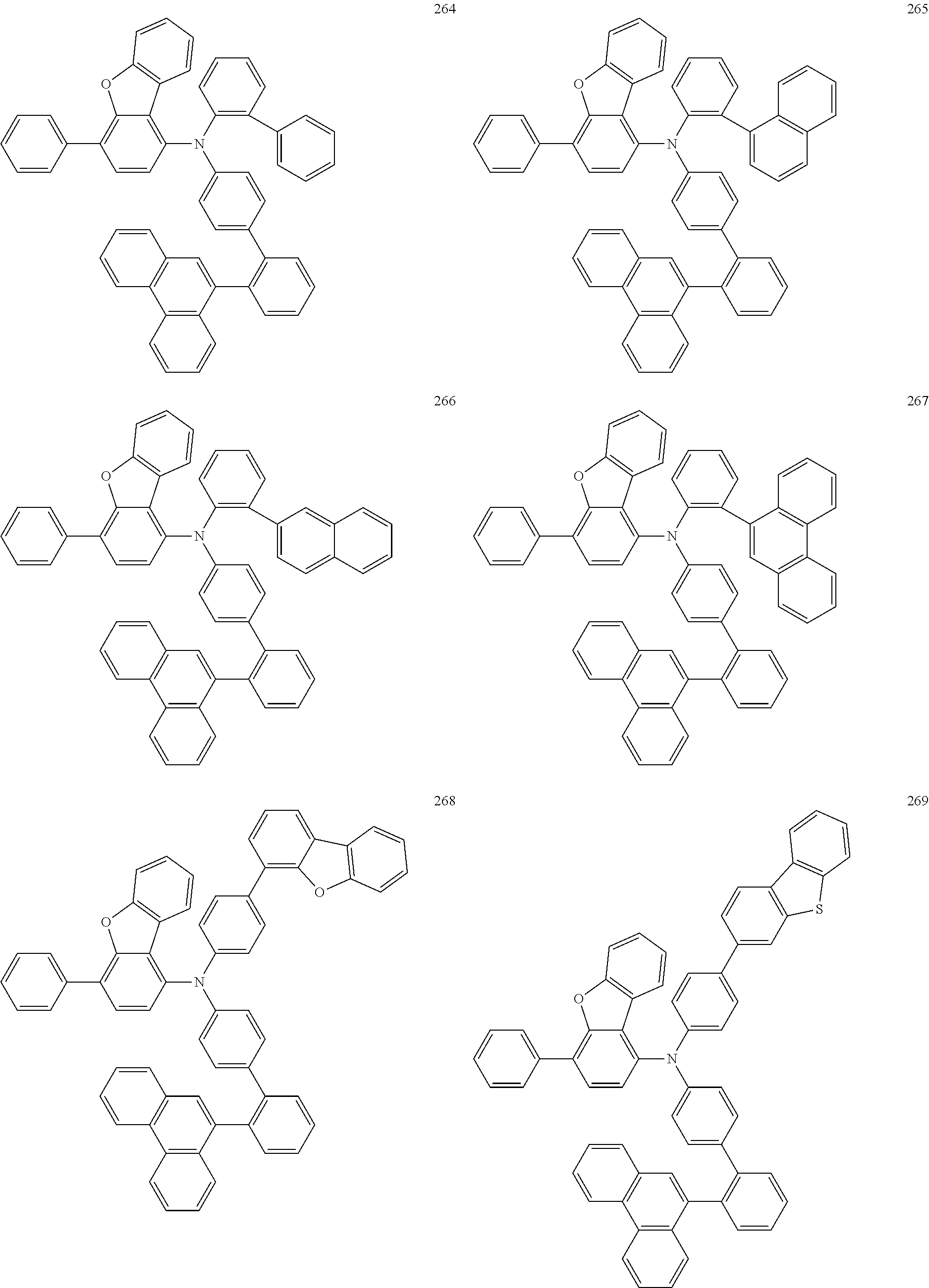

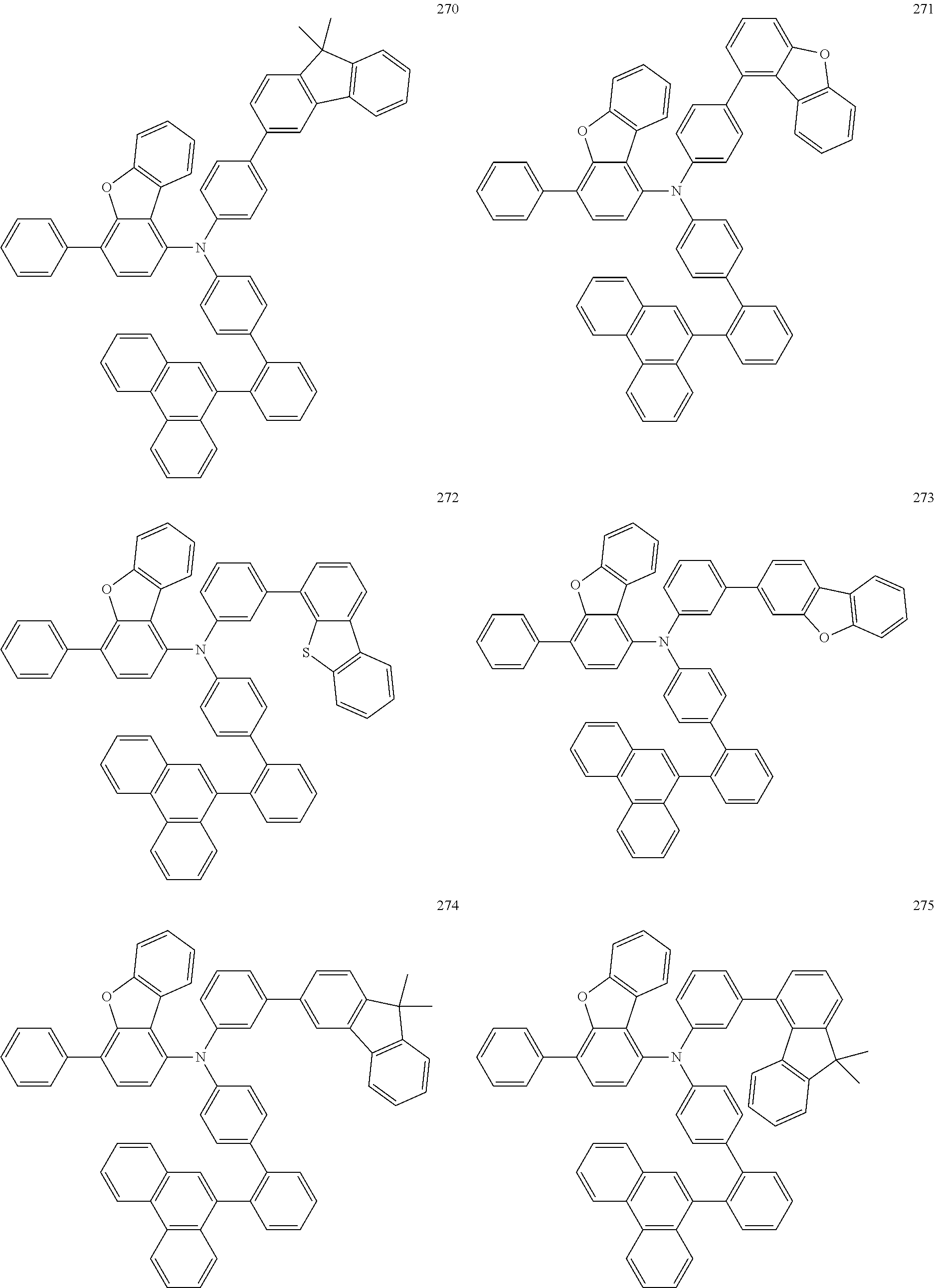

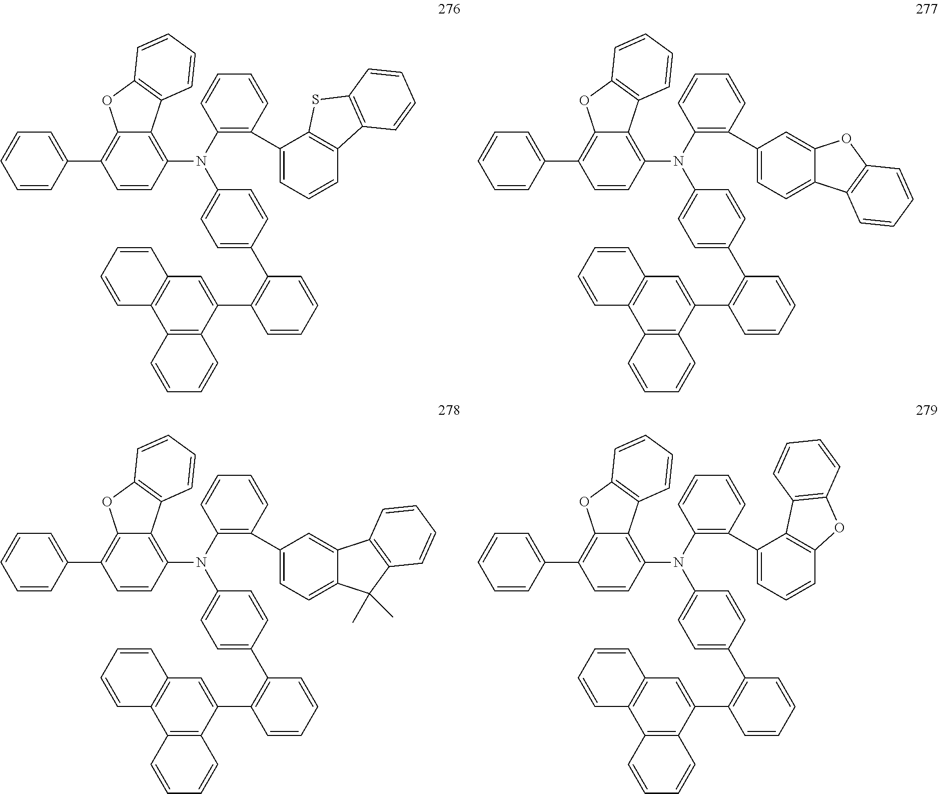

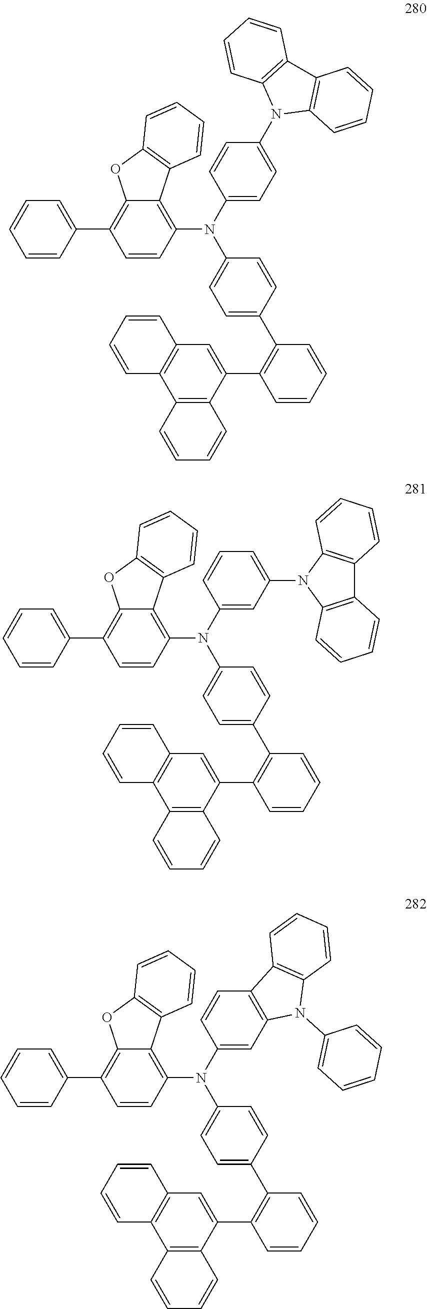 [SMILES: c1ccc(-c2ccc(N(c3ccc(-c4ccccc4-c4cc5ccccc5c5ccccc45)cc3)c3ccc(-n4c5ccccc5c5ccccc54)cc3)c3c2oc2ccccc23)cc1 ; c1ccc(-c2ccc(N(c3ccc(-c4ccccc4-c4cc5ccccc5c5ccccc45)cc3)c3cccc(-n4c5ccccc5c5ccccc54)c3)c3c2oc2ccccc23)cc1 ; c1ccc(-c2ccc(N(c3ccc(-c4ccccc4-c4cc5ccccc5c5ccccc45)cc3)c3ccc4c5ccccc5n(-c5ccccc5)c4c3)c3c2oc2ccccc23)cc1]

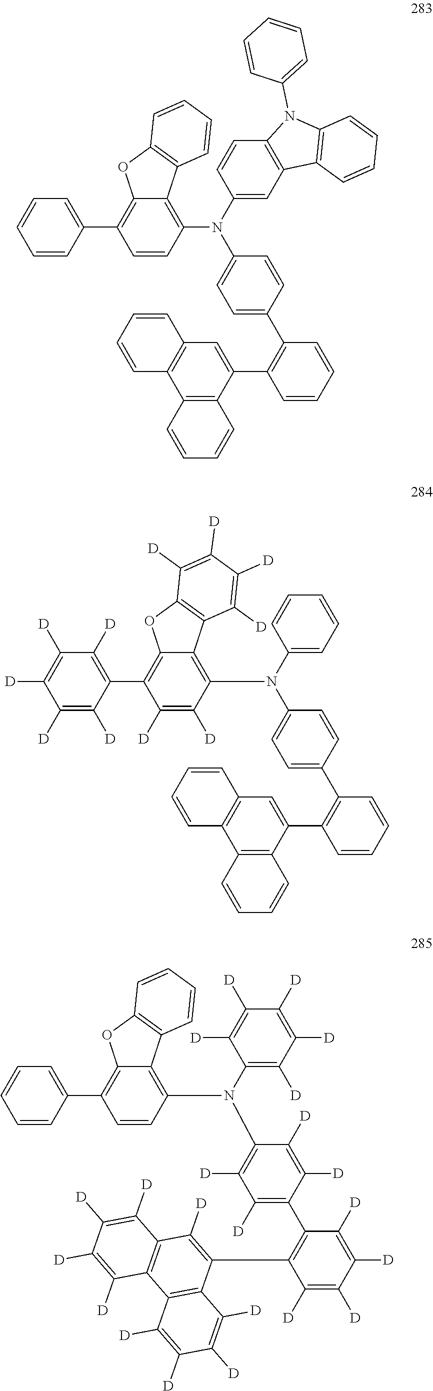 [SMILES: c1ccc(-c2ccc(N(c3ccc(-c4ccccc4-c4cc5ccccc5c5ccccc45)cc3)c3ccc4c(c3)c3ccccc3n4-c3ccccc3)c3c2oc2ccccc23)cc1 ; [2H]c1c([2H])c([2H])c(-c2c([2H])c([2H])c(N(c3ccccc3)c3ccc(-c4ccccc4-c4cc5ccccc5c5ccccc45)cc3)c3c2oc2c([2H])c([2H])c([2H])c([2H])c23)c([2H])c1[2H] ; [2H]c1c([2H])c([2H])c(N(c2c([2H])c([2H])c(-c3c([2H])c([2H])c([2H])c([2H])c3-c3c([2H])c4c([2H])c([2H])c([2H])c([2H])c4c4c([2H])c([2H])c([2H])c([2H])c34)c([2H])c2[2H])c2ccc(-c3ccccc3)c3oc4ccccc4c23)c([2H])c1[2H]]

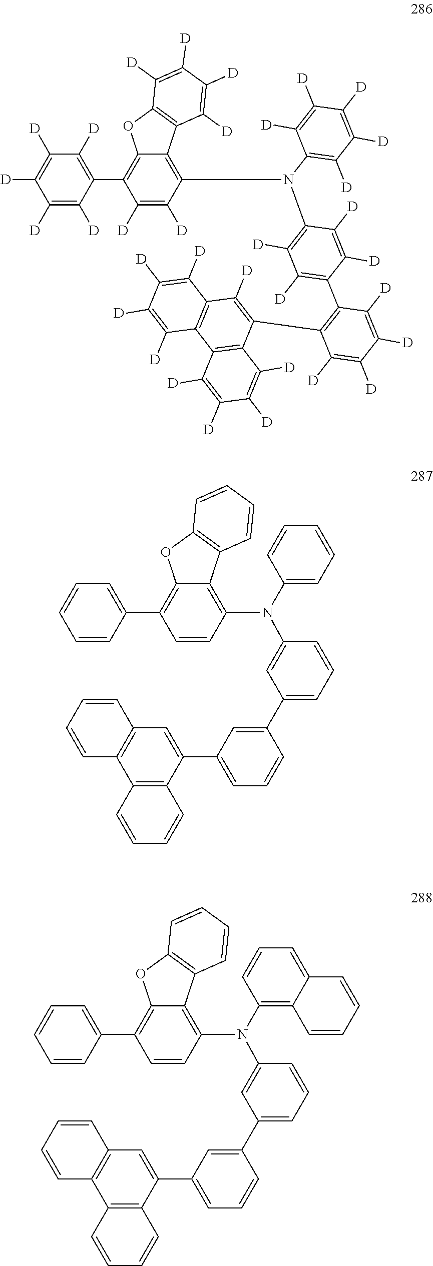 [SMILES: [2H]c1c([2H])c([2H])c(-c2c([2H])c([2H])c(N(c3c([2H])c([2H])c([2H])c([2H])c3[2H])c3c([2H])c([2H])c(-c4c([2H])c([2H])c([2H])c([2H])c4-c4c([2H])c5c([2H])c([2H])c([2H])c([2H])c5c5c([2H])c([2H])c([2H])c([2H])c45)c([2H])c3[2H])c3c2oc2c([2H])c([2H])c([2H])c([2H])c23)c([2H])c1[2H] ; c1ccc(-c2ccc(N(c3ccccc3)c3cccc(-c4cccc(-c5cc6ccccc6c6ccccc56)c4)c3)c3c2oc2ccccc23)cc1 ; c1ccc(-c2ccc(N(c3cccc(-c4cccc(-c5cc6ccccc6c6ccccc56)c4)c3)c3cccc4ccccc34)c3c2oc2ccccc23)cc1]

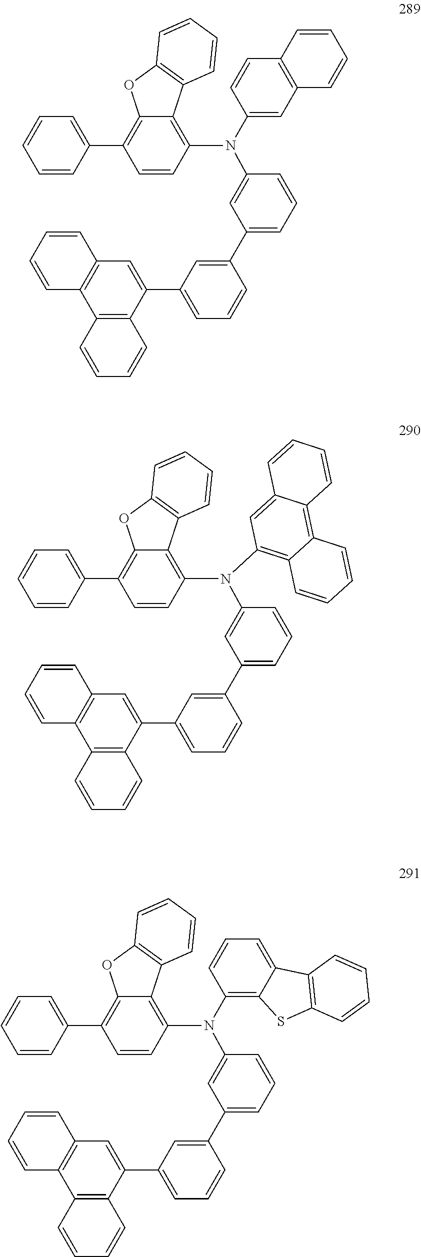 [SMILES: c1ccc(-c2ccc(N(c3cccc(-c4cccc(-c5cc6ccccc6c6ccccc56)c4)c3)c3ccc4ccccc4c3)c3c2oc2ccccc23)cc1 ; c1ccc(-c2ccc(N(c3cccc(-c4cccc(-c5cc6ccccc6c6ccccc56)c4)c3)c3cc4ccccc4c4ccccc34)c3c2oc2ccccc23)cc1 ; c1ccc(-c2ccc(N(c3cccc(-c4cccc(-c5cc6ccccc6c6ccccc56)c4)c3)c3cccc4c3sc3ccccc34)c3c2oc2ccccc23)cc1]

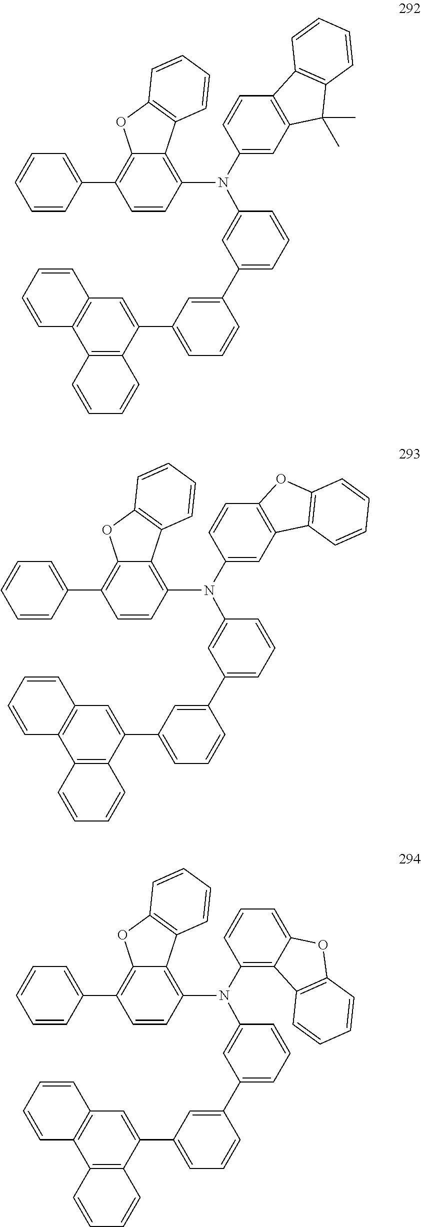 [SMILES: CC1(C)c2ccccc2-c2ccc(N(c3cccc(-c4cccc(-c5cc6ccccc6c6ccccc56)c4)c3)c3ccc(-c4ccccc4)c4oc5ccccc5c34)cc21 ; c1ccc(-c2ccc(N(c3cccc(-c4cccc(-c5cc6ccccc6c6ccccc56)c4)c3)c3ccc4oc5ccccc5c4c3)c3c2oc2ccccc23)cc1 ; c1ccc(-c2ccc(N(c3cccc(-c4cccc(-c5cc6ccccc6c6ccccc56)c4)c3)c3cccc4oc5ccccc5c34)c3c2oc2ccccc23)cc1]

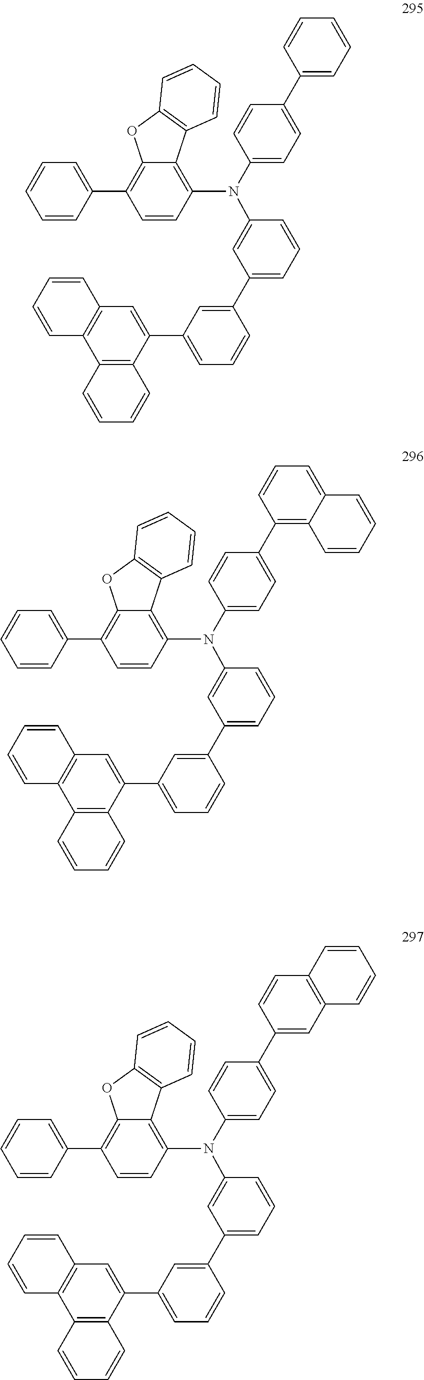 [SMILES: c1ccc(-c2ccc(N(c3cccc(-c4cccc(-c5cc6ccccc6c6ccccc56)c4)c3)c3ccc(-c4ccccc4)c4oc5ccccc5c34)cc2)cc1 ; c1ccc(-c2ccc(N(c3ccc(-c4cccc5ccccc45)cc3)c3cccc(-c4cccc(-c5cc6ccccc6c6ccccc56)c4)c3)c3c2oc2ccccc23)cc1 ; c1ccc(-c2ccc(N(c3ccc(-c4ccc5ccccc5c4)cc3)c3cccc(-c4cccc(-c5cc6ccccc6c6ccccc56)c4)c3)c3c2oc2ccccc23)cc1]

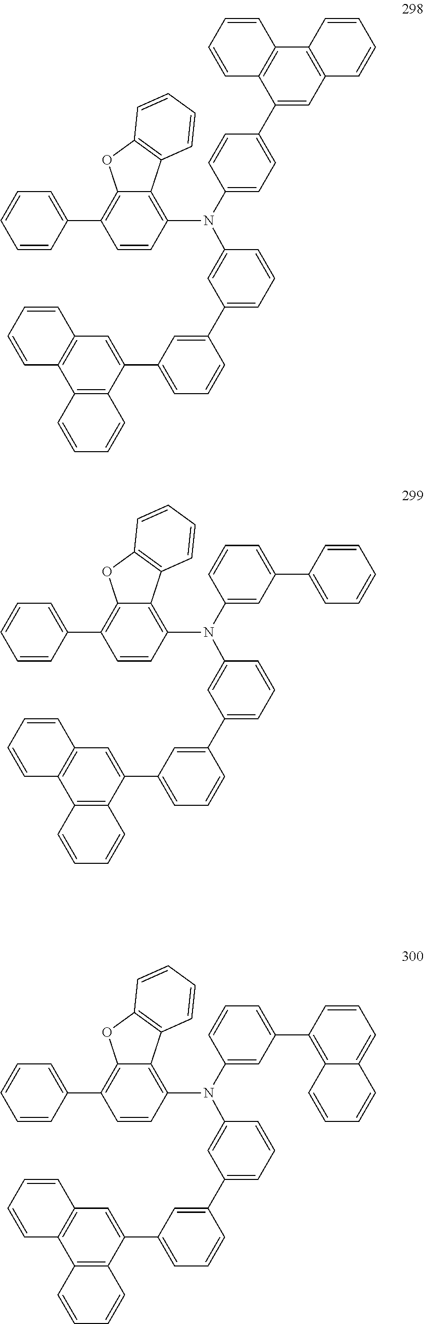 [SMILES: c1ccc(-c2ccc(N(c3ccc(-c4cc5ccccc5c5ccccc45)cc3)c3cccc(-c4cccc(-c5cc6ccccc6c6ccccc56)c4)c3)c3c2oc2ccccc23)cc1 ; c1ccc(-c2cccc(N(c3cccc(-c4cccc(-c5cc6ccccc6c6ccccc56)c4)c3)c3ccc(-c4ccccc4)c4oc5ccccc5c34)c2)cc1 ; c1ccc(-c2ccc(N(c3cccc(-c4cccc(-c5cc6ccccc6c6ccccc56)c4)c3)c3cccc(-c4cccc5ccccc45)c3)c3c2oc2ccccc23)cc1]

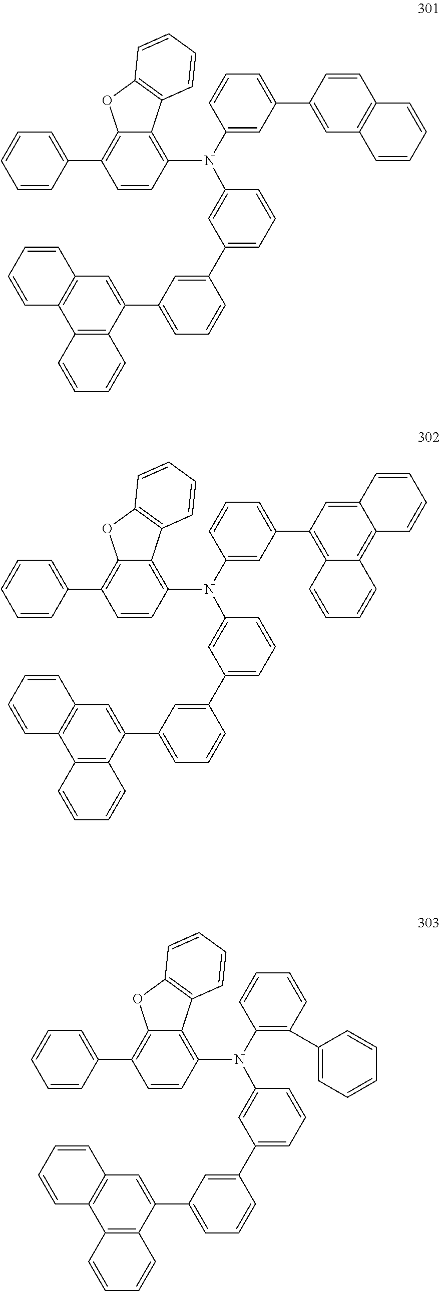 [SMILES: c1ccc(-c2ccc(N(c3cccc(-c4cccc(-c5cc6ccccc6c6ccccc56)c4)c3)c3cccc(-c4ccc5ccccc5c4)c3)c3c2oc2ccccc23)cc1 ; c1ccc(-c2ccc(N(c3cccc(-c4cccc(-c5cc6ccccc6c6ccccc56)c4)c3)c3cccc(-c4cc5ccccc5c5ccccc45)c3)c3c2oc2ccccc23)cc1 ; c1ccc(-c2ccccc2N(c2cccc(-c3cccc(-c4cc5ccccc5c5ccccc45)c3)c2)c2ccc(-c3ccccc3)c3oc4ccccc4c23)cc1]

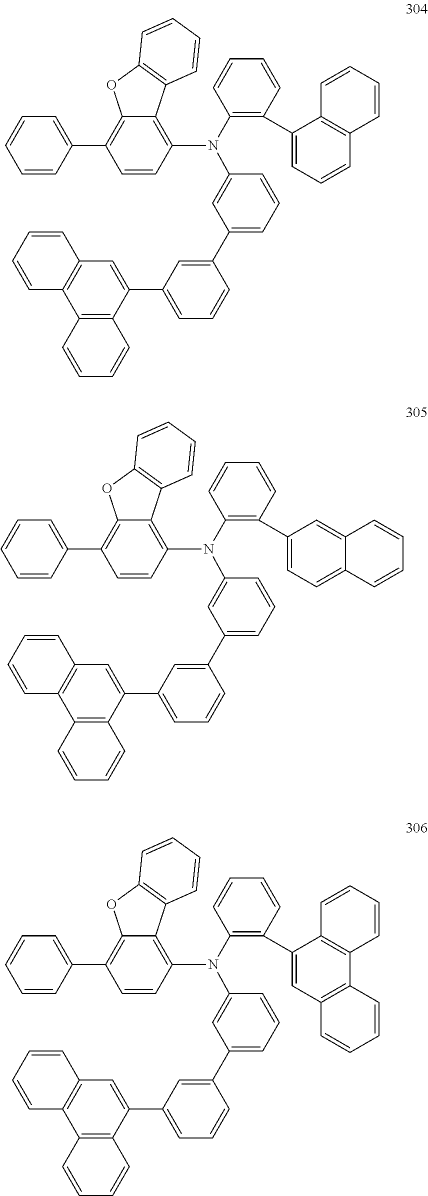 [SMILES: c1ccc(-c2ccc(N(c3cccc(-c4cccc(-c5cc6ccccc6c6ccccc56)c4)c3)c3ccccc3-c3cccc4ccccc34)c3c2oc2ccccc23)cc1 ; c1ccc(-c2ccc(N(c3cccc(-c4cccc(-c5cc6ccccc6c6ccccc56)c4)c3)c3ccccc3-c3ccc4ccccc4c3)c3c2oc2ccccc23)cc1 ; c1ccc(-c2ccc(N(c3cccc(-c4cccc(-c5cc6ccccc6c6ccccc56)c4)c3)c3ccccc3-c3cc4ccccc4c4ccccc34)c3c2oc2ccccc23)cc1]

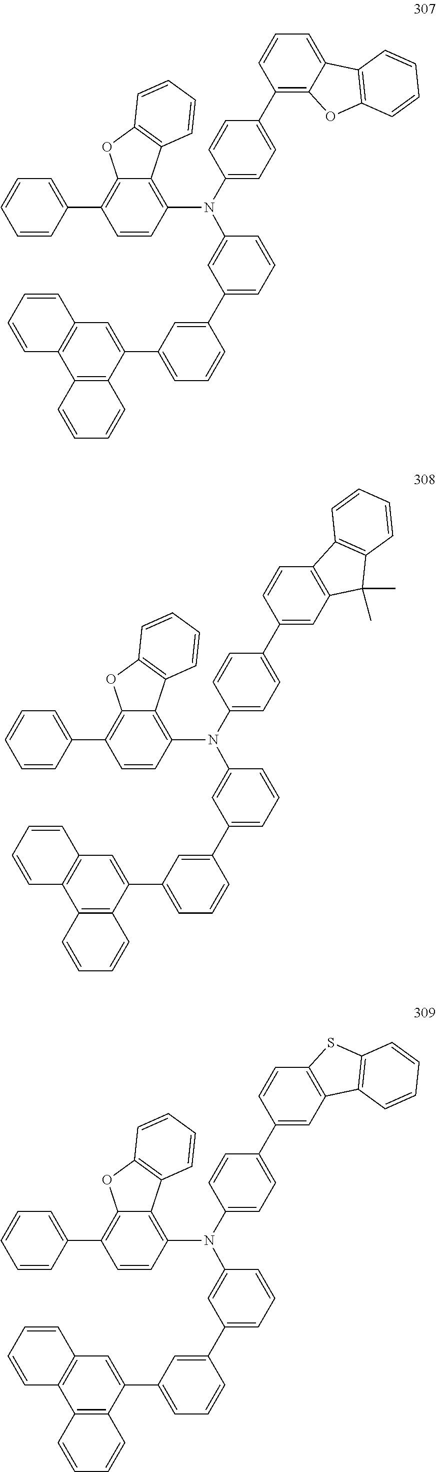 [SMILES: c1ccc(-c2ccc(N(c3ccc(-c4cccc5c4oc4ccccc45)cc3)c3cccc(-c4cccc(-c5cc6ccccc6c6ccccc56)c4)c3)c3c2oc2ccccc23)cc1 ; CC1(C)c2ccccc2-c2ccc(-c3ccc(N(c4cccc(-c5cccc(-c6cc7ccccc7c7ccccc67)c5)c4)c4ccc(-c5ccccc5)c5oc6ccccc6c45)cc3)cc21 ; c1ccc(-c2ccc(N(c3ccc(-c4ccc5sc6ccccc6c5c4)cc3)c3cccc(-c4cccc(-c5cc6ccccc6c6ccccc56)c4)c3)c3c2oc2ccccc23)cc1]

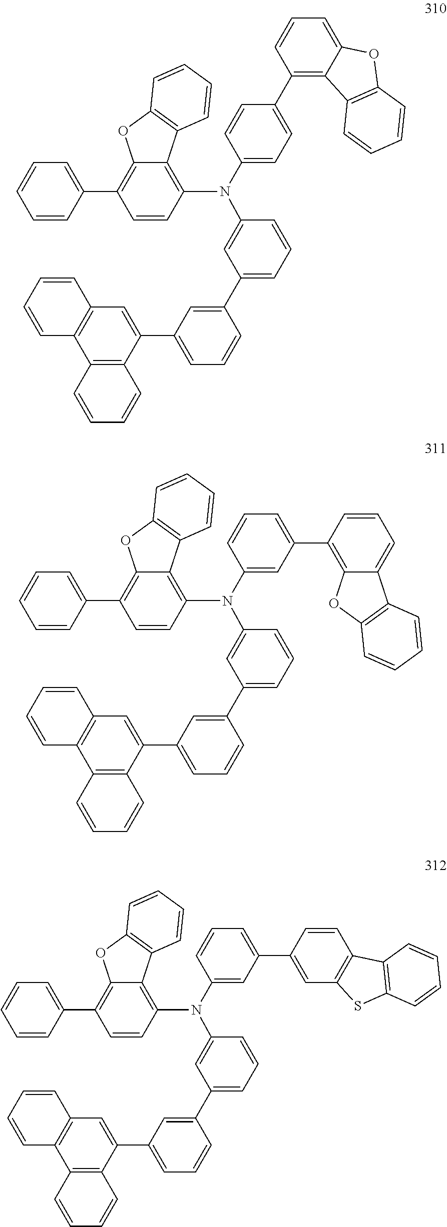 [SMILES: c1ccc(-c2ccc(N(c3ccc(-c4cccc5oc6ccccc6c45)cc3)c3cccc(-c4cccc(-c5cc6ccccc6c6ccccc56)c4)c3)c3c2oc2ccccc23)cc1 ; c1ccc(-c2ccc(N(c3cccc(-c4cccc(-c5cc6ccccc6c6ccccc56)c4)c3)c3cccc(-c4cccc5c4oc4ccccc45)c3)c3c2oc2ccccc23)cc1 ; c1ccc(-c2ccc(N(c3cccc(-c4cccc(-c5cc6ccccc6c6ccccc56)c4)c3)c3cccc(-c4ccc5c(c4)sc4ccccc45)c3)c3c2oc2ccccc23)cc1]

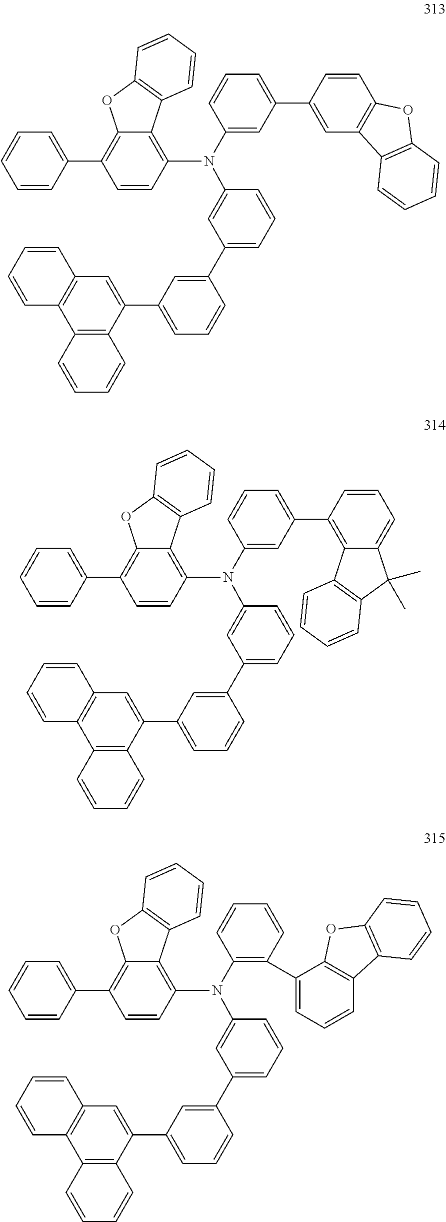 [SMILES: c1ccc(-c2ccc(N(c3cccc(-c4cccc(-c5cc6ccccc6c6ccccc56)c4)c3)c3cccc(-c4ccc5oc6ccccc6c5c4)c3)c3c2oc2ccccc23)cc1 ; CC1(C)c2ccccc2-c2c(-c3cccc(N(c4cccc(-c5cccc(-c6cc7ccccc7c7ccccc67)c5)c4)c4ccc(-c5ccccc5)c5oc6ccccc6c45)c3)cccc21 ; c1ccc(-c2ccc(N(c3cccc(-c4cccc(-c5cc6ccccc6c6ccccc56)c4)c3)c3ccccc3-c3cccc4c3oc3ccccc34)c3c2oc2ccccc23)cc1]

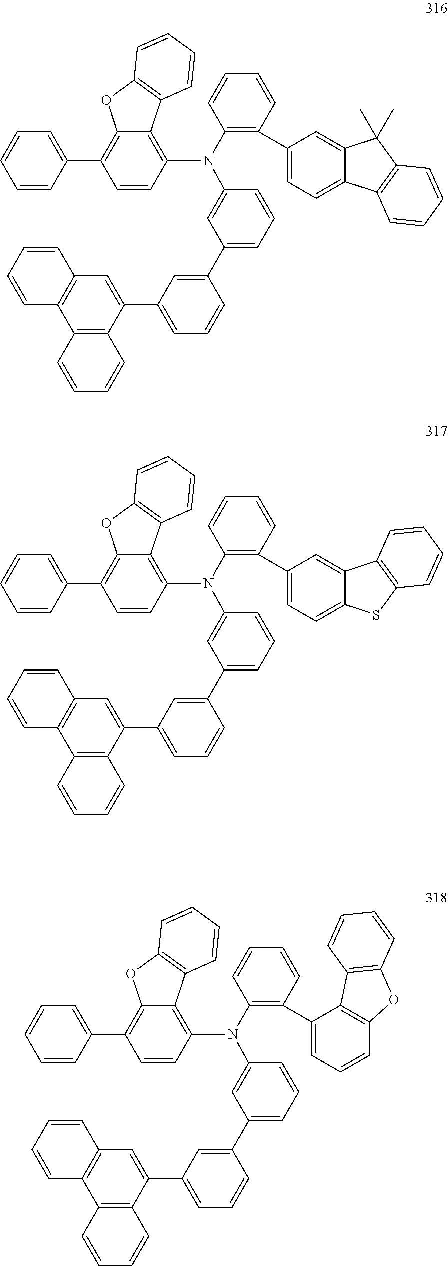 [SMILES: CC1(C)c2ccccc2-c2ccc(-c3ccccc3N(c3cccc(-c4cccc(-c5cc6ccccc6c6ccccc56)c4)c3)c3ccc(-c4ccccc4)c4oc5ccccc5c34)cc21 ; c1ccc(-c2ccc(N(c3cccc(-c4cccc(-c5cc6ccccc6c6ccccc56)c4)c3)c3ccccc3-c3ccc4sc5ccccc5c4c3)c3c2oc2ccccc23)cc1 ; c1ccc(-c2ccc(N(c3cccc(-c4cccc(-c5cc6ccccc6c6ccccc56)c4)c3)c3ccccc3-c3cccc4oc5ccccc5c34)c3c2oc2ccccc23)cc1]

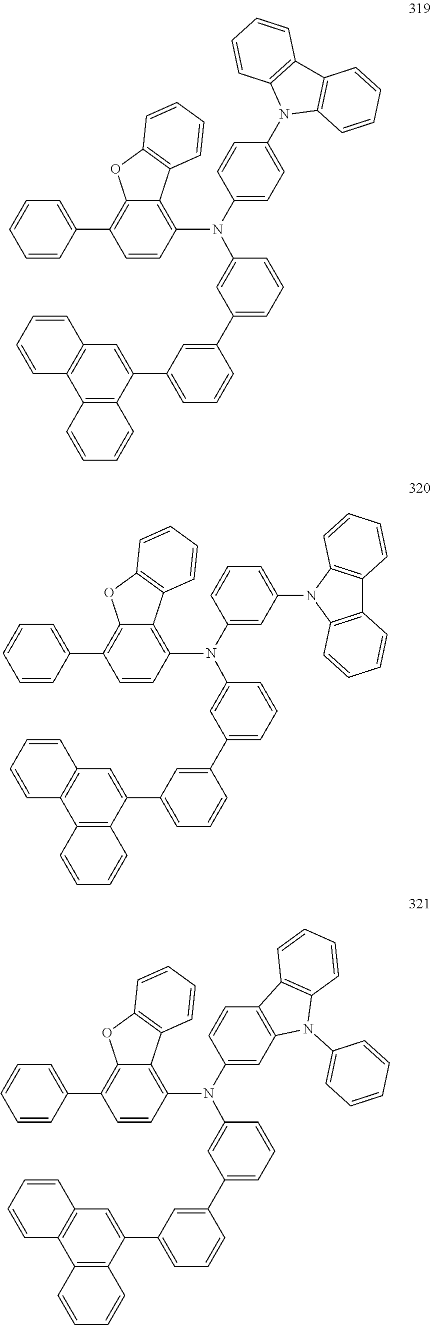 [SMILES: c1ccc(-c2ccc(N(c3ccc(-n4c5ccccc5c5ccccc54)cc3)c3cccc(-c4cccc(-c5cc6ccccc6c6ccccc56)c4)c3)c3c2oc2ccccc23)cc1 ; c1ccc(-c2ccc(N(c3cccc(-c4cccc(-c5cc6ccccc6c6ccccc56)c4)c3)c3cccc(-n4c5ccccc5c5ccccc54)c3)c3c2oc2ccccc23)cc1 ; c1ccc(-c2ccc(N(c3cccc(-c4cccc(-c5cc6ccccc6c6ccccc56)c4)c3)c3ccc4c5ccccc5n(-c5ccccc5)c4c3)c3c2oc2ccccc23)cc1]

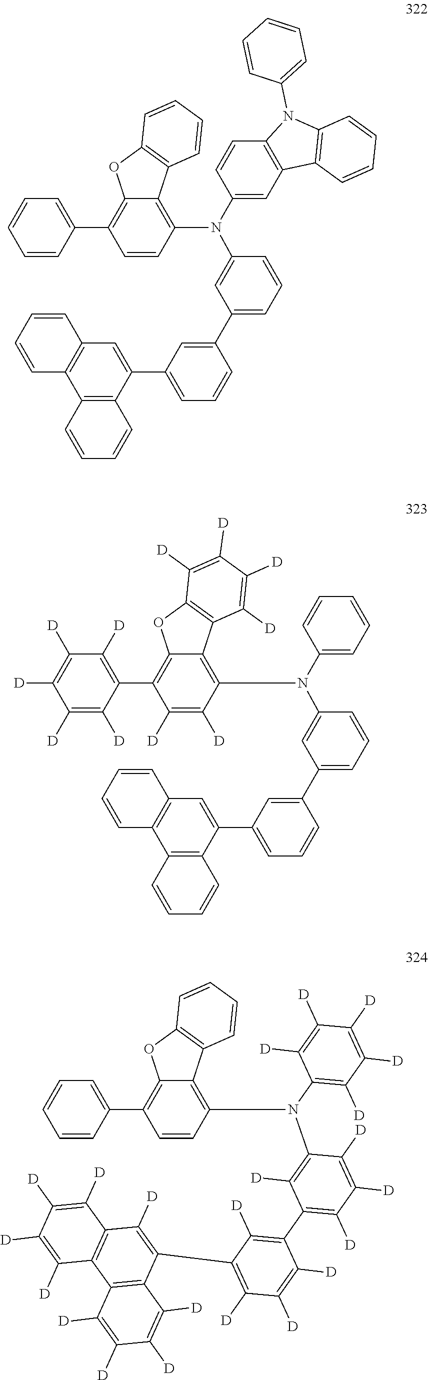 [SMILES: c1ccc(-c2ccc(N(c3cccc(-c4cccc(-c5cc6ccccc6c6ccccc56)c4)c3)c3ccc4c(c3)c3ccccc3n4-c3ccccc3)c3c2oc2ccccc23)cc1 ; [2H]c1c([2H])c([2H])c(-c2c([2H])c([2H])c(N(c3ccccc3)c3cccc(-c4cccc(-c5cc6ccccc6c6ccccc56)c4)c3)c3c2oc2c([2H])c([2H])c([2H])c([2H])c23)c([2H])c1[2H] ; [2H]c1c([2H])c([2H])c(N(c2c([2H])c([2H])c([2H])c(-c3c([2H])c([2H])c([2H])c(-c4c([2H])c5c([2H])c([2H])c([2H])c([2H])c5c5c([2H])c([2H])c([2H])c([2H])c45)c3[2H])c2[2H])c2ccc(-c3ccccc3)c3oc4ccccc4c23)c([2H])c1[2H]]

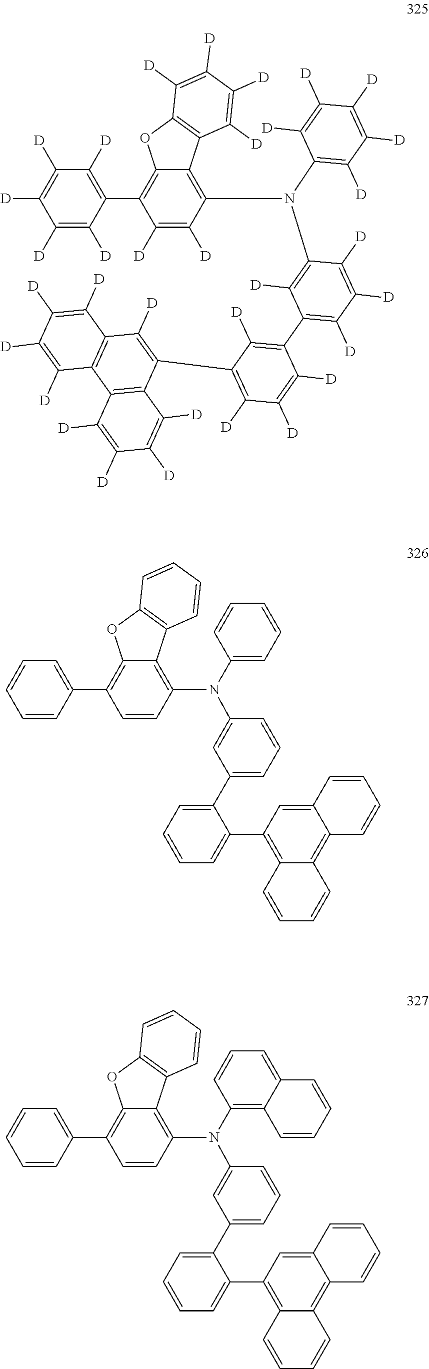 [SMILES: [2H]c1c([2H])c([2H])c(-c2c([2H])c([2H])c(N(c3c([2H])c([2H])c([2H])c([2H])c3[2H])c3c([2H])c([2H])c([2H])c(-c4c([2H])c([2H])c([2H])c(-c5c([2H])c6c([2H])c([2H])c([2H])c([2H])c6c6c([2H])c([2H])c([2H])c([2H])c56)c4[2H])c3[2H])c3c2oc2c([2H])c([2H])c([2H])c([2H])c23)c([2H])c1[2H] ; c1ccc(-c2ccc(N(c3ccccc3)c3cccc(-c4ccccc4-c4cc5ccccc5c5ccccc45)c3)c3c2oc2ccccc23)cc1 ; c1ccc(-c2ccc(N(c3cccc(-c4ccccc4-c4cc5ccccc5c5ccccc45)c3)c3cccc4ccccc34)c3c2oc2ccccc23)cc1]

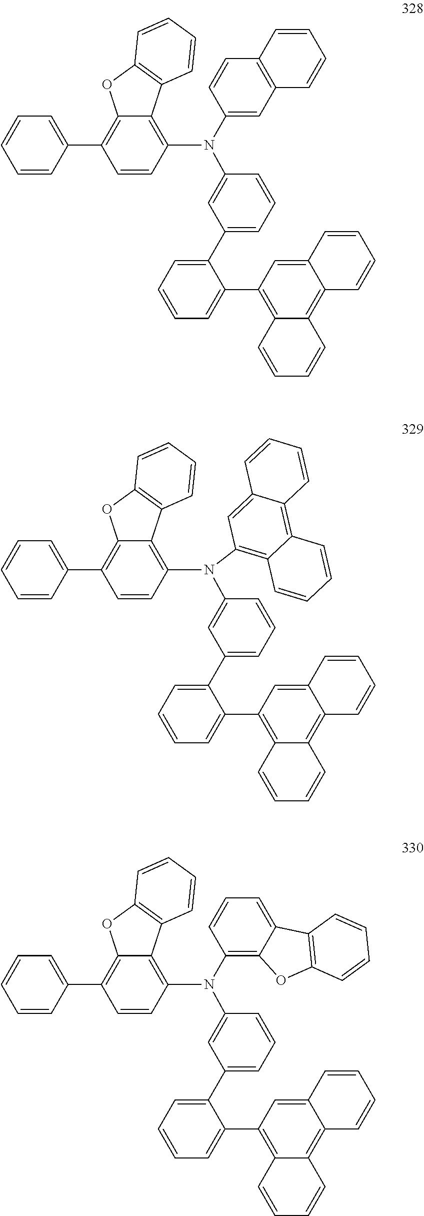 [SMILES: c1ccc(-c2ccc(N(c3cccc(-c4ccccc4-c4cc5ccccc5c5ccccc45)c3)c3ccc4ccccc4c3)c3c2oc2ccccc23)cc1 ; c1ccc(-c2ccc(N(c3cccc(-c4ccccc4-c4cc5ccccc5c5ccccc45)c3)c3cc4ccccc4c4ccccc34)c3c2oc2ccccc23)cc1 ; c1ccc(-c2ccc(N(c3cccc(-c4ccccc4-c4cc5ccccc5c5ccccc45)c3)c3cccc4c3oc3ccccc34)c3c2oc2ccccc23)cc1]

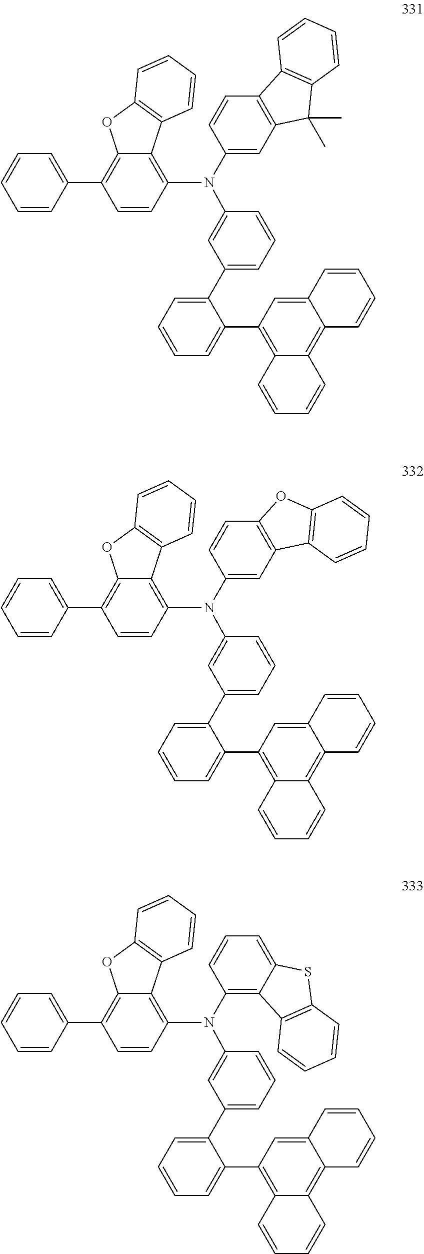 [SMILES: CC1(C)c2ccccc2-c2ccc(N(c3cccc(-c4ccccc4-c4cc5ccccc5c5ccccc45)c3)c3ccc(-c4ccccc4)c4oc5ccccc5c34)cc21 ; c1ccc(-c2ccc(N(c3cccc(-c4ccccc4-c4cc5ccccc5c5ccccc45)c3)c3ccc4oc5ccccc5c4c3)c3c2oc2ccccc23)cc1 ; c1ccc(-c2ccc(N(c3cccc(-c4ccccc4-c4cc5ccccc5c5ccccc45)c3)c3cccc4sc5ccccc5c34)c3c2oc2ccccc23)cc1]

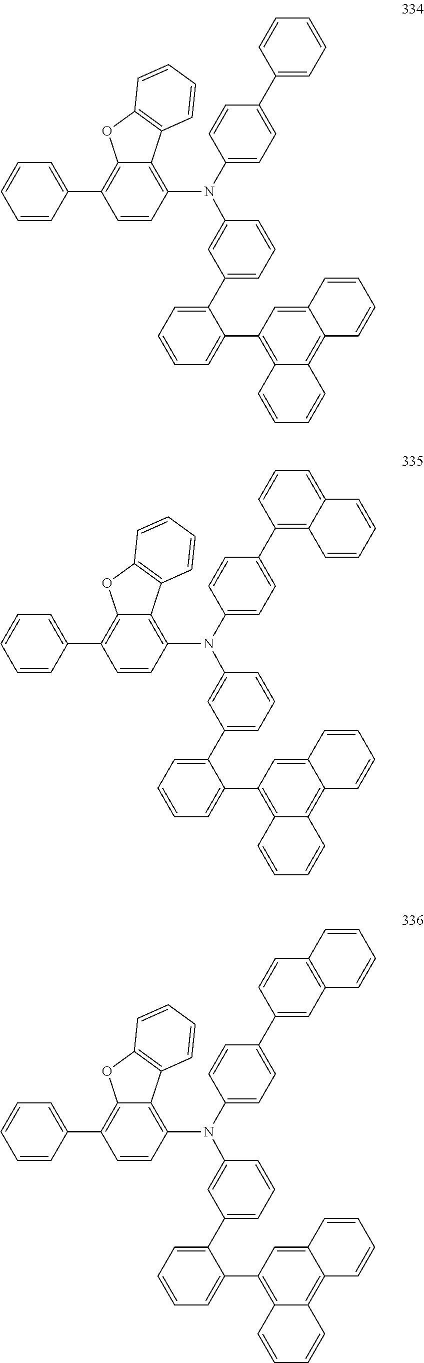 [SMILES: c1ccc(-c2ccc(N(c3cccc(-c4ccccc4-c4cc5ccccc5c5ccccc45)c3)c3ccc(-c4ccccc4)c4oc5ccccc5c34)cc2)cc1 ; c1ccc(-c2ccc(N(c3ccc(-c4cccc5ccccc45)cc3)c3cccc(-c4ccccc4-c4cc5ccccc5c5ccccc45)c3)c3c2oc2ccccc23)cc1 ; c1ccc(-c2ccc(N(c3ccc(-c4ccc5ccccc5c4)cc3)c3cccc(-c4ccccc4-c4cc5ccccc5c5ccccc45)c3)c3c2oc2ccccc23)cc1]

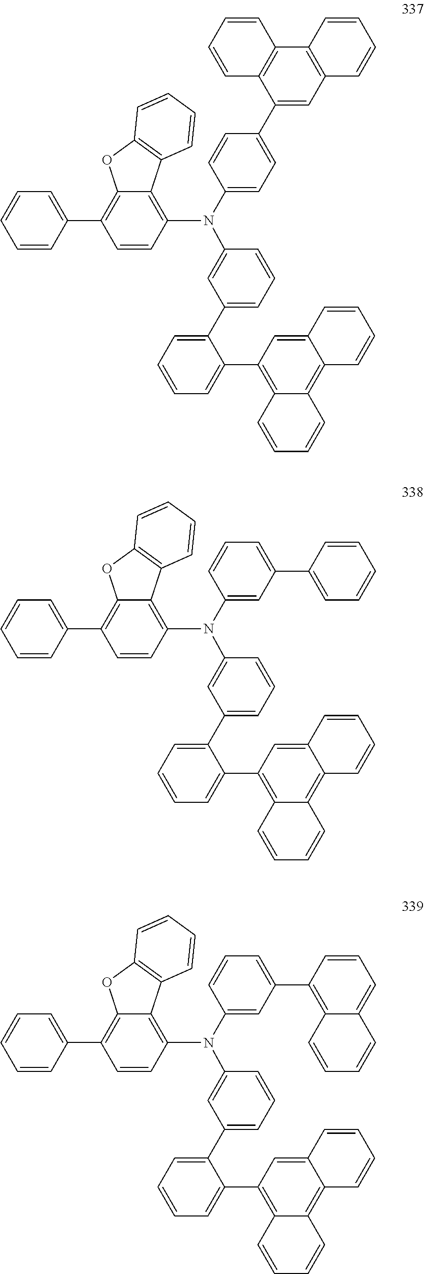 [SMILES: c1ccc(-c2ccc(N(c3ccc(-c4cc5ccccc5c5ccccc45)cc3)c3cccc(-c4ccccc4-c4cc5ccccc5c5ccccc45)c3)c3c2oc2ccccc23)cc1 ; c1ccc(-c2cccc(N(c3cccc(-c4ccccc4-c4cc5ccccc5c5ccccc45)c3)c3ccc(-c4ccccc4)c4oc5ccccc5c34)c2)cc1 ; c1ccc(-c2ccc(N(c3cccc(-c4ccccc4-c4cc5ccccc5c5ccccc45)c3)c3cccc(-c4cccc5ccccc45)c3)c3c2oc2ccccc23)cc1]

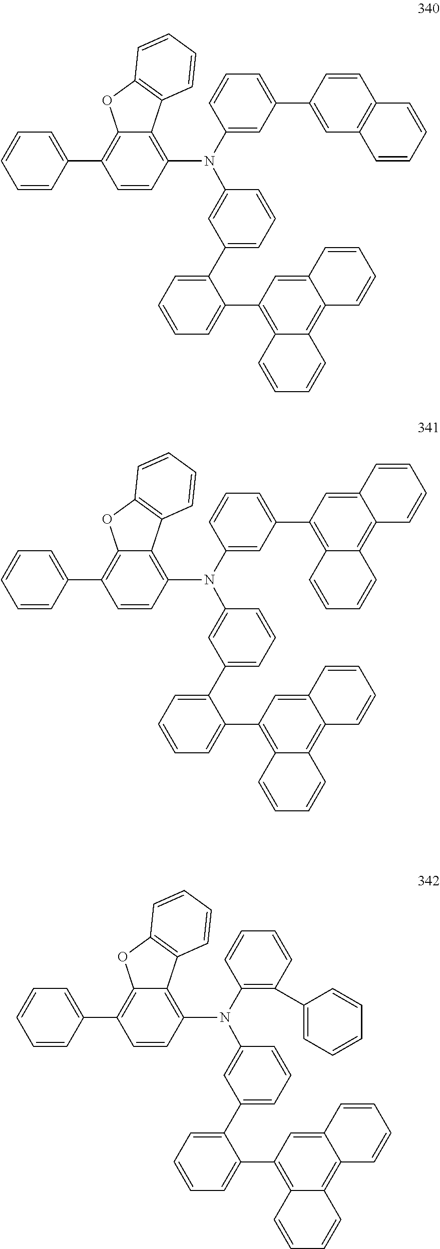 [SMILES: c1ccc(-c2ccc(N(c3cccc(-c4ccc5ccccc5c4)c3)c3cccc(-c4ccccc4-c4cc5ccccc5c5ccccc45)c3)c3c2oc2ccccc23)cc1 ; c1ccc(-c2ccc(N(c3cccc(-c4ccccc4-c4cc5ccccc5c5ccccc45)c3)c3cccc(-c4cc5ccccc5c5ccccc45)c3)c3c2oc2ccccc23)cc1 ; c1ccc(-c2ccccc2N(c2cccc(-c3ccccc3-c3cc4ccccc4c4ccccc34)c2)c2ccc(-c3ccccc3)c3oc4ccccc4c23)cc1]

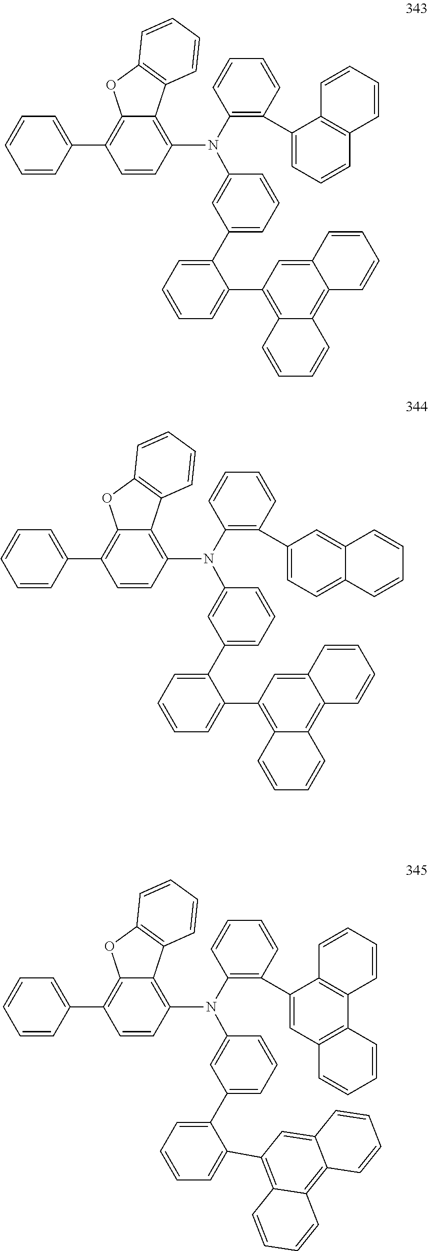 [SMILES: c1ccc(-c2ccc(N(c3cccc(-c4ccccc4-c4cc5ccccc5c5ccccc45)c3)c3ccccc3-c3cccc4ccccc34)c3c2oc2ccccc23)cc1 ; c1ccc(-c2ccc(N(c3cccc(-c4ccccc4-c4cc5ccccc5c5ccccc45)c3)c3ccccc3-c3ccc4ccccc4c3)c3c2oc2ccccc23)cc1 ; c1ccc(-c2ccc(N(c3cccc(-c4ccccc4-c4cc5ccccc5c5ccccc45)c3)c3ccccc3-c3cc4ccccc4c4ccccc34)c3c2oc2ccccc23)cc1]

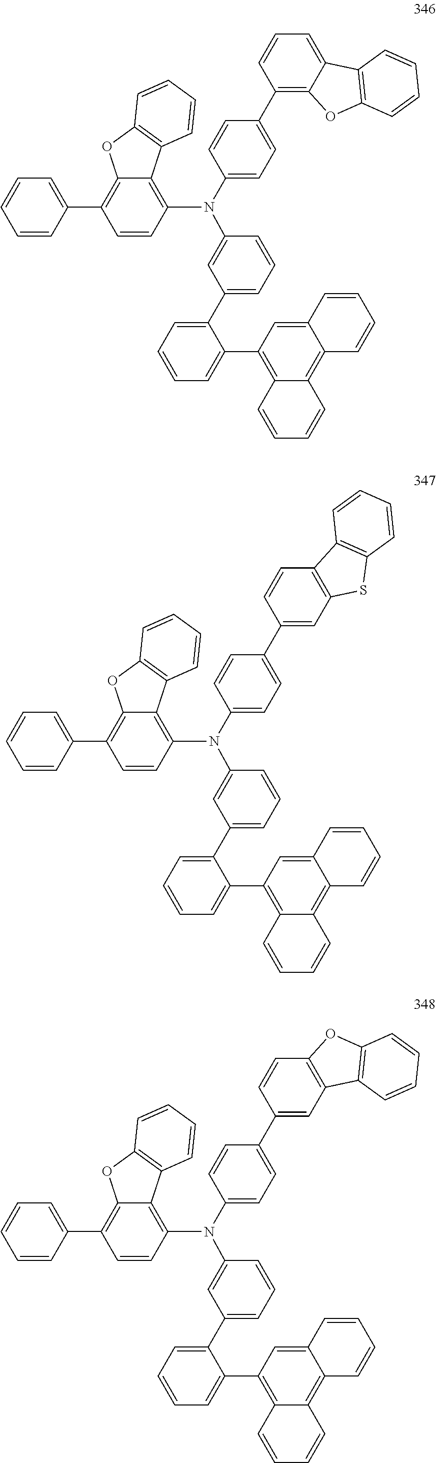 [SMILES: c1ccc(-c2ccc(N(c3ccc(-c4cccc5c4oc4ccccc45)cc3)c3cccc(-c4ccccc4-c4cc5ccccc5c5ccccc45)c3)c3c2oc2ccccc23)cc1 ; c1ccc(-c2ccc(N(c3ccc(-c4ccc5c(c4)sc4ccccc45)cc3)c3cccc(-c4ccccc4-c4cc5ccccc5c5ccccc45)c3)c3c2oc2ccccc23)cc1 ; c1ccc(-c2ccc(N(c3ccc(-c4ccc5oc6ccccc6c5c4)cc3)c3cccc(-c4ccccc4-c4cc5ccccc5c5ccccc45)c3)c3c2oc2ccccc23)cc1]

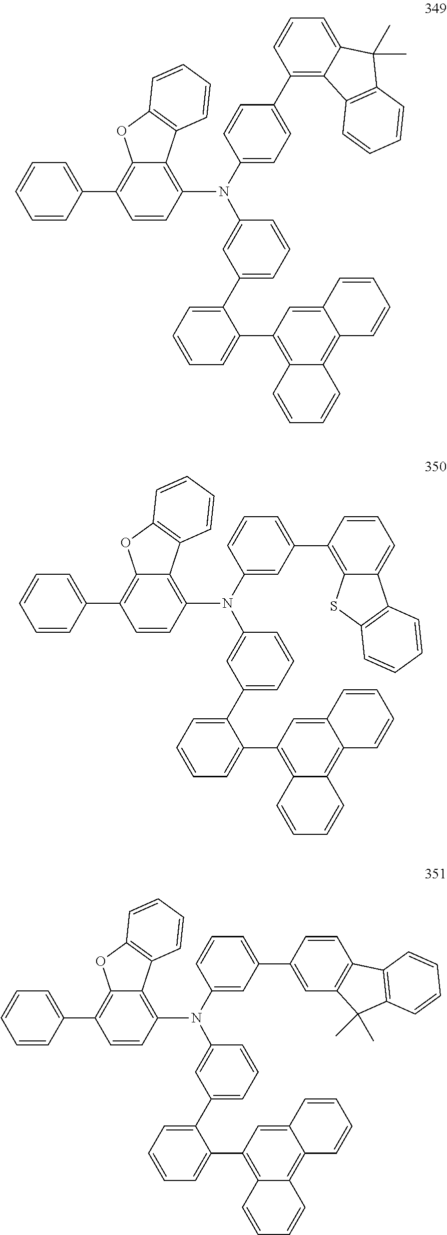 [SMILES: CC1(C)c2ccccc2-c2c(-c3ccc(N(c4cccc(-c5ccccc5-c5cc6ccccc6c6ccccc56)c4)c4ccc(-c5ccccc5)c5oc6ccccc6c45)cc3)cccc21 ; c1ccc(-c2ccc(N(c3cccc(-c4ccccc4-c4cc5ccccc5c5ccccc45)c3)c3cccc(-c4cccc5c4sc4ccccc45)c3)c3c2oc2ccccc23)cc1 ; CC1(C)c2ccccc2-c2ccc(-c3cccc(N(c4cccc(-c5ccccc5-c5cc6ccccc6c6ccccc56)c4)c4ccc(-c5ccccc5)c5oc6ccccc6c45)c3)cc21]

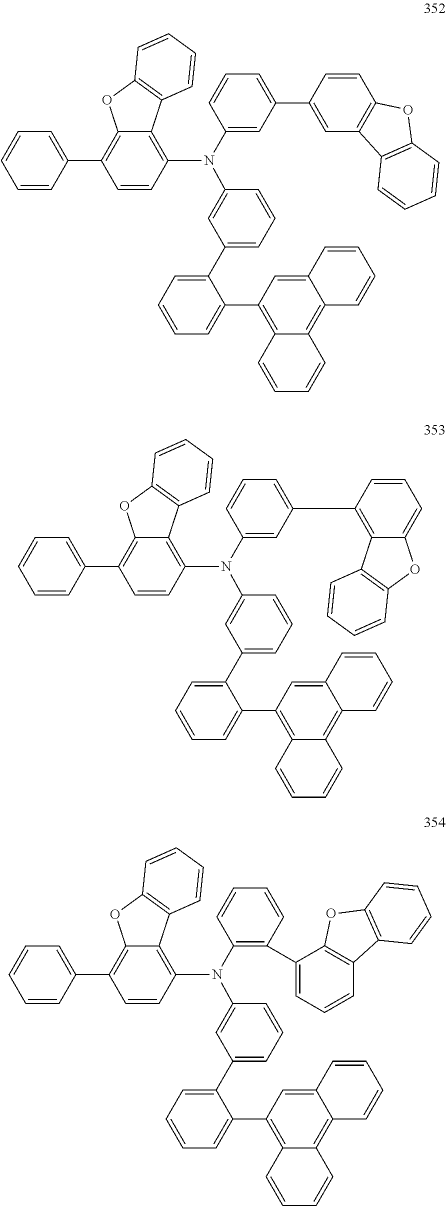 [SMILES: c1ccc(-c2ccc(N(c3cccc(-c4ccc5oc6ccccc6c5c4)c3)c3cccc(-c4ccccc4-c4cc5ccccc5c5ccccc45)c3)c3c2oc2ccccc23)cc1 ; c1ccc(-c2ccc(N(c3cccc(-c4ccccc4-c4cc5ccccc5c5ccccc45)c3)c3cccc(-c4cccc5oc6ccccc6c45)c3)c3c2oc2ccccc23)cc1 ; c1ccc(-c2ccc(N(c3cccc(-c4ccccc4-c4cc5ccccc5c5ccccc45)c3)c3ccccc3-c3cccc4c3oc3ccccc34)c3c2oc2ccccc23)cc1]

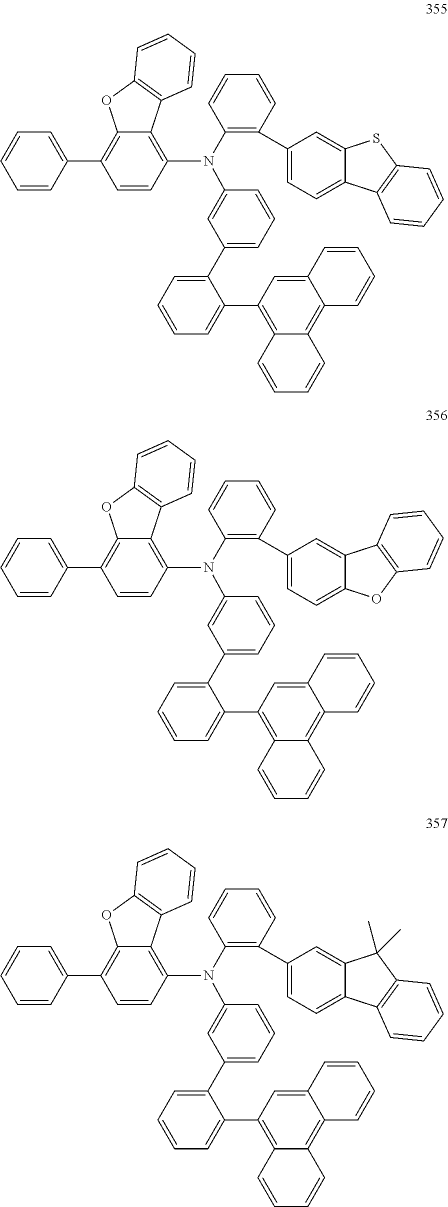 [SMILES: c1ccc(-c2ccc(N(c3cccc(-c4ccccc4-c4cc5ccccc5c5ccccc45)c3)c3ccccc3-c3ccc4c(c3)sc3ccccc34)c3c2oc2ccccc23)cc1 ; c1ccc(-c2ccc(N(c3cccc(-c4ccccc4-c4cc5ccccc5c5ccccc45)c3)c3ccccc3-c3ccc4oc5ccccc5c4c3)c3c2oc2ccccc23)cc1 ; CC1(C)c2ccccc2-c2ccc(-c3ccccc3N(c3cccc(-c4ccccc4-c4cc5ccccc5c5ccccc45)c3)c3ccc(-c4ccccc4)c4oc5ccccc5c34)cc21]

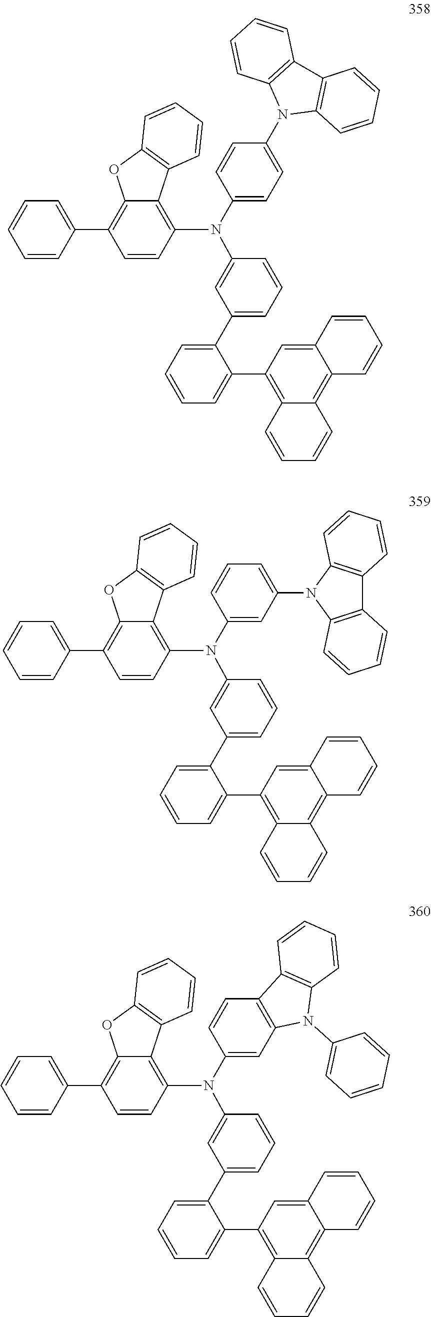

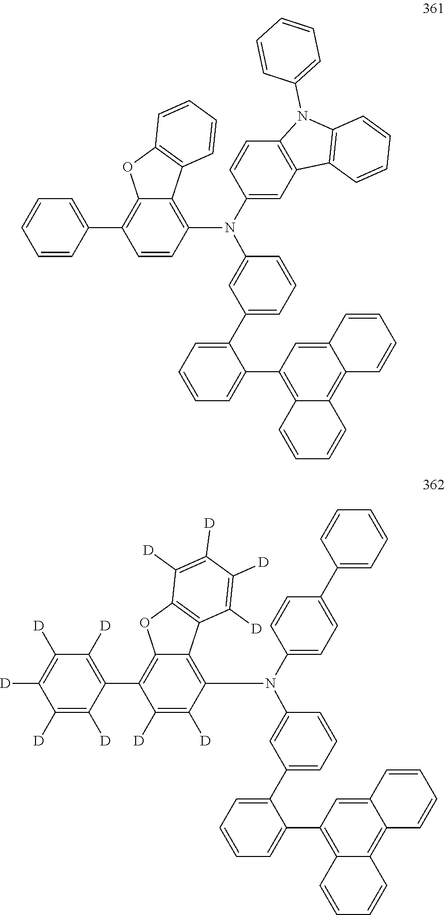 [SMILES: c1ccc(-c2ccc(N(c3cccc(-c4ccccc4-c4cc5ccccc5c5ccccc45)c3)c3ccc4c(c3)c3ccccc3n4-c3ccccc3)c3c2oc2ccccc23)cc1 ; [2H]c1c([2H])c([2H])c(-c2c([2H])c([2H])c(N(c3ccc(-c4ccccc4)cc3)c3cccc(-c4ccccc4-c4cc5ccccc5c5ccccc45)c3)c3c2oc2c([2H])c([2H])c([2H])c([2H])c23)c([2H])c1[2H]]

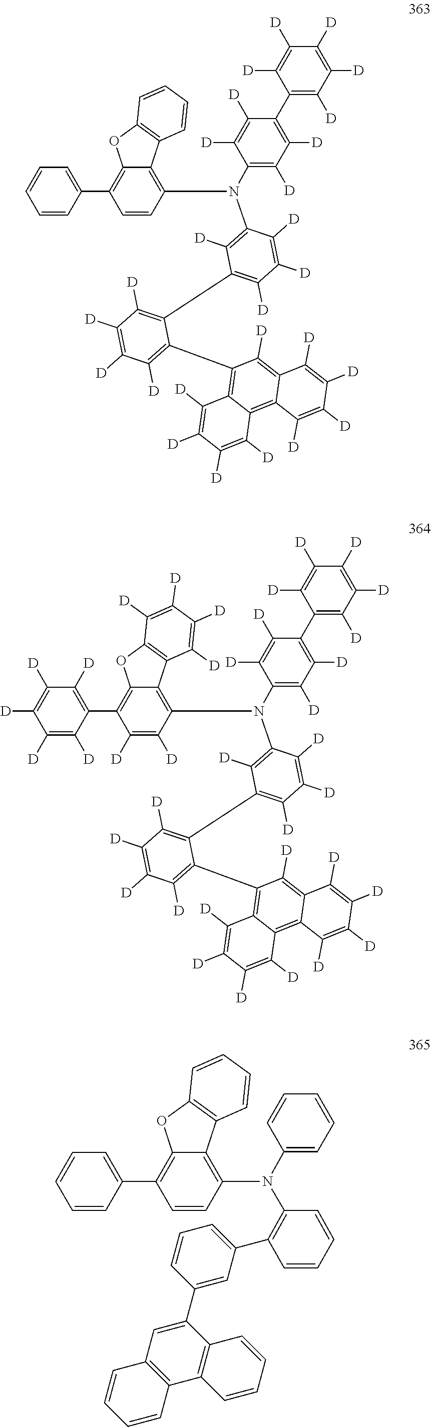

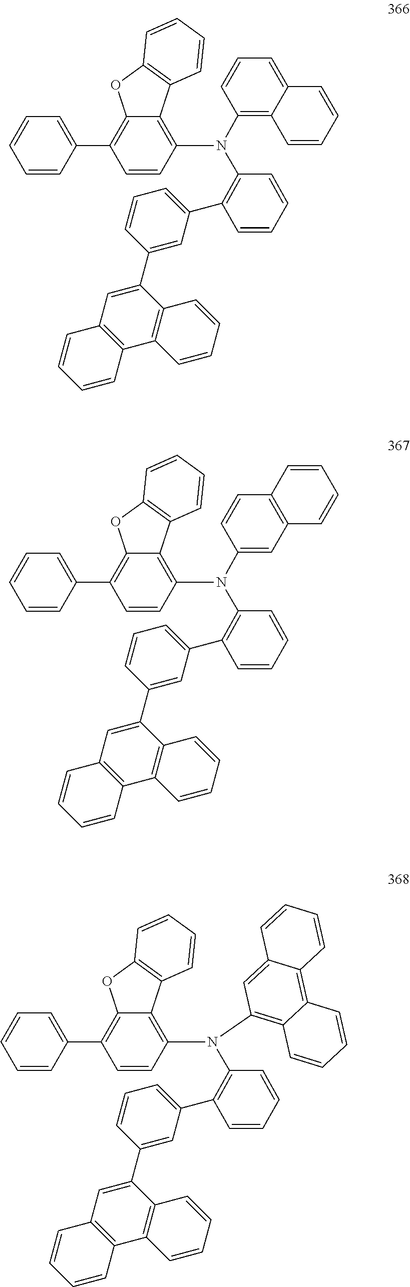 [SMILES: c1ccc(-c2ccc(N(c3ccccc3-c3cccc(-c4cc5ccccc5c5ccccc45)c3)c3cccc4ccccc34)c3c2oc2ccccc23)cc1 ; c1ccc(-c2ccc(N(c3ccc4ccccc4c3)c3ccccc3-c3cccc(-c4cc5ccccc5c5ccccc45)c3)c3c2oc2ccccc23)cc1 ; c1ccc(-c2ccc(N(c3ccccc3-c3cccc(-c4cc5ccccc5c5ccccc45)c3)c3cc4ccccc4c4ccccc34)c3c2oc2ccccc23)cc1]

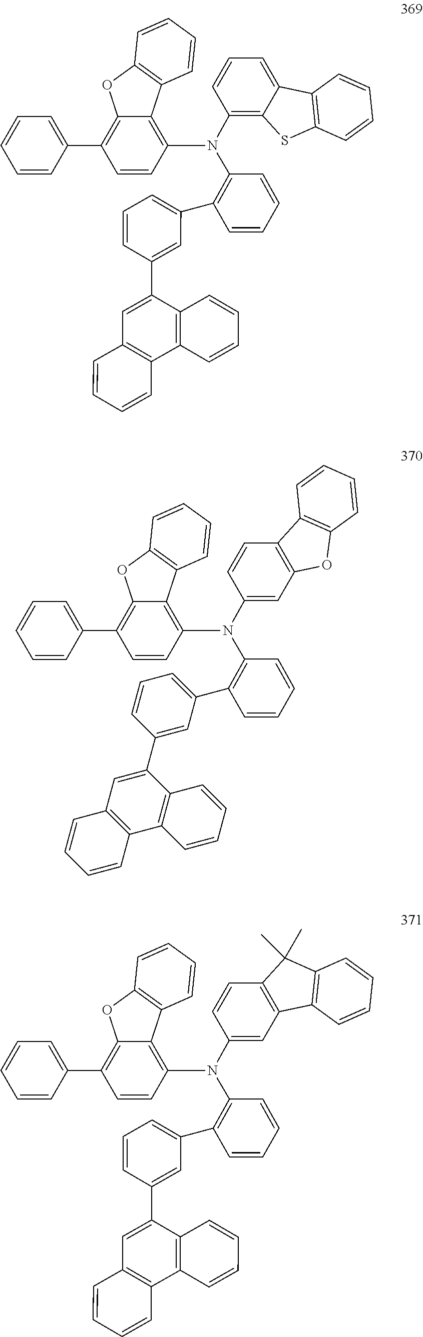 [SMILES: c1ccc(-c2ccc(N(c3ccccc3-c3cccc(-c4cc5ccccc5c5ccccc45)c3)c3cccc4c3sc3ccccc34)c3c2oc2ccccc23)cc1 ; c1ccc(-c2ccc(N(c3ccc4c(c3)oc3ccccc34)c3ccccc3-c3cccc(-c4cc5ccccc5c5ccccc45)c3)c3c2oc2ccccc23)cc1 ; CC1(C)c2ccccc2-c2cc(N(c3ccccc3-c3cccc(-c4cc5ccccc5c5ccccc45)c3)c3ccc(-c4ccccc4)c4oc5ccccc5c34)ccc21]

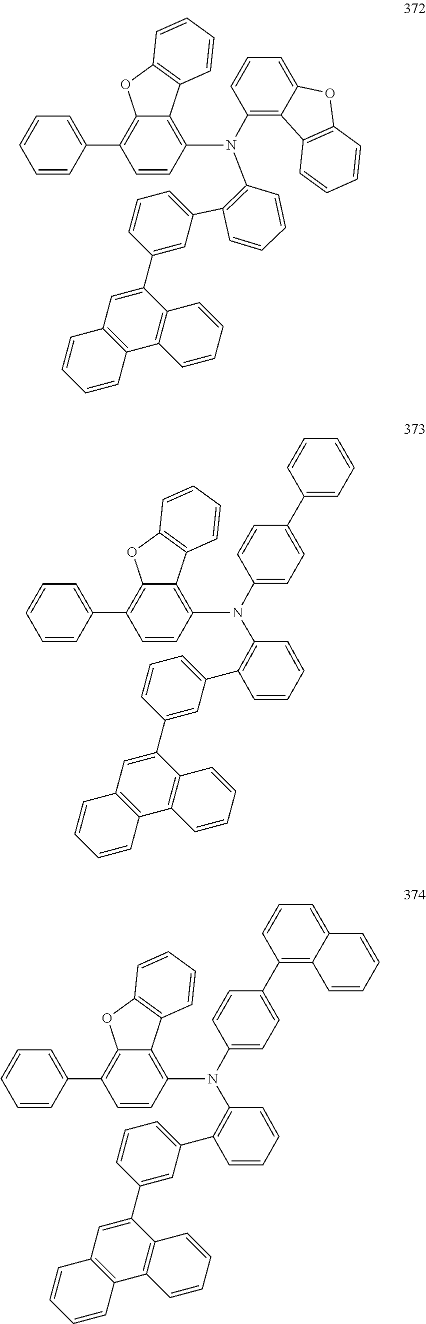 [SMILES: c1ccc(-c2ccc(N(c3ccccc3-c3cccc(-c4cc5ccccc5c5ccccc45)c3)c3cccc4oc5ccccc5c34)c3c2oc2ccccc23)cc1 ; c1ccc(-c2ccc(N(c3ccccc3-c3cccc(-c4cc5ccccc5c5ccccc45)c3)c3ccc(-c4ccccc4)c4oc5ccccc5c34)cc2)cc1 ; c1ccc(-c2ccc(N(c3ccc(-c4cccc5ccccc45)cc3)c3ccccc3-c3cccc(-c4cc5ccccc5c5ccccc45)c3)c3c2oc2ccccc23)cc1]

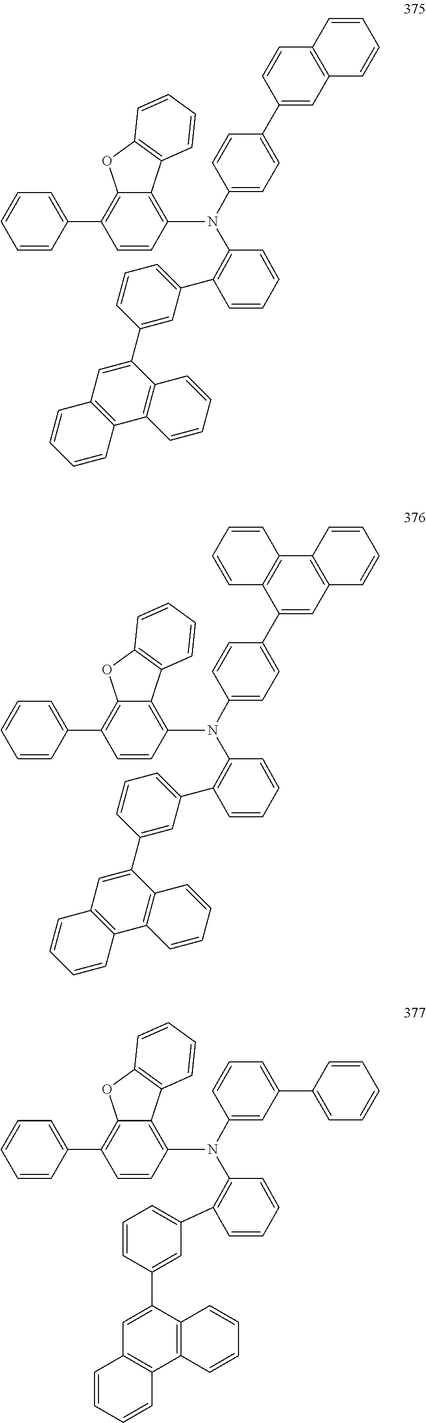 [SMILES: c1ccc(-c2ccc(N(c3ccc(-c4ccc5ccccc5c4)cc3)c3ccccc3-c3cccc(-c4cc5ccccc5c5ccccc45)c3)c3c2oc2ccccc23)cc1 ; c1ccc(-c2ccc(N(c3ccc(-c4cc5ccccc5c5ccccc45)cc3)c3ccccc3-c3cccc(-c4cc5ccccc5c5ccccc45)c3)c3c2oc2ccccc23)cc1 ; c1ccc(-c2cccc(N(c3ccccc3-c3cccc(-c4cc5ccccc5c5ccccc45)c3)c3ccc(-c4ccccc4)c4oc5ccccc5c34)c2)cc1]

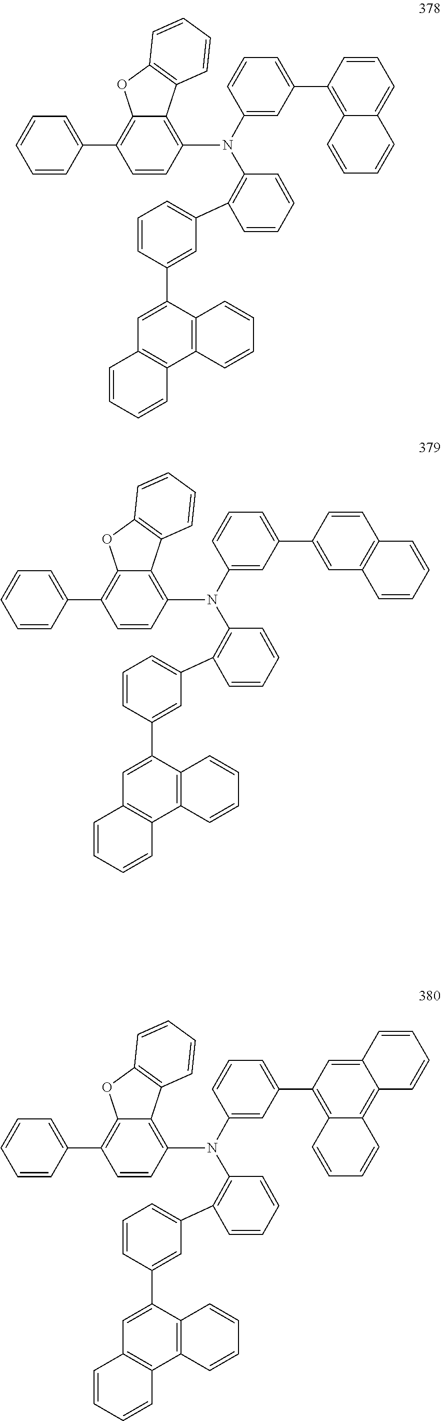 [SMILES: c1ccc(-c2ccc(N(c3cccc(-c4cccc5ccccc45)c3)c3ccccc3-c3cccc(-c4cc5ccccc5c5ccccc45)c3)c3c2oc2ccccc23)cc1 ; c1ccc(-c2ccc(N(c3cccc(-c4ccc5ccccc5c4)c3)c3ccccc3-c3cccc(-c4cc5ccccc5c5ccccc45)c3)c3c2oc2ccccc23)cc1 ; c1ccc(-c2ccc(N(c3cccc(-c4cc5ccccc5c5ccccc45)c3)c3ccccc3-c3cccc(-c4cc5ccccc5c5ccccc45)c3)c3c2oc2ccccc23)cc1]

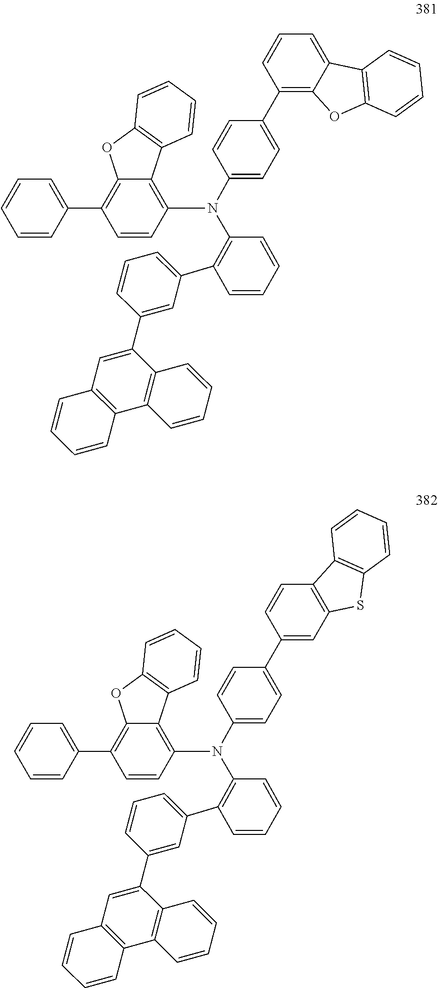

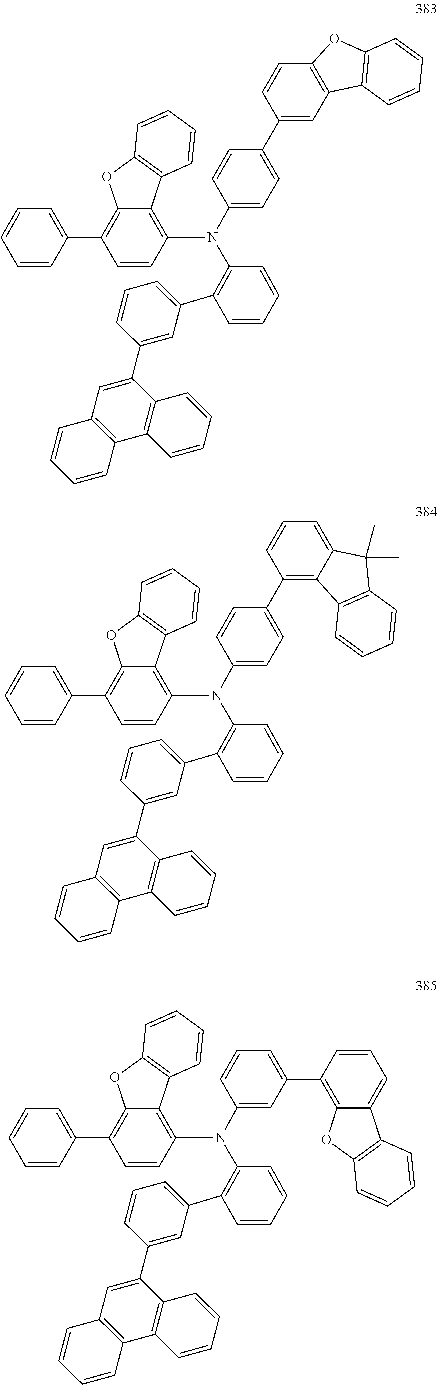

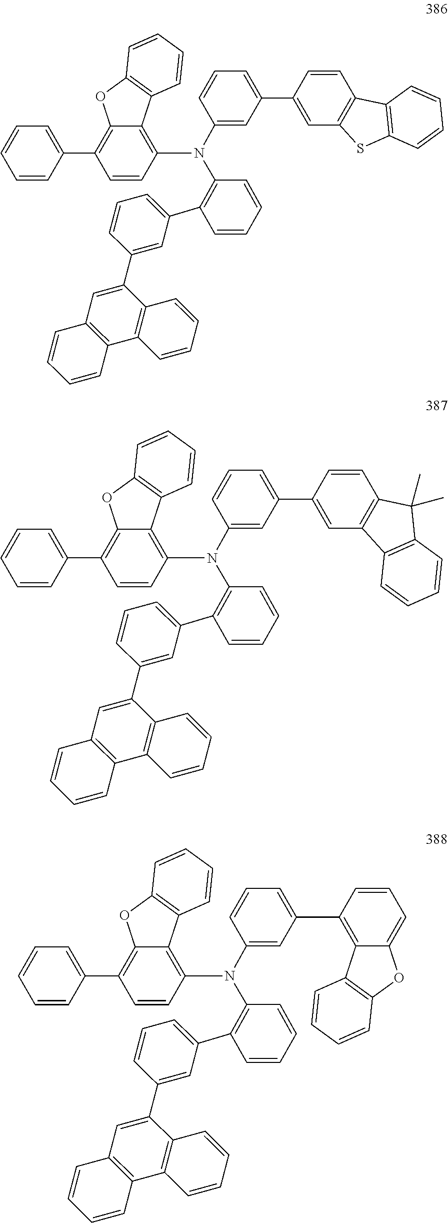

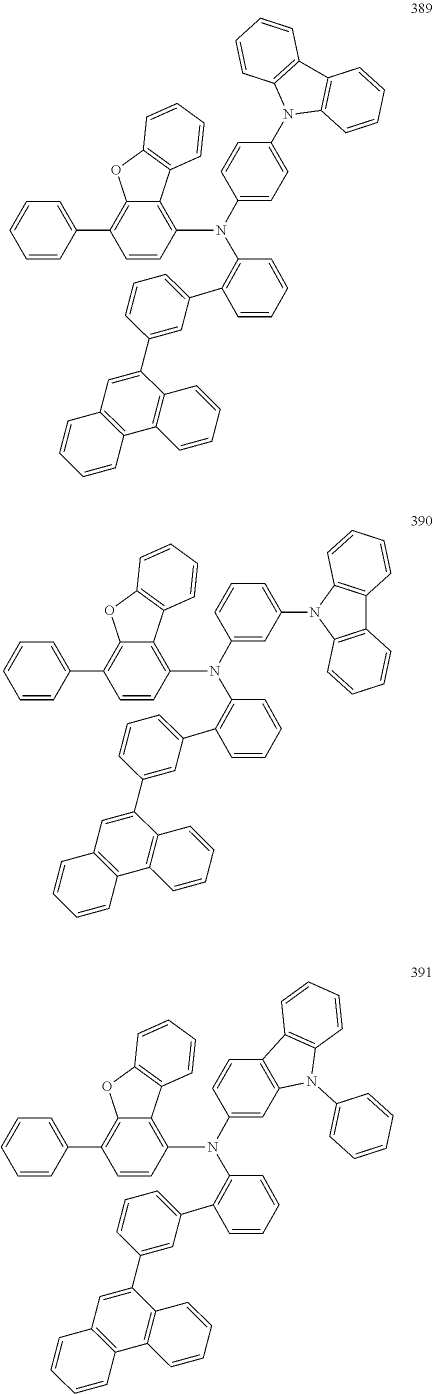

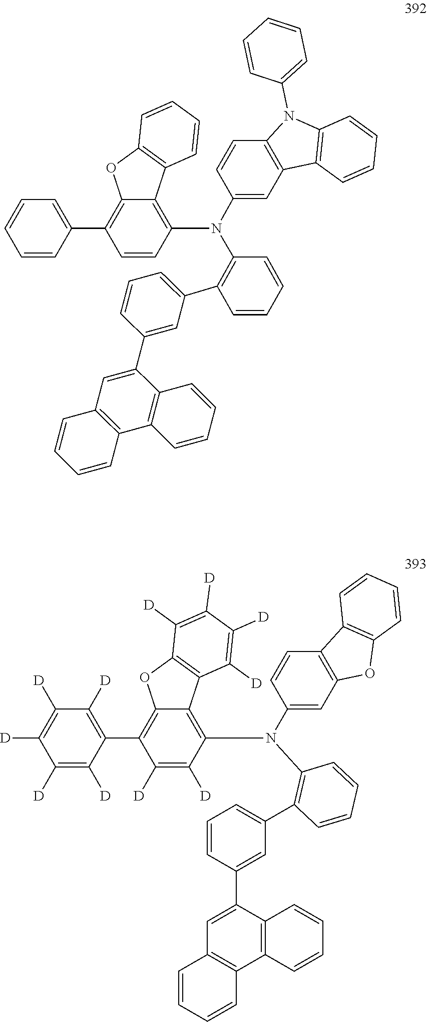 [SMILES: c1ccc(-c2ccc(N(c3ccc4c(c3)c3ccccc3n4-c3ccccc3)c3ccccc3-c3cccc(-c4cc5ccccc5c5ccccc45)c3)c3c2oc2ccccc23)cc1 ; [2H]c1c([2H])c([2H])c(-c2c([2H])c([2H])c(N(c3ccc4c(c3)oc3ccccc34)c3ccccc3-c3cccc(-c4cc5ccccc5c5ccccc45)c3)c3c2oc2c([2H])c([2H])c([2H])c([2H])c23)c([2H])c1[2H]]

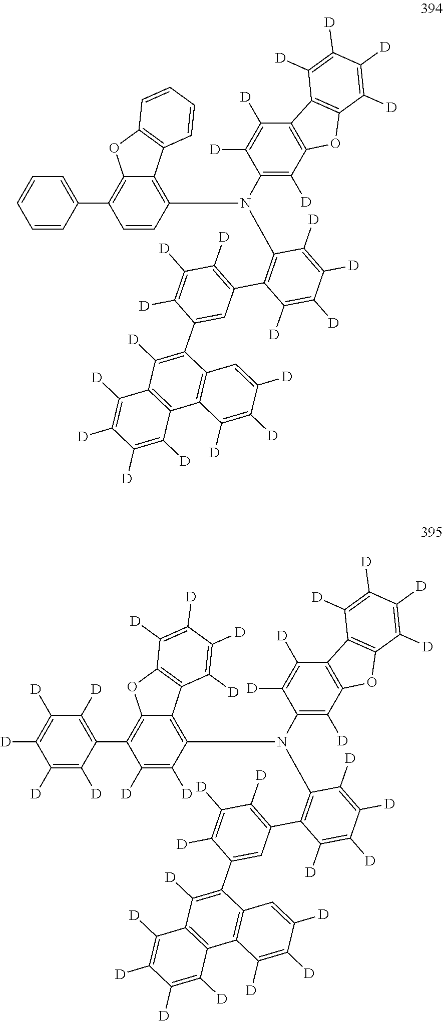

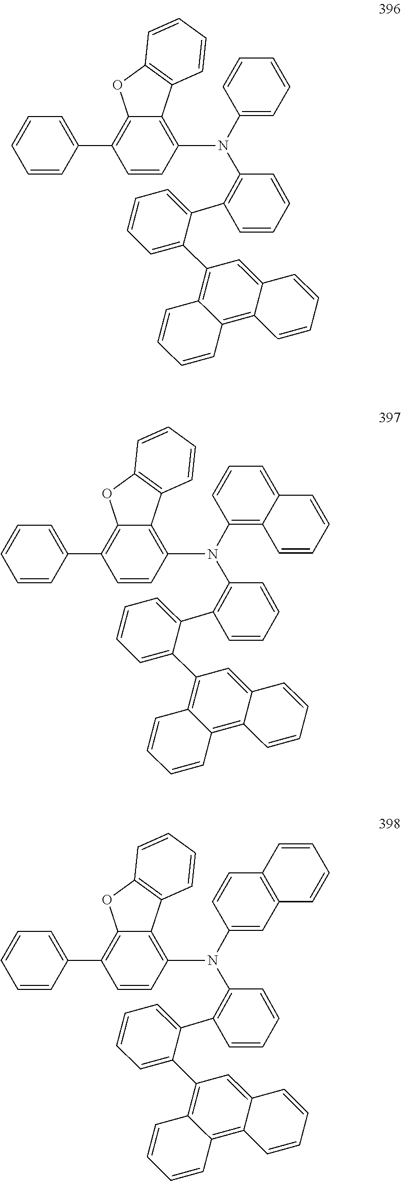 [SMILES: c1ccc(-c2ccc(N(c3ccccc3)c3ccccc3-c3ccccc3-c3cc4ccccc4c4ccccc34)c3c2oc2ccccc23)cc1 ; c1ccc(-c2ccc(N(c3ccccc3-c3ccccc3-c3cc4ccccc4c4ccccc34)c3cccc4ccccc34)c3c2oc2ccccc23)cc1 ; c1ccc(-c2ccc(N(c3ccc4ccccc4c3)c3ccccc3-c3ccccc3-c3cc4ccccc4c4ccccc34)c3c2oc2ccccc23)cc1]

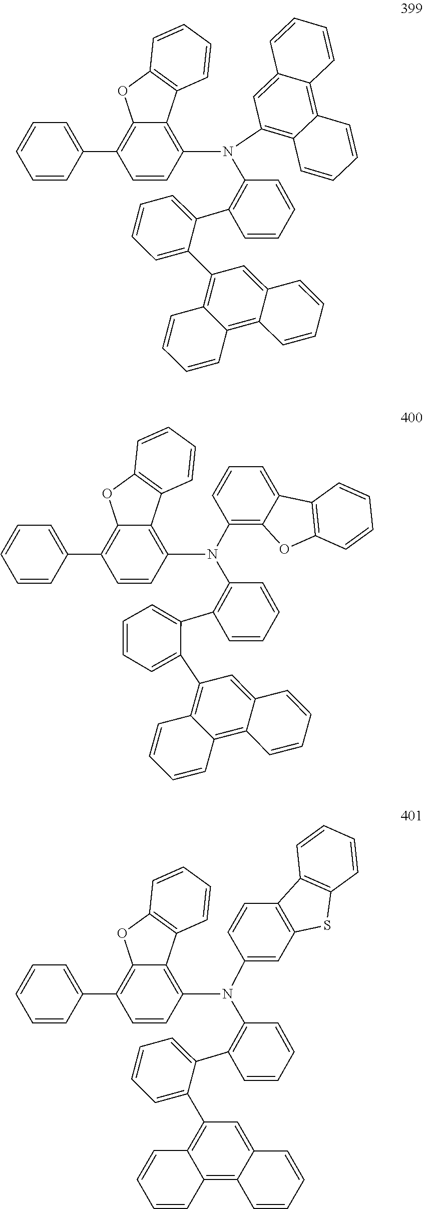 [SMILES: c1ccc(-c2ccc(N(c3ccccc3-c3ccccc3-c3cc4ccccc4c4ccccc34)c3cc4ccccc4c4ccccc34)c3c2oc2ccccc23)cc1 ; c1ccc(-c2ccc(N(c3ccccc3-c3ccccc3-c3cc4ccccc4c4ccccc34)c3cccc4c3oc3ccccc34)c3c2oc2ccccc23)cc1 ; c1ccc(-c2ccc(N(c3ccc4c(c3)sc3ccccc34)c3ccccc3-c3ccccc3-c3cc4ccccc4c4ccccc34)c3c2oc2ccccc23)cc1]

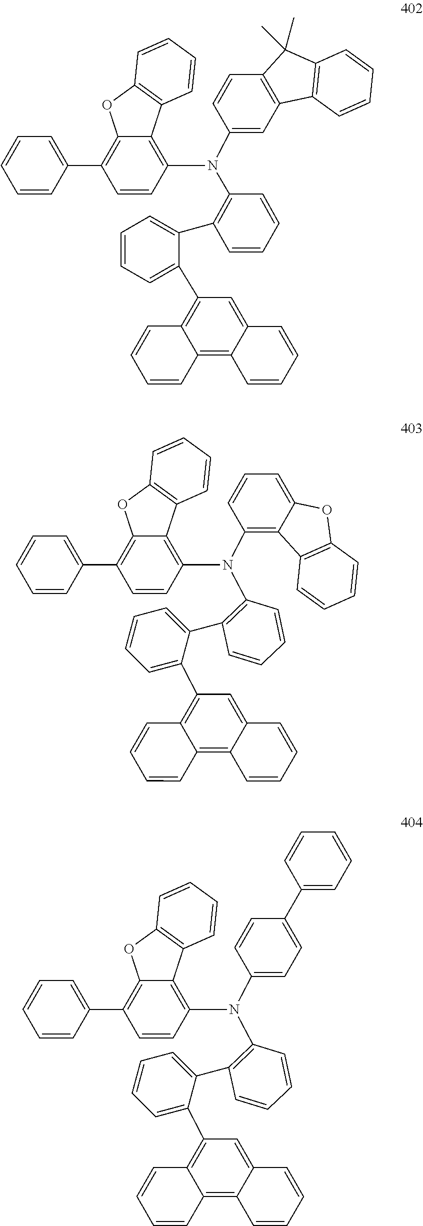 [SMILES: CC1(C)c2ccccc2-c2cc(N(c3ccccc3-c3ccccc3-c3cc4ccccc4c4ccccc34)c3ccc(-c4ccccc4)c4oc5ccccc5c34)ccc21 ; c1ccc(-c2ccc(N(c3ccccc3-c3ccccc3-c3cc4ccccc4c4ccccc34)c3cccc4oc5ccccc5c34)c3c2oc2ccccc23)cc1 ; c1ccc(-c2ccc(N(c3ccccc3-c3ccccc3-c3cc4ccccc4c4ccccc34)c3ccc(-c4ccccc4)c4oc5ccccc5c34)cc2)cc1]

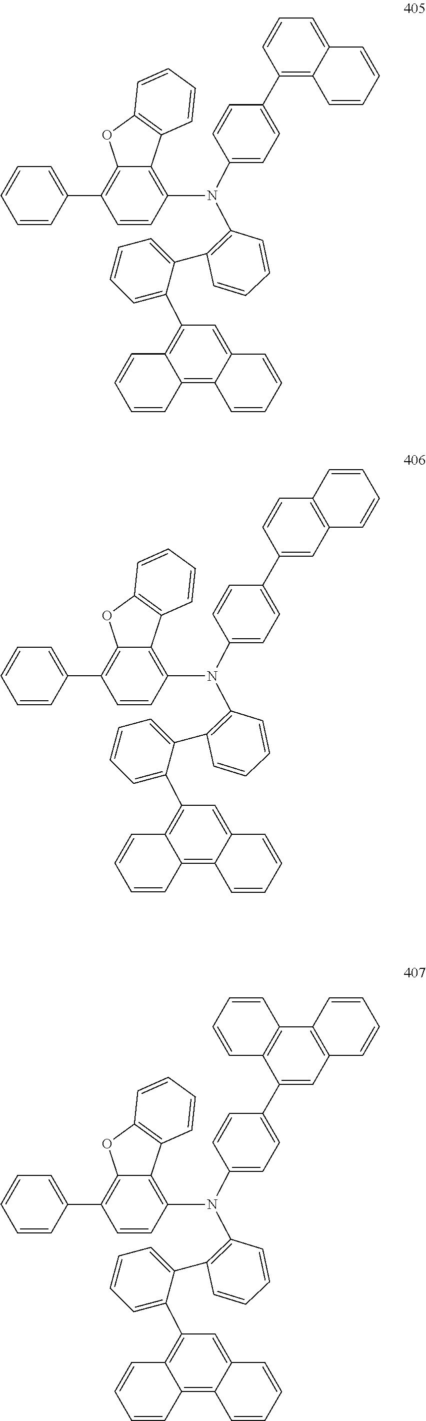 [SMILES: c1ccc(-c2ccc(N(c3ccc(-c4cccc5ccccc45)cc3)c3ccccc3-c3ccccc3-c3cc4ccccc4c4ccccc34)c3c2oc2ccccc23)cc1 ; c1ccc(-c2ccc(N(c3ccc(-c4ccc5ccccc5c4)cc3)c3ccccc3-c3ccccc3-c3cc4ccccc4c4ccccc34)c3c2oc2ccccc23)cc1 ; c1ccc(-c2ccc(N(c3ccc(-c4cc5ccccc5c5ccccc45)cc3)c3ccccc3-c3ccccc3-c3cc4ccccc4c4ccccc34)c3c2oc2ccccc23)cc1]

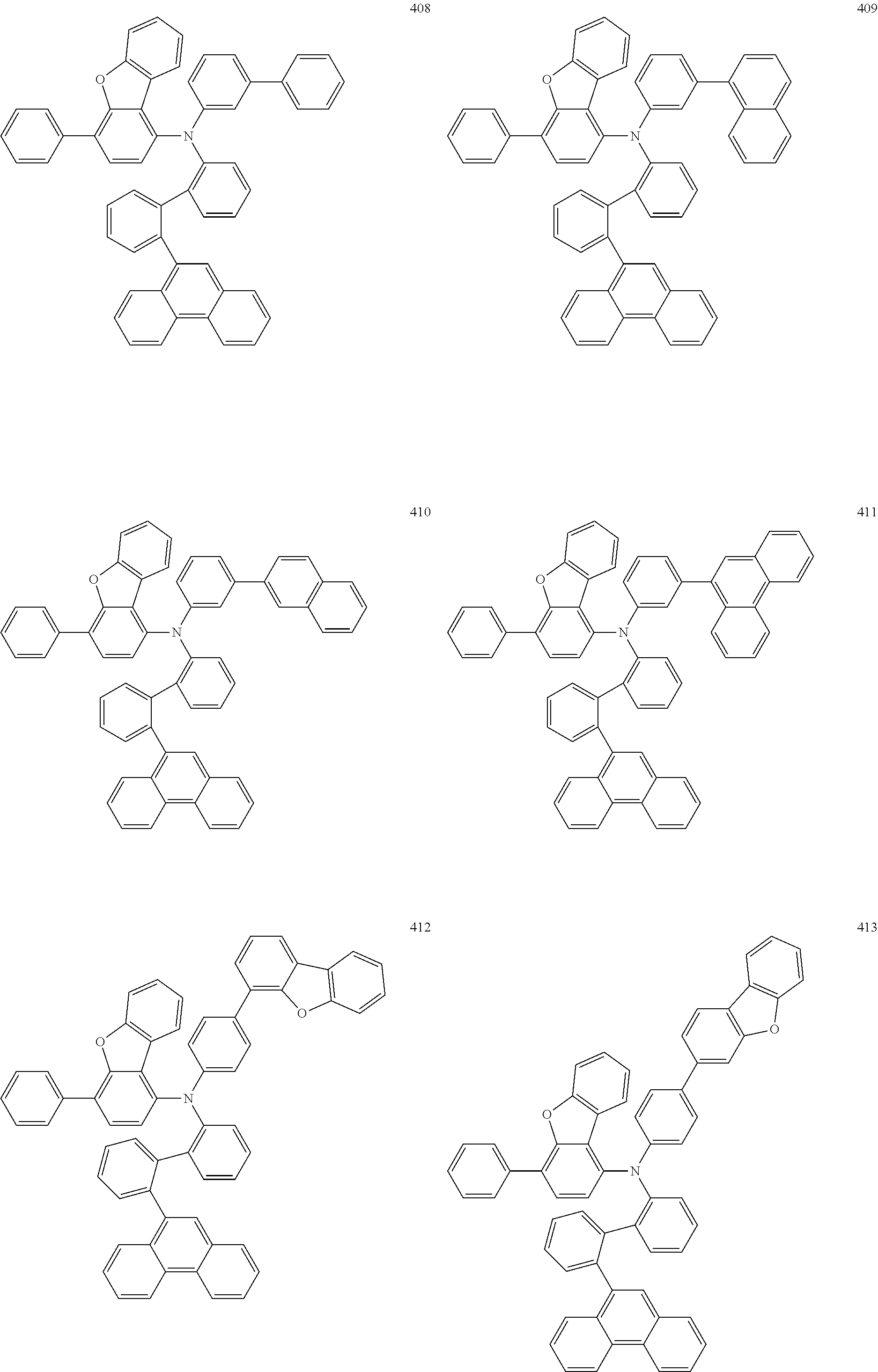

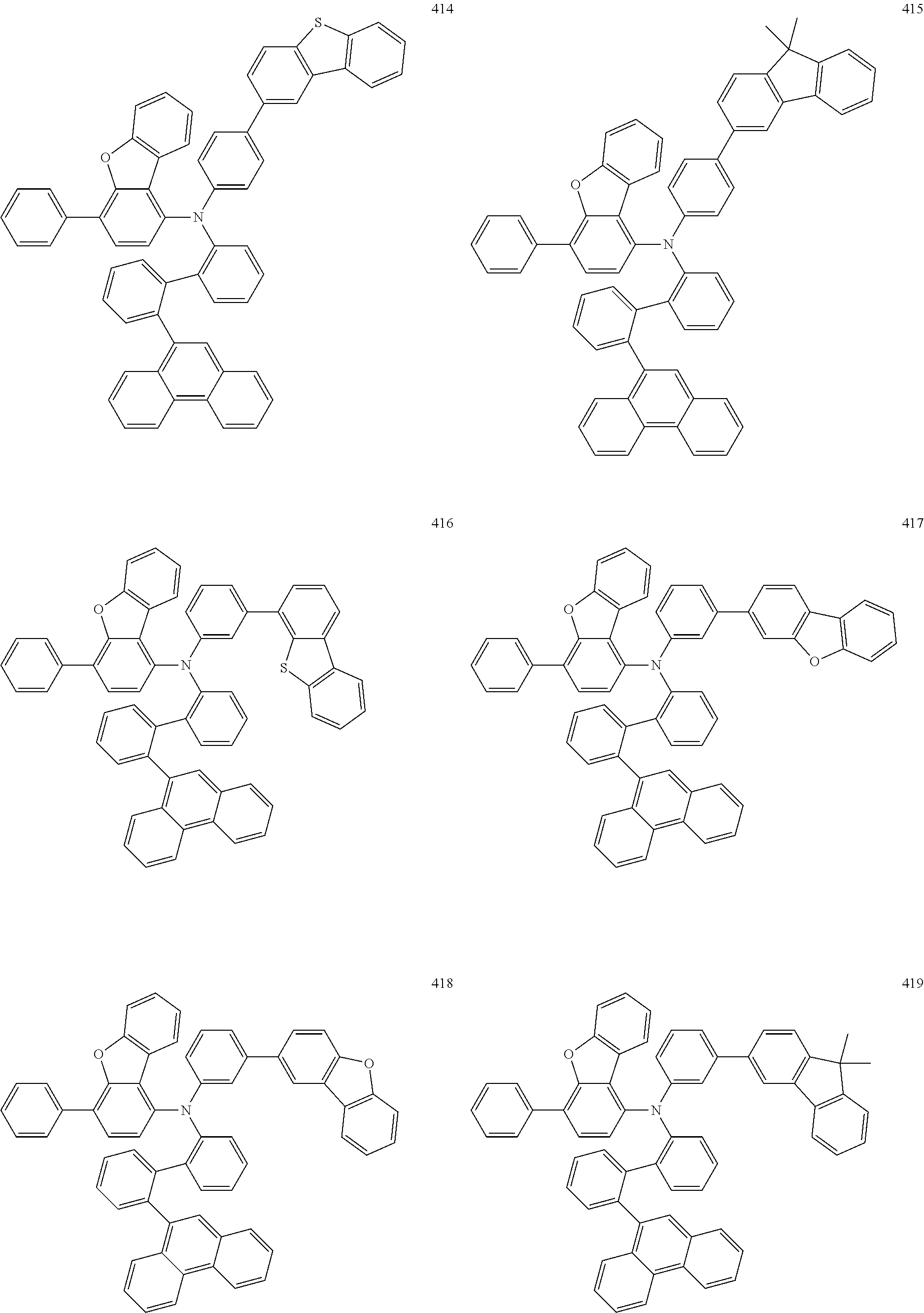

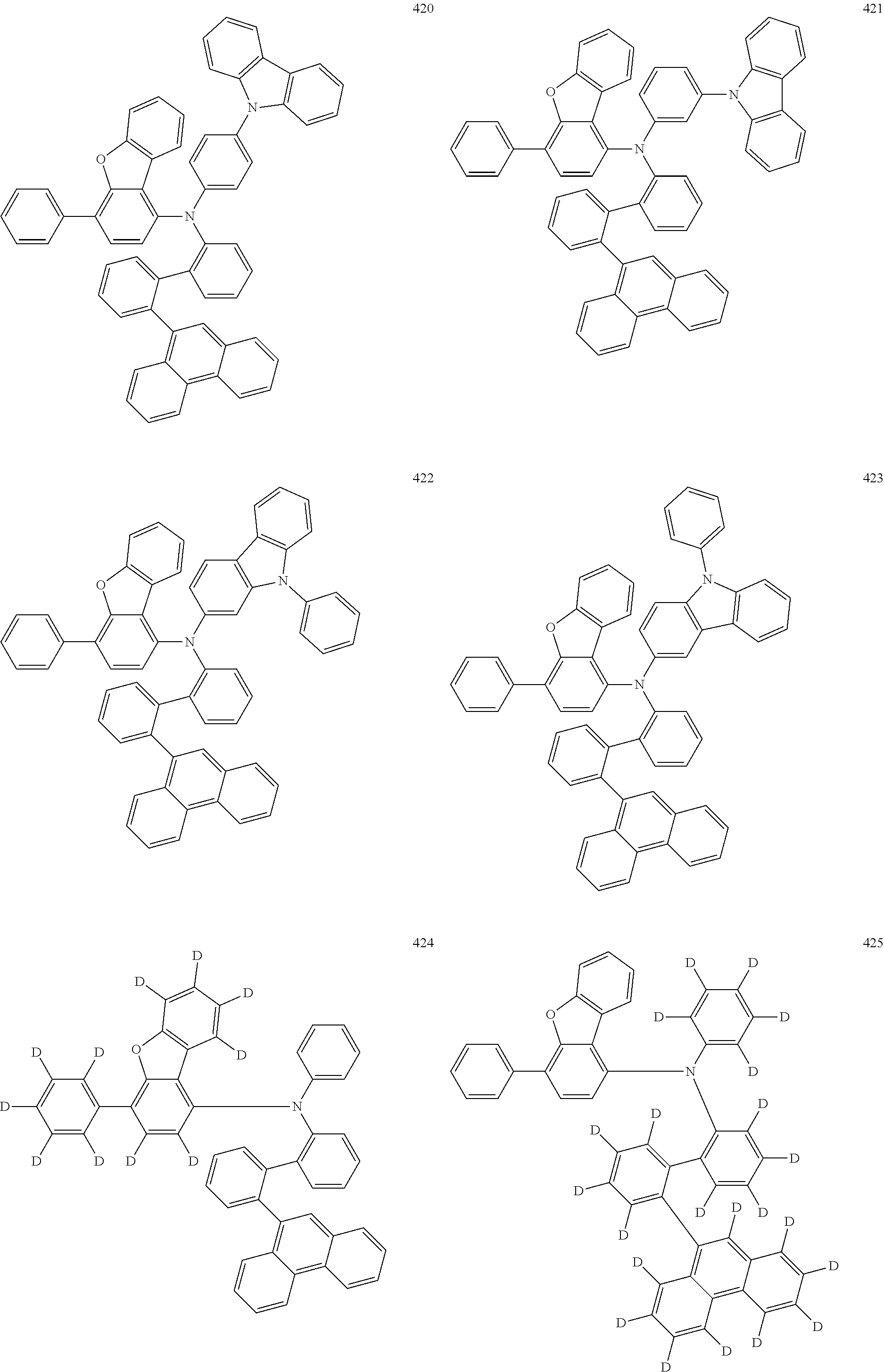

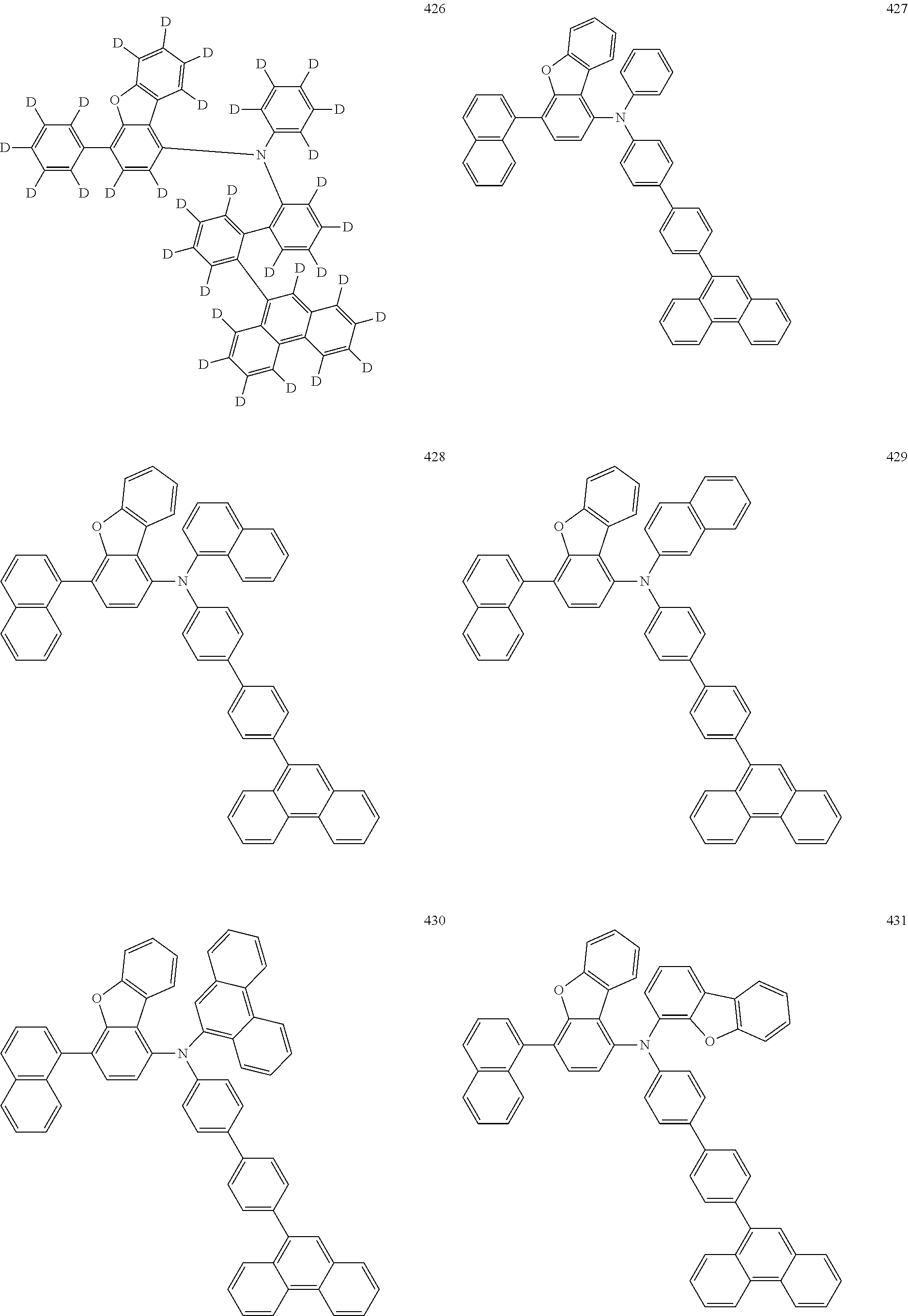

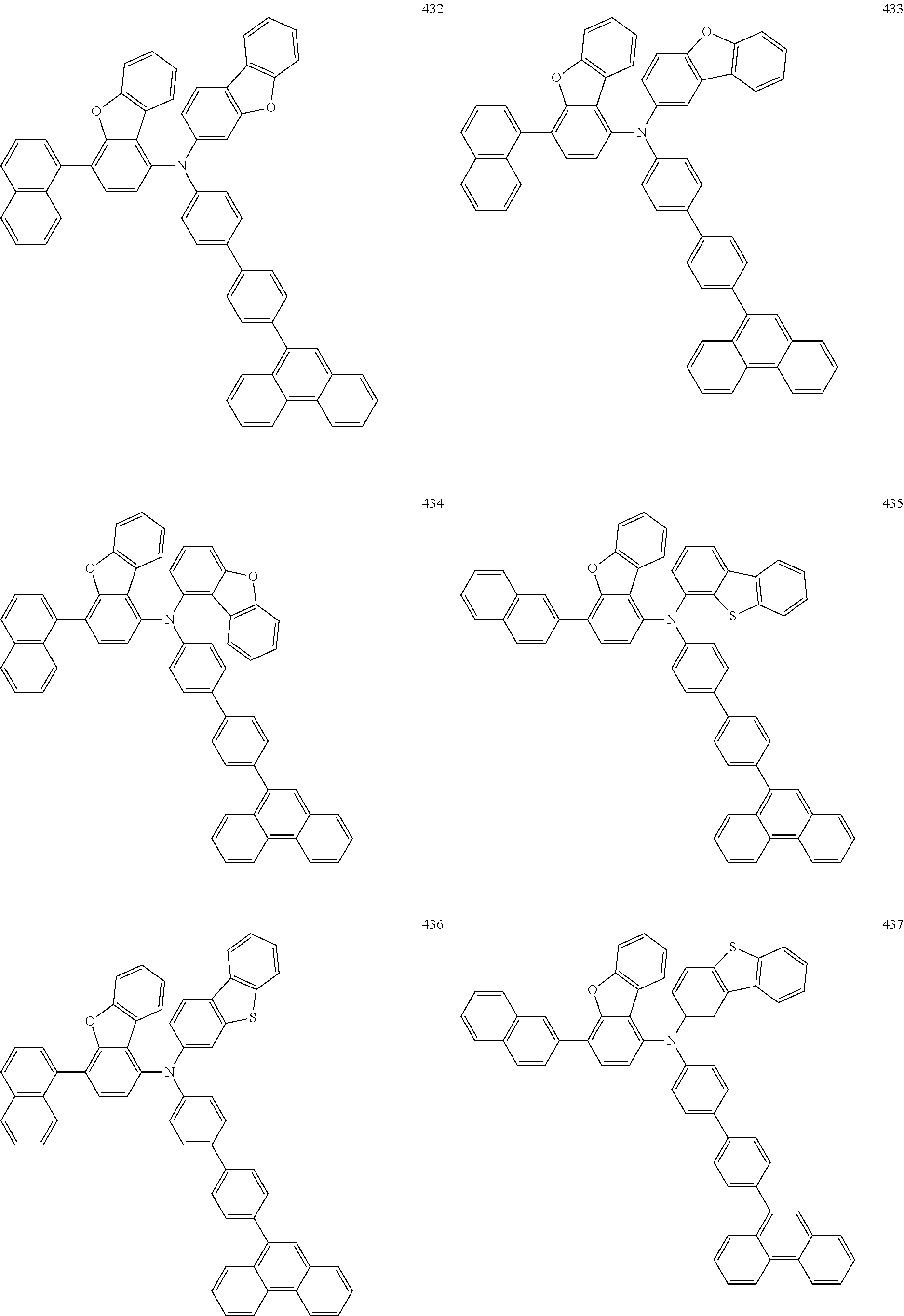

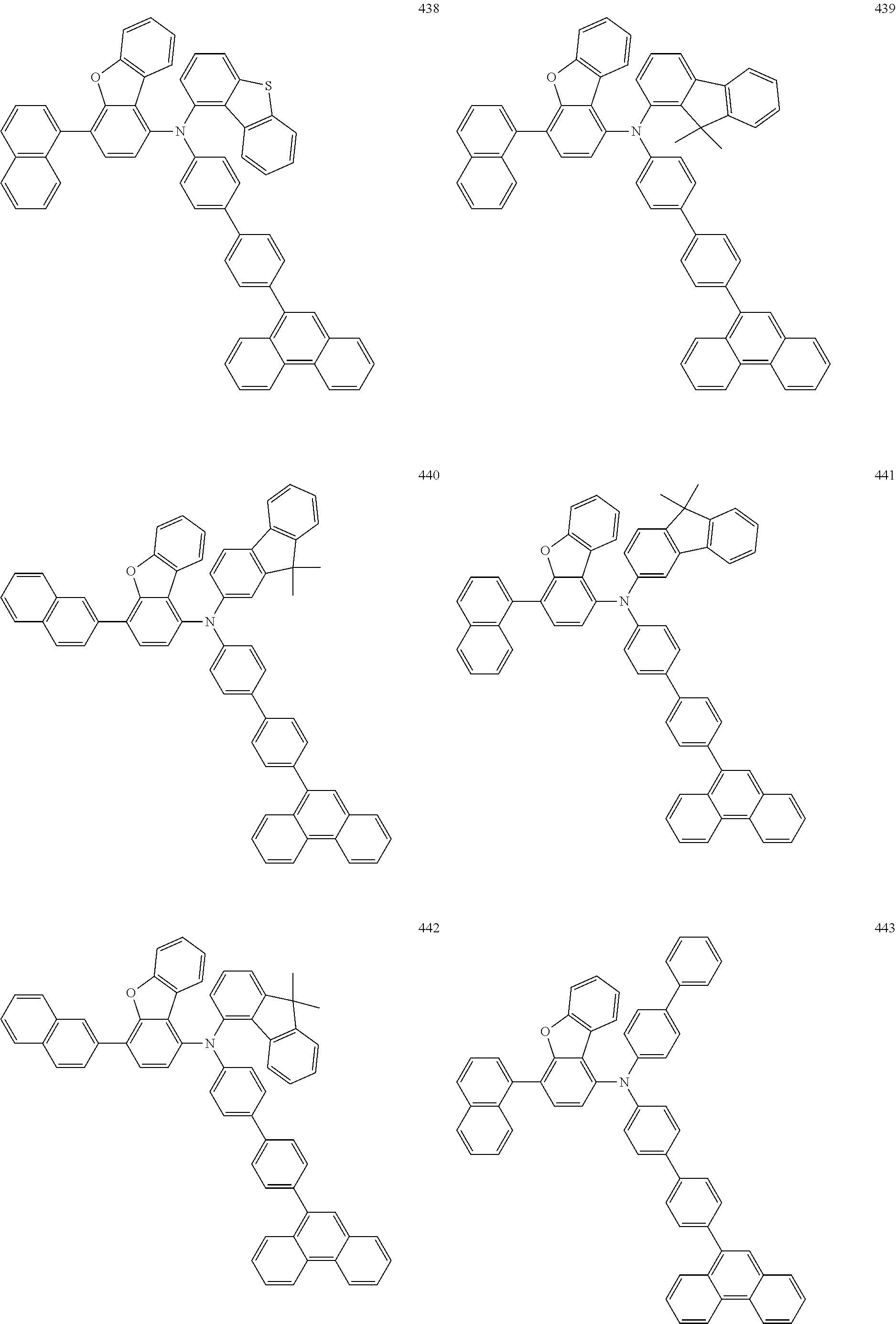

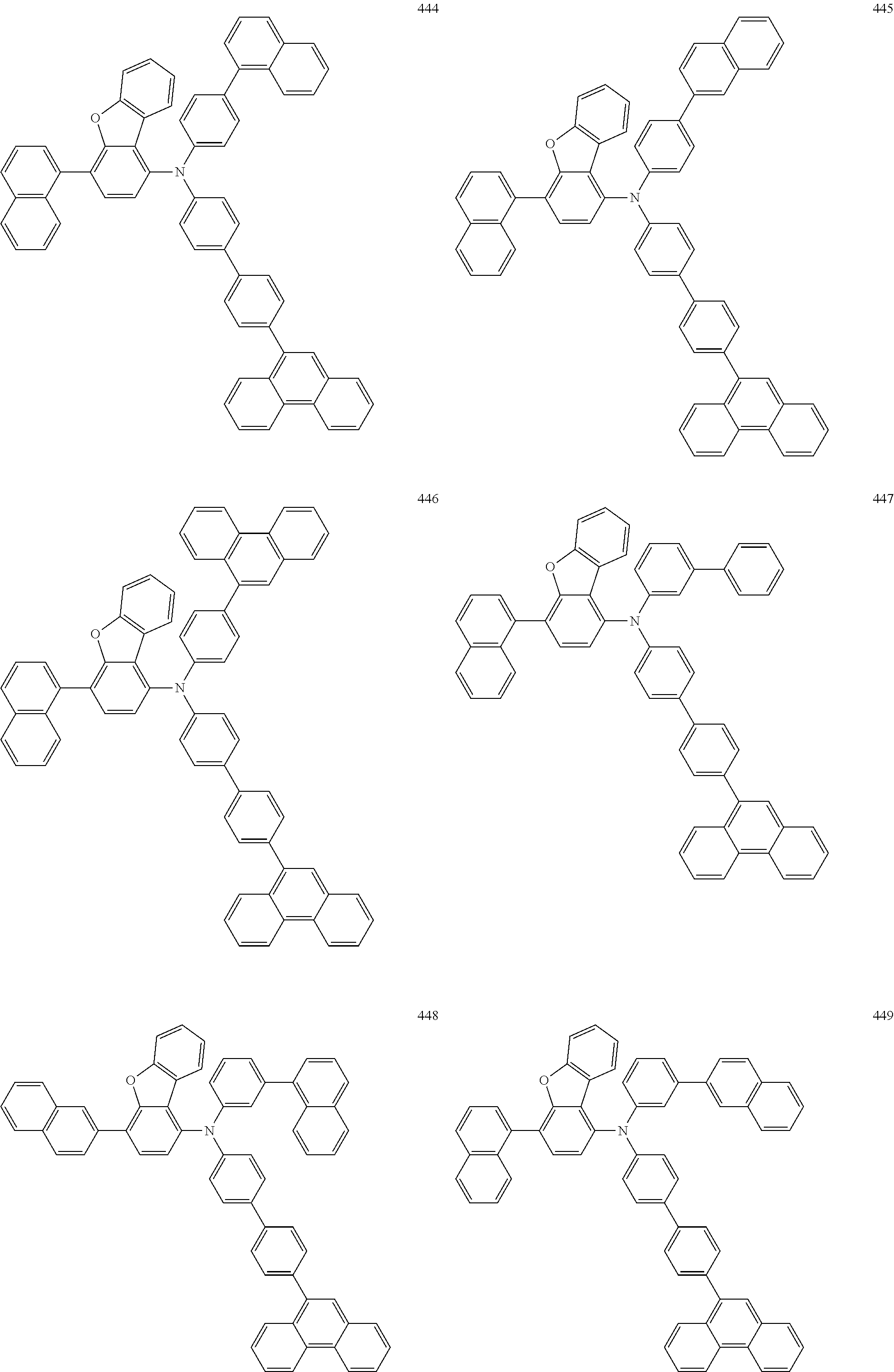

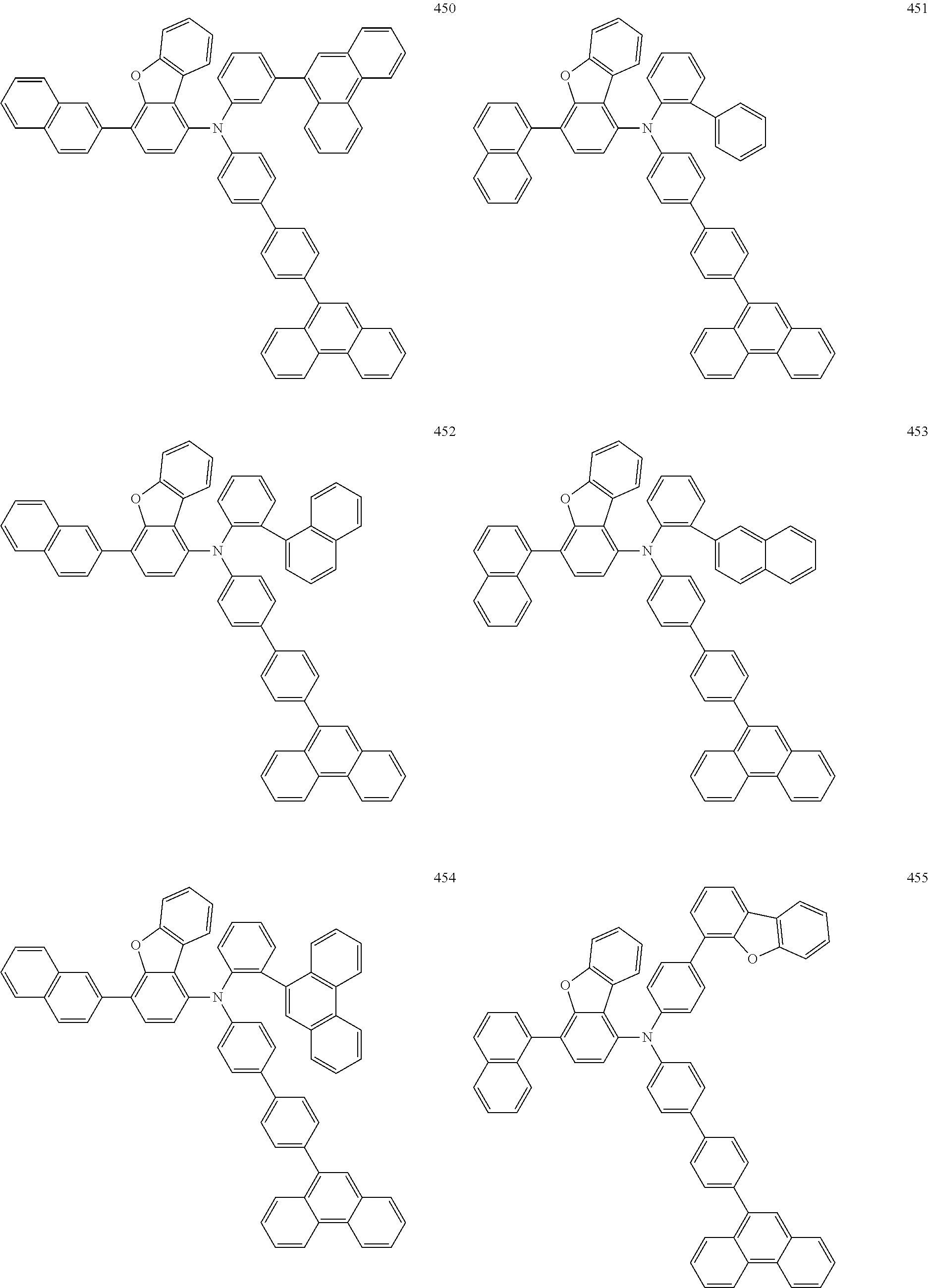

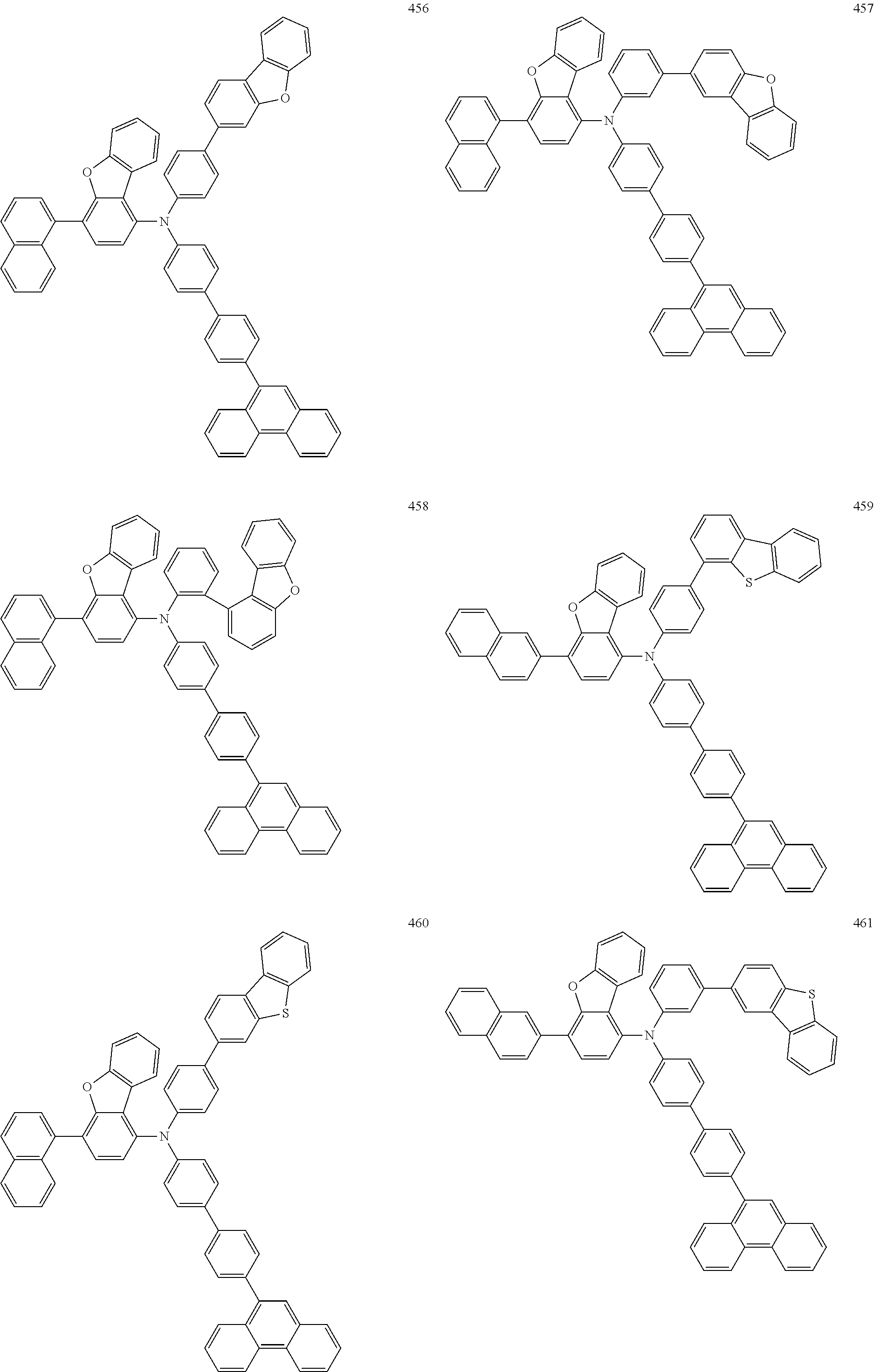

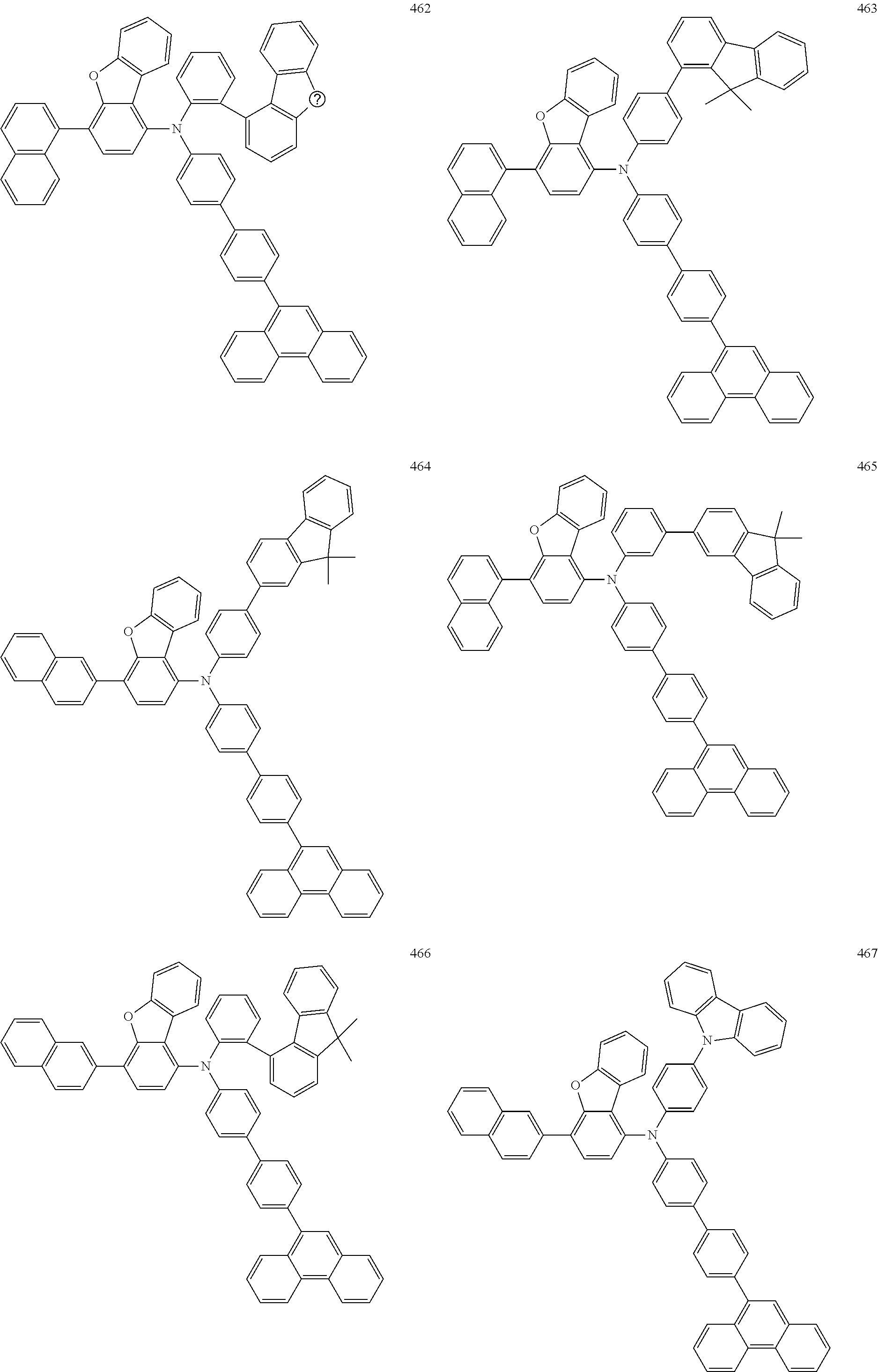

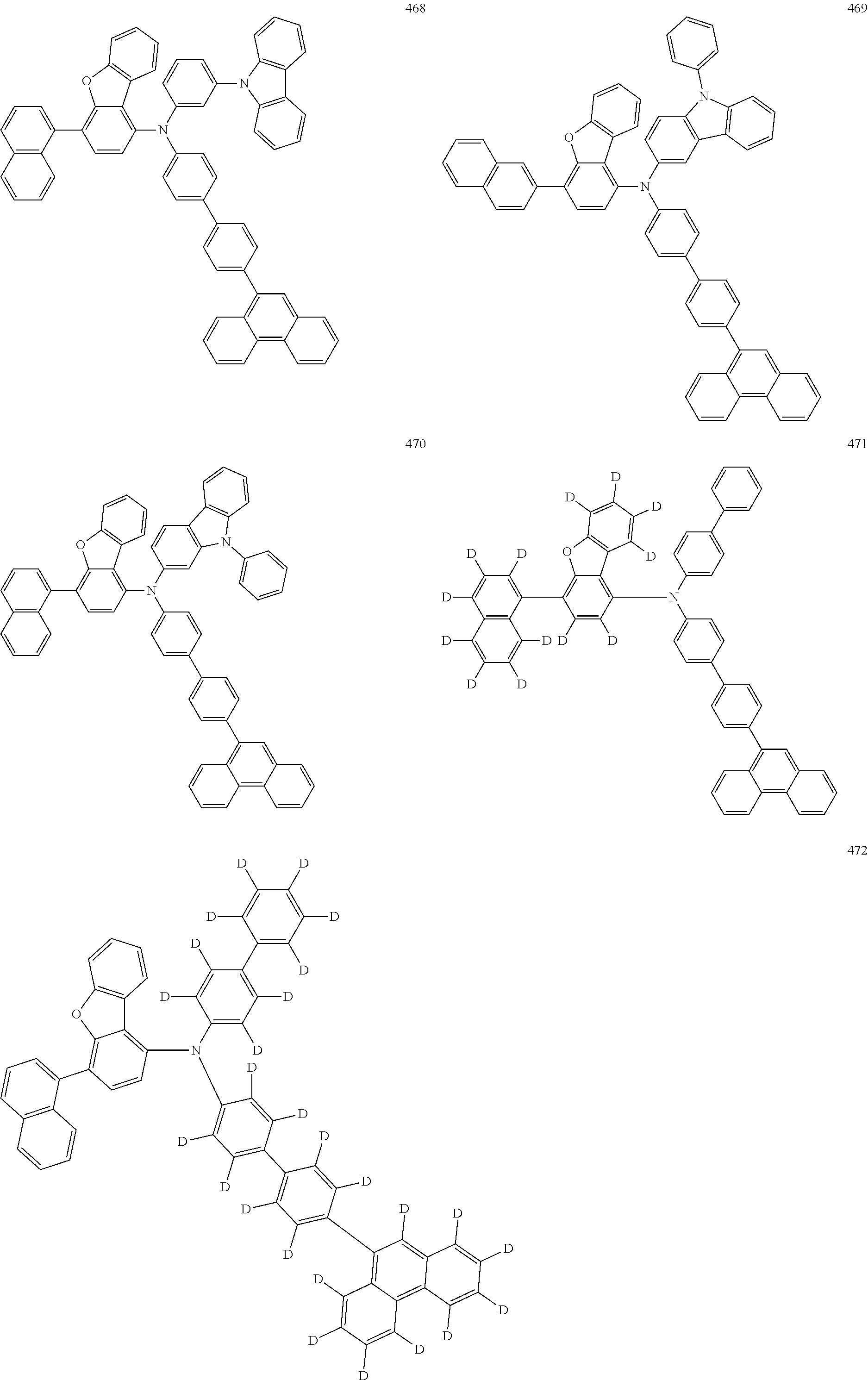

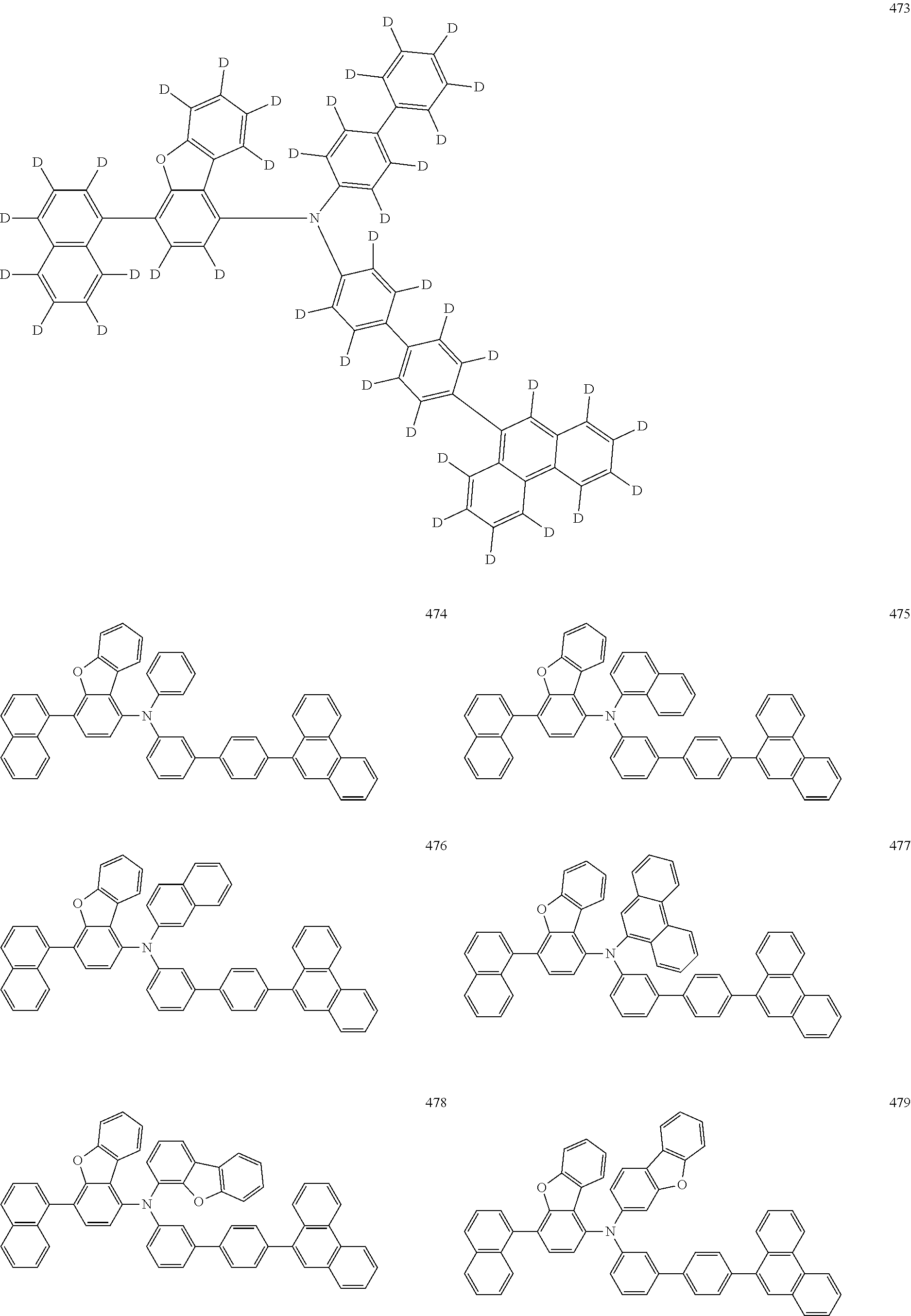

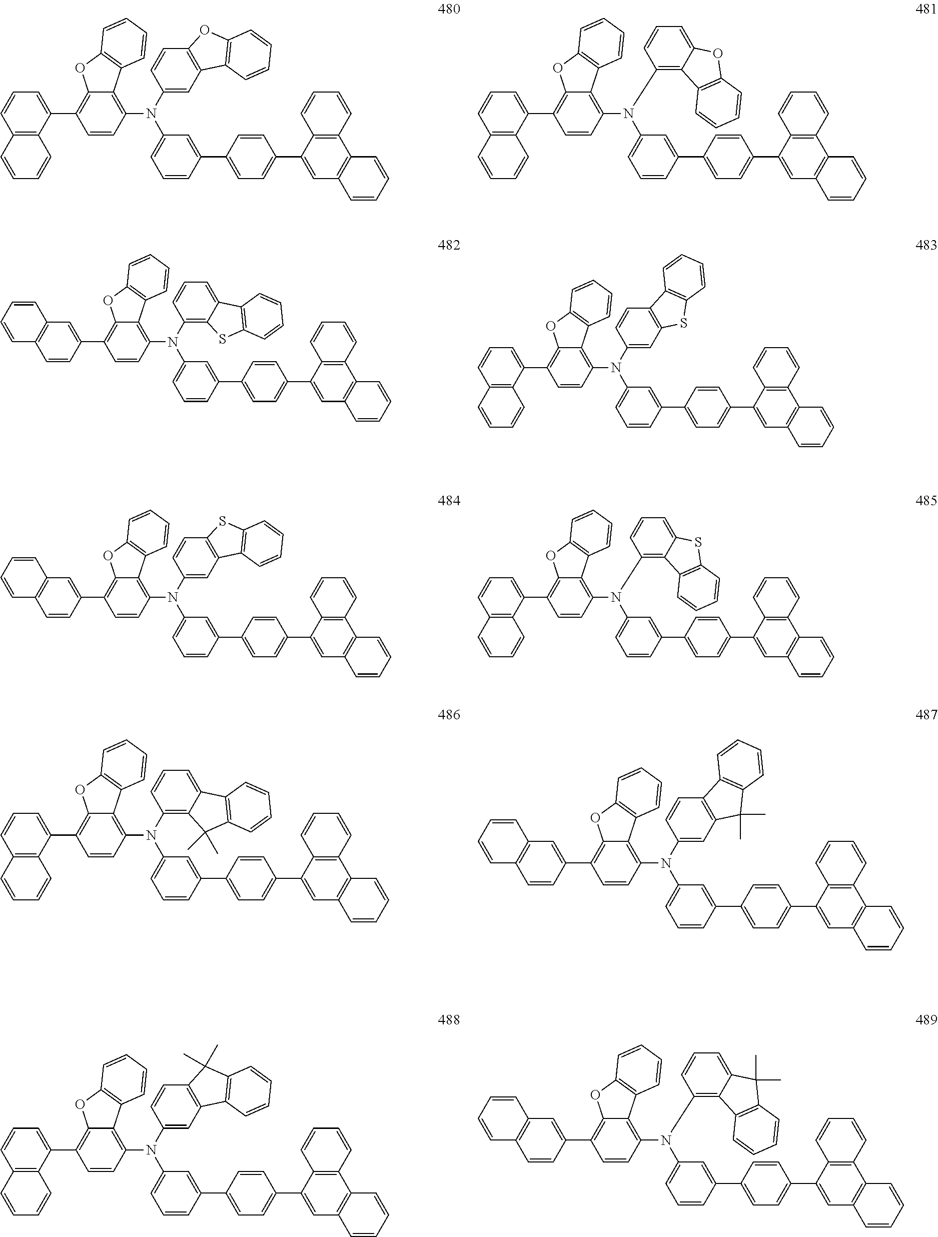

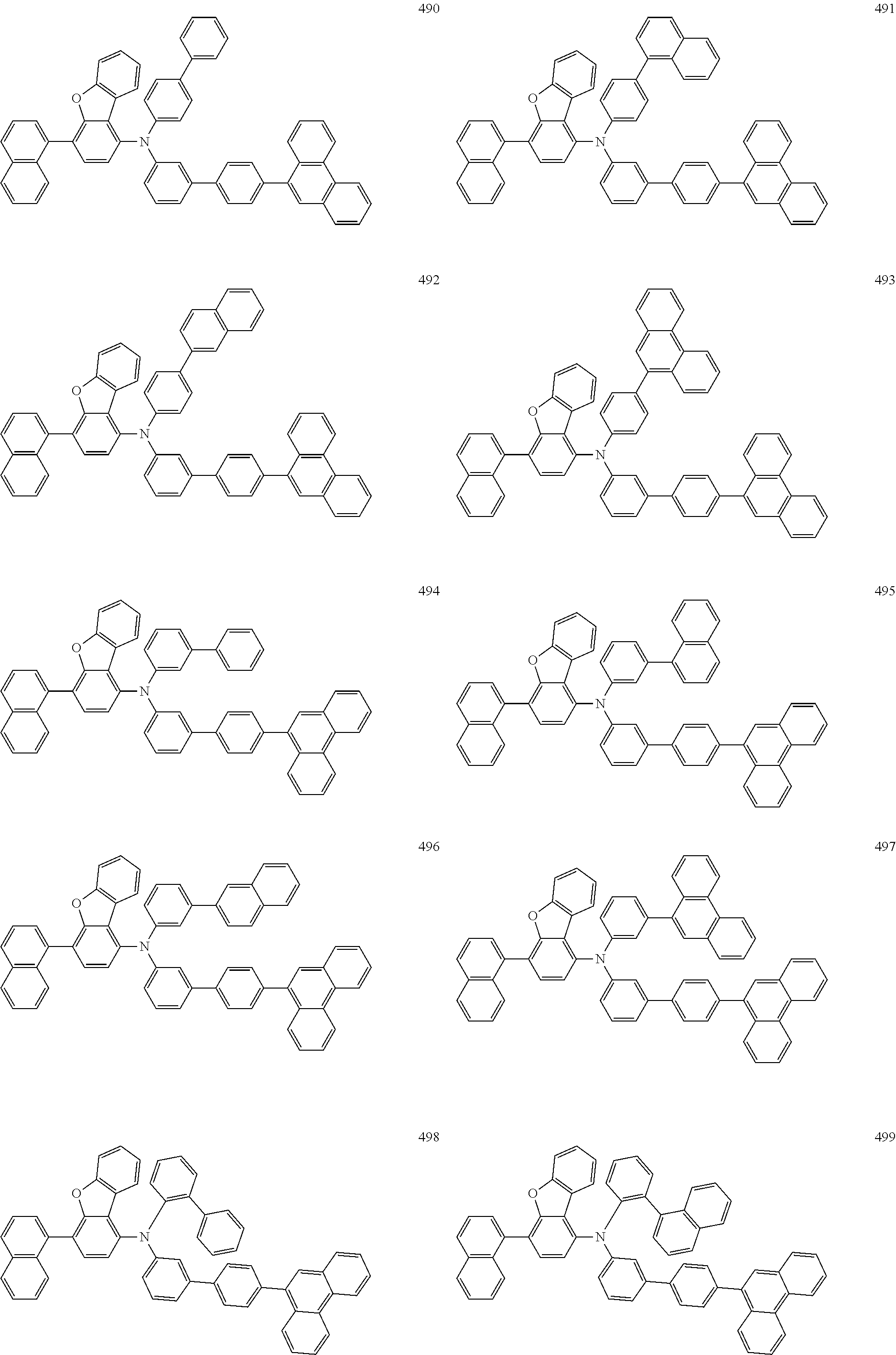

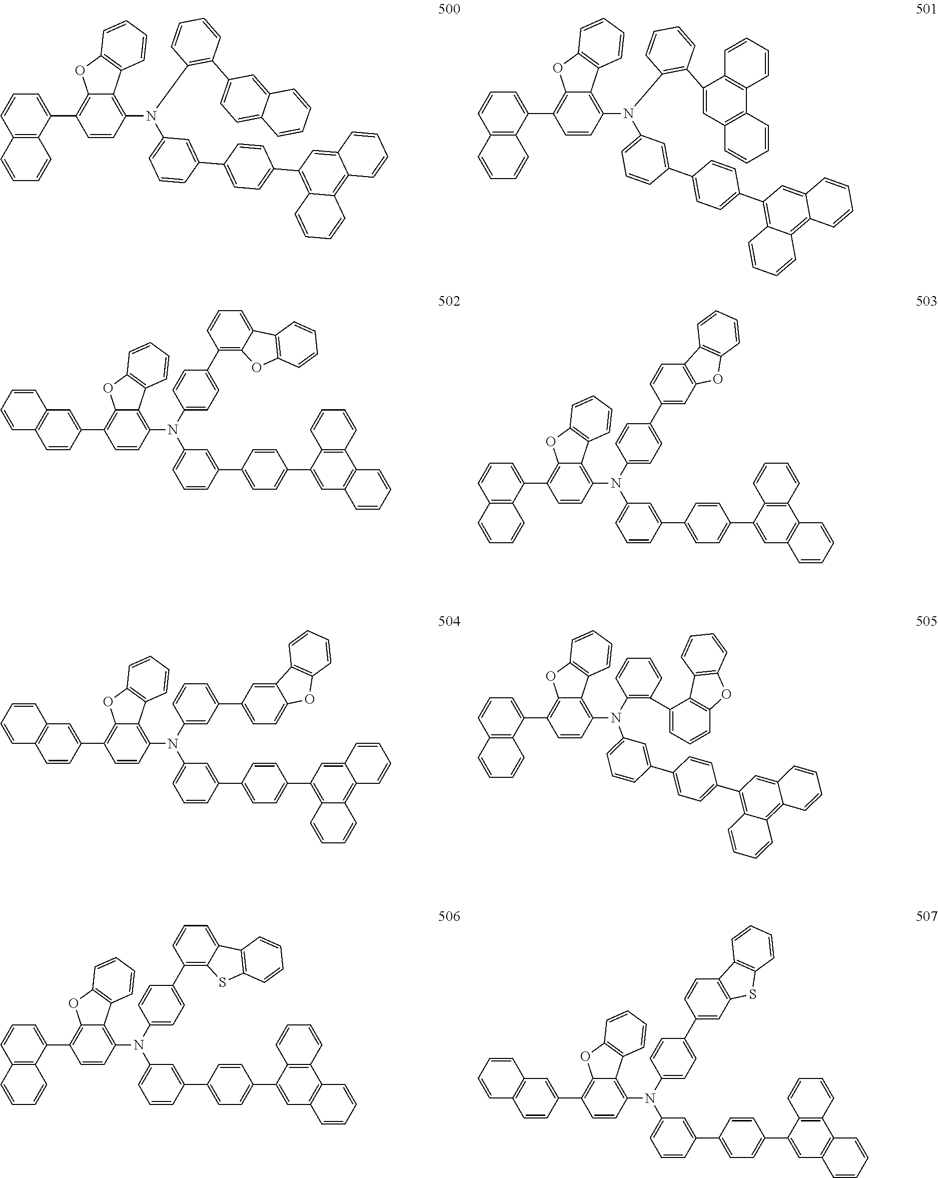

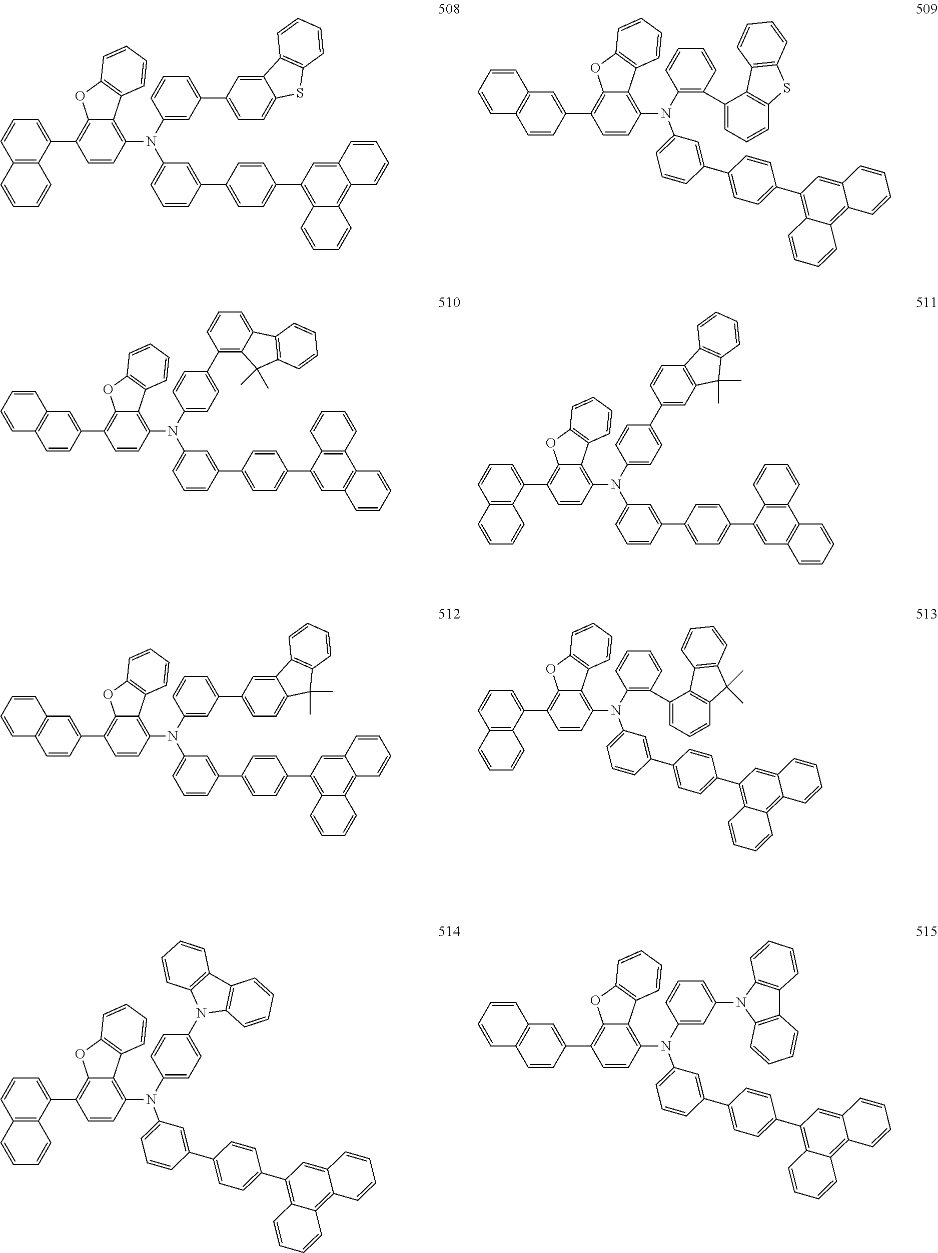

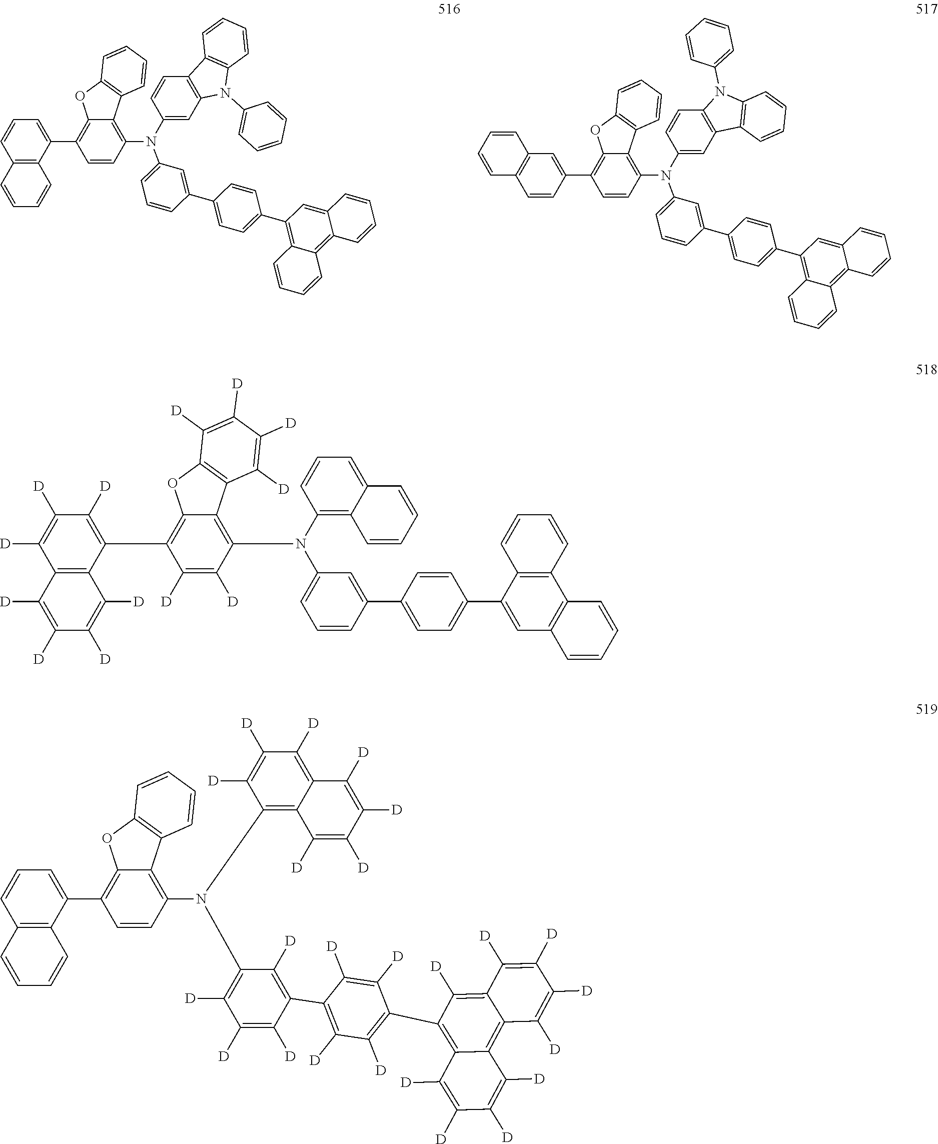

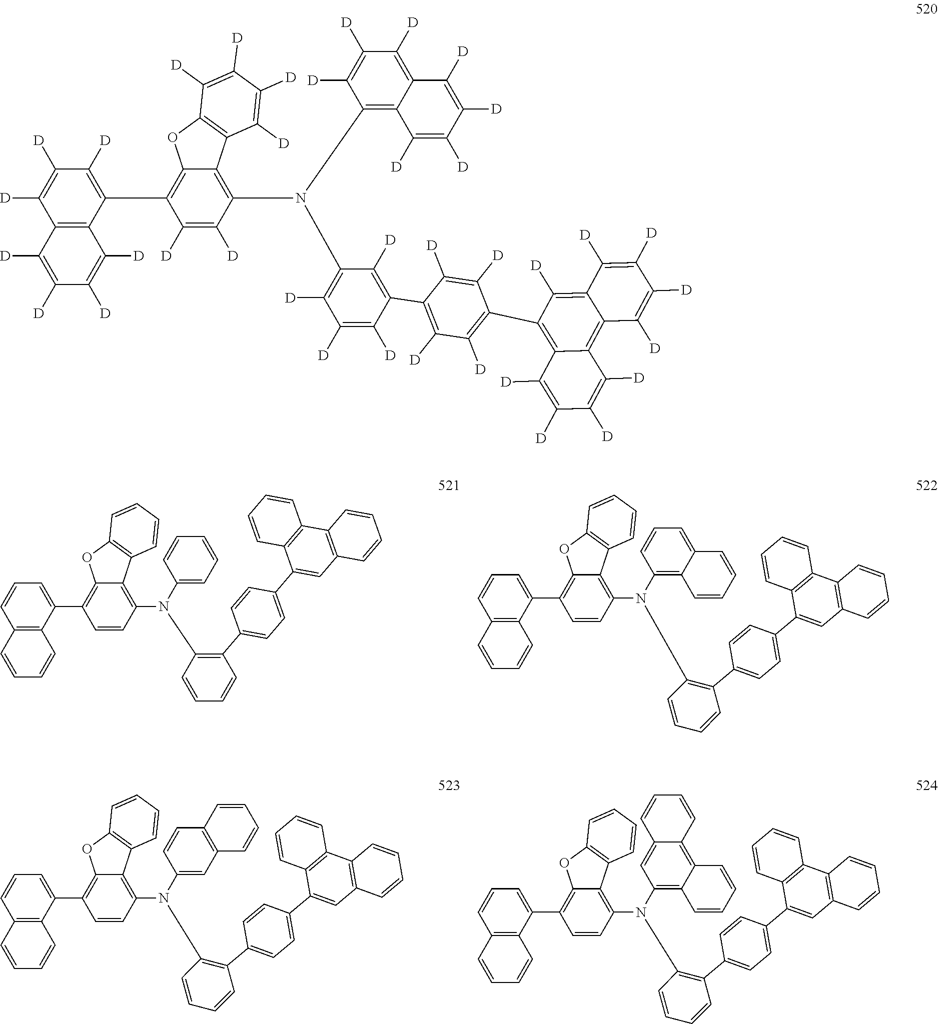

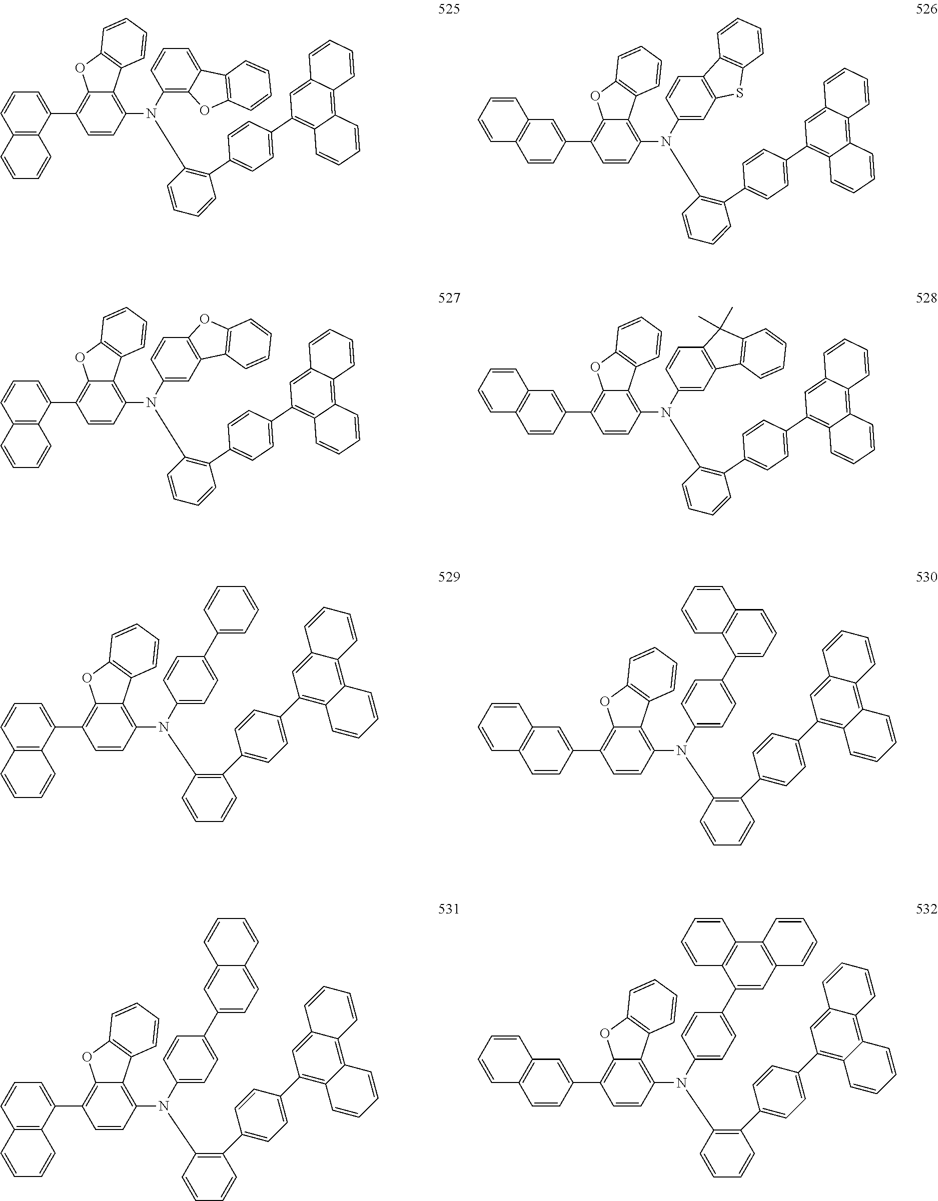

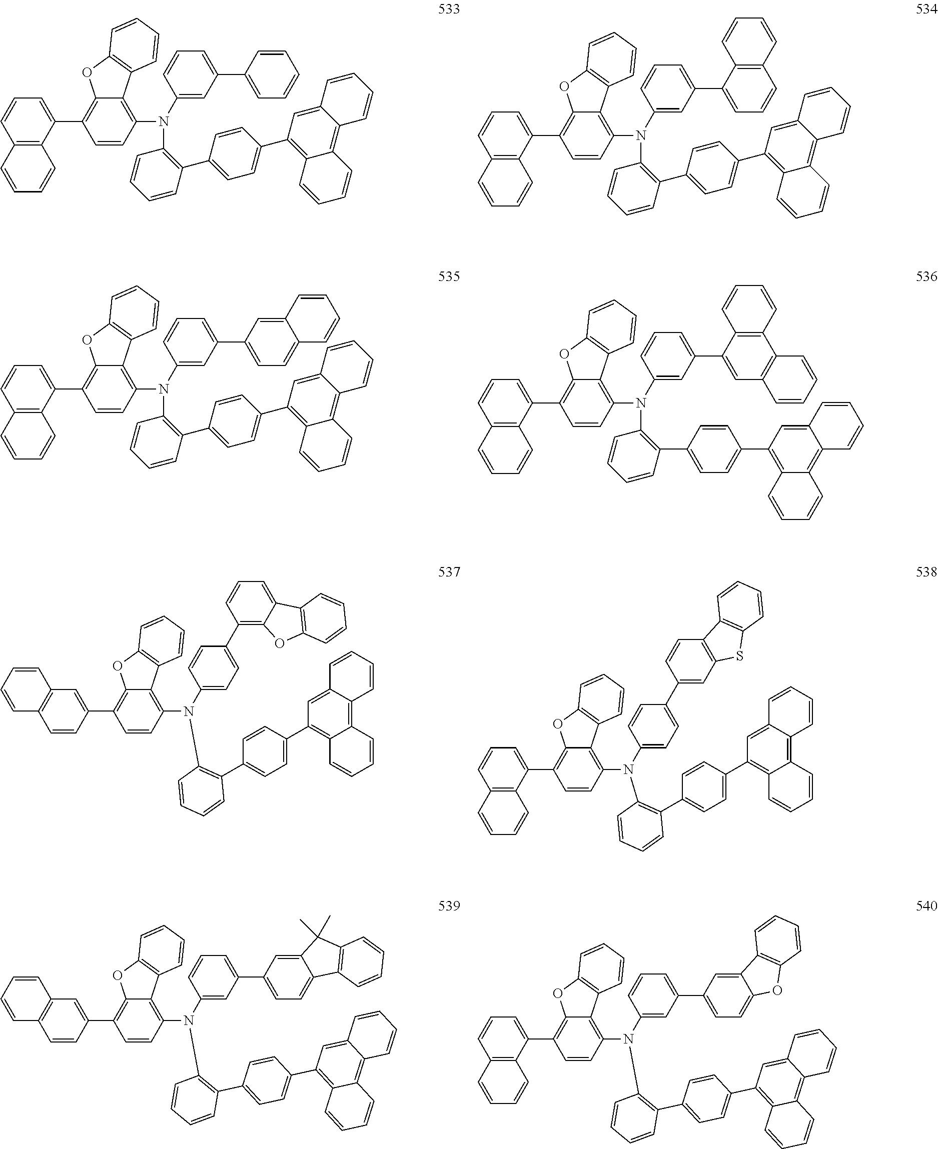

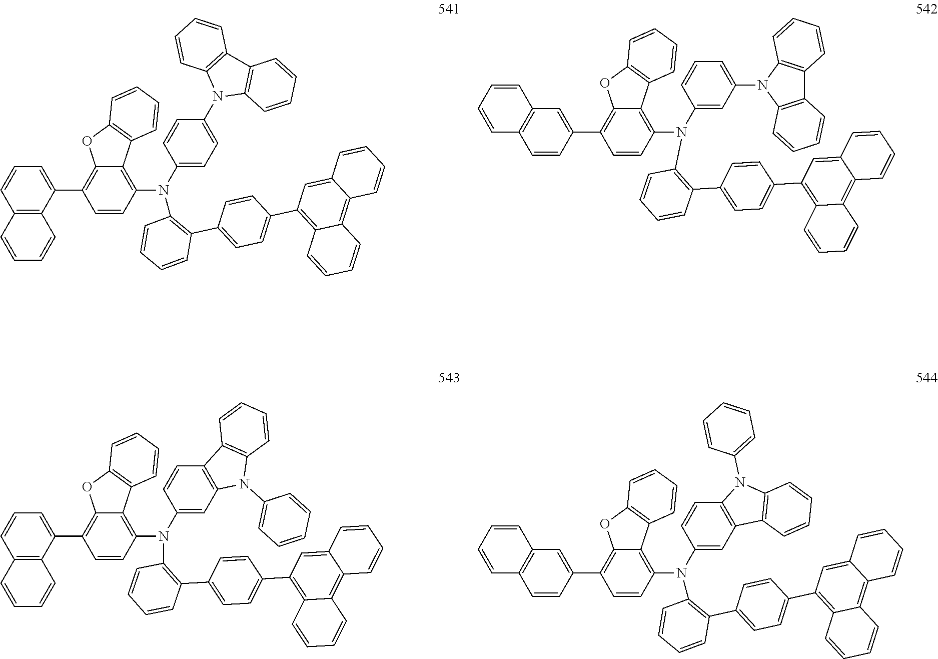 [SMILES: c1ccc(N(c2ccc(-n3c4ccccc4c4ccccc43)cc2)c2ccc(-c3cccc4ccccc34)c3oc4ccccc4c23)c(-c2ccc(-c3cc4ccccc4c4ccccc34)cc2)c1 ; c1cc(N(c2ccccc2-c2ccc(-c3cc4ccccc4c4ccccc34)cc2)c2ccc(-c3ccc4ccccc4c3)c3oc4ccccc4c23)cc(-n2c3ccccc3c3ccccc32)c1 ; c1ccc(-n2c3ccccc3c3ccc(N(c4ccccc4-c4ccc(-c5cc6ccccc6c6ccccc56)cc4)c4ccc(-c5cccc6ccccc56)c5oc6ccccc6c45)cc32)cc1 ; c1ccc(-n2c3ccccc3c3cc(N(c4ccccc4-c4ccc(-c5cc6ccccc6c6ccccc56)cc4)c4ccc(-c5ccc6ccccc6c5)c5oc6ccccc6c45)ccc32)cc1]

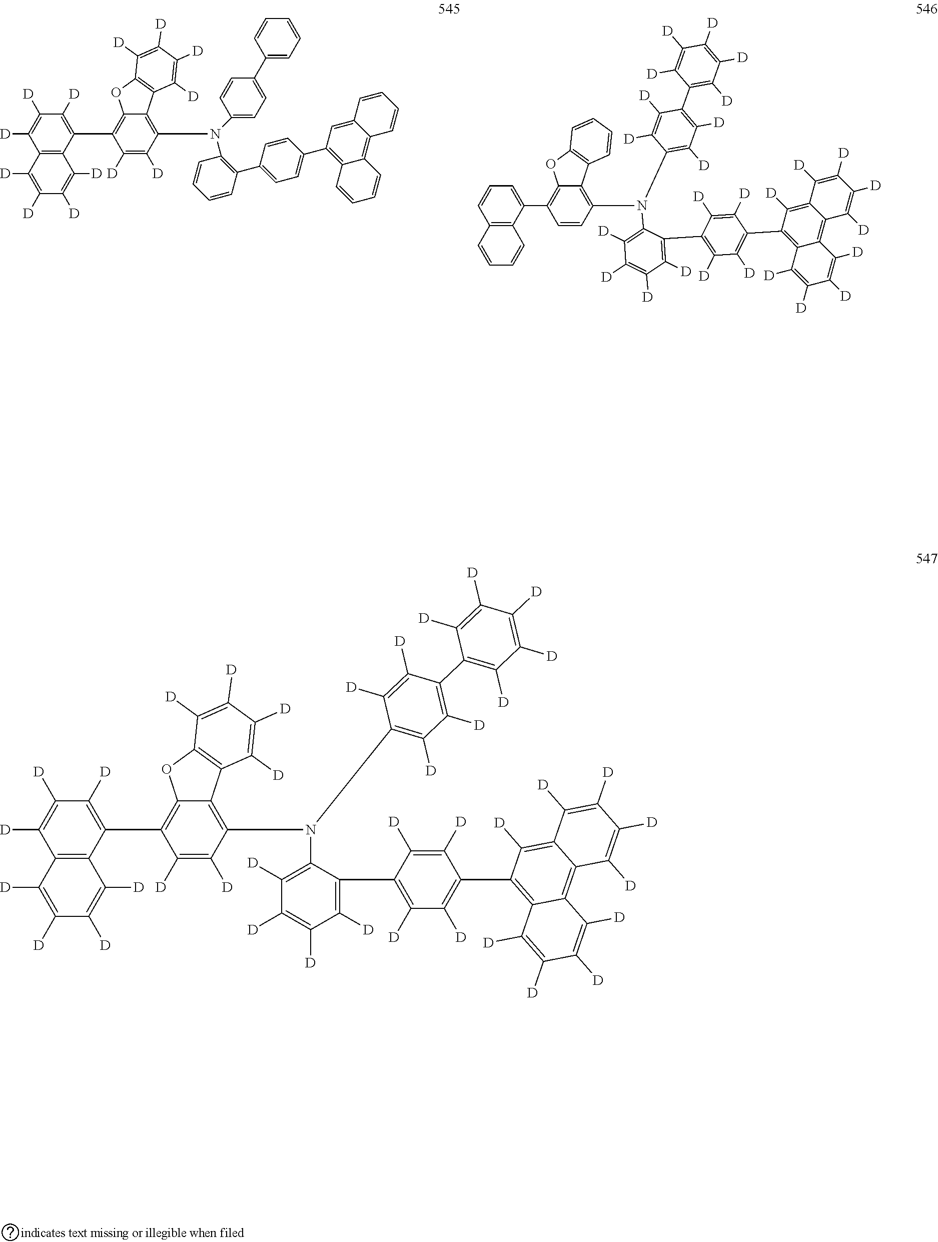

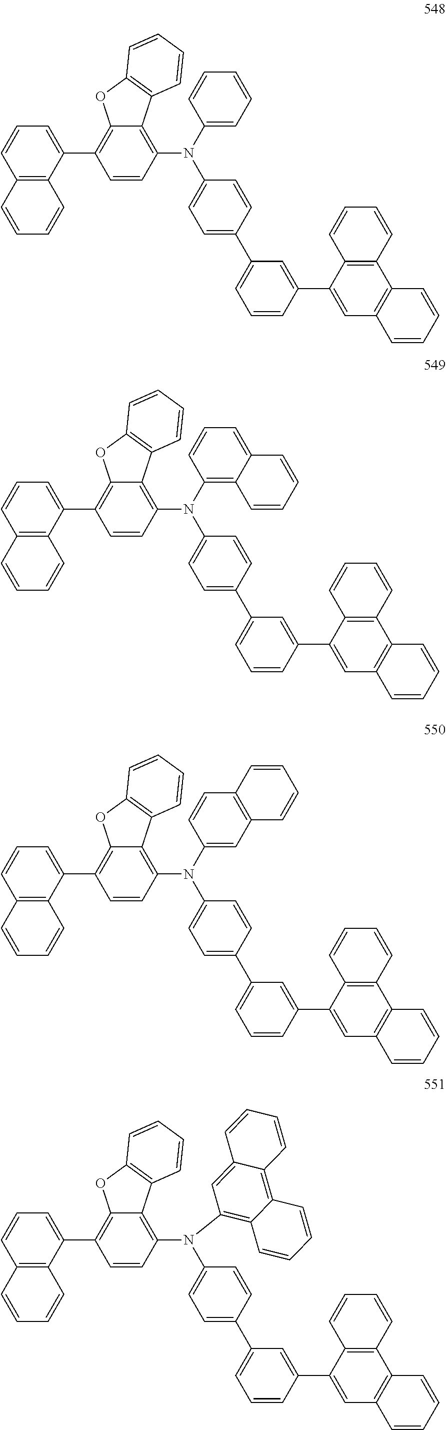 [SMILES: c1ccc(N(c2ccc(-c3cccc(-c4cc5ccccc5c5ccccc45)c3)cc2)c2ccc(-c3cccc4ccccc34)c3oc4ccccc4c23)cc1 ; c1cc(-c2ccc(N(c3cccc4ccccc34)c3ccc(-c4cccc5ccccc45)c4oc5ccccc5c34)cc2)cc(-c2cc3ccccc3c3ccccc23)c1 ; c1cc(-c2ccc(N(c3ccc4ccccc4c3)c3ccc(-c4cccc5ccccc45)c4oc5ccccc5c34)cc2)cc(-c2cc3ccccc3c3ccccc23)c1 ; c1cc(-c2ccc(N(c3cc4ccccc4c4ccccc34)c3ccc(-c4cccc5ccccc45)c4oc5ccccc5c34)cc2)cc(-c2cc3ccccc3c3ccccc23)c1]

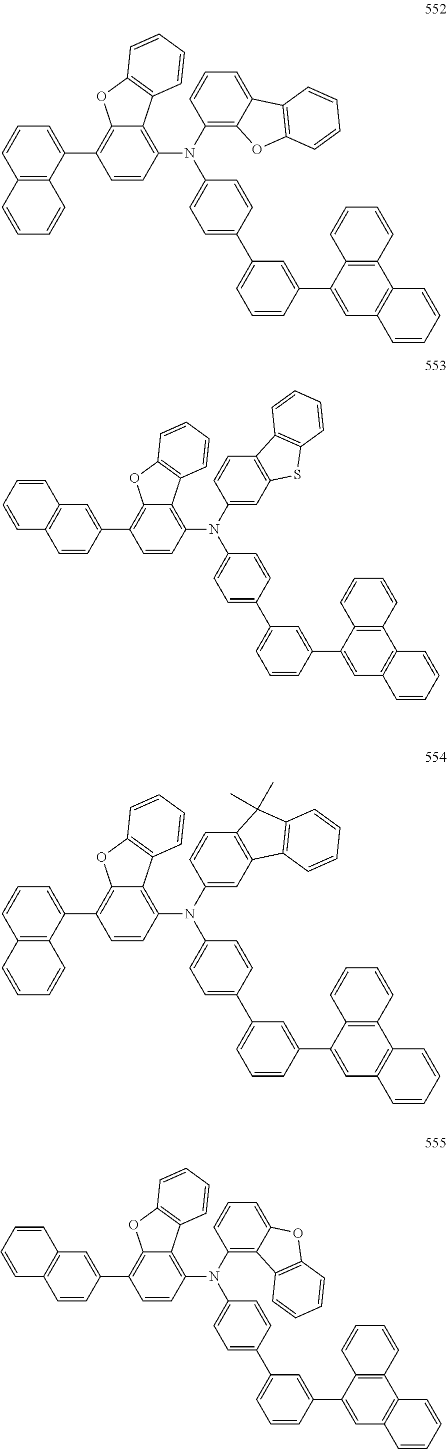 [SMILES: c1cc(-c2ccc(N(c3cccc4c3oc3ccccc34)c3ccc(-c4cccc5ccccc45)c4oc5ccccc5c34)cc2)cc(-c2cc3ccccc3c3ccccc23)c1 ; c1cc(-c2ccc(N(c3ccc4c(c3)sc3ccccc34)c3ccc(-c4ccc5ccccc5c4)c4oc5ccccc5c34)cc2)cc(-c2cc3ccccc3c3ccccc23)c1 ; CC1(C)c2ccccc2-c2cc(N(c3ccc(-c4cccc(-c5cc6ccccc6c6ccccc56)c4)cc3)c3ccc(-c4cccc5ccccc45)c4oc5ccccc5c34)ccc21 ; c1cc(-c2ccc(N(c3cccc4oc5ccccc5c34)c3ccc(-c4ccc5ccccc5c4)c4oc5ccccc5c34)cc2)cc(-c2cc3ccccc3c3ccccc23)c1]

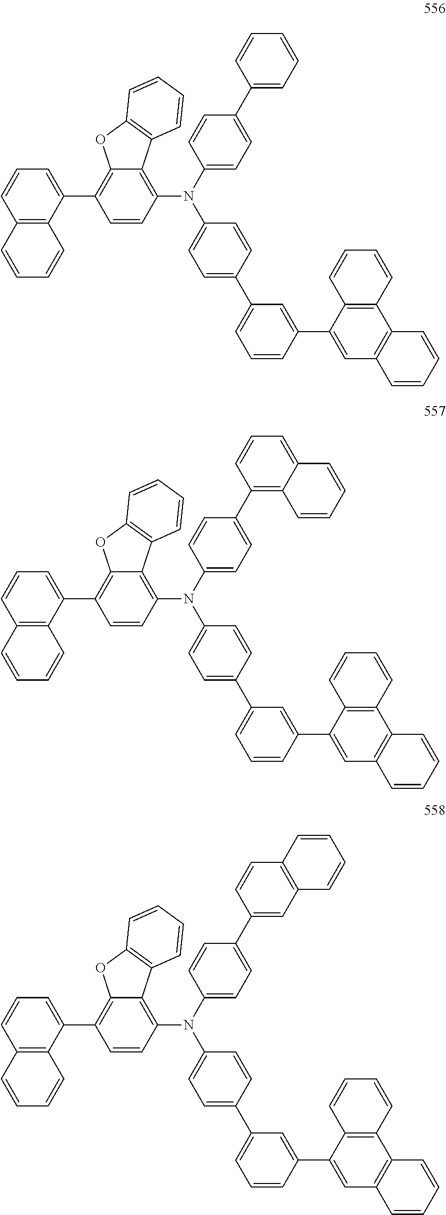 [SMILES: c1ccc(-c2ccc(N(c3ccc(-c4cccc(-c5cc6ccccc6c6ccccc56)c4)cc3)c3ccc(-c4cccc5ccccc45)c4oc5ccccc5c34)cc2)cc1 ; c1cc(-c2ccc(N(c3ccc(-c4cccc5ccccc45)cc3)c3ccc(-c4cccc5ccccc45)c4oc5ccccc5c34)cc2)cc(-c2cc3ccccc3c3ccccc23)c1 ; c1cc(-c2ccc(N(c3ccc(-c4ccc5ccccc5c4)cc3)c3ccc(-c4cccc5ccccc45)c4oc5ccccc5c34)cc2)cc(-c2cc3ccccc3c3ccccc23)c1]

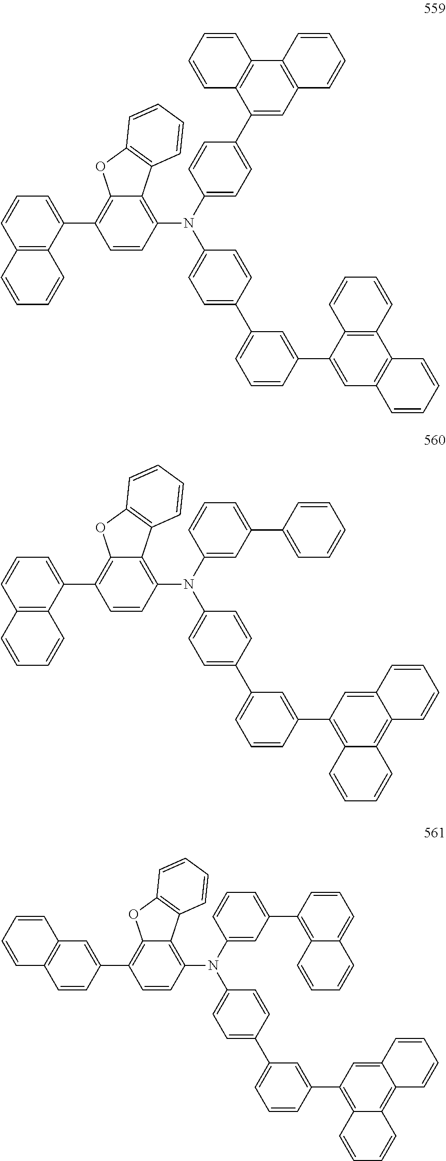 [SMILES: c1cc(-c2ccc(N(c3ccc(-c4cc5ccccc5c5ccccc45)cc3)c3ccc(-c4cccc5ccccc45)c4oc5ccccc5c34)cc2)cc(-c2cc3ccccc3c3ccccc23)c1 ; c1ccc(-c2cccc(N(c3ccc(-c4cccc(-c5cc6ccccc6c6ccccc56)c4)cc3)c3ccc(-c4cccc5ccccc45)c4oc5ccccc5c34)c2)cc1 ; c1cc(-c2ccc(N(c3cccc(-c4cccc5ccccc45)c3)c3ccc(-c4ccc5ccccc5c4)c4oc5ccccc5c34)cc2)cc(-c2cc3ccccc3c3ccccc23)c1]

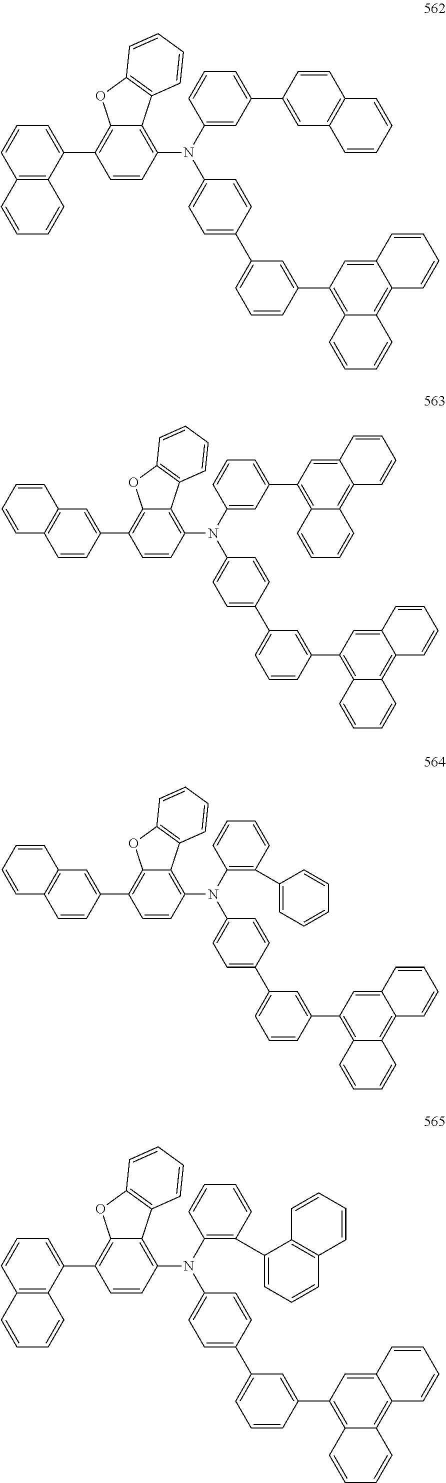 [SMILES: c1cc(-c2ccc(N(c3cccc(-c4ccc5ccccc5c4)c3)c3ccc(-c4cccc5ccccc45)c4oc5ccccc5c34)cc2)cc(-c2cc3ccccc3c3ccccc23)c1 ; c1cc(-c2ccc(N(c3cccc(-c4cc5ccccc5c5ccccc45)c3)c3ccc(-c4ccc5ccccc5c4)c4oc5ccccc5c34)cc2)cc(-c2cc3ccccc3c3ccccc23)c1 ; c1ccc(-c2ccccc2N(c2ccc(-c3cccc(-c4cc5ccccc5c5ccccc45)c3)cc2)c2ccc(-c3ccc4ccccc4c3)c3oc4ccccc4c23)cc1 ; c1cc(-c2ccc(N(c3ccccc3-c3cccc4ccccc34)c3ccc(-c4cccc5ccccc45)c4oc5ccccc5c34)cc2)cc(-c2cc3ccccc3c3ccccc23)c1]

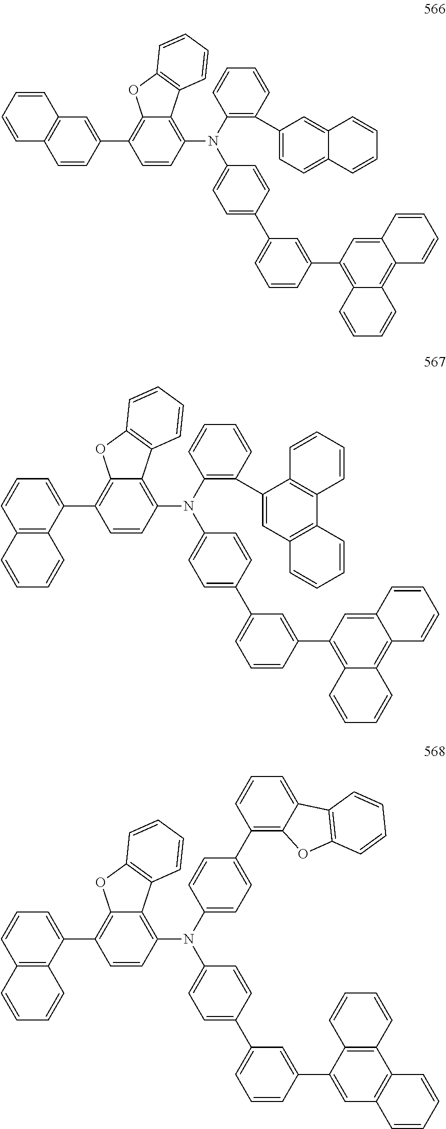 [SMILES: c1cc(-c2ccc(N(c3ccccc3-c3ccc4ccccc4c3)c3ccc(-c4ccc5ccccc5c4)c4oc5ccccc5c34)cc2)cc(-c2cc3ccccc3c3ccccc23)c1 ; c1cc(-c2ccc(N(c3ccccc3-c3cc4ccccc4c4ccccc34)c3ccc(-c4cccc5ccccc45)c4oc5ccccc5c34)cc2)cc(-c2cc3ccccc3c3ccccc23)c1 ; c1cc(-c2ccc(N(c3ccc(-c4cccc5c4oc4ccccc45)cc3)c3ccc(-c4cccc5ccccc45)c4oc5ccccc5c34)cc2)cc(-c2cc3ccccc3c3ccccc23)c1]

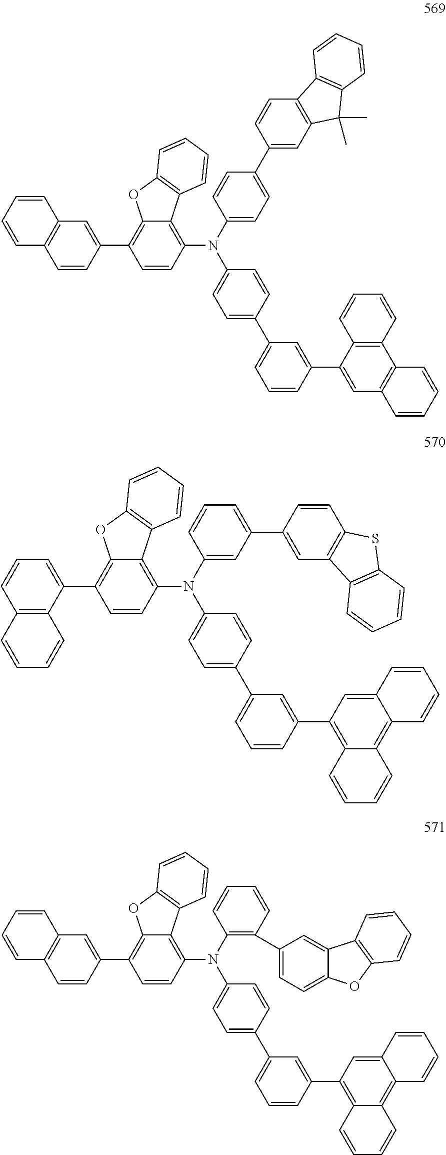 [SMILES: CC1(C)c2ccccc2-c2ccc(-c3ccc(N(c4ccc(-c5cccc(-c6cc7ccccc7c7ccccc67)c5)cc4)c4ccc(-c5ccc6ccccc6c5)c5oc6ccccc6c45)cc3)cc21 ; c1cc(-c2ccc(N(c3cccc(-c4ccc5sc6ccccc6c5c4)c3)c3ccc(-c4cccc5ccccc45)c4oc5ccccc5c34)cc2)cc(-c2cc3ccccc3c3ccccc23)c1 ; c1cc(-c2ccc(N(c3ccccc3-c3ccc4oc5ccccc5c4c3)c3ccc(-c4ccc5ccccc5c4)c4oc5ccccc5c34)cc2)cc(-c2cc3ccccc3c3ccccc23)c1]

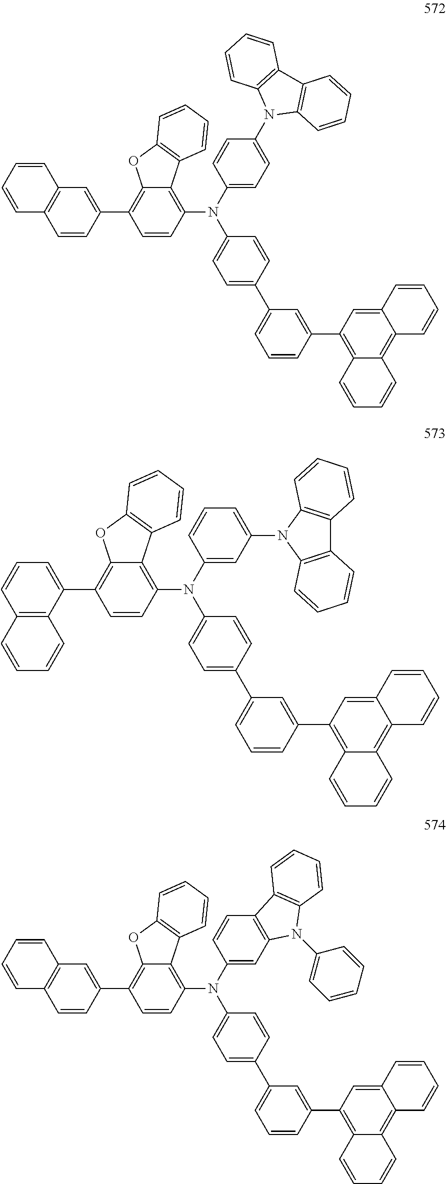 [SMILES: c1cc(-c2ccc(N(c3ccc(-n4c5ccccc5c5ccccc54)cc3)c3ccc(-c4ccc5ccccc5c4)c4oc5ccccc5c34)cc2)cc(-c2cc3ccccc3c3ccccc23)c1 ; c1cc(-c2ccc(N(c3cccc(-n4c5ccccc5c5ccccc54)c3)c3ccc(-c4cccc5ccccc45)c4oc5ccccc5c34)cc2)cc(-c2cc3ccccc3c3ccccc23)c1 ; c1ccc(-n2c3ccccc3c3ccc(N(c4ccc(-c5cccc(-c6cc7ccccc7c7ccccc67)c5)cc4)c4ccc(-c5ccc6ccccc6c5)c5oc6ccccc6c45)cc32)cc1]

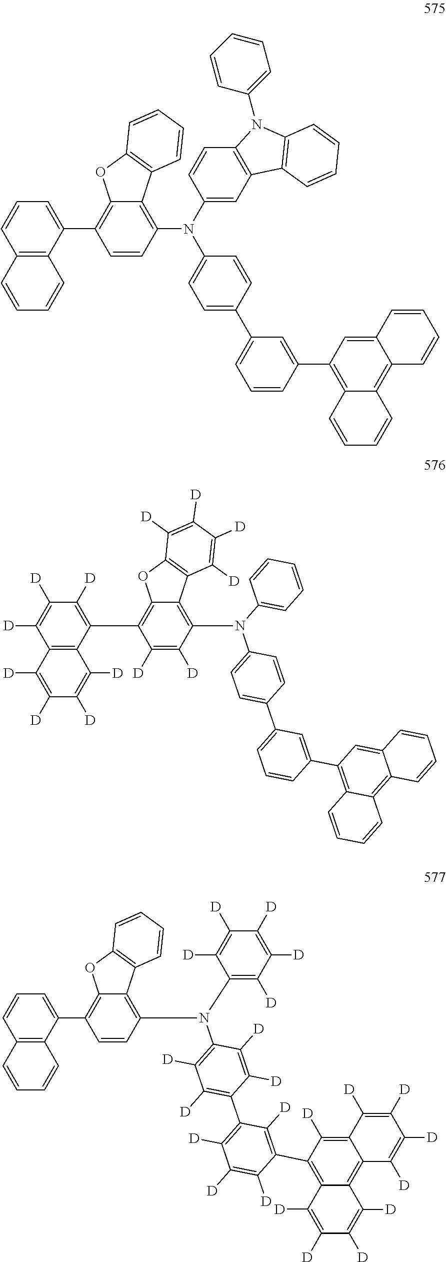

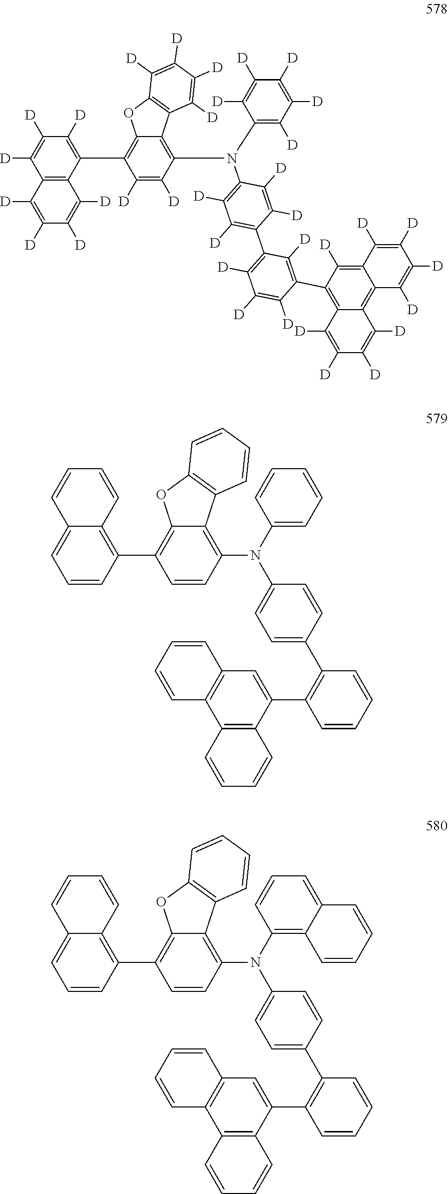 [SMILES: [2H]c1c([2H])c([2H])c(N(c2c([2H])c([2H])c(-c3c([2H])c([2H])c([2H])c(-c4c([2H])c5c([2H])c([2H])c([2H])c([2H])c5c5c([2H])c([2H])c([2H])c([2H])c45)c3[2H])c([2H])c2[2H])c2c([2H])c([2H])c(-c3c([2H])c([2H])c([2H])c4c([2H])c([2H])c([2H])c([2H])c34)c3oc4c([2H])c([2H])c([2H])c([2H])c4c23)c([2H])c1[2H] ; c1ccc(N(c2ccc(-c3ccccc3-c3cc4ccccc4c4ccccc34)cc2)c2ccc(-c3cccc4ccccc34)c3oc4ccccc4c23)cc1 ; c1ccc(-c2cc3ccccc3c3ccccc23)c(-c2ccc(N(c3cccc4ccccc34)c3ccc(-c4cccc5ccccc45)c4oc5ccccc5c34)cc2)c1]

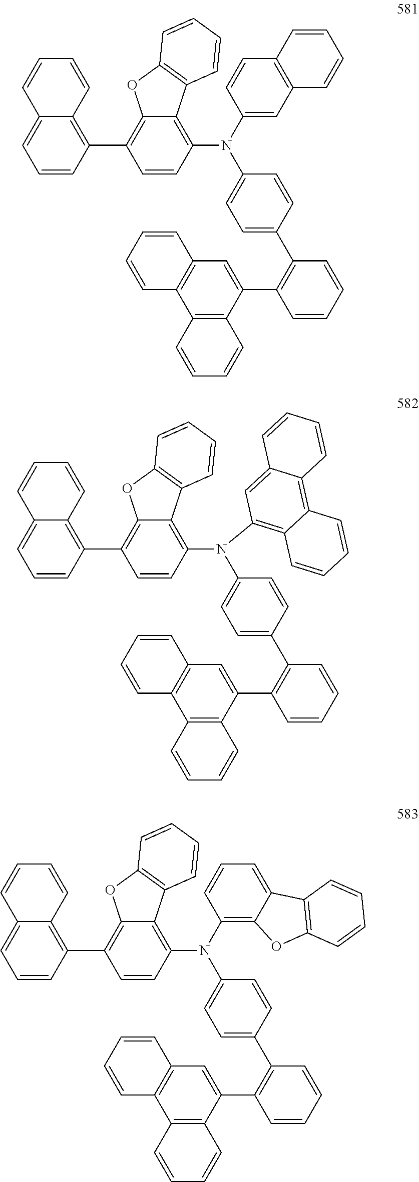

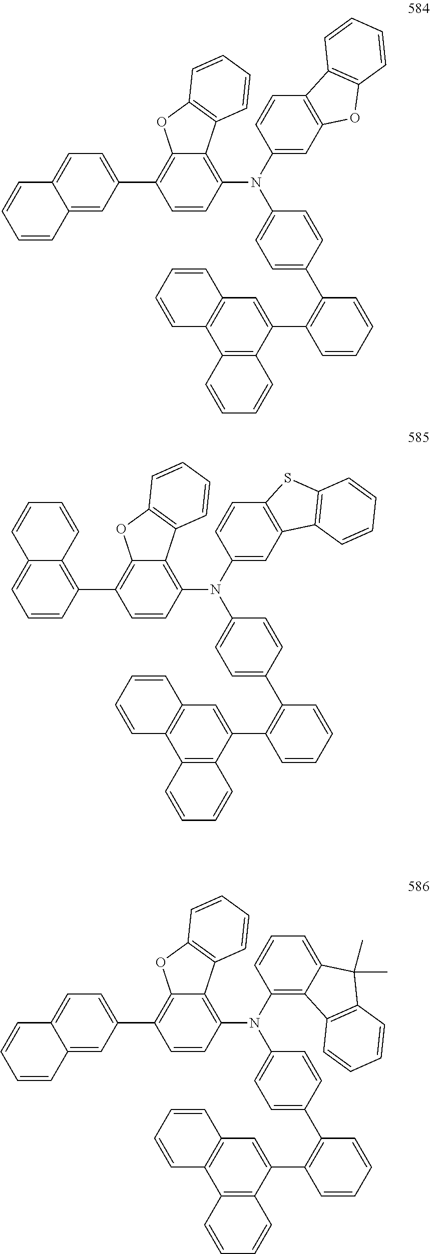 [SMILES: c1ccc(-c2cc3ccccc3c3ccccc23)c(-c2ccc(N(c3ccc4c(c3)oc3ccccc34)c3ccc(-c4ccc5ccccc5c4)c4oc5ccccc5c34)cc2)c1 ; c1ccc(-c2cc3ccccc3c3ccccc23)c(-c2ccc(N(c3ccc4sc5ccccc5c4c3)c3ccc(-c4cccc5ccccc45)c4oc5ccccc5c34)cc2)c1 ; CC1(C)c2ccccc2-c2c(N(c3ccc(-c4ccccc4-c4cc5ccccc5c5ccccc45)cc3)c3ccc(-c4ccc5ccccc5c4)c4oc5ccccc5c34)cccc21]

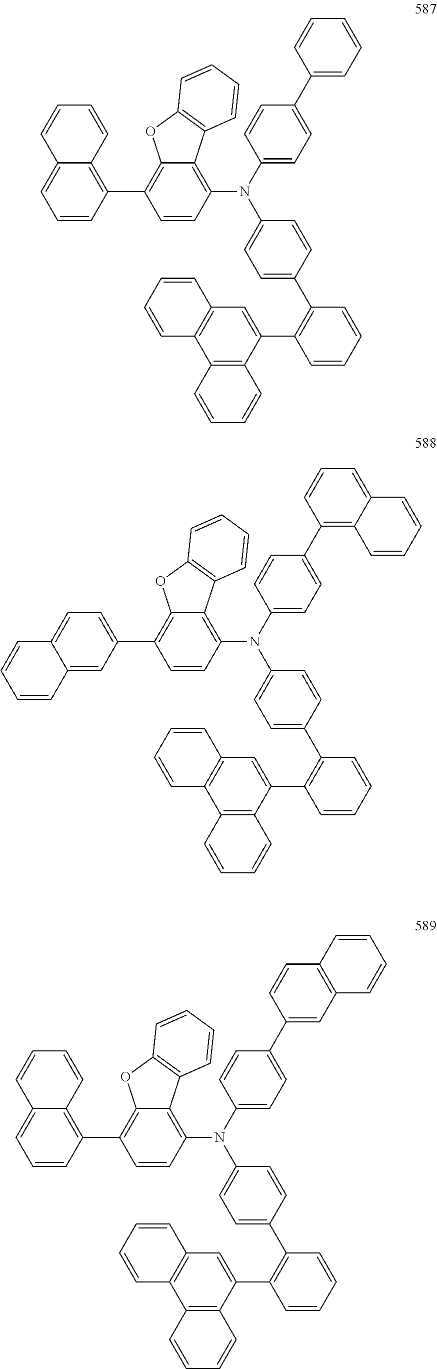 [SMILES: c1ccc(-c2ccc(N(c3ccc(-c4ccccc4-c4cc5ccccc5c5ccccc45)cc3)c3ccc(-c4cccc5ccccc45)c4oc5ccccc5c34)cc2)cc1 ; c1ccc(-c2cc3ccccc3c3ccccc23)c(-c2ccc(N(c3ccc(-c4cccc5ccccc45)cc3)c3ccc(-c4ccc5ccccc5c4)c4oc5ccccc5c34)cc2)c1 ; c1ccc(-c2cc3ccccc3c3ccccc23)c(-c2ccc(N(c3ccc(-c4ccc5ccccc5c4)cc3)c3ccc(-c4cccc5ccccc45)c4oc5ccccc5c34)cc2)c1]

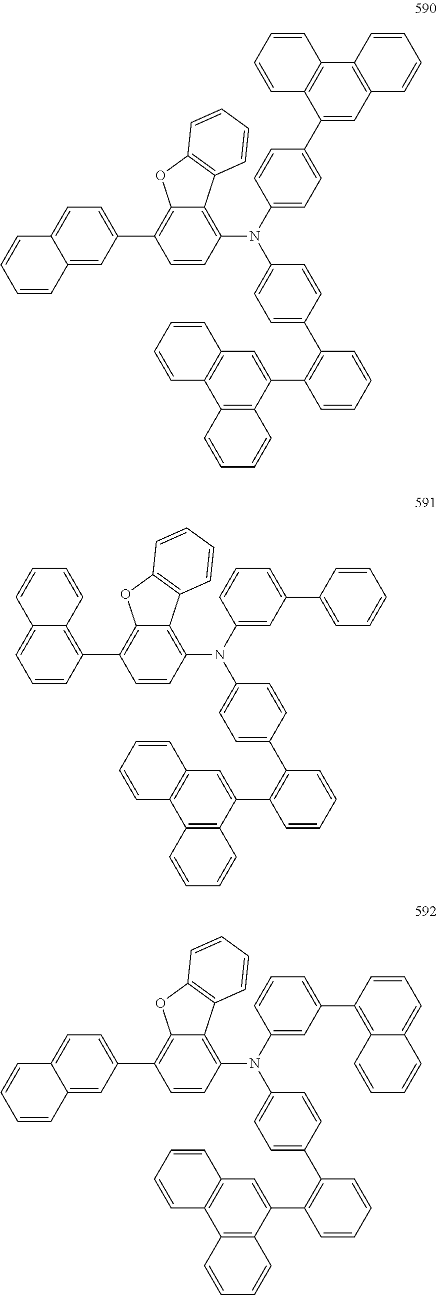 [SMILES: c1ccc(-c2cc3ccccc3c3ccccc23)c(-c2ccc(N(c3ccc(-c4cc5ccccc5c5ccccc45)cc3)c3ccc(-c4ccc5ccccc5c4)c4oc5ccccc5c34)cc2)c1 ; c1ccc(-c2cccc(N(c3ccc(-c4ccccc4-c4cc5ccccc5c5ccccc45)cc3)c3ccc(-c4cccc5ccccc45)c4oc5ccccc5c34)c2)cc1 ; c1cc(-c2cccc3ccccc23)cc(N(c2ccc(-c3ccccc3-c3cc4ccccc4c4ccccc34)cc2)c2ccc(-c3ccc4ccccc4c3)c3oc4ccccc4c23)c1]

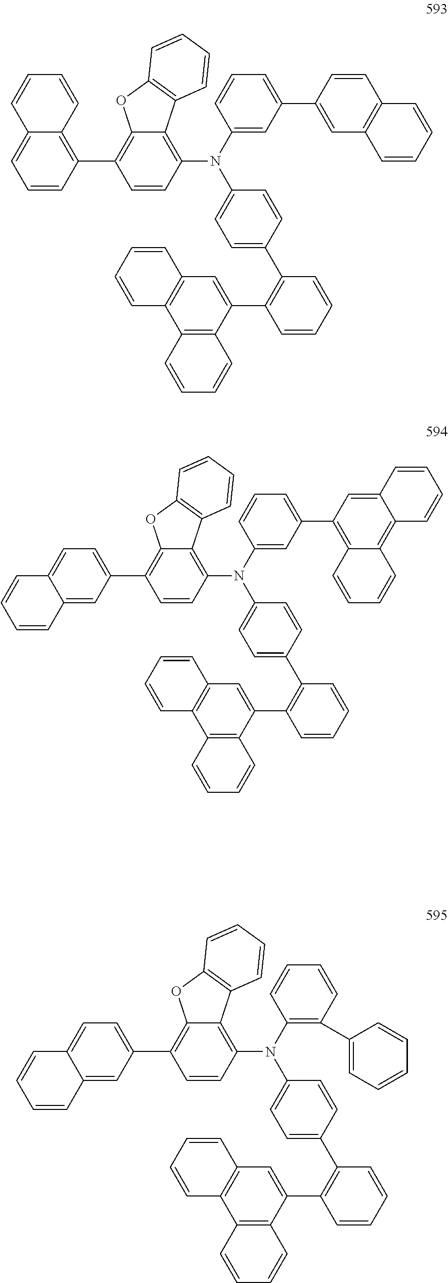 [SMILES: c1cc(-c2ccc3ccccc3c2)cc(N(c2ccc(-c3ccccc3-c3cc4ccccc4c4ccccc34)cc2)c2ccc(-c3cccc4ccccc34)c3oc4ccccc4c23)c1 ; c1cc(-c2cc3ccccc3c3ccccc23)cc(N(c2ccc(-c3ccccc3-c3cc4ccccc4c4ccccc34)cc2)c2ccc(-c3ccc4ccccc4c3)c3oc4ccccc4c23)c1 ; c1ccc(-c2ccccc2N(c2ccc(-c3ccccc3-c3cc4ccccc4c4ccccc34)cc2)c2ccc(-c3ccc4ccccc4c3)c3oc4ccccc4c23)cc1]

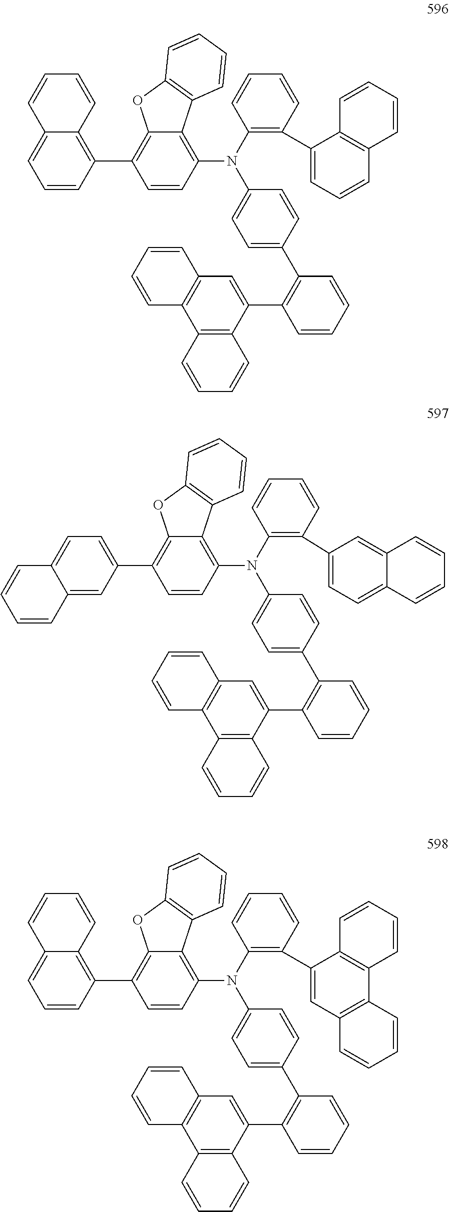 [SMILES: c1ccc(-c2cc3ccccc3c3ccccc23)c(-c2ccc(N(c3ccccc3-c3cccc4ccccc34)c3ccc(-c4cccc5ccccc45)c4oc5ccccc5c34)cc2)c1 ; c1ccc(-c2cc3ccccc3c3ccccc23)c(-c2ccc(N(c3ccccc3-c3ccc4ccccc4c3)c3ccc(-c4ccc5ccccc5c4)c4oc5ccccc5c34)cc2)c1 ; c1ccc(-c2cc3ccccc3c3ccccc23)c(-c2ccc(N(c3ccccc3-c3cc4ccccc4c4ccccc34)c3ccc(-c4cccc5ccccc45)c4oc5ccccc5c34)cc2)c1]

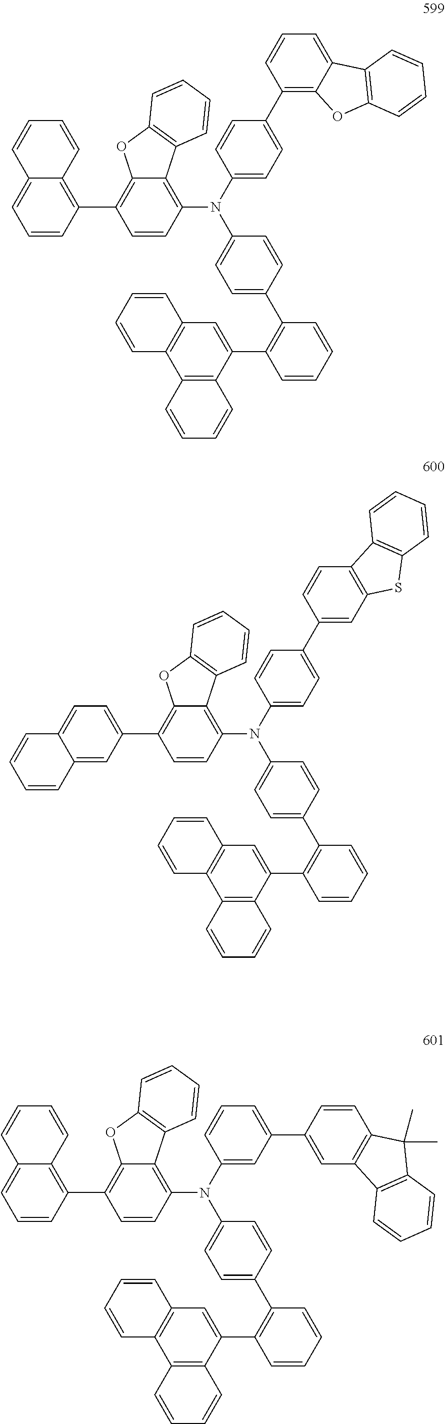 [SMILES: c1ccc(-c2cc3ccccc3c3ccccc23)c(-c2ccc(N(c3ccc(-c4cccc5c4oc4ccccc45)cc3)c3ccc(-c4cccc5ccccc45)c4oc5ccccc5c34)cc2)c1 ; c1ccc(-c2cc3ccccc3c3ccccc23)c(-c2ccc(N(c3ccc(-c4ccc5c(c4)sc4ccccc45)cc3)c3ccc(-c4ccc5ccccc5c4)c4oc5ccccc5c34)cc2)c1 ; CC1(C)c2ccccc2-c2cc(-c3cccc(N(c4ccc(-c5ccccc5-c5cc6ccccc6c6ccccc56)cc4)c4ccc(-c5cccc6ccccc56)c5oc6ccccc6c45)c3)ccc21]

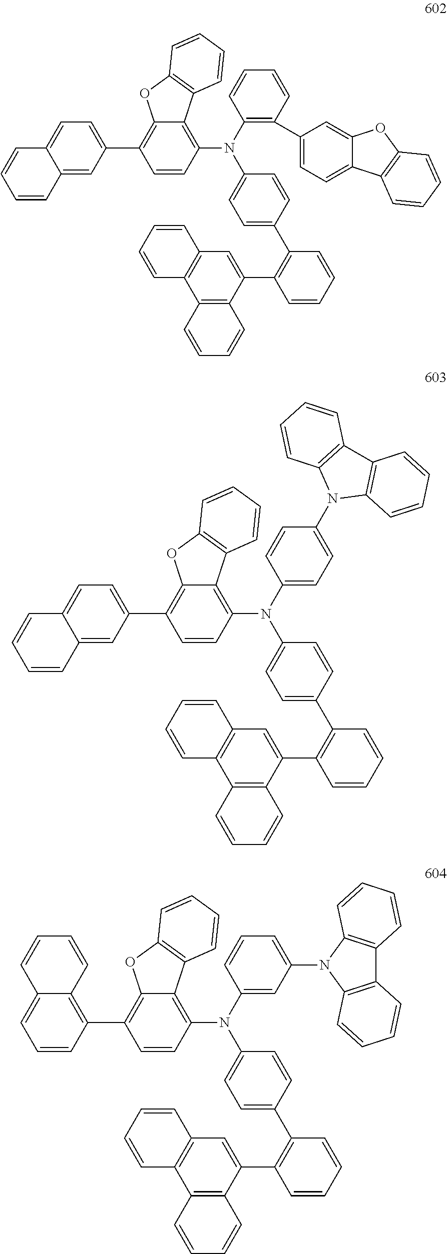 [SMILES: c1ccc(-c2cc3ccccc3c3ccccc23)c(-c2ccc(N(c3ccccc3-c3ccc4c(c3)oc3ccccc34)c3ccc(-c4ccc5ccccc5c4)c4oc5ccccc5c34)cc2)c1 ; c1ccc(-c2cc3ccccc3c3ccccc23)c(-c2ccc(N(c3ccc(-n4c5ccccc5c5ccccc54)cc3)c3ccc(-c4ccc5ccccc5c4)c4oc5ccccc5c34)cc2)c1 ; c1cc(N(c2ccc(-c3ccccc3-c3cc4ccccc4c4ccccc34)cc2)c2ccc(-c3cccc4ccccc34)c3oc4ccccc4c23)cc(-n2c3ccccc3c3ccccc32)c1]

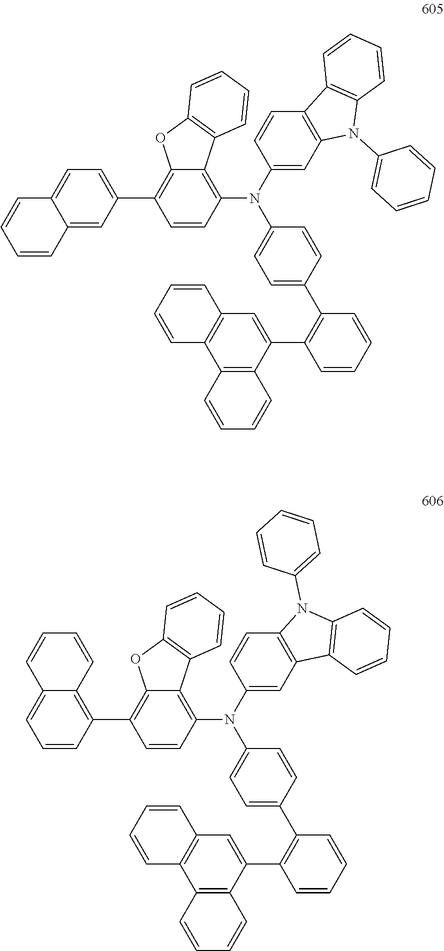 [SMILES: c1ccc(-n2c3ccccc3c3ccc(N(c4ccc(-c5ccccc5-c5cc6ccccc6c6ccccc56)cc4)c4ccc(-c5ccc6ccccc6c5)c5oc6ccccc6c45)cc32)cc1 ; c1ccc(-n2c3ccccc3c3cc(N(c4ccc(-c5ccccc5-c5cc6ccccc6c6ccccc56)cc4)c4ccc(-c5cccc6ccccc56)c5oc6ccccc6c45)ccc32)cc1]

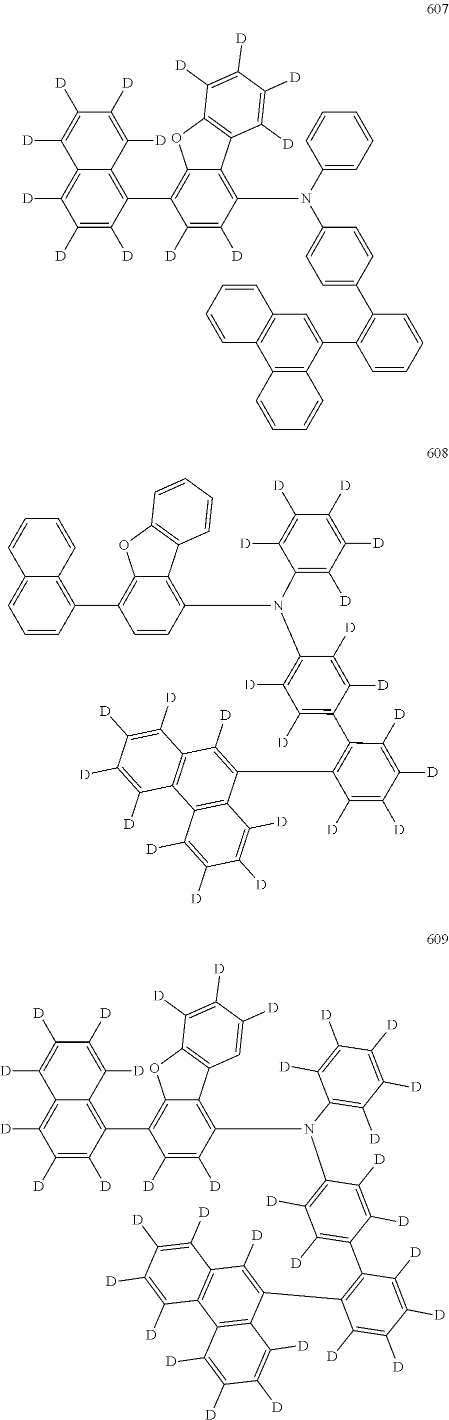

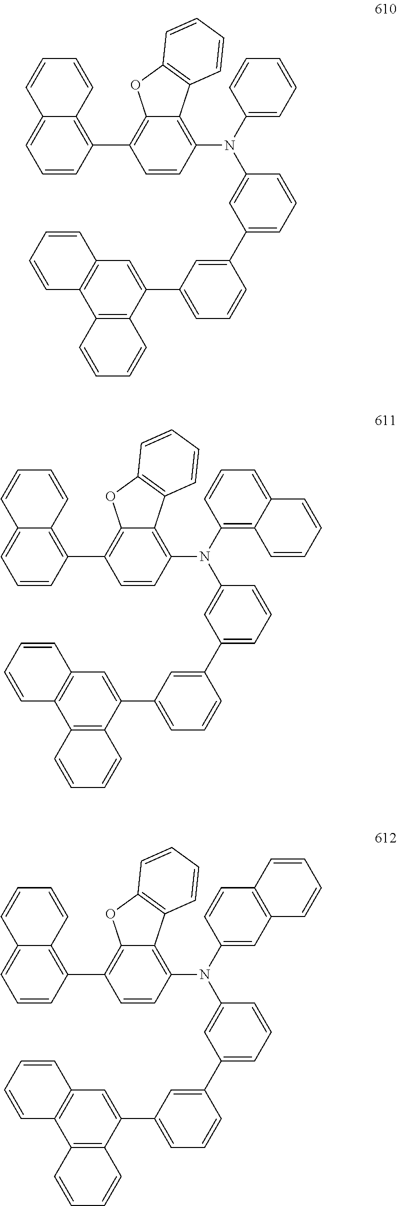 [SMILES: c1ccc(N(c2cccc(-c3cccc(-c4cc5ccccc5c5ccccc45)c3)c2)c2ccc(-c3cccc4ccccc34)c3oc4ccccc4c23)cc1 ; c1cc(-c2cccc(N(c3cccc4ccccc34)c3ccc(-c4cccc5ccccc45)c4oc5ccccc5c34)c2)cc(-c2cc3ccccc3c3ccccc23)c1 ; c1cc(-c2cccc(N(c3ccc4ccccc4c3)c3ccc(-c4cccc5ccccc45)c4oc5ccccc5c34)c2)cc(-c2cc3ccccc3c3ccccc23)c1]

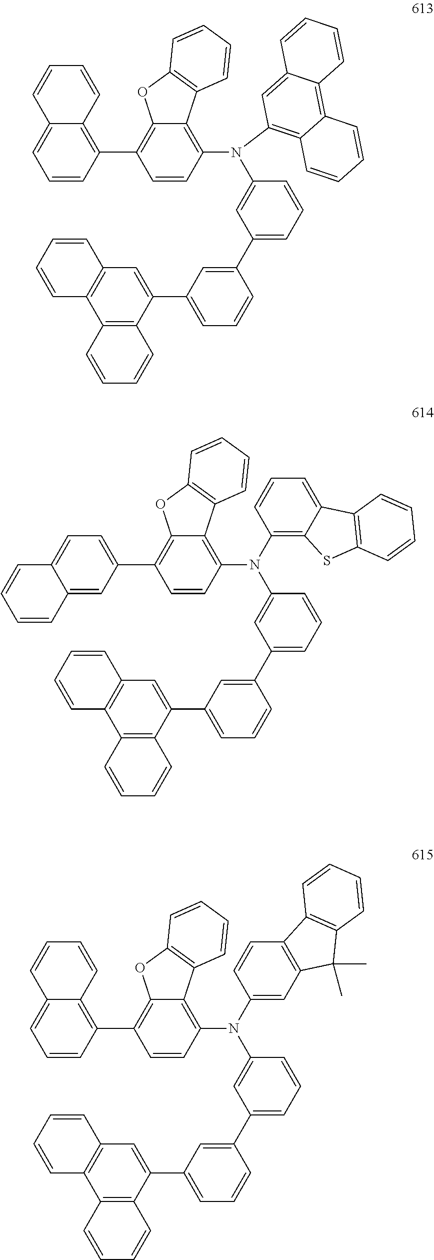 [SMILES: c1cc(-c2cccc(N(c3cc4ccccc4c4ccccc34)c3ccc(-c4cccc5ccccc45)c4oc5ccccc5c34)c2)cc(-c2cc3ccccc3c3ccccc23)c1 ; c1cc(-c2cccc(N(c3cccc4c3sc3ccccc34)c3ccc(-c4ccc5ccccc5c4)c4oc5ccccc5c34)c2)cc(-c2cc3ccccc3c3ccccc23)c1 ; CC1(C)c2ccccc2-c2ccc(N(c3cccc(-c4cccc(-c5cc6ccccc6c6ccccc56)c4)c3)c3ccc(-c4cccc5ccccc45)c4oc5ccccc5c34)cc21]

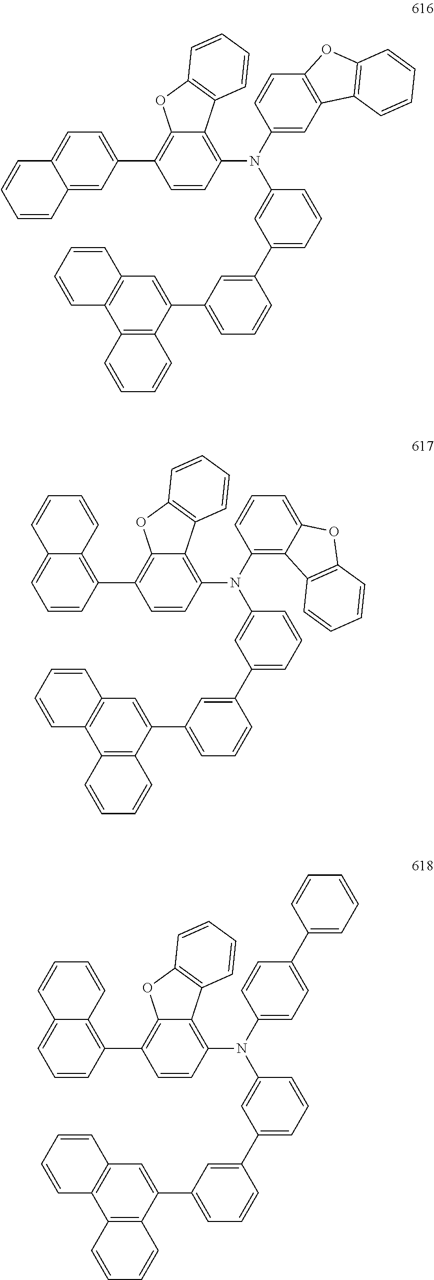 [SMILES: c1cc(-c2cccc(N(c3ccc4oc5ccccc5c4c3)c3ccc(-c4ccc5ccccc5c4)c4oc5ccccc5c34)c2)cc(-c2cc3ccccc3c3ccccc23)c1 ; c1cc(-c2cccc(N(c3cccc4oc5ccccc5c34)c3ccc(-c4cccc5ccccc45)c4oc5ccccc5c34)c2)cc(-c2cc3ccccc3c3ccccc23)c1 ; c1ccc(-c2ccc(N(c3cccc(-c4cccc(-c5cc6ccccc6c6ccccc56)c4)c3)c3ccc(-c4cccc5ccccc45)c4oc5ccccc5c34)cc2)cc1]

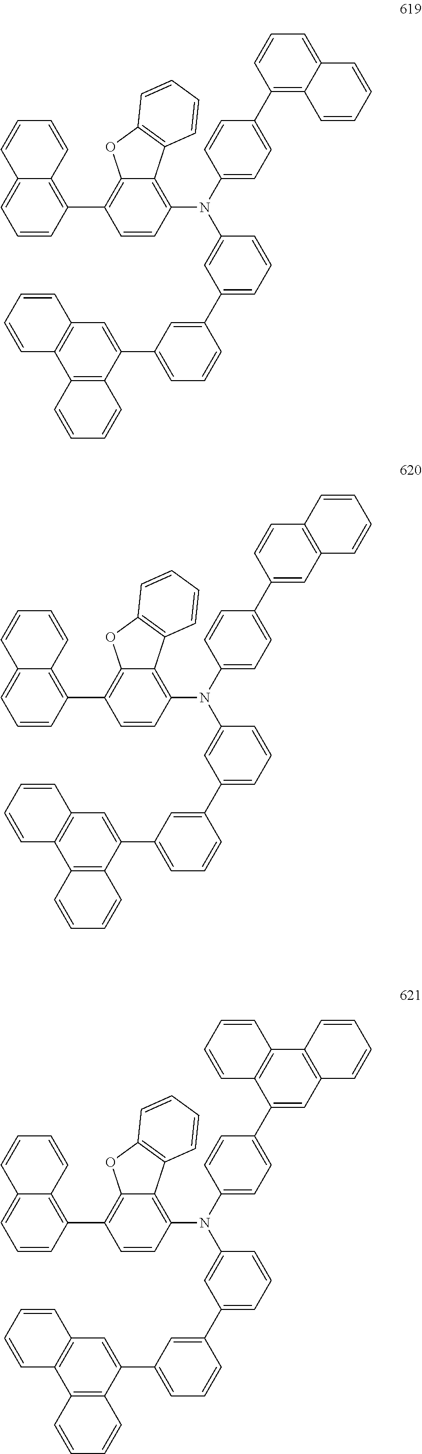 [SMILES: c1cc(-c2cccc(N(c3ccc(-c4cccc5ccccc45)cc3)c3ccc(-c4cccc5ccccc45)c4oc5ccccc5c34)c2)cc(-c2cc3ccccc3c3ccccc23)c1 ; c1cc(-c2cccc(N(c3ccc(-c4ccc5ccccc5c4)cc3)c3ccc(-c4cccc5ccccc45)c4oc5ccccc5c34)c2)cc(-c2cc3ccccc3c3ccccc23)c1 ; c1cc(-c2cccc(N(c3ccc(-c4cc5ccccc5c5ccccc45)cc3)c3ccc(-c4cccc5ccccc45)c4oc5ccccc5c34)c2)cc(-c2cc3ccccc3c3ccccc23)c1]

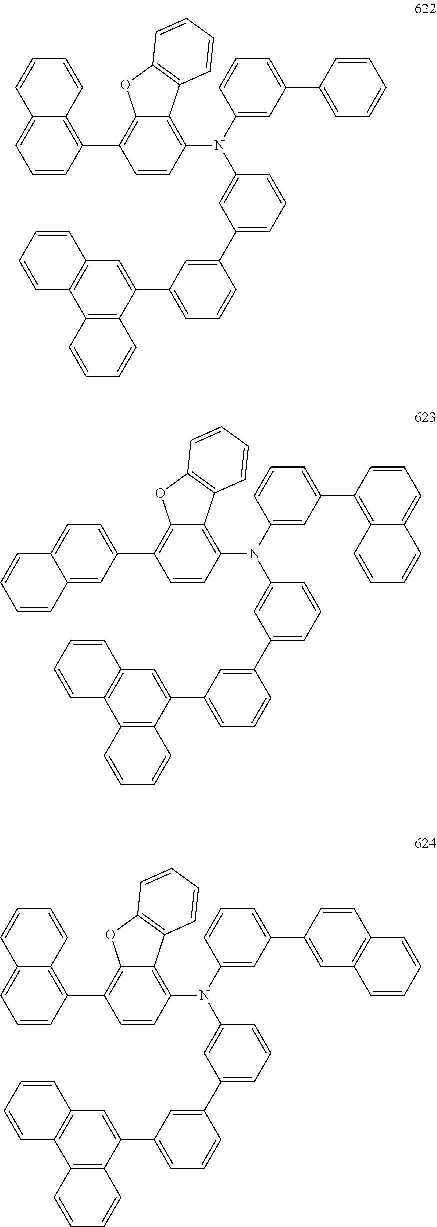 [SMILES: c1ccc(-c2cccc(N(c3cccc(-c4cccc(-c5cc6ccccc6c6ccccc56)c4)c3)c3ccc(-c4cccc5ccccc45)c4oc5ccccc5c34)c2)cc1 ; c1cc(-c2cccc(N(c3cccc(-c4cccc5ccccc45)c3)c3ccc(-c4ccc5ccccc5c4)c4oc5ccccc5c34)c2)cc(-c2cc3ccccc3c3ccccc23)c1 ; c1cc(-c2cccc(N(c3cccc(-c4ccc5ccccc5c4)c3)c3ccc(-c4cccc5ccccc45)c4oc5ccccc5c34)c2)cc(-c2cc3ccccc3c3ccccc23)c1]

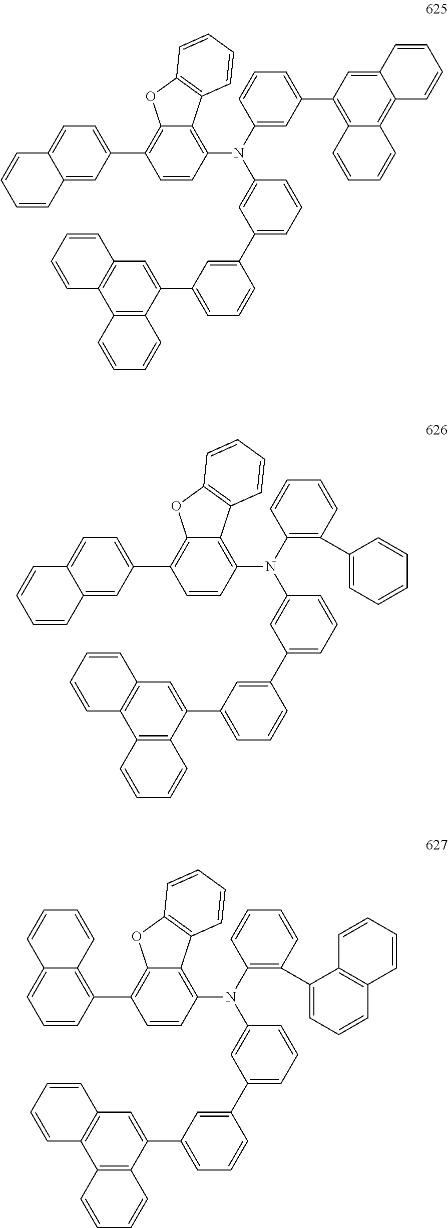 [SMILES: c1cc(-c2cccc(N(c3cccc(-c4cc5ccccc5c5ccccc45)c3)c3ccc(-c4ccc5ccccc5c4)c4oc5ccccc5c34)c2)cc(-c2cc3ccccc3c3ccccc23)c1 ; c1ccc(-c2ccccc2N(c2cccc(-c3cccc(-c4cc5ccccc5c5ccccc45)c3)c2)c2ccc(-c3ccc4ccccc4c3)c3oc4ccccc4c23)cc1 ; c1cc(-c2cccc(N(c3ccccc3-c3cccc4ccccc34)c3ccc(-c4cccc5ccccc45)c4oc5ccccc5c34)c2)cc(-c2cc3ccccc3c3ccccc23)c1]

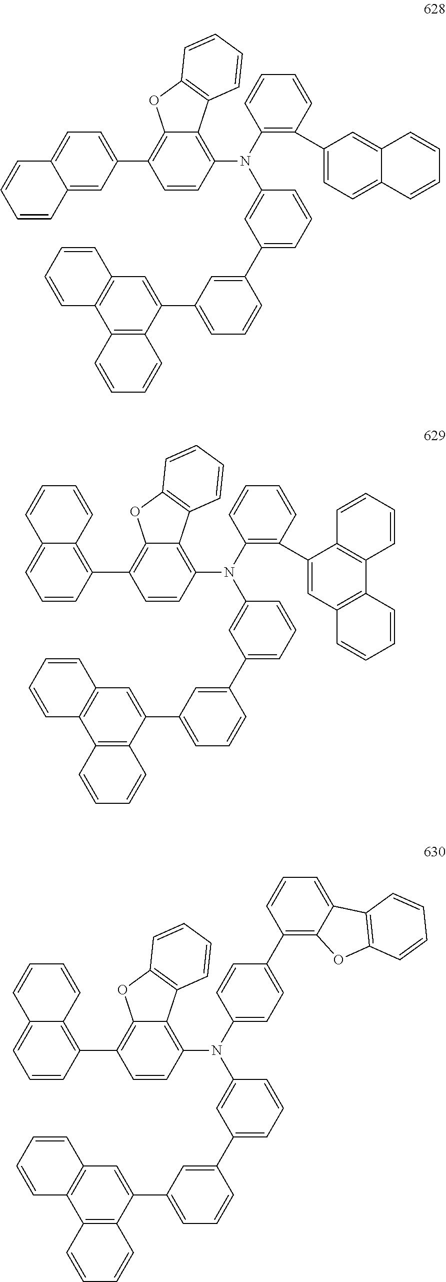 [SMILES: c1cc(-c2cccc(N(c3ccccc3-c3ccc4ccccc4c3)c3ccc(-c4ccc5ccccc5c4)c4oc5ccccc5c34)c2)cc(-c2cc3ccccc3c3ccccc23)c1 ; c1cc(-c2cccc(N(c3ccccc3-c3cc4ccccc4c4ccccc34)c3ccc(-c4cccc5ccccc45)c4oc5ccccc5c34)c2)cc(-c2cc3ccccc3c3ccccc23)c1 ; c1cc(-c2cccc(N(c3ccc(-c4cccc5c4oc4ccccc45)cc3)c3ccc(-c4cccc5ccccc45)c4oc5ccccc5c34)c2)cc(-c2cc3ccccc3c3ccccc23)c1]

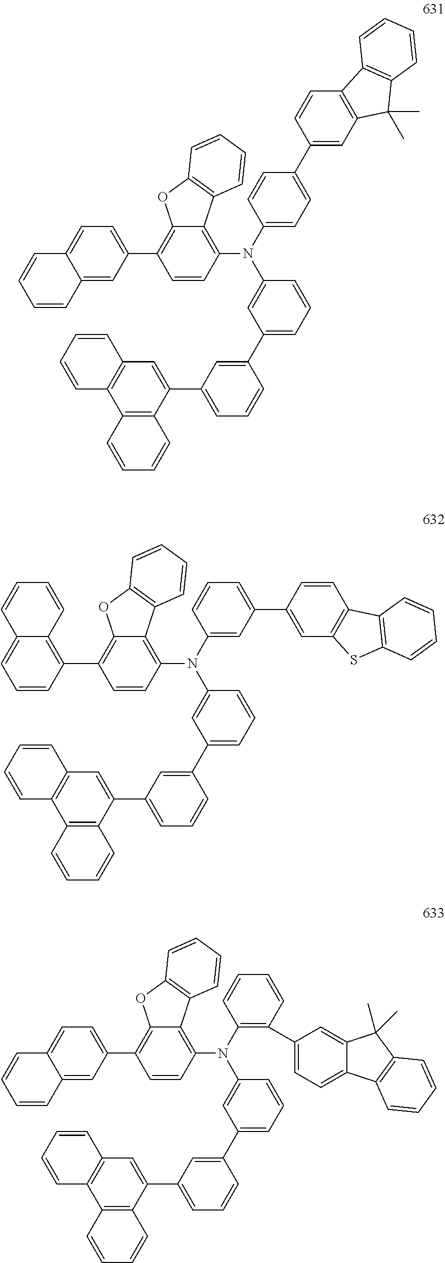 [SMILES: CC1(C)c2ccccc2-c2ccc(-c3ccc(N(c4cccc(-c5cccc(-c6cc7ccccc7c7ccccc67)c5)c4)c4ccc(-c5ccc6ccccc6c5)c5oc6ccccc6c45)cc3)cc21 ; c1cc(-c2cccc(N(c3cccc(-c4ccc5c(c4)sc4ccccc45)c3)c3ccc(-c4cccc5ccccc45)c4oc5ccccc5c34)c2)cc(-c2cc3ccccc3c3ccccc23)c1 ; CC1(C)c2ccccc2-c2ccc(-c3ccccc3N(c3cccc(-c4cccc(-c5cc6ccccc6c6ccccc56)c4)c3)c3ccc(-c4ccc5ccccc5c4)c4oc5ccccc5c34)cc21]

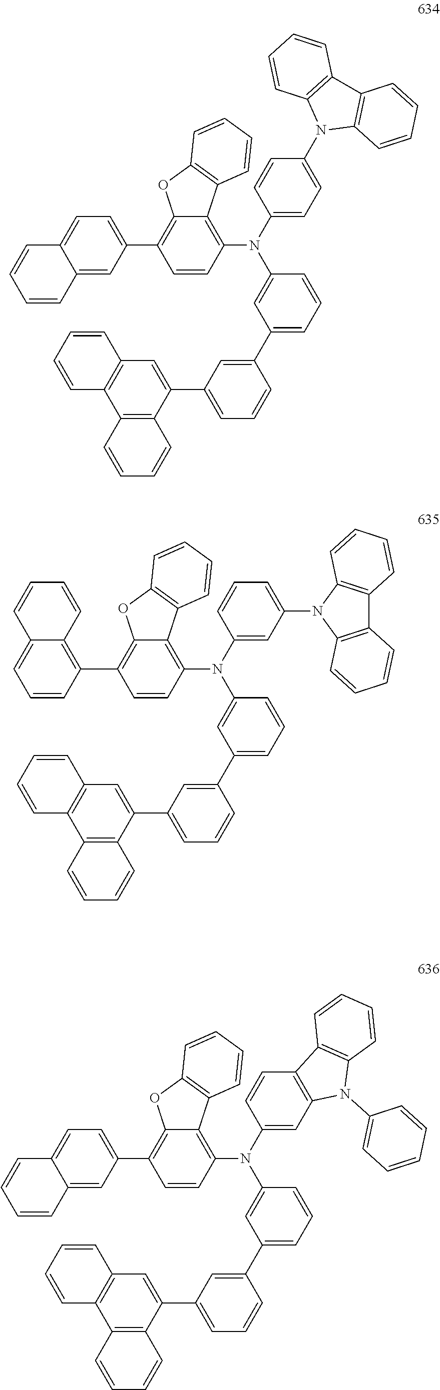 [SMILES: c1cc(-c2cccc(N(c3ccc(-n4c5ccccc5c5ccccc54)cc3)c3ccc(-c4ccc5ccccc5c4)c4oc5ccccc5c34)c2)cc(-c2cc3ccccc3c3ccccc23)c1 ; c1cc(-c2cccc(N(c3cccc(-n4c5ccccc5c5ccccc54)c3)c3ccc(-c4cccc5ccccc45)c4oc5ccccc5c34)c2)cc(-c2cc3ccccc3c3ccccc23)c1 ; c1ccc(-n2c3ccccc3c3ccc(N(c4cccc(-c5cccc(-c6cc7ccccc7c7ccccc67)c5)c4)c4ccc(-c5ccc6ccccc6c5)c5oc6ccccc6c45)cc32)cc1]

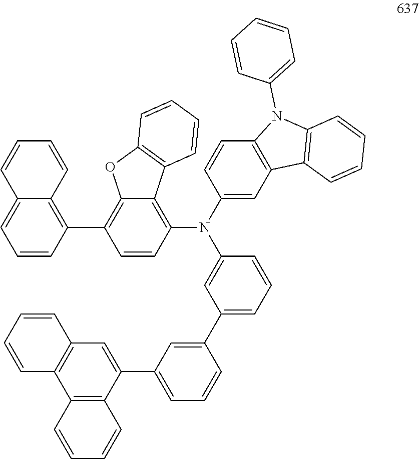 [SMILES: c1ccc(-n2c3ccccc3c3cc(N(c4cccc(-c5cccc(-c6cc7ccccc7c7ccccc67)c5)c4)c4ccc(-c5cccc6ccccc56)c5oc6ccccc6c45)ccc32)cc1]

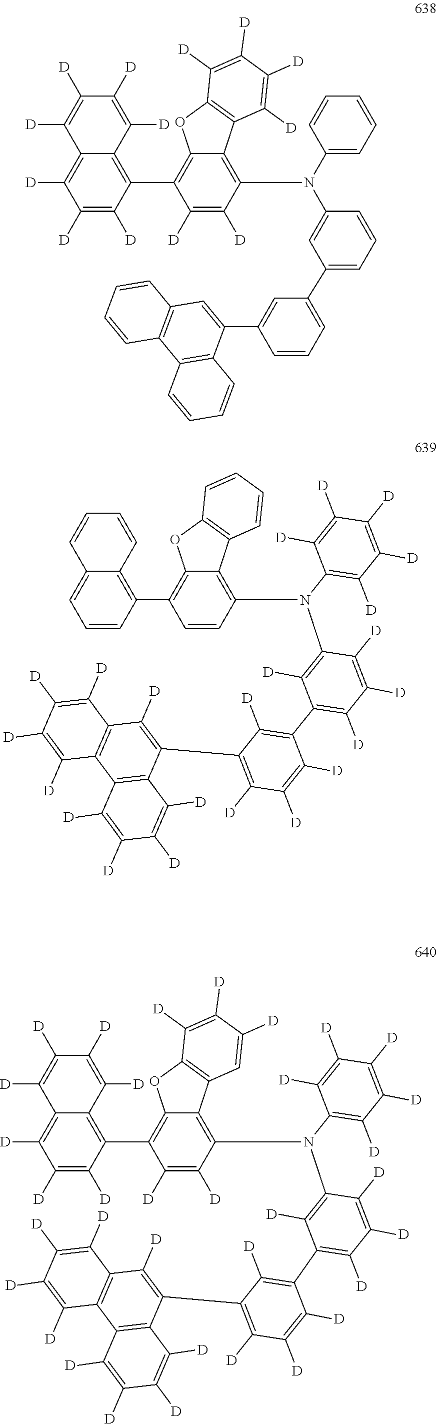

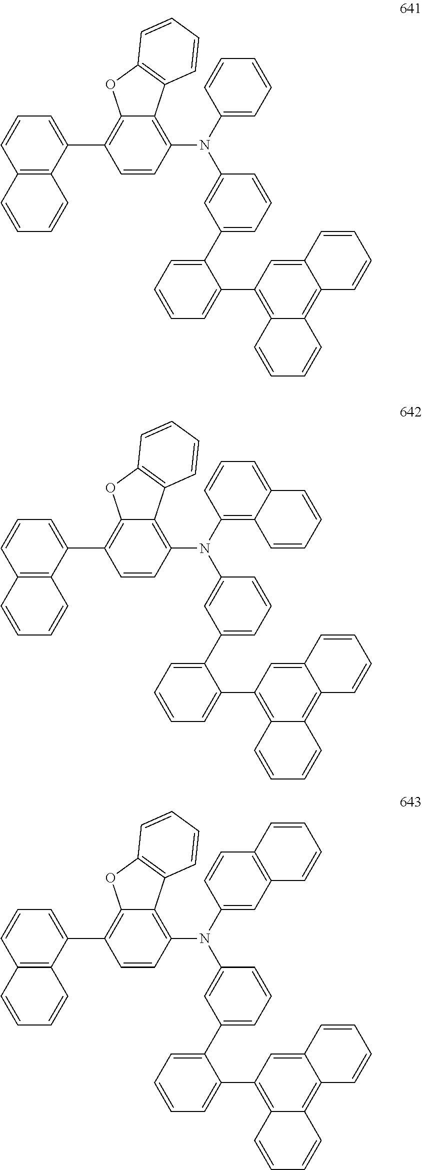

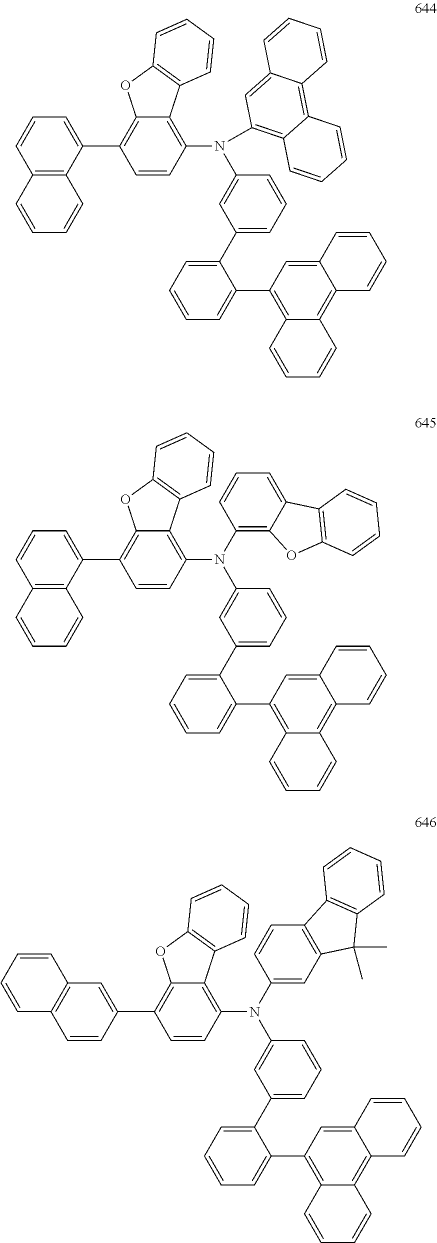 [SMILES: c1cc(-c2ccccc2-c2cc3ccccc3c3ccccc23)cc(N(c2cc3ccccc3c3ccccc23)c2ccc(-c3cccc4ccccc34)c3oc4ccccc4c23)c1 ; c1cc(-c2ccccc2-c2cc3ccccc3c3ccccc23)cc(N(c2cccc3c2oc2ccccc23)c2ccc(-c3cccc4ccccc34)c3oc4ccccc4c23)c1 ; CC1(C)c2ccccc2-c2ccc(N(c3cccc(-c4ccccc4-c4cc5ccccc5c5ccccc45)c3)c3ccc(-c4ccc5ccccc5c4)c4oc5ccccc5c34)cc21]

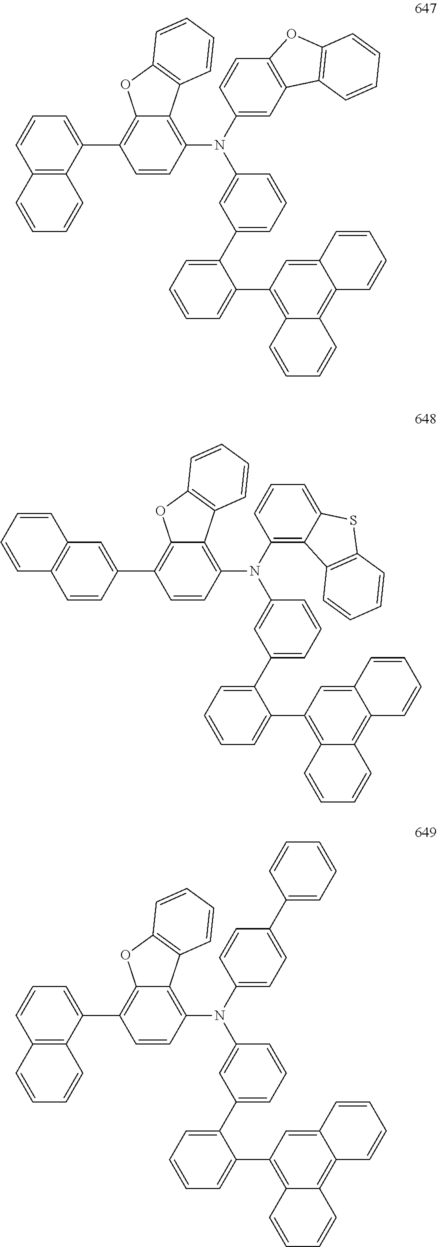

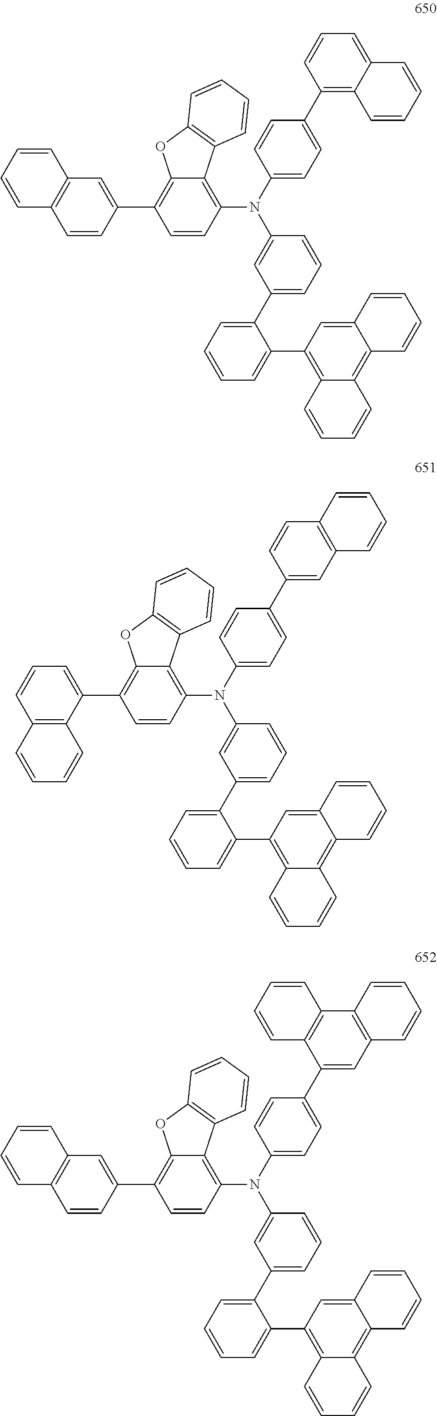 [SMILES: c1cc(-c2ccccc2-c2cc3ccccc3c3ccccc23)cc(N(c2ccc(-c3cccc4ccccc34)cc2)c2ccc(-c3ccc4ccccc4c3)c3oc4ccccc4c23)c1 ; c1cc(-c2ccccc2-c2cc3ccccc3c3ccccc23)cc(N(c2ccc(-c3ccc4ccccc4c3)cc2)c2ccc(-c3cccc4ccccc34)c3oc4ccccc4c23)c1 ; c1cc(-c2ccccc2-c2cc3ccccc3c3ccccc23)cc(N(c2ccc(-c3cc4ccccc4c4ccccc34)cc2)c2ccc(-c3ccc4ccccc4c3)c3oc4ccccc4c23)c1]

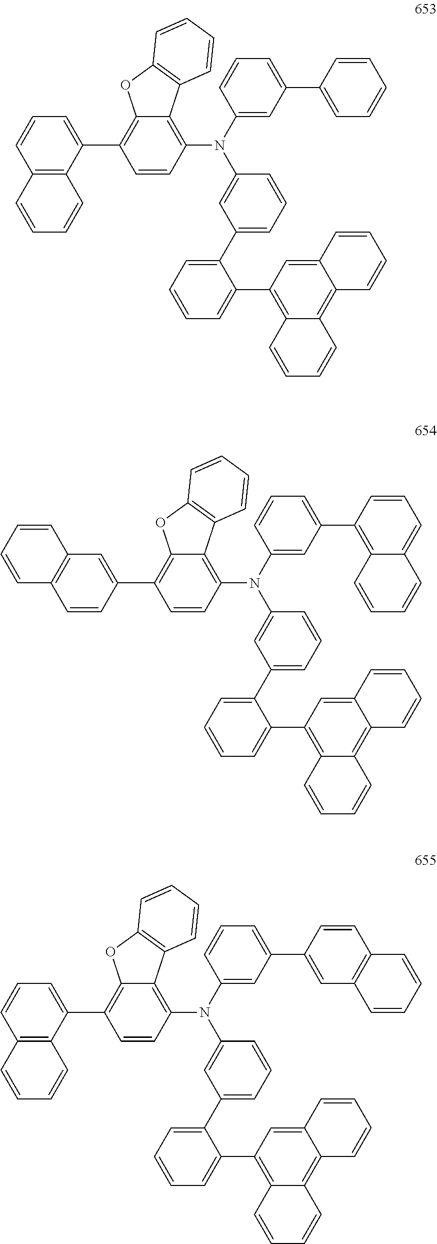 [SMILES: c1ccc(-c2cccc(N(c3cccc(-c4ccccc4-c4cc5ccccc5c5ccccc45)c3)c3ccc(-c4cccc5ccccc45)c4oc5ccccc5c34)c2)cc1 ; c1cc(-c2ccccc2-c2cc3ccccc3c3ccccc23)cc(N(c2cccc(-c3cccc4ccccc34)c2)c2ccc(-c3ccc4ccccc4c3)c3oc4ccccc4c23)c1 ; c1cc(-c2ccc3ccccc3c2)cc(N(c2cccc(-c3ccccc3-c3cc4ccccc4c4ccccc34)c2)c2ccc(-c3cccc4ccccc34)c3oc4ccccc4c23)c1]

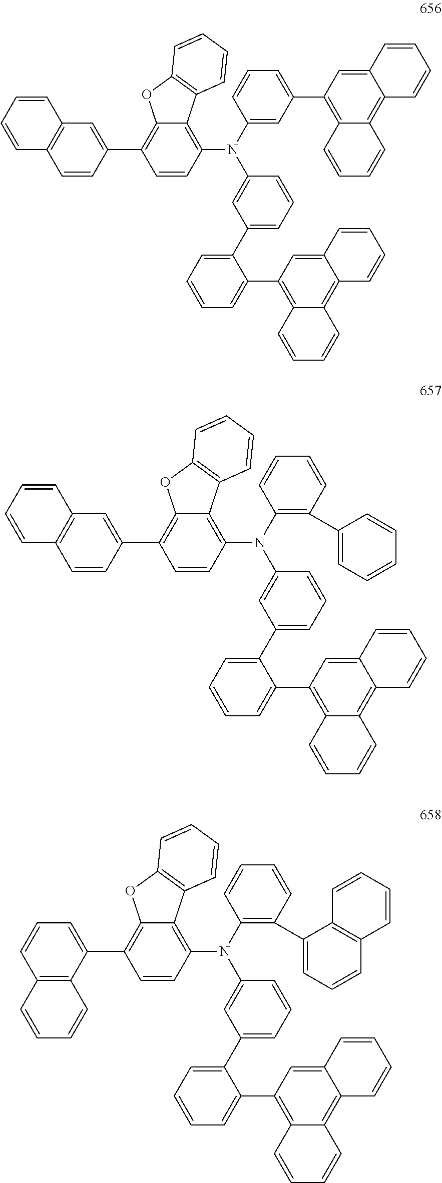 [SMILES: c1cc(-c2ccccc2-c2cc3ccccc3c3ccccc23)cc(N(c2cccc(-c3cc4ccccc4c4ccccc34)c2)c2ccc(-c3ccc4ccccc4c3)c3oc4ccccc4c23)c1 ; c1ccc(-c2ccccc2N(c2cccc(-c3ccccc3-c3cc4ccccc4c4ccccc34)c2)c2ccc(-c3ccc4ccccc4c3)c3oc4ccccc4c23)cc1 ; c1cc(-c2ccccc2-c2cc3ccccc3c3ccccc23)cc(N(c2ccccc2-c2cccc3ccccc23)c2ccc(-c3cccc4ccccc34)c3oc4ccccc4c23)c1]

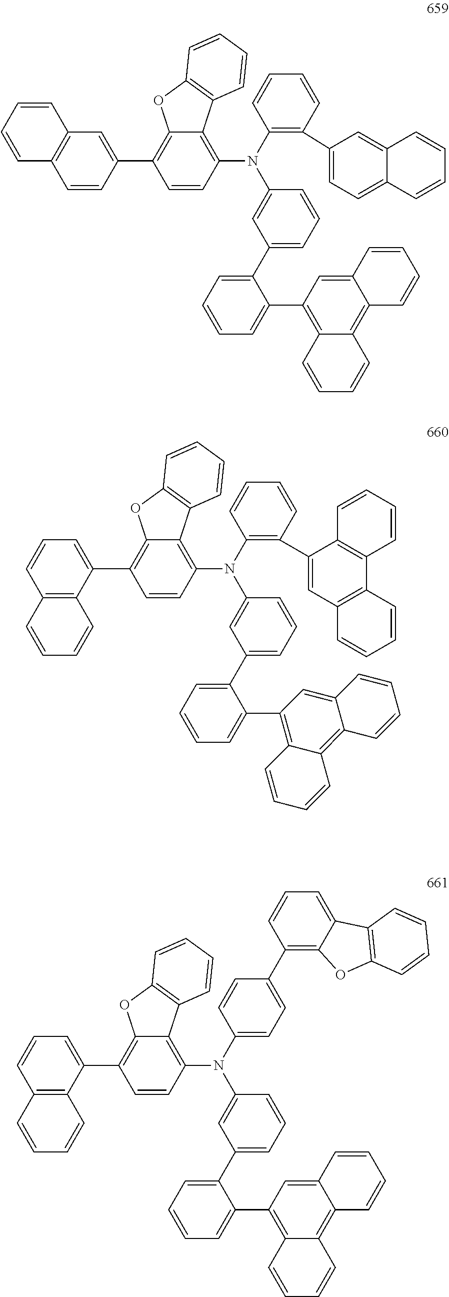 [SMILES: c1cc(-c2ccccc2-c2cc3ccccc3c3ccccc23)cc(N(c2ccccc2-c2ccc3ccccc3c2)c2ccc(-c3ccc4ccccc4c3)c3oc4ccccc4c23)c1 ; c1cc(-c2ccccc2-c2cc3ccccc3c3ccccc23)cc(N(c2ccccc2-c2cc3ccccc3c3ccccc23)c2ccc(-c3cccc4ccccc34)c3oc4ccccc4c23)c1 ; c1cc(-c2ccccc2-c2cc3ccccc3c3ccccc23)cc(N(c2ccc(-c3cccc4c3oc3ccccc34)cc2)c2ccc(-c3cccc4ccccc34)c3oc4ccccc4c23)c1]

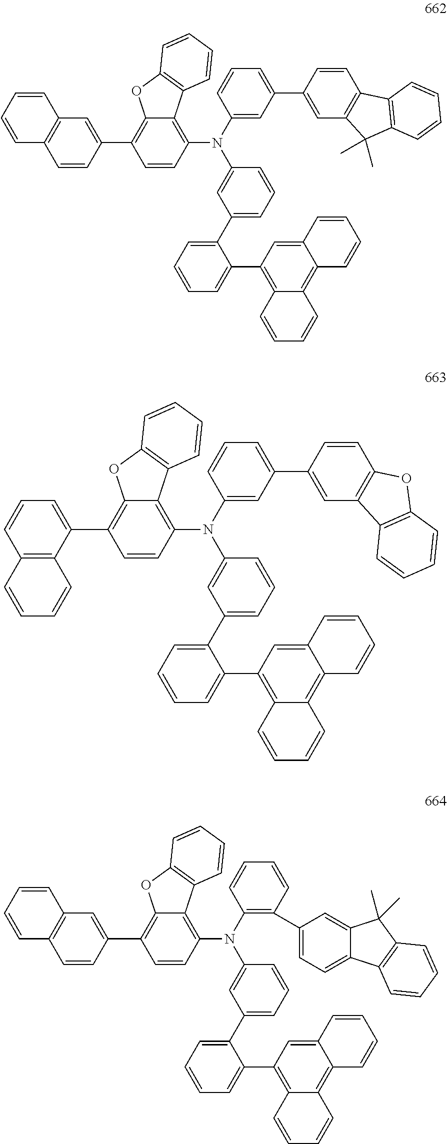 [SMILES: CC1(C)c2ccccc2-c2ccc(-c3cccc(N(c4cccc(-c5ccccc5-c5cc6ccccc6c6ccccc56)c4)c4ccc(-c5ccc6ccccc6c5)c5oc6ccccc6c45)c3)cc21 ; c1cc(-c2ccc3oc4ccccc4c3c2)cc(N(c2cccc(-c3ccccc3-c3cc4ccccc4c4ccccc34)c2)c2ccc(-c3cccc4ccccc34)c3oc4ccccc4c23)c1 ; CC1(C)c2ccccc2-c2ccc(-c3ccccc3N(c3cccc(-c4ccccc4-c4cc5ccccc5c5ccccc45)c3)c3ccc(-c4ccc5ccccc5c4)c4oc5ccccc5c34)cc21]

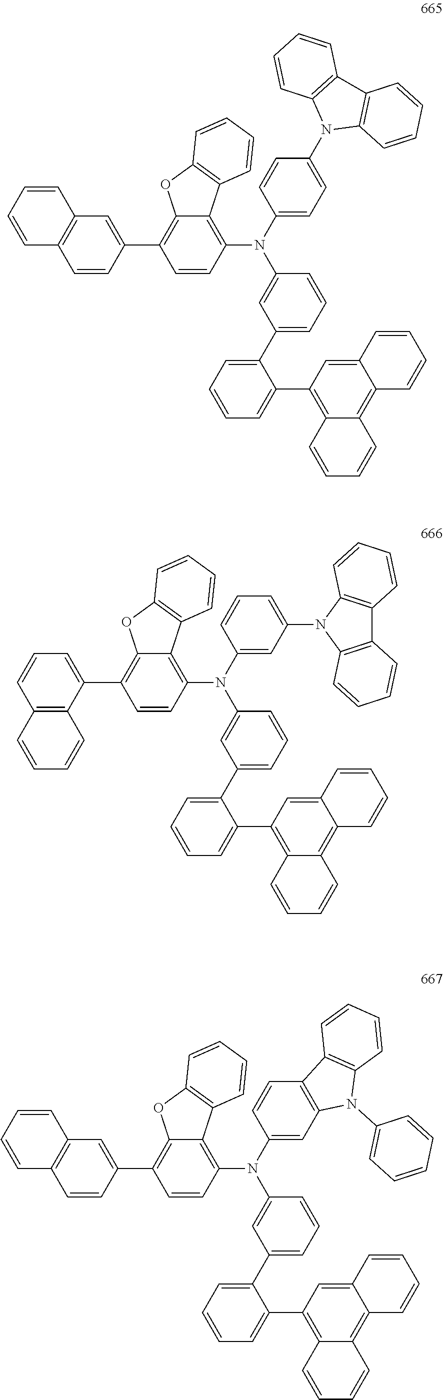 [SMILES: c1cc(-c2ccccc2-c2cc3ccccc3c3ccccc23)cc(N(c2ccc(-n3c4ccccc4c4ccccc43)cc2)c2ccc(-c3ccc4ccccc4c3)c3oc4ccccc4c23)c1 ; c1cc(-c2ccccc2-c2cc3ccccc3c3ccccc23)cc(N(c2cccc(-n3c4ccccc4c4ccccc43)c2)c2ccc(-c3cccc4ccccc34)c3oc4ccccc4c23)c1 ; c1ccc(-n2c3ccccc3c3ccc(N(c4cccc(-c5ccccc5-c5cc6ccccc6c6ccccc56)c4)c4ccc(-c5ccc6ccccc6c5)c5oc6ccccc6c45)cc32)cc1]

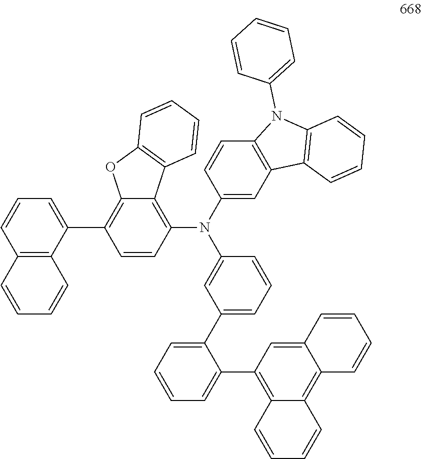 [SMILES: c1ccc(-n2c3ccccc3c3cc(N(c4cccc(-c5ccccc5-c5cc6ccccc6c6ccccc56)c4)c4ccc(-c5cccc6ccccc56)c5oc6ccccc6c45)ccc32)cc1]

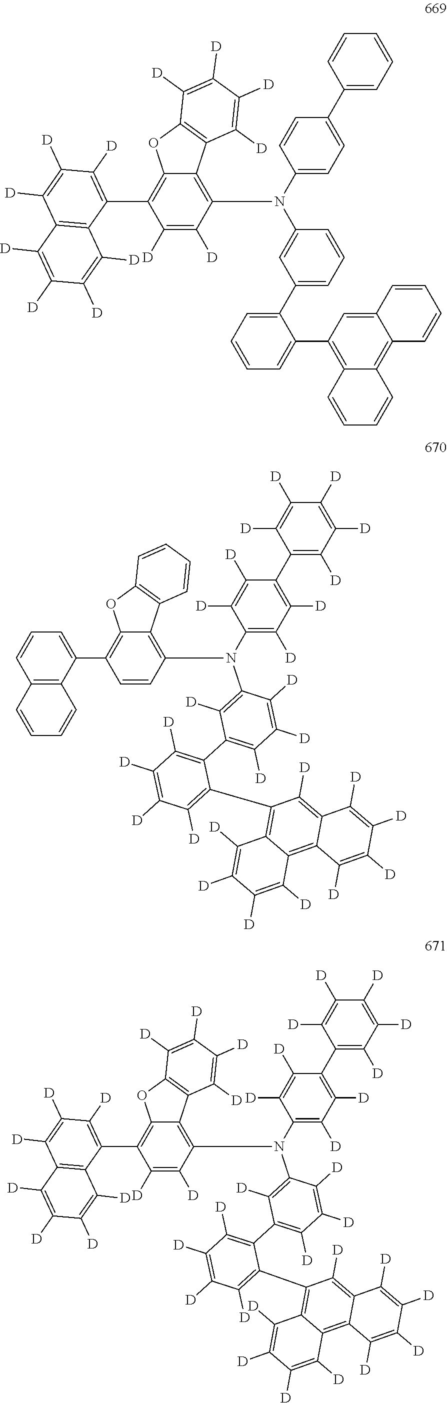

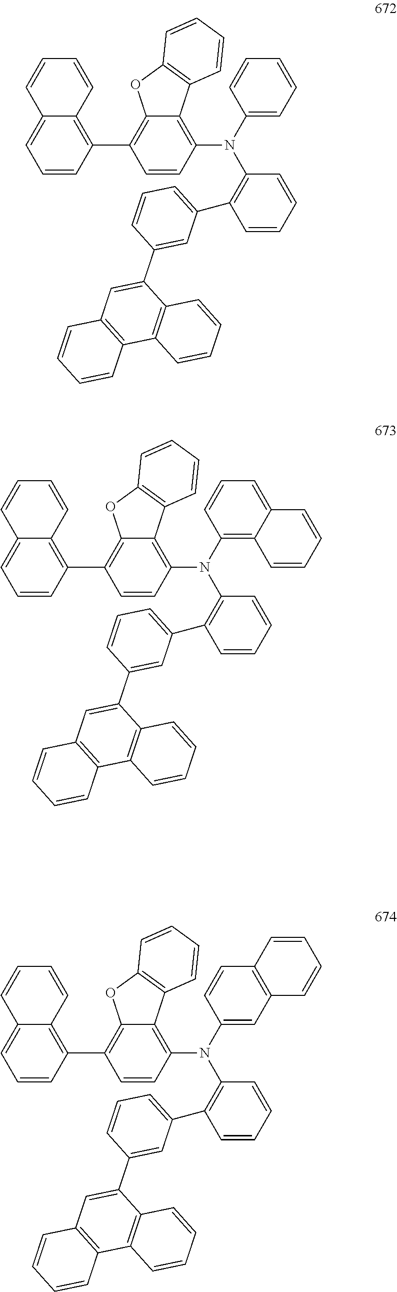 [SMILES: c1ccc(N(c2ccccc2-c2cccc(-c3cc4ccccc4c4ccccc34)c2)c2ccc(-c3cccc4ccccc34)c3oc4ccccc4c23)cc1 ; c1cc(-c2ccccc2N(c2cccc3ccccc23)c2ccc(-c3cccc4ccccc34)c3oc4ccccc4c23)cc(-c2cc3ccccc3c3ccccc23)c1 ; c1cc(-c2ccccc2N(c2ccc3ccccc3c2)c2ccc(-c3cccc4ccccc34)c3oc4ccccc4c23)cc(-c2cc3ccccc3c3ccccc23)c1]

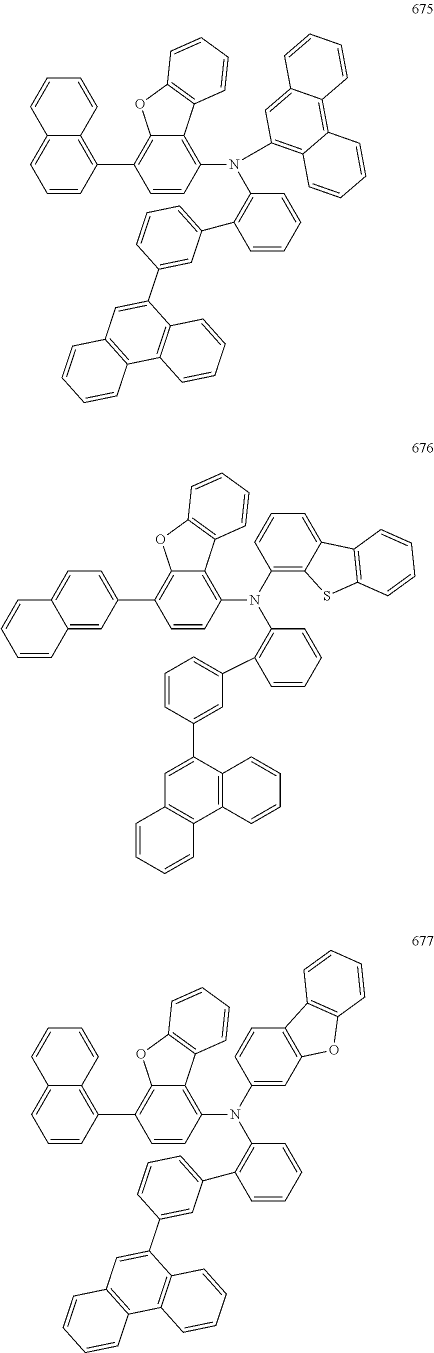 [SMILES: c1cc(-c2ccccc2N(c2cc3ccccc3c3ccccc23)c2ccc(-c3cccc4ccccc34)c3oc4ccccc4c23)cc(-c2cc3ccccc3c3ccccc23)c1 ; c1cc(-c2ccccc2N(c2cccc3c2sc2ccccc23)c2ccc(-c3ccc4ccccc4c3)c3oc4ccccc4c23)cc(-c2cc3ccccc3c3ccccc23)c1 ; c1cc(-c2ccccc2N(c2ccc3c(c2)oc2ccccc23)c2ccc(-c3cccc4ccccc34)c3oc4ccccc4c23)cc(-c2cc3ccccc3c3ccccc23)c1]

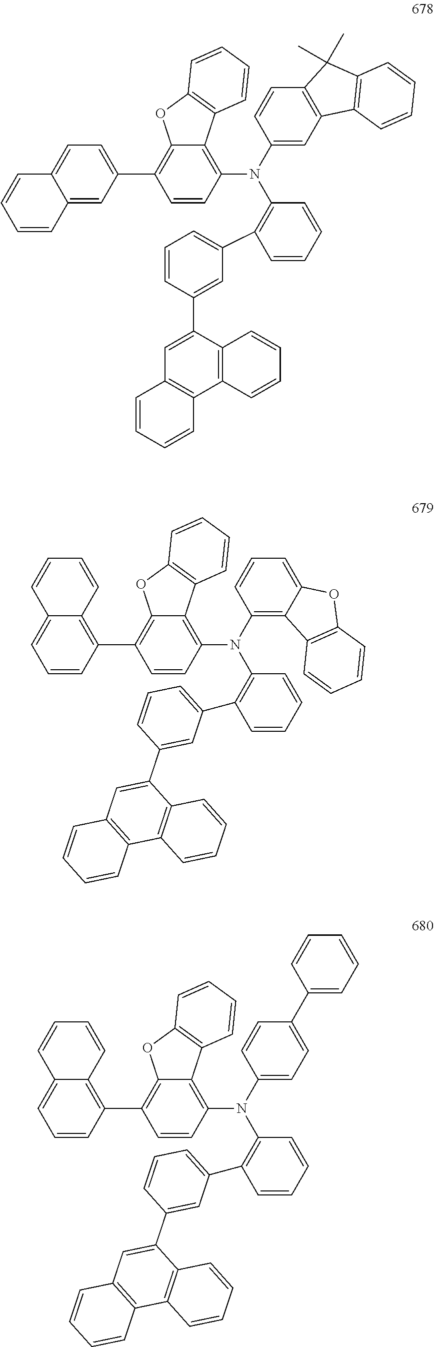 [SMILES: CC1(C)c2ccccc2-c2cc(N(c3ccccc3-c3cccc(-c4cc5ccccc5c5ccccc45)c3)c3ccc(-c4ccc5ccccc5c4)c4oc5ccccc5c34)ccc21 ; c1cc(-c2ccccc2N(c2cccc3oc4ccccc4c23)c2ccc(-c3cccc4ccccc34)c3oc4ccccc4c23)cc(-c2cc3ccccc3c3ccccc23)c1 ; c1ccc(-c2ccc(N(c3ccccc3-c3cccc(-c4cc5ccccc5c5ccccc45)c3)c3ccc(-c4cccc5ccccc45)c4oc5ccccc5c34)cc2)cc1]

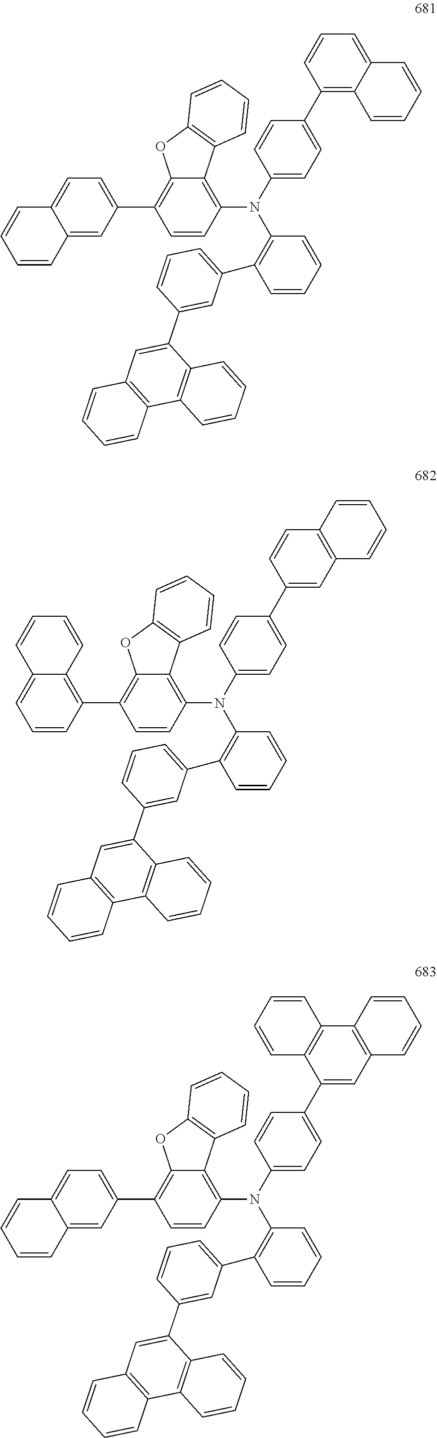 [SMILES: c1cc(-c2ccccc2N(c2ccc(-c3cccc4ccccc34)cc2)c2ccc(-c3ccc4ccccc4c3)c3oc4ccccc4c23)cc(-c2cc3ccccc3c3ccccc23)c1 ; c1cc(-c2ccccc2N(c2ccc(-c3ccc4ccccc4c3)cc2)c2ccc(-c3cccc4ccccc34)c3oc4ccccc4c23)cc(-c2cc3ccccc3c3ccccc23)c1 ; c1cc(-c2ccccc2N(c2ccc(-c3cc4ccccc4c4ccccc34)cc2)c2ccc(-c3ccc4ccccc4c3)c3oc4ccccc4c23)cc(-c2cc3ccccc3c3ccccc23)c1]

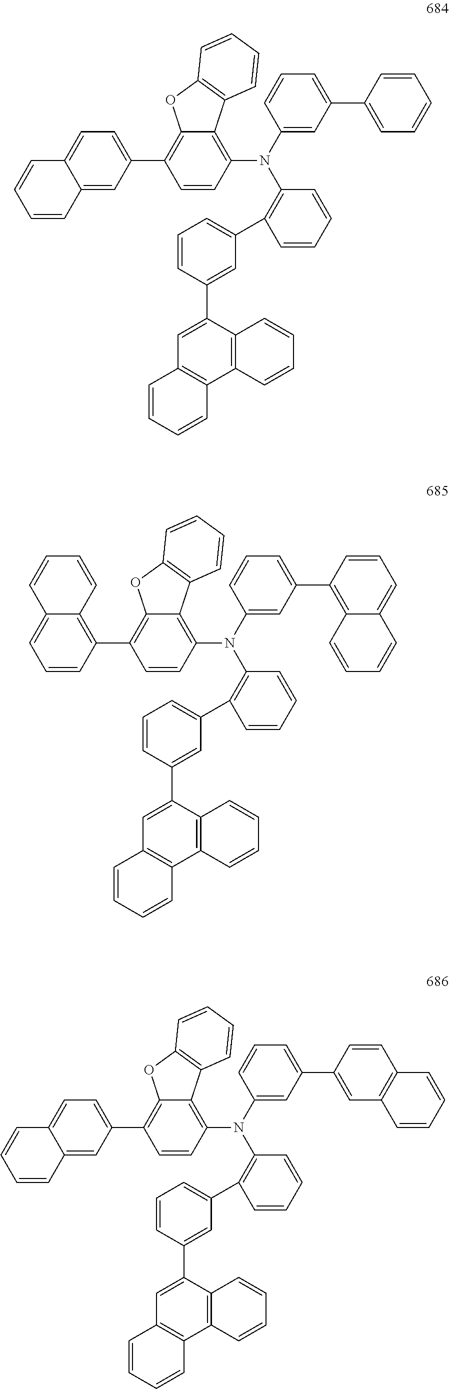 [SMILES: c1ccc(-c2cccc(N(c3ccccc3-c3cccc(-c4cc5ccccc5c5ccccc45)c3)c3ccc(-c4ccc5ccccc5c4)c4oc5ccccc5c34)c2)cc1 ; c1cc(-c2ccccc2N(c2cccc(-c3cccc4ccccc34)c2)c2ccc(-c3cccc4ccccc34)c3oc4ccccc4c23)cc(-c2cc3ccccc3c3ccccc23)c1 ; c1cc(-c2ccccc2N(c2cccc(-c3ccc4ccccc4c3)c2)c2ccc(-c3ccc4ccccc4c3)c3oc4ccccc4c23)cc(-c2cc3ccccc3c3ccccc23)c1]

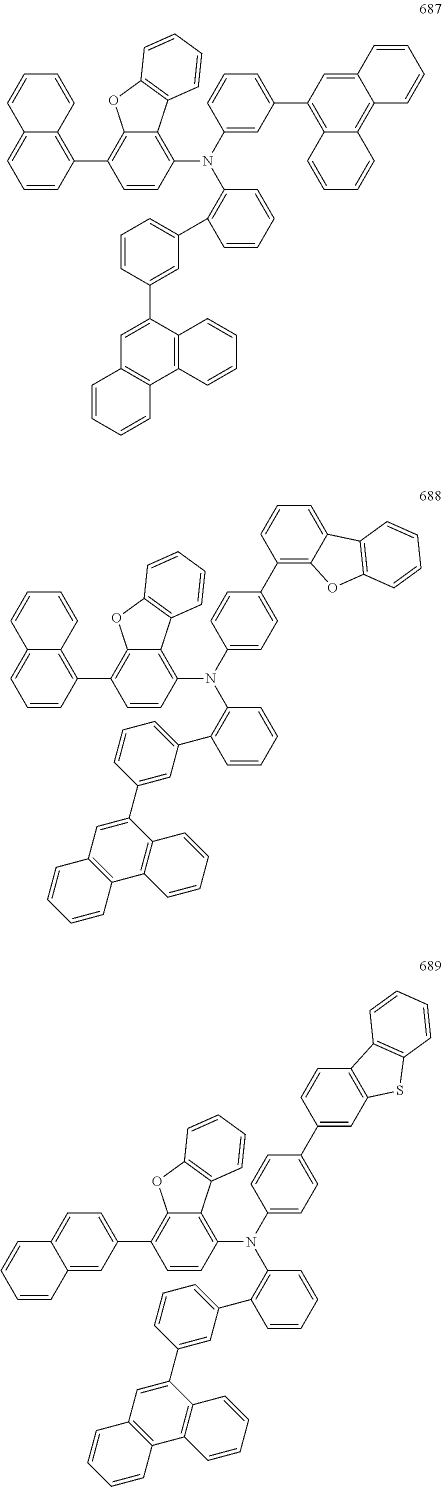 [SMILES: c1cc(-c2ccccc2N(c2cccc(-c3cc4ccccc4c4ccccc34)c2)c2ccc(-c3cccc4ccccc34)c3oc4ccccc4c23)cc(-c2cc3ccccc3c3ccccc23)c1 ; c1cc(-c2ccccc2N(c2ccc(-c3cccc4c3oc3ccccc34)cc2)c2ccc(-c3cccc4ccccc34)c3oc4ccccc4c23)cc(-c2cc3ccccc3c3ccccc23)c1 ; c1cc(-c2ccccc2N(c2ccc(-c3ccc4c(c3)sc3ccccc34)cc2)c2ccc(-c3ccc4ccccc4c3)c3oc4ccccc4c23)cc(-c2cc3ccccc3c3ccccc23)c1]

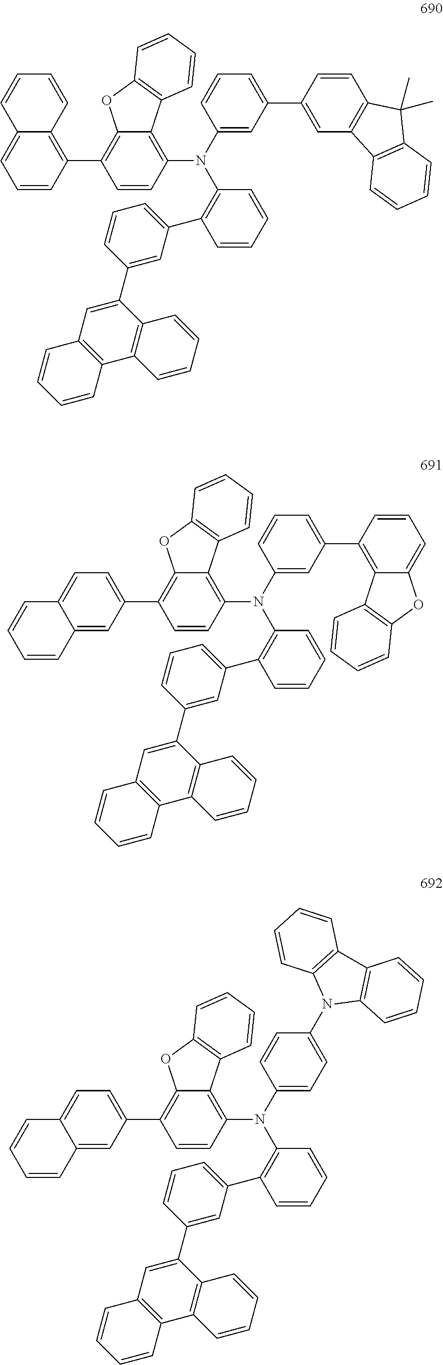 [SMILES: CC1(C)c2ccccc2-c2cc(-c3cccc(N(c4ccccc4-c4cccc(-c5cc6ccccc6c6ccccc56)c4)c4ccc(-c5cccc6ccccc56)c5oc6ccccc6c45)c3)ccc21 ; c1cc(-c2ccccc2N(c2cccc(-c3cccc4oc5ccccc5c34)c2)c2ccc(-c3ccc4ccccc4c3)c3oc4ccccc4c23)cc(-c2cc3ccccc3c3ccccc23)c1 ; c1cc(-c2ccccc2N(c2ccc(-n3c4ccccc4c4ccccc43)cc2)c2ccc(-c3ccc4ccccc4c3)c3oc4ccccc4c23)cc(-c2cc3ccccc3c3ccccc23)c1]

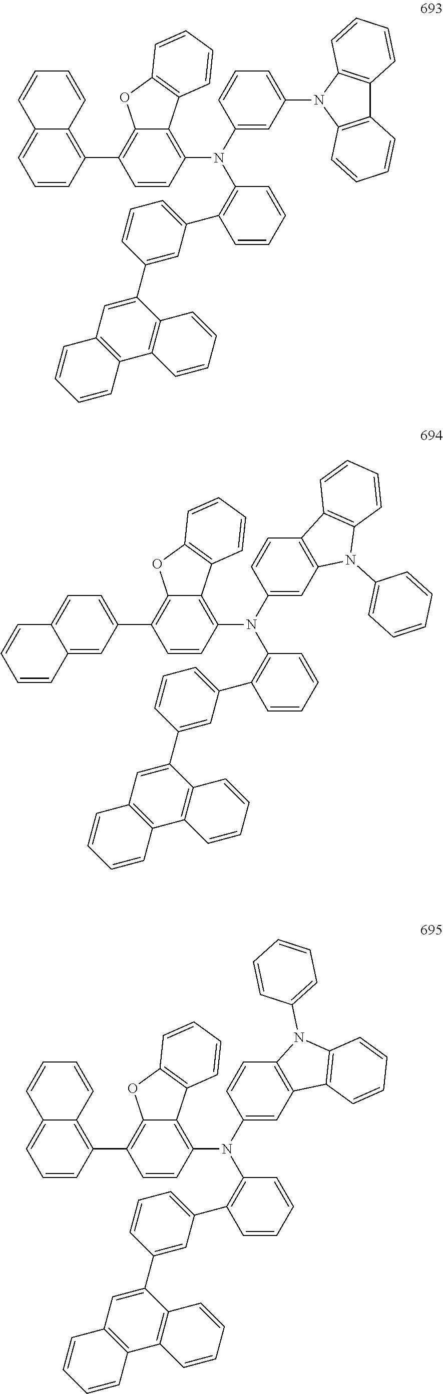 [SMILES: c1cc(-c2ccccc2N(c2cccc(-n3c4ccccc4c4ccccc43)c2)c2ccc(-c3cccc4ccccc34)c3oc4ccccc4c23)cc(-c2cc3ccccc3c3ccccc23)c1 ; c1ccc(-n2c3ccccc3c3ccc(N(c4ccccc4-c4cccc(-c5cc6ccccc6c6ccccc56)c4)c4ccc(-c5ccc6ccccc6c5)c5oc6ccccc6c45)cc32)cc1 ; c1ccc(-n2c3ccccc3c3cc(N(c4ccccc4-c4cccc(-c5cc6ccccc6c6ccccc56)c4)c4ccc(-c5cccc6ccccc56)c5oc6ccccc6c45)ccc32)cc1]

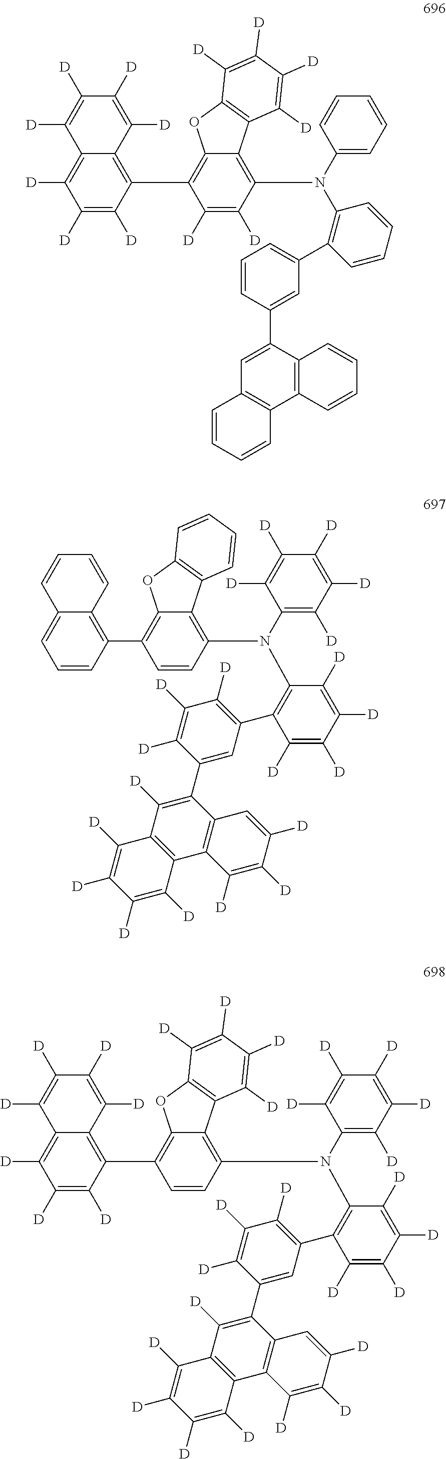

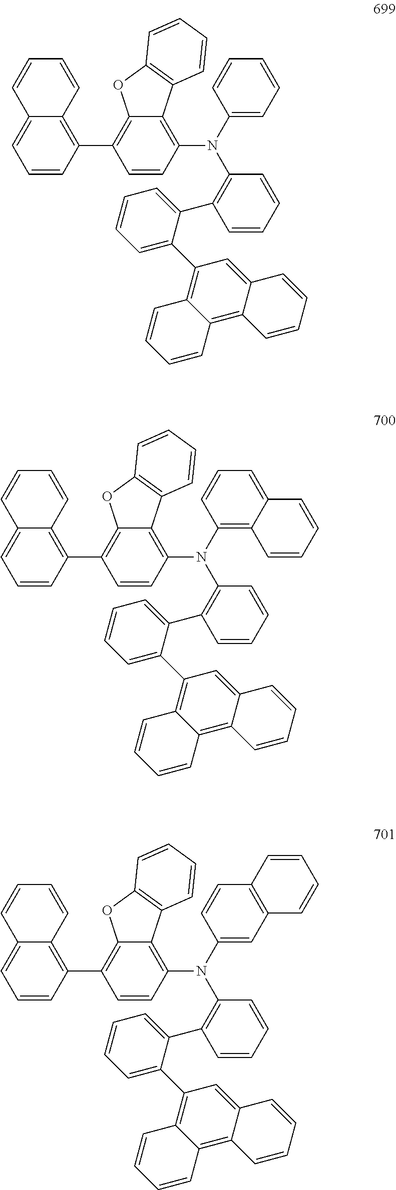 [SMILES: c1ccc(N(c2ccccc2-c2ccccc2-c2cc3ccccc3c3ccccc23)c2ccc(-c3cccc4ccccc34)c3oc4ccccc4c23)cc1 ; c1ccc(-c2cc3ccccc3c3ccccc23)c(-c2ccccc2N(c2cccc3ccccc23)c2ccc(-c3cccc4ccccc34)c3oc4ccccc4c23)c1 ; c1ccc(-c2cc3ccccc3c3ccccc23)c(-c2ccccc2N(c2ccc3ccccc3c2)c2ccc(-c3cccc4ccccc34)c3oc4ccccc4c23)c1]

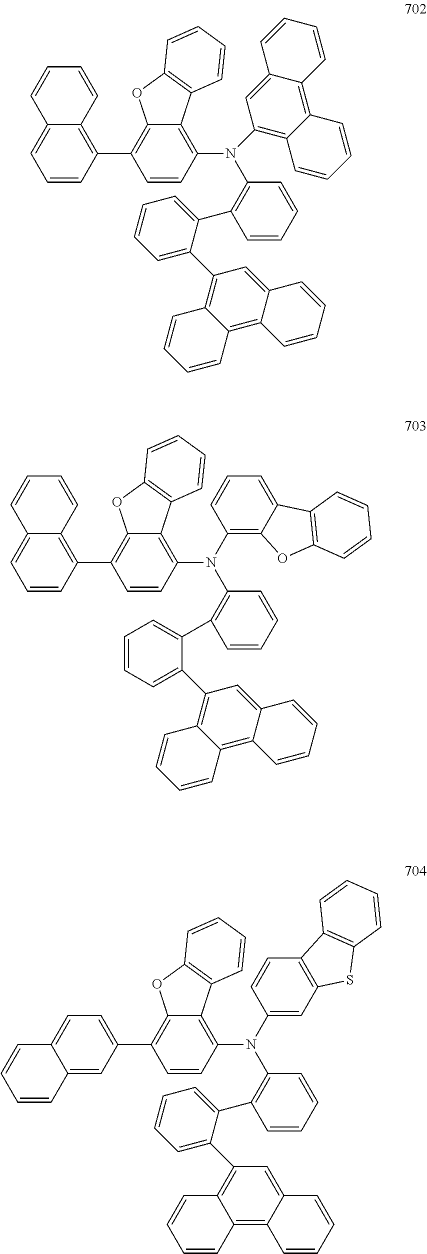 [SMILES: c1ccc(-c2cc3ccccc3c3ccccc23)c(-c2ccccc2N(c2cc3ccccc3c3ccccc23)c2ccc(-c3cccc4ccccc34)c3oc4ccccc4c23)c1 ; c1ccc(-c2cc3ccccc3c3ccccc23)c(-c2ccccc2N(c2cccc3c2oc2ccccc23)c2ccc(-c3cccc4ccccc34)c3oc4ccccc4c23)c1 ; c1ccc(-c2cc3ccccc3c3ccccc23)c(-c2ccccc2N(c2ccc3c(c2)sc2ccccc23)c2ccc(-c3ccc4ccccc4c3)c3oc4ccccc4c23)c1]

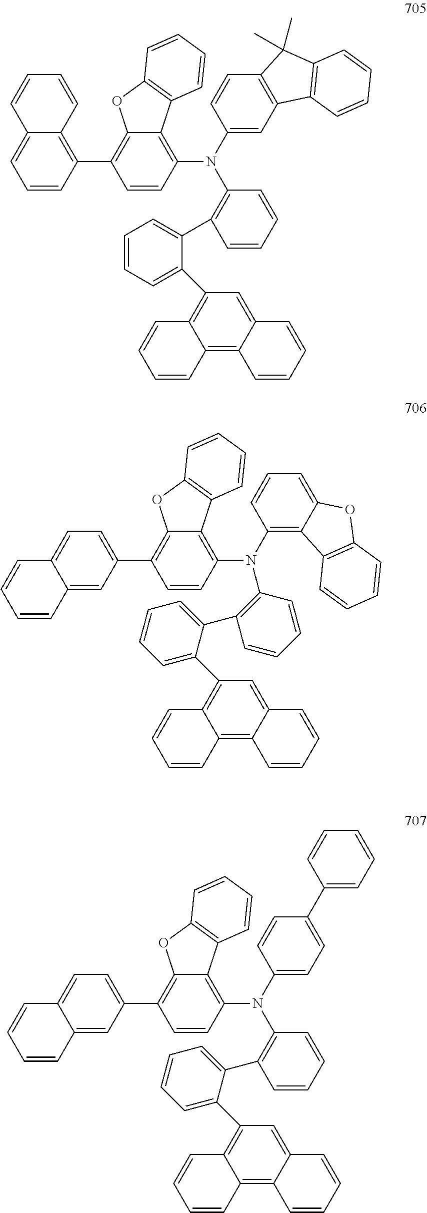 [SMILES: CC1(C)c2ccccc2-c2cc(N(c3ccccc3-c3ccccc3-c3cc4ccccc4c4ccccc34)c3ccc(-c4cccc5ccccc45)c4oc5ccccc5c34)ccc21 ; c1ccc(-c2cc3ccccc3c3ccccc23)c(-c2ccccc2N(c2cccc3oc4ccccc4c23)c2ccc(-c3ccc4ccccc4c3)c3oc4ccccc4c23)c1 ; c1ccc(-c2ccc(N(c3ccccc3-c3ccccc3-c3cc4ccccc4c4ccccc34)c3ccc(-c4ccc5ccccc5c4)c4oc5ccccc5c34)cc2)cc1]

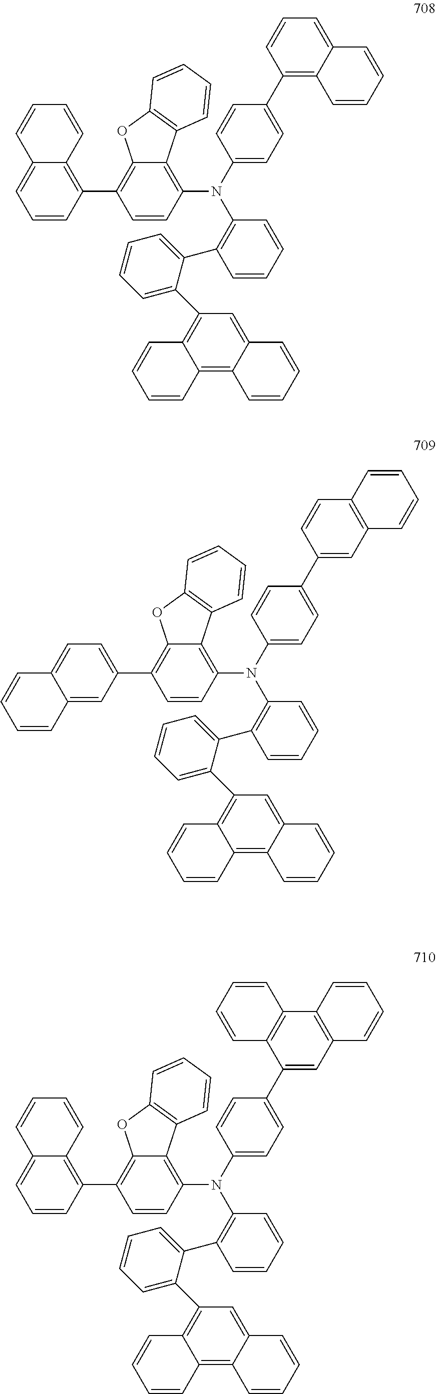 [SMILES: c1ccc(-c2cc3ccccc3c3ccccc23)c(-c2ccccc2N(c2ccc(-c3cccc4ccccc34)cc2)c2ccc(-c3cccc4ccccc34)c3oc4ccccc4c23)c1 ; c1ccc(-c2cc3ccccc3c3ccccc23)c(-c2ccccc2N(c2ccc(-c3ccc4ccccc4c3)cc2)c2ccc(-c3ccc4ccccc4c3)c3oc4ccccc4c23)c1 ; c1ccc(-c2cc3ccccc3c3ccccc23)c(-c2ccccc2N(c2ccc(-c3cc4ccccc4c4ccccc34)cc2)c2ccc(-c3cccc4ccccc34)c3oc4ccccc4c23)c1]

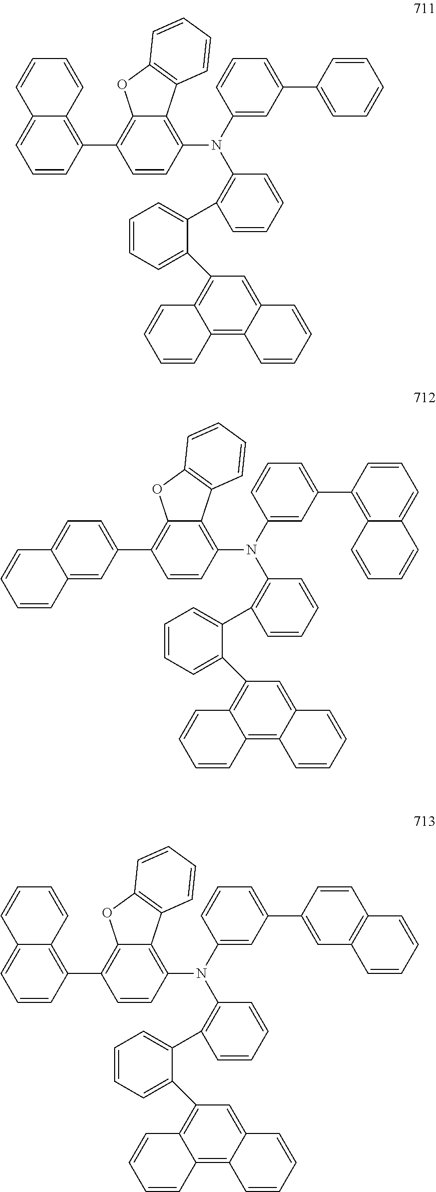 [SMILES: c1ccc(-c2cccc(N(c3ccccc3-c3ccccc3-c3cc4ccccc4c4ccccc34)c3ccc(-c4cccc5ccccc45)c4oc5ccccc5c34)c2)cc1 ; c1cc(-c2cccc3ccccc23)cc(N(c2ccccc2-c2ccccc2-c2cc3ccccc3c3ccccc23)c2ccc(-c3ccc4ccccc4c3)c3oc4ccccc4c23)c1 ; c1cc(-c2ccc3ccccc3c2)cc(N(c2ccccc2-c2ccccc2-c2cc3ccccc3c3ccccc23)c2ccc(-c3cccc4ccccc34)c3oc4ccccc4c23)c1]

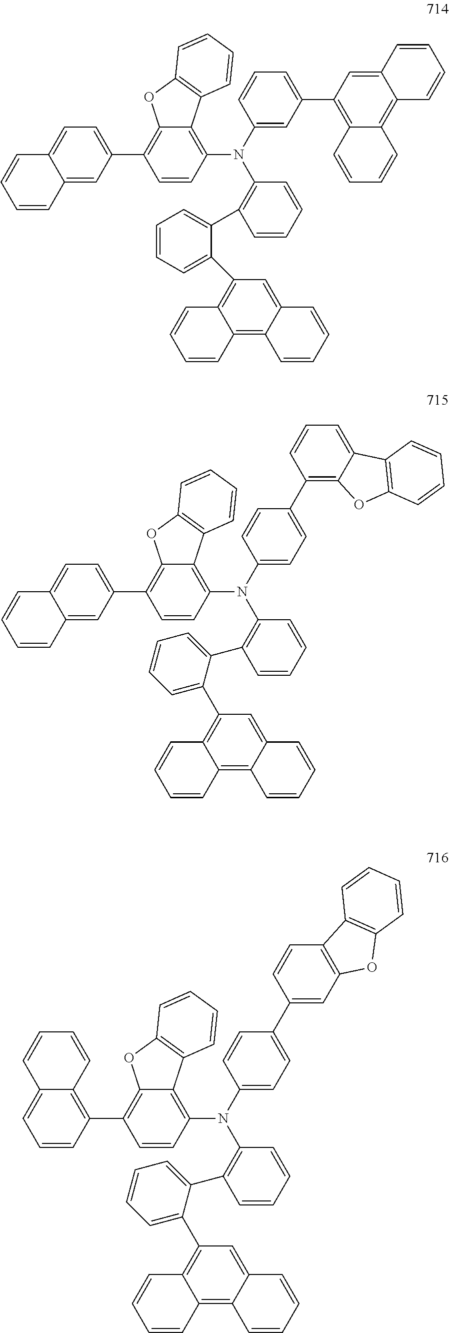 [SMILES: c1cc(-c2cc3ccccc3c3ccccc23)cc(N(c2ccccc2-c2ccccc2-c2cc3ccccc3c3ccccc23)c2ccc(-c3ccc4ccccc4c3)c3oc4ccccc4c23)c1 ; c1ccc(-c2cc3ccccc3c3ccccc23)c(-c2ccccc2N(c2ccc(-c3cccc4c3oc3ccccc34)cc2)c2ccc(-c3ccc4ccccc4c3)c3oc4ccccc4c23)c1 ; c1ccc(-c2cc3ccccc3c3ccccc23)c(-c2ccccc2N(c2ccc(-c3ccc4c(c3)oc3ccccc34)cc2)c2ccc(-c3cccc4ccccc34)c3oc4ccccc4c23)c1]

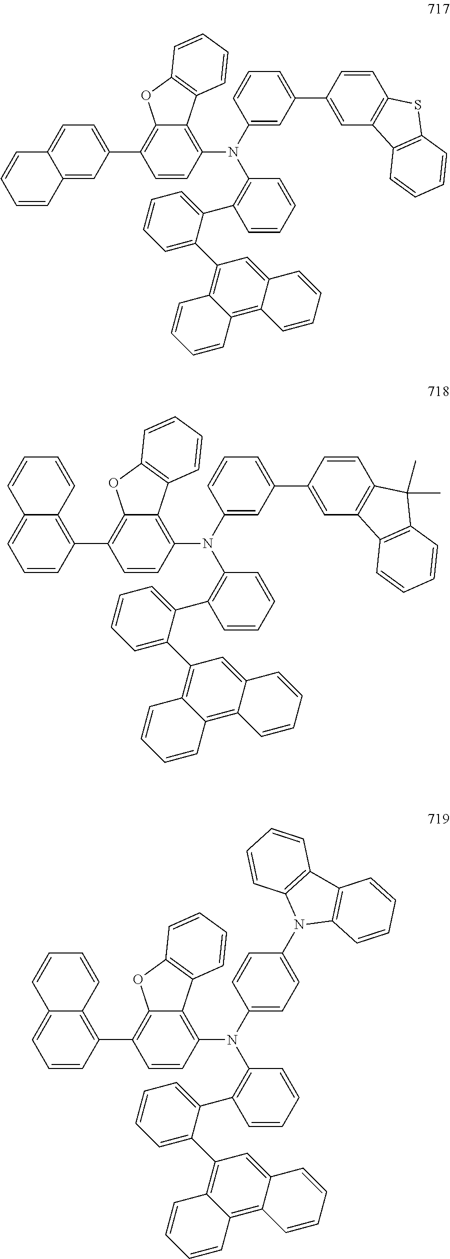 [SMILES: c1cc(-c2ccc3sc4ccccc4c3c2)cc(N(c2ccccc2-c2ccccc2-c2cc3ccccc3c3ccccc23)c2ccc(-c3ccc4ccccc4c3)c3oc4ccccc4c23)c1 ; CC1(C)c2ccccc2-c2cc(-c3cccc(N(c4ccccc4-c4ccccc4-c4cc5ccccc5c5ccccc45)c4ccc(-c5cccc6ccccc56)c5oc6ccccc6c45)c3)ccc21 ; c1ccc(-c2cc3ccccc3c3ccccc23)c(-c2ccccc2N(c2ccc(-n3c4ccccc4c4ccccc43)cc2)c2ccc(-c3cccc4ccccc34)c3oc4ccccc4c23)c1]

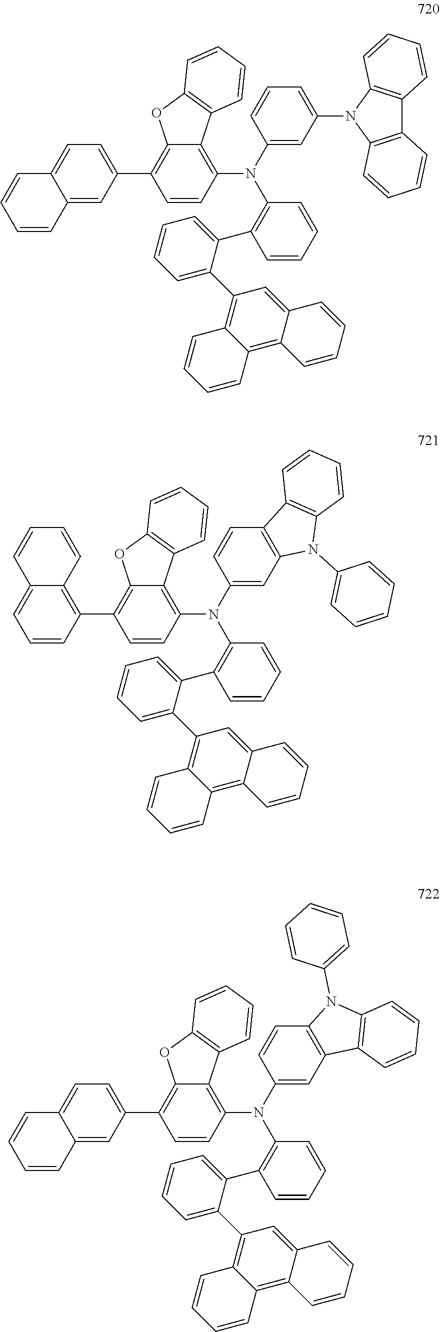 [SMILES: c1cc(N(c2ccccc2-c2ccccc2-c2cc3ccccc3c3ccccc23)c2ccc(-c3ccc4ccccc4c3)c3oc4ccccc4c23)cc(-n2c3ccccc3c3ccccc32)c1 ; c1ccc(-n2c3ccccc3c3ccc(N(c4ccccc4-c4ccccc4-c4cc5ccccc5c5ccccc45)c4ccc(-c5cccc6ccccc56)c5oc6ccccc6c45)cc32)cc1 ; c1ccc(-n2c3ccccc3c3cc(N(c4ccccc4-c4ccccc4-c4cc5ccccc5c5ccccc45)c4ccc(-c5ccc6ccccc6c5)c5oc6ccccc6c45)ccc32)cc1]

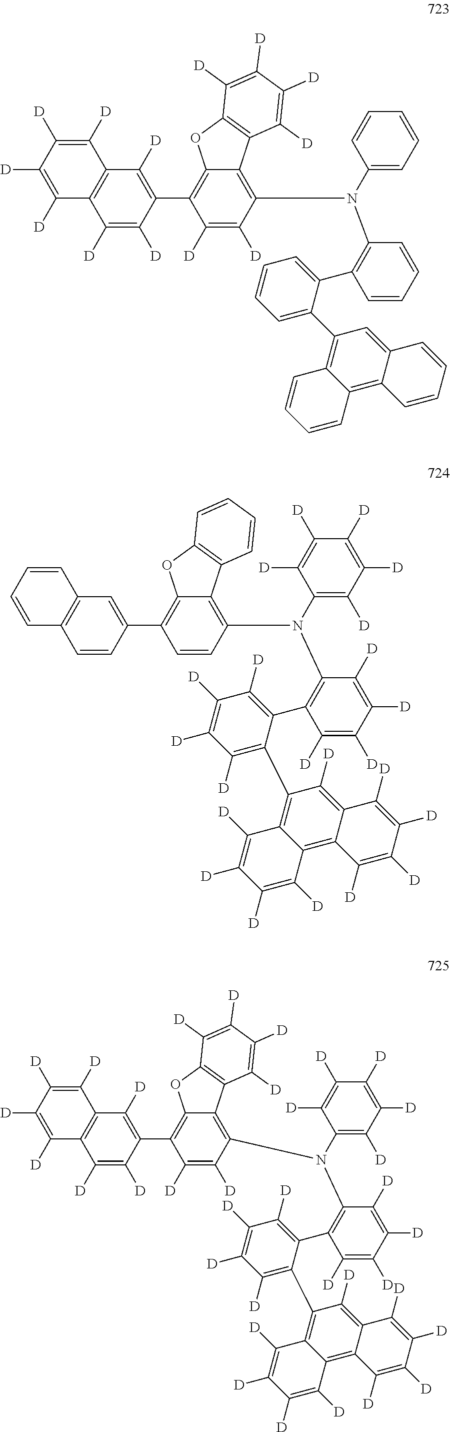

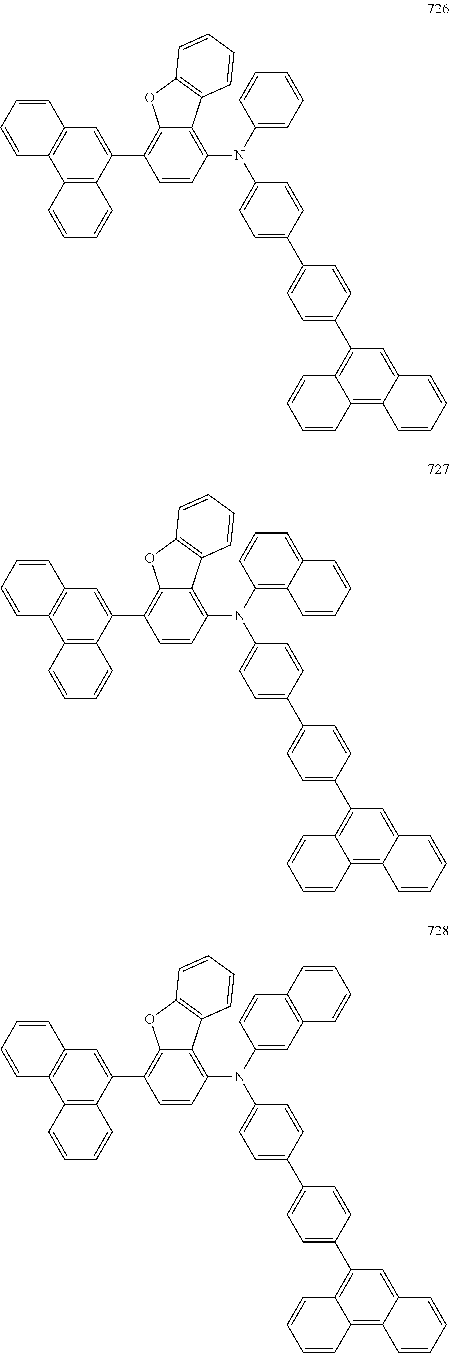 [SMILES: c1ccc(N(c2ccc(-c3ccc(-c4cc5ccccc5c5ccccc45)cc3)cc2)c2ccc(-c3cc4ccccc4c4ccccc34)c3oc4ccccc4c23)cc1 ; c1ccc2c(N(c3ccc(-c4ccc(-c5cc6ccccc6c6ccccc56)cc4)cc3)c3ccc(-c4cc5ccccc5c5ccccc45)c4oc5ccccc5c34)cccc2c1 ; c1ccc2cc(N(c3ccc(-c4ccc(-c5cc6ccccc6c6ccccc56)cc4)cc3)c3ccc(-c4cc5ccccc5c5ccccc45)c4oc5ccccc5c34)ccc2c1]

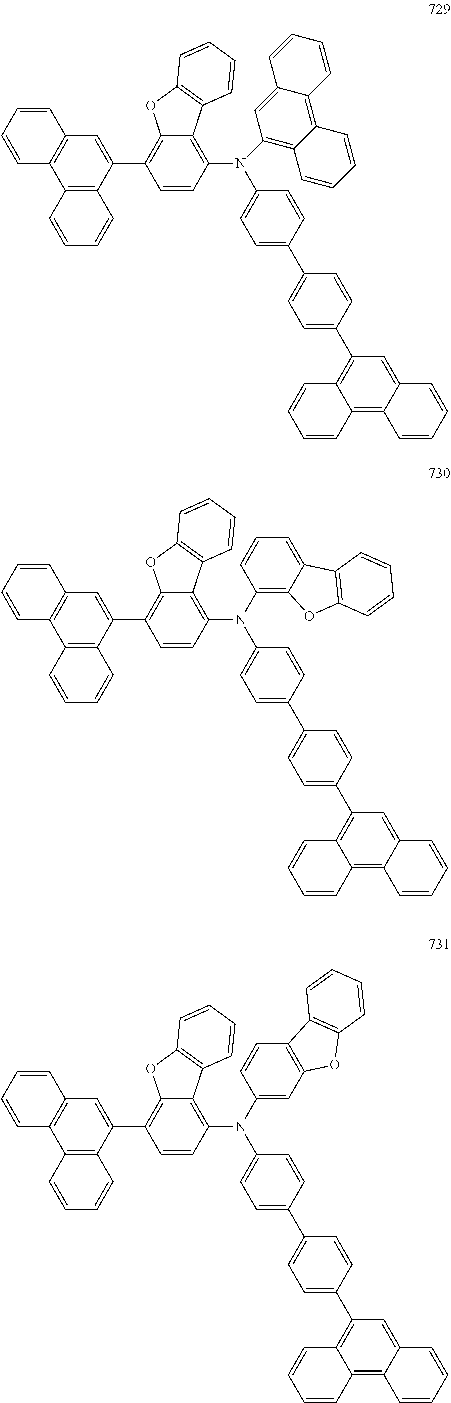 [SMILES: c1ccc2c(c1)cc(-c1ccc(-c3ccc(N(c4cc5ccccc5c5ccccc45)c4ccc(-c5cc6ccccc6c6ccccc56)c5oc6ccccc6c45)cc3)cc1)c1ccccc12 ; c1ccc2c(c1)cc(-c1ccc(-c3ccc(N(c4cccc5c4oc4ccccc45)c4ccc(-c5cc6ccccc6c6ccccc56)c5oc6ccccc6c45)cc3)cc1)c1ccccc12 ; c1ccc2c(c1)cc(-c1ccc(-c3ccc(N(c4ccc5c(c4)oc4ccccc45)c4ccc(-c5cc6ccccc6c6ccccc56)c5oc6ccccc6c45)cc3)cc1)c1ccccc12]

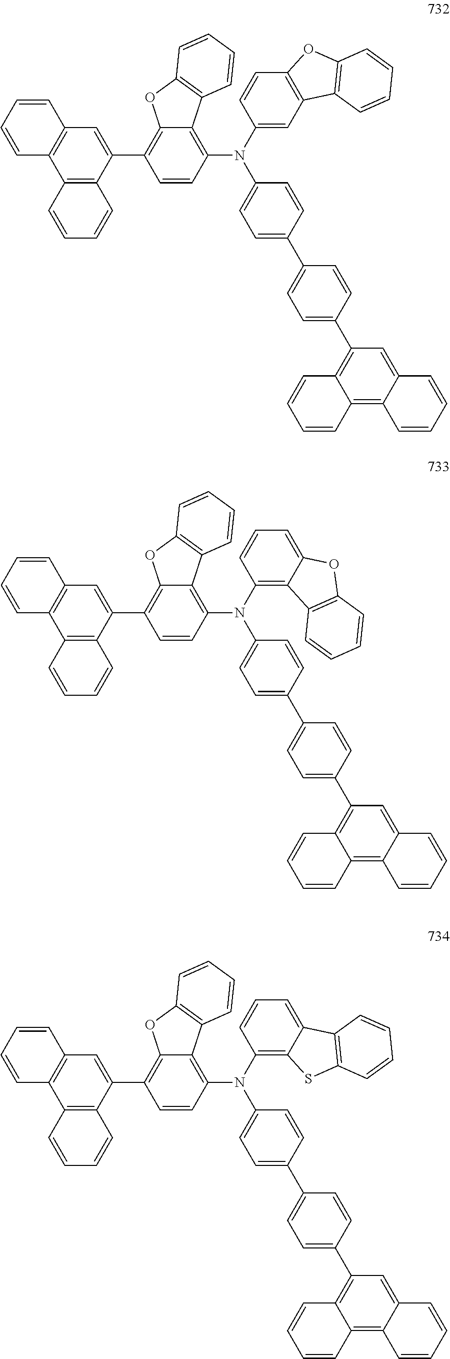 [SMILES: c1ccc2c(c1)cc(-c1ccc(-c3ccc(N(c4ccc5oc6ccccc6c5c4)c4ccc(-c5cc6ccccc6c6ccccc56)c5oc6ccccc6c45)cc3)cc1)c1ccccc12 ; c1ccc2c(c1)cc(-c1ccc(-c3ccc(N(c4cccc5oc6ccccc6c45)c4ccc(-c5cc6ccccc6c6ccccc56)c5oc6ccccc6c45)cc3)cc1)c1ccccc12 ; c1ccc2c(c1)cc(-c1ccc(-c3ccc(N(c4cccc5c4sc4ccccc45)c4ccc(-c5cc6ccccc6c6ccccc56)c5oc6ccccc6c45)cc3)cc1)c1ccccc12]

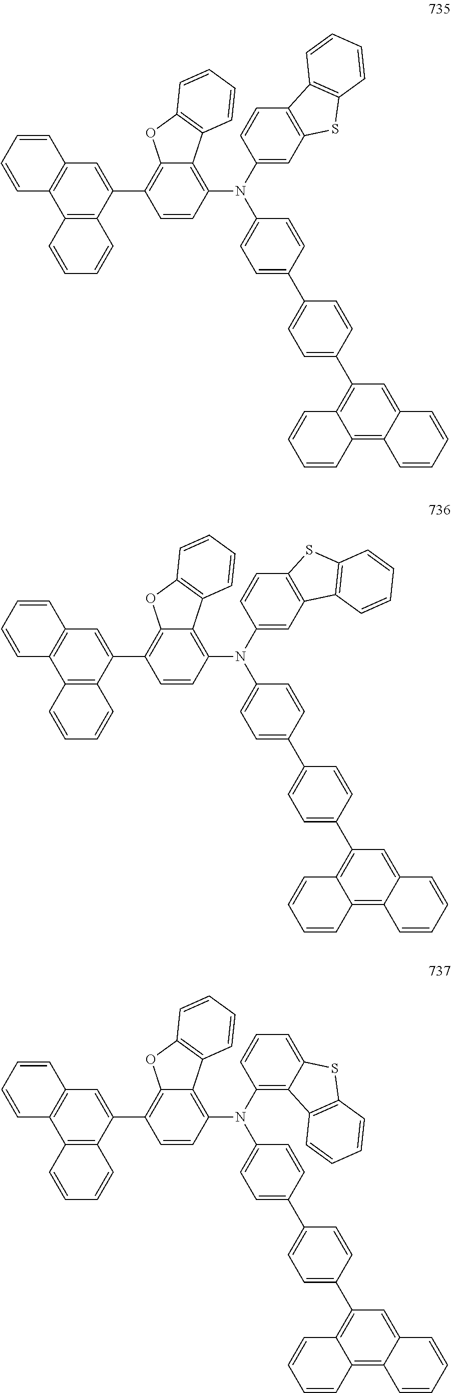 [SMILES: c1ccc2c(c1)cc(-c1ccc(-c3ccc(N(c4ccc5c(c4)sc4ccccc45)c4ccc(-c5cc6ccccc6c6ccccc56)c5oc6ccccc6c45)cc3)cc1)c1ccccc12 ; c1ccc2c(c1)cc(-c1ccc(-c3ccc(N(c4ccc5sc6ccccc6c5c4)c4ccc(-c5cc6ccccc6c6ccccc56)c5oc6ccccc6c45)cc3)cc1)c1ccccc12 ; c1ccc2c(c1)cc(-c1ccc(-c3ccc(N(c4cccc5sc6ccccc6c45)c4ccc(-c5cc6ccccc6c6ccccc56)c5oc6ccccc6c45)cc3)cc1)c1ccccc12]

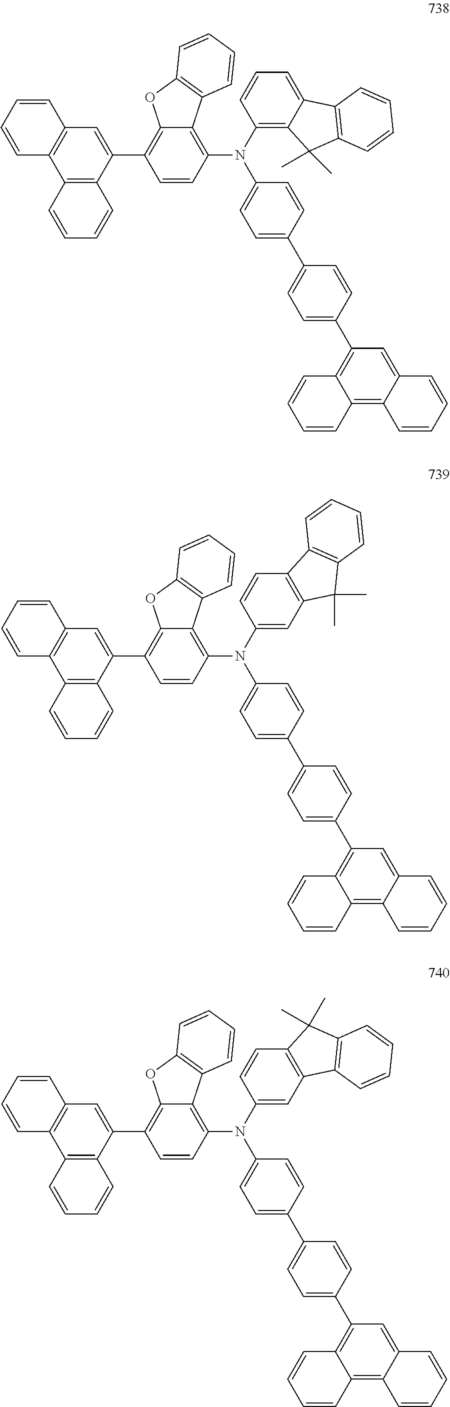 [SMILES: CC1(C)c2ccccc2-c2cccc(N(c3ccc(-c4ccc(-c5cc6ccccc6c6ccccc56)cc4)cc3)c3ccc(-c4cc5ccccc5c5ccccc45)c4oc5ccccc5c34)c21 ; CC1(C)c2ccccc2-c2ccc(N(c3ccc(-c4ccc(-c5cc6ccccc6c6ccccc56)cc4)cc3)c3ccc(-c4cc5ccccc5c5ccccc45)c4oc5ccccc5c34)cc21 ; CC1(C)c2ccccc2-c2cc(N(c3ccc(-c4ccc(-c5cc6ccccc6c6ccccc56)cc4)cc3)c3ccc(-c4cc5ccccc5c5ccccc45)c4oc5ccccc5c34)ccc21]

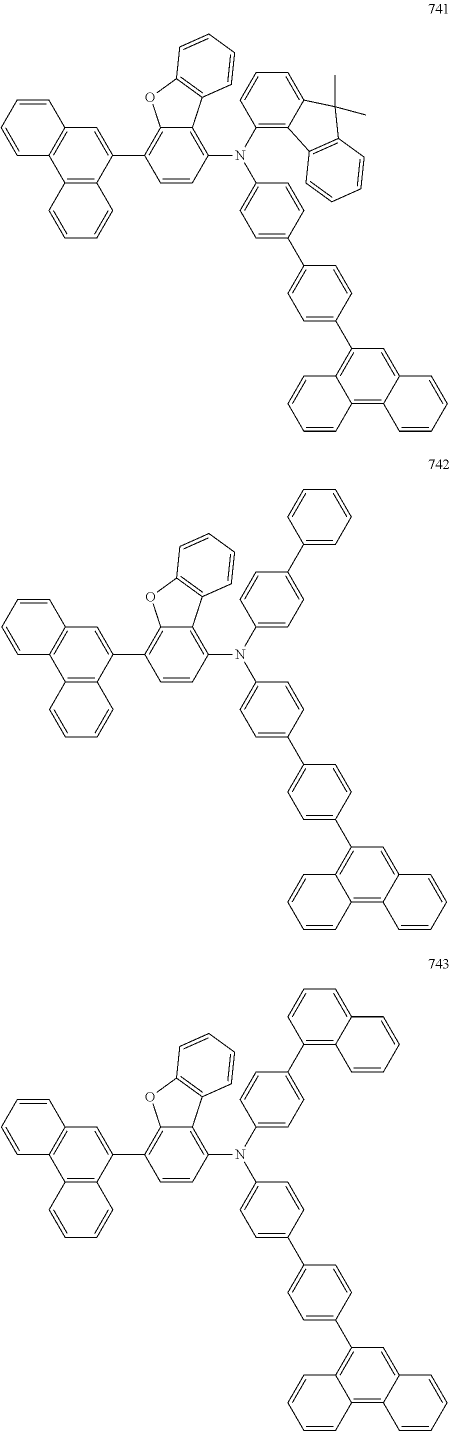 [SMILES: CC1(C)c2ccccc2-c2c(N(c3ccc(-c4ccc(-c5cc6ccccc6c6ccccc56)cc4)cc3)c3ccc(-c4cc5ccccc5c5ccccc45)c4oc5ccccc5c34)cccc21 ; c1ccc(-c2ccc(N(c3ccc(-c4ccc(-c5cc6ccccc6c6ccccc56)cc4)cc3)c3ccc(-c4cc5ccccc5c5ccccc45)c4oc5ccccc5c34)cc2)cc1 ; c1ccc2c(-c3ccc(N(c4ccc(-c5ccc(-c6cc7ccccc7c7ccccc67)cc5)cc4)c4ccc(-c5cc6ccccc6c6ccccc56)c5oc6ccccc6c45)cc3)cccc2c1]

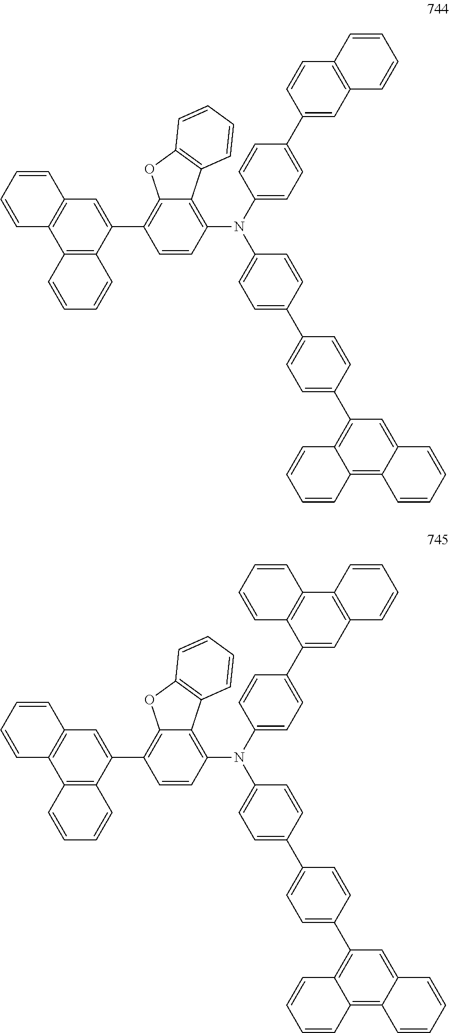 [SMILES: c1ccc2cc(-c3ccc(N(c4ccc(-c5ccc(-c6cc7ccccc7c7ccccc67)cc5)cc4)c4ccc(-c5cc6ccccc6c6ccccc56)c5oc6ccccc6c45)cc3)ccc2c1 ; c1ccc2c(c1)cc(-c1ccc(-c3ccc(N(c4ccc(-c5cc6ccccc6c6ccccc56)cc4)c4ccc(-c5cc6ccccc6c6ccccc56)c5oc6ccccc6c45)cc3)cc1)c1ccccc12]

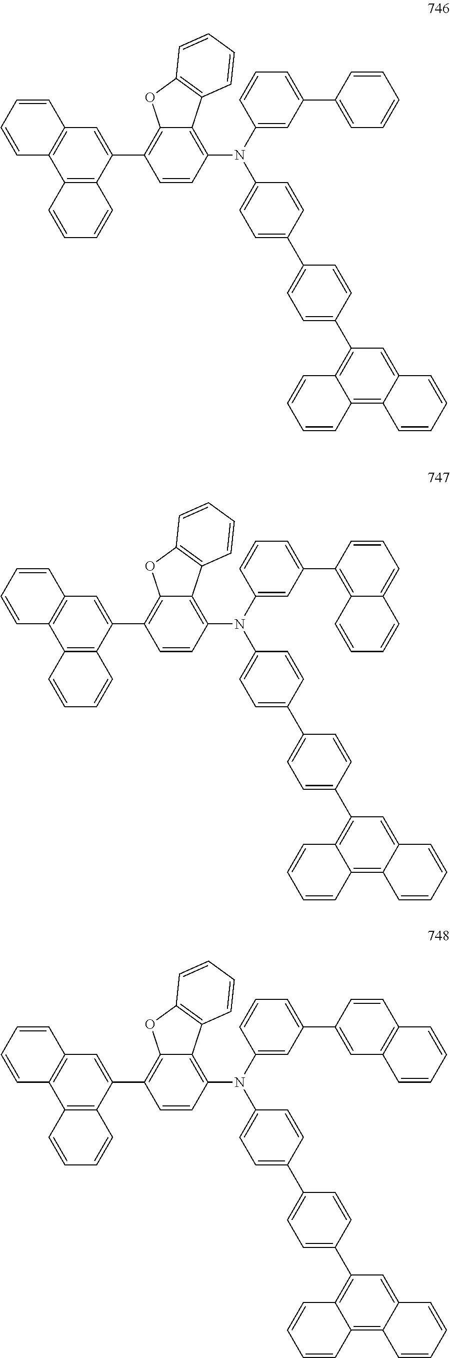 [SMILES: c1ccc(-c2cccc(N(c3ccc(-c4ccc(-c5cc6ccccc6c6ccccc56)cc4)cc3)c3ccc(-c4cc5ccccc5c5ccccc45)c4oc5ccccc5c34)c2)cc1 ; c1cc(-c2cccc3ccccc23)cc(N(c2ccc(-c3ccc(-c4cc5ccccc5c5ccccc45)cc3)cc2)c2ccc(-c3cc4ccccc4c4ccccc34)c3oc4ccccc4c23)c1 ; c1cc(-c2ccc3ccccc3c2)cc(N(c2ccc(-c3ccc(-c4cc5ccccc5c5ccccc45)cc3)cc2)c2ccc(-c3cc4ccccc4c4ccccc34)c3oc4ccccc4c23)c1]

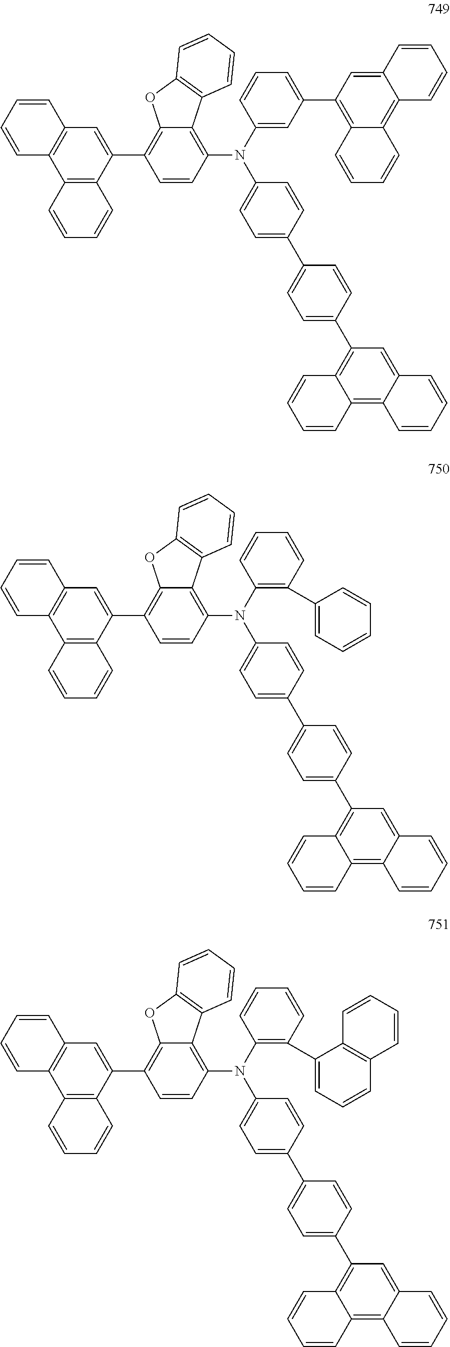 [SMILES: c1cc(-c2cc3ccccc3c3ccccc23)cc(N(c2ccc(-c3ccc(-c4cc5ccccc5c5ccccc45)cc3)cc2)c2ccc(-c3cc4ccccc4c4ccccc34)c3oc4ccccc4c23)c1 ; c1ccc(-c2ccccc2N(c2ccc(-c3ccc(-c4cc5ccccc5c5ccccc45)cc3)cc2)c2ccc(-c3cc4ccccc4c4ccccc34)c3oc4ccccc4c23)cc1 ; c1ccc(N(c2ccc(-c3ccc(-c4cc5ccccc5c5ccccc45)cc3)cc2)c2ccc(-c3cc4ccccc4c4ccccc34)c3oc4ccccc4c23)c(-c2cccc3ccccc23)c1]

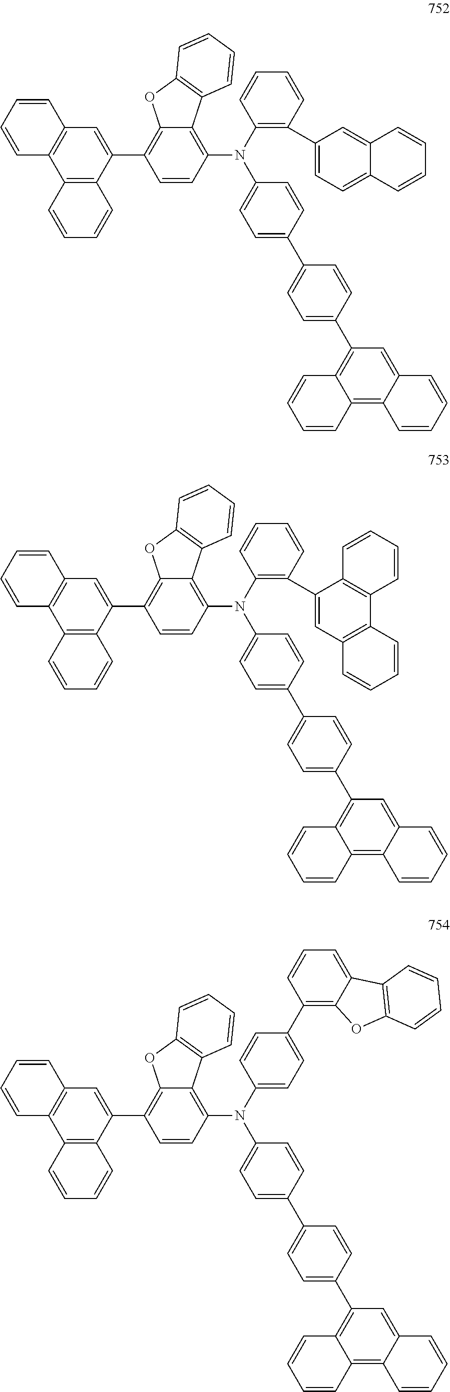

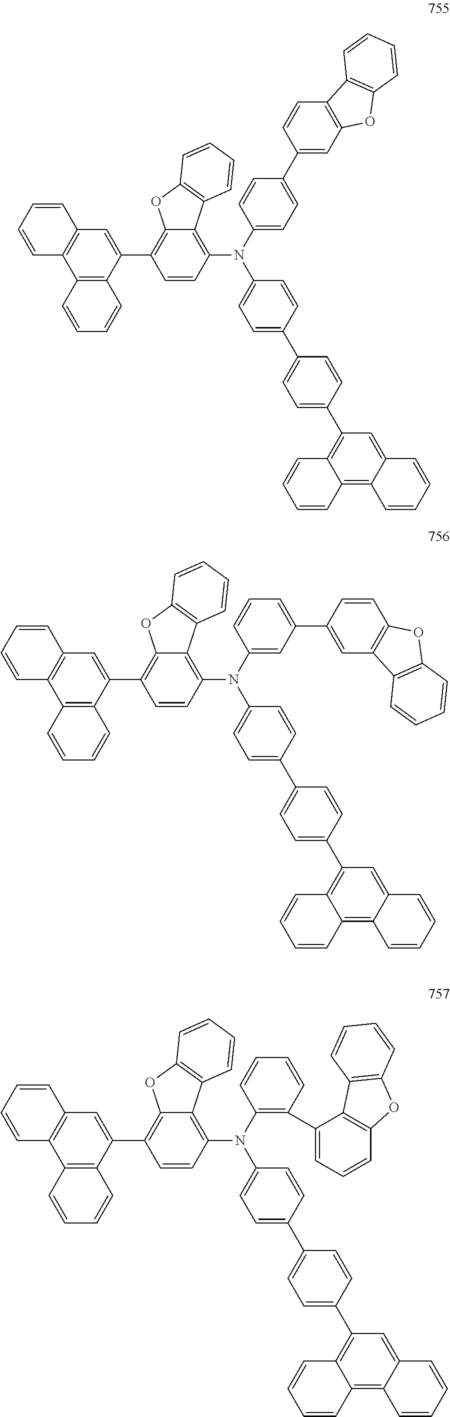

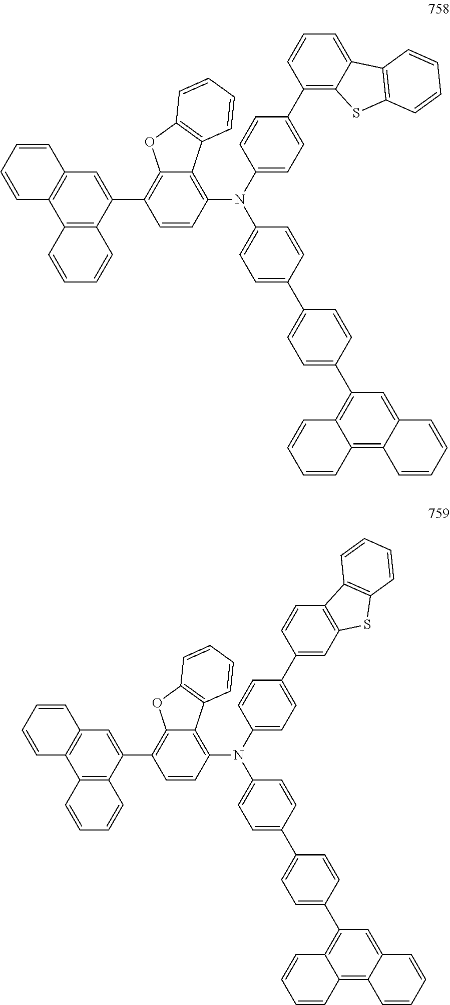

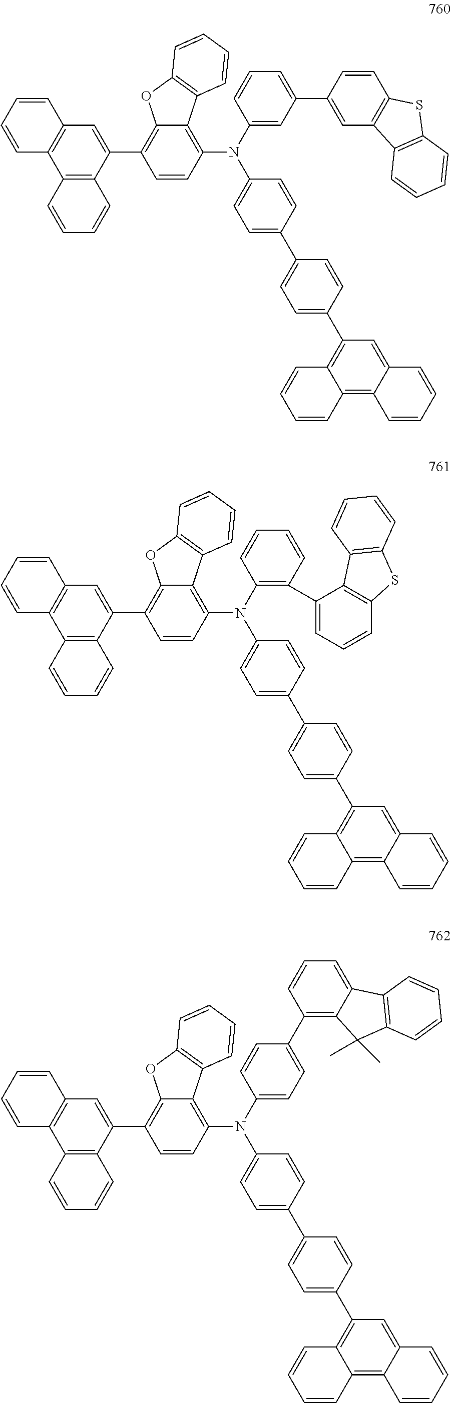

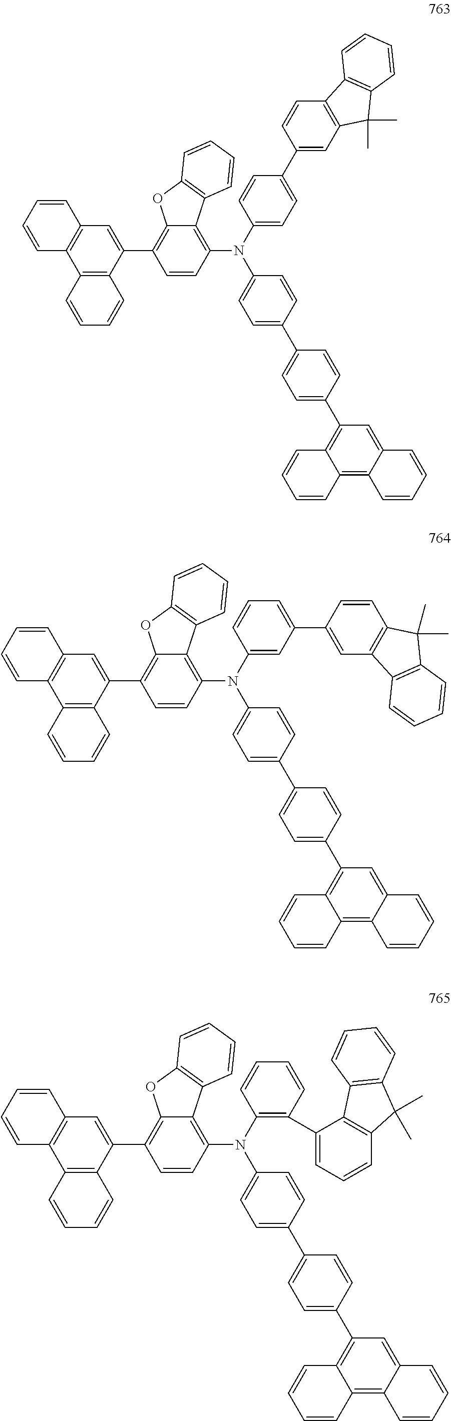

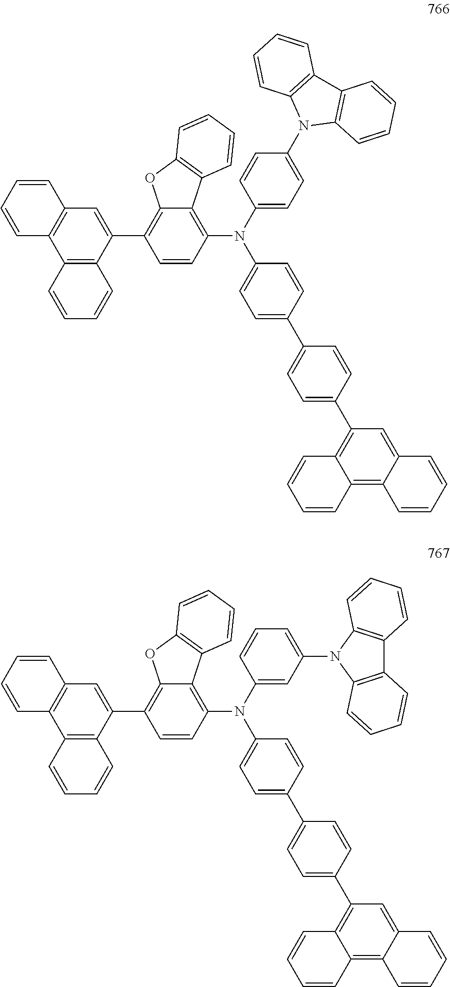

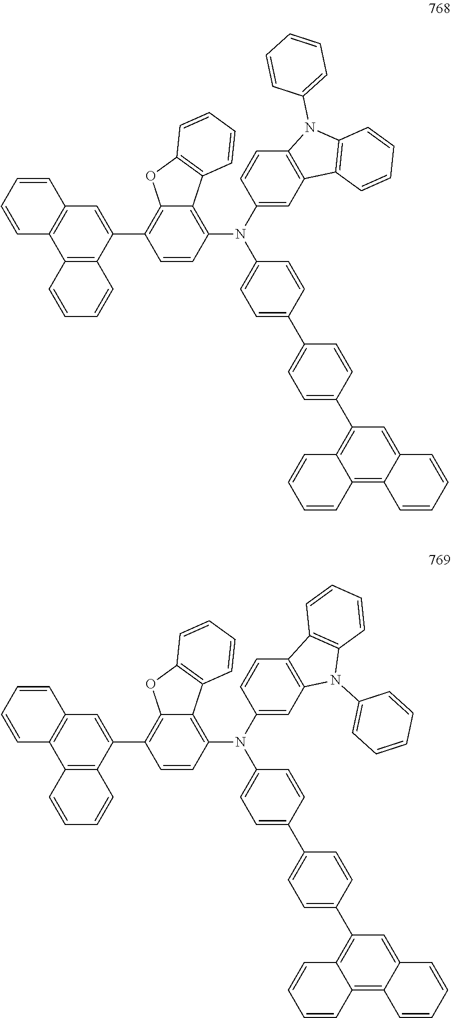 [SMILES: c1ccc(-n2c3ccccc3c3cc(N(c4ccc(-c5ccc(-c6cc7ccccc7c7ccccc67)cc5)cc4)c4ccc(-c5cc6ccccc6c6ccccc56)c5oc6ccccc6c45)ccc32)cc1 ; c1ccc(-n2c3ccccc3c3ccc(N(c4ccc(-c5ccc(-c6cc7ccccc7c7ccccc67)cc5)cc4)c4ccc(-c5cc6ccccc6c6ccccc56)c5oc6ccccc6c45)cc32)cc1]

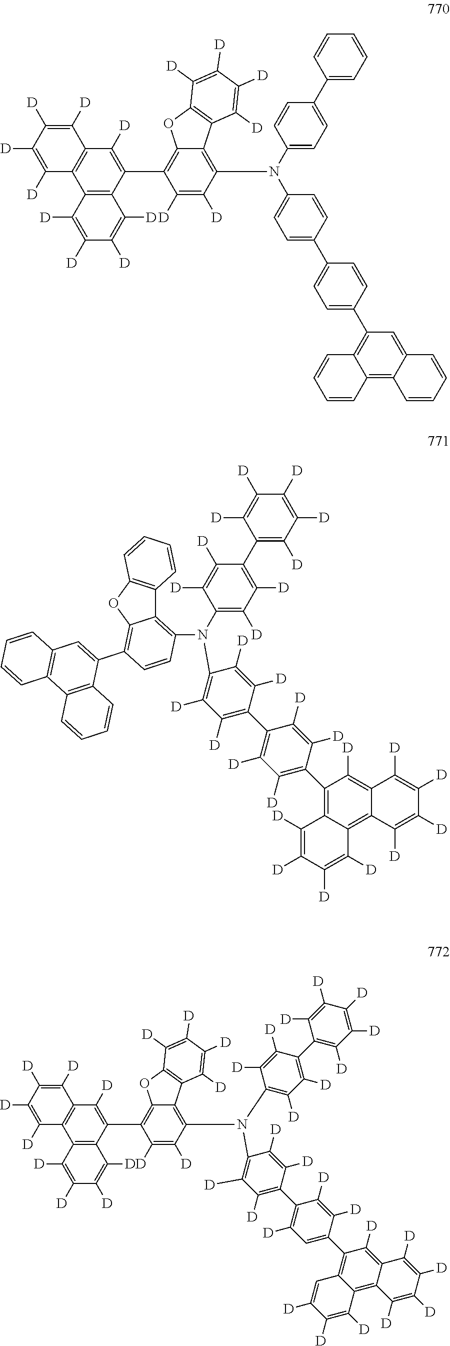

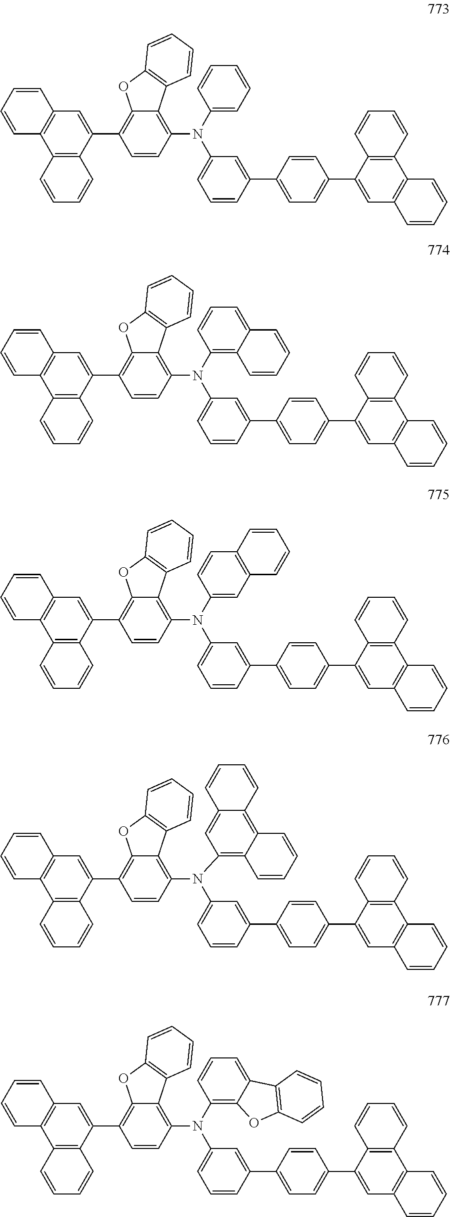

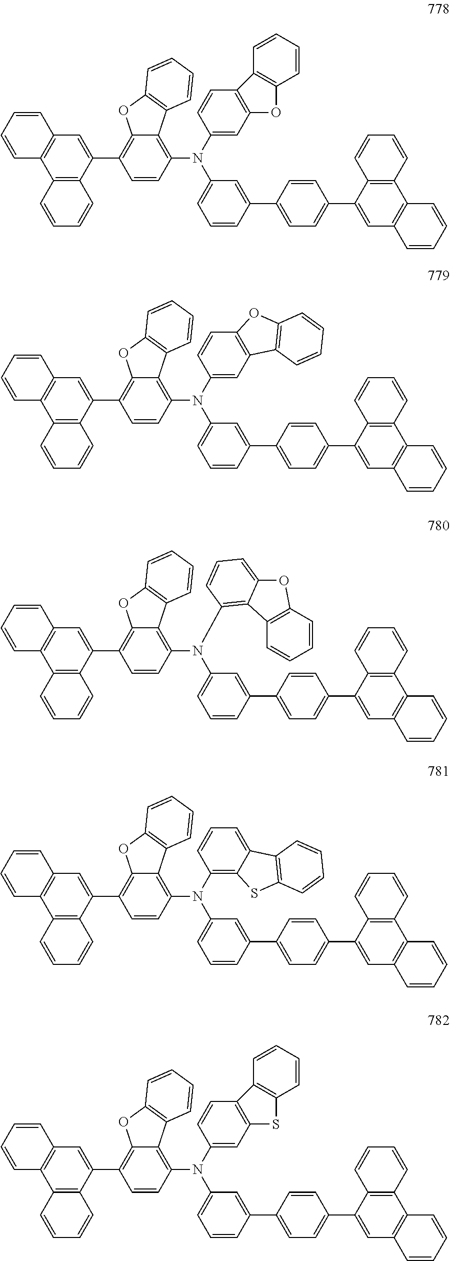

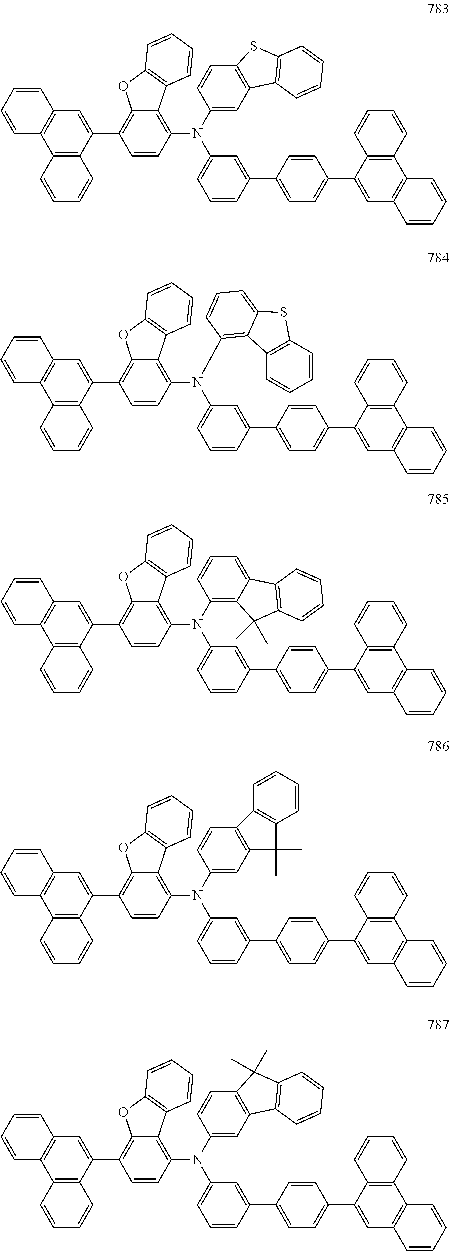

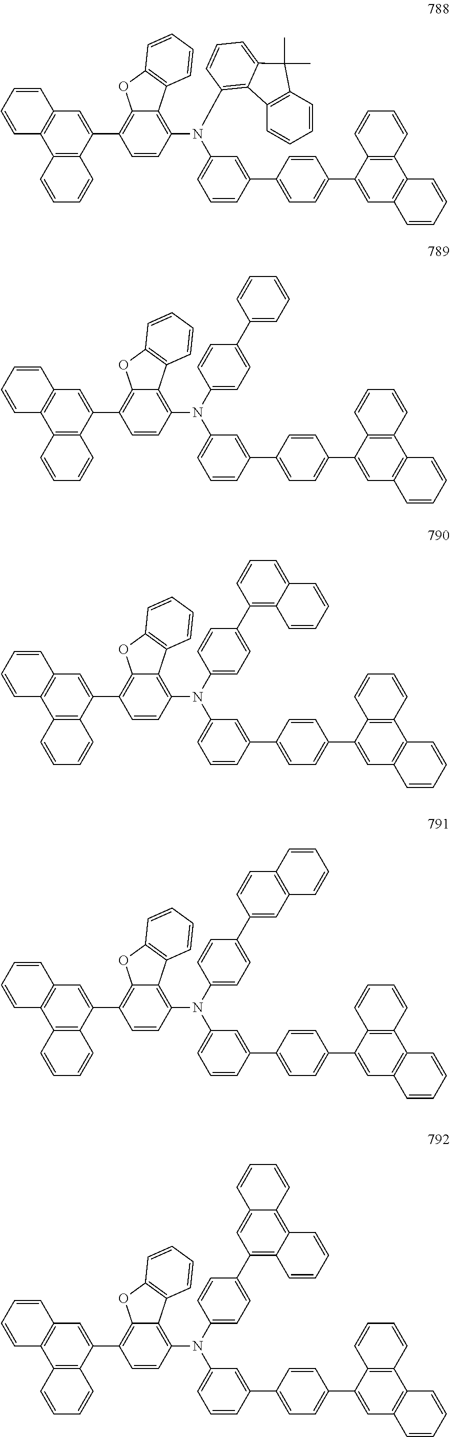

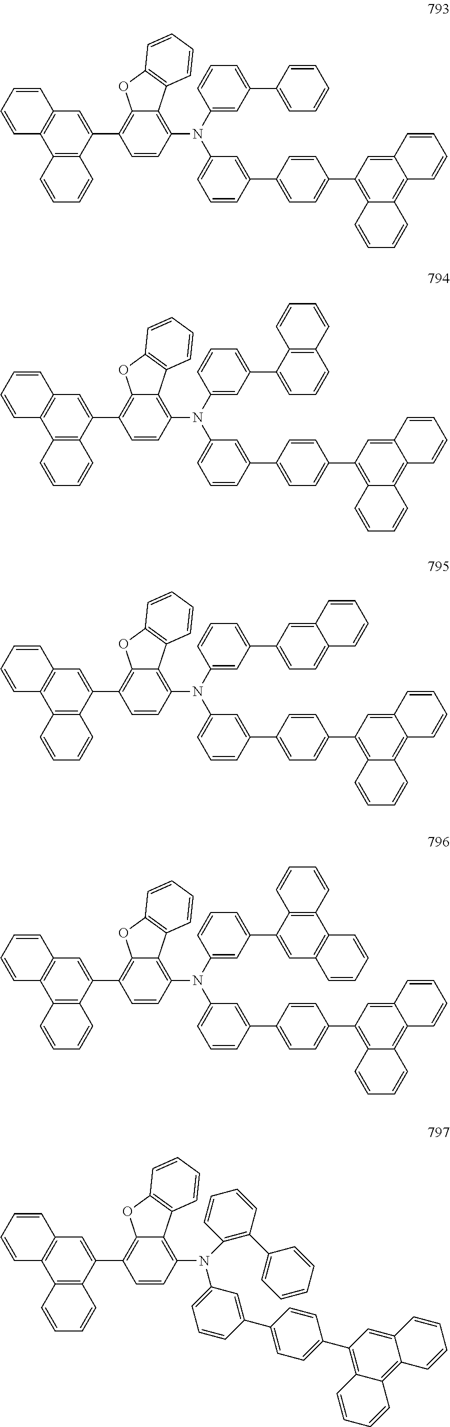

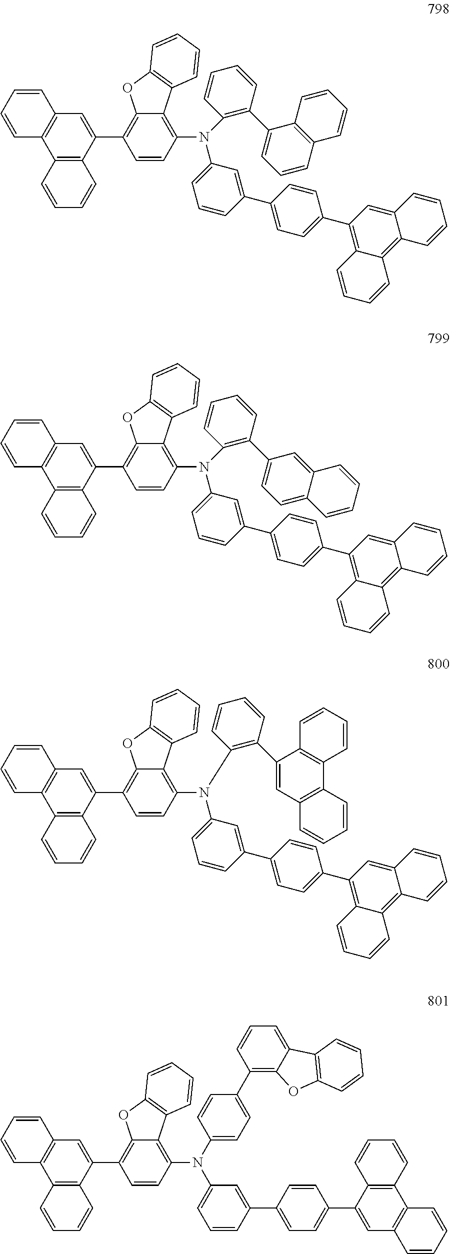

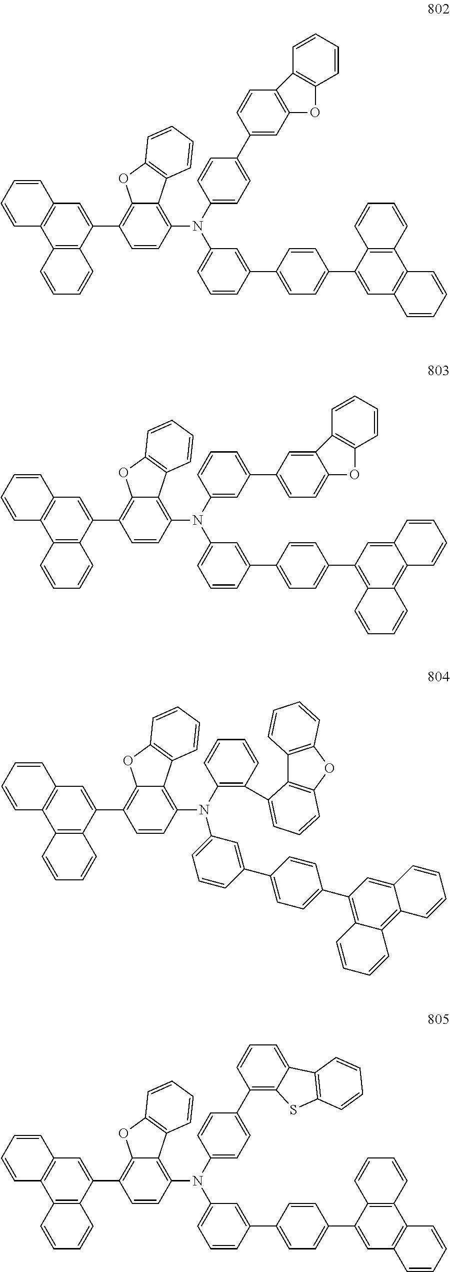

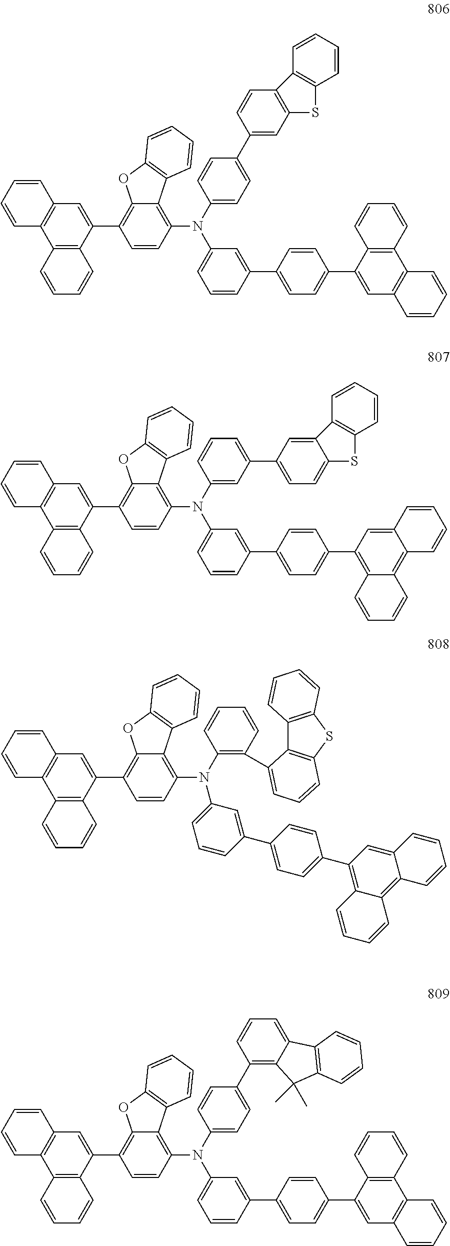 [SMILES: c1cc(-c2ccc(-c3cc4ccccc4c4ccccc34)cc2)cc(N(c2ccc(-c3ccc4c(c3)sc3ccccc34)cc2)c2ccc(-c3cc4ccccc4c4ccccc34)c3oc4ccccc4c23)c1 ; c1cc(-c2ccc(-c3cc4ccccc4c4ccccc34)cc2)cc(N(c2cccc(-c3ccc4sc5ccccc5c4c3)c2)c2ccc(-c3cc4ccccc4c4ccccc34)c3oc4ccccc4c23)c1 ; c1cc(-c2ccc(-c3cc4ccccc4c4ccccc34)cc2)cc(N(c2ccccc2-c2cccc3sc4ccccc4c23)c2ccc(-c3cc4ccccc4c4ccccc34)c3oc4ccccc4c23)c1 ; CC1(C)c2ccccc2-c2cccc(-c3ccc(N(c4cccc(-c5ccc(-c6cc7ccccc7c7ccccc67)cc5)c4)c4ccc(-c5cc6ccccc6c6ccccc56)c5oc6ccccc6c45)cc3)c21]

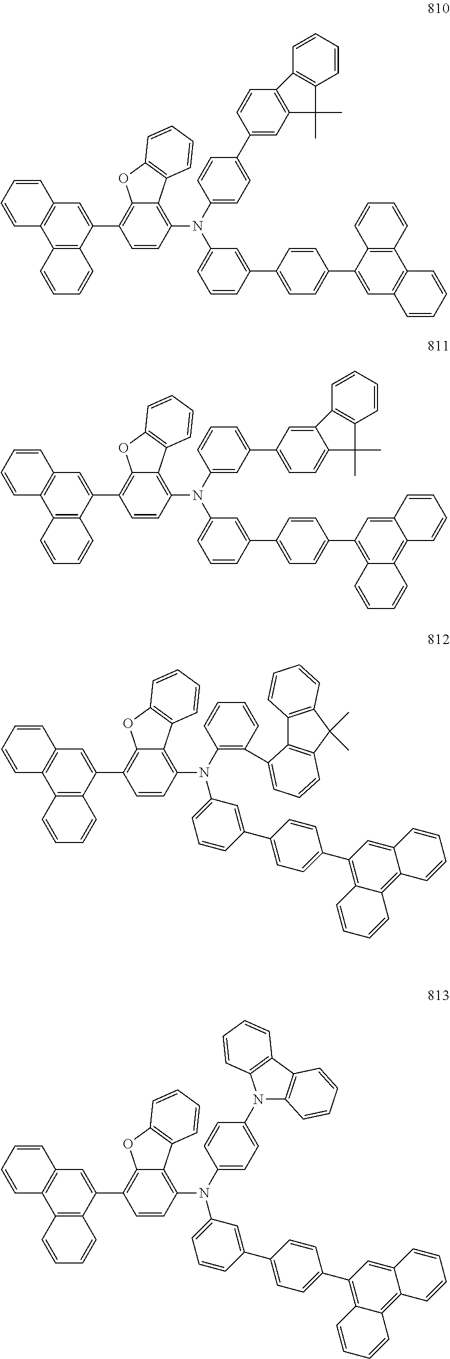 [SMILES: CC1(C)c2ccccc2-c2ccc(-c3ccc(N(c4cccc(-c5ccc(-c6cc7ccccc7c7ccccc67)cc5)c4)c4ccc(-c5cc6ccccc6c6ccccc56)c5oc6ccccc6c45)cc3)cc21 ; CC1(C)c2ccccc2-c2cc(-c3cccc(N(c4cccc(-c5ccc(-c6cc7ccccc7c7ccccc67)cc5)c4)c4ccc(-c5cc6ccccc6c6ccccc56)c5oc6ccccc6c45)c3)ccc21 ; CC1(C)c2ccccc2-c2c(-c3ccccc3N(c3cccc(-c4ccc(-c5cc6ccccc6c6ccccc56)cc4)c3)c3ccc(-c4cc5ccccc5c5ccccc45)c4oc5ccccc5c34)cccc21 ; c1cc(-c2ccc(-c3cc4ccccc4c4ccccc34)cc2)cc(N(c2ccc(-n3c4ccccc4c4ccccc43)cc2)c2ccc(-c3cc4ccccc4c4ccccc34)c3oc4ccccc4c23)c1]

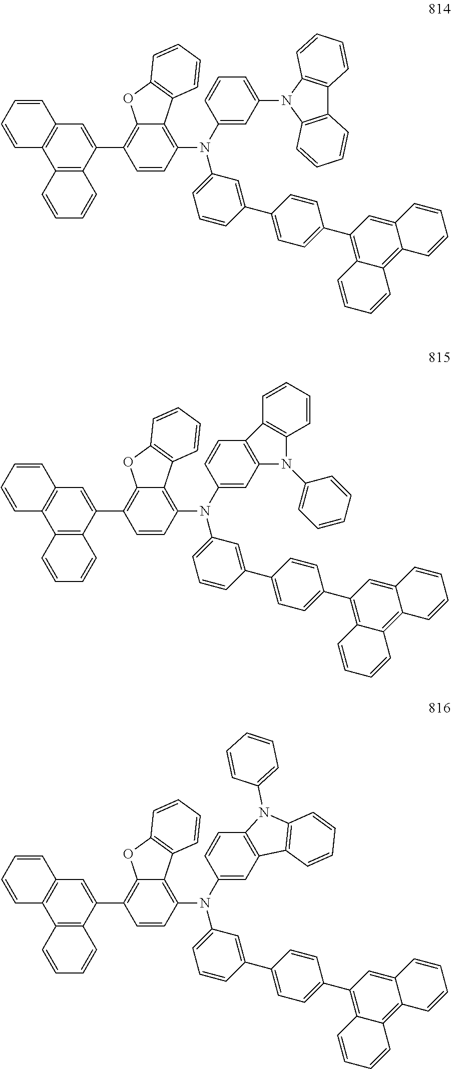 [SMILES: c1cc(-c2ccc(-c3cc4ccccc4c4ccccc34)cc2)cc(N(c2cccc(-n3c4ccccc4c4ccccc43)c2)c2ccc(-c3cc4ccccc4c4ccccc34)c3oc4ccccc4c23)c1 ; c1ccc(-n2c3ccccc3c3ccc(N(c4cccc(-c5ccc(-c6cc7ccccc7c7ccccc67)cc5)c4)c4ccc(-c5cc6ccccc6c6ccccc56)c5oc6ccccc6c45)cc32)cc1 ; c1ccc(-n2c3ccccc3c3cc(N(c4cccc(-c5ccc(-c6cc7ccccc7c7ccccc67)cc5)c4)c4ccc(-c5cc6ccccc6c6ccccc56)c5oc6ccccc6c45)ccc32)cc1]

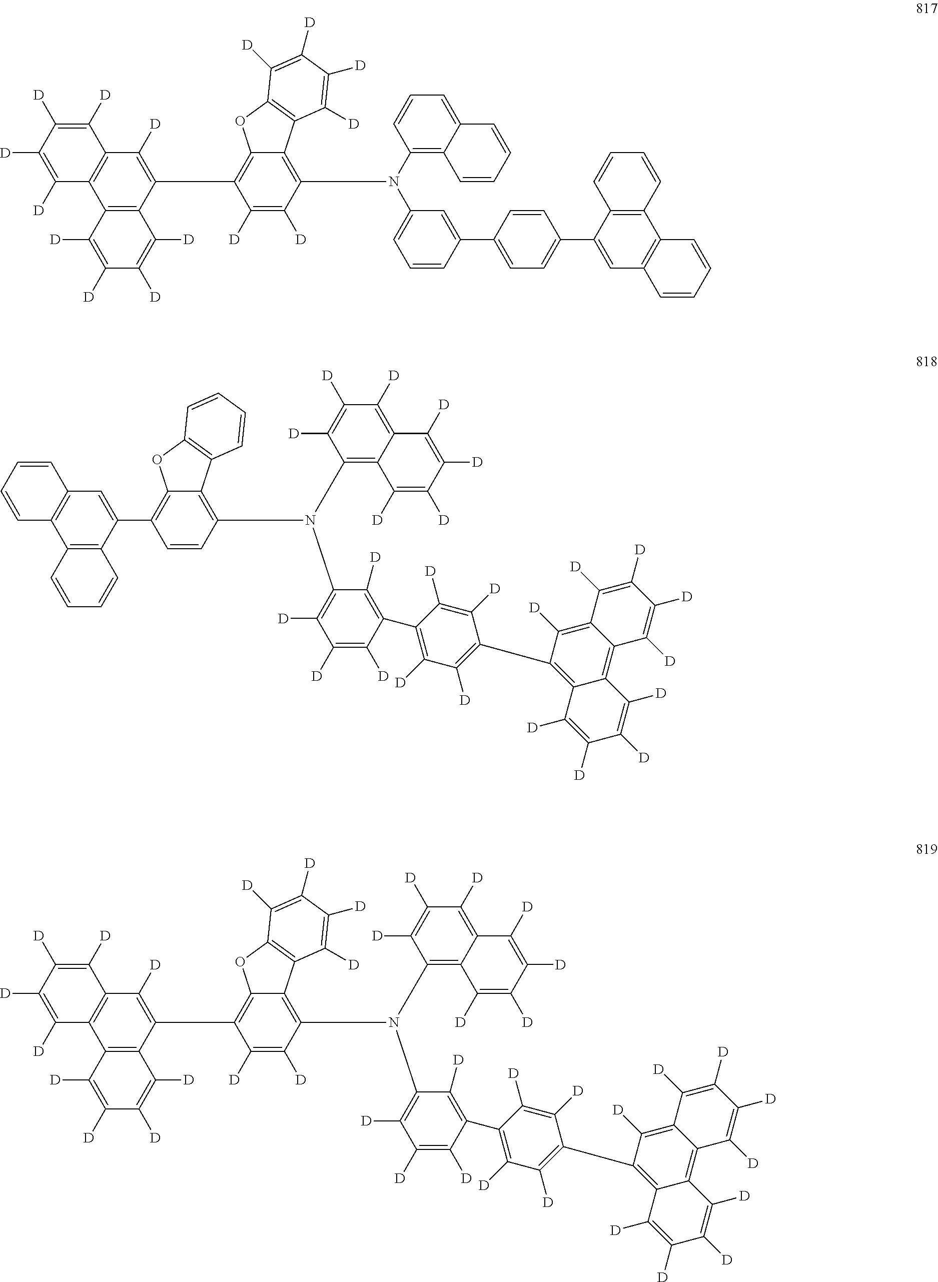

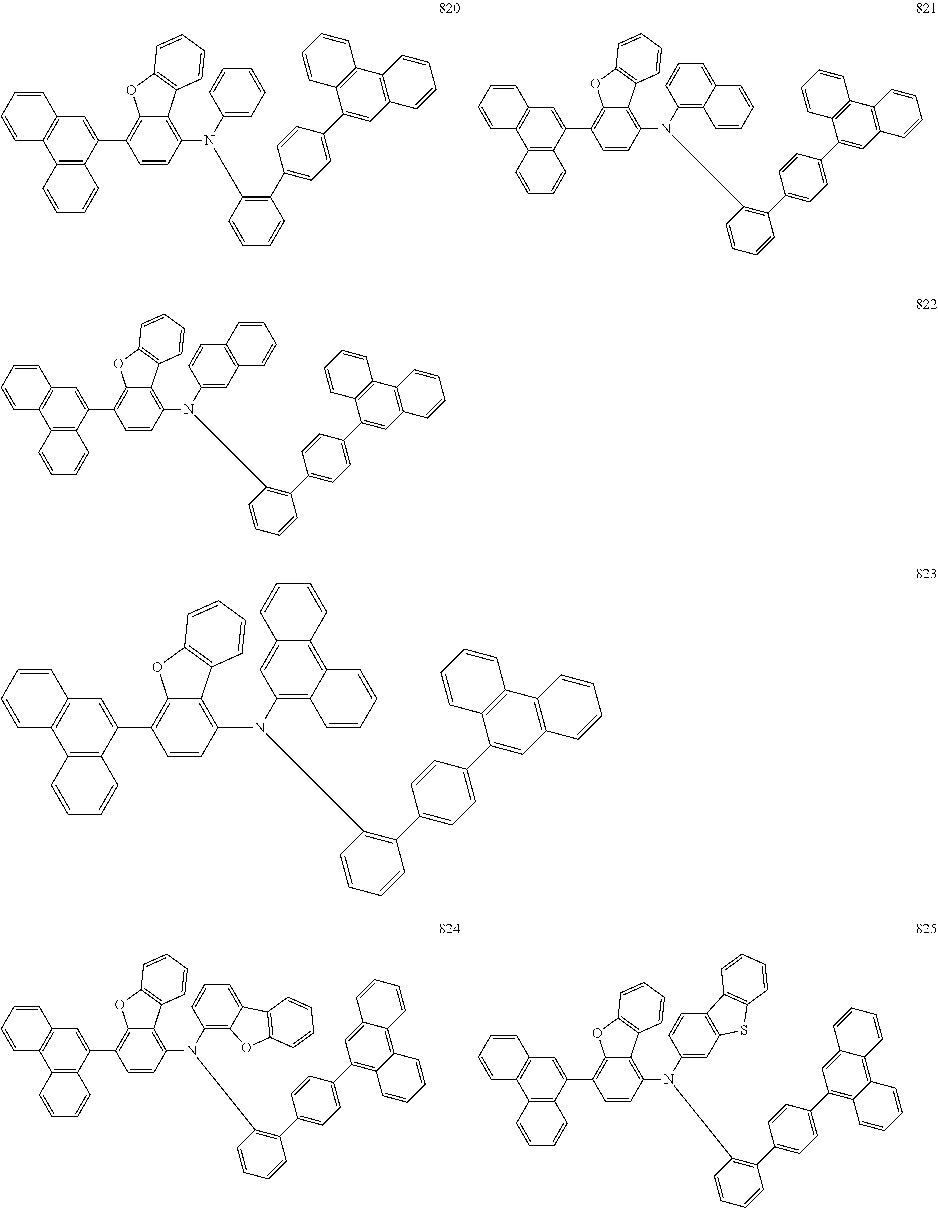

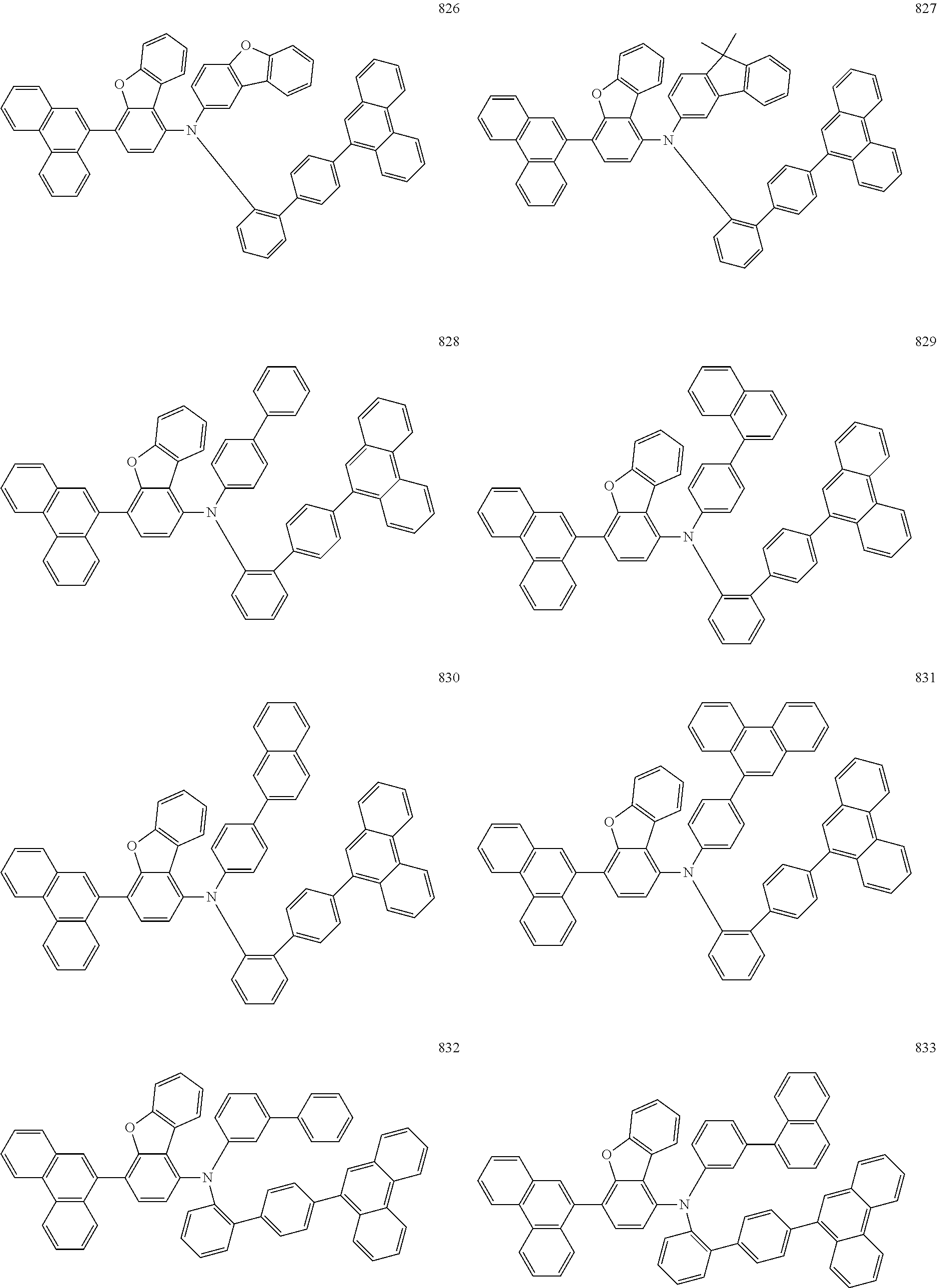

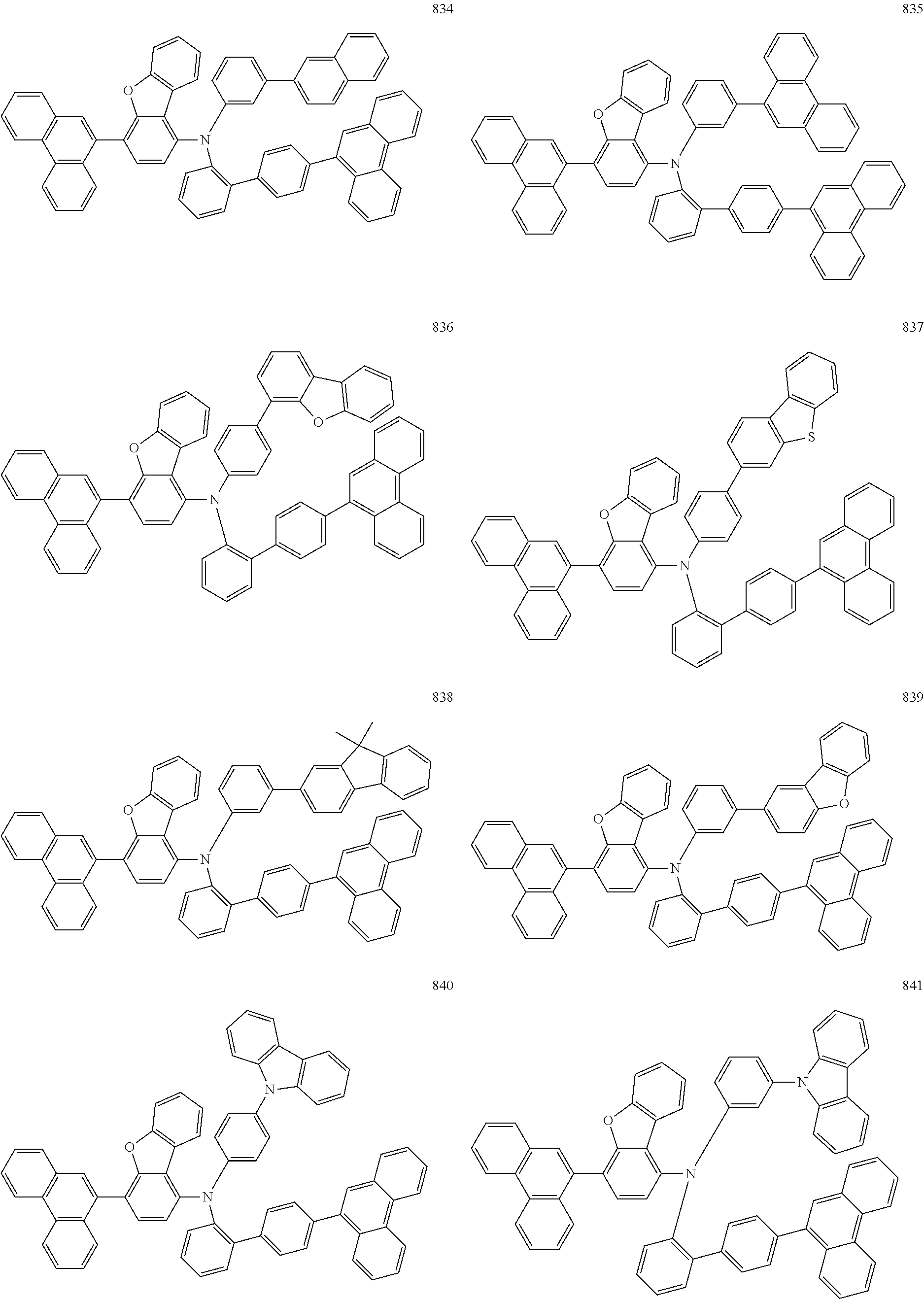

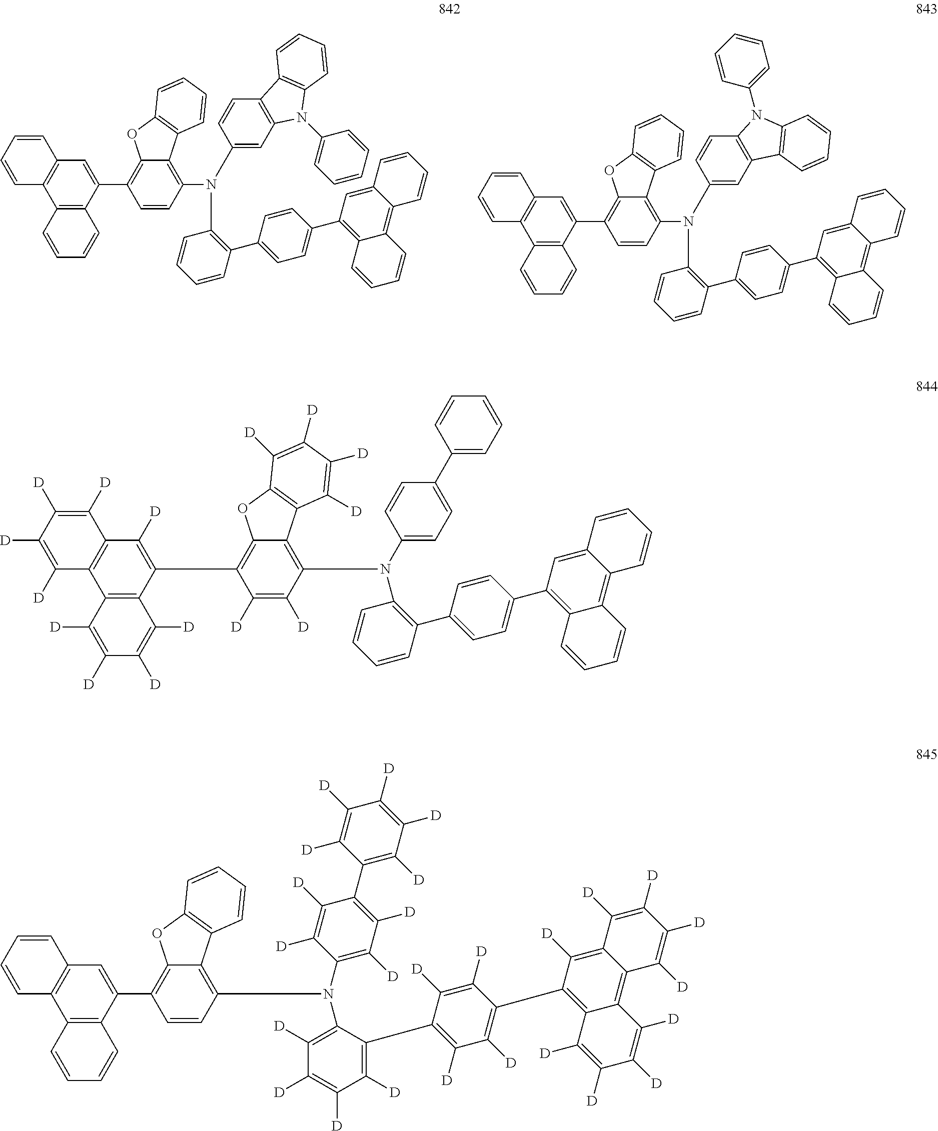

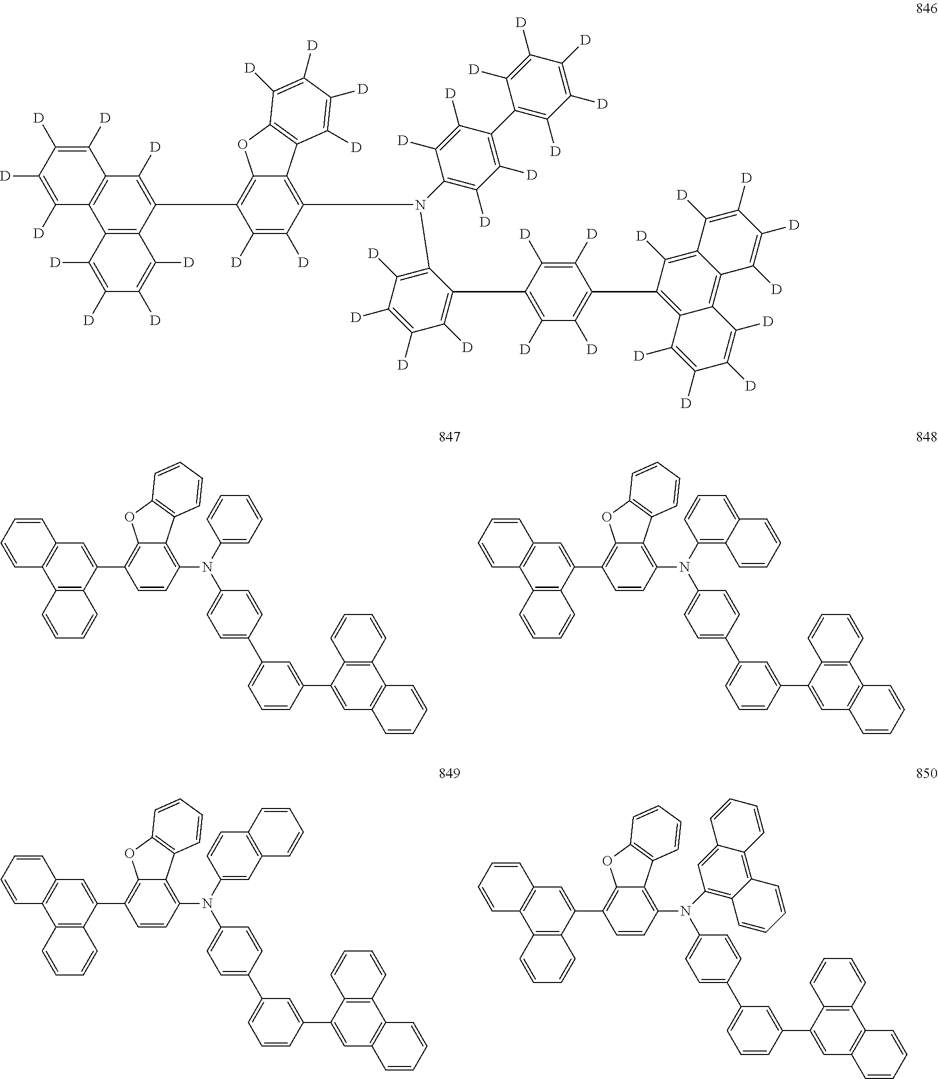

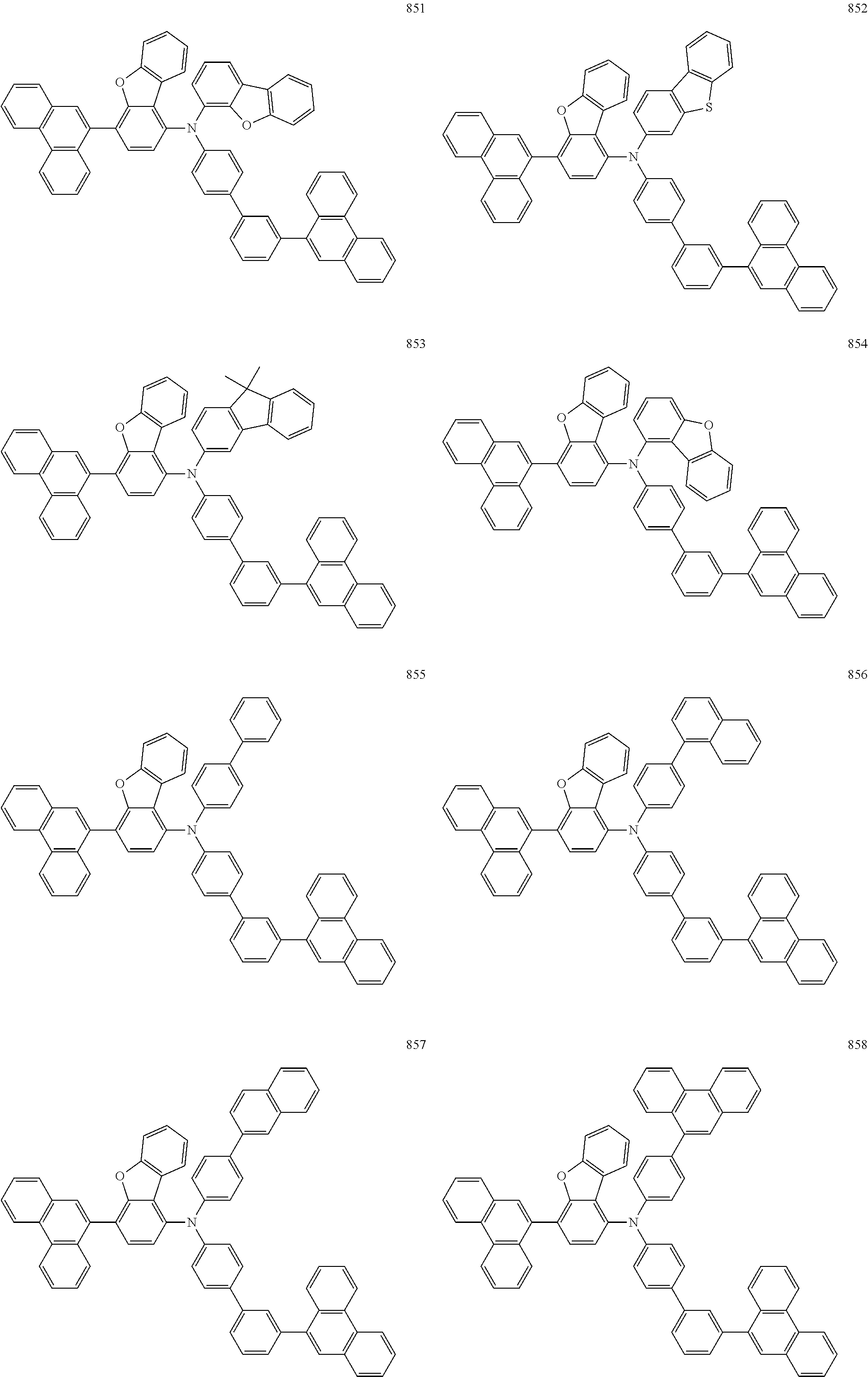

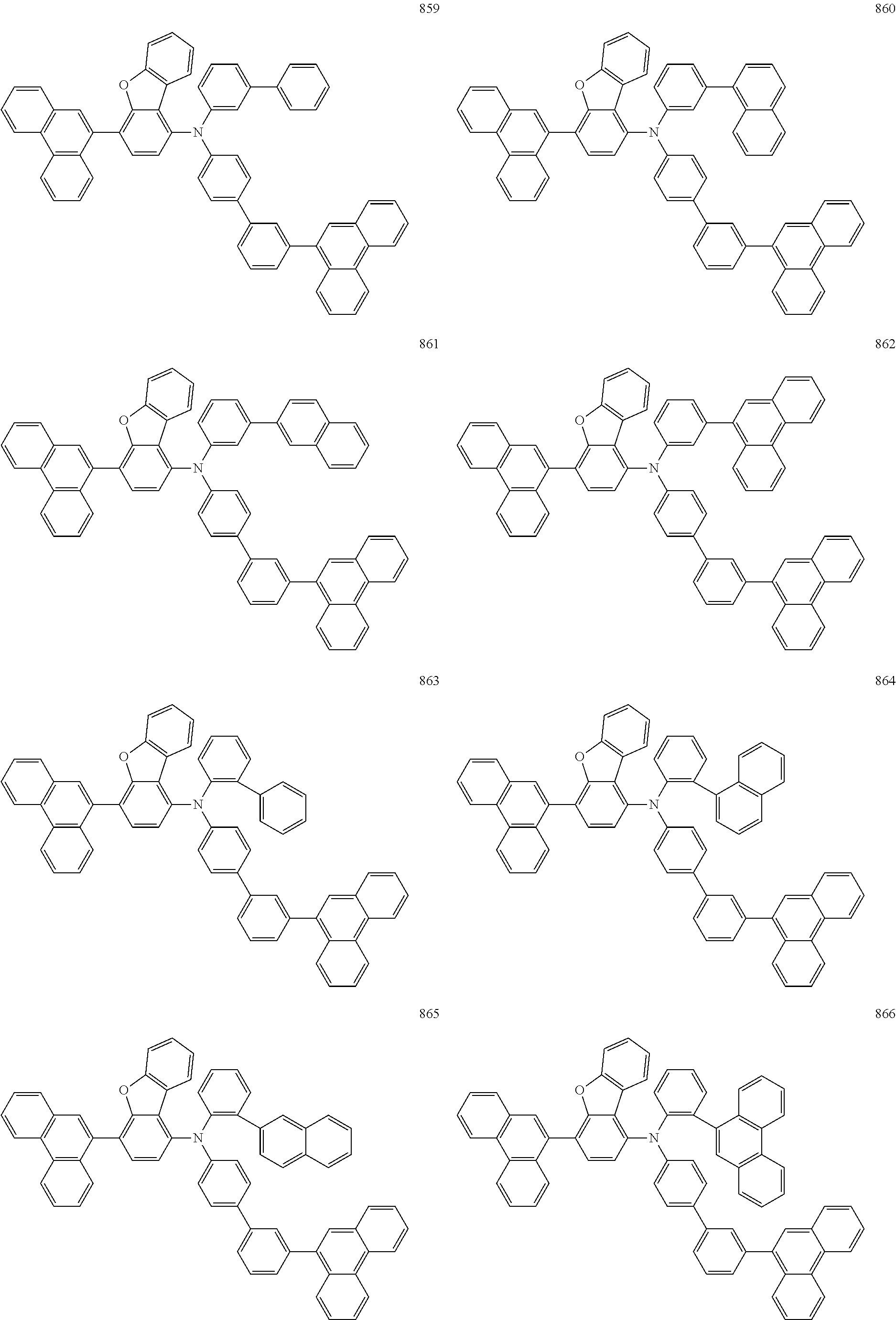

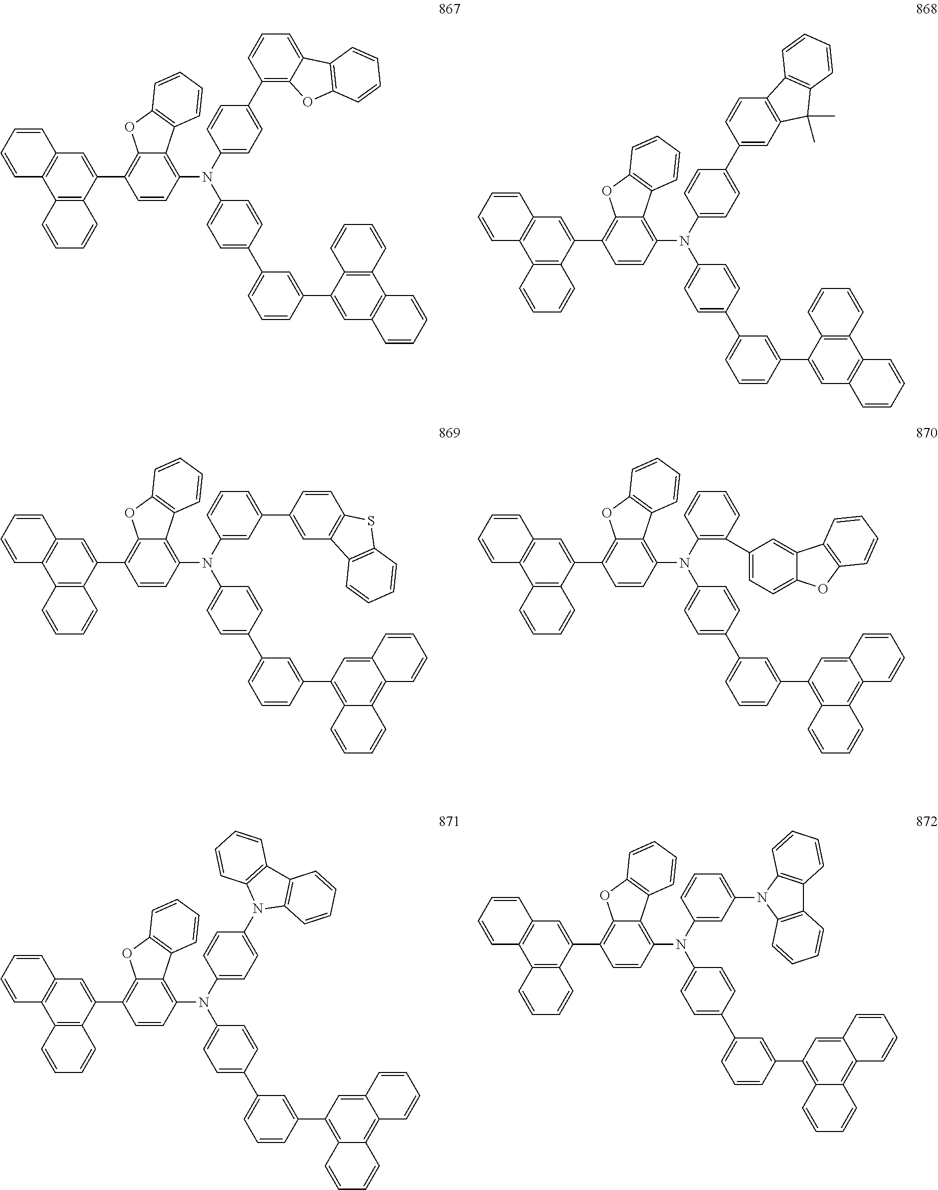

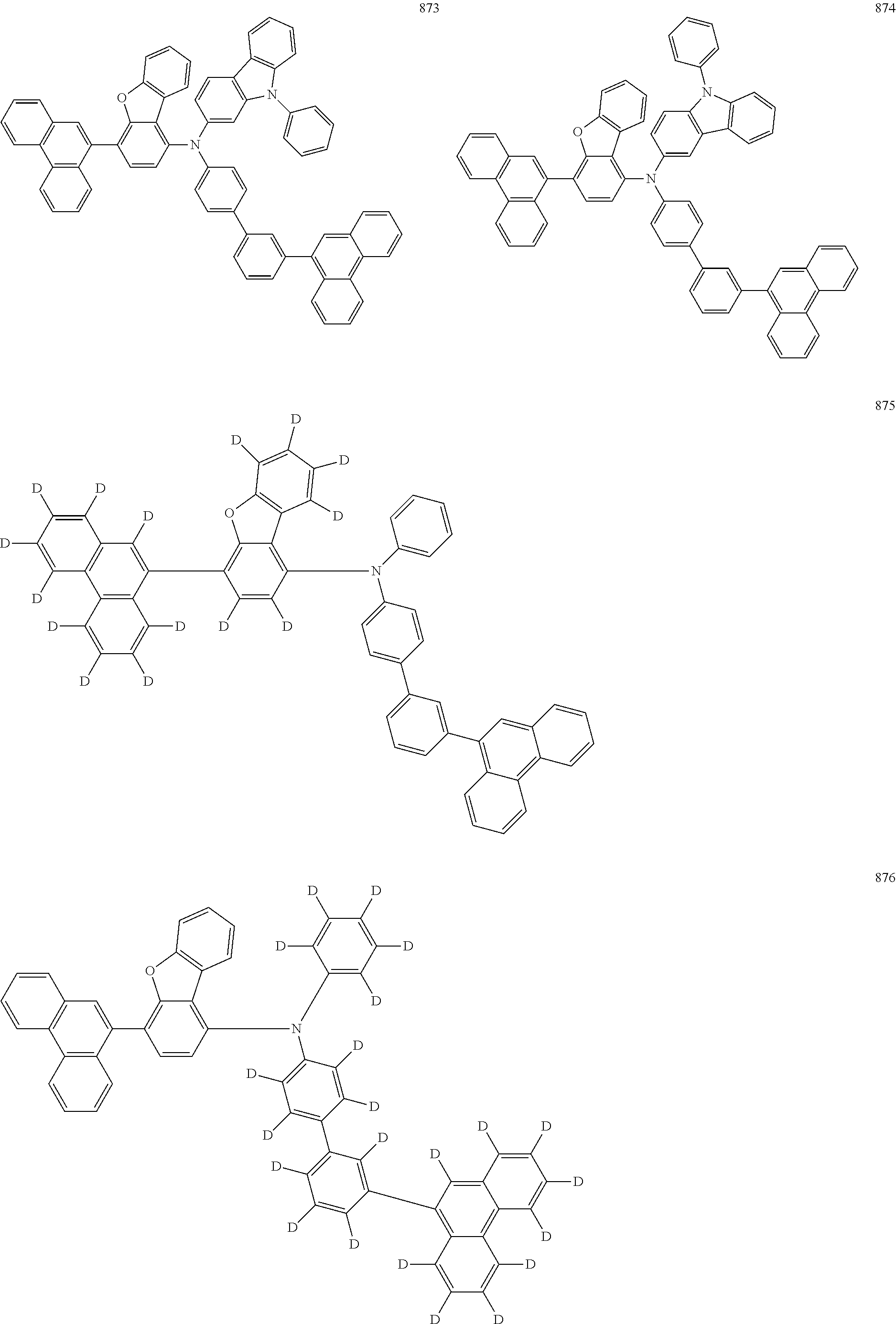

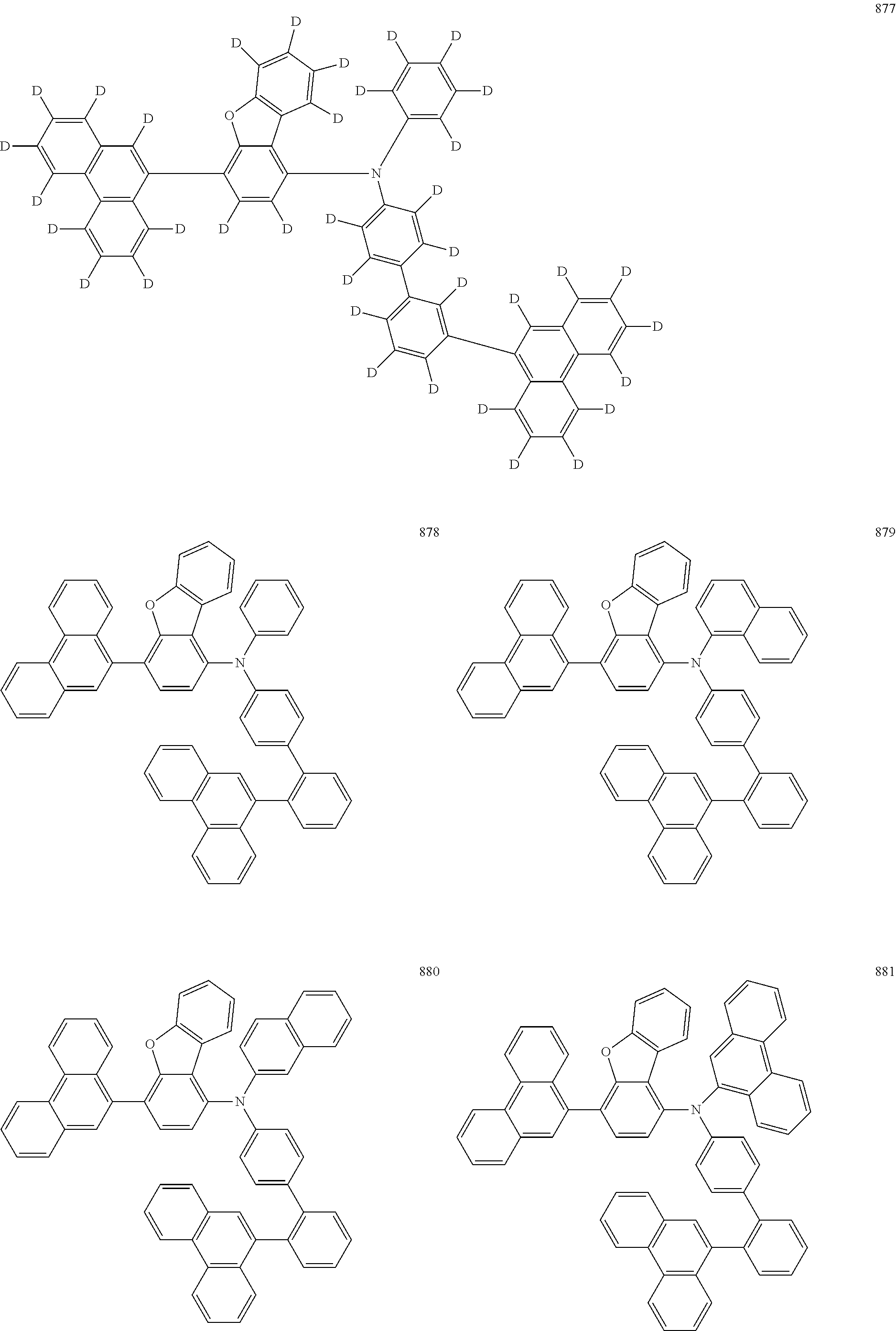

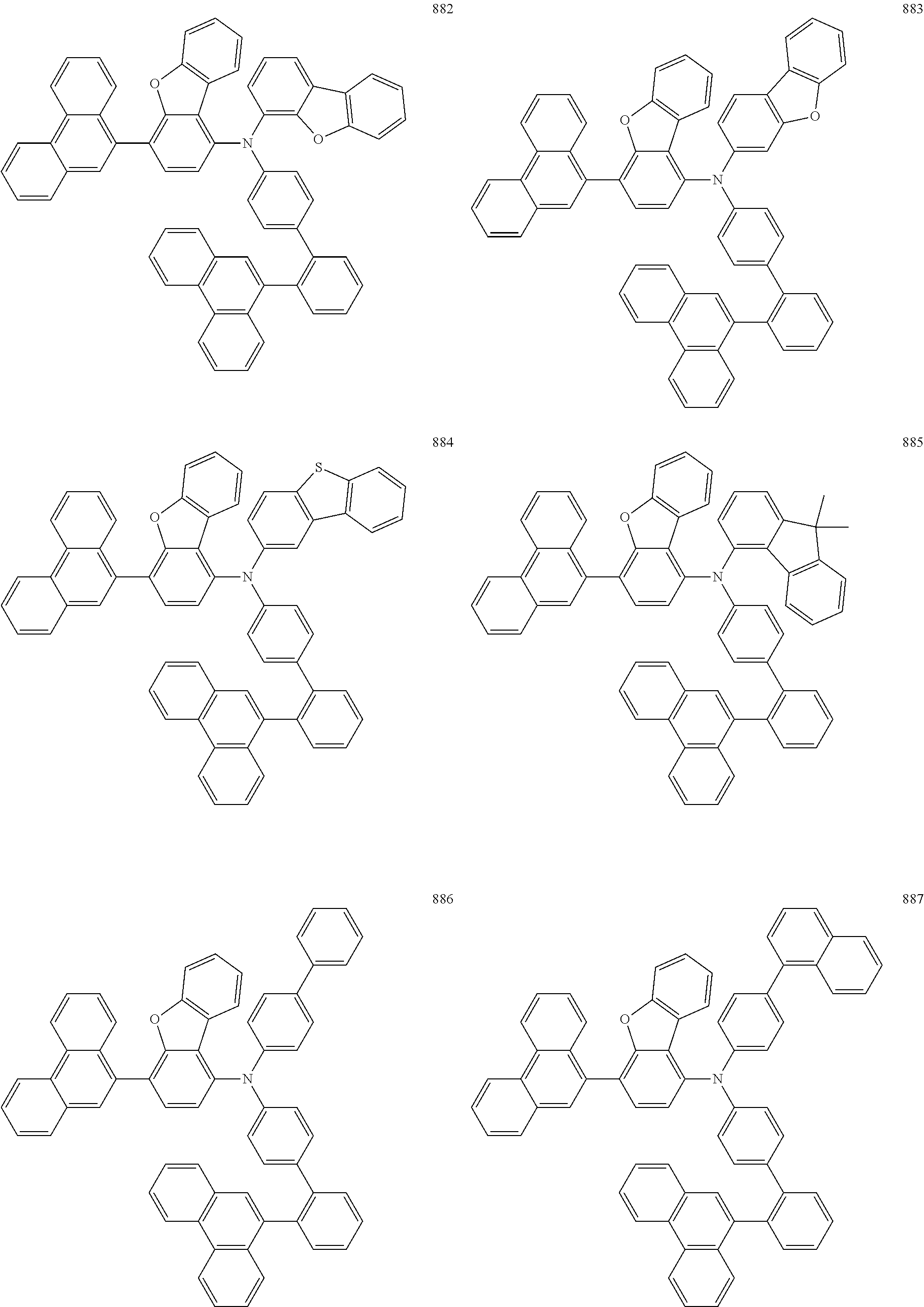

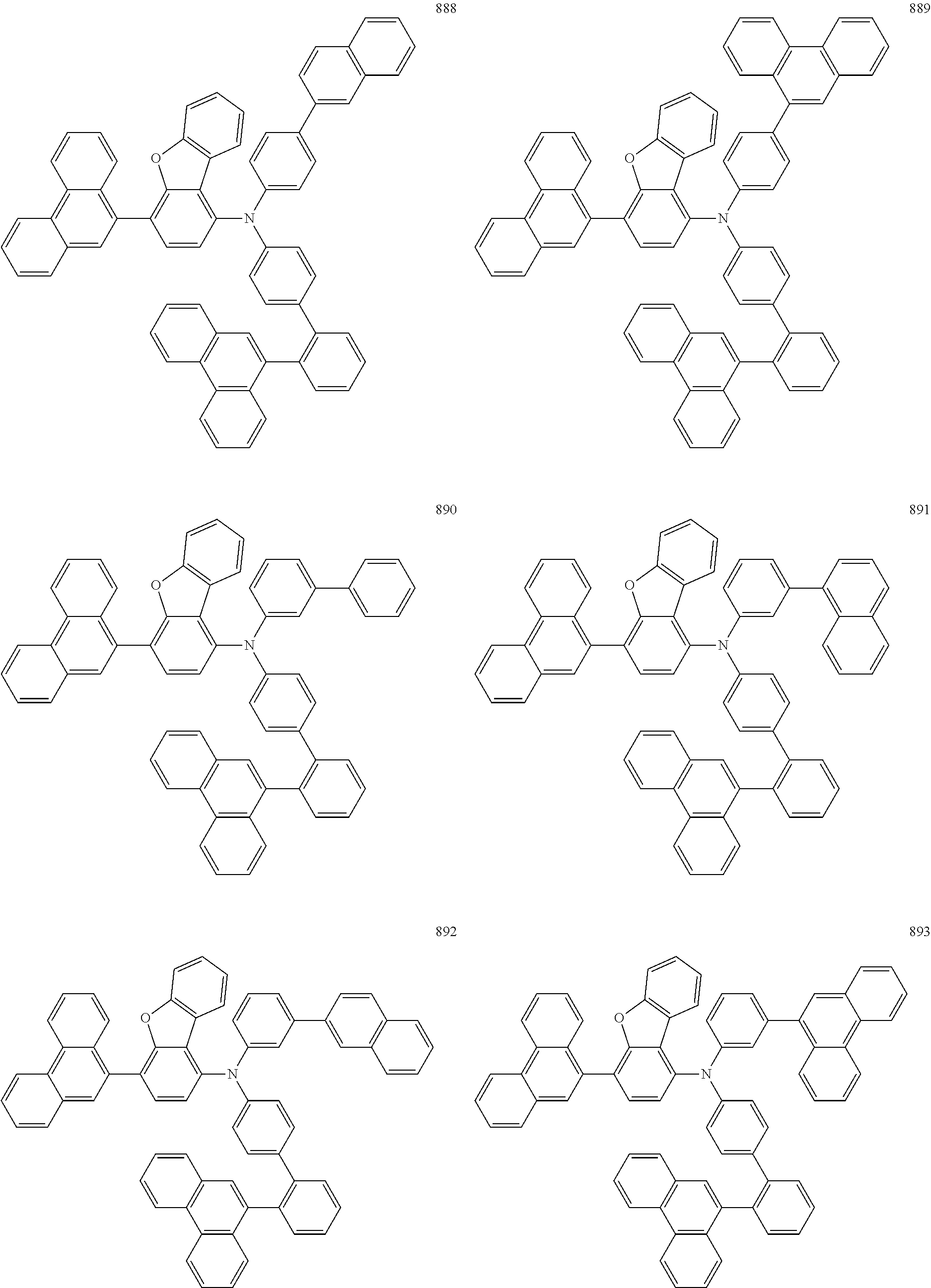

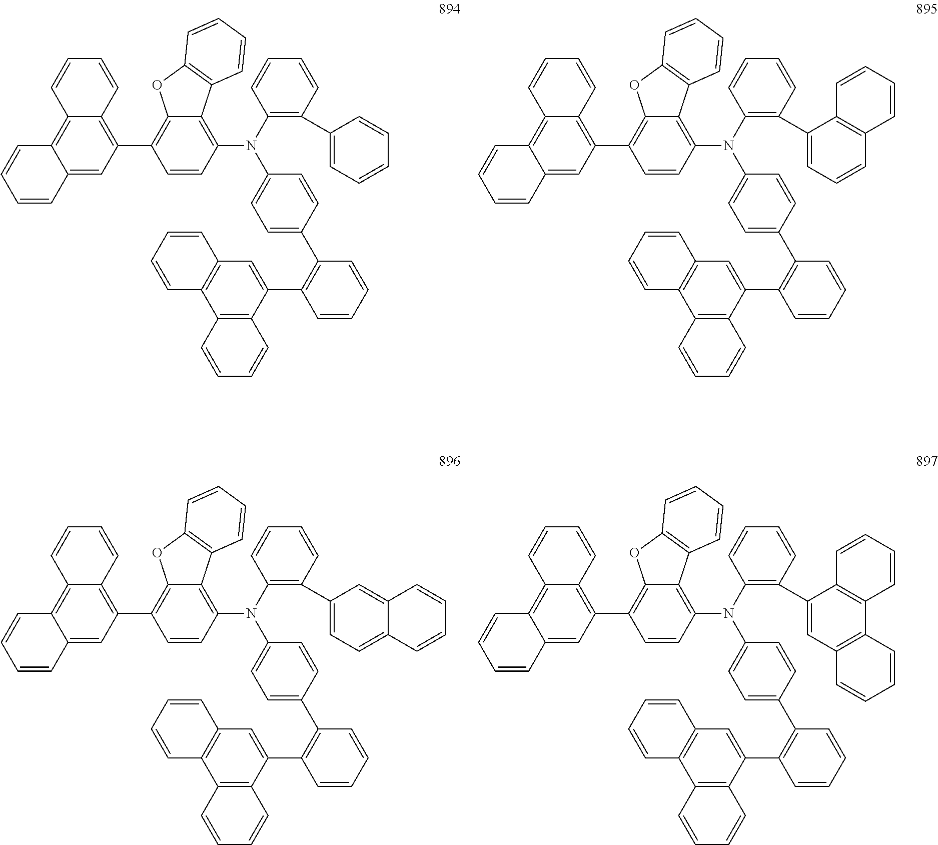

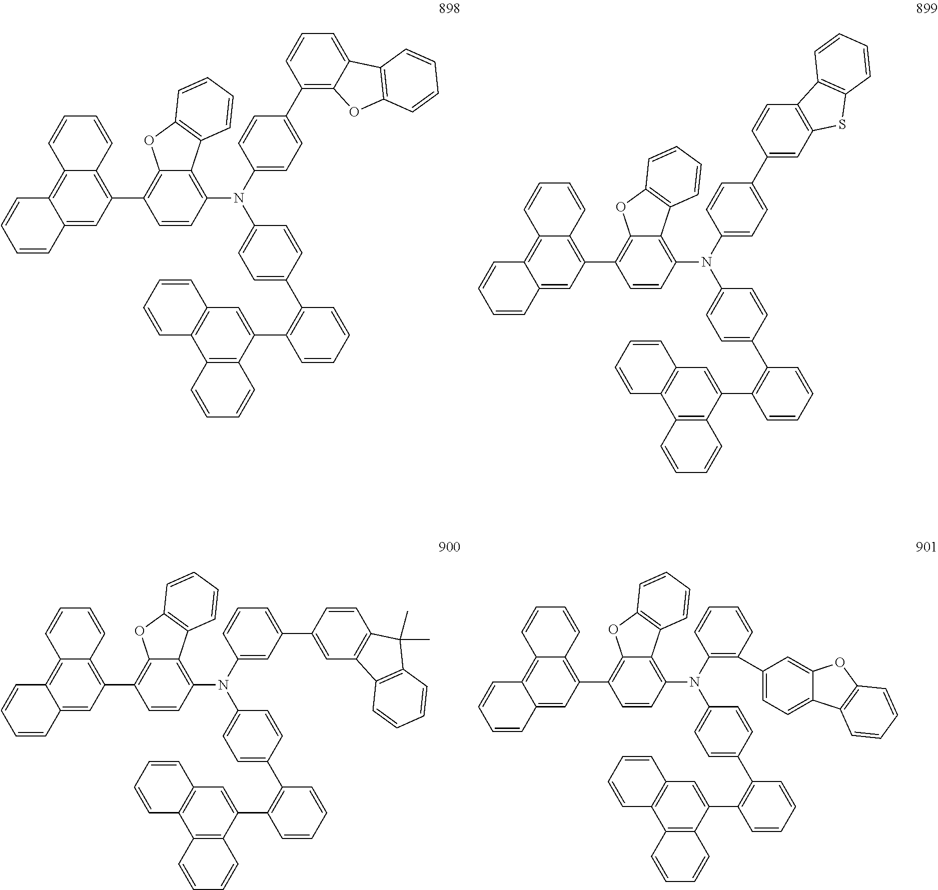 [SMILES: c1ccc(-c2cc3ccccc3c3ccccc23)c(-c2ccc(N(c3ccc(-c4cccc5c4oc4ccccc45)cc3)c3ccc(-c4cc5ccccc5c5ccccc45)c4oc5ccccc5c34)cc2)c1 ; c1ccc(-c2cc3ccccc3c3ccccc23)c(-c2ccc(N(c3ccc(-c4ccc5c(c4)sc4ccccc45)cc3)c3ccc(-c4cc5ccccc5c5ccccc45)c4oc5ccccc5c34)cc2)c1 ; CC1(C)c2ccccc2-c2cc(-c3cccc(N(c4ccc(-c5ccccc5-c5cc6ccccc6c6ccccc56)cc4)c4ccc(-c5cc6ccccc6c6ccccc56)c5oc6ccccc6c45)c3)ccc21 ; c1ccc(-c2cc3ccccc3c3ccccc23)c(-c2ccc(N(c3ccccc3-c3ccc4c(c3)oc3ccccc34)c3ccc(-c4cc5ccccc5c5ccccc45)c4oc5ccccc5c34)cc2)c1]

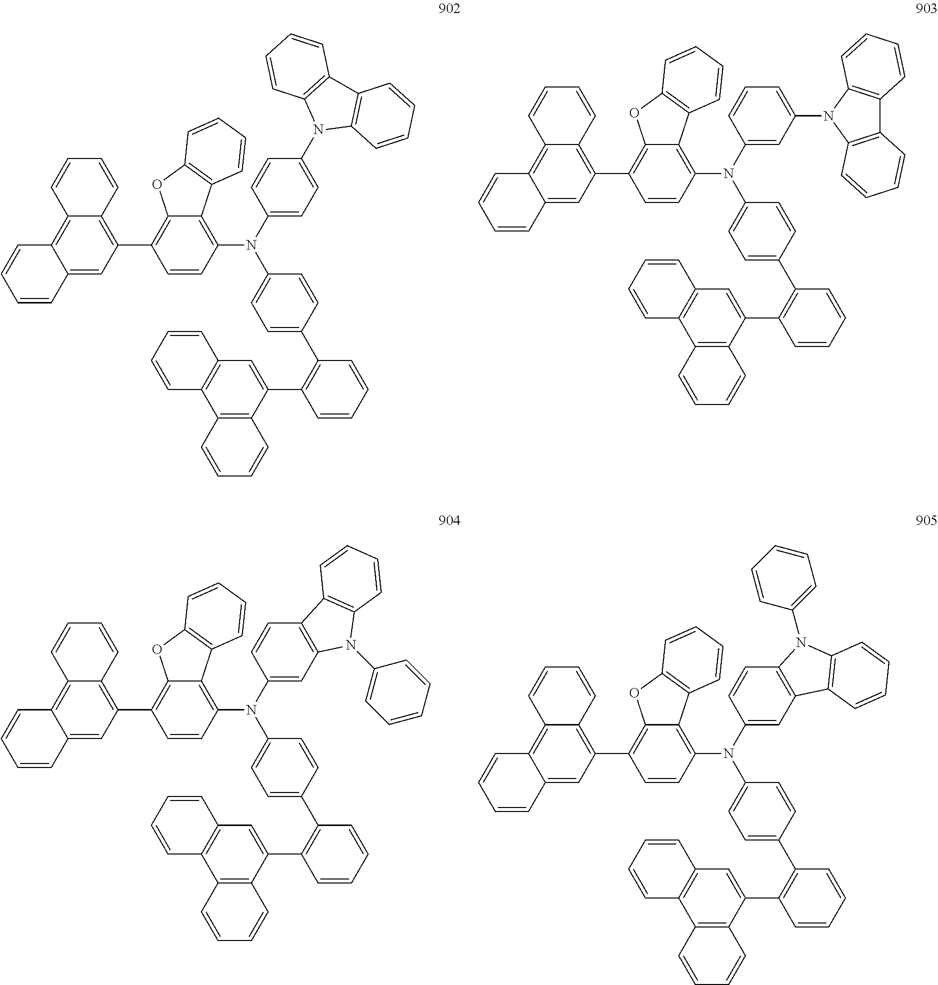 [SMILES: c1ccc(-c2cc3ccccc3c3ccccc23)c(-c2ccc(N(c3ccc(-n4c5ccccc5c5ccccc54)cc3)c3ccc(-c4cc5ccccc5c5ccccc45)c4oc5ccccc5c34)cc2)c1 ; c1cc(N(c2ccc(-c3ccccc3-c3cc4ccccc4c4ccccc34)cc2)c2ccc(-c3cc4ccccc4c4ccccc34)c3oc4ccccc4c23)cc(-n2c3ccccc3c3ccccc32)c1 ; c1ccc(-n2c3ccccc3c3ccc(N(c4ccc(-c5ccccc5-c5cc6ccccc6c6ccccc56)cc4)c4ccc(-c5cc6ccccc6c6ccccc56)c5oc6ccccc6c45)cc32)cc1 ; c1ccc(-n2c3ccccc3c3cc(N(c4ccc(-c5ccccc5-c5cc6ccccc6c6ccccc56)cc4)c4ccc(-c5cc6ccccc6c6ccccc56)c5oc6ccccc6c45)ccc32)cc1]

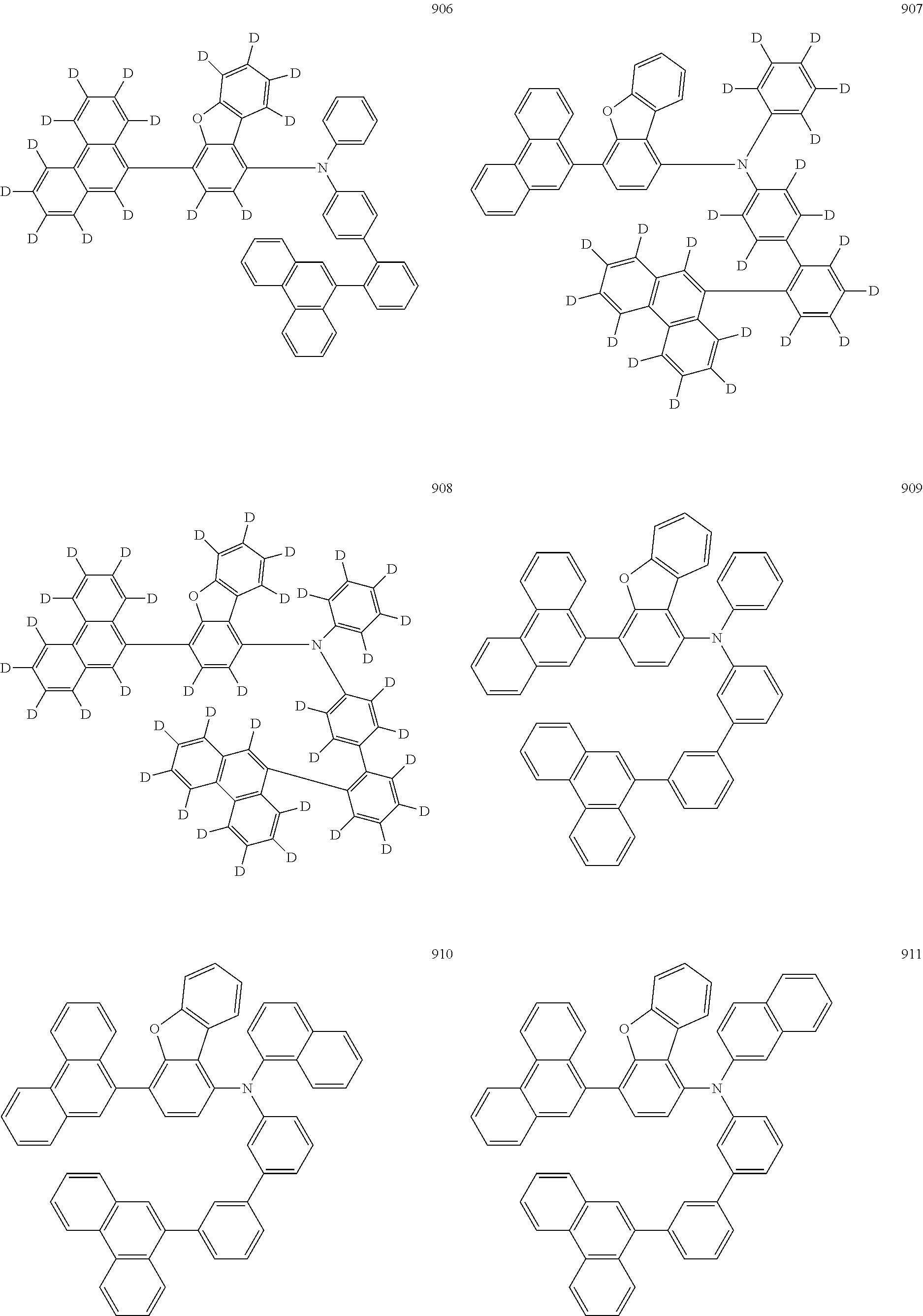

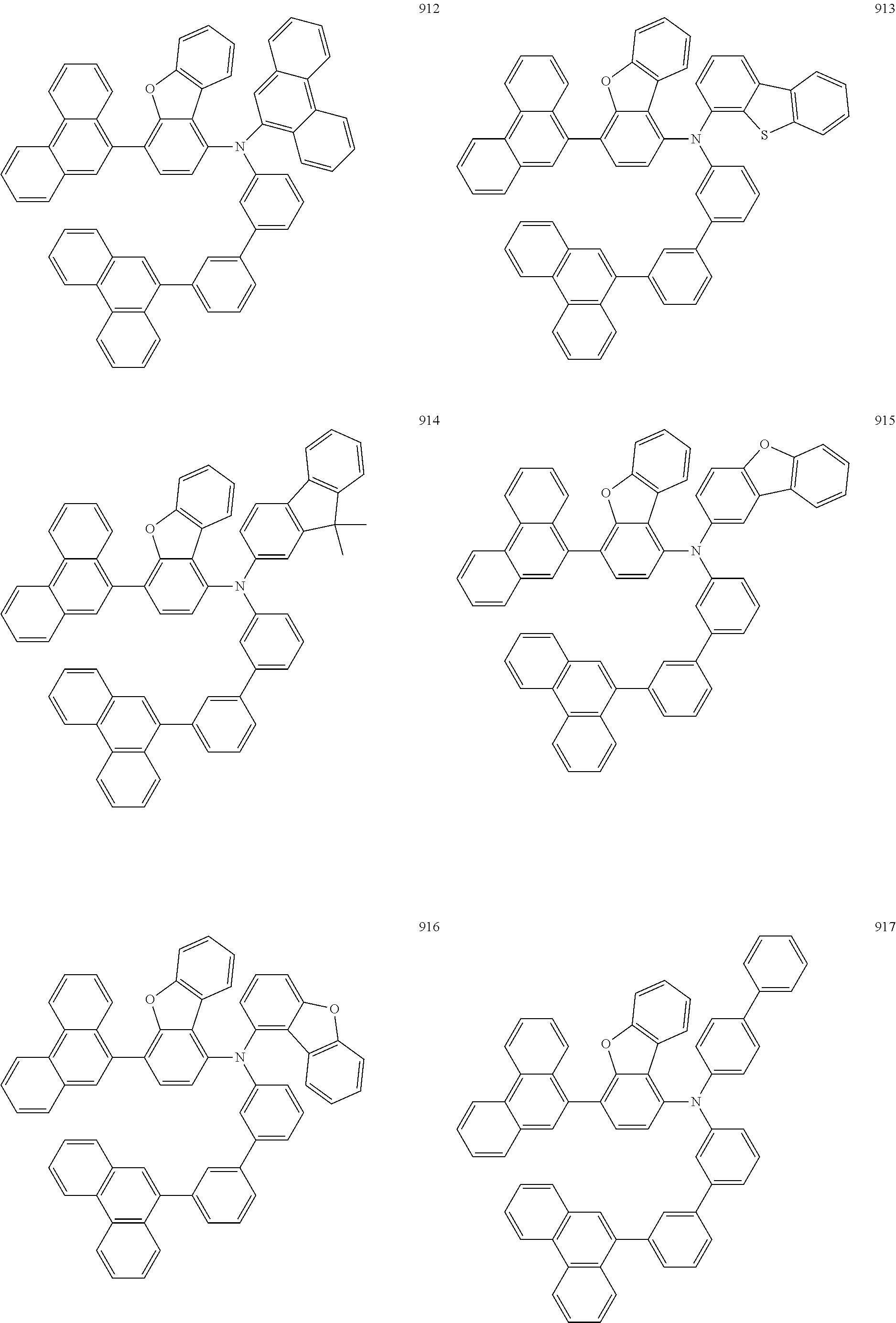

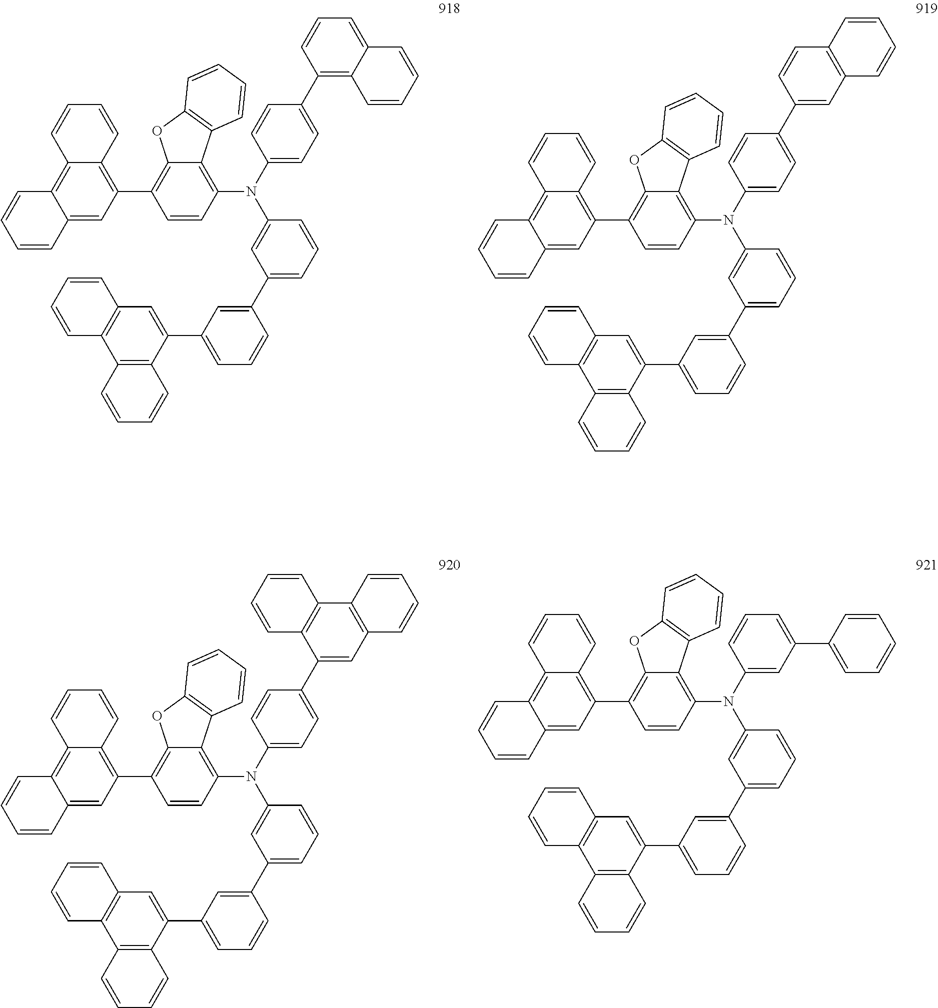 [SMILES: c1cc(-c2cccc(N(c3ccc(-c4cccc5ccccc45)cc3)c3ccc(-c4cc5ccccc5c5ccccc45)c4oc5ccccc5c34)c2)cc(-c2cc3ccccc3c3ccccc23)c1 ; c1cc(-c2cccc(N(c3ccc(-c4ccc5ccccc5c4)cc3)c3ccc(-c4cc5ccccc5c5ccccc45)c4oc5ccccc5c34)c2)cc(-c2cc3ccccc3c3ccccc23)c1 ; c1cc(-c2cccc(N(c3ccc(-c4cc5ccccc5c5ccccc45)cc3)c3ccc(-c4cc5ccccc5c5ccccc45)c4oc5ccccc5c34)c2)cc(-c2cc3ccccc3c3ccccc23)c1 ; c1ccc(-c2cccc(N(c3cccc(-c4cccc(-c5cc6ccccc6c6ccccc56)c4)c3)c3ccc(-c4cc5ccccc5c5ccccc45)c4oc5ccccc5c34)c2)cc1]

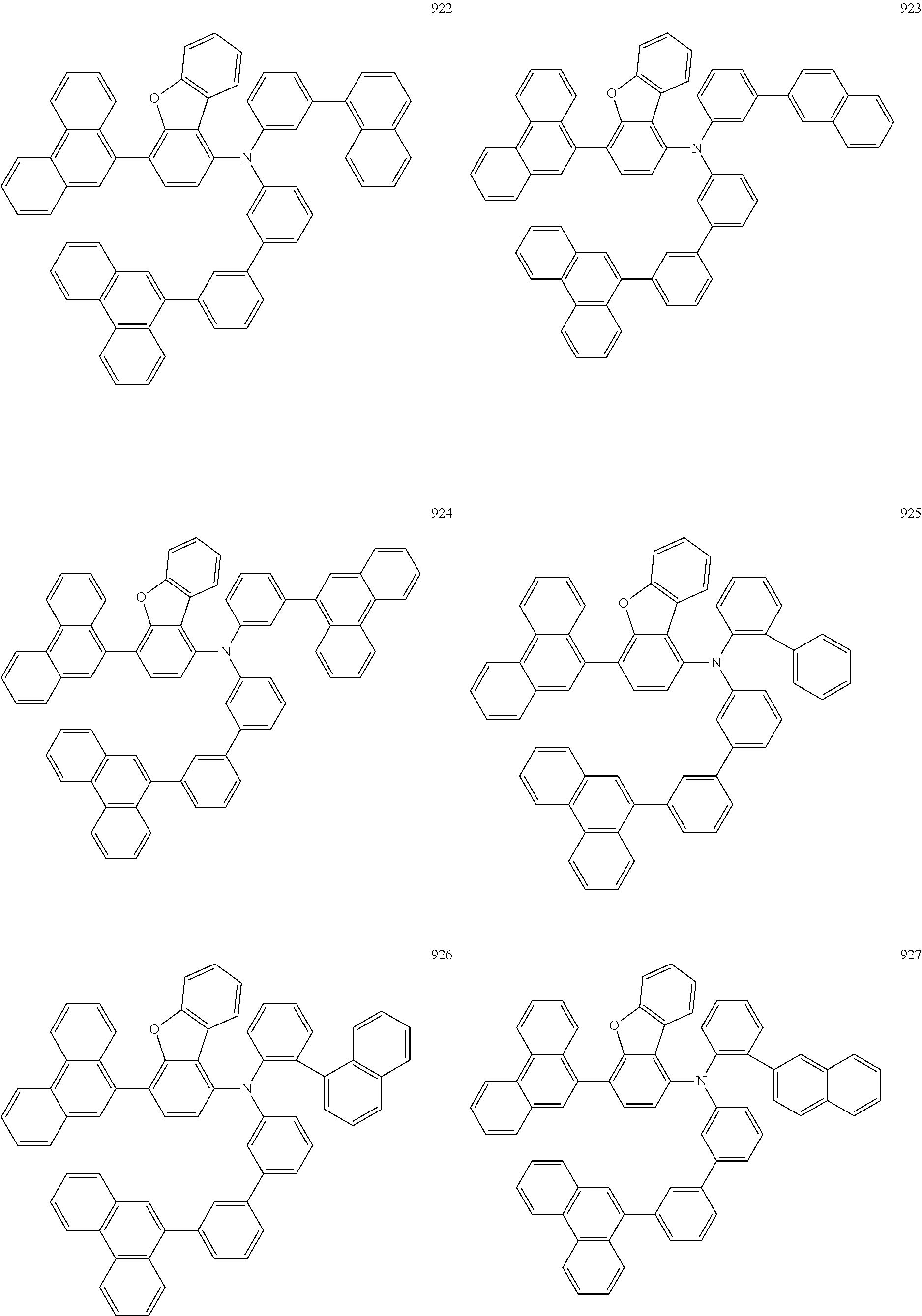

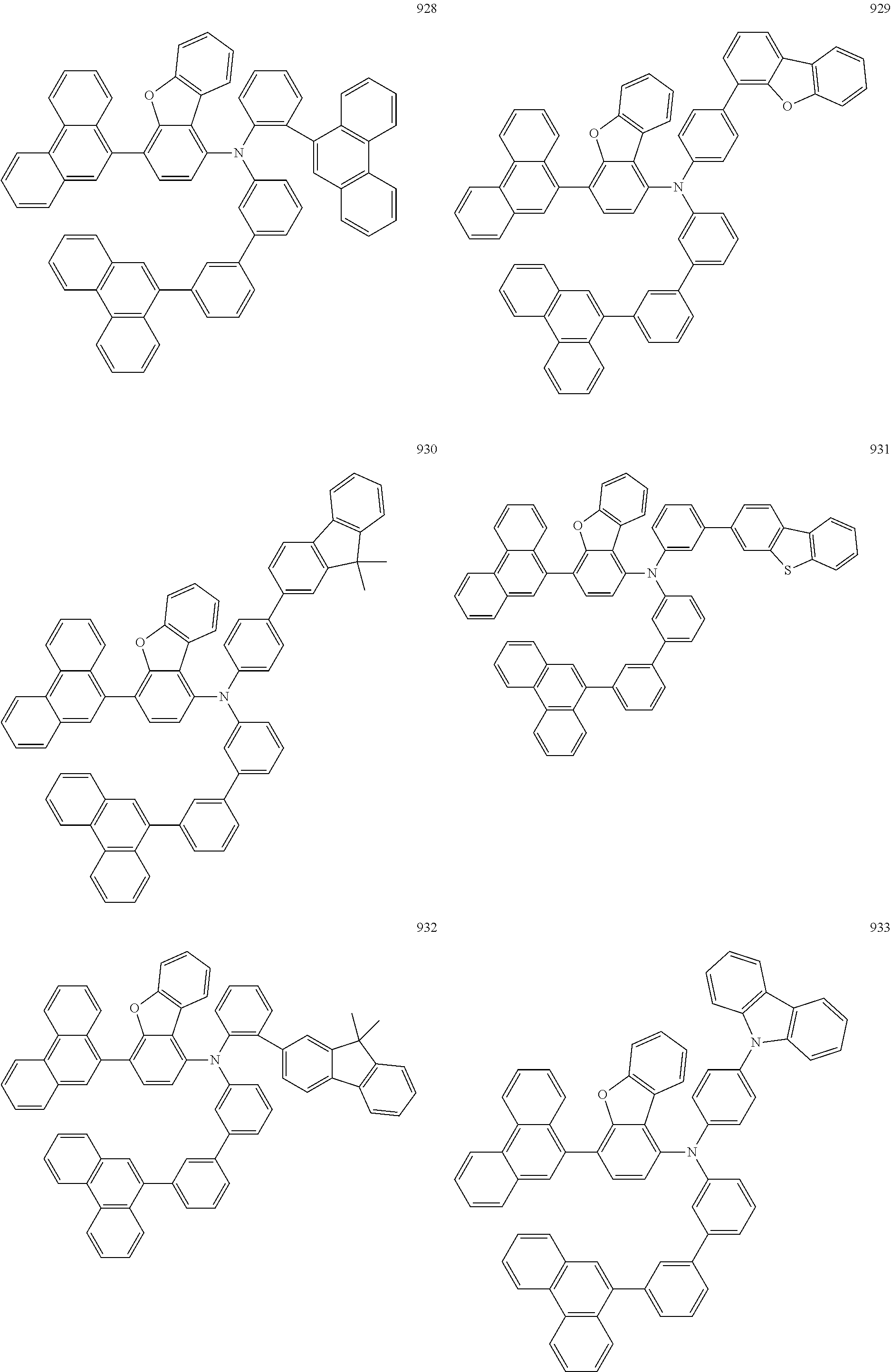

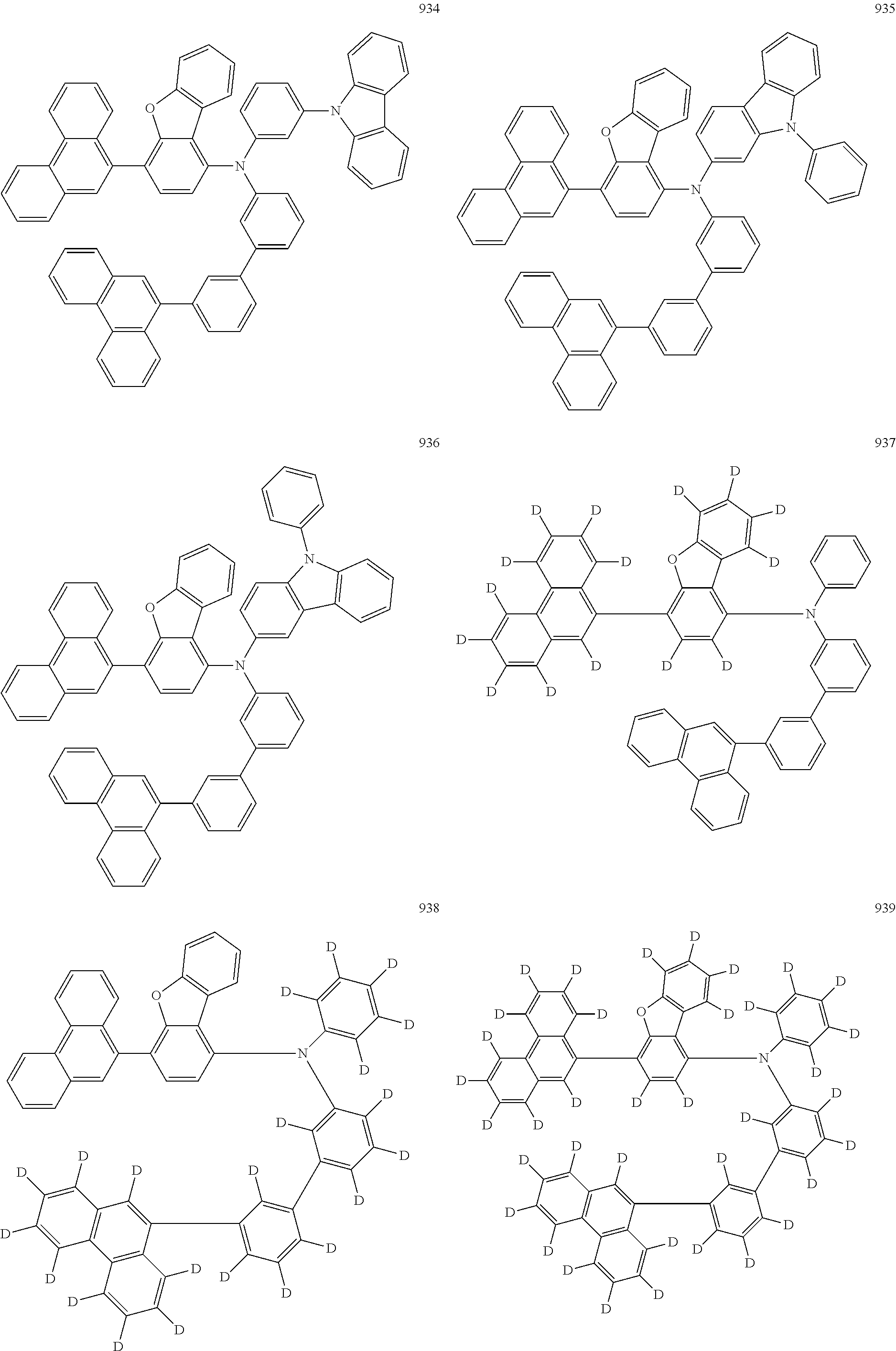

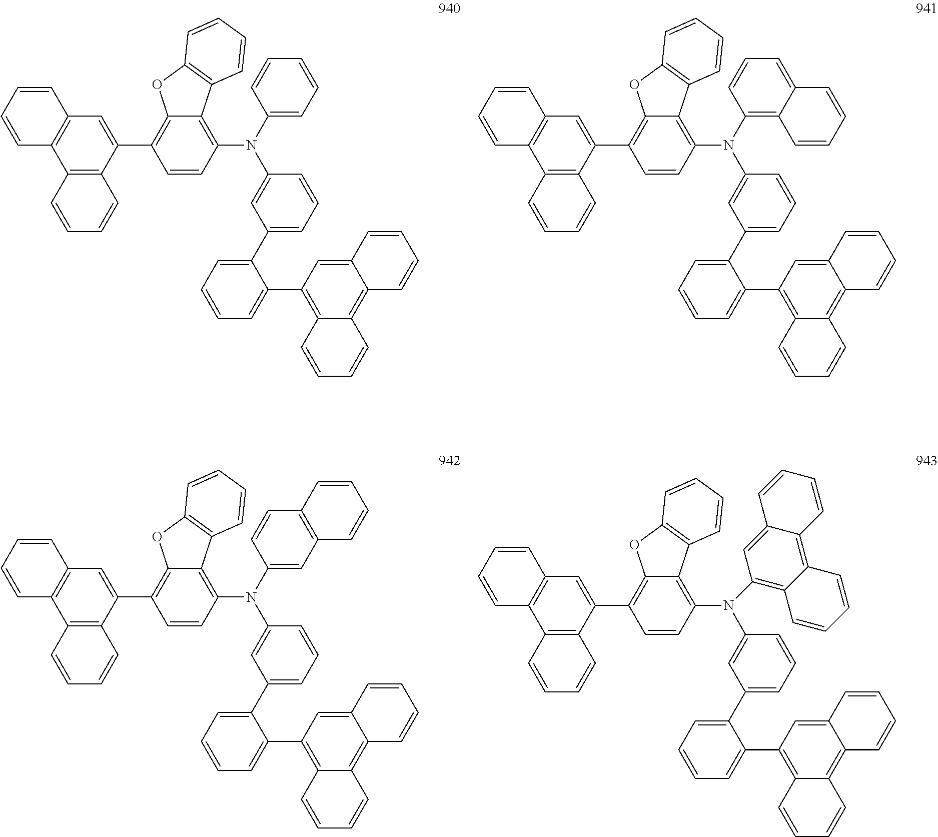 [SMILES: c1ccc(N(c2cccc(-c3ccccc3-c3cc4ccccc4c4ccccc34)c2)c2ccc(-c3cc4ccccc4c4ccccc34)c3oc4ccccc4c23)cc1 ; c1cc(-c2ccccc2-c2cc3ccccc3c3ccccc23)cc(N(c2cccc3ccccc23)c2ccc(-c3cc4ccccc4c4ccccc34)c3oc4ccccc4c23)c1 ; c1cc(-c2ccccc2-c2cc3ccccc3c3ccccc23)cc(N(c2ccc3ccccc3c2)c2ccc(-c3cc4ccccc4c4ccccc34)c3oc4ccccc4c23)c1 ; c1cc(-c2ccccc2-c2cc3ccccc3c3ccccc23)cc(N(c2cc3ccccc3c3ccccc23)c2ccc(-c3cc4ccccc4c4ccccc34)c3oc4ccccc4c23)c1]

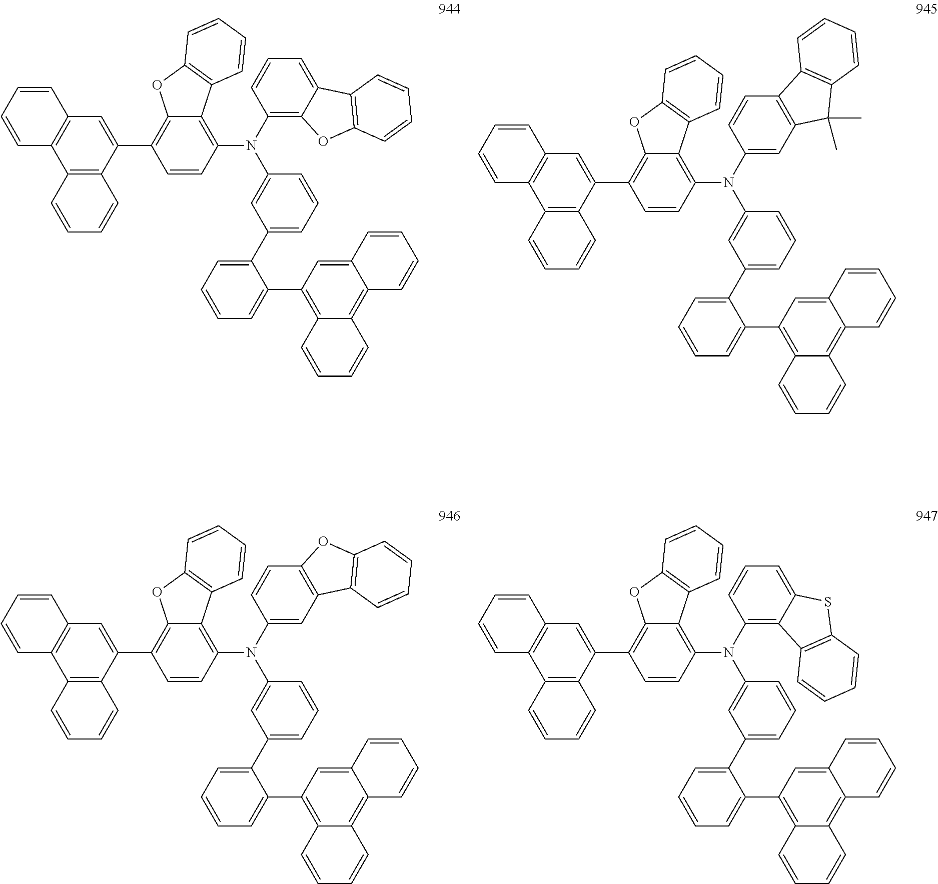 [SMILES: c1cc(-c2ccccc2-c2cc3ccccc3c3ccccc23)cc(N(c2cccc3c2oc2ccccc23)c2ccc(-c3cc4ccccc4c4ccccc34)c3oc4ccccc4c23)c1 ; CC1(C)c2ccccc2-c2ccc(N(c3cccc(-c4ccccc4-c4cc5ccccc5c5ccccc45)c3)c3ccc(-c4cc5ccccc5c5ccccc45)c4oc5ccccc5c34)cc21 ; c1cc(-c2ccccc2-c2cc3ccccc3c3ccccc23)cc(N(c2ccc3oc4ccccc4c3c2)c2ccc(-c3cc4ccccc4c4ccccc34)c3oc4ccccc4c23)c1 ; c1cc(-c2ccccc2-c2cc3ccccc3c3ccccc23)cc(N(c2cccc3sc4ccccc4c23)c2ccc(-c3cc4ccccc4c4ccccc34)c3oc4ccccc4c23)c1]

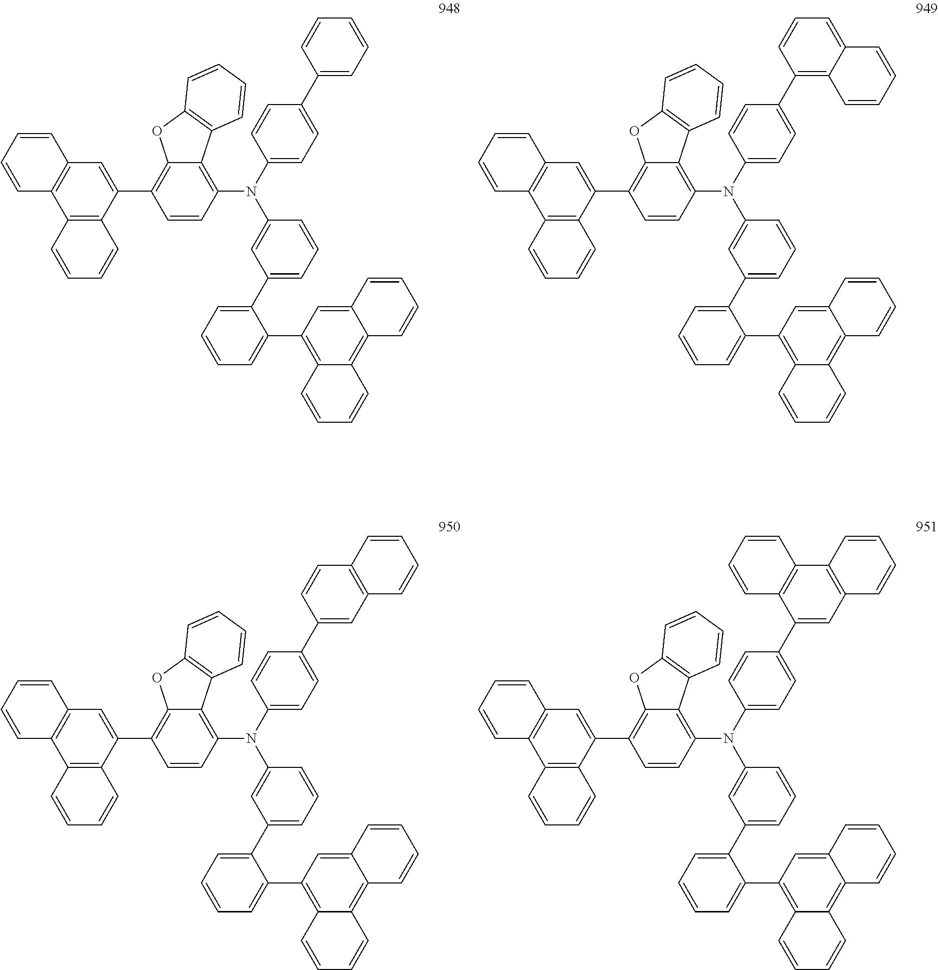 [SMILES: c1ccc(-c2ccc(N(c3cccc(-c4ccccc4-c4cc5ccccc5c5ccccc45)c3)c3ccc(-c4cc5ccccc5c5ccccc45)c4oc5ccccc5c34)cc2)cc1 ; c1cc(-c2ccccc2-c2cc3ccccc3c3ccccc23)cc(N(c2ccc(-c3cccc4ccccc34)cc2)c2ccc(-c3cc4ccccc4c4ccccc34)c3oc4ccccc4c23)c1 ; c1cc(-c2ccccc2-c2cc3ccccc3c3ccccc23)cc(N(c2ccc(-c3ccc4ccccc4c3)cc2)c2ccc(-c3cc4ccccc4c4ccccc34)c3oc4ccccc4c23)c1 ; c1cc(-c2ccccc2-c2cc3ccccc3c3ccccc23)cc(N(c2ccc(-c3cc4ccccc4c4ccccc34)cc2)c2ccc(-c3cc4ccccc4c4ccccc34)c3oc4ccccc4c23)c1]

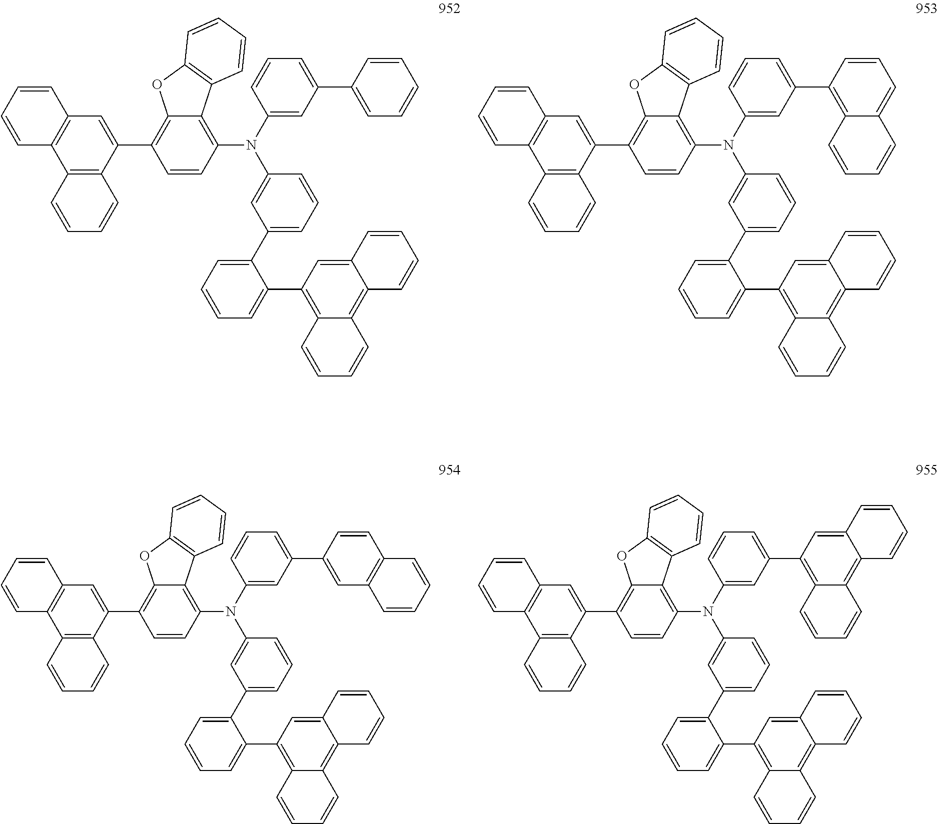 [SMILES: c1ccc(-c2cccc(N(c3cccc(-c4ccccc4-c4cc5ccccc5c5ccccc45)c3)c3ccc(-c4cc5ccccc5c5ccccc45)c4oc5ccccc5c34)c2)cc1 ; c1cc(-c2ccccc2-c2cc3ccccc3c3ccccc23)cc(N(c2cccc(-c3cccc4ccccc34)c2)c2ccc(-c3cc4ccccc4c4ccccc34)c3oc4ccccc4c23)c1 ; c1cc(-c2ccc3ccccc3c2)cc(N(c2cccc(-c3ccccc3-c3cc4ccccc4c4ccccc34)c2)c2ccc(-c3cc4ccccc4c4ccccc34)c3oc4ccccc4c23)c1 ; c1cc(-c2ccccc2-c2cc3ccccc3c3ccccc23)cc(N(c2cccc(-c3cc4ccccc4c4ccccc34)c2)c2ccc(-c3cc4ccccc4c4ccccc34)c3oc4ccccc4c23)c1]

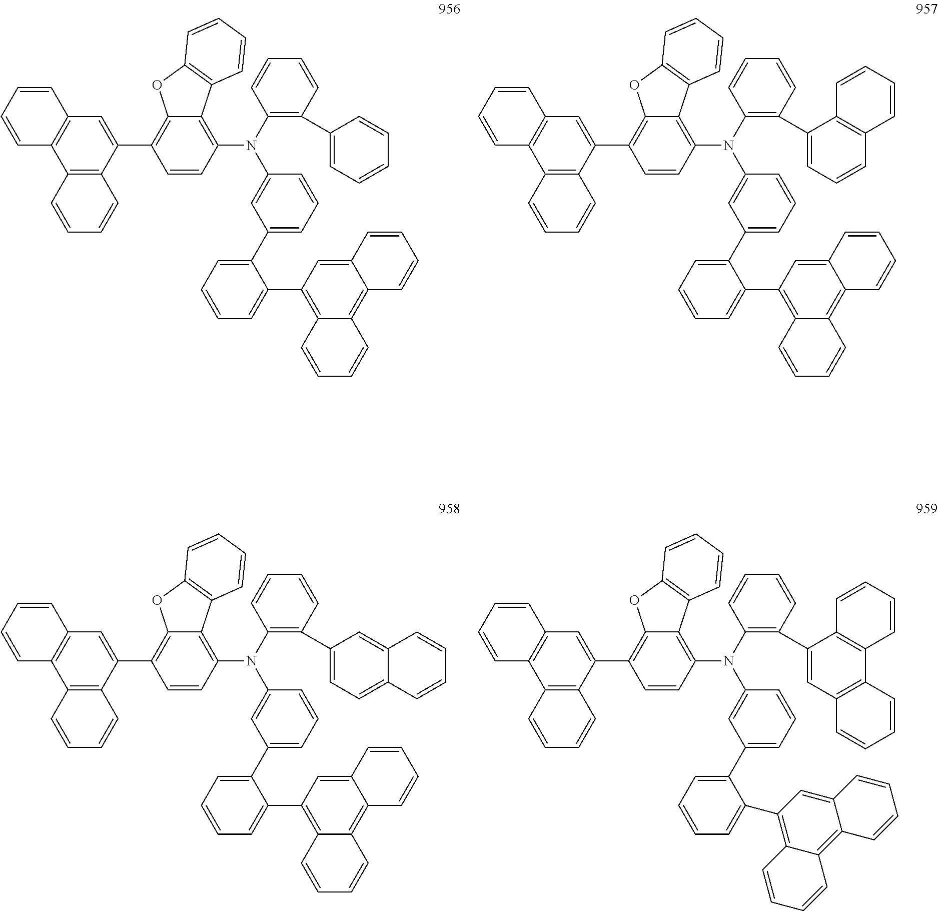

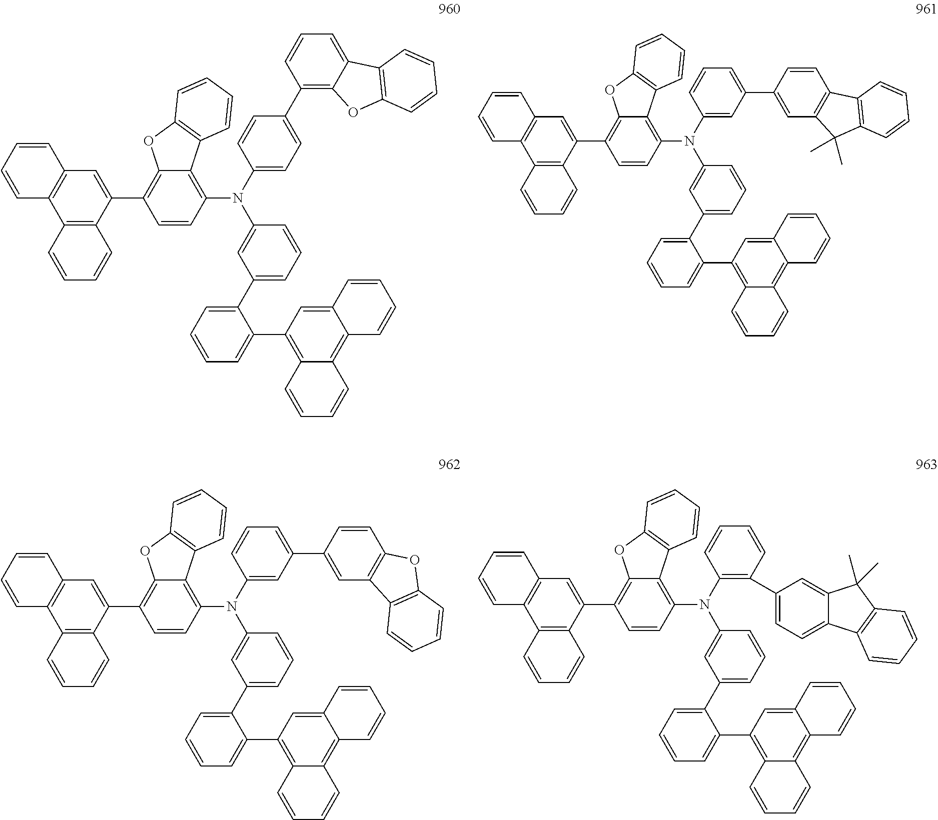 [SMILES: c1cc(-c2ccccc2-c2cc3ccccc3c3ccccc23)cc(N(c2ccc(-c3cccc4c3oc3ccccc34)cc2)c2ccc(-c3cc4ccccc4c4ccccc34)c3oc4ccccc4c23)c1 ; CC1(C)c2ccccc2-c2ccc(-c3cccc(N(c4cccc(-c5ccccc5-c5cc6ccccc6c6ccccc56)c4)c4ccc(-c5cc6ccccc6c6ccccc56)c5oc6ccccc6c45)c3)cc21 ; c1cc(-c2ccc3oc4ccccc4c3c2)cc(N(c2cccc(-c3ccccc3-c3cc4ccccc4c4ccccc34)c2)c2ccc(-c3cc4ccccc4c4ccccc34)c3oc4ccccc4c23)c1 ; CC1(C)c2ccccc2-c2ccc(-c3ccccc3N(c3cccc(-c4ccccc4-c4cc5ccccc5c5ccccc45)c3)c3ccc(-c4cc5ccccc5c5ccccc45)c4oc5ccccc5c34)cc21]

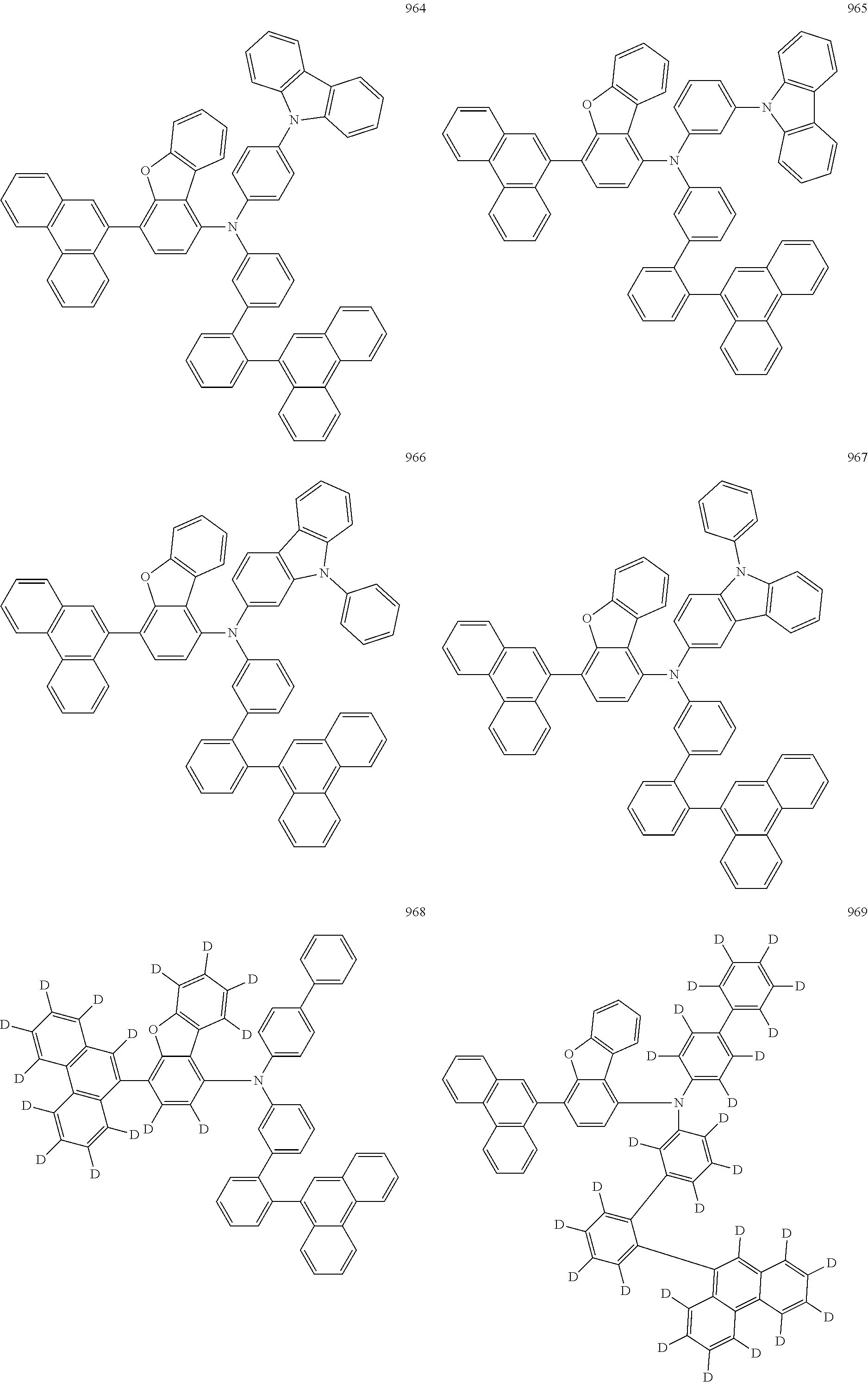

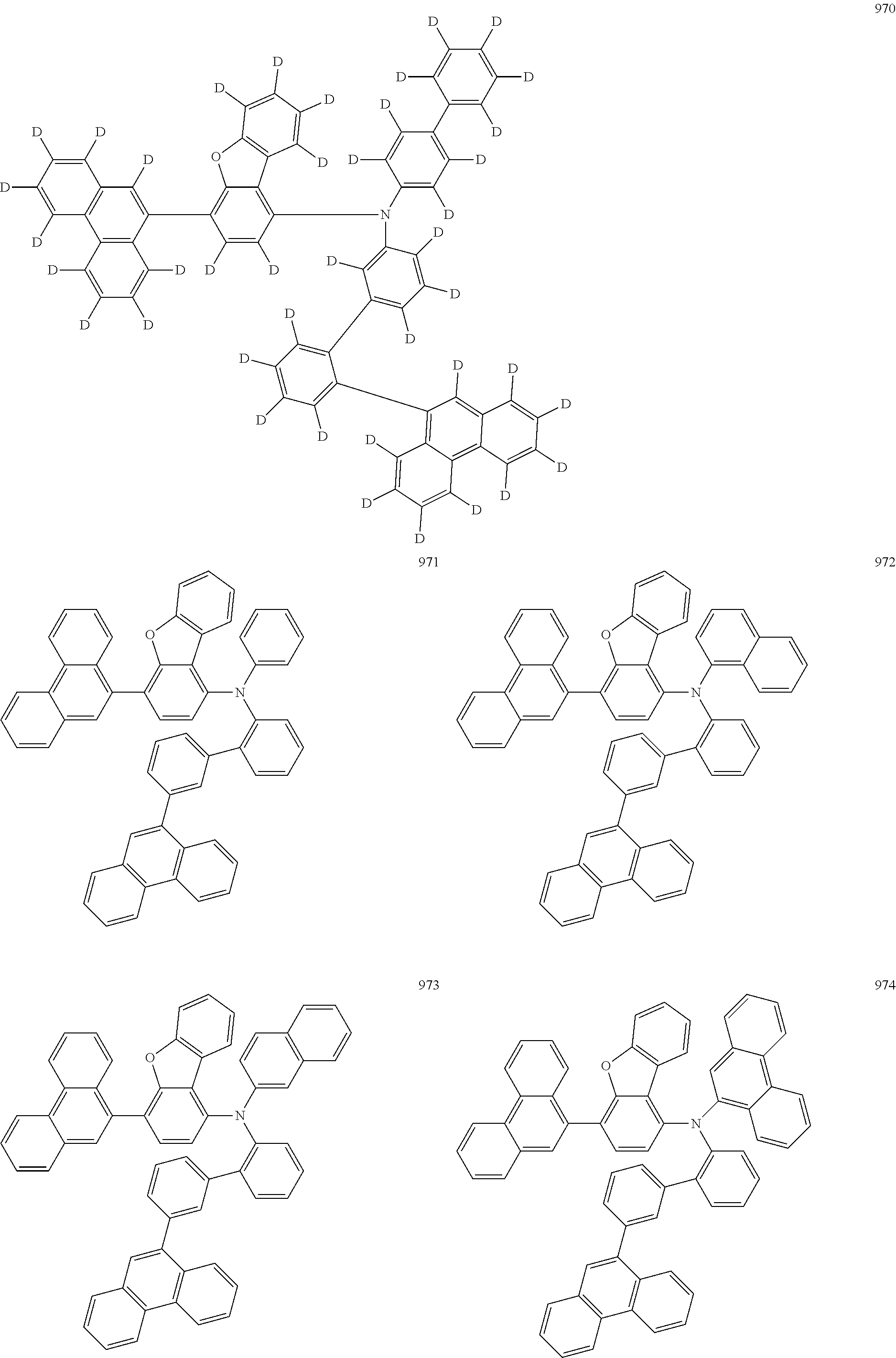

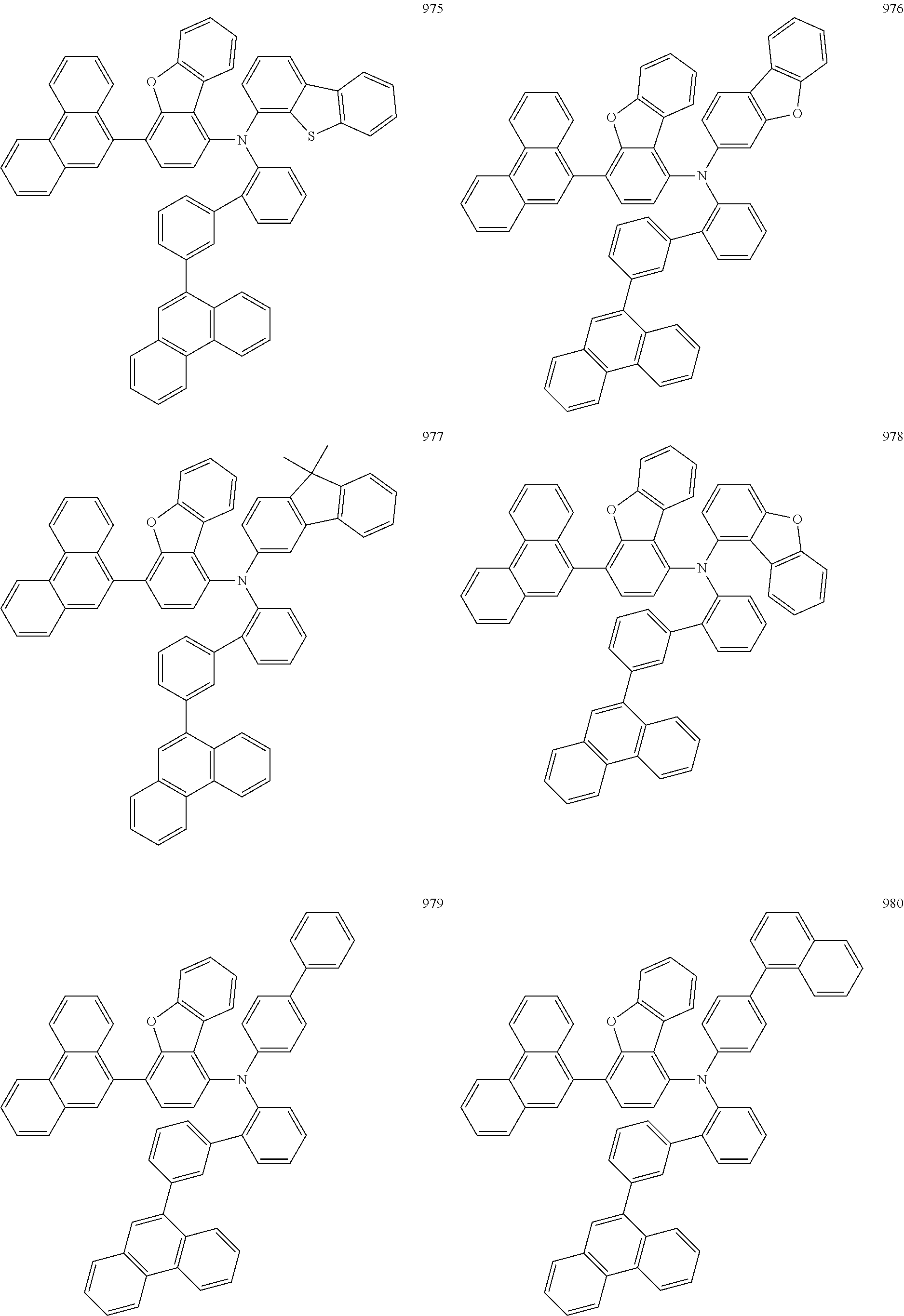

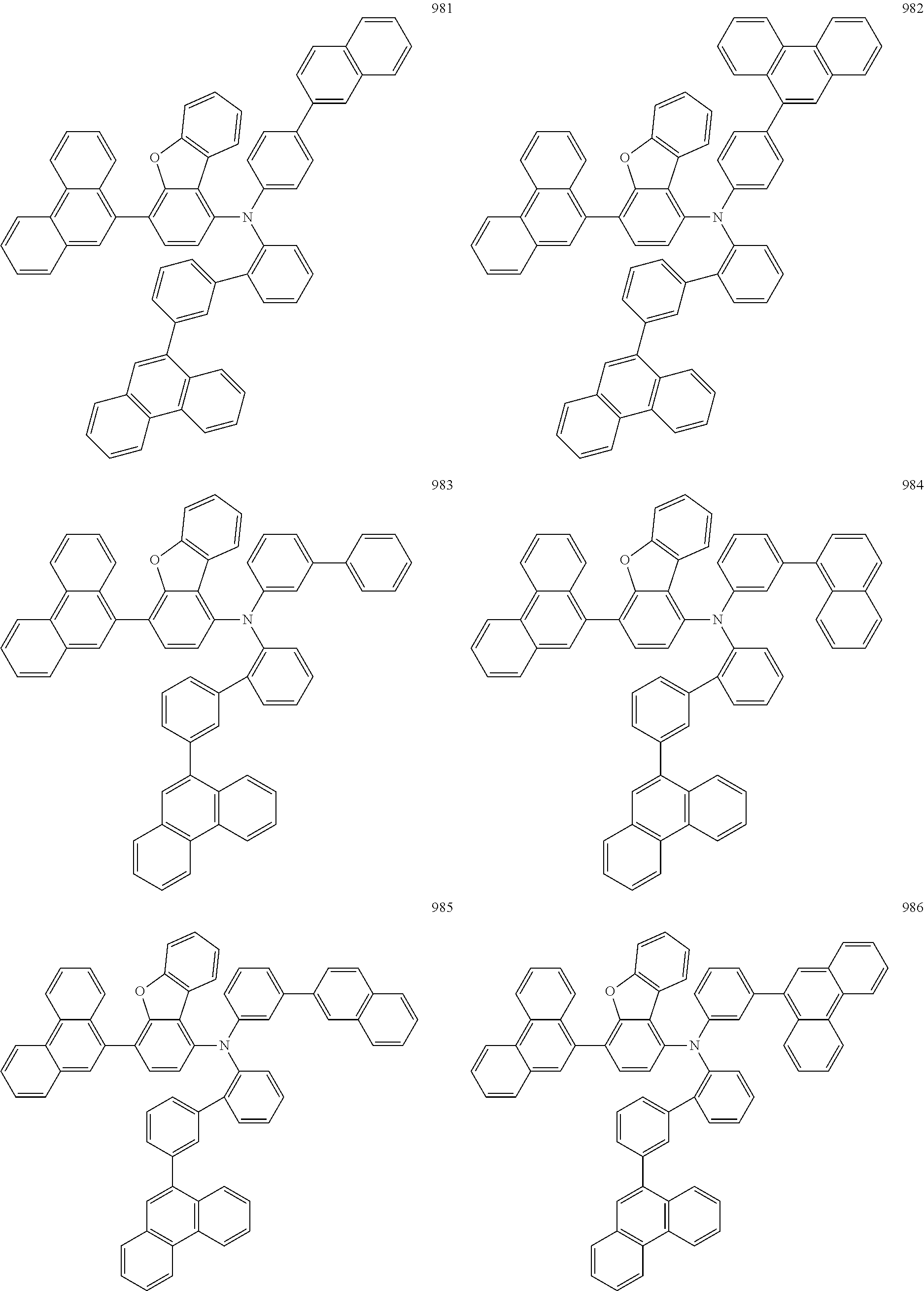

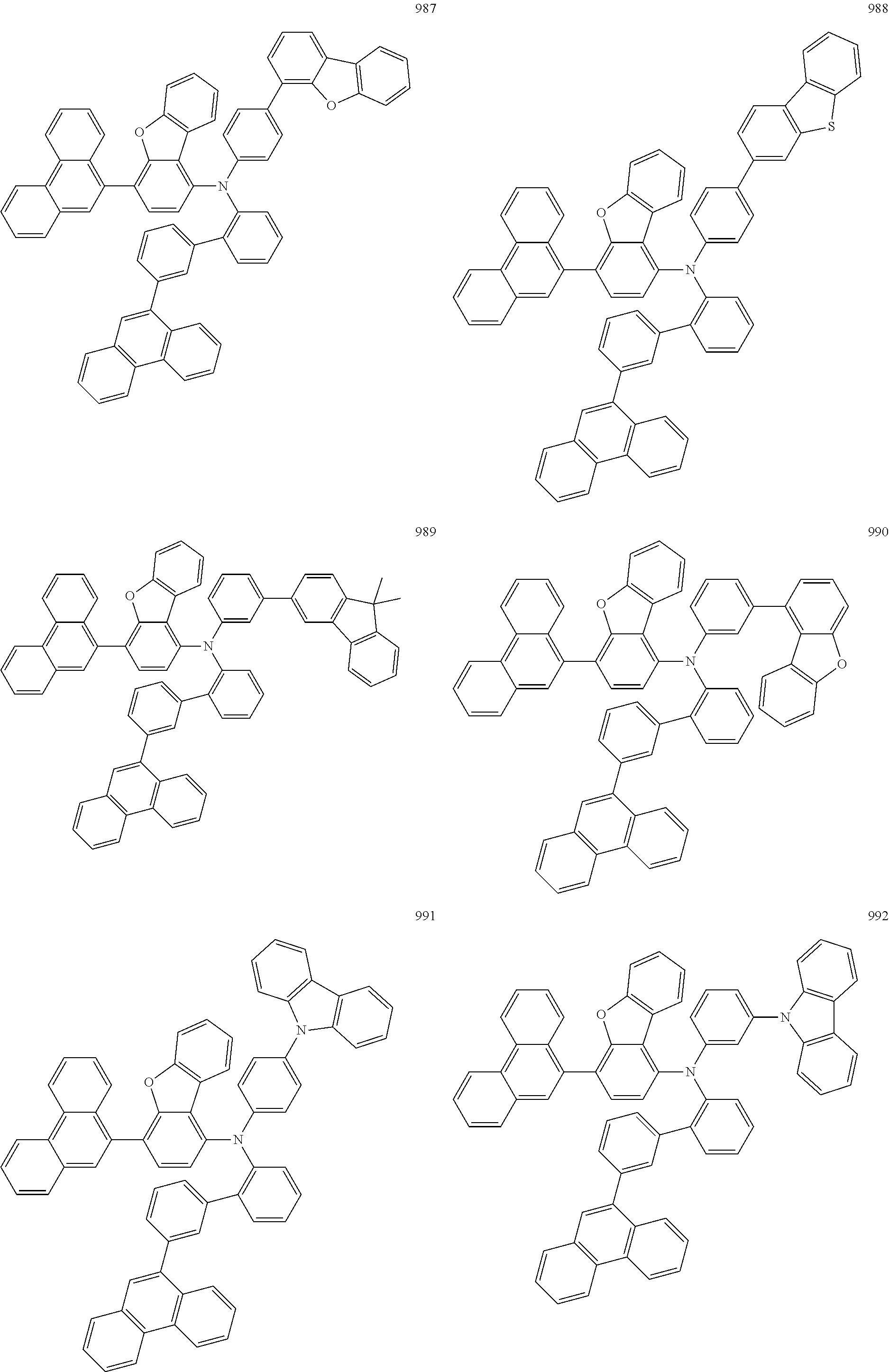

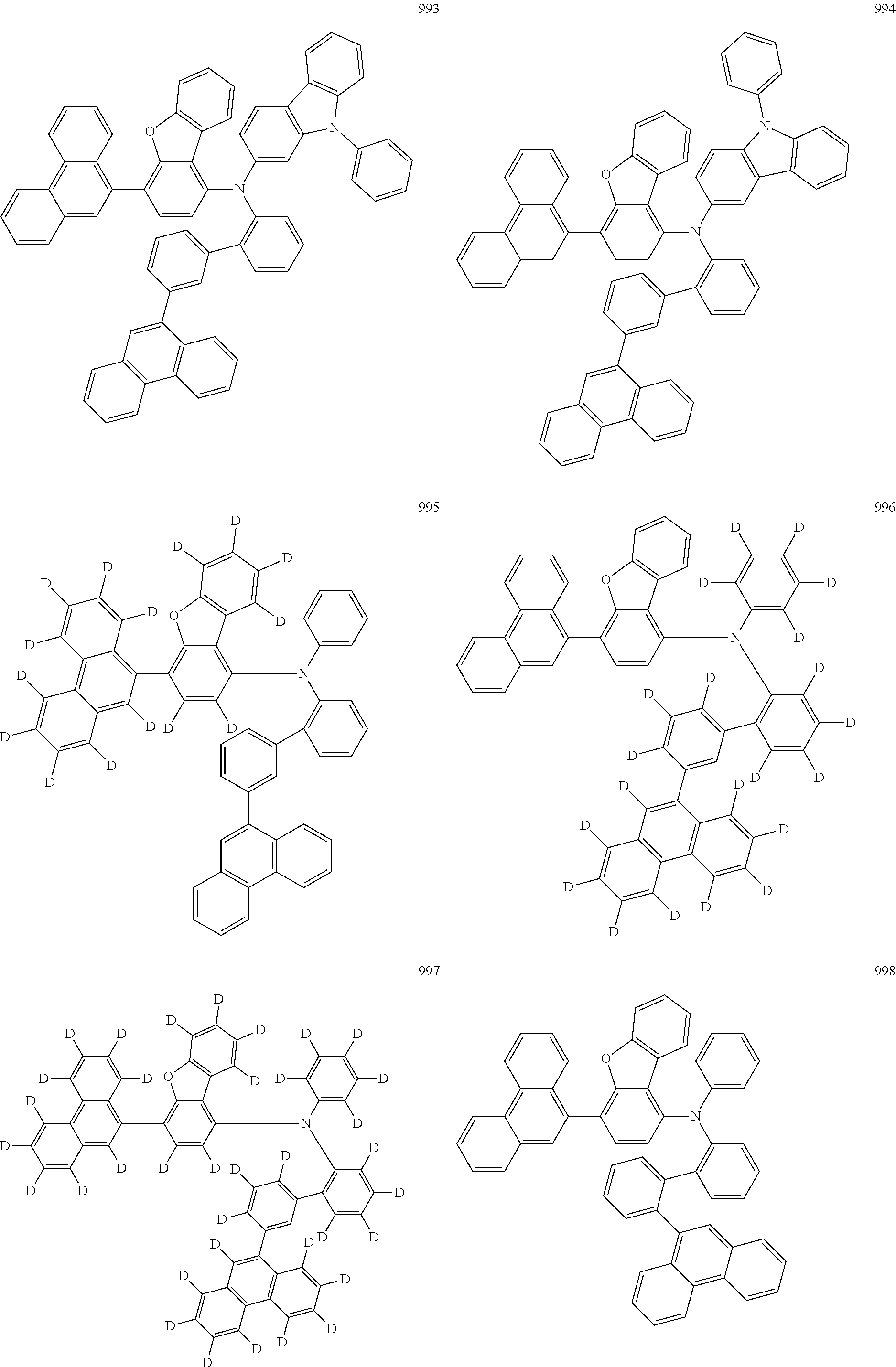

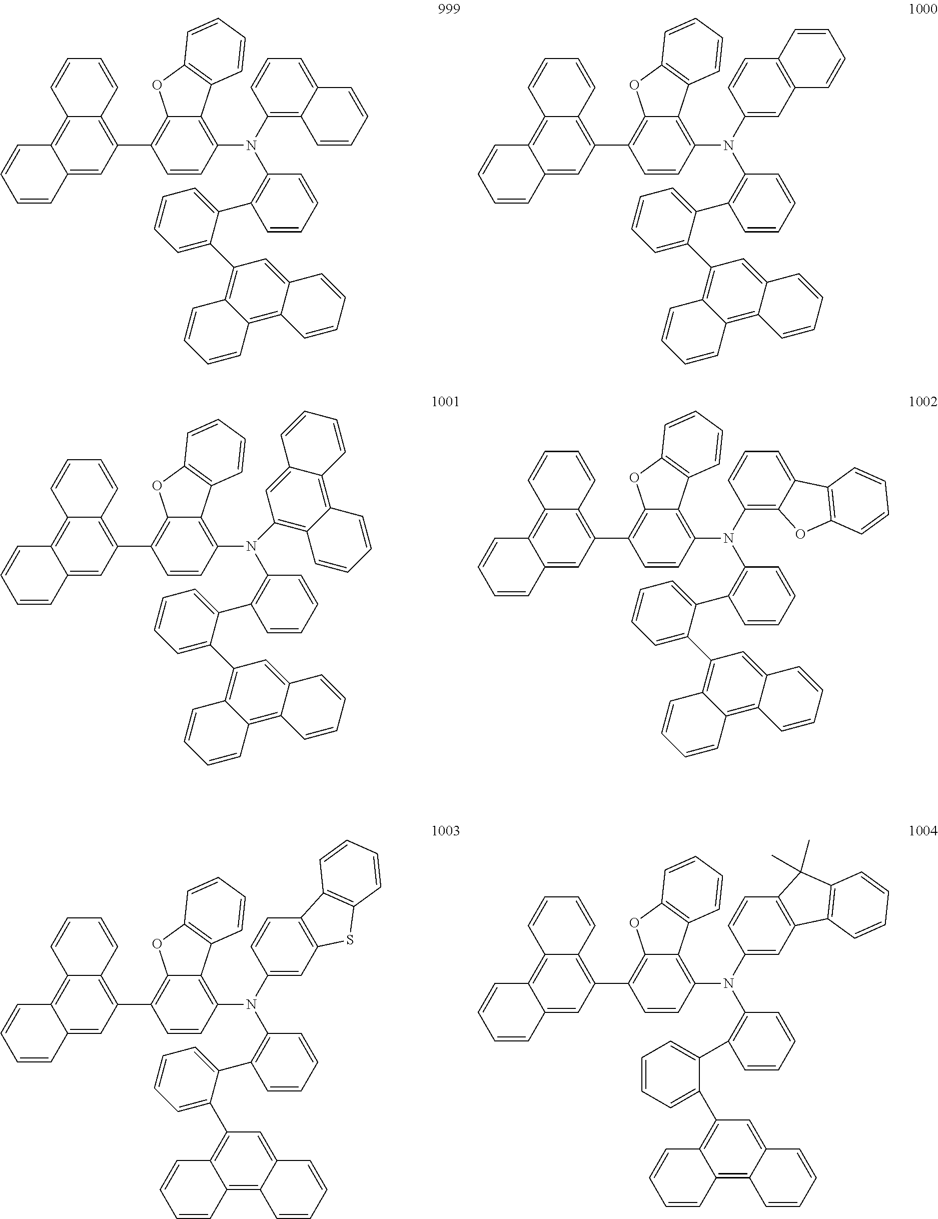

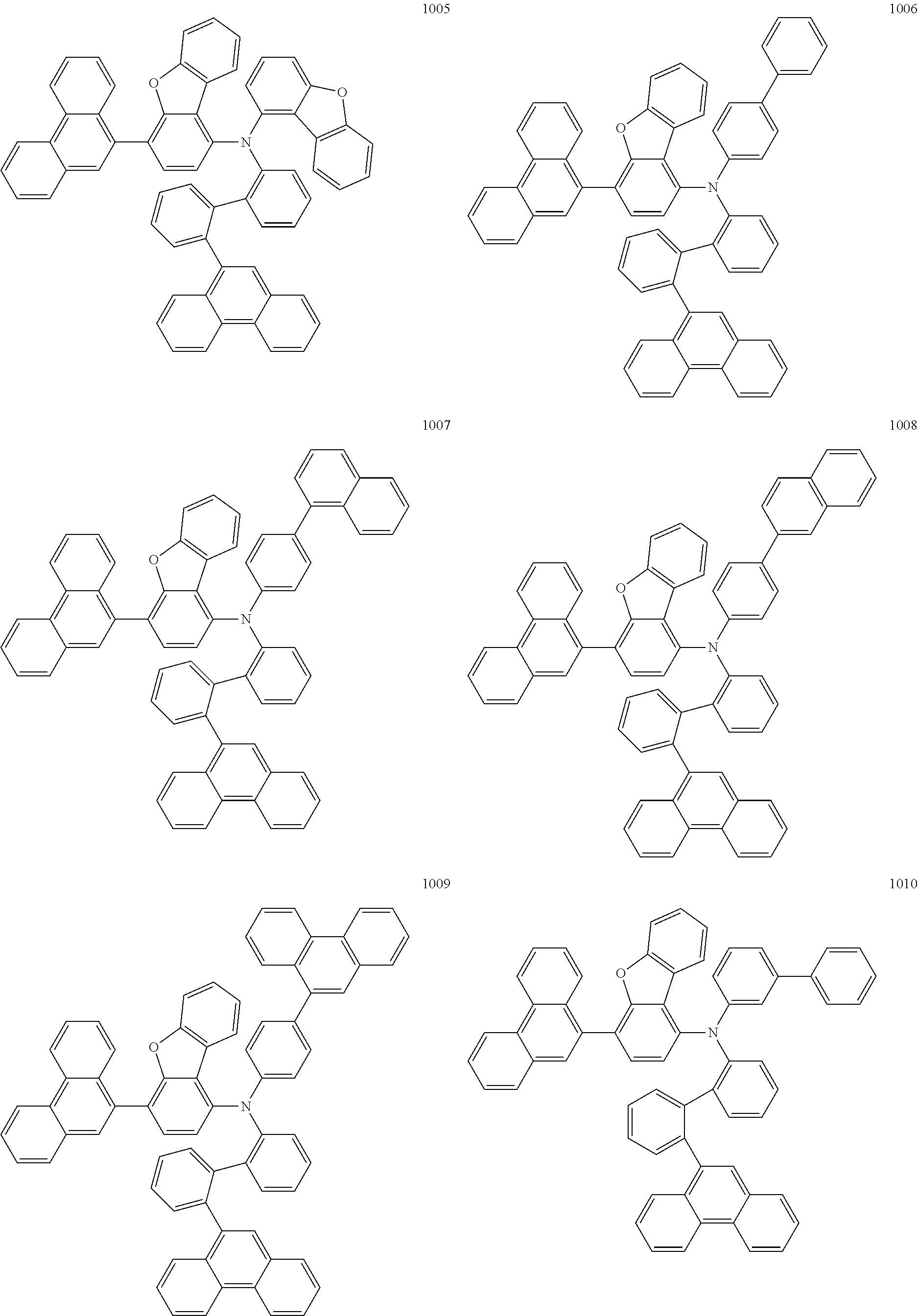

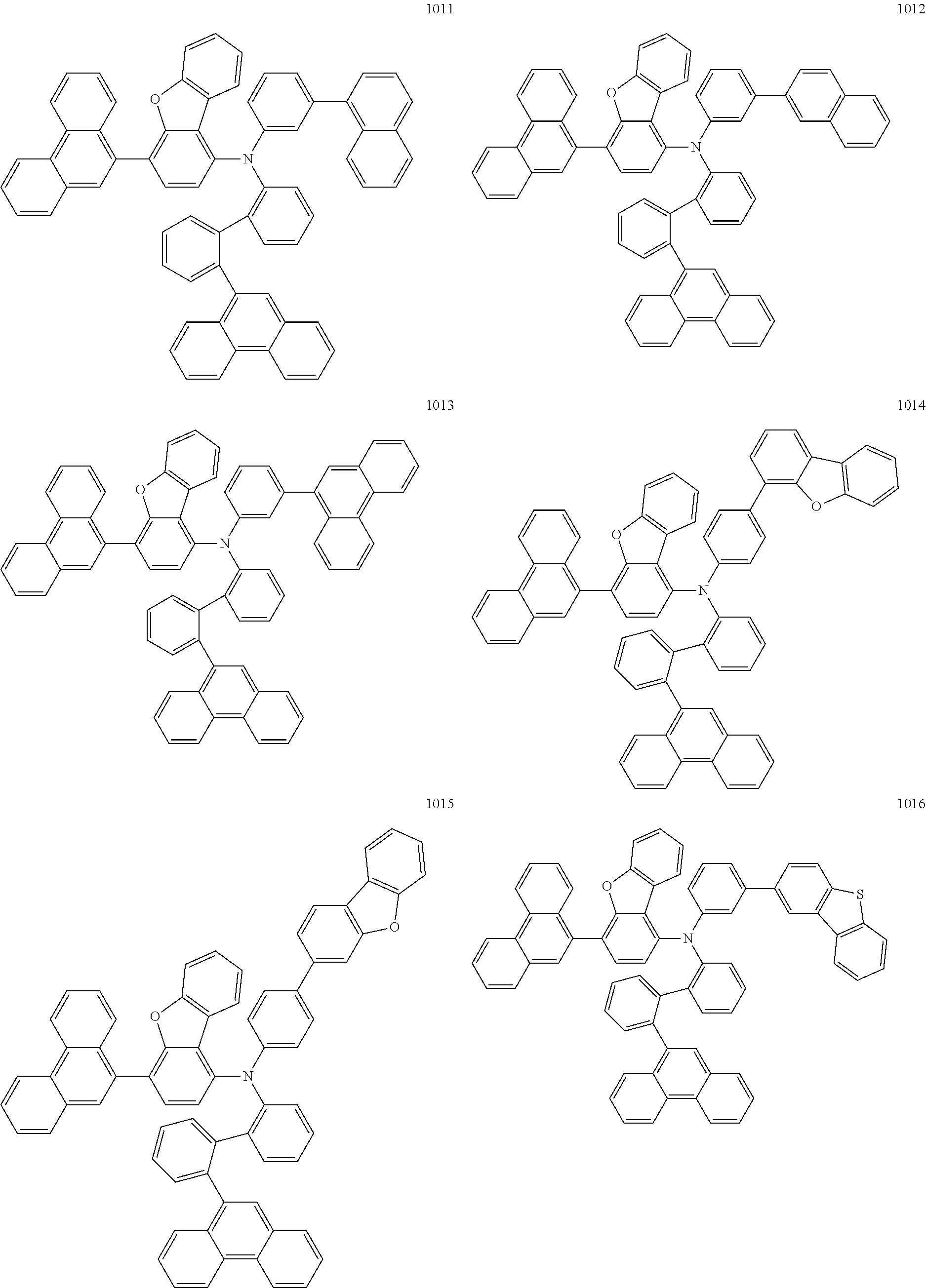

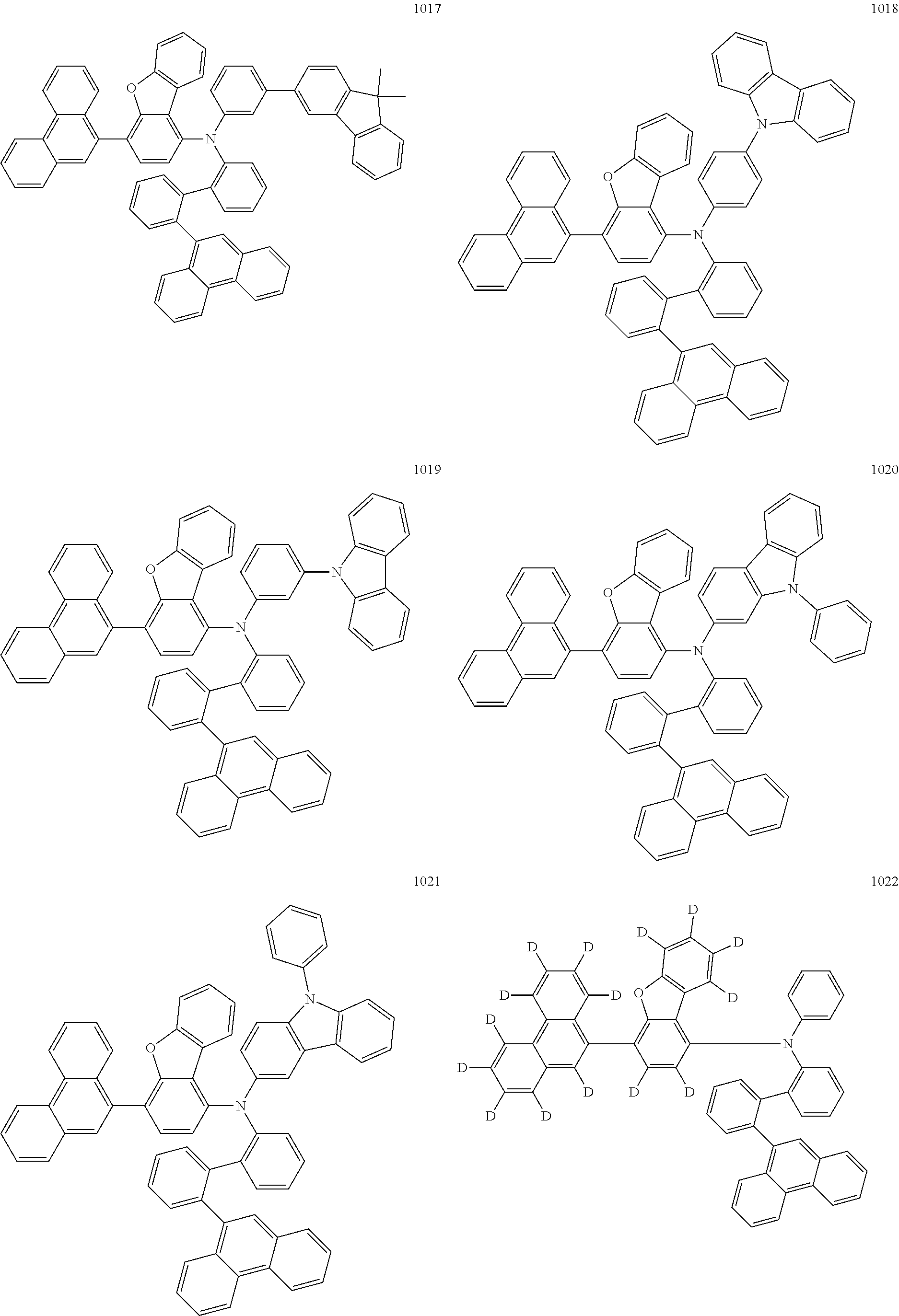

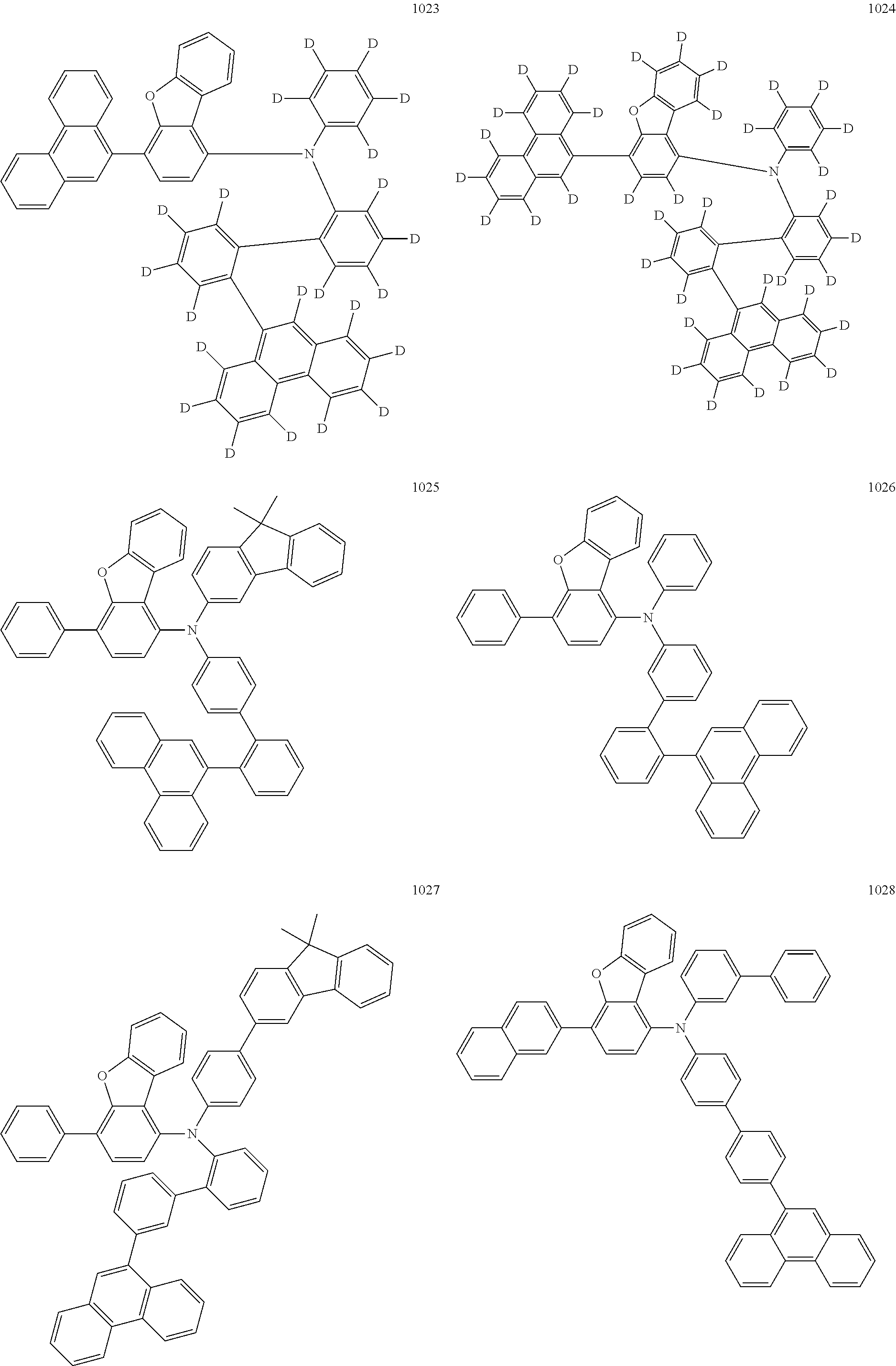

An organic light emitting diode according to an embodiment of the present disclosure may include a first electrode (anode), a second electrode (cathode) facing the first electrode, and one or more organic layers disposed between the first electrode and the second electrode.

At least one of the one or more organic layers may comprise an organic compound represented by Chemical Formula 1.

The organic layer may include one or more of hole injection layer (HIL), a hole transport layer (HTL), a hole transport auxiliary layer, an emitting layer (EML), an electron transport auxiliary layer, an electron transport layer (ETL), and an electron injection layer (EIL).

For example, the organic light emitting diode may have a structure in which a first electrode, a hole injection layer (HIL), a hole transport layer (HTL), a hole transport auxiliary layer, an emitting layer (EML), an electron transport layer (ETL), an electron injection layer (EIL), and a second electrode are sequentially stacked.

Here, the organic layer including the organic compound represented by Chemical Formula 1 according to an embodiment of the present disclosure may be a hole transport layer (HTL) or a hole transport auxiliary layer.

The one or more organic layers may further include one or more selected from the group consisting of a hole injection layer, an emitting layer, an electron transport auxiliary layer, an electron transport layer, and an electron injection layer.

For example, when the organic compound represented by Chemical Formula 1 is used as a material for the hole transport auxiliary layer, it may have an energy level suitable for serving as a hole transport auxiliary layer that transfers holes from the hole transport layer to the emitting layer and blocks electrons coming from the emitting layer.

The organic light emitting diode according to an embodiment of the present disclosure may excellently realize a target color coordinates of the emitting layer, even when a hole transport layer and/or a hole transport auxiliary layer including the organic compound represented by Chemical Formula 1 is combined with an emitting layer of any color.

The first electrode may be an anode, and may include a material having excellent transparency and conductivity, such as indium tin oxide (ITO), indium zinc oxide (IZO), tin oxide (SnO₂), or zinc oxide (ZnO).

The second electrode may be a cathode, and may include a material such as lithium (Li), aluminum (Al), aluminum-lithium (Al—Li), calcium (Ca), magnesium (Mg), magnesium-indium (Mg—In), or magnesium-silver (Mg—Ag). In addition, for a top-emission organic light emitting diode, a transparent second electrode through which can transmit light may be formed using indium tin oxide (ITO) or indium zinc oxide (IZO).

A capping layer (CPL) may be formed on the surface of the second electrode by a composition for forming a capping layer.

In addition, an encapsulation layer (or protecting layer) may be additionally disposed on the capping layer to protect the organic light emitting diode from moisture and oxygen. The encapsulation layer (or protective layer) may be formed of a curable adhesive composition containing an inorganic desiccant.

The compound for the hole injection layer or the hole transport layer is not particularly limited, and may be any compound, as long as it is conventionally used for the hole injection layer or the hole transport layer. Non-limiting examples of compounds for the hole injection layer or hole transport layer may include phthalocyanine derivatives, porphyrin derivatives, triarylamine derivatives, indolocarbazole derivatives, etc. Examples thereof may include 1,4,5,8,9,11-hexaazatriphenylene-hexacarbonitrile (HAT-CN), copper phthalocyanine (CuPc), 4,4′,4″-tris(3-methylphenylamino)triphenylamine (m-MTDATA), 4,4′,4″-tris(3-methylphenylamino)phenoxybenzene (m-MTDAPB), 4,4′,4″-tri(N-carbazolyl)triphenylamine (TCTA), 4,4′,4″-tris(N-(2-naphthyl)-N-phenylamino)triphenylamine (2-TNATA), N4,N4,N4′,N4′-tetra([1,1′-biphenyl]-4-yl)-[1,1′-biphenyl]-4,4′-diamine, bis(N-(1-naphthyl)-N-phenyl)benzidine (α-NPD), N,N′-di(naphthalen-1-yl)-N,N′-biphenyl-benzidine (NPB), N,N′-biphenyl-N,N′-bis(3-methylphenyl)-1,1′-biphenyl-4,4′-diamine (TPD), etc.

The compound included in the emitting layer is not particularly limited, and may be any compound, as long as it is conventionally used for the emitting layer. A single light emitting compound or a light emitting host compound may be used.

The light emitting compound in the emitting layer may include, but is not limited to, a compound capable of emitting light through phosphorescence, fluorescence, thermally activated delayed fluorescence (TADF, also referred to as E-type delayed fluorescence), triplet-triplet annihilation, or a combination of these processes. The light emitting compound may be selected from a variety of materials depending on the desired emission color. Non-limiting examples of the light emitting compounds include fused ring derivatives such as phenanthrene, anthracene, pyrene, tetracene, pentacene, perylene, naphthopyrene, dibenzopyrene, rubrene, and chrysene, benzoxazole derivatives, benzothiazole derivatives, benzimidazole derivatives, benzotriazole derivatives, oxazole derivatives, oxadiazole derivatives, thiazole derivatives, imidazole derivatives, thiadiazole derivatives, triazole derivatives, pyrazoline derivatives, stilbene derivatives, thiophene derivatives, tetraphenylbutadiene derivatives, cyclopentadiene derivatives, bis(styryl) derivatives, bis(styryl)arylene derivatives, diazaindacene derivatives, furan derivatives; benzofuran derivatives, isobenzofuran derivatives, dibenzofuran derivatives, coumarin derivatives, dicyanomethylenepyran derivatives, dicyanomethylenethiopyran derivatives, polymethine derivatives, cyanine derivatives, oxobenzoanthracene derivatives, xanthene derivatives, rhodamine derivatives, fluorescein derivatives, pyrylium derivatives, carbostyril derivatives, acridine derivatives, oxazine derivatives, phenylene oxide derivatives, quinacridone derivatives, quinazoline derivatives, pyrrolopyridine derivatives, furopyridine derivatives, 1,2,5-thiadiazolopyrene derivatives, pyromethene derivatives, perinone derivatives, pyrrolopyrrole derivatives, squarylium derivatives, violanthrone derivatives, phenazine derivatives, acridone derivatives, deazaflavin derivatives, fluorene derivatives, benzofluorene derivatives, aromatic boron derivatives, aromatic nitrogen boron derivatives, and metal complexes (e.g., complexes of metals such as Ir, Pt, Au, Eu, Ru, Re, Ag, and Cu with heteroaromatic ring ligands). Examples thereof include N1,N1,N6,N6-tetrakis(4-(1-silyl)phenyl)pyrene-1,6-diamine, 2,12-di-tert-butyl-5,9-bis(4-(tert-butyl)phenyl)-7-(3,5-di-tert-butylphenyl)-5,9-dihydro-5,9-diaza-13b-boranaphtho[3,2,1-de]anthracene (t-DABNA-dtB), platinum octaethylporphyrin (PtOEP), Ir(ppy)₃, Ir(ppy)₂(acac), Ir(mppy)₃, Ir(PPy)₂(m-bppy), Btplr(acac), Ir(btp)₂(acac), Ir(2-phq)₃, Hex-Ir(phq)₃, Ir(fbi)₂(acac), fac-tris(2-(3-p-xylyl)phenyl)pyridine iridium(III), Eu(dbm)₃(Phen), Ir(piq)₃, Ir(piq)₂(acac), Ir(Fliq)₂(acac), Ir(Flq)₂(acac), Ru(dtb-bpy)₃·2(PF₆), Ir(BT)₂(acac), Ir(DMP)₃, Ir(Mphq)₃, Ir(phq)₂tpy, fac-Ir(ppy)₂Pc, Ir(dp)PQ₂, Ir(Dpm)(Piq)₂, Hex-Ir(piq)₂(acac), Hex-Ir(piq)₃, Ir(dmpq)₃, Ir(dmpq)₂(acac), FPQIrpic, FIrpic, etc.

As the host compound in the emitting layer, an emissive host, a hole-transporting host, an electron-transporting host, or a combination thereof may be used. Non-limiting examples of emissive host compounds include fused ring derivatives such as anthracene or pyrene; bis(styryl) derivatives such as bis(styryl)anthracene or di(styryl)benzene derivatives; tetraphenylbutadiene derivatives; cyclopentadiene derivatives; fluorene derivatives; benzofluorene derivatives; N-phenylcarbazole (9-phenylcarbazole) derivatives; and carbazolyl nitrile derivatives. Non-limiting examples of hole-transporting host materials include carbazole derivatives, dibenzofuran derivatives, dibenzothiophene derivatives, triarylamine derivatives, indolocarbazole derivatives, and benzoxazinophenoxazine derivatives. Non-limiting examples of electron-transporting host materials include pyridine derivatives, triazine derivatives, phosphine oxide derivatives, benzofuropyridine derivatives, and dibenzooxasiline derivatives. Examples thereof include 9,10-bis(2-naphthyl)anthracene (ADN), tris(8-hydroxyquinolinato)aluminum (Alq₃), BAlq (beryllium 8-hydroxyquinolinate), DPVBi (4,4′-bis(2,2-biphenylethenyl)-1,1′-biphenyl) series, spiro-DPVBi (spiro-4,4′-bis(2,2-biphenylethenyl)-1,1′-biphenyl), LiPBO (2-(2-benzoxazolyl)phenol lithium salt), bis(biphenylvinyl)benzene, aluminum-quinoline metal complexes, and metal complexes of imidazole, thiazole, and oxazole.

The compound for the electron injection layer or the electron transport layer is not particularly limited, and may be any compound, as long as it is conventionally used for the electron injection layer or the electron transport layer. Non-limiting examples of the compound for the electron injection layer or the electron transport layer include pyridine derivatives, naphthalene derivatives, anthracene derivatives, phenanthroline derivatives, perinone derivatives, coumarin derivatives, naphthalimide derivatives, anthraquinone derivatives, diphenoquinone derivatives, diphenylquinone derivatives, perylene derivatives, oxadiazole derivatives, thiophene derivatives, triazole derivatives, thiadiazole derivatives, metal complexes of oxine derivatives, quinolinol-based metal complexes, quinoxaline derivatives and polymers thereof, benzazole compounds, gallium complexes, pyrazole derivatives, perfluorinated phenylene derivatives, triazine derivatives, pyrazine derivatives, benzoquinoline derivatives, imidazopyridine derivatives, borane derivatives, benzimidazole derivatives, benzoxazole derivatives, benzothiazole derivatives, quinoline derivatives, oligopyridine derivatives such as terpyridine, bipyridine derivatives, terpyridine derivatives, naphthyridine derivatives, aldazine derivatives, carbazole derivatives, indole derivatives, phosphine oxide derivatives, bis(styryl) derivatives, quinolinol-based metal complexes, hydroxazole-based metal complexes, azomethine-based metal complexes, tropolone-based metal complexes, flavonol-based metal complexes, benzoquinoline-based metal complexes, and metal salts. These materials may be used alone or in combination with other materials. Examples thereof include 2-(4-(9,10-di(naphthalen-2-yl)anthracen-2-yl)phenyl)-1-phenyl-1H-benzo[d]imidazole, tris(8-hydroxyquinolinato)aluminum (Alq₃), LiF, Liq, Li₂O, BaO, NaCl, and CsF.

The compound for the electron transport auxiliary layer (ETAL), which is disposed between the electron transport layer and the emitting layer, is not particularly limited, and may be any compound, as long as it is conventionally used for the electron transport auxiliary layer. For example, the electron transport auxiliary layer may include a pyrimidine derivative, etc.

The organic light emitting diode according to an embodiment of the present disclosure may be a top-emission type or a bottom-emission type.

The organic light emitting diode according to an embodiment of the present disclosure may be applied to a display device.

The organic light emitting diode according to an embodiment of the present disclosure may be applied to transparent display devices, mobile display devices, and flexible display devices, but the present disclosure is not limited thereto.

The organic light emitting diode according to an embodiment of the present disclosure may include a tandem structure including a plurality of emitting stacks between the anode and the cathode.

Hereinafter, a representative example of the synthesis method for the above compounds will be described. However, the methods for synthesizing the compounds of the present disclosure are not limited to the method exemplified below, and the practice of the present disclosure is not limited to the following examples and experimental examples.

### Synthesis Example

A representative synthesis example for Compound 1 (P1) is described below. Compounds represented by Chemical Formula 1 according to the present disclosure may be synthesized in a manner analogous to the reaction of Compound 1 (P1).

In the following reaction scheme, the solvent, catalyst, protecting group, leaving group, reaction temperature, reaction time, and molar equivalents of reactants, etc. are merely representative examples, and any equivalent solvent, catalyst, protecting group, leaving group, reaction temperature, reaction time, or molar equivalents of reactants may also be used.

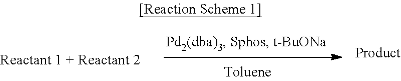

Under a nitrogen atmosphere, reactant 1 of P1 (44 mmol), reactant 2 of P1 (40 mmol), t-BuONa (80 mmol), Pd₂(dba)₃ (0.8 mmol), SPhos (1.6 mmol), and toluene were added to a reaction flask, and the mixture was stirred under reflux. After completion of the reaction, the organic layer was extracted with toluene and water. The extracted solution was dried over MgSO₄ to remove residual moisture, concentrated under reduced pressure, purified by column chromatography, and then recrystallized to obtain the product of P1. The synthesis results for the product of P1 are shown in Table 1 below.

Representative synthesized compounds are shown in Table 1 below, and the specific compounds of the present disclosure and similar compounds can be synthesized through the above synthetic examples.

TABLE 1

| Item | Reactant 1 | Reactant 2 | |
|---|---|---|---|
| P1 | 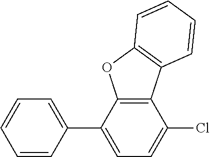 [SMILES: Clc1ccc(-c2ccccc2)c2oc3ccccc3c12] | 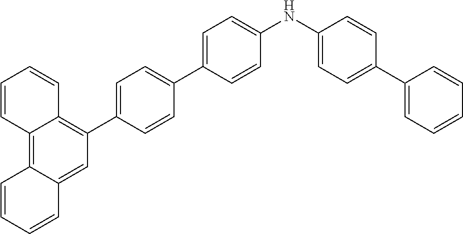 [SMILES: c1ccc(-c2ccc(Nc3ccc(-c4ccc(-c5cc6ccccc6c6ccccc56)cc4)cc3)cc2)cc1] | |
| P2 | 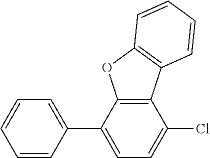 [SMILES: Clc1ccc(-c2ccccc2)c2oc3ccccc3c12] | 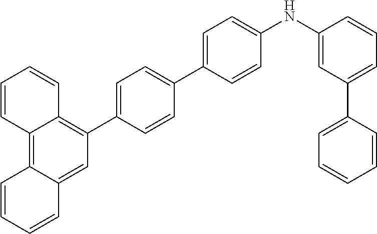 [SMILES: c1ccc(-c2cccc(Nc3ccc(-c4ccc(-c5cc6ccccc6c6ccccc56)cc4)cc3)c2)cc1] | |
| P3 | 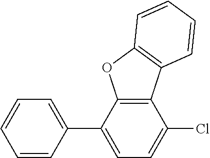 [SMILES: Clc1ccc(-c2ccccc2)c2oc3ccccc3c12] | 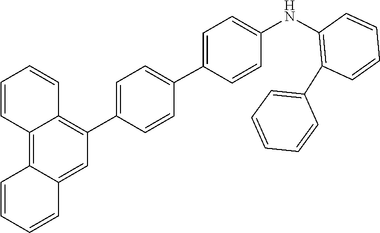 [SMILES: c1ccc(-c2ccccc2Nc2ccc(-c3ccc(-c4cc5ccccc5c5ccccc45)cc3)cc2)cc1] | |
| P4 | 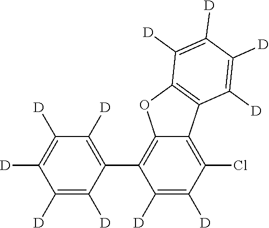 [SMILES: [2H]c1c([2H])c([2H])c(-c2c([2H])c([2H])c(Cl)c3c2oc2c([2H])c([2H])c([2H])c([2H])c23)c([2H])c1[2H]] | 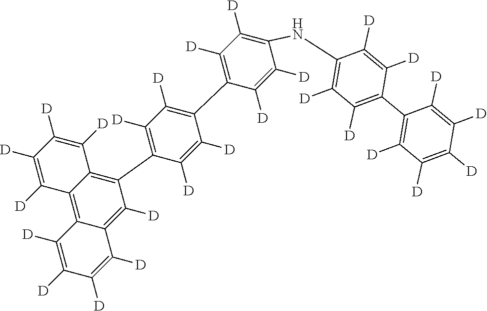 [SMILES: [2H]c1c([2H])c([2H])c(-c2c([2H])c([2H])c(Nc3c([2H])c([2H])c(-c4c([2H])c([2H])c(-c5c([2H])c6c([2H])c([2H])c([2H])c([2H])c6c6c([2H])c([2H])c([2H])c([2H])c56)c([2H])c4[2H])c([2H])c3[2H])c([2H])c2[2H])c([2H])c1[2H]] | |
| P5 | 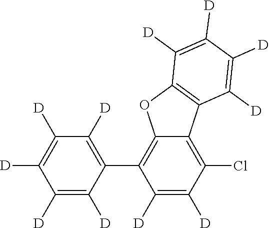 [SMILES: [2H]c1c([2H])c([2H])c(-c2c([2H])c([2H])c(Cl)c3c2oc2c([2H])c([2H])c([2H])c([2H])c23)c([2H])c1[2H]] | 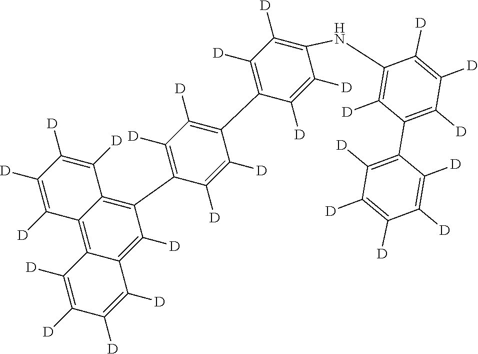 [SMILES: [2H]c1c([2H])c([2H])c(-c2c([2H])c([2H])c([2H])c(Nc3c([2H])c([2H])c(-c4c([2H])c([2H])c(-c5c([2H])c6c([2H])c([2H])c([2H])c([2H])c6c6c([2H])c([2H])c([2H])c([2H])c56)c([2H])c4[2H])c([2H])c3[2H])c2[2H])c([2H])c1[2H]] | |
| P6 | 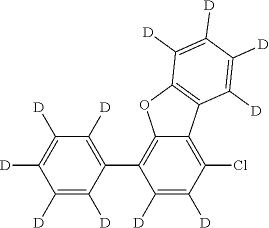 [SMILES: [2H]c1c([2H])c([2H])c(-c2c([2H])c([2H])c(Cl)c3c2oc2c([2H])c([2H])c([2H])c([2H])c23)c([2H])c1[2H]] | 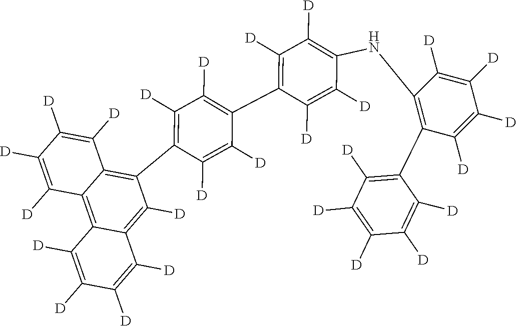 [SMILES: [2H]c1c([2H])c([2H])c(-c2c([2H])c([2H])c([2H])c([2H])c2Nc2c([2H])c([2H])c(-c3c([2H])c([2H])c(-c4c([2H])c5c([2H])c([2H])c([2H])c([2H])c5c5c([2H])c([2H])c([2H])c([2H])c45)c([2H])c3[2H])c([2H])c2[2H])c([2H])c1[2H]] | |
| P7 | 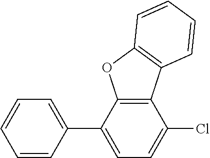 [SMILES: Clc1ccc(-c2ccccc2)c2oc3ccccc3c12] | 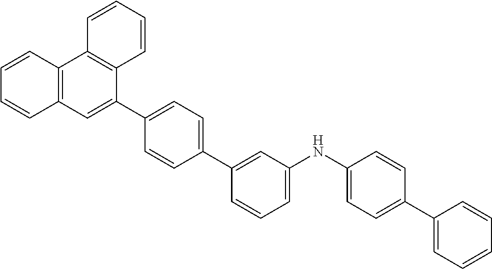 [SMILES: c1ccc(-c2ccc(Nc3cccc(-c4ccc(-c5cc6ccccc6c6ccccc56)cc4)c3)cc2)cc1] | |
| P8 | 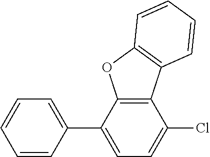 [SMILES: Clc1ccc(-c2ccccc2)c2oc3ccccc3c12] | 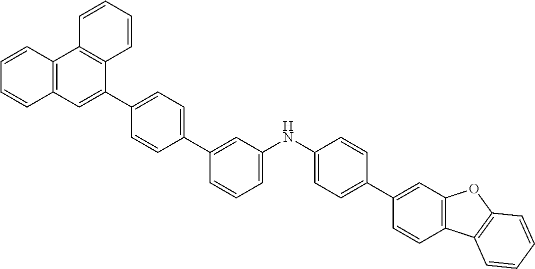 [SMILES: c1cc(Nc2ccc(-c3ccc4c(c3)oc3ccccc34)cc2)cc(-c2ccc(-c3cc4ccccc4c4ccccc34)cc2)c1] | |
| P9 | 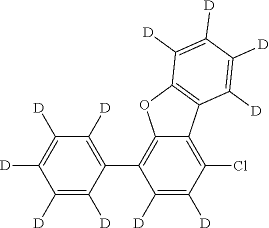 [SMILES: [2H]c1c([2H])c([2H])c(-c2c([2H])c([2H])c(Cl)c3c2oc2c([2H])c([2H])c([2H])c([2H])c23)c([2H])c1[2H]] | 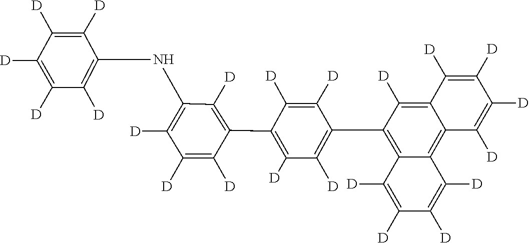 [SMILES: [2H]c1c([2H])c([2H])c(Nc2c([2H])c([2H])c([2H])c(-c3c([2H])c([2H])c(-c4c([2H])c5c([2H])c([2H])c([2H])c([2H])c5c5c([2H])c([2H])c([2H])c([2H])c45)c([2H])c3[2H])c2[2H])c([2H])c1[2H]] | |
| P10 | 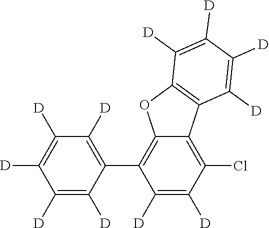 [SMILES: [2H]c1c([2H])c([2H])c(-c2c([2H])c([2H])c(Cl)c3c2oc2c([2H])c([2H])c([2H])c([2H])c23)c([2H])c1[2H]] | 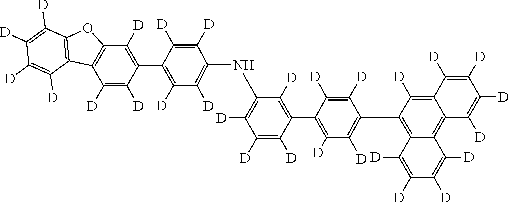 [SMILES: [2H]c1c([2H])c(Nc2c([2H])c([2H])c(-c3c([2H])c([2H])c4c(oc5c([2H])c([2H])c([2H])c([2H])c54)c3[2H])c([2H])c2[2H])c([2H])c(-c2c([2H])c([2H])c(-c3c([2H])c4c([2H])c([2H])c([2H])c([2H])c4c4c([2H])c([2H])c([2H])c([2H])c34)c([2H])c2[2H])c1[2H]] | |
| P11 | 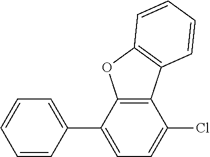 [SMILES: Clc1ccc(-c2ccccc2)c2oc3ccccc3c12] | 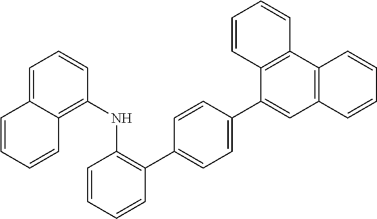 [SMILES: c1ccc(-c2ccc(-c3cc4ccccc4c4ccccc34)cc2)c(Nc2cccc3ccccc23)c1] | |
| P12 | 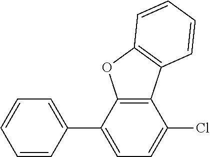 [SMILES: Clc1ccc(-c2ccccc2)c2oc3ccccc3c12] | 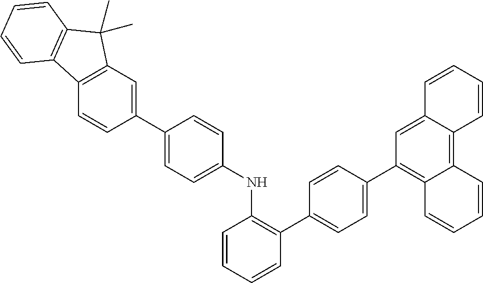 [SMILES: CC1(C)c2ccccc2-c2ccc(-c3ccc(Nc4ccccc4-c4ccc(-c5cc6ccccc6c6ccccc56)cc4)cc3)cc21] | |
| P13 | 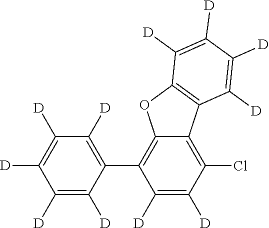 [SMILES: [2H]c1c([2H])c([2H])c(-c2c([2H])c([2H])c(Cl)c3c2oc2c([2H])c([2H])c([2H])c([2H])c23)c([2H])c1[2H]] | 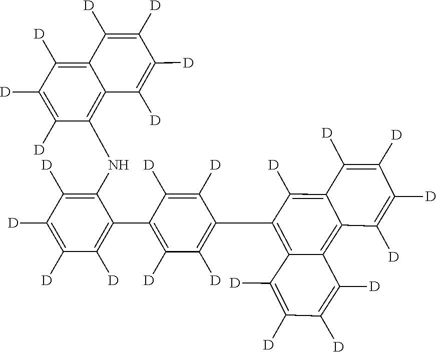 [SMILES: [2H]c1c([2H])c([2H])c(-c2c([2H])c([2H])c(-c3c([2H])c4c([2H])c([2H])c([2H])c([2H])c4c4c([2H])c([2H])c([2H])c([2H])c34)c([2H])c2[2H])c(Nc2c([2H])c([2H])c([2H])c3c([2H])c([2H])c([2H])c([2H])c23)c1[2H]] | |
| P14 | 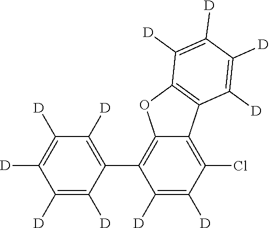 [SMILES: [2H]c1c([2H])c([2H])c(-c2c([2H])c([2H])c(Cl)c3c2oc2c([2H])c([2H])c([2H])c([2H])c23)c([2H])c1[2H]] | 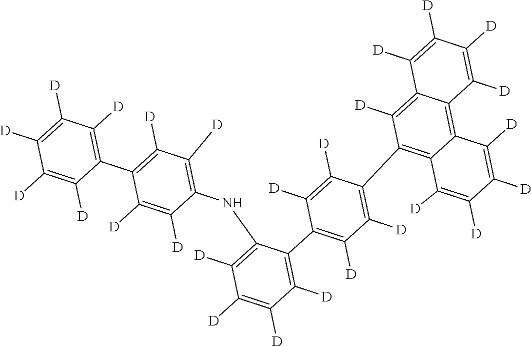 [SMILES: [2H]c1c([2H])c([2H])c(-c2c([2H])c([2H])c(Nc3c([2H])c([2H])c([2H])c([2H])c3-c3c([2H])c([2H])c(-c4c([2H])c5c([2H])c([2H])c([2H])c([2H])c5c5c([2H])c([2H])c([2H])c([2H])c45)c([2H])c3[2H])c([2H])c2[2H])c([2H])c1[2H]] | |
| P15 | 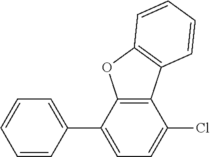 [SMILES: Clc1ccc(-c2ccccc2)c2oc3ccccc3c12] | 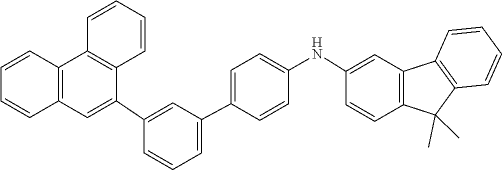 [SMILES: CC1(C)c2ccccc2-c2cc(Nc3ccc(-c4cccc(-c5cc6ccccc6c6ccccc56)c4)cc3)ccc21] | |
| P16 | 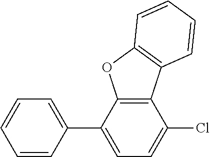 [SMILES: Clc1ccc(-c2ccccc2)c2oc3ccccc3c12] | 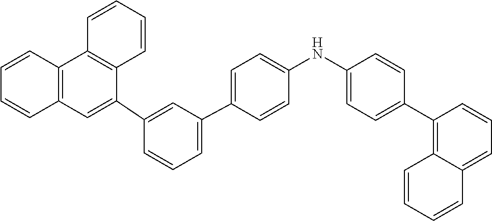 [SMILES: c1cc(-c2ccc(Nc3ccc(-c4cccc5ccccc45)cc3)cc2)cc(-c2cc3ccccc3c3ccccc23)c1] | |
| P17 | 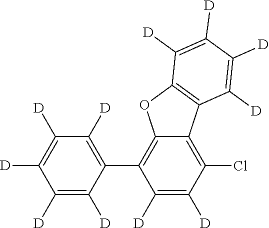 [SMILES: [2H]c1c([2H])c([2H])c(-c2c([2H])c([2H])c(Cl)c3c2oc2c([2H])c([2H])c([2H])c([2H])c23)c([2H])c1[2H]] | 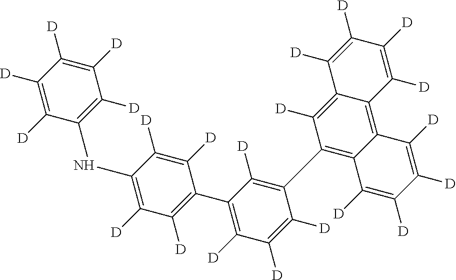 [SMILES: [2H]c1c([2H])c([2H])c(Nc2c([2H])c([2H])c(-c3c([2H])c([2H])c([2H])c(-c4c([2H])c5c([2H])c([2H])c([2H])c([2H])c5c5c([2H])c([2H])c([2H])c([2H])c45)c3[2H])c([2H])c2[2H])c([2H])c1[2H]] | |
| P18 | 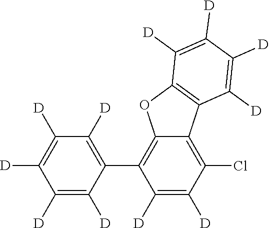 [SMILES: [2H]c1c([2H])c([2H])c(-c2c([2H])c([2H])c(Cl)c3c2oc2c([2H])c([2H])c([2H])c([2H])c23)c([2H])c1[2H]] | 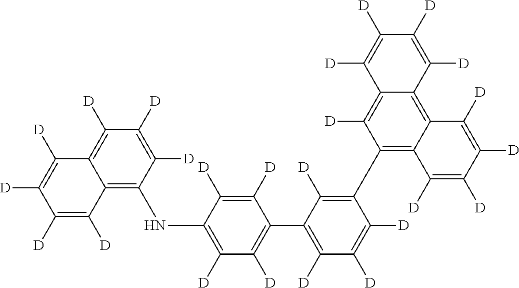 [SMILES: [2H]c1c([2H])c(-c2c([2H])c([2H])c(Nc3c([2H])c([2H])c([2H])c4c([2H])c([2H])c([2H])c([2H])c34)c([2H])c2[2H])c([2H])c(-c2c([2H])c3c([2H])c([2H])c([2H])c([2H])c3c3c([2H])c([2H])c([2H])c([2H])c23)c1[2H]] | |
| P19 | 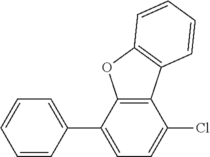 [SMILES: Clc1ccc(-c2ccccc2)c2oc3ccccc3c12] | 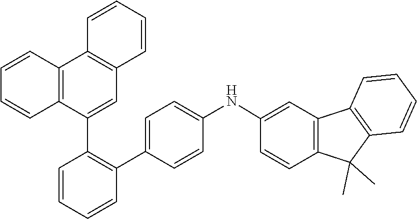 [SMILES: CC1(C)c2ccccc2-c2cc(Nc3ccc(-c4ccccc4-c4cc5ccccc5c5ccccc45)cc3)ccc21] | |
| P20 | 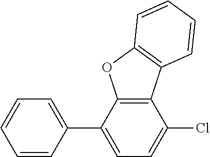 [SMILES: Clc1ccc(-c2ccccc2)c2oc3ccccc3c12] | 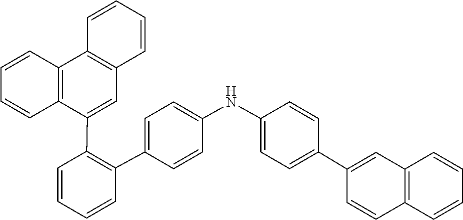 [SMILES: c1ccc(-c2cc3ccccc3c3ccccc23)c(-c2ccc(Nc3ccc(-c4ccc5ccccc5c4)cc3)cc2)c1] | |
| P21 | 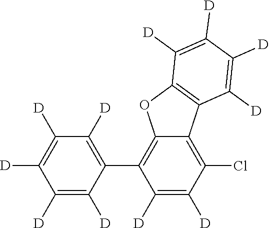 [SMILES: [2H]c1c([2H])c([2H])c(-c2c([2H])c([2H])c(Cl)c3c2oc2c([2H])c([2H])c([2H])c([2H])c23)c([2H])c1[2H]] | 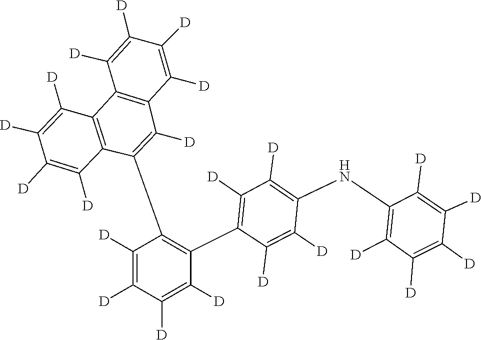 [SMILES: [2H]c1c([2H])c([2H])c(Nc2c([2H])c([2H])c(-c3c([2H])c([2H])c([2H])c([2H])c3-c3c([2H])c4c([2H])c([2H])c([2H])c([2H])c4c4c([2H])c([2H])c([2H])c([2H])c34)c([2H])c2[2H])c([2H])c1[2H]] | |
| P22 | 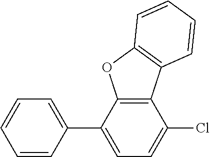 [SMILES: Clc1ccc(-c2ccccc2)c2oc3ccccc3c12] | 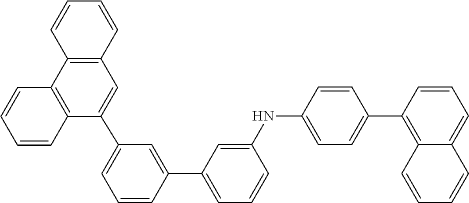 [SMILES: c1cc(Nc2ccc(-c3cccc4ccccc34)cc2)cc(-c2cccc(-c3cc4ccccc4c4ccccc34)c2)c1] | |
| P23 | 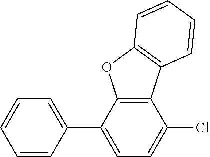 [SMILES: Clc1ccc(-c2ccccc2)c2oc3ccccc3c12] | 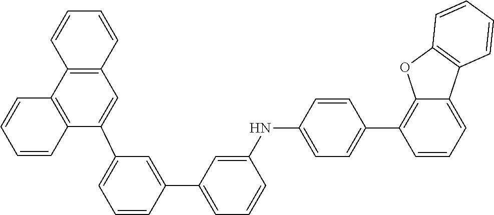 [SMILES: c1cc(Nc2ccc(-c3cccc4c3oc3ccccc34)cc2)cc(-c2cccc(-c3cc4ccccc4c4ccccc34)c2)c1] | |
| P24 | 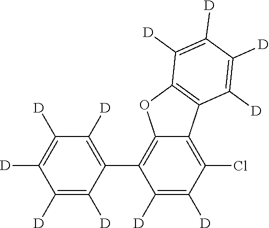 [SMILES: [2H]c1c([2H])c([2H])c(-c2c([2H])c([2H])c(Cl)c3c2oc2c([2H])c([2H])c([2H])c([2H])c23)c([2H])c1[2H]] | 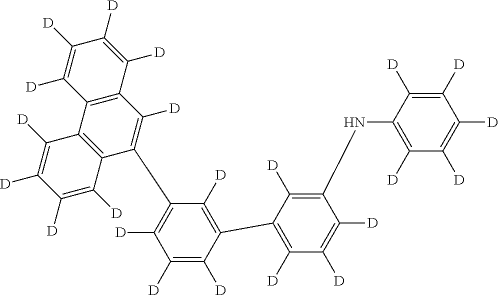 [SMILES: [2H]c1c([2H])c([2H])c(Nc2c([2H])c([2H])c([2H])c(-c3c([2H])c([2H])c([2H])c(-c4c([2H])c5c([2H])c([2H])c([2H])c([2H])c5c5c([2H])c([2H])c([2H])c([2H])c45)c3[2H])c2[2H])c([2H])c1[2H]] | |
| P25 | 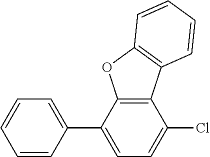 [SMILES: Clc1ccc(-c2ccccc2)c2oc3ccccc3c12] | 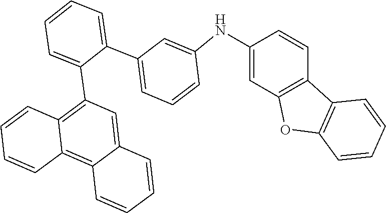 [SMILES: c1cc(Nc2ccc3c(c2)oc2ccccc23)cc(-c2ccccc2-c2cc3ccccc3c3ccccc23)c1] | |
| P26 | 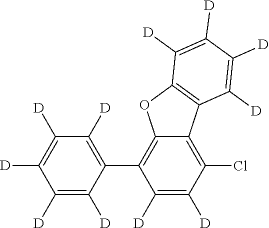 [SMILES: [2H]c1c([2H])c([2H])c(-c2c([2H])c([2H])c(Cl)c3c2oc2c([2H])c([2H])c([2H])c([2H])c23)c([2H])c1[2H]] | 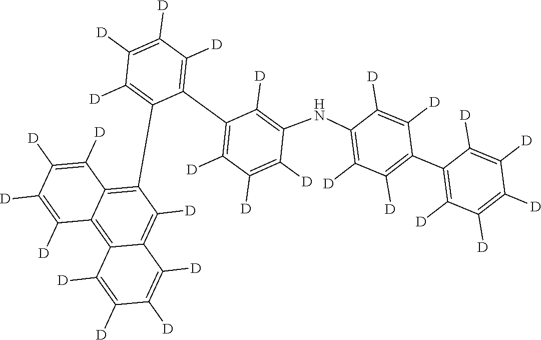 [SMILES: [2H]c1c([2H])c([2H])c(-c2c([2H])c([2H])c(Nc3c([2H])c([2H])c([2H])c(-c4c([2H])c([2H])c([2H])c([2H])c4-c4c([2H])c5c([2H])c([2H])c([2H])c([2H])c5c5c([2H])c([2H])c([2H])c([2H])c45)c3[2H])c([2H])c2[2H])c([2H])c1[2H]] | |
| P27 | 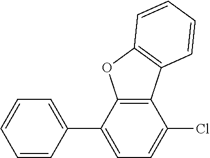 [SMILES: Clc1ccc(-c2ccccc2)c2oc3ccccc3c12] | 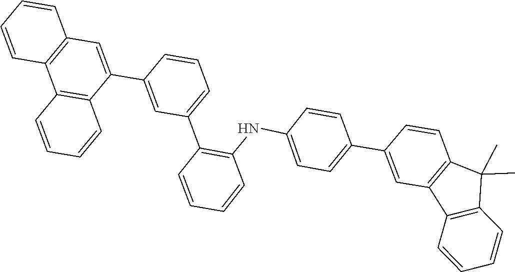 [SMILES: CC1(C)c2ccccc2-c2cc(-c3ccc(Nc4ccccc4-c4cccc(-c5cc6ccccc6c6ccccc56)c4)cc3)ccc21] | |
| P28 | 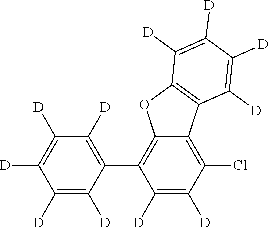 [SMILES: [2H]c1c([2H])c([2H])c(-c2c([2H])c([2H])c(Cl)c3c2oc2c([2H])c([2H])c([2H])c([2H])c23)c([2H])c1[2H]] | 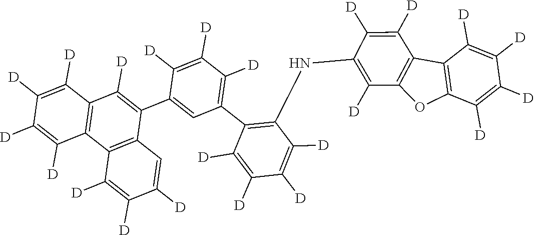 [SMILES: [2H]c1cc2c(-c3cc(-c4c([2H])c([2H])c([2H])c([2H])c4Nc4c([2H])c([2H])c5c(oc6c([2H])c([2H])c([2H])c([2H])c65)c4[2H])c([2H])c([2H])c3[2H])c([2H])c3c([2H])c([2H])c([2H])c([2H])c3c2c([2H])c1[2H]] | |
| P29 | 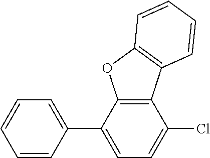 [SMILES: Clc1ccc(-c2ccccc2)c2oc3ccccc3c12] | 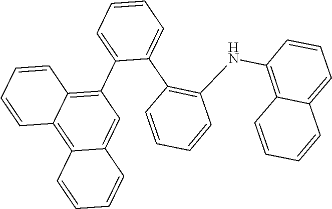 [SMILES: c1ccc(-c2ccccc2-c2cc3ccccc3c3ccccc23)c(Nc2cccc3ccccc23)c1] | |
| P30 | 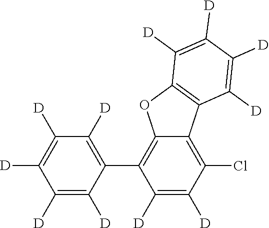 [SMILES: [2H]c1c([2H])c([2H])c(-c2c([2H])c([2H])c(Cl)c3c2oc2c([2H])c([2H])c([2H])c([2H])c23)c([2H])c1[2H]] | 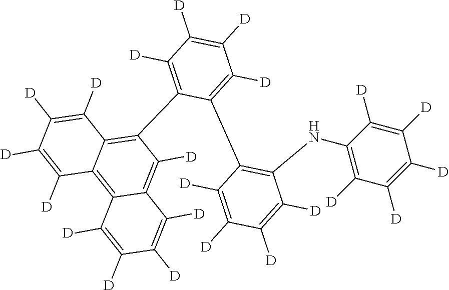 [SMILES: [2H]c1c([2H])c([2H])c(Nc2c([2H])c([2H])c([2H])c([2H])c2-c2c([2H])c([2H])c([2H])c([2H])c2-c2c([2H])c3c([2H])c([2H])c([2H])c([2H])c3c3c([2H])c([2H])c([2H])c([2H])c23)c([2H])c1[2H]] | |
| P31 | 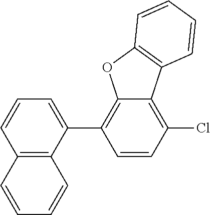 [SMILES: Clc1ccc(-c2cccc3ccccc23)c2oc3ccccc3c12] | 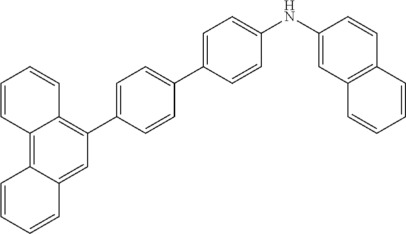 [SMILES: c1ccc2cc(Nc3ccc(-c4ccc(-c5cc6ccccc6c6ccccc56)cc4)cc3)ccc2c1] | |
| P32 | 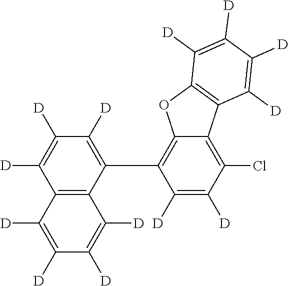 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | 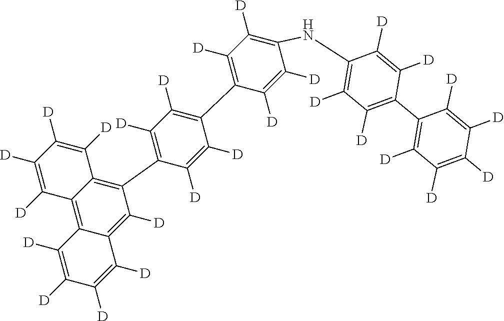 [SMILES: [2H]c1c([2H])c([2H])c(-c2c([2H])c([2H])c(Nc3c([2H])c([2H])c(-c4c([2H])c([2H])c(-c5c([2H])c6c([2H])c([2H])c([2H])c([2H])c6c6c([2H])c([2H])c([2H])c([2H])c56)c([2H])c4[2H])c([2H])c3[2H])c([2H])c2[2H])c([2H])c1[2H]] | |
| P33 | 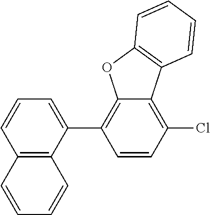 [SMILES: Clc1ccc(-c2cccc3ccccc23)c2oc3ccccc3c12] | 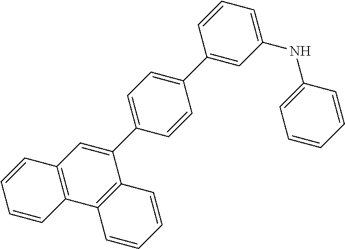 [SMILES: c1ccc(Nc2cccc(-c3ccc(-c4cc5ccccc5c5ccccc45)cc3)c2)cc1] | |
| P34 | 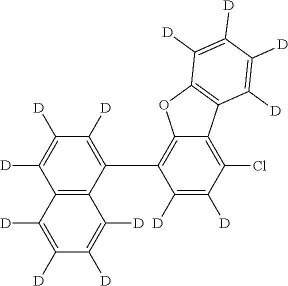 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | 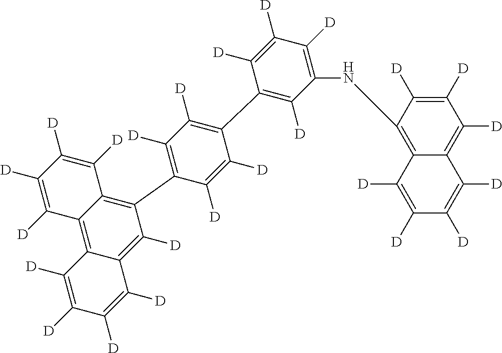 [SMILES: [2H]c1c([2H])c(Nc2c([2H])c([2H])c([2H])c3c([2H])c([2H])c([2H])c([2H])c23)c([2H])c(-c2c([2H])c([2H])c(-c3c([2H])c4c([2H])c([2H])c([2H])c([2H])c4c4c([2H])c([2H])c([2H])c([2H])c34)c([2H])c2[2H])c1[2H]] | |
| P35 | 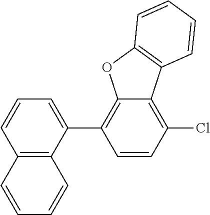 [SMILES: Clc1ccc(-c2cccc3ccccc23)c2oc3ccccc3c12] | 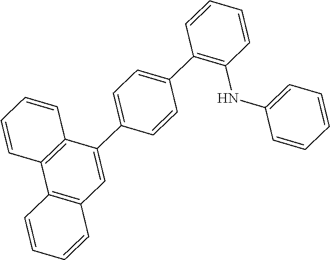 [SMILES: c1ccc(Nc2ccccc2-c2ccc(-c3cc4ccccc4c4ccccc34)cc2)cc1] | |
| P36 | 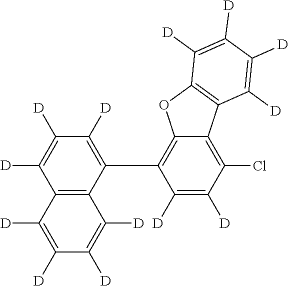 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | 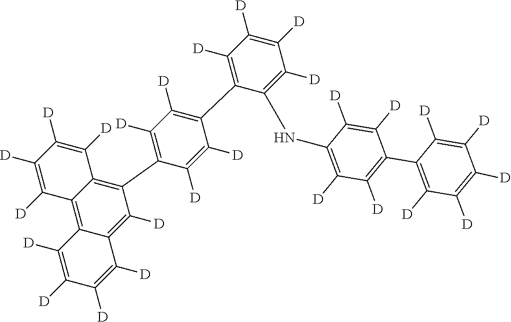 [SMILES: [2H]c1c([2H])c([2H])c(-c2c([2H])c([2H])c(Nc3c([2H])c([2H])c([2H])c([2H])c3-c3c([2H])c([2H])c(-c4c([2H])c5c([2H])c([2H])c([2H])c([2H])c5c5c([2H])c([2H])c([2H])c([2H])c45)c([2H])c3[2H])c([2H])c2[2H])c([2H])c1[2H]] | |
| P37 | 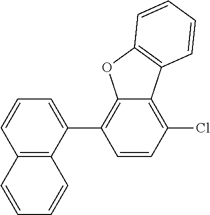 [SMILES: Clc1ccc(-c2cccc3ccccc23)c2oc3ccccc3c12] | 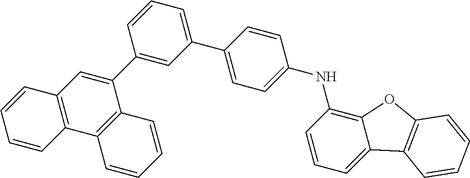 [SMILES: c1cc(-c2ccc(Nc3cccc4c3oc3ccccc34)cc2)cc(-c2cc3ccccc3c3ccccc23)c1] | |
| P38 | 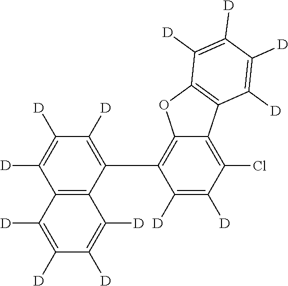 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | 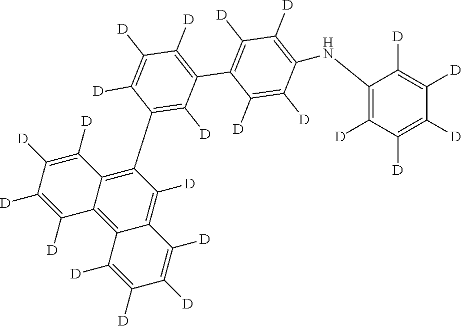 [SMILES: [2H]c1c([2H])c([2H])c(Nc2c([2H])c([2H])c(-c3c([2H])c([2H])c([2H])c(-c4c([2H])c5c([2H])c([2H])c([2H])c([2H])c5c5c([2H])c([2H])c([2H])c([2H])c45)c3[2H])c([2H])c2[2H])c([2H])c1[2H]] | |
| P39 | 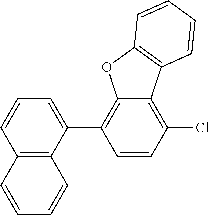 [SMILES: Clc1ccc(-c2cccc3ccccc23)c2oc3ccccc3c12] | 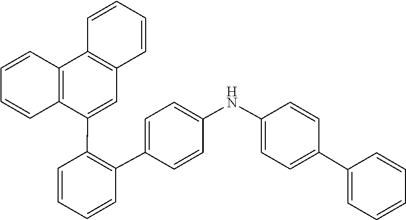 [SMILES: c1ccc(-c2ccc(Nc3ccc(-c4ccccc4-c4cc5ccccc5c5ccccc45)cc3)cc2)cc1] | |
| P40 | 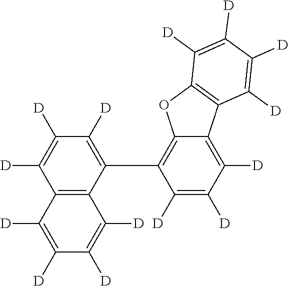 [SMILES: [2H]c1c([2H])c([2H])c2c(oc3c(-c4c([2H])c([2H])c([2H])c5c([2H])c([2H])c([2H])c([2H])c45)c([2H])c([2H])c([2H])c32)c1[2H]] | 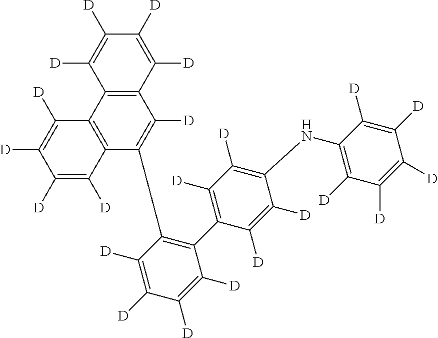 [SMILES: [2H]c1c([2H])c([2H])c(Nc2c([2H])c([2H])c(-c3c([2H])c([2H])c([2H])c([2H])c3-c3c([2H])c4c([2H])c([2H])c([2H])c([2H])c4c4c([2H])c([2H])c([2H])c([2H])c34)c([2H])c2[2H])c([2H])c1[2H]] | |
| P41 | 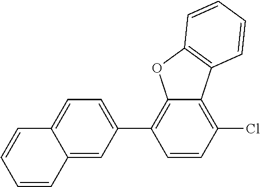 [SMILES: Clc1ccc(-c2ccc3ccccc3c2)c2oc3ccccc3c12] | 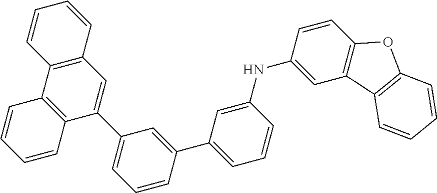 [SMILES: c1cc(Nc2ccc3oc4ccccc4c3c2)cc(-c2cccc(-c3cc4ccccc4c4ccccc34)c2)c1] | |
| P42 | 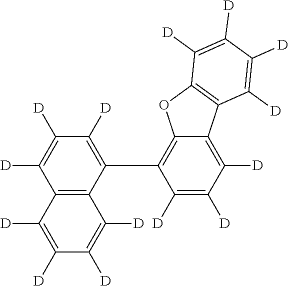 [SMILES: [2H]c1c([2H])c([2H])c2c(oc3c(-c4c([2H])c([2H])c([2H])c5c([2H])c([2H])c([2H])c([2H])c45)c([2H])c([2H])c([2H])c32)c1[2H]] | 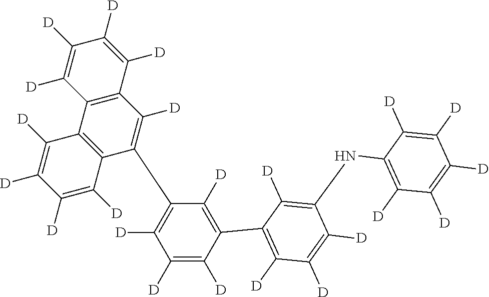 [SMILES: [2H]c1c([2H])c([2H])c(Nc2c([2H])c([2H])c([2H])c(-c3c([2H])c([2H])c([2H])c(-c4c([2H])c5c([2H])c([2H])c([2H])c([2H])c5c5c([2H])c([2H])c([2H])c([2H])c45)c3[2H])c2[2H])c([2H])c1[2H]] | |
| P43 | 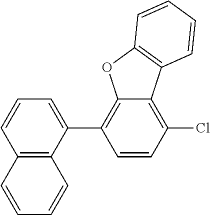 [SMILES: Clc1ccc(-c2cccc3ccccc23)c2oc3ccccc3c12] | 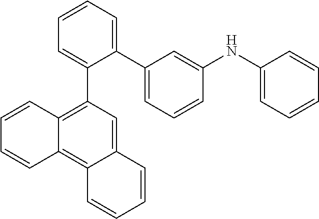 [SMILES: c1ccc(Nc2cccc(-c3ccccc3-c3cc4ccccc4c4ccccc34)c2)cc1] | |
| P44 | 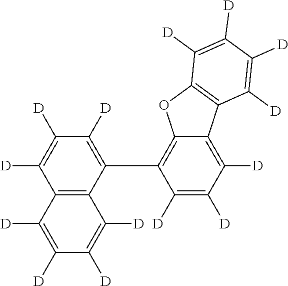 [SMILES: [2H]c1c([2H])c([2H])c2c(oc3c(-c4c([2H])c([2H])c([2H])c5c([2H])c([2H])c([2H])c([2H])c45)c([2H])c([2H])c([2H])c32)c1[2H]] | 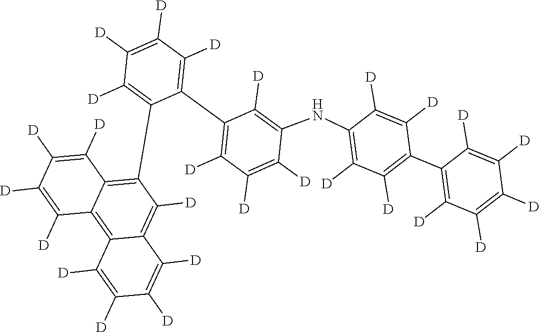 [SMILES: [2H]c1c([2H])c([2H])c(-c2c([2H])c([2H])c(Nc3c([2H])c([2H])c([2H])c(-c4c([2H])c([2H])c([2H])c([2H])c4-c4c([2H])c5c([2H])c([2H])c([2H])c([2H])c5c5c([2H])c([2H])c([2H])c([2H])c45)c3[2H])c([2H])c2[2H])c([2H])c1[2H]] | |
| P45 | 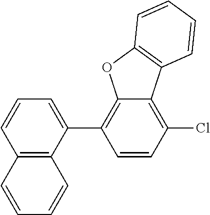 [SMILES: Clc1ccc(-c2cccc3ccccc23)c2oc3ccccc3c12] | 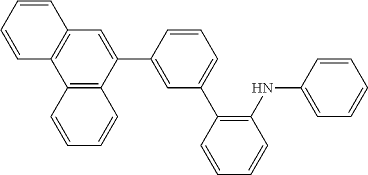 [SMILES: c1ccc(Nc2ccccc2-c2cccc(-c3cc4ccccc4c4ccccc34)c2)cc1] | |
| P46 | 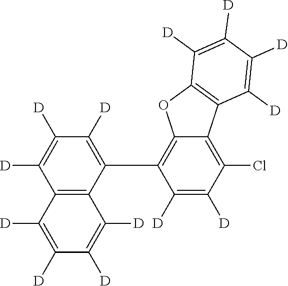 [SMILES: [2H]c1c([2H])c([2H])c2c(oc3c(-c4c([2H])c([2H])c([2H])c5c([2H])c([2H])c([2H])c([2H])c45)c([2H])c([2H])c(Cl)c32)c1[2H]] | 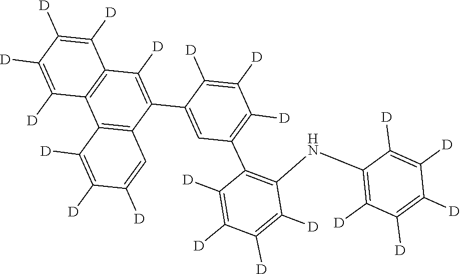 [SMILES: [2H]c1cc2c(-c3cc(-c4c([2H])c([2H])c([2H])c([2H])c4Nc4c([2H])c([2H])c([2H])c([2H])c4[2H])c([2H])c([2H])c3[2H])c([2H])c3c([2H])c([2H])c([2H])c([2H])c3c2c([2H])c1[2H]] | |
| P47 | 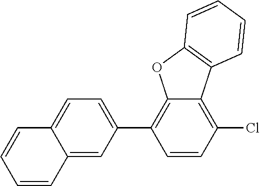 [SMILES: Clc1ccc(-c2ccc3ccccc3c2)c2oc3ccccc3c12] | 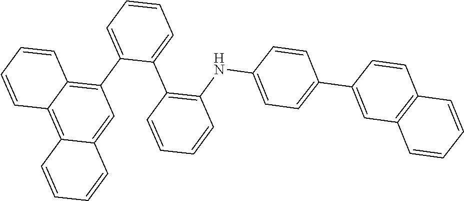 [SMILES: c1ccc(-c2ccccc2-c2cc3ccccc3c3ccccc23)c(Nc2ccc(-c3ccc4ccccc4c3)cc2)c1] | |
| P48 | 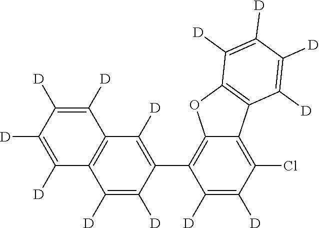 [SMILES: [2H]c1c([2H])c([2H])c2c(oc3c(-c4c([2H])c([2H])c5c([2H])c([2H])c([2H])c([2H])c5c4[2H])c([2H])c([2H])c(Cl)c32)c1[2H]] | 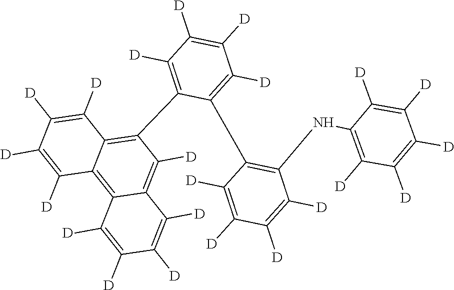 [SMILES: [2H]c1c([2H])c([2H])c(Nc2c([2H])c([2H])c([2H])c([2H])c2-c2c([2H])c([2H])c([2H])c([2H])c2-c2c([2H])c3c([2H])c([2H])c([2H])c([2H])c3c3c([2H])c([2H])c([2H])c([2H])c23)c([2H])c1[2H]] | |
| P49 | 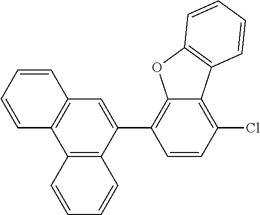 [SMILES: Clc1ccc(-c2cc3ccccc3c3ccccc23)c2oc3ccccc3c12] | 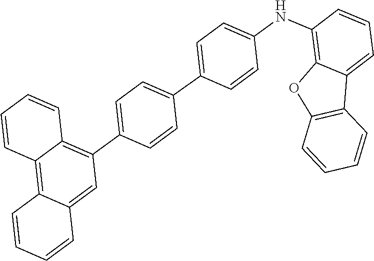 [SMILES: c1ccc2c(c1)cc(-c1ccc(-c3ccc(Nc4cccc5c4oc4ccccc45)cc3)cc1)c1ccccc12] | |
| P50 | 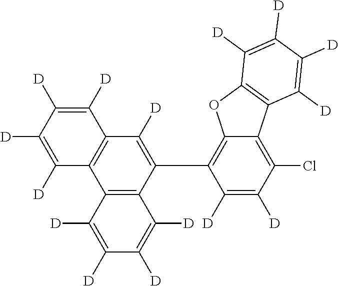 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 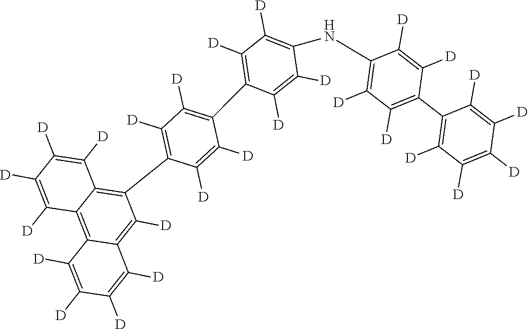 [SMILES: [2H]c1c([2H])c([2H])c(-c2c([2H])c([2H])c(Nc3c([2H])c([2H])c(-c4c([2H])c([2H])c(-c5c([2H])c6c([2H])c([2H])c([2H])c([2H])c6c6c([2H])c([2H])c([2H])c([2H])c56)c([2H])c4[2H])c([2H])c3[2H])c([2H])c2[2H])c([2H])c1[2H]] | |
| P51 | 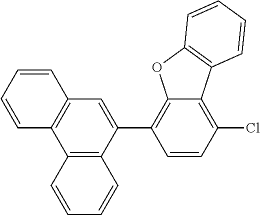 [SMILES: Clc1ccc(-c2cc3ccccc3c3ccccc23)c2oc3ccccc3c12] | 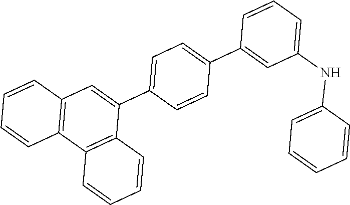 [SMILES: c1ccc(Nc2cccc(-c3ccc(-c4cc5ccccc5c5ccccc45)cc3)c2)cc1] | |
| P52 | 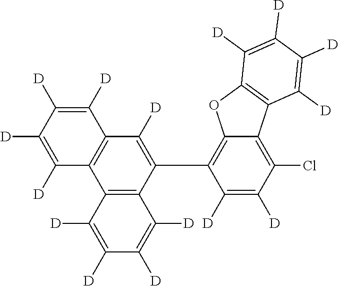 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 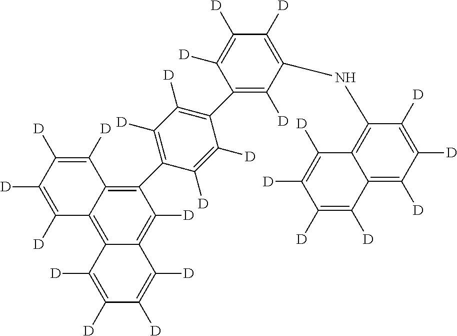 [SMILES: [2H]c1c([2H])c(Nc2c([2H])c([2H])c([2H])c3c([2H])c([2H])c([2H])c([2H])c23)c([2H])c(-c2c([2H])c([2H])c(-c3c([2H])c4c([2H])c([2H])c([2H])c([2H])c4c4c([2H])c([2H])c([2H])c([2H])c34)c([2H])c2[2H])c1[2H]] | |
| P53 | 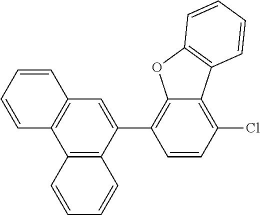 [SMILES: Clc1ccc(-c2cc3ccccc3c3ccccc23)c2oc3ccccc3c12] | 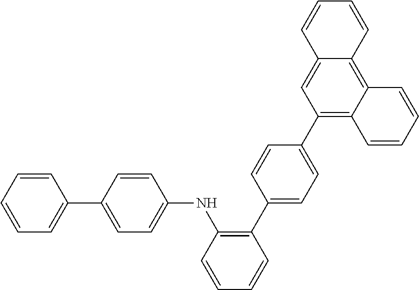 [SMILES: c1ccc(-c2ccc(Nc3ccccc3-c3ccc(-c4cc5ccccc5c5ccccc45)cc3)cc2)cc1] | |
| P54 | 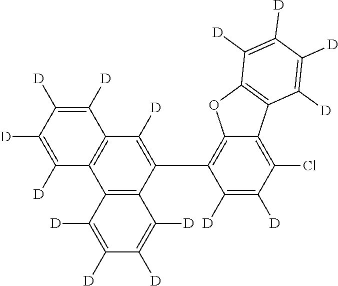 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 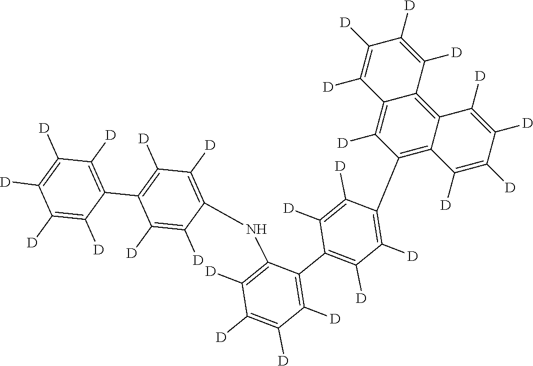 [SMILES: [2H]c1c([2H])c([2H])c(-c2c([2H])c([2H])c(Nc3c([2H])c([2H])c([2H])c([2H])c3-c3c([2H])c([2H])c(-c4c([2H])c5c([2H])c([2H])c([2H])c([2H])c5c5c([2H])c([2H])c([2H])c([2H])c45)c([2H])c3[2H])c([2H])c2[2H])c([2H])c1[2H]] | |
| P55 | 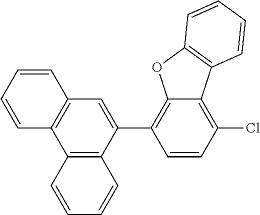 [SMILES: Clc1ccc(-c2cc3ccccc3c3ccccc23)c2oc3ccccc3c12] | 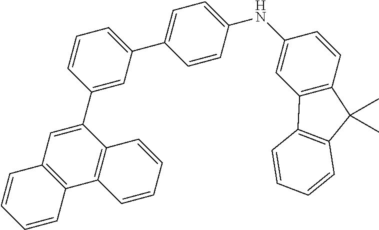 [SMILES: CC1(C)c2ccccc2-c2cc(Nc3ccc(-c4cccc(-c5cc6ccccc6c6ccccc56)c4)cc3)ccc21] | |
| P56 | 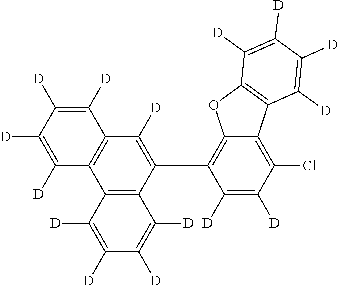 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 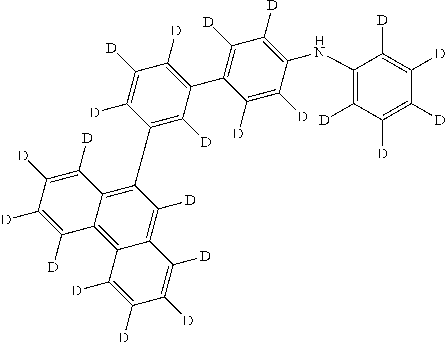 [SMILES: [2H]c1c([2H])c([2H])c(Nc2c([2H])c([2H])c(-c3c([2H])c([2H])c([2H])c(-c4c([2H])c5c([2H])c([2H])c([2H])c([2H])c5c5c([2H])c([2H])c([2H])c([2H])c45)c3[2H])c([2H])c2[2H])c([2H])c1[2H]] | |
| P57 | 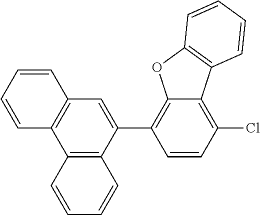 [SMILES: Clc1ccc(-c2cc3ccccc3c3ccccc23)c2oc3ccccc3c12] | 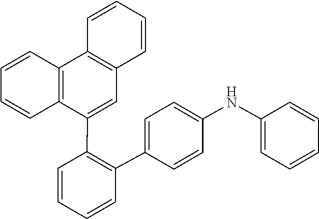 [SMILES: c1ccc(Nc2ccc(-c3ccccc3-c3cc4ccccc4c4ccccc34)cc2)cc1] | |
| P58 | 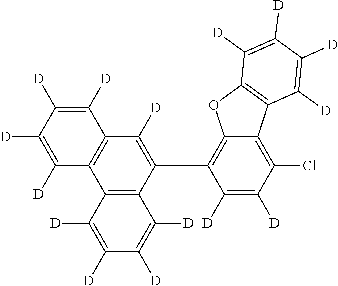 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 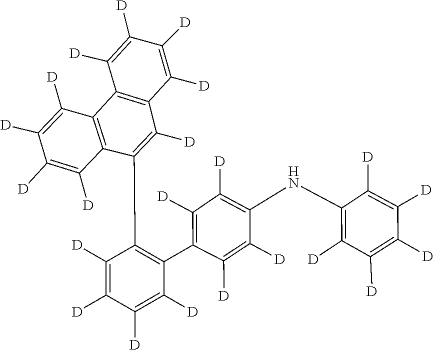 [SMILES: [2H]c1c([2H])c([2H])c(Nc2c([2H])c([2H])c(-c3c([2H])c([2H])c([2H])c([2H])c3-c3c([2H])c4c([2H])c([2H])c([2H])c([2H])c4c4c([2H])c([2H])c([2H])c([2H])c34)c([2H])c2[2H])c([2H])c1[2H]] | |
| P59 | 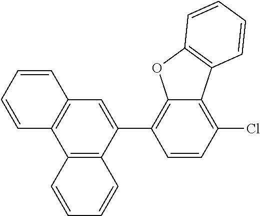 [SMILES: Clc1ccc(-c2cc3ccccc3c3ccccc23)c2oc3ccccc3c12] | 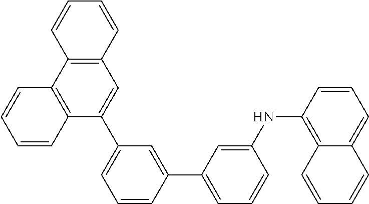 [SMILES: c1cc(Nc2cccc3ccccc23)cc(-c2cccc(-c3cc4ccccc4c4ccccc34)c2)c1] | |
| P60 | 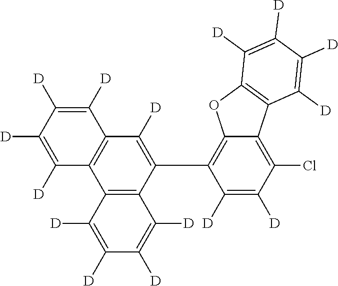 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 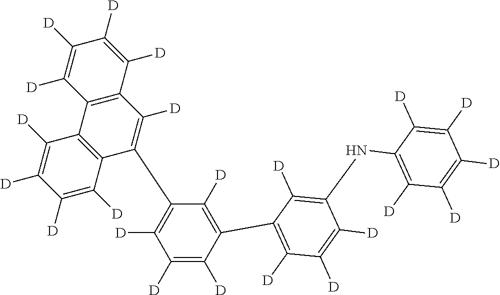 [SMILES: [2H]c1c([2H])c([2H])c(Nc2c([2H])c([2H])c([2H])c(-c3c([2H])c([2H])c([2H])c(-c4c([2H])c5c([2H])c([2H])c([2H])c([2H])c5c5c([2H])c([2H])c([2H])c([2H])c45)c3[2H])c2[2H])c([2H])c1[2H]] | |
| P61 | 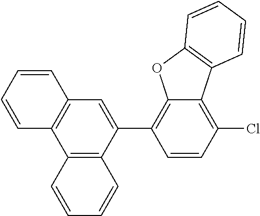 [SMILES: Clc1ccc(-c2cc3ccccc3c3ccccc23)c2oc3ccccc3c12] | 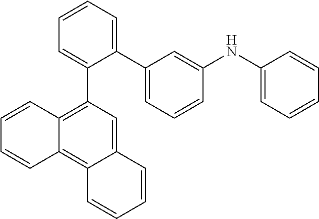 [SMILES: c1ccc(Nc2cccc(-c3ccccc3-c3cc4ccccc4c4ccccc34)c2)cc1] | |
| P62 | 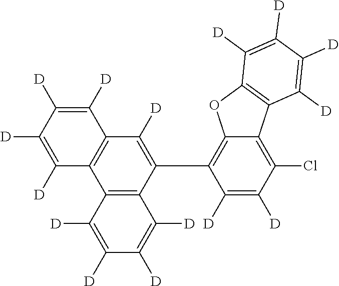 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 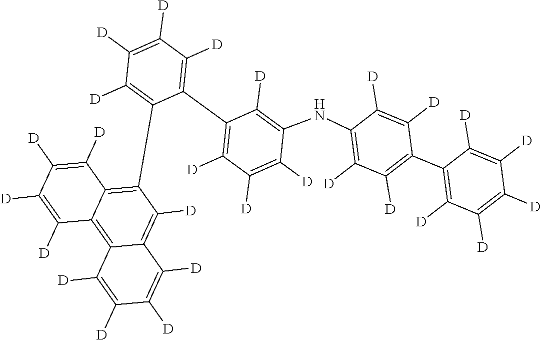 [SMILES: [2H]c1c([2H])c([2H])c(-c2c([2H])c([2H])c(Nc3c([2H])c([2H])c([2H])c(-c4c([2H])c([2H])c([2H])c([2H])c4-c4c([2H])c5c([2H])c([2H])c([2H])c([2H])c5c5c([2H])c([2H])c([2H])c([2H])c45)c3[2H])c([2H])c2[2H])c([2H])c1[2H]] | |
| P63 | 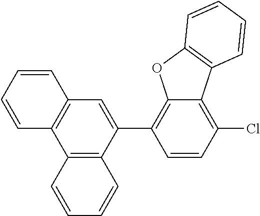 [SMILES: Clc1ccc(-c2cc3ccccc3c3ccccc23)c2oc3ccccc3c12] | 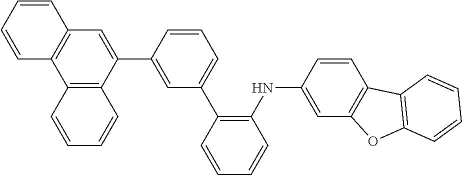 [SMILES: c1cc(-c2ccccc2Nc2ccc3c(c2)oc2ccccc23)cc(-c2cc3ccccc3c3ccccc23)c1] | |
| P64 | 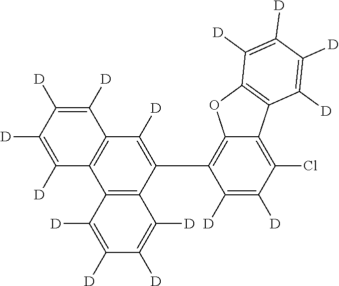 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 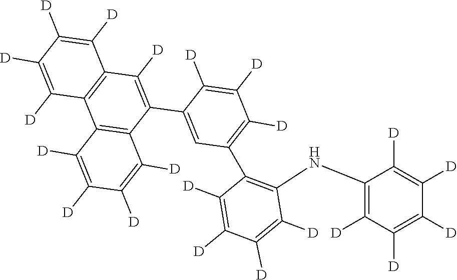 [SMILES: [2H]c1c(-c2c([2H])c([2H])c([2H])c([2H])c2Nc2c([2H])c([2H])c([2H])c([2H])c2[2H])cc(-c2c([2H])c3c([2H])c([2H])c([2H])c([2H])c3c3c([2H])c([2H])c([2H])c([2H])c23)c([2H])c1[2H]] | |
| P65 | 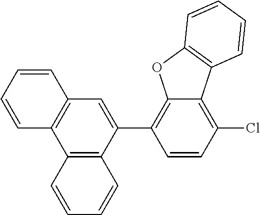 [SMILES: Clc1ccc(-c2cc3ccccc3c3ccccc23)c2oc3ccccc3c12] | 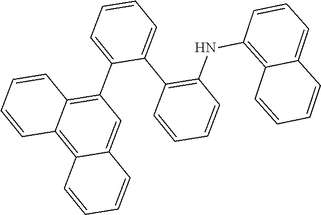 [SMILES: c1ccc(-c2ccccc2-c2cc3ccccc3c3ccccc23)c(Nc2cccc3ccccc23)c1] | |
| P66 | 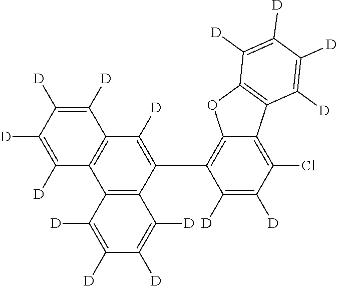 [SMILES: [2H]c1c([2H])c([2H])c2c(oc3c(-c4c([2H])c5c([2H])c([2H])c([2H])c([2H])c5c5c([2H])c([2H])c([2H])c([2H])c45)c([2H])c([2H])c(Cl)c32)c1[2H]] | 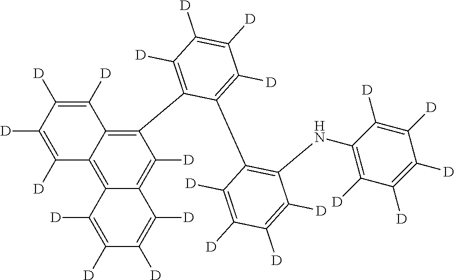 [SMILES: [2H]c1c([2H])c([2H])c(Nc2c([2H])c([2H])c([2H])c([2H])c2-c2c([2H])c([2H])c([2H])c([2H])c2-c2c([2H])c3c([2H])c([2H])c([2H])c([2H])c3c3c([2H])c([2H])c([2H])c([2H])c23)c([2H])c1[2H]] | |
| Item | Product | Yield | [M + H]⁺ |
| P1 | 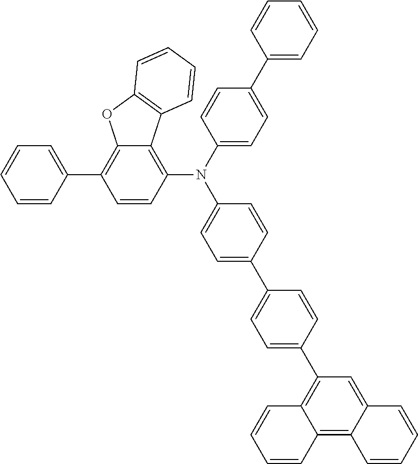 [SMILES: c1ccc(-c2ccc(N(c3ccc(-c4ccc(-c5cc6ccccc6c6ccccc56)cc4)cc3)c3ccc(-c4ccccc4)c4oc5ccccc5c34)cc2)cc1] | 22.5 g (76%) | 739.29 |
| | Compound 17 | | |
| P2 | 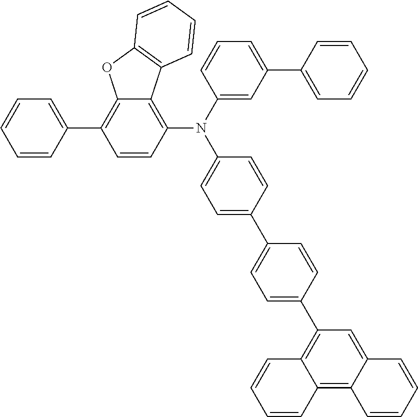 [SMILES: c1ccc(-c2cccc(N(c3ccc(-c4ccc(-c5cc6ccccc6c6ccccc56)cc4)cc3)c3ccc(-c4ccccc4)c4oc5ccccc5c34)c2)cc1] | 21.3 g (72%) | 739.29 |
| | Compound 21 | | |
| P3 | 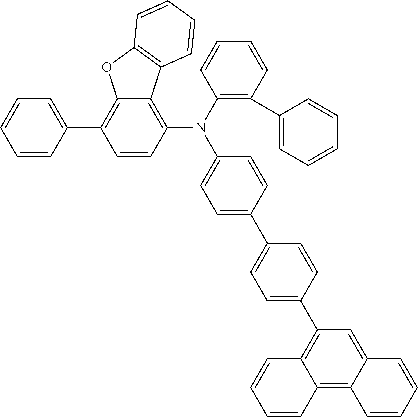 [SMILES: c1ccc(-c2ccccc2N(c2ccc(-c3ccc(-c4cc5ccccc5c5ccccc45)cc3)cc2)c2ccc(-c3ccccc3)c3oc4ccccc4c23)cc1] | 20.7 g (70%) | 739.29 |
| | Compound 25 | | |
| P4 | 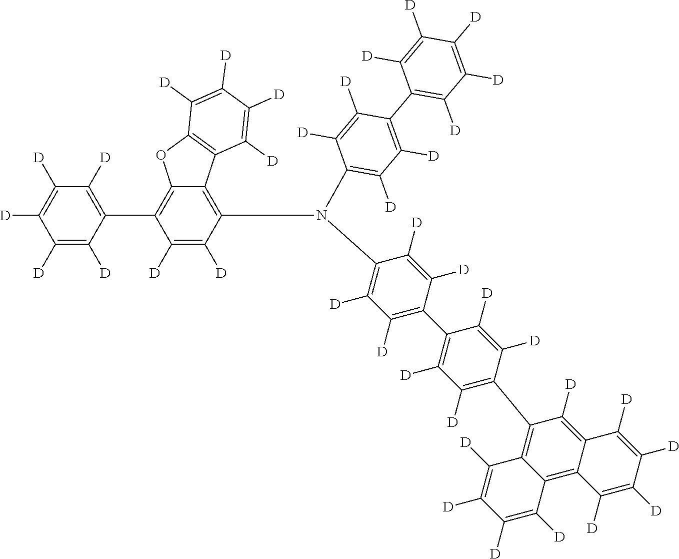 [SMILES: [2H]c1c([2H])c([2H])c(-c2c([2H])c([2H])c(N(c3c([2H])c([2H])c(-c4c([2H])c([2H])c(-c5c([2H])c6c([2H])c([2H])c([2H])c([2H])c6c6c([2H])c([2H])c([2H])c([2H])c56)c([2H])c4[2H])c([2H])c3[2H])c3c([2H])c([2H])c(-c4c([2H])c([2H])c([2H])c([2H])c4[2H])c4oc5c([2H])c([2H])c([2H])c([2H])c5c34)c([2H])c2[2H])c([2H])c1[2H]] | 22.7 g (73%) | 776.52 |
| | Compound 63 | | |
| P5 | 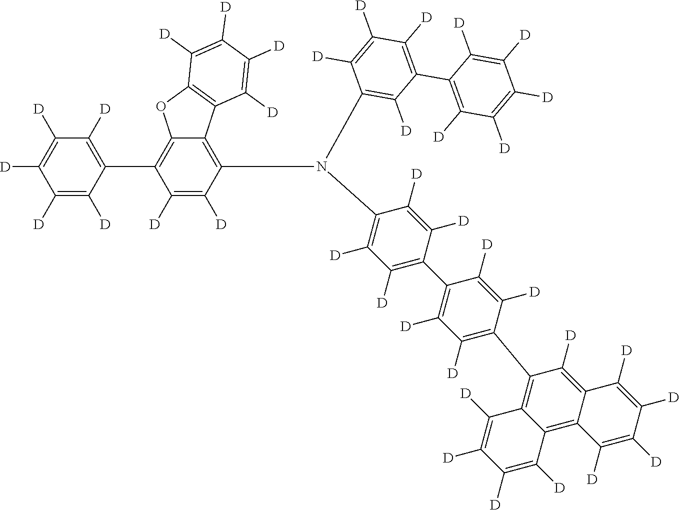 [SMILES: [2H]c1c([2H])c([2H])c(-c2c([2H])c([2H])c([2H])c(N(c3c([2H])c([2H])c(-c4c([2H])c([2H])c(-c5c([2H])c6c([2H])c([2H])c([2H])c([2H])c6c6c([2H])c([2H])c([2H])c([2H])c56)c([2H])c4[2H])c([2H])c3[2H])c3c([2H])c([2H])c(-c4c([2H])c([2H])c([2H])c([2H])c4[2H])c4oc5c([2H])c([2H])c([2H])c([2H])c5c34)c2[2H])c([2H])c1[2H]] | 22.4 g (72%) | 776.52 |
| | Compound 66 | | |
| P6 | 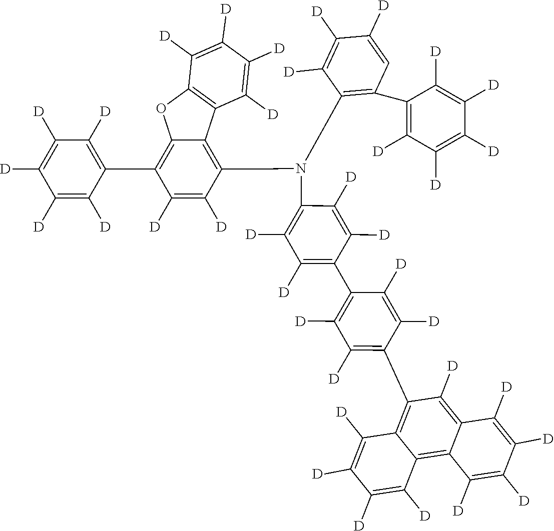 [SMILES: [2H]c1cc(-c2c([2H])c([2H])c([2H])c([2H])c2[2H])c(N(c2c([2H])c([2H])c(-c3c([2H])c([2H])c(-c4c([2H])c5c([2H])c([2H])c([2H])c([2H])c5c5c([2H])c([2H])c([2H])c([2H])c45)c([2H])c3[2H])c([2H])c2[2H])c2c([2H])c([2H])c(-c3c([2H])c([2H])c([2H])c([2H])c3[2H])c3oc4c([2H])c([2H])c([2H])c([2H])c4c23)c([2H])c1[2H]] | 22.1 g (71%) | 776.52 |
| | Compound 69 | | |
| P7 | 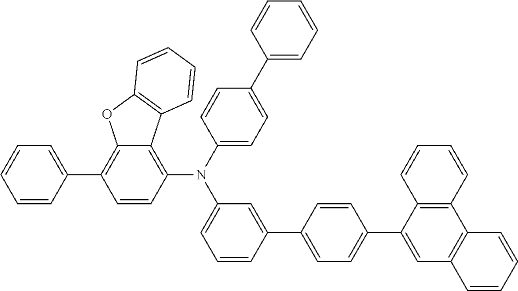 [SMILES: c1ccc(-c2ccc(N(c3cccc(-c4ccc(-c5cc6ccccc6c6ccccc56)cc4)c3)c3ccc(-c4ccccc4)c4oc5ccccc5c34)cc2)cc1] | 19.8 g (67%) | 739.29 |
| | Compound 86 | | |
| P8 | 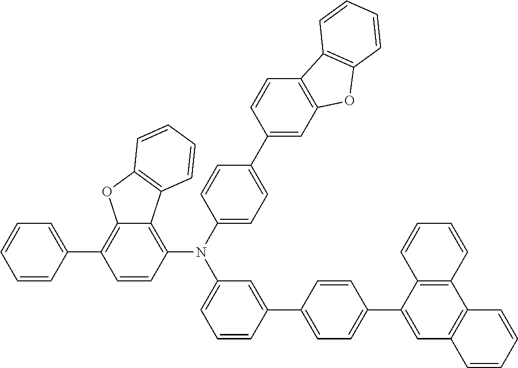 [SMILES: c1ccc(-c2ccc(N(c3ccc(-c4ccc5c(c4)oc4ccccc45)cc3)c3cccc(-c4ccc(-c5cc6ccccc6c6ccccc56)cc4)c3)c3c2oc2ccccc23)cc1] | 23.2 g (70%) | 829.30 |
| | Compound 99 | | |
| P9 | 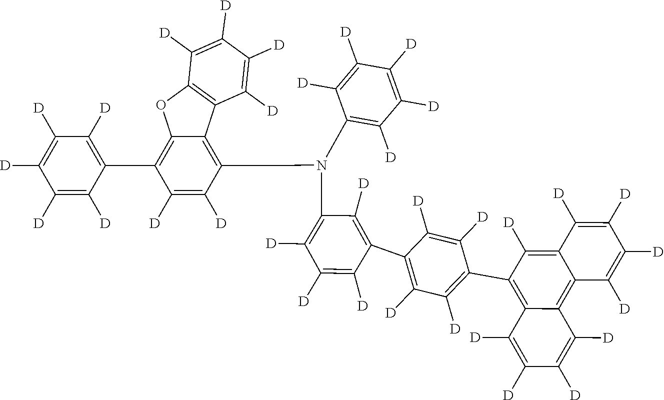 [SMILES: [2H]c1c([2H])c([2H])c(-c2c([2H])c([2H])c(N(c3c([2H])c([2H])c([2H])c([2H])c3[2H])c3c([2H])c([2H])c([2H])c(-c4c([2H])c([2H])c(-c5c([2H])c6c([2H])c([2H])c([2H])c([2H])c6c6c([2H])c([2H])c([2H])c([2H])c56)c([2H])c4[2H])c3[2H])c3c2oc2c([2H])c([2H])c([2H])c([2H])c23)c([2H])c1[2H]] | 20.6 g (74%) | 696.46 |
| | Compound 128 | | |
| P10 | 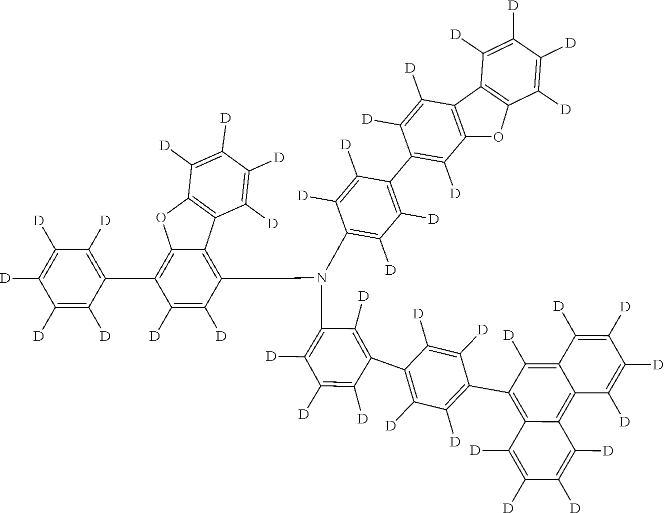 [SMILES: [2H]c1c([2H])c([2H])c(-c2c([2H])c([2H])c(N(c3c([2H])c([2H])c(-c4c([2H])c([2H])c5c(oc6c([2H])c([2H])c([2H])c([2H])c65)c4[2H])c([2H])c3[2H])c3c([2H])c([2H])c([2H])c(-c4c([2H])c([2H])c(-c5c([2H])c6c([2H])c([2H])c([2H])c([2H])c6c6c([2H])c([2H])c([2H])c([2H])c56)c([2H])c4[2H])c3[2H])c3c2oc2c([2H])c([2H])c([2H])c([2H])c23)c([2H])c1[2H]] | 24.7 g (71%) | 868.54 |
| | Compound 131 | | |
| P11 | 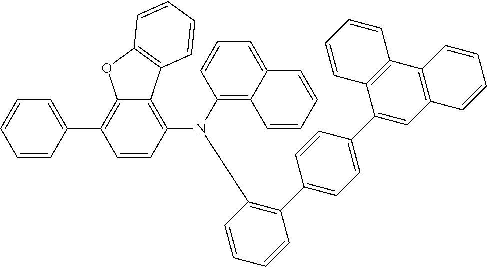 [SMILES: c1ccc(-c2ccc(N(c3ccccc3-c3ccc(-c4cc5ccccc5c5ccccc45)cc3)c3cccc4ccccc34)c3c2oc2ccccc23)cc1] | 17.7 g (62%) | 713.27 |
| | Compound 133 | | |
| P12 | 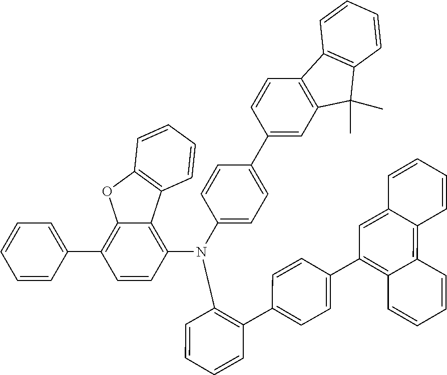 [SMILES: CC1(C)c2ccccc2-c2ccc(-c3ccc(N(c4ccccc4-c4ccc(-c5cc6ccccc6c6ccccc56)cc4)c4ccc(-c5ccccc5)c5oc6ccccc6c45)cc3)cc21] | 21.6 g (63%) | 855.35 |
| | Compound 153 | | |
| P13 | 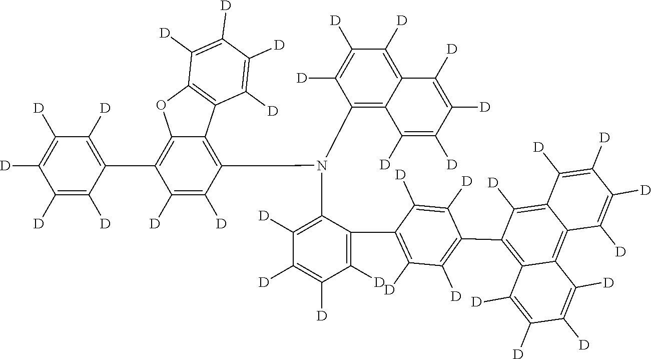 [SMILES: [2H]c1c([2H])c([2H])c(-c2c([2H])c([2H])c(N(c3c([2H])c([2H])c([2H])c([2H])c3-c3c([2H])c([2H])c(-c4c([2H])c5c([2H])c([2H])c([2H])c([2H])c5c5c([2H])c([2H])c([2H])c([2H])c45)c([2H])c3[2H])c3c([2H])c([2H])c([2H])c4c([2H])c([2H])c([2H])c([2H])c34)c3c2oc2c([2H])c([2H])c([2H])c([2H])c23)c([2H])c1[2H]] | 18.3 g (61%) | 748.49 |
| | Compound 178 | | |
| P14 | 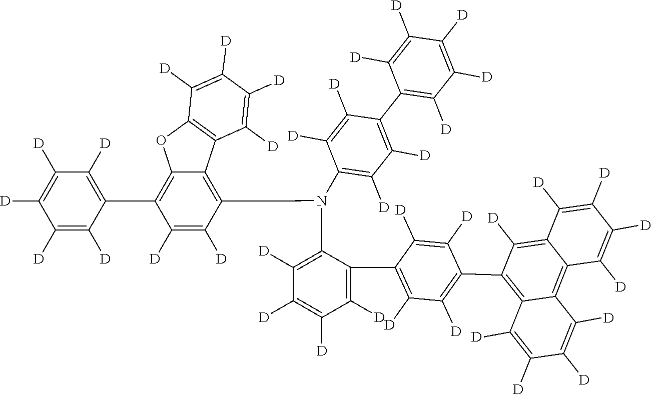 [SMILES: [2H]c1c([2H])c([2H])c(-c2c([2H])c([2H])c(N(c3c([2H])c([2H])c([2H])c([2H])c3-c3c([2H])c([2H])c(-c4c([2H])c5c([2H])c([2H])c([2H])c([2H])c5c5c([2H])c([2H])c([2H])c([2H])c45)c([2H])c3[2H])c3c([2H])c([2H])c(-c4c([2H])c([2H])c([2H])c([2H])c4[2H])c4oc5c([2H])c([2H])c([2H])c([2H])c5c34)c([2H])c2[2H])c([2H])c1[2H]] | 19.3 g (62%) | 776.52 |
| | Compound 181 | | |
| P15 | 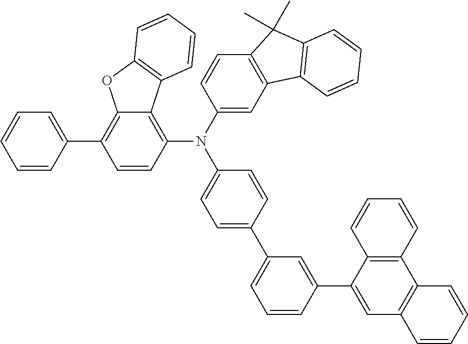 [SMILES: CC1(C)c2ccccc2-c2cc(N(c3ccc(-c4cccc(-c5cc6ccccc6c6ccccc56)c4)cc3)c3ccc(-c4ccccc4)c4oc5ccccc5c34)ccc21] | 23.1 g (74%) | 779.32 |
| | Compound 196 | | |
| P16 | 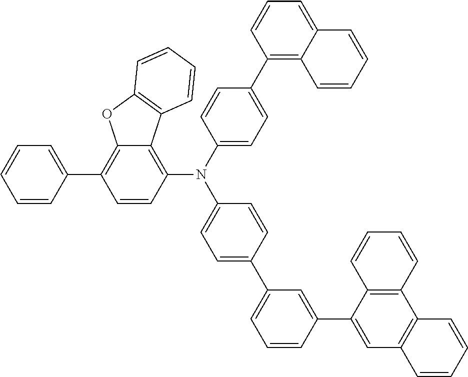 [SMILES: c1ccc(-c2ccc(N(c3ccc(-c4cccc(-c5cc6ccccc6c6ccccc56)c4)cc3)c3ccc(-c4cccc5ccccc45)cc3)c3c2oc2ccccc23)cc1] | 22.1 g (70%) | 789.30 |
| | Compound 199 | | |
| P17 | 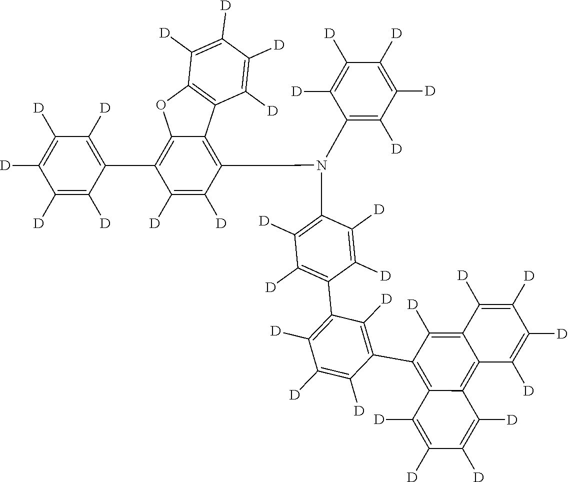 [SMILES: [2H]c1c([2H])c([2H])c(-c2c([2H])c([2H])c(N(c3c([2H])c([2H])c([2H])c([2H])c3[2H])c3c([2H])c([2H])c(-c4c([2H])c([2H])c([2H])c(-c5c([2H])c6c([2H])c([2H])c([2H])c([2H])c6c6c([2H])c([2H])c([2H])c([2H])c56)c4[2H])c([2H])c3[2H])c3c2oc2c([2H])c([2H])c([2H])c([2H])c23)c([2H])c1[2H]] | 20.1 g (72%) | 696.46 |
| | Compound 244 | | |
| P18 | 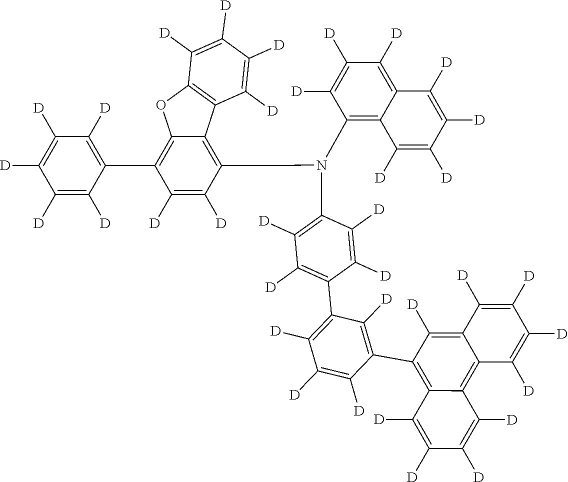 [SMILES: [2H]c1c([2H])c([2H])c(-c2c([2H])c([2H])c(N(c3c([2H])c([2H])c(-c4c([2H])c([2H])c([2H])c(-c5c([2H])c6c([2H])c([2H])c([2H])c([2H])c6c6c([2H])c([2H])c([2H])c([2H])c56)c4[2H])c([2H])c3[2H])c3c([2H])c([2H])c([2H])c4c([2H])c([2H])c([2H])c([2H])c34)c3c2oc2c([2H])c([2H])c([2H])c([2H])c23)c([2H])c1[2H]] | 21.3 g (71%) | 748.49 |
| | Compound 247 | | |
| P19 | 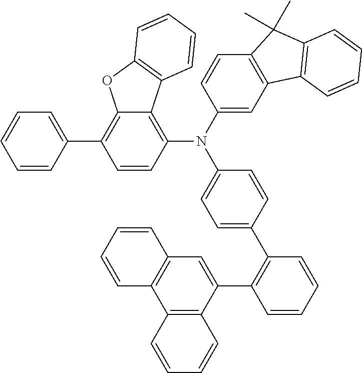 [SMILES: CC1(C)c2ccccc2-c2cc(N(c3ccc(-c4ccccc4-c4cc5ccccc5c5ccccc45)cc3)c3ccc(-c4ccccc4)c4oc5ccccc5c34)ccc21] | 21.2 g (68%) | 779.32 |
| | Compound 1025 | | |
| P20 | 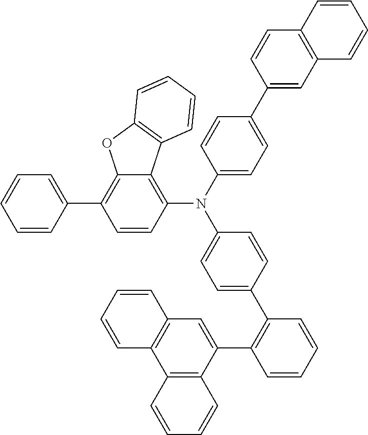 [SMILES: c1ccc(-c2ccc(N(c3ccc(-c4ccc5ccccc5c4)cc3)c3ccc(-c4ccccc4-c4cc5ccccc5c5ccccc45)cc3)c3c2oc2ccccc23)cc1] | 21.8 g (69%) | 789.30 |
| | Compound 258 | | |
| P21 | 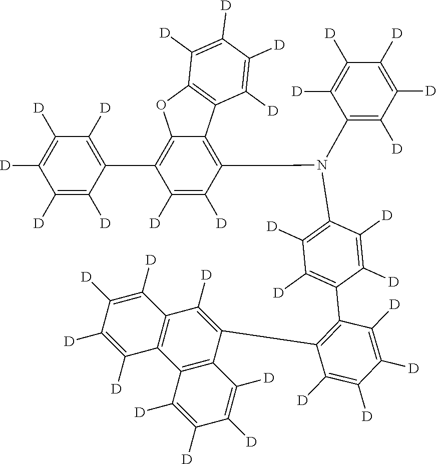 [SMILES: [2H]c1c([2H])c([2H])c(-c2c([2H])c([2H])c(N(c3c([2H])c([2H])c([2H])c([2H])c3[2H])c3c([2H])c([2H])c(-c4c([2H])c([2H])c([2H])c([2H])c4-c4c([2H])c5c([2H])c([2H])c([2H])c([2H])c5c5c([2H])c([2H])c([2H])c([2H])c45)c([2H])c3[2H])c3c2oc2c([2H])c([2H])c([2H])c([2H])c23)c([2H])c1[2H]] | 19.8 g (71%) | 696.46 |
| | Compound 286 | | |
| P22 | 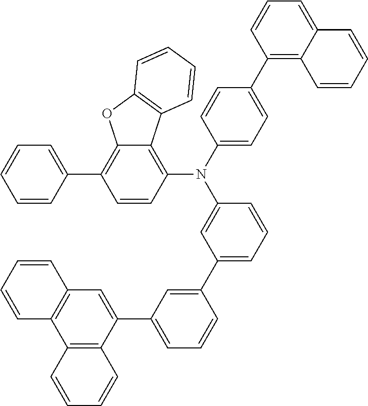 [SMILES: c1ccc(-c2ccc(N(c3ccc(-c4cccc5ccccc45)cc3)c3cccc(-c4cccc(-c5cc6ccccc6c6ccccc56)c4)c3)c3c2oc2ccccc23)cc1] | 21.2 g (67%) | 789.30 |
| | Compound 296 | | |
| P23 | 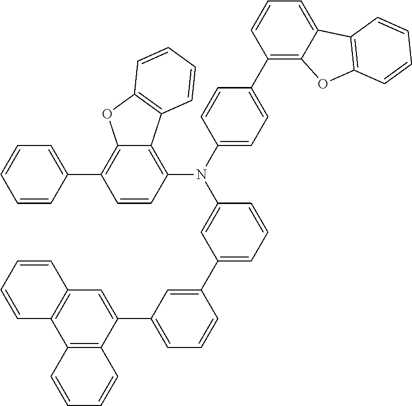 [SMILES: c1ccc(-c2ccc(N(c3ccc(-c4cccc5c4oc4ccccc45)cc3)c3cccc(-c4cccc(-c5cc6ccccc6c6ccccc56)c4)c3)c3c2oc2ccccc23)cc1] | 21.9 g (66%) | 829.30 |
| | Compound 307 | | |
| P24 | 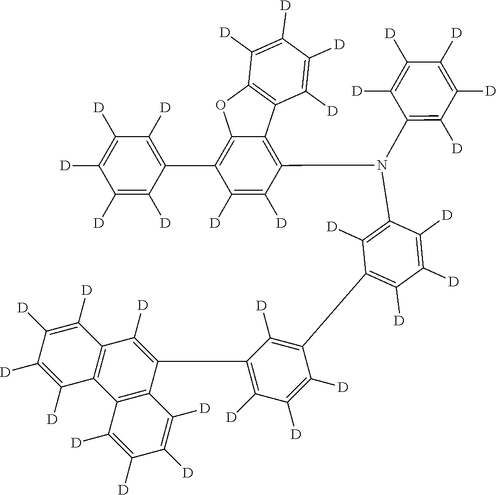 [SMILES: [2H]c1c([2H])c([2H])c(-c2c([2H])c([2H])c(N(c3c([2H])c([2H])c([2H])c([2H])c3[2H])c3c([2H])c([2H])c([2H])c(-c4c([2H])c([2H])c([2H])c(-c5c([2H])c6c([2H])c([2H])c([2H])c([2H])c6c6c([2H])c([2H])c([2H])c([2H])c56)c4[2H])c3[2H])c3c2oc2c([2H])c([2H])c([2H])c([2H])c23)c([2H])c1[2H]] | 19.5 g (70%) | 696.46 |
| | Compound 325 | | |
| P25 | 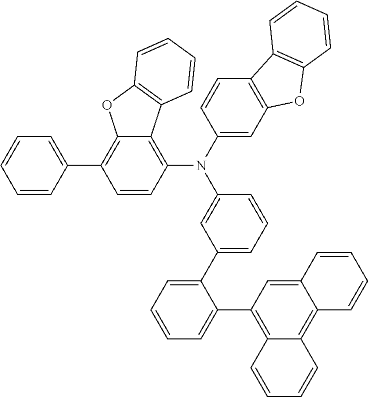 [SMILES: c1ccc(-c2ccc(N(c3cccc(-c4ccccc4-c4cc5ccccc5c5ccccc45)c3)c3ccc4c(c3)oc3ccccc34)c3c2oc2ccccc23)cc1] | 19.0 g (63%) | 753.27 |
| | Compound 1026 | | |
| P26 | 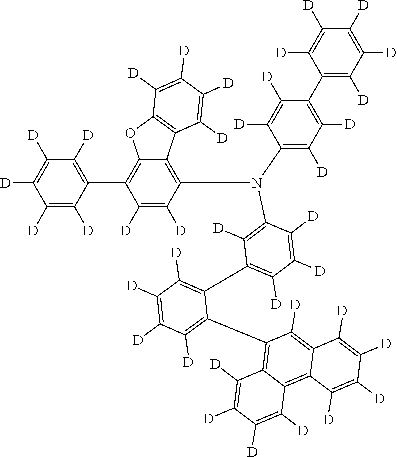 [SMILES: [2H]c1c([2H])c([2H])c(-c2c([2H])c([2H])c(N(c3c([2H])c([2H])c([2H])c(-c4c([2H])c([2H])c([2H])c([2H])c4-c4c([2H])c5c([2H])c([2H])c([2H])c([2H])c5c5c([2H])c([2H])c([2H])c([2H])c45)c3[2H])c3c([2H])c([2H])c(-c4c([2H])c([2H])c([2H])c([2H])c4[2H])c4oc5c([2H])c([2H])c([2H])c([2H])c5c34)c([2H])c2[2H])c([2H])c1[2H]] | 20.5 g (66%) | 776.52 |
| | Compound 364 | | |
| P27 | 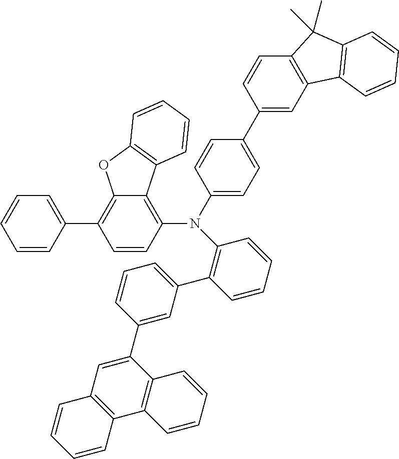 [SMILES: CC1(C)c2ccccc2-c2cc(-c3ccc(N(c4ccccc4-c4cccc(-c5cc6ccccc6c6ccccc56)c4)c4ccc(-c5ccccc5)c5oc6ccccc6c45)cc3)ccc21] | 22.9 g (67%) | 855.35 |
| | Compound 1027 | | |
| P28 | 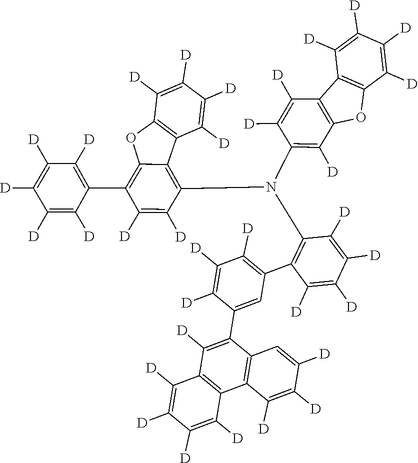 [SMILES: [2H]c1cc2c(-c3cc(-c4c([2H])c([2H])c([2H])c([2H])c4N(c4c([2H])c([2H])c5c(oc6c([2H])c([2H])c([2H])c([2H])c65)c4[2H])c4c([2H])c([2H])c(-c5c([2H])c([2H])c([2H])c([2H])c5[2H])c5oc6c([2H])c([2H])c([2H])c([2H])c6c45)c([2H])c([2H])c3[2H])c([2H])c3c([2H])c([2H])c([2H])c([2H])c3c2c([2H])c1[2H]] | 21.4 g (68%) | 786.47 |
| | Compound 395 | | |
| P29 | 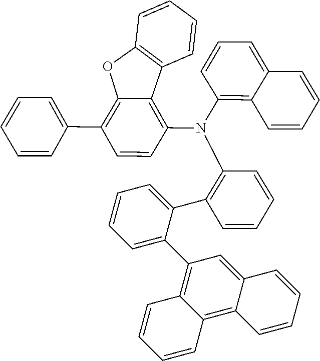 [SMILES: c1ccc(-c2ccc(N(c3ccccc3-c3ccccc3-c3cc4ccccc4c4ccccc34)c3cccc4ccccc34)c3c2oc2ccccc23)cc1] | 19.4 g (68%) | 713.27 |
| | Compound 397 | | |
| P30 | 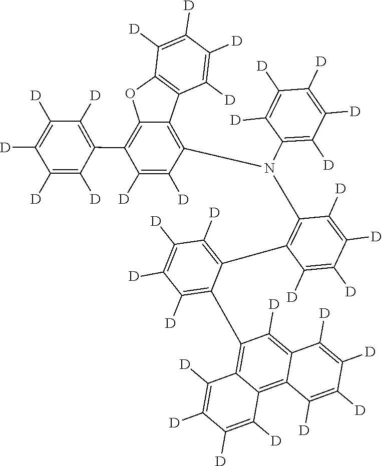 [SMILES: [2H]c1c([2H])c([2H])c(-c2c([2H])c([2H])c(N(c3c([2H])c([2H])c([2H])c([2H])c3[2H])c3c([2H])c([2H])c([2H])c([2H])c3-c3c([2H])c([2H])c([2H])c([2H])c3-c3c([2H])c4c([2H])c([2H])c([2H])c([2H])c4c4c([2H])c([2H])c([2H])c([2H])c34)c3c2oc2c([2H])c([2H])c([2H])c([2H])c23)c([2H])c1[2H]] | 18.4 g (66%) | 696.46 |
| | Compound 426 | | |
| P31 | 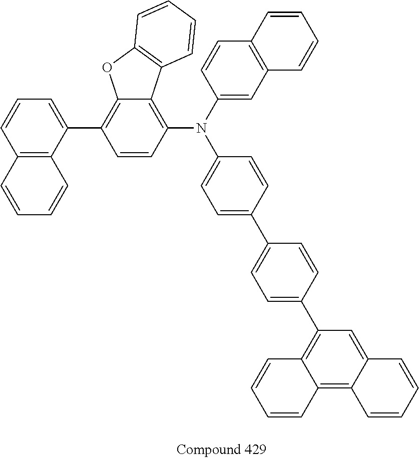 [SMILES: c1ccc2cc(N(c3ccc(-c4ccc(-c5cc6ccccc6c6ccccc56)cc4)cc3)c3ccc(-c4cccc5ccccc45)c4oc5ccccc5c34)ccc2c1] | 22.0 g (72%) | 763.29 |
| | Compound 429 | | |
| P32 | 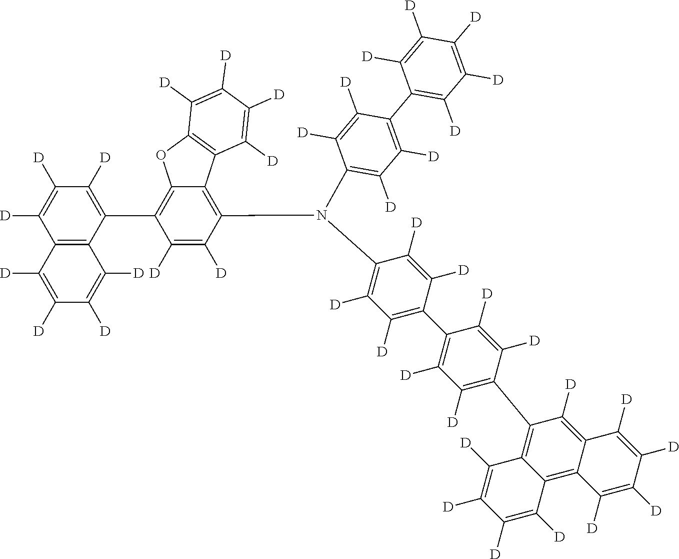 [SMILES: [2H]c1c([2H])c([2H])c(-c2c([2H])c([2H])c(N(c3c([2H])c([2H])c(-c4c([2H])c([2H])c(-c5c([2H])c6c([2H])c([2H])c([2H])c([2H])c6c6c([2H])c([2H])c([2H])c([2H])c56)c([2H])c4[2H])c([2H])c3[2H])c3c([2H])c([2H])c(-c4c([2H])c([2H])c([2H])c5c([2H])c([2H])c([2H])c([2H])c45)c4oc5c([2H])c([2H])c([2H])c([2H])c5c34)c([2H])c2[2H])c([2H])c1[2H]] | 23.5 g (71%) | 828.55 |
| | Compound 473 | | |
| P33 | 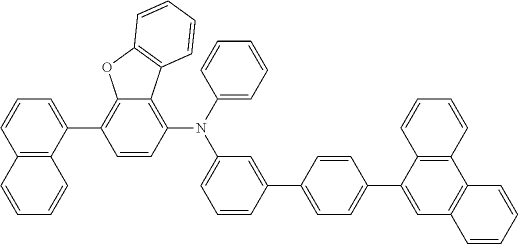 [SMILES: c1ccc(N(c2cccc(-c3ccc(-c4cc5ccccc5c5ccccc45)cc3)c2)c2ccc(-c3cccc4ccccc34)c3oc4ccccc4c23)cc1] | 20.8 g (73%) | 713.27 |
| | Compound 474 | | |
| P34 | 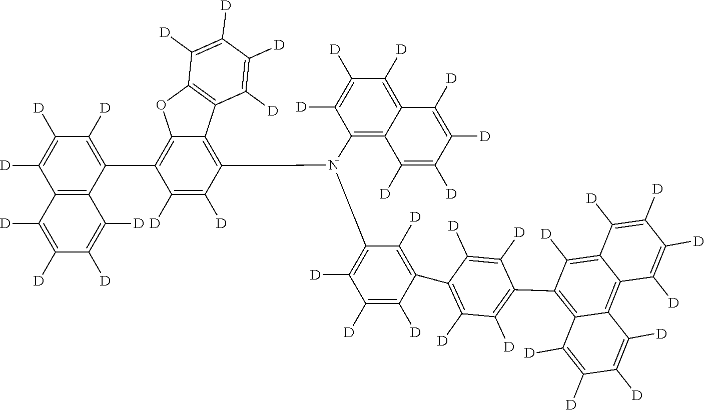 [SMILES: [2H]c1c([2H])c(-c2c([2H])c([2H])c(-c3c([2H])c4c([2H])c([2H])c([2H])c([2H])c4c4c([2H])c([2H])c([2H])c([2H])c34)c([2H])c2[2H])c([2H])c(N(c2c([2H])c([2H])c([2H])c3c([2H])c([2H])c([2H])c([2H])c23)c2c([2H])c([2H])c(-c3c([2H])c([2H])c([2H])c4c([2H])c([2H])c([2H])c([2H])c34)c3oc4c([2H])c([2H])c([2H])c([2H])c4c23)c1[2H]] | 22.4 g (70%) | 800.52 |
| | Compound 520 | | |
| P35 | 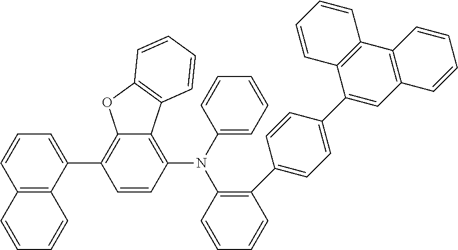 [SMILES: c1ccc(N(c2ccccc2-c2ccc(-c3cc4ccccc4c4ccccc34)cc2)c2ccc(-c3cccc4ccccc34)c3oc4ccccc4c23)cc1] | 20.3 g (71%) | 713.27 |
| | Compound 521 | | |
| P36 | 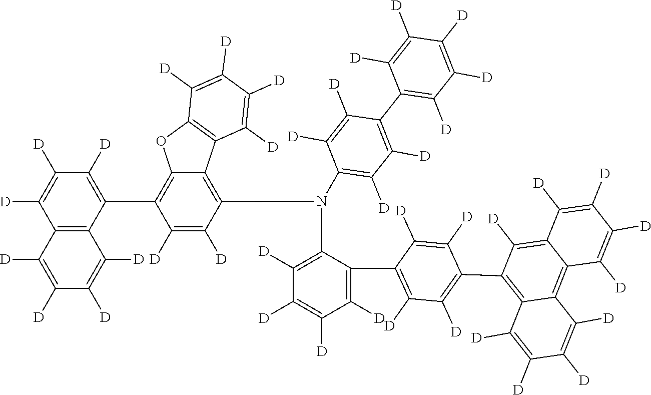 [SMILES: [2H]c1c([2H])c([2H])c(-c2c([2H])c([2H])c(N(c3c([2H])c([2H])c([2H])c([2H])c3-c3c([2H])c([2H])c(-c4c([2H])c5c([2H])c([2H])c([2H])c([2H])c5c5c([2H])c([2H])c([2H])c([2H])c45)c([2H])c3[2H])c3c([2H])c([2H])c(-c4c([2H])c([2H])c([2H])c5c([2H])c([2H])c([2H])c([2H])c45)c4oc5c([2H])c([2H])c([2H])c([2H])c5c34)c([2H])c2[2H])c([2H])c1[2H]] | 22.6 g (68%) | 828.55 |
| | Compound 547 | | |
| P37 | 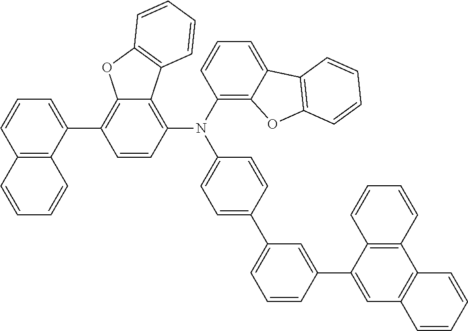 [SMILES: c1cc(-c2ccc(N(c3cccc4c3oc3ccccc34)c3ccc(-c4cccc5ccccc45)c4oc5ccccc5c34)cc2)cc(-c2cc3ccccc3c3ccccc23)c1] | 22.2 g (69%) | 803.28 |
| | Compound 552 | | |
| P38 | 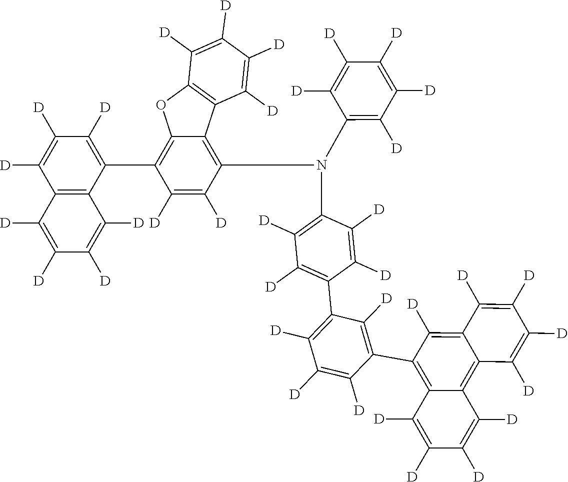 [SMILES: [2H]c1c([2H])c([2H])c(N(c2c([2H])c([2H])c(-c3c([2H])c([2H])c([2H])c(-c4c([2H])c5c([2H])c([2H])c([2H])c([2H])c5c5c([2H])c([2H])c([2H])c([2H])c45)c3[2H])c([2H])c2[2H])c2c([2H])c([2H])c(-c3c([2H])c([2H])c([2H])c4c([2H])c([2H])c([2H])c([2H])c34)c3oc4c([2H])c([2H])c([2H])c([2H])c4c23)c([2H])c1[2H]] | 20.1 g (67%) | 748.49 |
| | Compound 578 | | |
| P39 | 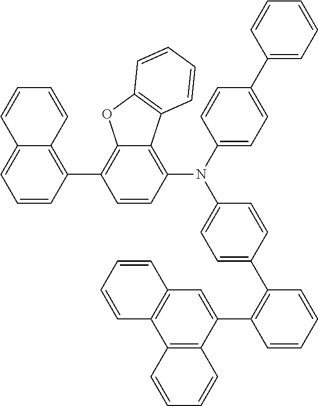 [SMILES: c1ccc(-c2ccc(N(c3ccc(-c4ccccc4-c4cc5ccccc5c5ccccc45)cc3)c3ccc(-c4cccc5ccccc45)c4oc5ccccc5c34)cc2)cc1] | 20.9 g (66%) | 789.30 |
| | Compound 587 | | |
| P40 | 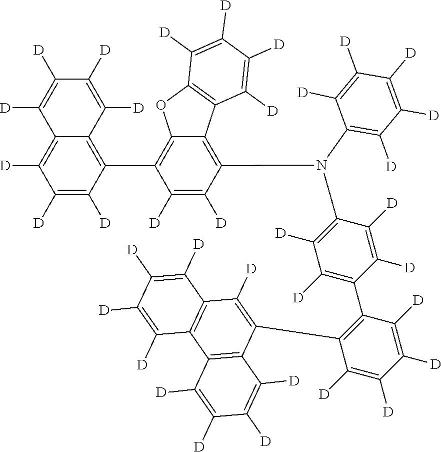 [SMILES: [2H]c1c([2H])c([2H])c(N(c2c([2H])c([2H])c(-c3c([2H])c([2H])c([2H])c([2H])c3-c3c([2H])c4c([2H])c([2H])c([2H])c([2H])c4c4c([2H])c([2H])c([2H])c([2H])c34)c([2H])c2[2H])c2c([2H])c([2H])c(-c3c([2H])c([2H])c([2H])c4c([2H])c([2H])c([2H])c([2H])c34)c3oc4c([2H])c([2H])c([2H])c([2H])c4c23)c([2H])c1[2H]] | 20.4 g (68%) | 748.49 |
| | Compound 609 | | |
| P41 | 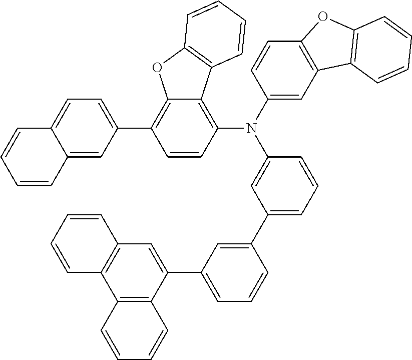 [SMILES: c1cc(-c2cccc(N(c3ccc4oc5ccccc5c4c3)c3ccc(-c4ccc5ccccc5c4)c4oc5ccccc5c34)c2)cc(-c2cc3ccccc3c3ccccc23)c1] | 21.2 g (66%) | 803.28 |
| | Compound 616 | | |
| P42 | 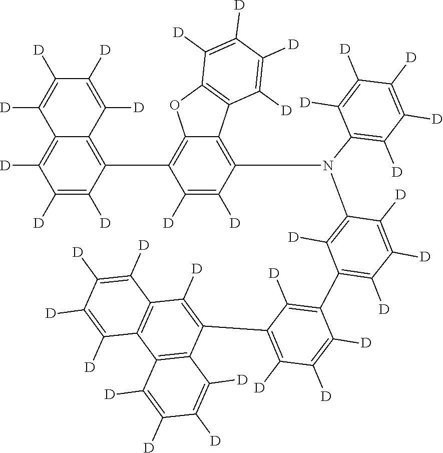 [SMILES: [2H]c1c([2H])c([2H])c(N(c2c([2H])c([2H])c([2H])c(-c3c([2H])c([2H])c([2H])c(-c4c([2H])c5c([2H])c([2H])c([2H])c([2H])c5c5c([2H])c([2H])c([2H])c([2H])c45)c3[2H])c2[2H])c2c([2H])c([2H])c(-c3c([2H])c([2H])c([2H])c4c([2H])c([2H])c([2H])c([2H])c34)c3oc4c([2H])c([2H])c([2H])c([2H])c4c23)c([2H])c1[2H]] | 20.1 g (67%) | 748.49 |
| | Compound 640 | | |
| P43 | 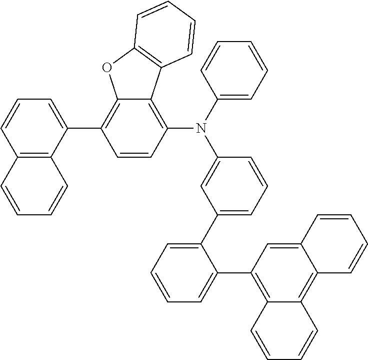 [SMILES: c1ccc(N(c2cccc(-c3ccccc3-c3cc4ccccc4c4ccccc34)c2)c2ccc(-c3cccc4ccccc34)c3oc4ccccc4c23)cc1] | 17.7 g (62%) | 713.27 |
| | Compound 641 | | |
| P44 | 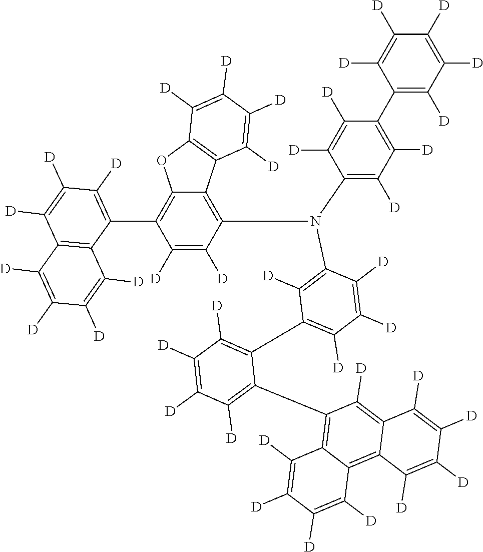 [SMILES: [2H]c1c([2H])c([2H])c(-c2c([2H])c([2H])c(N(c3c([2H])c([2H])c([2H])c(-c4c([2H])c([2H])c([2H])c([2H])c4-c4c([2H])c5c([2H])c([2H])c([2H])c([2H])c5c5c([2H])c([2H])c([2H])c([2H])c45)c3[2H])c3c([2H])c([2H])c(-c4c([2H])c([2H])c([2H])c5c([2H])c([2H])c([2H])c([2H])c45)c4oc5c([2H])c([2H])c([2H])c([2H])c5c34)c([2H])c2[2H])c([2H])c1[2H]] | 20.2 g (61%) | 828.55 |
| | Compound 671 | | |
| P45 | 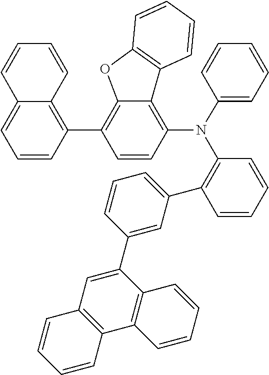 [SMILES: c1ccc(N(c2ccccc2-c2cccc(-c3cc4ccccc4c4ccccc34)c2)c2ccc(-c3cccc4ccccc34)c3oc4ccccc4c23)cc1] | 18.0 g (63%) | 713.27 |
| | Compound 672 | | |
| P46 | 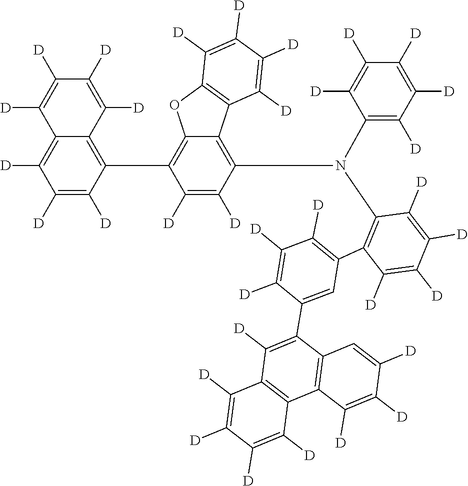 [SMILES: [2H]c1cc2c(-c3cc(-c4c([2H])c([2H])c([2H])c([2H])c4N(c4c([2H])c([2H])c([2H])c([2H])c4[2H])c4c([2H])c([2H])c(-c5c([2H])c([2H])c([2H])c6c([2H])c([2H])c([2H])c([2H])c56)c5oc6c([2H])c([2H])c([2H])c([2H])c6c45)c([2H])c([2H])c3[2H])c([2H])c3c([2H])c([2H])c([2H])c([2H])c3c2c([2H])c1[2H]] | 19.1 g (64%) | 746.48 |
| | Compound 698 | | |
| P47 | 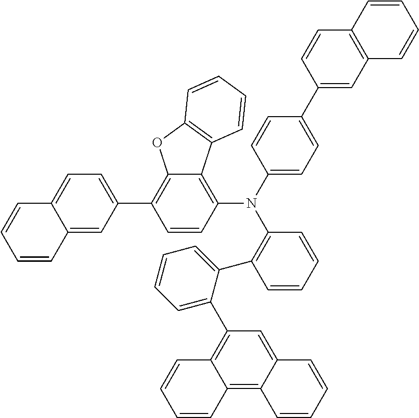 [SMILES: c1ccc(-c2cc3ccccc3c3ccccc23)c(-c2ccccc2N(c2ccc(-c3ccc4ccccc4c3)cc2)c2ccc(-c3ccc4ccccc4c3)c3oc4ccccc4c23)c1] | 20.5 g (61%) | 839.32 |
| | Compound 709 | | |
| P48 | 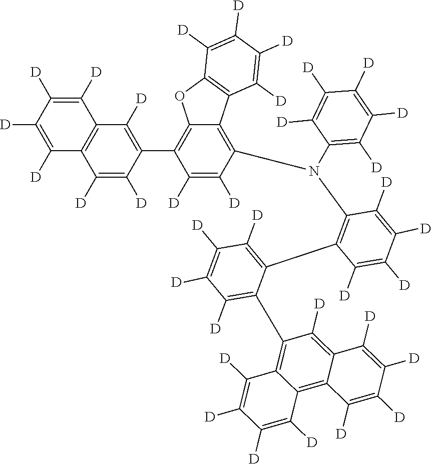 [SMILES: [2H]c1c([2H])c([2H])c(N(c2c([2H])c([2H])c([2H])c([2H])c2-c2c([2H])c([2H])c([2H])c([2H])c2-c2c([2H])c3c([2H])c([2H])c([2H])c([2H])c3c3c([2H])c([2H])c([2H])c([2H])c23)c2c([2H])c([2H])c(-c3c([2H])c([2H])c4c([2H])c([2H])c([2H])c([2H])c4c3[2H])c3oc4c([2H])c([2H])c([2H])c([2H])c4c23)c([2H])c1[2H]] | 18.6 g (62%) | 748.49 |
| | Compound 725 | | |
| P49 | 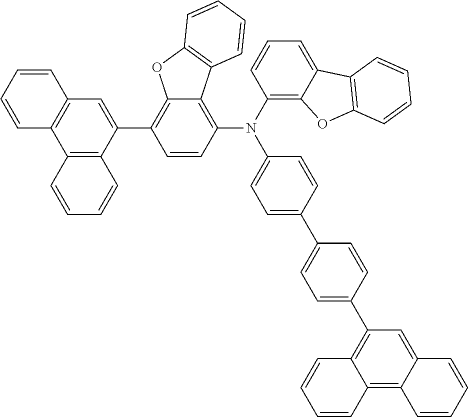 [SMILES: c1ccc2c(c1)cc(-c1ccc(-c3ccc(N(c4cccc5c4oc4ccccc45)c4ccc(-c5cc6ccccc6c6ccccc56)c5oc6ccccc6c45)cc3)cc1)c1ccccc12] | 25.6 g (75%) | 853.30 |
| | Compound 730 | | |
| P50 | 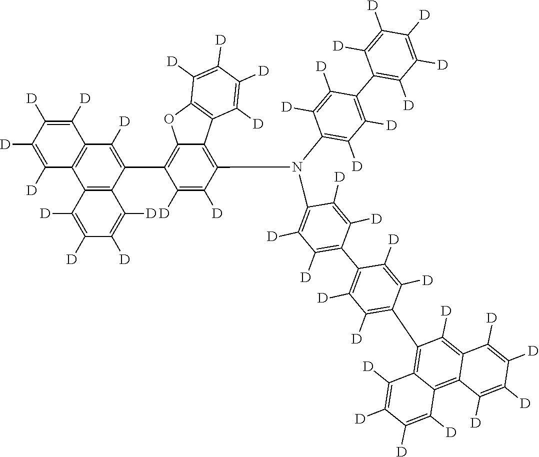 [SMILES: [2H]c1c([2H])c([2H])c(-c2c([2H])c([2H])c(N(c3c([2H])c([2H])c(-c4c([2H])c([2H])c(-c5c([2H])c6c([2H])c([2H])c([2H])c([2H])c6c6c([2H])c([2H])c([2H])c([2H])c56)c([2H])c4[2H])c([2H])c3[2H])c3c([2H])c([2H])c(-c4c([2H])c5c([2H])c([2H])c([2H])c([2H])c5c5c([2H])c([2H])c([2H])c([2H])c45)c4oc5c([2H])c([2H])c([2H])c([2H])c5c34)c([2H])c2[2H])c([2H])c1[2H]] | 25.7 g (73%) | 880.58 |
| | Compound 772 | | |
| P51 | 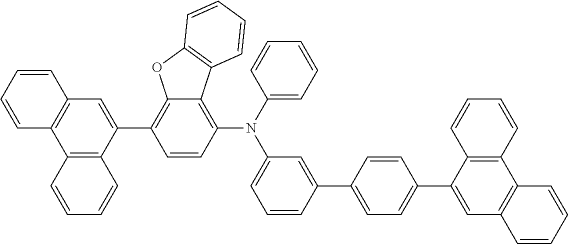 [SMILES: c1ccc(N(c2cccc(-c3ccc(-c4cc5ccccc5c5ccccc45)cc3)c2)c2ccc(-c3cc4ccccc4c4ccccc34)c3oc4ccccc4c23)cc1] | 21.7 g (71%) | 763.29 |
| | Compound 773 | | |
| P52 | 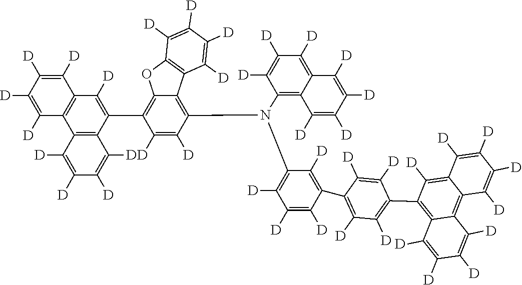 [SMILES: [2H]c1c([2H])c(-c2c([2H])c([2H])c(-c3c([2H])c4c([2H])c([2H])c([2H])c([2H])c4c4c([2H])c([2H])c([2H])c([2H])c34)c([2H])c2[2H])c([2H])c(N(c2c([2H])c([2H])c([2H])c3c([2H])c([2H])c([2H])c([2H])c23)c2c([2H])c([2H])c(-c3c([2H])c4c([2H])c([2H])c([2H])c([2H])c4c4c([2H])c([2H])c([2H])c([2H])c34)c3oc4c([2H])c([2H])c([2H])c([2H])c4c23)c1[2H]] | 23.9 g (70%) | 852.55 |
| | Compound 819 | | |
| P53 | 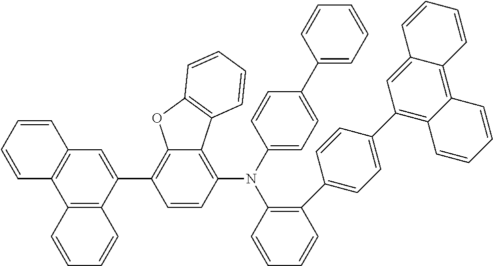 [SMILES: c1ccc(-c2ccc(N(c3ccccc3-c3ccc(-c4cc5ccccc5c5ccccc45)cc3)c3ccc(-c4cc5ccccc5c5ccccc45)c4oc5ccccc5c34)cc2)cc1] | 23.5 g (70%) | 839.32 |
| | Compound 828 | | |
| P54 | 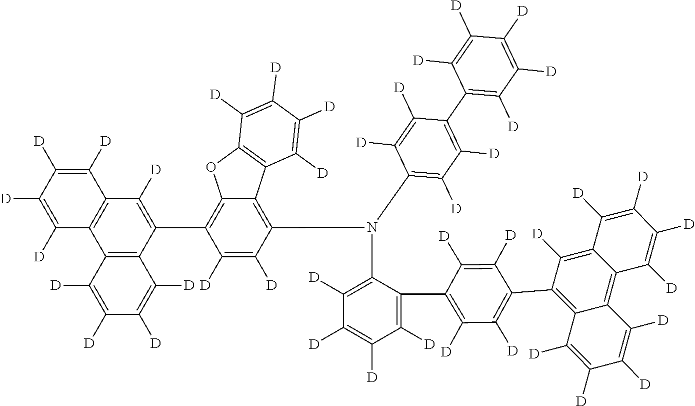 [SMILES: [2H]c1c([2H])c([2H])c(-c2c([2H])c([2H])c(N(c3c([2H])c([2H])c([2H])c([2H])c3-c3c([2H])c([2H])c(-c4c([2H])c5c([2H])c([2H])c([2H])c([2H])c5c5c([2H])c([2H])c([2H])c([2H])c45)c([2H])c3[2H])c3c([2H])c([2H])c(-c4c([2H])c5c([2H])c([2H])c([2H])c([2H])c5c5c([2H])c([2H])c([2H])c([2H])c45)c4oc5c([2H])c([2H])c([2H])c([2H])c5c34)c([2H])c2[2H])c([2H])c1[2H]] | 24.3 g (69%) | 880.58 |
| | Compound 846 | | |
| P55 | 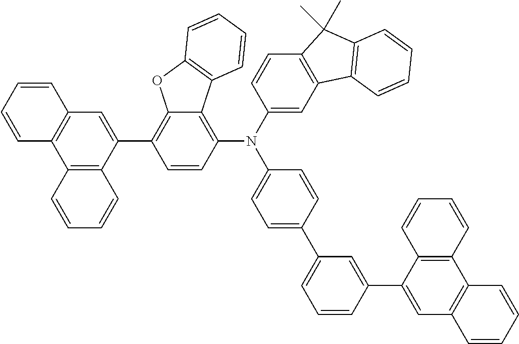 [SMILES: CC1(C)c2ccccc2-c2cc(N(c3ccc(-c4cccc(-c5cc6ccccc6c6ccccc56)c4)cc3)c3ccc(-c4cc5ccccc5c5ccccc45)c4oc5ccccc5c34)ccc21] | 23.6 g (67%) | 879.35 |
| | Compound 853 | | |
| P56 | 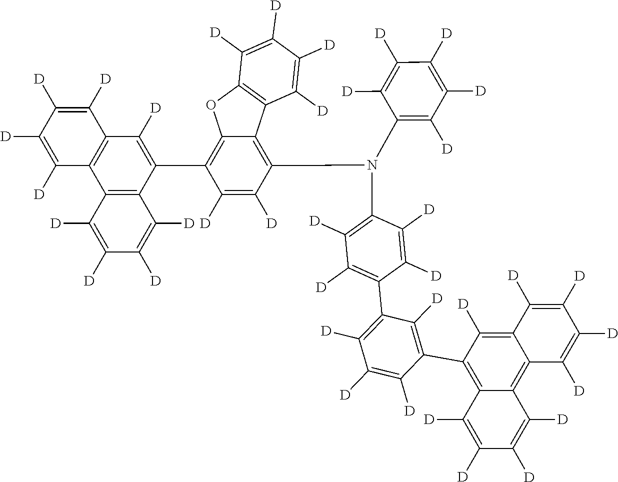 [SMILES: [2H]c1c([2H])c([2H])c(N(c2c([2H])c([2H])c(-c3c([2H])c([2H])c([2H])c(-c4c([2H])c5c([2H])c([2H])c([2H])c([2H])c5c5c([2H])c([2H])c([2H])c([2H])c45)c3[2H])c([2H])c2[2H])c2c([2H])c([2H])c(-c3c([2H])c4c([2H])c([2H])c([2H])c([2H])c4c4c([2H])c([2H])c([2H])c([2H])c34)c3oc4c([2H])c([2H])c([2H])c([2H])c4c23)c([2H])c1[2H]] | 21.8 g (68%) | 800.52 |
| | Compound 877 | | |
| P57 | 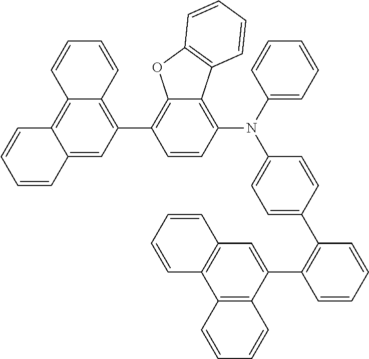 [SMILES: c1ccc(N(c2ccc(-c3ccccc3-c3cc4ccccc4c4ccccc34)cc2)c2ccc(-c3cc4ccccc4c4ccccc34)c3oc4ccccc4c23)cc1] | 20.2 g (66%) | 763.29 |
| | Compound 878 | | |
| P58 | 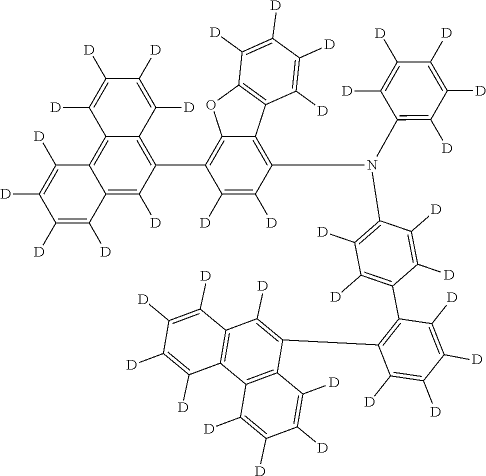 [SMILES: [2H]c1c([2H])c([2H])c(N(c2c([2H])c([2H])c(-c3c([2H])c([2H])c([2H])c([2H])c3-c3c([2H])c4c([2H])c([2H])c([2H])c([2H])c4c4c([2H])c([2H])c([2H])c([2H])c34)c([2H])c2[2H])c2c([2H])c([2H])c(-c3c([2H])c4c([2H])c([2H])c([2H])c([2H])c4c4c([2H])c([2H])c([2H])c([2H])c34)c3oc4c([2H])c([2H])c([2H])c([2H])c4c23)c([2H])c1[2H]] | 21.5 g (67%) | 800.52 |
| | Compound 908 | | |
| P59 | 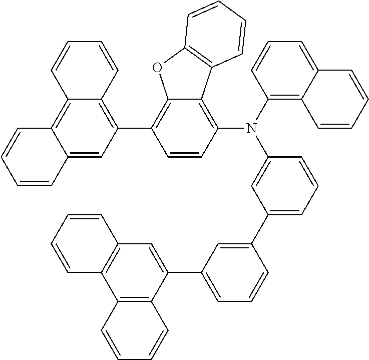 [SMILES: c1cc(-c2cccc(N(c3cccc4ccccc34)c3ccc(-c4cc5ccccc5c5ccccc45)c4oc5ccccc5c34)c2)cc(-c2cc3ccccc3c3ccccc23)c1] | 21.2 g (65%) | 813.30 |
| | Compound 910 | | |
| P60 | 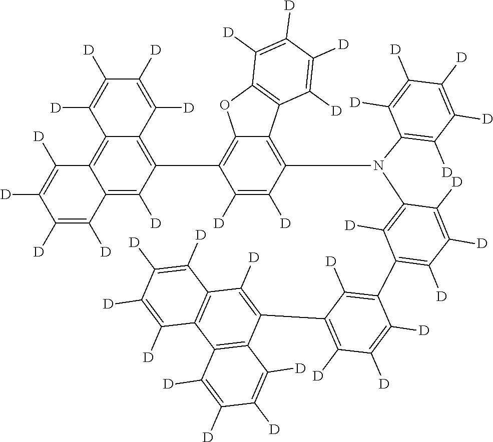 [SMILES: [2H]c1c([2H])c([2H])c(N(c2c([2H])c([2H])c([2H])c(-c3c([2H])c([2H])c([2H])c(-c4c([2H])c5c([2H])c([2H])c([2H])c([2H])c5c5c([2H])c([2H])c([2H])c([2H])c45)c3[2H])c2[2H])c2c([2H])c([2H])c(-c3c([2H])c4c([2H])c([2H])c([2H])c([2H])c4c4c([2H])c([2H])c([2H])c([2H])c34)c3oc4c([2H])c([2H])c([2H])c([2H])c4c23)c([2H])c1[2H]] | 21.8 g (68%) | 800.52 |
| | Compound 939 | | |
| P61 | 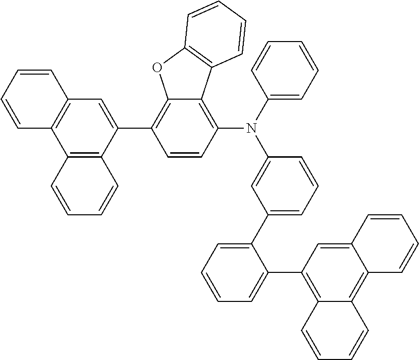 [SMILES: c1ccc(N(c2cccc(-c3ccccc3-c3cc4ccccc4c4ccccc34)c2)c2ccc(-c3cc4ccccc4c4ccccc34)c3oc4ccccc4c23)cc1] | 18.6 g (61%) | 763.29 |
| | Compound 940 | | |
| P62 | 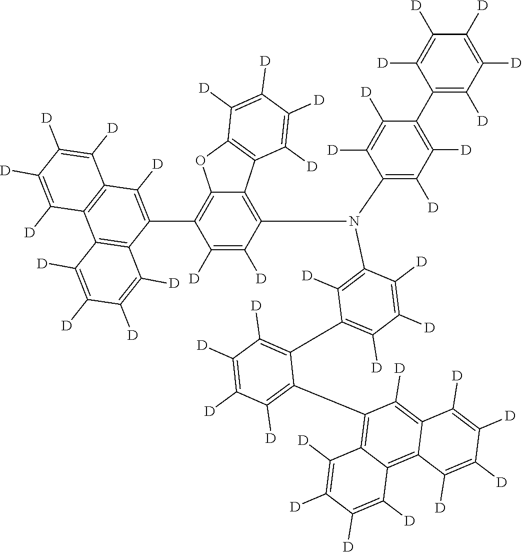 [SMILES: [2H]c1c([2H])c([2H])c(-c2c([2H])c([2H])c(N(c3c([2H])c([2H])c([2H])c(-c4c([2H])c([2H])c([2H])c([2H])c4-c4c([2H])c5c([2H])c([2H])c([2H])c([2H])c5c5c([2H])c([2H])c([2H])c([2H])c45)c3[2H])c3c([2H])c([2H])c(-c4c([2H])c5c([2H])c([2H])c([2H])c([2H])c5c5c([2H])c([2H])c([2H])c([2H])c45)c4oc5c([2H])c([2H])c([2H])c([2H])c5c34)c([2H])c2[2H])c([2H])c1[2H]] | 21.2 g (60%) | 880.58 |
| | Compound 970 | | |
| P63 | 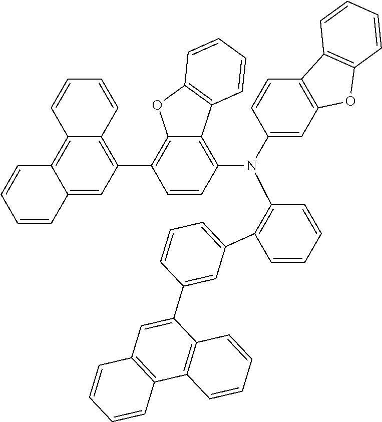 [SMILES: c1cc(-c2ccccc2N(c2ccc3c(c2)oc2ccccc23)c2ccc(-c3cc4ccccc4c4ccccc34)c3oc4ccccc4c23)cc(-c2cc3ccccc3c3ccccc23)c1] | 21.2 g (62%) | 853.30 |
| | Compound 976 | | |
| P64 | 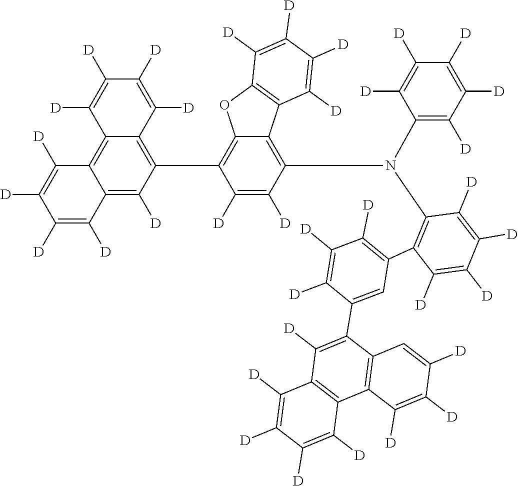 [SMILES: [2H]c1cc2c(-c3cc(-c4c([2H])c([2H])c([2H])c([2H])c4N(c4c([2H])c([2H])c([2H])c([2H])c4[2H])c4c([2H])c([2H])c(-c5c([2H])c6c([2H])c([2H])c([2H])c([2H])c6c6c([2H])c([2H])c([2H])c([2H])c56)c5oc6c([2H])c([2H])c([2H])c([2H])c6c45)c([2H])c([2H])c3[2H])c([2H])c3c([2H])c([2H])c([2H])c([2H])c3c2c([2H])c1[2H]] | 20.5 g (64%) | 798.51 |
| | Compound 997 | | |
| P65 | 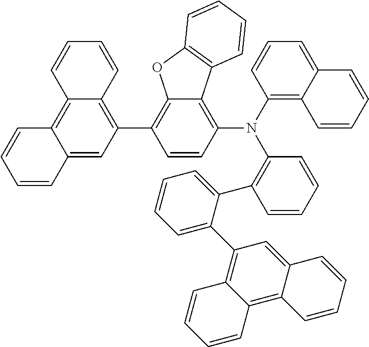 [SMILES: c1ccc(-c2cc3ccccc3c3ccccc23)c(-c2ccccc2N(c2cccc3ccccc23)c2ccc(-c3cc4ccccc4c4ccccc34)c3oc4ccccc4c23)c1] | 20.2 g (62%) | 813.30 |
| | Compound 999 | | |
| P66 | 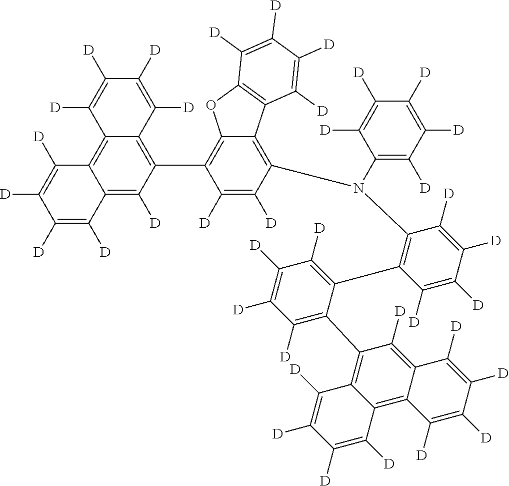 [SMILES: [2H]c1c([2H])c([2H])c(N(c2c([2H])c([2H])c([2H])c([2H])c2Cc2c([2H])c([2H])c([2H])c([2H])c2-c2c([2H])c3c([2H])c([2H])c([2H])c([2H])c3c3c([2H])c([2H])c([2H])c([2H])c23)c2c([2H])c([2H])c(-c3c([2H])c4c([2H])c([2H])c([2H])c([2H])c4c4c([2H])c([2H])c([2H])c([2H])c34)c3oc4c([2H])c([2H])c([2H])c([2H])c4c23)c([2H])c1[2H]] | 19.5 g (61%) | 800.52 |
| | Compound 1024 | | |

### [Experimental Example 1] Measurement of HOMO and LUMO

The effect of the compounds of the present disclosure was confirmed through the following experiments, which are provided as representative examples only and are not intended to limit the scope of the experimental examples.

### Experimental Example 1: Simulation Results for Hole Transport Auxiliary Layer

The hole transport auxiliary layer serves to reduce the accumulation of holes at the interface of the emitting layer due to the difference in the HOMO energy levels between the hole transport layer and the emitting layer. For this purpose, it is preferable that the HOMO energy level difference between the hole transport auxiliary layer and the emitting layer be smaller than that between the hole transport auxiliary layer and the hole transport layer. In addition, the hole transport auxiliary layer should have a higher LUMO energy level than that of the emitting layer in order to minimize electron coming from the emitting layer to the hole transport layer.

To confirm whether the organic compound represented by Chemical Formula 1 according to the present disclosure is suitable as a material for the hole transport auxiliary layer, the HOMO energy level (eV) and LUMO energy levels (eV) were calculated using Spartan software (B3LYP DFT 6-31G* by Spartan'16). The results are shown in Table 2 below.

TABLE 2

| | HOMO | LUMO |
|---|---|---|
| Compounds | (calculation) | (calculation) |
| 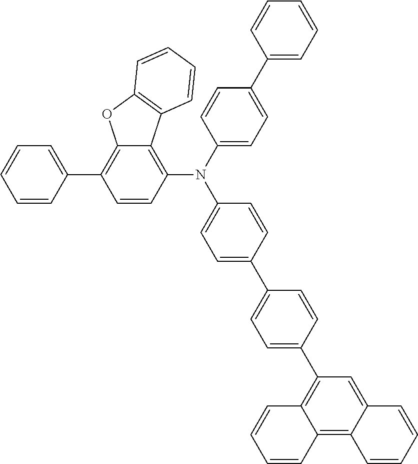 [SMILES: c1ccc(-c2ccc(N(c3ccc(-c4ccc(-c5cc6ccccc6c6ccccc56)cc4)cc3)c3ccc(-c4ccccc4)c4oc5ccccc5c34)cc2)cc1] | −5.02 | −1.18 |
| Compound 17 | | |
| 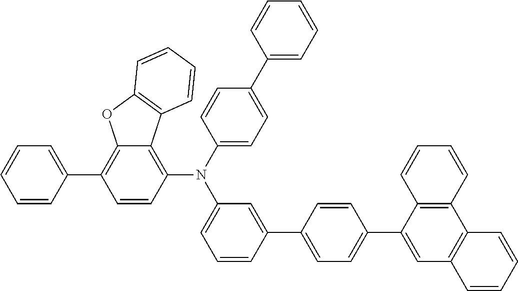 [SMILES: c1ccc(-c2ccc(N(c3cccc(-c4ccc(-c5cc6ccccc6c6ccccc56)cc4)c3)c3ccc(-c4ccccc4)c4oc5ccccc5c34)cc2)cc1] | −5.04 | −1.16 |
| Compound 86 | | |
| 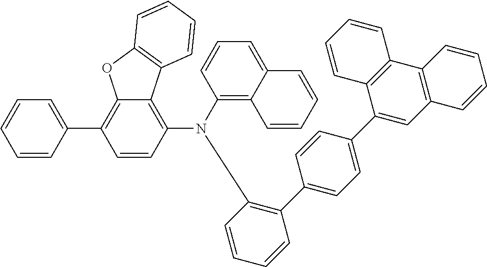 [SMILES: c1ccc(-c2ccc(N(c3ccccc3-c3ccc(-c4cc5ccccc5c5ccccc45)cc3)c3cccc4ccccc34)c3c2oc2ccccc23)cc1] | −5.00 | −1.28 |
| Compound 133 | | |
| 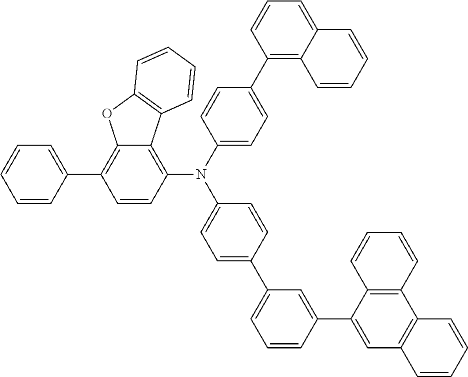 [SMILES: c1ccc(-c2ccc(N(c3ccc(-c4cccc(-c5cc6ccccc6c6ccccc56)c4)cc3)c3ccc(-c4cccc5ccccc45)cc3)c3c2oc2ccccc23)cc1] | −5.04 | −1.18 |
| Compound 199 | | |
| 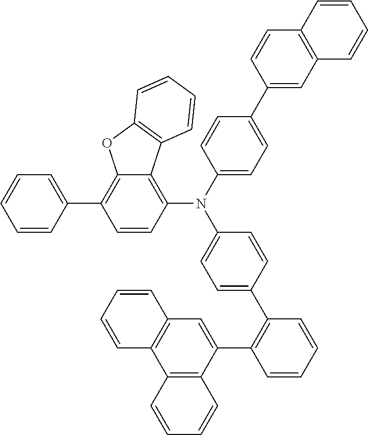 [SMILES: c1ccc(-c2ccc(N(c3ccc(-c4ccc5ccccc5c4)cc3)c3ccc(-c4ccccc4-c4cc5ccccc5c5ccccc45)cc3)c3c2oc2ccccc23)cc1] | −5.03 | −1.18 |
| Compound 258 | | |
| 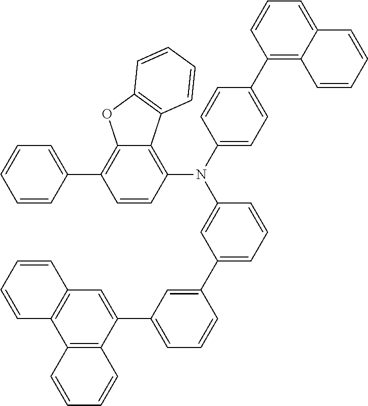 [SMILES: c1ccc(-c2ccc(N(c3ccc(-c4cccc5ccccc45)cc3)c3cccc(-c4cccc(-c5cc6ccccc6c6ccccc56)c4)c3)c3c2oc2ccccc23)cc1] | −5.06 | −1.16 |
| Compound 296 | | |
| 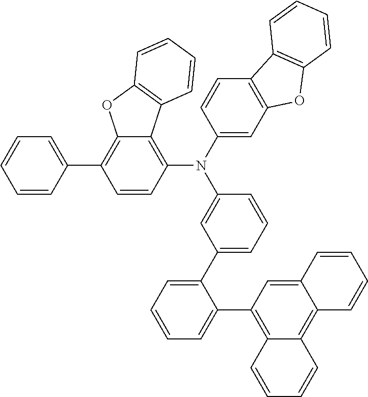 [SMILES: c1ccc(-c2ccc(N(c3cccc(-c4ccccc4-c4cc5ccccc5c5ccccc45)c3)c3ccc4c(c3)oc3ccccc34)c3c2oc2ccccc23)cc1] | −5.00 | −1.19 |
| Compound 1026 | | |
| 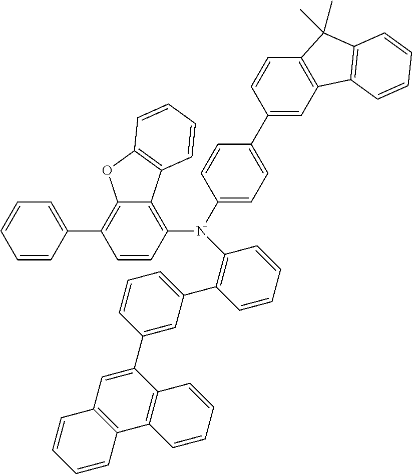 [SMILES: CC1(C)c2ccccc2-c2cc(-c3ccc(N(c4ccccc4-c4cccc(-c5cc6ccccc6c6ccccc56)c4)c4ccc(-c5ccccc5)c5oc6ccccc6c45)cc3)ccc21] | −4.96 | −1.13 |
| Compound 1027 | | |
| 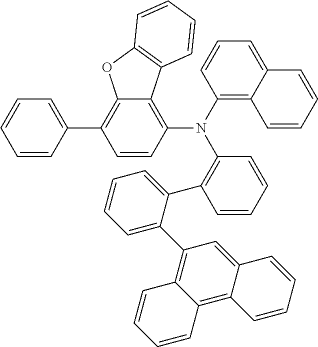 [SMILES: c1ccc(-c2ccc(N(c3ccccc3-c3ccccc3-c3cc4ccccc4c4ccccc34)c3cccc4ccccc34)c3c2oc2ccccc23)cc1] | −4.97 | −1.26 |
| Compound 397 | | |
| 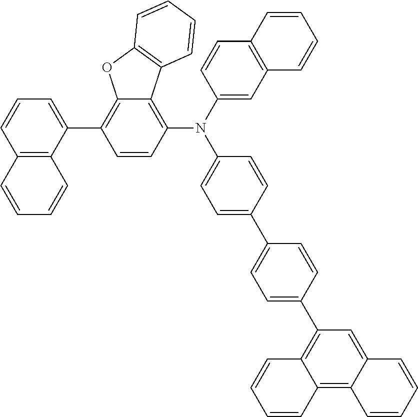 [SMILES: c1ccc2cc(N(c3ccc(-c4ccc(-c5cc6ccccc6c6ccccc56)cc4)cc3)c3ccc(-c4cccc5ccccc45)c4oc5ccccc5c34)ccc2c1] | −4.98 | −1.21 |
| Compound 429 | | |
| 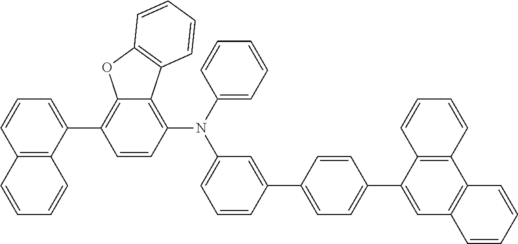 [SMILES: c1ccc(N(c2cccc(-c3ccc(-c4cc5ccccc5c5ccccc45)cc3)c2)c2ccc(-c3cccc4ccccc34)c3oc4ccccc4c23)cc1] | −5.08 | −1.16 |
| Compound 474 | | |
| 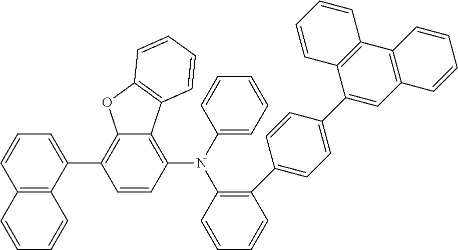 [SMILES: c1ccc(N(c2ccccc2-c2ccc(-c3cc4ccccc4c4ccccc34)cc2)c2ccc(-c3cccc4ccccc34)c3oc4ccccc4c23)cc1] | −5.07 | −1.14 |
| Compound 521 | | |
| 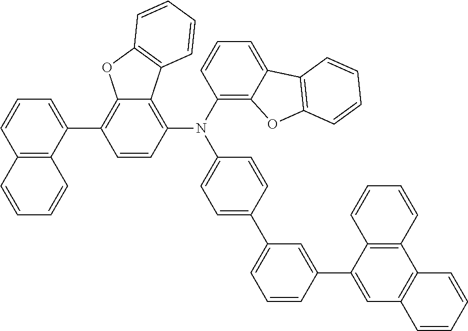 [SMILES: c1cc(-c2ccc(N(c3cccc4c3oc3ccccc34)c3ccc(-c4cccc5ccccc45)c4oc5ccccc5c34)cc2)cc(-c2cc3ccccc3c3ccccc23)c1] | −5.05 | −1.19 |
| Compound 552 | | |
| 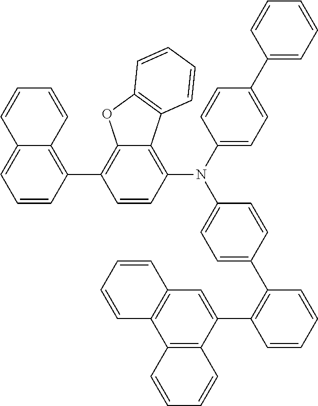 [SMILES: c1ccc(-c2ccc(N(c3ccc(-c4ccccc4-c4cc5ccccc5c5ccccc45)cc3)c3ccc(-c4cccc5ccccc45)c4oc5ccccc5c34)cc2)cc1] | −4.95 | −1.20 |
| Compound 587 | | |
| 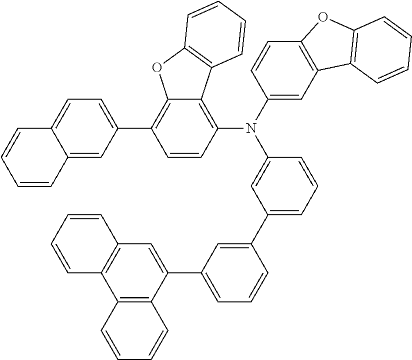 [SMILES: c1cc(-c2cccc(N(c3ccc4oc5ccccc5c4c3)c3ccc(-c4ccc5ccccc5c4)c4oc5ccccc5c34)c2)cc(-c2cc3ccccc3c3ccccc23)c1] | −5.01 | −1.34 |
| Compound 616 | | |
| 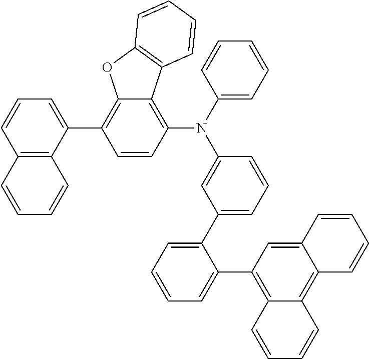 [SMILES: c1ccc(N(c2cccc(-c3ccccc3-c3cc4ccccc4c4ccccc34)c2)c2ccc(-c3cccc4ccccc34)c3oc4ccccc4c23)cc1] | −4.99 | −1.16 |
| Compound 641 | | |
| 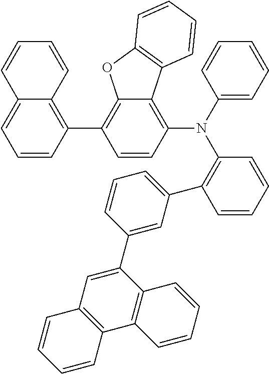 [SMILES: c1ccc(N(c2ccccc2-c2cccc(-c3cc4ccccc4c4ccccc34)c2)c2ccc(-c3cccc4ccccc34)c3oc4ccccc4c23)cc1] | −5.05 | −1.12 |
| Compound 672 | | |
| 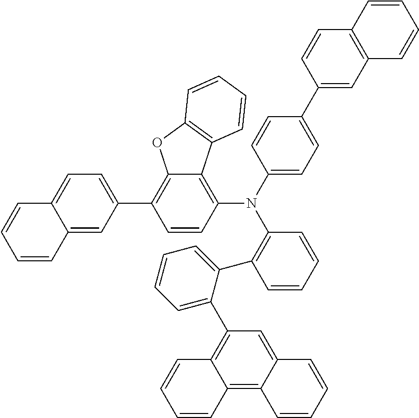 [SMILES: c1ccc(-c2cc3ccccc3c3ccccc23)c(-c2ccccc2N(c2ccc(-c3ccc4ccccc4c3)cc2)c2ccc(-c3ccc4ccccc4c3)c3oc4ccccc4c23)c1] | −5.02 | −1.31 |
| Compound 709 | | |
| 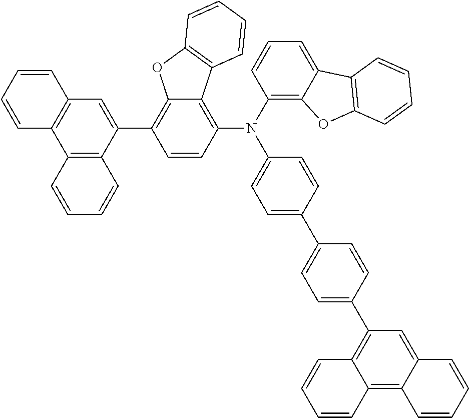 [SMILES: c1ccc2c(c1)cc(-c1ccc(-c3ccc(N(c4cccc5c4oc4ccccc45)c4ccc(-c5cc6ccccc6c6ccccc56)c5oc6ccccc6c45)cc3)cc1)c1ccccc12] | −5.06 | −1.22 |
| Compound 730 | | |
| 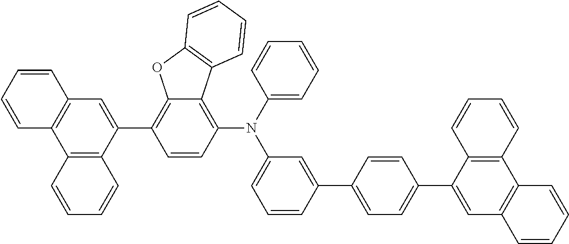 [SMILES: c1ccc(N(c2cccc(-c3ccc(-c4cc5ccccc5c5ccccc45)cc3)c2)c2ccc(-c3cc4ccccc4c4ccccc34)c3oc4ccccc4c23)cc1] | −5.09 | −1.19 |
| Compound 773 | | |
| 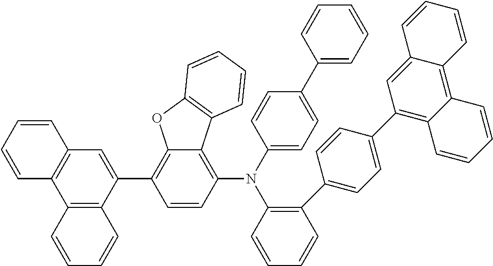 [SMILES: c1ccc(-c2ccc(N(c3ccccc3-c3ccc(-c4cc5ccccc5c5ccccc45)cc3)c3ccc(-c4cc5ccccc5c5ccccc45)c4oc5ccccc5c34)cc2)cc1] | −5.06 | −1.17 |
| Compound 828 | | |
| 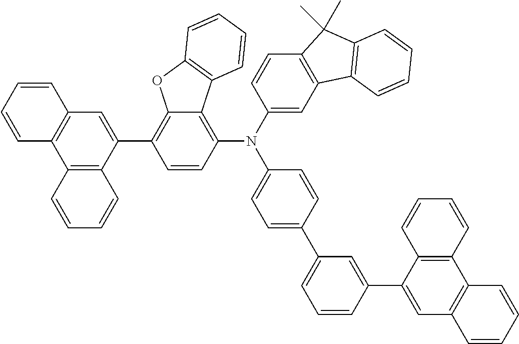 [SMILES: CC1(C)c2ccccc2-c2cc(N(c3ccc(-c4cccc(-c5cc6ccccc6c6ccccc56)c4)cc3)c3ccc(-c4cc5ccccc5c5ccccc45)c4oc5ccccc5c34)ccc21] | −4.98 | −1.18 |
| Compound 853 | | |
| 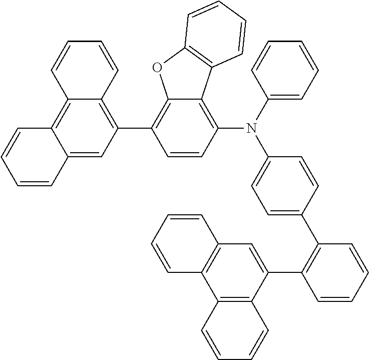 [SMILES: c1ccc(N(c2ccc(-c3ccccc3-c3cc4ccccc4c4ccccc34)cc2)c2ccc(-c3cc4ccccc4c4ccccc34)c3oc4ccccc4c23)cc1] | −4.99 | −1.22 |
| Compound 878 | | |
| 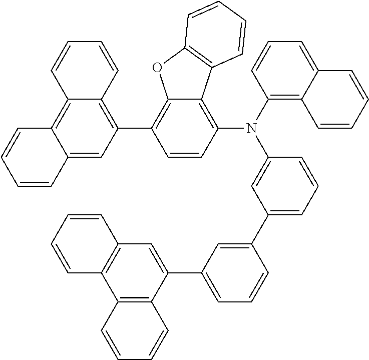 [SMILES: c1cc(-c2cccc(N(c3cccc4ccccc34)c3ccc(-c4cc5ccccc5c5ccccc45)c4oc5ccccc5c34)c2)cc(-c2cc3ccccc3c3ccccc23)c1] | −5.02 | −1.32 |
| Compound 910 | | |
| 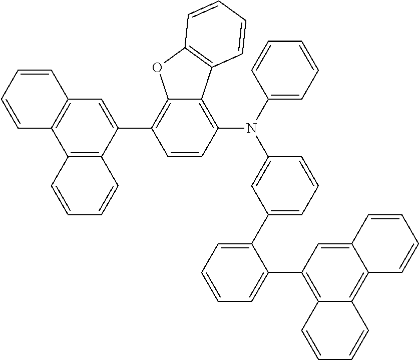 [SMILES: c1ccc(N(c2cccc(-c3ccccc3-c3cc4ccccc4c4ccccc34)c2)c2ccc(-c3cc4ccccc4c4ccccc34)c3oc4ccccc4c23)cc1] | −4.98 | −1.23 |
| Compound 940 | | |
| 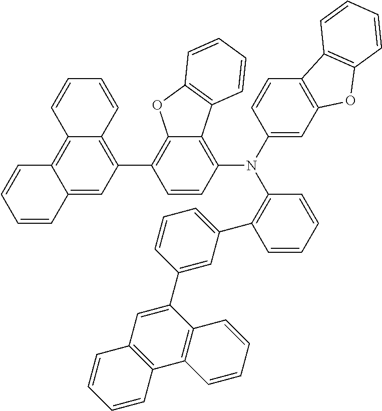 [SMILES: c1cc(-c2ccccc2N(c2ccc3c(c2)oc2ccccc23)c2ccc(-c3cc4ccccc4c4ccccc34)c3oc4ccccc4c23)cc(-c2cc3ccccc3c3ccccc23)c1] | −5.02 | −1.16 |
| Compound 976 | | |
| 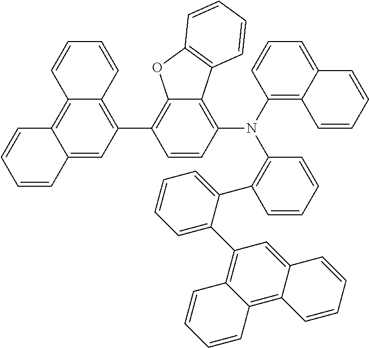 [SMILES: c1ccc(-c2cc3ccccc3c3ccccc23)c(-c2ccccc2N(c2cccc3ccccc23)c2ccc(-c3cc4ccccc4c4ccccc34)c3oc4ccccc4c23)c1] | −4.96 | −1.24 |
| Compound 999 | | |

### [Example 1] Manufacturing of Organic Light Emitting Diode (Blue Emitting Layer)

A substrate on which an ITO (100 nm) serving as the first electrode (anode) of the organic light emitting diode was stacked was patterned by a photolithography process to define the regions of the second electrode (cathode) and the first electrode (anode), and the insulating layer. Subsequently, the surface of the first electrode (ITO) was treated with UV-ozone and O₂:N₂ plasma to enhance its work function and to clean the surface.

Next, on the anode, a mixture of NDP-9 (2-(7-Dicyanomethylene-1,3,4,5,6,8,9,10-octafluoro-7H-pyren-2-ylidene)-malononitrile) and N4,N4,N4′,N4′-tetra([1,1′-biphenyl]-4-yl)-[1,1′-biphenyl]-4,4′-diamine at a weight ratio of 3:97 was vacuum-deposited to form a hole injection layer (HIL) with a thickness of 10 nm. Subsequently, on the hole injection layer, N4,N4,N4′,N4′-tetra([1,1′-biphenyl]-4-yl)-[1,1′-biphenyl]-4,4′-diamine (N4,N4,N4′,N4′-Tetra([1,1′-biphenyl]-4-yl)-[1,1′-biphenyl]-4,4′-diamine) was vacuum-deposited to form a hole transport layer with a thickness of 100 nm, and on the hole transport layer (HTL), compound 1 was vacuum-deposited to form a hole transport auxiliary layer with a thickness of 15 nm.

On the hole transport auxiliary layer, 9,10-bis(2-naphthyl)anthracene (ADN) as a host and 2,12-di-tert-butyl-5,9-bis(4-(tert-butyl)phenyl)-7-(3,5-di-tert-butylphenyl)-5,9-dihydro-5,9-diaza-13b-boranaphtho[3,2,1-de]anthracene (t-DABNA-dtB) as a dopant were vacuum-deposited to form a blue emitting layer with a thickness of 25 nm, wherein a mixing weight ratio of the host:dopant was 97:3. On the blue emitting layer, a mixture of 2-(4-(9,10-di(naphthalene-2-yl)anthracen-2-yl)phenyl)-1-phenyl-1H-benzo[d]imidazole and Liq at a weight ratio of 1:1 was vacuum-deposited to form an electron transport layer (ETL) with a thickness of 25 nm. On the electron transport layer (ETL), an electron injection layer (Liq) was vacuum-deposited to a thickness of 1 nm, and a mixture of magnesium and silver at a weight ratio of 1:4 was vacuum-deposited to form a cathode with a thickness of 16 nm. On the cathode, N4,N4′-bis[4-[bis(3-methylphenyl)amino]phenyl]-N4,N4′-diphenyl-[1,1′-biphenyl]-4,4′-diamine (DNTPD) was vacuum-deposited to form a capping layer with a thickness of 60 nm. An organic light emitting diode was manufactured by bonding a seal cap containing a desiccant using a UV-curable adhesive onto the capping layer, thereby forming an encapsulation layer (or protecting layer) to protect the organic light emitting diode from moisture and oxygen in the atmosphere.

### Examples 2 to 68

Organic light emitting diodes of Examples 2 to 68 were manufactured in the same manner as in Example 1, except that Compound 1 used as the material for the hole transport auxiliary layer in Example 1 was replaced with the compounds described in Table 3 below.

### Comparative Examples 1 and 2

Organic light emitting diodes of Comparative Examples 1 and 2 were each manufactured in the same manner as in Example 1, except that Compound 1 used as the material for the hole transport auxiliary layer in Example 1 was replaced with Compound A and Compound B below. The structures of Compounds A and B, which were used as materials for the hole transport layer in Comparative Examples 1 and 2, are as follow.

Compound A

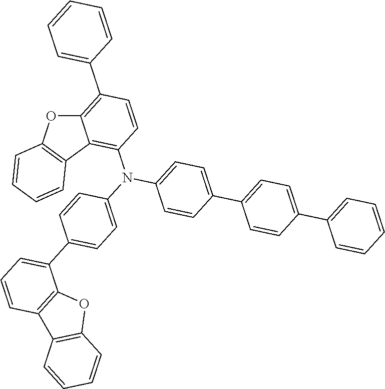 [SMILES: c1ccc(-c2ccc(-c3ccc(N(c4ccc(-c5cccc6c5oc5ccccc56)cc4)c4ccc(-c5ccccc5)c5oc6ccccc6c45)cc3)cc2)cc1]

Compound B

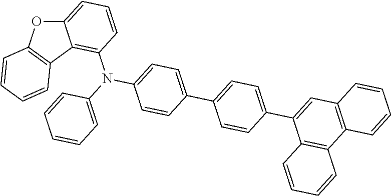 [SMILES: c1ccc(N(c2ccc(-c3ccc(-c4cc5ccccc5c5ccccc45)cc3)cc2)c2cccc3oc4ccccc4c23)cc1]

### [Experimental Example 2] Performance Evaluation of Organic Light Emitting Diode (Blue Devices)

For each of the organic light emitting diode manufactured in Examples 1 to 68 and Comparative Examples 1 and 2, the driving voltage (V) and external quantum efficiency (EQE) (%) were measured by applying a current of 10 mA/cm² using a CS-2000 manufactured by KONICA MINOLTA. In addition, the lifetime (LT95) (hrs) was measured using an M6000 system manufactured by McScience by confirming the time for the luminance to decrease to 95% of the initial luminance under a constant current drive of 10 mA/cm². The measurement results are shown in Table 3 below.

TABLE 3

| | Compound of Hole | Driving | | Lifetime |
|---|---|---|---|---|
| | transporting auxiliary | voltage | | (LT95) |
| Examples | layer | (V) | EQE (%) | (hrs) |
| Example 1 | Compound 1 | 3.50 | 21.3 | 430 |
| Example 2 | Compound 2 | 3.41 | 21.7 | 445 |
| Example 3 | Compound 5 | 3.55 | 13.5 | 432 |
| Example 4 | Compound 14 | 3.41 | 13.8 | 434 |
| Example 5 | Compound 17 | 3.53 | 21.8 | 441 |
| Example 6 | Compound 21 | 3.52 | 22.0 | 445 |
| Example 7 | Compound 22 | 3.43 | 20.2 | 435 |
| Example 8 | Compound 25 | 3.54 | 21.8 | 440 |
| Example 9 | Compound 63 | 3.53 | 21.8 | 483 |
| Example 10 | Compound 66 | 3.52 | 22.0 | 488 |
| Example 11 | Compound 69 | 3.54 | 21.8 | 481 |
| Example 12 | Compound 70 | 3.50 | 19.1 | 432 |
| Example 13 | Compound 71 | 3.43 | 19.0 | 432 |
| Example 14 | Compound 75 | 3.52 | 13.7 | 431 |
| Example 15 | Compound 86 | 3.53 | 19.1 | 430 |
| Example 16 | Compound 90 | 3.54 | 21.0 | 435 |
| Example 17 | Compound 128 | 3.50 | 19.1 | 480 |
| Example 18 | Compound 131 | 3.51 | 13.9 | 485 |
| Example 19 | Compound 132 | 3.48 | 19.1 | 436 |
| Example 20 | Compound 133 | 3.43 | 18.3 | 438 |
| Example 21 | Compound 140 | 3.49 | 19.1 | 432 |
| Example 22 | Compound 153 | 3.46 | 13.0 | 430 |
| Example 23 | Compound 178 | 3.43 | 18.3 | 479 |
| Example 24 | Compound 181 | 3.49 | 19.1 | 485 |
| Example 25 | Compound 182 | 3.51 | 19.1 | 430 |
| Example 26 | Compound 196 | 3.43 | 13.2 | 438 |
| Example 27 | Compound 199 | 3.46 | 18.4 | 436 |
| Example 28 | Compound 244 | 3.51 | 19.1 | 486 |
| Example 29 | Compound 247 | 3.46 | 20.6 | 483 |
| Example 30 | Compound 248 | 3.53 | 19.5 | 438 |
| Example 31 | Compound 1025 | 3.45 | 13.6 | 439 |
| Example 32 | Compound 258 | 3.44 | 19.1 | 430 |
| Example 33 | Compound 286 | 3.53 | 19.5 | 485 |
| Example 34 | Compound 287 | 3.51 | 15.8 | 441 |
| Example 35 | Compound 296 | 3.46 | 15.4 | 433 |
| Example 36 | Compound 325 | 3.51 | 15.8 | 482 |
| Example 37 | Compound 1026 | 3.45 | 11.2 | 438 |
| Example 38 | Compound 364 | 3.50 | 15.9 | 485 |
| Example 39 | Compound 1027 | 3.43 | 11.2 | 429 |
| Example 40 | Compound 395 | 3.46 | 11.2 | 485 |
| Example 41 | Compound 397 | 3.48 | 15.1 | 435 |
| Example 42 | Compound 426 | 3.50 | 15.6 | 480 |
| Example 43 | Compound 427 | 3.52 | 20.3 | 388 |
| Example 44 | Compound 429 | 3.51 | 20.2 | 386 |
| Example 45 | Compound 432 | 3.53 | 13.8 | 390 |
| Example 46 | Compound 447 | 3.54 | 21.3 | 394 |
| Example 47 | Compound 1028 | 3.52 | 21.9 | 396 |
| Example 48 | Compound 473 | 3.53 | 20.9 | 412 |
| Example 49 | Compound 474 | 3.51 | 18.8 | 381 |
| Example 50 | Compound 488 | 3.42 | 13.8 | 383 |
| Example 51 | Compound 520 | 3.49 | 18.3 | 412 |
| Example 52 | Compound 521 | 3.50 | 18.6 | 381 |
| Example 53 | Compound 529 | 3.55 | 19.0 | 383 |
| Example 54 | Compound 547 | 3.54 | 19.0 | 414 |
| Example 55 | Compound 548 | 3.56 | 19.2 | 390 |
| Example 56 | Compound 552 | 3.58 | 13.6 | 383 |
| Example 57 | Compound 578 | 3.56 | 19.2 | 415 |
| Example 58 | Compound 587 | 3.62 | 18.7 | 380 |
| Example 59 | Compound 609 | 3.61 | 18.7 | 415 |
| Example 60 | Compound 616 | 3.64 | 11.7 | 382 |
| Example 61 | Compound 641 | 3.60 | 15.9 | 386 |
| Example 62 | Compound 672 | 3.58 | 15.4 | 381 |
| Example 63 | Compound 707 | 3.61 | 15.7 | 389 |
| Example 64 | Compound 730 | 3.77 | 7.9 | 305 |
| Example 65 | Compound 773 | 3.71 | 9.9 | 347 |
| Example 66 | Compound 828 | 3.78 | 9.0 | 300 |
| Example 67 | Compound 853 | 3.71 | 7.7 | 345 |
| Example 68 | Compound 878 | 3.71 | 9.4 | 340 |
| Comprative | Compound A | 3.82 | 8.1 | 260 |
| Example 1 | | | | |
| Comprative | Compound B | 3.89 | 7.9 | 210 |
| Example 2 | | | | |

The compounds represented by Chemical Formula 1 according to the present disclosure are characterized in that they comprise a phenyl-phenyl-phenanthrene structure and a dibenzofuran moiety bonded to an arylamine, wherein the dibenzofuran moiety further includes phenyl, naphthyl, or phenanthryl groups. Comparative Compound A (Comparative Example 1) does not include the phenyl-phenyl-phenanthrene structure, while Compound B (Comparative Example 2) does not include a phenyl, naphthyl, or phenanthryl groups at the 4-position of the dibenzofuran moiety.

Due to these characteristic structural features, the compounds of the present disclosure represented by Chemical Formula 1 are capable of regulating hole transporting properties, thereby reducing the accumulation of holes at the interface between the hole transporting auxiliary layer and the emitting layer, compared to the comparative compounds that do not satisfy the structure of Chemical Formula 1. This effect may reduce the quenching phenomenon where excitons are quenched by polarons at the interface of the hole transport auxiliary layer and the emitting layer. As shown in Table 3, it was confirmed that the compounds according to the present disclosure can reduce device degradation and improve device stability compared to the compounds of the Comparative Examples, thereby lowering the driving voltage and improving the efficiency and lifetime when applied to a device.

While the embodiments of this specification have been described in detail above, the present specification is not necessarily limited to these embodiments, and various modifications may be made without departing from the technical spirit of this specification. Accordingly, the embodiments disclosed in this specification are for the purpose of illustration and not for limitation of the technical spirit of the present specification, and the scope of the technical spirit of the present specification is not limited by these embodiments. Therefore, the embodiments described above should be understood as illustrative rather than limiting in all aspects.

What is claimed is:

1. An organic compound represented by the following Chemical Formula 1: 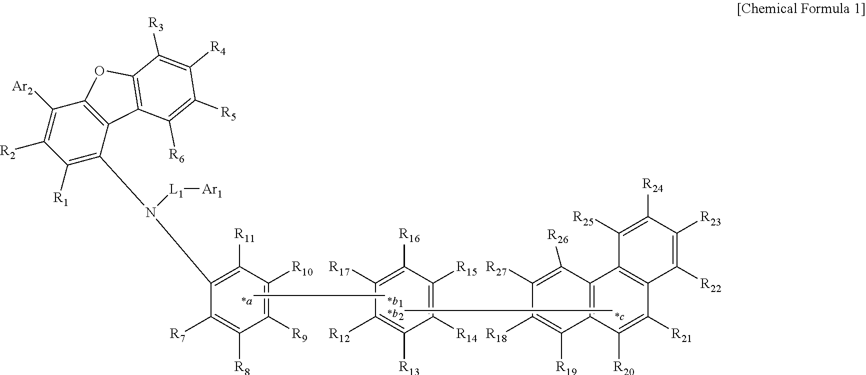
wherein in the chemical formula 1,
L₁ is selected from the group consisting of a single bond, a substituted or unsubstituted arylene group having 6 to 30 carbon atoms, and a substituted or unsubstituted heteroarylene group having 1 to 30 carbon atoms;
Ar₁ and Ar₂ are identical to or different from each other, and are each independently selected from the group consisting of a substituted or unsubstituted aryl group having 6 to 30 carbon atoms, and a substituted or unsubstituted heteroaryl group having 1 to 30 carbon atoms;
R₁ to R₂₇ are identical to or different from each other, and are each independently selected from hydrogen, deuterium, a substituted or unsubstituted alkyl group having 1 to 10 carbon atoms, a substituted or unsubstituted aryl group having 6 to 30 carbon atoms, and a substituted or unsubstituted heteroaryl group having 1 to 30 carbon atoms;
when L₁, Ar₁, Ar₂, and R₁ to R₂₇ are substituted, the substituents are identical to or different from each other, and may be one or more selected from the group consisting of deuterium, a cyano group, a nitro group, a halogen group, a hydroxy group, an alkyl group having 1 to 10 carbon atoms, a cycloalkyl group having 3 to 10 carbon atoms, an aryl group having 6 to 30 carbon atoms, a heteroaryl group having 1 to 30 carbon atoms, an alkylamino group having 1 to 10 carbon atoms, an arylamino group having 6 to 30 carbon atoms, a heteroarylamino group having 1 to 30 carbon atoms, an alkylsilyl group having 1 to 10 carbon atoms, an arylsilyl group having 6 to 30 carbon atoms, an alkoxy group having 1 to 10 carbon atoms, an aryloxy group having 6 to 30 carbon atoms, an alkylthio group having 1 to 10 carbon atoms, and an arylthio group having 6 to 30 carbon atoms;
*a, *bi, and *b₂ are different from each other, and represent bonding positions on a phenylene moiety, and *c represents a bonding position on a phenanthryl moiety;
one of R₇ to R₁₁ represents a single bond that is bonded to *a;
one of R₁₂ to R₁₇ represents a single bond that is bonded to *b₁;
one of R₁₂ to R₁₇ represents a single bond that is bonded to *b₂; and
one of R₁₈ to R₂₇ represents a single bond that is bonded to *c.

2. The organic compound of claim 1, wherein L₁ is selected from the group consisting of a single bond, and a substituted or unsubstituted arylene group having 6 to 15 carbon atoms.

3. The organic compound of claim 1, wherein Ar₁ is selected from the group consisting of a substituted or unsubstituted aryl group having 6 to 30 carbon atoms, and a substituted or unsubstituted heteroaryl group having 1 to 30 carbon atoms, wherein the heteroaryl group includes at least one heteroatom selected from the group consisting of oxygen (O), sulfur (S), and nitrogen (N).

4. The organic compound of claim 1, wherein Ar₂ is a substituted or unsubstituted aryl group having 6 to 15 carbon atoms.

5. An organic light emitting diode comprising:
an anode;
a cathode facing the anode; and
one or more organic layers disposed between the anode and the cathode,
wherein at least one of the organic layers includes the organic compound represented by Chemical Formula 1 of claim 1, and
wherein the organic layer including the organic compound represented by Chemical Formula 1 is a hole transport layer or a hole transport auxiliary layer.
